(19) United States
(12) Patent Application Publication
(10) Pub. No.: US 2007/0299043 A1
(43) Pub. Date: Dec. 27, 2007

(54) Anti-scarring drug combinations and use thereof

(76) Inventors: William L. Hunter, Vancouver (CA); Philip M. Toleikis, Vancouver (CA); David M. Gravett, Vancouver (CA); Daniel S. Grau, Arlington, MA (US); Alexis Borisy, Arlington, MA (US); Curtis T. Keith, Boston, MA (US); Benjamin A. Auspitz, Cambridge, MA (US); M. James Nichols, Boston, MA (US); Edward Roydon Jost-Price, West Roxbury, MA (US); George N. Serbedzija, Sudbury, MA (US)

Correspondence Address: CLARK & ELBING LLP, 101 FEDERAL STREET, BOSTON, MA, 02110, US

(21) Appl. No.: 11/732,808

(22) Filed: Apr. 4, 2007

Related U.S. Application Data
(63) Continuation-in-part of application No. 11/542,185, filed on Oct. 3, 2006.
(60) Provisional application No. 60/723,053, filed on Oct. 3, 2005.

(51) Int. Cl. A61K 31/57; A61P 17/02

(52) U.S. Cl. 514/171

(57) ABSTRACT

The present invention provides devices or implants that comprise anti-scarring drug combinations, methods or making such devices or implants, and methods of inhibiting fibrosis between the devices or implants and tissue surrounding the devices or implants. The present invention also provides compositions that comprise anti-fibrotic drug combinations, and their uses in various medical applications including the prevention of surgical adhesions, treatment of inflammatory arthritis, treatment of scars and keloids, the treatment of vascular disease, and the prevention of cartilage loss.

### CROSS-REFERENCE TO RELATED APPLICATIONS

This application is a continuation-in-part of U.S. application Ser. No. 11/542,185, filed Oct. 3, 2006, which claims benefit of U.S. Provisional Application No. 60/723,053, filed Oct. 3, 2005, each of which is hereby incorporated by reference.

### FIELD OF THE INVENTION

This invention relates generally to devices and compositions that include anti-scarring combinations and to methods of making and using such compositions.

### BACKGROUND OF THE INVENTION

The clinical function of numerous medical implants and devices is dependent upon the device being able to effectively maintain an anatomical, or surgically created, space or passageway. Unfortunately, many devices implanted in the body are subject to a “foreign body” response from the surrounding host tissues. In particular, injury to tubular anatomical structures (such as blood vessels, the gastrointestinal tract, the male and female reproductive tract, the urinary tract, sinuses, spinal nerve root canals, lacrimal ducts, Eustachian tubes, the auditory canal, and the respiratory tract) from surgery and/or injury created by the implantation of medical devices can lead to a well known clinical problem called “stenosis” (or narrowing). Stenosis occurs in response to trauma to the epithelial lining or the entire body tube during the procedure, including virtually any manipulation which attempts to relieve obstruction of the passageway, and is a major factor limiting the effectiveness of invasive treatments for a variety of diseases to be described later.

Stenosis (or “restenosis” if the problem recurs after an initially successful attempt to open a blocked passageway) is a form of response to injury leading to wall thickening, narrowing of the lumen, and loss of function in the tissue supplied by the particular passageway. Physical injury during an interventional procedure results in damage to epithelial lining of the tube and the smooth muscle cells (SMCs) that make up the wall. The damaged cells, particularly SMCs, release cytokines, which recruit inflammatory cells such as macrophages, lymphocytes and neutrophils (i.e., which are some of the known white blood cells) into the area. The white blood cells in turn release a variety of additional cytokines, growth factors, and tissue degrading enzymes that influence the behavior of the constituent cells of the wall (primarily epithelial cells and SMCs). Stimulation of the SMCs induces them to migrate into the inner aspect of the body passageway (often called the “intima”), proliferate and secrete an extracellar matrix—effectively filling all or parts of the lumen with reactive, fibrous scar tissue. Collectively, this creates a thickening of the intimal layer (known in some tissues as “neointimal hyperplasia”) that narrows the lumen of the passageway and can be significant enough to obstruct its lumen.

Polymeric compositions, particularly those that include synthetic polymers or a combination of synthetic and naturally occurring polymers, have been used in a variety of medical applications, such as the prevention of surgical adhesions, tissue engineering, and as bioadhesive materials. U.S. Pat. No. 5,162,430 describes the use of collagen-synthetic polymer conjugates prepared by covalently binding collagen to synthetic hydrophilic polymers such as various derivatives of polyethylene glycol. In a related patent, U.S. Pat. No. 5,328,955, various activated forms of polyethylene glycol and various linkages are described, which can be used to produce collagen-synthetic polymer conjugates having a range of physical and chemical properties. U.S. Pat. No. 5,324,775 also describes synthetic hydrophilic polyethylene glycol conjugates, but the conjugates involve naturally occurring polymers such as polysaccharides. EP 0 732 109 A1 discloses a crosslinked biomaterial composition that is prepared using a hydrophobic crosslinking agent, or a mixture of hydrophilic and hydrophobic crosslinking agents. U.S. Pat. No. 5,614,587 describes bioadhesives that comprise collagen that is crosslinked using a multifunctionally activated synthetic hydrophilic polymer. U.S. application Ser. No. 08/403,360, filed Mar. 14, 1995, discloses a composition useful in the prevention of surgical adhesions comprising a substrate material and an anti-adhesion binding agent, where the substrate material may comprise collagen and the binding agent may comprise at least one tissue-reactive functional group and at least one substrate-reactive functional group. U.S. application Ser. No. 08/476,825, filed Jun. 7, 1995, discloses bioadhesive compositions comprising collagen crosslinked using a multifunctionally activated synthetic hydrophilic polymer, as well as methods of using such compositions to effect adhesion between a first surface and a second surface, wherein at least one of the first and second surfaces may be a native tissue surface. U.S. Pat. No. 5,874,500 describes a crosslinked polymer composition that comprises one component having multiple nucleophilic groups and another component having multiple electrophilic groups. Covalently bonding of the nucleophilic and electrophilic groups forms a three dimensional matrix that has a variety of medical uses including tissue adhesion, surface coatings for synthetic implants, and drug delivery. More recent developments include the addition of a third component having either nucleophilic or electrophilic groups, as is described in U.S. Pat. No. 6,458,889 to Trollsas et al. U.S. Pat. Nos. 5,874,500, 6,051,648 and 6,312,725 disclose the in situ crosslinking or crosslinked polymers, in particular poly(ethylene glycol) based polymers, to produce a crosslinked composition. West and Hubbell, Biomaterials (1995) 16:1153-1156, disclose the prevention of post-operative adhesions using a photopolymerized polyethylene glycol-co-lactic acid diacrylate hydrogel and a physically crosslinked polyethylene glycol-co-polypropylene glycol hydrogel, POLOXAMER 407 (BASF Corporation, Mount Olive, N.J.). Polymerizable cyanoacrylates have also been described for use as tissue adhesives (Ellis, et al., J. Otolaryngol. 19:68-72 (1990)). Two-part synthetic polymer compositions have been described that, when mixed together, form covalent bonds with one another, as well as with exposed tissue surfaces (PCT WO 97/22371, which corresponds to U.S. application Ser. No. 08/769,806 U.S. Pat. No. 5,874,500).

### BRIEF SUMMARY OF THE INVENTION

Briefly stated, in one aspect, the present invention provides compositions for delivery of selected anti-scarring drug combinations (or individual components thereof) via medical implants or implantable medical devices, as well as methods for making and using these implants and devices. Within one aspect of the invention, drug-coated or drug-impregnated implants and medical devices coated or impregnated with anti-scarring drug combinations are provided which reduce fibrosis in the tissue surrounding the device or implant, or inhibit scar development on the device/implant surface, thus enhancing the efficacy the procedure. Within various embodiments, fibrosis is inhibited by local or systemic release of specific anti-fibrosis drug combinations or individual components thereof that become localized to the adjacent tissue.

The repair of tissues following a mechanical or surgical intervention involves two distinct processes: (1) regeneration (the replacement of injured cells by cells of the same type) and (2)fibrosis (the replacement of injured cells by connective tissue). There are four general components to the process of fibrosis (or scarring) including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). Within one embodiment of the invention, an implant or device is adapted to release an agent that inhibits fibrosis or regeneration through one or more of the mechanisms sited herein.

Within yet other aspects of the present invention, methods are provided for manufacturing a medical device or implant, comprising the step of coating (e.g., spraying, dipping, wrapping, or administering drug through) a medical device or implant with anti-fibrosis drug combination (or individual components thereof). Additionally, the implant or medical device can be constructed so that the device itself is comprised of materials that comprise anti-fibrosis drug combinations (or individual components thereof) in or around the implant. A wide variety of medical devices and implants may be utilized within the context of the present invention, depending on the site and nature of treatment desired.

Within related aspects of the present invention, intravascular devices, gastrointestinal stents, tracheal and bronchial stents, genital urinary stents, ear and nose stents, ear ventilation tubes, intraocular implants, devices for treating hypertropic scar or keloid, vascular grafts, hemodialysis access devices, devices comprising a film or a mesh, glaucoma drainage devices, prosthetic heart valves or components thereof, penile implants, endotracheal or tracheostomy tubes, peritoneal dialysis catheters, central nervous system shunts or pressure monitor devices, inferior vena cava filters, gastrointestinal devices, central venous catheters, ventricular assist devices, spinal implants, implants that provide surgical adhesion barriers, and the like are provided comprising an implant or device, wherein the implant or device is in combination with a drug combination (or individual component(s) thereof) which inhibits fibrosis in vivo.

Within various embodiments of the invention, the implant or device is further coated with a composition or compound, which delays the onset of activity of the fibrosis-inhibiting drug combination (or its individual components thereof) for a period of time after implantation. Representative examples of such agents include heparin, PLGA/MePEG, PLA, and polyethylene glycol. Within further embodiments, the fibrosis-inhibiting implant or device is activated before, during, or after deployment (e.g., an inactive agent on the device is first activated to one that reduces or inhibits an in vivo fibrotic reaction).

Within various embodiments of the invention, a device or implant is coated on one aspect, portion or surface with a composition which inhibits fibrosis, as well as being coated with a composition or compound which promotes scarring on another aspect, portion or surface of the device. Representative examples of agents that promote fibrosis and scarring include silk, wool, silica, bleomycin, neomycin, talcum powder, metallic beryllium, and copper as well as analogues and derivatives thereof.

Also provided by the present invention are methods for treating patients undergoing surgical, endoscopic or minimally invasive therapies where a medical device or implant is placed as part of the procedure. As utilized herein, it should be understood that “inhibits fibrosis or stenosis” refers to a statistically significant decrease in the amount of scar tissue in or around the device or an improvement in the luminal area of the device/implant, which may or may not result in a permanent prohibition of any complications or failures of the device/implant.

The pharmaceutical agents and compositions are utilized to create novel implants and medical devices coated with drug combinations or individual components thereof that reduce the foreign body response to implantation and limit the growth of reactive tissue on the surface of, or around in the tissue surrounding the device, such that performance is enhanced. In many instances, the devices are used to maintain body lumens or passageways such as blood vessels, the gastrointestinal tract, the male and female reproductive tract, the urinary tract, bony foramena (e.g., sinuses, spinal nerve root canals, lacrimal ducts, Eustachian tubes, the auditory canal), and the respiratory tract, where obstruction of the device by scar tissue in the post-procedural period leads to the adverse clinical sequela or failure of the intervention. Medical devices and implants coated with selected drug combinations (or individual components thereof) designed to prevent scar tissue overgrowth and preserve patency can offer significant clinical advantages over uncoated devices.

For example, in one aspect the present invention is directed to devices that comprise a medical implant and at least one of (i) an anti-scarring drug combination (or individual component(s) thereof) and (ii) a composition that comprises an anti-scarring drug combination (or individual component(s) thereof). The drug combination is present so as to inhibit scarring that can otherwise occur when the implant is placed within an animal. In another aspect the present invention is directed to methods wherein both an implant and at least one of (i) an anti-scarring drug combination (or individual component(s) thereof) and (ii) a composition that comprises an anti-scarring drug combination (or individual component(s) thereof), are placed into an animal, and the drug combination inhibits scarring that can otherwise occur. These and other aspects of the invention are summarized below.

Thus, in various independent aspects, the present invention provides the following: a device, comprising a medical device and an anti-scarring drug combination or a composition comprising an anti-scarring drug combination, wherein the drug combination inhibits scarring; a device, comprising an intravascular device and an anti-scarring drug combination or a composition comprising an anti-scarring drug combination, wherein the drug combination inhibits scarring; a device, comprising a a gastrointestinal stent and an anti-scarring drug combination or a composition comprising an anti-scarring drug combination, wherein the drug combination inhibits scarring; a device, comprising a tracheal and bronchial stent and an anti-scarring drug combination or a composition comprising an anti-scarring drug combination, wherein the drug combination inhibits scarring; a device, comprising a genital urinary stent and an anti-scarring drug combination or a composition comprising an anti-scarring drug combination, wherein the drug combination inhibits scarring; a device, comprising an ear and nose stent and an anti-scarring drug combination or a composition comprising an anti-scarring drug combination, wherein the drug combination inhibits scarring; a device, comprising an ear ventilation tube and an anti-scarring drug combination or a composition comprising an anti-scarring drug combination, wherein the drug combination inhibits scarring; a device, comprising an intraocular implant and an anti-scarring drug combination or a composition comprising an anti-scarring drug combination, wherein the drug combination inhibits scarring; a device, comprising a medical device for treating hypertropic scar or keloid and an anti-scarring drug combination or a composition comprising an anti-scarring drug combination, wherein the drug combination inhibits scarring; device, comprising a vascular graft and an anti-scarring drug combination or a composition comprising an anti-scarring drug combination, wherein the drug combination inhibits scarring; a device, comprising a hemodialysis access device and an anti-scarring drug combination or a composition comprising an anti-scarring drug combination, wherein the drug combination inhibits scarring; a device, comprising a device comprising a film or a mesh and an anti-scarring drug combination or a composition comprising an anti-scarring drug combination, wherein the drug combination inhibits scarring; a device, comprising glaucoma drainage device and an anti-scarring drug combination or a composition comprising an anti-scarring drug combination, wherein the drug combination inhibits scarring; a device, comprising prosthetic heart valve or component thereof and an anti-scarring drug combination or a composition comprising an anti-scarring drug combination, wherein the drug combination inhibits scarring; a device, comprising a penile implant and an anti-scarring drug combination or a composition comprising an anti-scarring drug combination, wherein the drug combination inhibits scarring; a device, comprising an endotracheal or tracheostomy tube and an anti-scarring drug combination or a composition comprising an anti-scarring drug combination, wherein the drug combination inhibits scarring; a device, comprising a peritoneal dialysis catheter and an anti-scarring drug combination or a composition comprising an anti-scarring drug combination, wherein the drug combination inhibits scarring; a device, comprising a central nervous system shunt or pressure monitor device and an anti-scarring drug combination or a composition comprising an anti-scarring drug combination, wherein the drug combination inhibits scarring; a device, comprising inferior vena cava filter and an anti-scarring drug combination or a composition comprising an anti-scarring drug combination, wherein the drug combination inhibits scarring; a device, comprising a gastrointestinal device and an anti-scarring drug combination or a composition comprising an anti-scarring drug combination, wherein the drug combination inhibits scarring; a device, comprising a central venous catheter and an anti-scarring drug combination or a composition comprising an anti-scarring drug combination, wherein the drug combination inhibits scarring; a device, comprising a ventricular assist device and an anti-scarring drug combination or a composition comprising an anti-scarring drug combination, wherein the drug combination inhibits scarring; a device, comprising a spinal implant and an anti-scarring drug combination or a composition comprising an anti-scarring drug combination, wherein the drug combination inhibits scarring; a device, comprising an implant that provides a surgical adhesion barrier and an anti-scarring drug combination or a composition comprising an anti-scarring drug combination, wherein the drug combination inhibits scarring. These and other devices are described in more detail herein.

In additional aspects, for (1) each of the aforementioned devices combined with (2) each of the anti-fibrotic drug combinations disclosed herein, it is, for each combination, independently disclosed that the anti-fibrotic drug combination may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and drug combination described above, are set forth in greater detail herein.

In addition to devices, the present invention also provides methods. For example, in additional aspects of the present invention, for each of the aforementioned devices, and for each of the aforementioned combinations of the devices with the anti-scarring drug combinations, the present invention provides methods whereby a specified device is implanted into an animal, and a specified drug combination associated with the device inhibits scarring that can otherwise occur is also delivered into the animal. Each of the devices identified herein may be a “specified device”, and each of the anti-scarring drug combinations identified herein may be an “anti-scarring drug combination”, where the present invention provides, in independent embodiments, for each possible combination of the device and the drug combination.

The drug combination may be associated with the device prior to the device being placed within the animal. For example, the drug combination (or composition comprising the drug combination) may be coated onto an implant, and the resulting device then placed within the animal. In addition, or alternatively, the drug combination may be independently placed within the animal in the vicinity of where the device is to be, or is being, placed within the animal. For example, the drug combination may be sprayed or otherwise placed onto the tissue that will be contacting the medical implant or may otherwise undergo scarring. To this end, the present invention provides, in independent aspects: a method for inhibiting scarring comprising placing a medical device and an anti-scarring drug combination or a composition comprising an anti-scarring drug combination into an animal host, wherein the drug combination inhibits scarring; a method for inhibiting scarring comprising placing an intravascular device and an anti-scarring drug combination or a composition comprising an anti-scarring drug combination into an animal host, wherein the drug combination inhibits scarring; a method for inhibiting scarring comprising placing a gastrointestinal stent and an anti-scarring drug combination or a composition comprising an anti-scarring drug combination into an animal host, wherein the drug combination inhibits scarring; a method for inhibiting scarring comprising placing a tracheal and bronchial stent and an anti-scarring drug combination or a composition comprising an anti-scarring drug combination into an animal host, wherein the drug combination inhibits scarring; a method for inhibiting scarring comprising placing a genital urinary stent and an anti-scarring drug combination or a composition comprising an anti-scarring drug combination into an animal host, wherein the drug combination inhibits scarring; a method for inhibiting scarring comprising placing an ear and nose stent and an anti-scarring drug combination or a composition comprising an anti-scarring drug combination into an animal host, wherein the drug combination inhibits scarring; a method for inhibiting scarring comprising placing an ear ventilation tube and an anti-scarring drug combination or a composition comprising an anti-scarring drug combination into an animal host, wherein the drug combination inhibits scarring; a method for inhibiting scarring comprising placing an intraocular implant and an anti-scarring drug combination or a composition comprising an anti-scarring drug combination into an animal host, wherein the drug combination inhibits scarring; a method for inhibiting scarring comprising placing a medical device for treating hypertropic scar or keloid and an anti-scarring drug combination or a composition comprising an anti-scarring drug combination into an animal host, wherein the drug combination inhibits scarring; a method for inhibiting scarring comprising placing a vascular graft and an anti-scarring drug combination or a composition comprising an anti-scarring drug combination into an animal host, wherein the drug combination inhibits scarring; a method for inhibiting scarring comprising placing a hemodialysis access device and an anti-scarring drug combination or a composition comprising an anti-scarring drug combination into an animal host, wherein the drug combination inhibits scarring; a method for inhibiting scarring comprising placing a medical device comprising a film or a mesh and an anti-scarring drug combination or a composition comprising an anti-scarring drug combination into an animal host, wherein the drug combination inhibits scarring; a method for inhibiting scarring comprising placing a glaucoma drainage device and an anti-scarring drug combination or a composition comprising an anti-scarring drug combination into an animal host, wherein the drug combination inhibits scarring; a method for inhibiting scarring comprising placing prosthetic heart valve or component thereof and an anti-scarring drug combination or a composition comprising an anti-scarring drug combination into an animal host, wherein the drug combination inhibits scarring; a method for inhibiting scarring comprising placing a penile implant and an anti-scarring drug combination or a composition comprising an anti-scarring drug combination into an animal host, wherein the drug combination inhibits scarring; a method for inhibiting scarring comprising placing an endotracheal or tracheostomy tube and an anti-scarring drug combination or a composition comprising an anti-scarring drug combination into an animal host, wherein the drug combination inhibits scarring; a method for inhibiting scarring comprising placing a peritoneal dialysis catheter and an anti-scarring drug combination or a composition comprising an anti-scarring drug combination into an animal host, wherein the drug combination inhibits scarring; a method for inhibiting scarring comprising placing a central nervous system shunt or pressure monitor device and an anti-scarring drug combination or a composition comprising an anti-scarring drug combination into an animal host, wherein the drug combination inhibits scarring; a method for inhibiting scarring comprising placing inferior vena cava filter and an anti-scarring drug combination or a composition comprising an anti-scarring drug combination into an animal host, wherein the drug combination inhibits scarring; a method for inhibiting scarring comprising placing a gastrointestinal device and an anti-scarring drug combination or a composition comprising an anti-scarring drug combination into an animal host, wherein the drug combination inhibits scarring; a method for inhibiting scarring comprising placing a central venous catheter and an anti-scarring drug combination or a composition comprising an anti-scarring drug combination into an animal host, wherein the drug combination inhibits scarring; a method for inhibiting scarring comprising placing a ventricular assist device and an anti-scarring drug combination or a composition comprising an anti-scarring drug combination into an animal host, wherein the drug combination inhibits scarring; a method for inhibiting scarring comprising placing a spinal implant and an anti-scarring drug combination or a composition comprising an anti-scarring drug combination into an animal host, wherein the drug combination inhibits scarring; a method for inhibiting scarring comprising placing an implant that provides surgical barrier and an anti-scarring drug combination or a composition comprising an anti-scarring drug combination into an animal host, wherein the drug combination inhibits scarring.

In certain independent aspects, the present invention provides a method for implanting a medical device comprising: (a) infiltrating a tissue of a host where the medical device is to be, or has been, implanted with an anti-scarring drug combination or a composition comprising an anti-scarring drug combination (e.g., a composition comprising an anti-scarring drug combination and a polymer) and (b) implanting the medical device into the host; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is an intravascular device; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a gastrointestinal stent; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a tracheal and bronchial stent; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a genital urinary stent; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is an ear and nose stent; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is an ear ventilation tube; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is an intraocular implant; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a medical device for treating hypertropic scar or keloid; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a vascular graft; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a hemodialysis access device; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a medical device that comprises a film or a mesh; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a glaucoma drainage device; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a prosthetic heart valve or a component thereof, a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a penile implant; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is an endotracheal or tracheostomy tube; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a peritoneal dialysis catheter; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a central nervous system shunt or a pressure monitoring device; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is an inferior vena cava filter; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a gastrointestinal device; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a central venous catheter; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a ventricular assist device; a method for implanting a medical device comprising: (a) infiltrating a tissue of a host where the medical device is to be, or has been, implanted with an anti-scarring drug combination or a composition comprising an anti-scarring drug combination, and (b) implanting the medical device into the host, wherein the medical device is a spinal implant.

In certain independent aspects, the present invention provides a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is an intravascular device; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a gastrointestinal stent; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a tracheal and bronchial stent; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a genital urinary stent; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is an ear and nose stent; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is an ear ventilation tube; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is an intraocular implant; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a medical device for treating hypertropic scar or keloid; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a vascular graft; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a hemodialysis access device; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a medical device that comprises a film or a mesh; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a glaucoma drainage device; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a prosthetic heart valve or a component thereof, a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a penile implant; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is an endotracheal or tracheostomy tube; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a peritoneal dialysis catheter; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a central nervous system shunt or a pressure monitoring device; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is an inferior vena cava filter; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a gastrointestinal device; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a central venous catheter; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a ventricular assist device; a method for implanting a medical device comprising: (a) infiltrating a tissue of a host where the medical device is to be, or has been, implanted with a first compound or a composition comprising a first compound and (b) implanting the medical device that comprises a second compound or a composition comprising a second compound into the host, wherein the first and second compounds form an anti-scarring drug combination, and wherein the medical device is a spinal implant.

In additional aspects, for each of the aforementioned methods used in combination with each of the aforementioned drug combinations, it is, for each combination, independently disclosed that the drug combination may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and drug combination described above, are set forth in greater detail herein.

In independent aspects, the present invention provides a method of making a medical device comprising: combining an intravascular implant and an anti-scarring drug combination or a composition comprising an anti-scarring drug combination, wherein the drug combination inhibits scarring between the device and a host into which the device is implanted; a method of making a medical device comprising: combining a vascular graft or wrap implant and an anti-scarring drug combination or a composition comprising an anti-scarring drug combination, wherein the drug combination inhibits scarring between the device and a host into which the device is implanted; a method of making a medical device comprising: combining an implant for hemodialysis access (i.e., a hemodialysis access device) and an anti-scarring drug combination or a composition comprising an anti-scarring drug combination, wherein the drug combination inhibits scarring between the device and a host into which the device is implanted; a method of making a medical device comprising: combining an implant that provides an anastomotic connection (i.e., an anastomotic connector device) and an anti-scarring drug combination or a composition comprising an anti-scarring drug combination, wherein the drug combination inhibits scarring between the device and a host into which the device is implanted; a method of making a medical device comprising: combining a central venous catheter implant and an anti-scarring drug combination or a composition comprising an anti-scarring drug combination, wherein the drug combination inhibits scarring between the device and a host into which the device is implanted; a method of making a medical device comprising: combining a prosthetic heart valve implant and an anti-scarring drug combination or a composition comprising an anti-scarring drug combination, wherein the drug combination inhibits scarring between the device and a host into which the device is implanted; a method of making a medical device comprising: combining an inferior vena cava filter implant an anti-scarring drug combination or a composition comprising an anti-scarring drug combination, wherein the drug combination inhibits scarring between the device and a host into which the device is implanted; a method of making a medical device comprising: combining a peritoneal dialysis catheter implant and an anti-scarring drug combination or a composition comprising an anti-scarring drug combination, wherein the drug combination inhibits scarring between the device and a host into which the device is implanted; a method of making a medical device comprising: combining an implantable nonvascular stent or tube (i.e., an implant) and an anti-scarring drug combination or a composition comprising an anti-scarring drug combination, wherein the drug combination inhibits scarring between the device and a host into which the device is implanted; a method of making a medical device comprising: combining a central nervous system shunt (i.e., an implant) and an anti-scarring drug combination or a composition comprising an anti-scarring drug combination, wherein the drug combination inhibits scarring between the device and a host into which the device is implanted; a method of making a medical device comprising: combining an intraocular lens (i.e., an implant) and an anti-scarring drug combination or a composition comprising an anti-scarring drug combination, wherein the drug combination inhibits scarring between the device and a host into which the device is implanted; a method of making a medical device comprising: combining a glaucoma drainage device (i.e., an implant) and an anti-scarring drug combination or a composition comprising an anti-scarring drug combination, wherein the drug combination inhibits scarring between the device and a host into which the device is implanted; a method of making a medical device comprising: combining a penile implant and an anti-scarring drug combination or a composition comprising an anti-scarring drug combination, wherein the drug combination inhibits scarring between the device and a host into which the device is implanted; a method of making a medical device comprising: combining an endotracheal tube (i.e., an implant) and an anti-scarring drug combination or a composition comprising an anti-scarring drug combination, wherein the drug combination inhibits scarring between the device and a host into which the device is implanted; a method of making a medical device comprising: combining a tracheostomy tube (i.e., an implant) and an anti-scarring drug combination or a composition comprising an anti-scarring drug combination, wherein the drug combination inhibits scarring between the device and a host into which the device is implanted; a method of making a medical device comprising: combining a gastrointestinal device (i.e., an implant) and an anti-scarring drug combination or a composition comprising an anti-scarring drug combination, wherein the drug combination inhibits scarring between the device and a host into which the device is implanted; a method of making a medical device comprising: combining a spinal implant and an anti-scarring drug combination or a composition comprising an anti-scarring drug combination, wherein the drug combination inhibits scarring between the device and a host into which the device is implanted; a method of making a medical device comprising: combining a pressure monitoring implant and an anti-scarring drug combination or a composition comprising an anti-scarring drug combination, wherein the drug combination inhibits scarring between the device and a host into which the device is implanted; a method of making a medical device comprising: combining a tympanostomy tube implant and an anti-scarring drug combination or a composition comprising an anti-scarring drug combination, wherein the drug combination inhibits scarring between the device and a host into which the device is implanted; a method of making a medical device comprising: combining an implant that provides a surgical adhesion barrier and an anti-scarring drug combination or a composition comprising an anti-scarring drug combination, wherein the drug combination inhibits scarring between the device and a host into which the device is implanted; a method of making a composition comprising surgical adhesion barrier components and an anti-scarring drug combination, wherein the composition inhibits formation of surgical adhesions, and wherein the drug combination inhibits scarring in the vicinity of the composition as it is located within a host that has received the composition; and a method of making a medical device comprising: combining a ventricular assist implant and an anti-scarring drug combination or a composition comprising an anti-scarring drug combination, wherein the drug combination inhibits scarring between the device and a host into which the device is implanted.

In other aspects, the present invention provides compositions that contain both an anti-fibrotic drug combination (or individual component(s) thereof) and either a polymer or a pre-polymer, i.e., a compound that forms a polymer in situ. In one embodiment, these compositions are formed in-situ when precursors thereof are delivered to a site in the body, or a site on an implant. For example, the compositions of the invention include the crosslinked reaction product that forms when two compounds (a multifunctional polynucleophilic compound and a multi-functional polyelectrophilic compound) are delivered to a site in a host (in other words, a patient) in the presence of an anti-fibrotic drug combination (or individual component(s) thereof). However, the compositions of the invention also include a mixture of anti-fibrotic drug combination and a polymer, where the composition can be delivered to a site in a patient's body to achieve beneficial affects, e.g., the beneficial affects described herein.

In one aspect, the present invention provides a composition comprising surgical adhesion barrier components and an anti-scarring drug combination (or individual component(s) thereof), wherein the composition inhibits formation of surgical adhesions, and wherein the drug combination inhibits scarring in the vicinity of the composition as it is located within a host that has received the composition.

In some instances, the polymers themselves are useful in various methods, including the prevention of surgical adhesions.

In another aspect, the present invention provides methods for treating and/or preventing surgical adhesions. For instance, the present invention provides a method for preventing surgical adhesions, comprising delivering a tissue-reactive polymeric composition to a site in need thereof to provide coated tissue, and delivering a fibrosis-inhibiting drug combination to the coated tissue; a method of preventing surgical adhesions, comprising delivering a composition between a dural sleeve and paravertebral musculature in a patient post-laminectomy, where the composition comprises an anti-scarring drug combination and prevents surgical adhesions; a method of preventing surgical adhesions, comprising coating a spinal nerve at a laminectomy site in a patient in need thereof with a composition, where the composition comprises an anti-scarring drug combination and prevents surgical adhesions; a method of preventing surgical adhesions, comprising infiltrating a composition into tissue around a spinal nerve at a laminectomy site in a patient in need thereof, where the composition comprises an anti-scarring drug combination and prevents surgical adhesions; a method of preventing surgical adhesions, comprising delivering a composition to a site of a surgical disc resection in a patient in need thereof, where the composition comprises an anti-scarring drug combination and prevents surgical adhesions; a method of preventing surgical adhesions, comprising delivering a composition to a site of a microdiscectomy in a patient in need thereof, where the composition comprises an anti-scarring drug combination and prevents surgical adhesions; a method of preventing surgical adhesions, comprising delivering a composition to a site of a neurosurgical (brain) procedure in a patient in need thereof, where the composition comprises an anti-scarring drug combination and prevents surgical adhesions; a method of preventing surgical adhesions, comprising infiltrating into a spinal surgical site of a patient in need thereof, a composition that prevents surgical adhesions; a method of preventing surgical adhesions, comprising delivering a composition to epidural tissue in a patient in need thereof, where the composition comprises an anti-scarring drug combination and prevents surgical adhesions; a method of preventing surgical adhesions, comprising delivering a composition to dural tissue in a patient in need thereof, where the composition comprises an anti-scarring drug combination and prevents surgical adhesions; a method of preventing surgical adhesions, comprising delivering a composition to a gynecological site in a patient in need thereof, where the composition comprises an anti-scarring drug combination and prevents surgical adhesions; a method of preventing surgical adhesions, comprising delivering a composition to a tissue surface of the pelvic side wall in a patient in need thereof, where the composition comprises an anti-scarring drug combination and prevents surgical adhesions; a method of preventing surgical adhesions, comprising delivering a composition to a peritoneal cavity in a patient in need thereof, where the composition comprises an anti-scarring drug combination and prevents surgical adhesions; a method of preventing surgical adhesions, comprising delivering a composition to a pelvic cavity in a patient in need thereof, where the composition comprises an anti-scarring drug combination and prevents surgical adhesions; a method of preventing surgical adhesions, comprising delivering a composition to a site of a laparotomy in a patient in need thereof, where the composition comprises an anti-scarring drug combination and prevents surgical adhesions; a method of preventing surgical adhesions, comprising delivering a composition to a site of an endoscopic procedure in a patient in need thereof, where the composition comprises an anti-scarring drug combination and prevents surgical adhesions; a method of preventing surgical adhesions, comprising delivering a composition to a site of a hernia repair in a patient in need thereof, where the composition comprises an anti-scarring drug combination and prevents surgical adhesions; a method of preventing surgical adhesions, comprising delivering a composition to a site of cholecystectomy in a patient in need thereof, where the composition comprises an anti-scarring drug combination and prevents surgical adhesions; a method of preventing surgical adhesions, comprising delivering a composition to a site of a cardiac procedure in a patient in need thereof, where the composition comprises an anti-scarring drug combination and prevents surgical adhesions; a method of preventing surgical adhesions, comprising delivering a composition to a site of cardiac transplant surgery in a patient in need thereof, where the composition comprises an anti-scarring drug combination and prevents surgical adhesions; a method of preventing surgical adhesions, comprising delivering a composition to a site of cardiac vascular repair in a patient in need thereof, where the composition comprises an anti-scarring drug combination and prevents surgical adhesions; a method of preventing surgical adhesions, comprising delivering a composition to a site of a heart valve replacement in a patient in need thereof, where the composition comprises an anti-scarring drug combination and prevents surgical adhesions; a method of preventing pericardial surgical adhesions, comprising delivering a composition to a site of pericardial surgery in a patient in need thereof, where the composition comprises an anti-scarring drug combination and prevents surgical adhesions; a method of preventing surgical adhesions, comprising delivering a composition to a site of an orthopedic surgical procedure in a patient in need thereof, where the composition comprises an anti-scarring drug combination and prevents surgical adhesions; a method of preventing surgical adhesions, comprising delivering a composition to a site of a torn ligament in a patient in need thereof, where the composition comprises an anti-scarring drug combination and prevents surgical adhesions; a method of preventing surgical adhesions, comprising delivering a composition to a site of a joint injury in a patient in need thereof, where the composition comprises an anti-scarring drug combination and prevents surgical adhesions; a method of preventing surgical adhesions, comprising delivering a composition to a site of a tendon injury in a patient in need thereof, where the composition comprises an anti-scarring drug combination and prevents surgical adhesions; a method of preventing surgical adhesions, comprising delivering a composition to a site of a cartilage injury in a patient in need thereof, where the composition comprises an anti-scarring drug combination and prevents surgical adhesions; a method of preventing surgical adhesions, comprising delivering a composition to a site of a muscle injury in a patient in need thereof, where the composition comprises an anti-scarring drug combination and prevents surgical adhesions; a method of preventing surgical adhesions, comprising delivering a composition to a site of a nerve injury in a patient in need thereof, where the composition comprises an anti-scarring drug combination and prevents surgical adhesions; a method of preventing surgical adhesions, comprising delivering a composition to a site of a cosmetic surgical procedure in a patient in need thereof, where the composition comprises an anti-scarring drug combination and prevents surgical adhesions; a method of preventing surgical adhesions, comprising delivering a composition to a site of a reconstructive surgical procedure in a patient in need thereof, where the composition comprises an anti-scarring drug combination and prevents surgical adhesions; a method of preventing surgical adhesions, comprising delivering a composition to a site of a breast implant in a patient in need thereof, where the composition comprises an anti-scarring drug combination and prevents surgical adhesions.

The present invention provides a method for treatment of inflammatory arthritis, comprising delivering to a patient in need thereof a therapeutic composition, the composition comprising a) a polymer and/or a compound that forms a polymer in situ and b) an anti-scarring drug combination; a method for prevention of inflammatory arthritis, comprising delivering to a patient in need thereof a therapeutic composition, the composition comprising a polymer and an anti-scarring drug combination; a method for treatment of osteoarthritis, comprising delivering to a patient in need thereof a therapeutic composition, the composition comprising a polymer and an anti-scarring drug combination; a method for prevention of osteoarthritis, comprising delivering to a patient in need thereof a therapeutic composition, the composition comprising a polymer and an anti-scarring drug combination; a method for treatment of primary osteoarthritis, comprising delivering to a patient in need thereof a therapeutic composition, the composition comprising a polymer and an anti-scarring drug combination; a method for prevention of primary osteoarthritis, comprising delivering to a patient in need thereof a therapeutic composition, the composition comprising a polymer and an anti-scarring drug combination; a method for treatment of secondary osteoarthritis, comprising delivering to a patient in need thereof a therapeutic composition, the composition comprising a polymer and an anti-scarring drug combination; a method for prevention of secondary osteoarthritis, comprising delivering to a patient in need thereof a therapeutic composition, the composition comprising a polymer and an anti-scarring drug combination; a method for treatment of rheumatoid arthritis, comprising delivering to a patient in need thereof a therapeutic composition, the composition comprising a polymer and an anti-scarring drug combination; and a method for prevention of rheumatoid arthritis, comprising delivering to a patient in need thereof a therapeutic composition, the composition comprising a polymer and an anti-scarring drug combination. The method includes delivering to patient in need thereof an anti-fibrotic drug combination, optionally with a polymer.

In another aspect, the present invention provides for the prevention of cartilage loss as can occur, for example after a joint injury. The method includes delivering to the joint of the patient in need therof an anti-fibrotic drug combination, optionally with a polymer. In certain embodiments, the present invention provides a method for reducing cartilage loss following an injury to a joint in a patient in need thereof, comprising delivering to the patient a) an anti-scarring drug combination or b) a composition comprising i) an anti-scarring drug combination and ii) a polymer and/or a compound that forms a polymer in situ; a method for preventing cartilage loss following an injury to a joint in a patient in need thereof, comprising delivering to the patient a) an anti-scarring drug combination or b) a composition comprising i) an anti-scarring drug combination and ii) a polymer and/or a compound that forms a polymer in situ; a method for reducing cartilage loss following a cruciate ligament tear in a patient in need thereof, comprising delivering to the patient a) an anti-scarring drug combination or b) a composition comprising i) an anti-scarring drug combination and ii) a polymer and/or a compound that forms a polymer in situ; a method for preventing cartilage loss following a cruciate ligament tear in a patient in need thereof, comprising delivering to the patient a) an anti-scarring drug combination or b) a composition comprising i) an anti-scarring drug combination and ii) a polymer and/or a compound that forms a polymer in situ; a method for reducing cartilage loss following a meniscal tear in a patient in need thereof, comprising delivering to the patient a) an anti-scarring drug combination or b) a composition comprising i) an anti-scarring drug combination and ii) a polymer and/or a compound that forms a polymer in situ; a method for preventing cartilage loss following a meniscal ligament tear in a patient in need thereof, comprising delivering to the patient a) an anti-scarring drug combination or b) a composition comprising i) an anti-scarring drug combination and ii) a polymer and/or a compound that forms a polymer in situ.

In another aspect, the present invention provides for treating hypertrophic scars and keloids. The method includes delivering to the scar or keloid of the patient in need thereof an anti-fibrotic drug combination, optionally with a polymer. In certain embodiments, the method comprises delivering to the patient a) an anti-scarring drug combination or b) a composition comprising i) an anti-scarring drug combination and ii) a polymer and/or a compound that forms a polymer in situ. In certain other embodiments, the method comprises delivering to the patient a) an anti-scarring drug combination or b) a composition comprising i) an anti-scarring drug combination and ii) a polymer and/or a compound that forms a polymer in situ.

In another aspect, the present invention provides a method for the treatment of vascular disease, e.g., stenosis, restenosis or atherosclerosis. In certain embodiments, the method includes the perivascular delivery of an anti-fibrotic drug combination. In certain embodiments, the present invention provides a method for treating vascular disease in a patient in need thereof, comprising delivering to the patient a) an anti-scarring drug combination or b) a composition comprising i) an anti-scarring drug combination and ii) a polymer and/or a compound that forms a polymer in situ; a method for treating stenosis in a patient in need thereof, comprising delivering to the patient a) an anti-scarring drug combination or b) a composition comprising i) an anti-scarring drug combination and ii) a polymer and/or a compound that forms a polymer in situ; a method for treating restenosis in a patient in need thereof, comprising delivering to the patient a) an anti-scarring drug combination or b) a composition comprising i) an anti-scarring drug combination and ii) a polymer and/or a compound that forms a polymer in situ; a method for treating atherosclerosis in a patient in need thereof, comprising delivering to the patient a) an anti-scarring drug combination or b) a composition comprising i) an anti-scarring drug combination and ii) a polymer and/or a compound that forms a polymer in situ.

In each of the aforementioned devices, compositions, methods of making the aforementioned devices or compositions, and methods of using the aforementioned devices or compostions, the present invention provides that the anti-fibrotic drug combination may be one or more of the following: 1) an anti-fibrotic drug combination that inhibits cell regeneration, 2) an anti-fibrotic drug combination that inhibits angiogenesis, 3) an anti-fibrotic drug combination that inhibits migration of fibroblasts and/or smooth muscle cells, 4) an anti-fibrotic drug combination that inhibits proliferation of fibroblasts, smooth muscle cells, endothelial cells, macrophages, and/or synovial cells, 5) an anti-fibrotic drug combination that inhibits deposition of extracellular matrix, 6) an anti-fibrotic drug combination inhibits tissue remodeling, 7) an anti-fibrotic drug combination that inhibits the production or effects of cytokine(s) or chemokine(s).

Exemplary anti-fibrotic drug combinations include, but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Additional exemplary anti-fibrotic drug combinations include, but are not limited to, (1) a triazole (e.g., fluconazole or itraconazole) and (2) a aminopyridine (e.g., phenazopyridine (PZP), phenothiazine, dacarbazine, phenelzine); (1) an antiprotozoal (e.g., pentamidine) and (2) a diaminopyridine (e.g., phenazopyridine) or a quaternary ammonium compound (e.g., pentolinium); (1) an aromatic diamidine and (2) an antiestrogen, an anti-fungal imidazole, disulfiram, or ribavirin; (1) an aminopyridine and (2) phenothiazine, dacarbazine, or phenelzine; (1) a quaternary ammonium compound and (2) an anti-fungal imidazole, haloprogin, MnSO₄, or ZnCl₂; (1) an antiestrogen and (2) phenothiazine, cupric chloride, dacarbazine, methoxsalen, or phenelzine; (1) an antifungal imidazone and (2) disulfiram or ribavirin; (1) an estrogenic compound and dacarbazine; (1) amphotericin B and (2) dithiocarbamoyl disulfide (e.g., disulfiram); (1) terbinafine and (2) a manganese compound; (1) a tricyclic antidepreseant (TCA) (e.g., amoxapine) and (2) a corticosteroid (e.g., prednisolone, glucocorticoid, mineralocorticoid); (1) a tetra-substituted pyrimidopyrimidine (e.g., dipyridamole) and (2) a corticosteroid (e.g., fludrocortisone or prednisolone); (1) a prostaglandin (e.g., alprostadil) and (2) a retinoid (e.g., tretinoin (vitamin A)); (1) an azole (e.g., imidazone or triazole) and (2) a steroid (e.g., corticosteroids including glucocorticoid or mineralocorticoid); (1) a steroid and (2) a prostaglandin, beta-adrenergic receptor ligand, anti-mitotic agent, or microtubule inhibitor; (1) a serotonin norepinephrine reuptake inhibitor (SNRI) or naradrenaline reuptake inhibitor (NARI) and (2) a corticosteroid; (1) a non-steroidal immunophilin-dependent immunosuppressant (NSIDI) (e.g., calcineurin inhibitor including cyclosporin, tacrolimus, ascomycin, pimecrolimus, ISAtx 247) and (2) a non-steroidal immunophilin-dependent immunosuppressant enhancer (NSIDIE) (e.g., selective serotonin reuptake inhibitors, tricyclic antidepressants, phenoxy phenols, anti-histamine, phenothiazines, or mu opioid receptor agonists); (1) an antihistamines and (2) an additional agent selected from corticosteroids, tricyclic or tetracyclic antidepressants, selective serotonin reuptake inhibitors, and steroid receptor modulators; (1) a tricyclic compound and (2) a corticosteroid; (1) an antipsychotic drug (e.g., chlorpromazine) and (2) an antiprotozoal drug (e.g., pentamidine); (1) an antihelmintic drug (e.g., benzimidazole) and (2) an antiprotozoal drug (e.g., pentamidine); (1) ciclopirox and (2) an antiproliferative agent; (1) a salicylanilide (e.g., niclosamide) and (2) an antiproliferative agents; (1) pentamidine or its analogue and (2) chlorpromazine or its analogue; (1) an antihelmintic drug (e.g., alberdazole, mebendazole, oxibendazole) and (2) an antiprotozoal drug (e.g., pentamidine); (1) a dibucaine or amide local anaesthetic related to bupivacaine and (2) a vinca alkaloid; (1) pentamidine, analogue or metabolite thereof and (2) an antiproliferative agent; (1) a triazole (e.g., itraconazole) and (2) an antiarrhythmic agents (e.g., amiodarone, nicardipine or bepridil); (1) an azole and (2) an HMG-CoA reductase inhibitor; a phenothiazine conjugate (e.g., a conjugate of phenothiazine and an antiproliferative agent; (1) phenothiazine and (2) an antiproliferative agent; (1) a kinesin inhibitor (e.g., phenothiazine, analog or metabolite) and (2) an antiproliferative agent (e.g., Group A and Group B antiproliferative agents); and (1) an agent that reduces the biological activity of a mitotic kinesin (e.g., chlorpromazine) and (2) an agent that reduces the biological activity of protein tyrosine phosphatase.

Additional exemplary drug combinations may comprise: (1) an anti-inflammatory agent (e.g., steroids) and (2) an agent selected from an anti-depressant, an SSRI, a cardiovascular agent (e.g., an antiplatelet agent), an anti-fungal agent, and prostaglandin, (1) a cardiovascular drug and (2) an antidepressant; (1) a cardiovascular drug and (2) a phosphodiesterase IV inhibitor; (1) an antidepressant and (2) an antihistamine; (1) an anti-fungal agent and (2) an HMG-CoA reductase inhibitor; (1) an antifungal agent and (2) a metal ion (e.g., a manganese ion); and (1) a sedative and (2) an antibiotic.

These and other agents are described in more detail herein.

These and other aspects of the present invention will become evident upon reference to the following detailed description and attached drawings. In addition, various references are set forth herein which describe in more detail certain procedures and/or compositions, and are therefore incorporated by reference in the entirety.

### BRIEF DESCRIPTION OF THE DRAWINGS

FIG. 1 schematically depicts the transcriptional regulation of matrix metalloproteinases.

FIG. 2 is a blot which demonstrates that IL-1 stimulates AP-1 transcriptional activity.

FIG. 3 is a graph which shows that IL-1 induced binding activity decreased in lysates from chondrocytes which were pretreated with paclitaxel.

FIG. 4 is a blot which shows that IL-1 induction increases collagenase and stromelysin in RNA levels in chondrocytes, and that this induction can be inhibited by pretreatment with paclitaxel.

FIGS. 5A-H are blots that show the effect of various anti-microtubule agents in inhibiting collagenase expression.

FIG. 6 is a graph showing the results of a screening assay for assessing the effect of paclitaxel on smooth muscle cell migration.

FIG. 7 is a graph showing the average rank of joint scores of Hartley guinea pig knees with ACL damage treated with paclitaxel. A reduction in score indicates an improvement in cartilage score. The dose response trend is statistically significant (p<0.02).

FIGS. 8A-C are examples of cross sections of Hartley guinea pig knees of control and paclitaxel treated animals. FIG. 8A. Control speciment showing erosion of cartilage to the bone. FIG. 8B. Paclitaxel dose 1 (low dose) showing fraying of cartilage. FIG. 8C. Paclitaxel dose 2 (medium dose) showing minor defects to cartilage.

FIGS. 9A-F are safranin-O stained histological slides of representative synovial tissues from naive (healthy) knees (FIGS. 9A and 9D) and knees with arthritis induced by administration of albumin in Freund's complete adjuvant (FIGS. 9B and 9C) or carrageenan (FIGS. 9E and 9F). Arthritic knees received either control (FIGS. 9B and 9E) or 20% paclitaxel-loaded microspheres (FIGS. 9C and 9F). The data illustrate decreased proteoglycan red staining in arthritic knees treated with control microspheres and the proteoglycan protection properties of the paclitaxel-loaded formulation.

### DETAILED DESCRIPTION OF THE INVENTION

Definitions

Prior to setting forth the invention, it may be helpful to an understanding thereof to first set forth definitions of certain terms that are used herein.

“Fibrosis,” or “scarring,” or “fibrotic response” refers to the formation of fibrous (scar) tissue in response to injury or medical intervention.

“Inhibit fibrosis”, “reduce fibrosis”, “inhibits scarring” and the like are used synonymously to refer to the action of agents or compositions which result in a statistically significant decrease in the formation of fibrous tissue that can be expected to occur in the absence of the agent or composition.

Therapeutic agents which inhibit fibrosis or scarring are referred to herein as “fibrosis-inhibiting agents”, “fibrosis-inhibitors”, “anti-scarring agents”, and the like, where these agents inhibit fibrosis through one or more mechanisms including: inhibiting inflammation or the acute inflammatory response, inhibiting migration or proliferation of connective tissue cells (such as fibroblasts, smooth muscle cells, vascular smooth muscle cells), inhibiting angiogenesis, reducing extracellular matrix (ECM) production or promoting ECM breakdown, and/or inhibiting tissue remodeling.

“Anti-scarring drug combination” (used interchangeably with “fibrosis-inhibiting drug combination,” “anti-fibrosis drug combination,” “anti-fibrotic drug combination,” or the like) refers to a combination or conjugate of two or more therapeutic agents (also referred to as “individual components”) wherein the combination or conjugate inhibits fibrosis or scarring. Such therapeutic agents (i.e., individual components) either have anti-fibrosis activities themselves, or enhance anti-fibrosis activities of other agents in the drug combinations. In certain embodiments, each of the therapeutic agents of an anti-scarring drug combination has anti-fibrosis activities. In certain embodiments, one or more therapeutic agent(s) of an anti-scarring drug combination enhance the anti-fibrosis activities of the other therapeutic agent(s) of the combination. In certain embodiments, one or more therapeutic agent(s) of an anti-scarring drug combination, when combined with the other therapeutic agent(s), produce synergistic anti-fibrosis effects.

When scarring occurs in a confined space (e.g., within a lumen) following surgery or instrumentation (including implantation of a medical device or implant), such that a body passageway (e.g., a blood vessel, the gastrointestinal tract, the respiratory tract, the urinary tract, the female or male reproductive tract, the eustacian tube, etc.) is partially or completely obstructed by scar tissue, this is referred to as “stenosis” (narrowing). When scarring subsequently occurs to re-occlude a body passageway after it was initially successfully opened by a surgical intervention (such as placement of a medical device or implant), this is referred to as “restenosis.”

The compositions of the present invention may further comprise other pharmaceutical active agents. Such “other pharmaceutically active agents” (also referred to as “other biologically active agents,” or “secondary agents”) refers to agents that do not have anti-scarring activities or enhance the anti-scarring activities of another agent, but are beneficial to be used in conjunction with an anti-scarring drug combination under certain circumstances. Those agents include, by way of example and not limitation, anti-thrombotic agents, anti-proliferative agents, anti-inflammatory agents, analgesics, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

“Host”, “person”, “subject”, “patient” and the like are used synonymously to refer to the living being into which a device or implant of the present invention is implanted.

“Implanted” refers to having completely or partially placed a device or implant within a host. A device is partially implanted when some of the device reaches, or extends to the outside of, a host.

“Anti-infective agent” refers to an agent or composition which prevents microrganisms from growing and/or slows the growth rate of microorganisms and/or is directly toxic to microorganisms at or near the site of the agent. These processes would be expected to occur at a statistically significant level at or near the site of the agent or composition relative to the effect in the absence of the agent or composition.

“Inhibit infection” refers to the ability of an agent or composition to prevent microorganisms from accumulating and/or proliferating near or at the site of the agent. These processes would be expected to occur at a statistically significant level at or near the site of the agent or composition relative to the effect in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent which prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent which stimulates a biological process or rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or other molecule which impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995).

“Medical device”, “implant”, “device”, “medical implant”, “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing, replacing, or augmenting etc. damaged or diseased organs and tissues. While normally composed of biologically compatible synthetic materials (e.g., medical-grade stainless steel, titanium and other metals; polymers such as polyurethane, silicon, PLA, PLGA and other materials) that are exogenous, some medical devices and implants include materials derived from animals (e.g., “xenografts” such as whole animal organs; animal tissues such as heart valves; naturally occurring or chemically-modified molecules such as collagen, hyaluronic acid, proteins, carbohydrates and others), human donors (e.g., “allografts” such as whole organs; tissues such as bone grafts, skin grafts and others), or from the patients themselves (e.g., “autografts” such as saphenous vein grafts, skin grafts, tendon/ligament/muscle transplants). Representative examples of medical devices that are of particular utility in the present invention include, but are not restricted to, vascular stents, gastrointestinal stents, tracheal/bronchial stents, genital-urinary stents, ENT stents, intra-articular implants, intraocular lenses, implants for hypertrophic scars and keloids, vascular grafts, anastomotic connector devices, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVC's), ventricular assist devices (e.g., LVAD), spinal prostheses, urinary (Foley) catheters, prosthetic bladder sphincters, orthopedic implants, and gastrointestinal drainage tubes.

“Chondroprotection” refers to the prevention of cartilage loss. Cartilage is formed from chondrocytes, and chondroprotection is the protection of the chrondrocytes so that they do not die.

“Release of an agent from an implant/device” refers to any statistically significant presence of the agent, or a subcomponent thereof, which has dissociated from the implant/device.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

“Hyaluronic acid” or “HA” as used herein refers to all forms of hyaluronic acid that are described or referenced herein, including those that have been processed or chemically or physically modified, as well as hyaluronic acid that has been crosslinked (for example, covalently, ionically, thermally or physically). HA is a glycosaminoglycan composed of a linear chain of about 2500 repeating disaccharide units. Each disaccharide unit is composed of an N-acetylglucosamine residue linked to a glucuronic acid. Hyaluronic acid is a natural substance that is found in the extracellular matrix of many tissues including synovial joint fluid, the vitreous humor of the eye, cartilage, blood vessels, skin and the umbilical cord. Commercial forms of hyaluronic acid having a molecular weight of approximately 1.2 to 1.5 million Daltons (Da) are extracted from rooster combs and other animal sources. Other sources of HA include HA that is isolated from cell culture/fermentation processes. Lower molecular weight HA formulations are also available from a variety of commercial sources. The molecule can be of variable lengths (i.e., different numbers of repeating disaccharide units and different chain branching patterns) and can be modified at several sites (through the addition or subtraction of different functional groups) without deviating from the scope of the present invention.

The term “inter-react” refers to the formulation of covalent bonds, noncovalent bonds, or both. The term thus includes crosslinking, which involves both intermolecular crosslinks and optionally intramolecular crosslinks as well, arising from the formation of covalent bonds. Covalent bonding between two reactive groups may be direct, in which case an atom in reactive group is directly bound to an atom in the other reactive group, or it may be indirect, through a linking group. Noncovalent bonds include ionic (electrostatic) bonds, hydrogen bonds, or the association of hydrophobic molecular segments, which may be the same or different. A crosslinked matrix may, in addition to covalent bonds, also include such intermolecular and/or intramolecular noncovalent bonds.

When referring to polymers, the terms “hydrophilic” and “hydrophobic” are generally defined in terms of an HLB value, i. e., a hydrophilic lipophilic balance. A high HLB value indicates a hydrophilic compound, while a low HLB value characterizes a hydrophobic compound. HLB values are well known in the art, and generally range from 1 to 18. Preferred multifunctional compound cores are hydrophilic, although as long as the multifunctional compound as a whole contains at least one hydrophilic component, crosslinkable hydrophobic components may also be present.

The term “synthetic” is used to refer to polymers, compounds and other such materials that are “chemically synthesized.” For example, a synthetic material in the present compositions may have a molecular structure that is identical to a naturally occurring material, but the material per se, as incorporated in the compositions of the invention, has been chemically synthesized in the laboratory or industrially. “Synthetic” materials also include semi-synthetic materials, i.e., naturally occurring materials, obtained from a natural source, that have been chemically modified in some way. Generally, however, the synthetic materials herein are purely synthetic, i.e., they are neither semi-synthetic nor have a structure that is identical to that of a naturally occurring material.

The term “effective amount” refers to the amount of composition required in order to obtain the effect desired. For example, an “effective amount for inhibiting fibosis” of a composition refers to the amount needed to inhibit fibrosis to a detectable degree. The actual amount that is determined to be an effective amount will vary depending on factors such as the size, condition, sex and age of the patient and can be more readily determined by the caregiver.

The term “in situ” as used herein means at the site of administration. Thus, compositions of the invention can be injected or otherwise applied to a specific site within a patient's body, e.g., a site in need of augmentation, and allowed to crosslink at the site of injection. Suitable sites will generally be intradermal or subcutaneous regions for augmenting dermal support, at a bone fracture site for bone repair, within sphincter tissue for sphincter augmentation (e.g., for restoration of continence), within a wound or suture, to promote tissue regrowth; and within or adjacent to vessel anastomoses, to promote vessel regrowth.

The term “aqueous medium” includes solutions, suspensions, dispersions, colloids, and the like containing water. The term “aqueous environment” means an environment containing an aqueous medium. Similarly, the term “dry environment” means an environment that does not contain an aqueous medium.

With regard to nomenclature pertinent to molecular structures, the following definitions apply:

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The C₁₋₇ alkyl group may be substituted or unsubstituted. C₁₋₇ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

The term “lower alkyl” intends an alkyl group of one to six carbon atoms, preferably one to four carbon atoms.

“Substituted alkyl” refers to alkyl substituted with one or more substituent groups.

“Alkylene,” “lower alkylene” and “substituted alkylene” refer to divalent alkyl, lower alkyl, and substituted alkyl groups, respectively.

The term “aryl” as used herein, and unless otherwise specified, refers to an aromatic substituent containing a single aromatic ring (monocyclic) or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like.

“Substituted aryl” refers to an aryl moiety substituted with one or more substituent groups.

The terms “heteroatom-containing aryl” and “heteroaryl” refer to aryl in which at least one carbon atom is replaced with a heteroatom. The terms “arylene” and “substituted arylene” refer to divalent aryl and substituted aryl groups as just defined.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon.

“Hydrocarbyl” refers to univalent hydrocarbyl radicals containing 1 to about carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. The term “hydrocarbylene” intends a divalent hydrocarbyl moiety containing 1 to about 30 carbon atoms, preferably 1 to about 24 carbon atoms, most preferably 1 to about 12 carbon atoms, including branched or unbranched, saturated or unsaturated species, or the like. The term “lower hydrocarbylene” intends a hydrocarbylene group of one to six carbon atoms, preferably one to four carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and “heterohydrocarbylene” refer to hydrocarbylene in which at least one carbon atom is replaced with a heteroatom. If not otherwise indicated, “hydrocarbyl” indicates both unsubstituted and substituted hydrocarbyls, “heteroatom-containing hydrocarbyl” indicates both unsubstituted and substituted heteroatom-containing hydrocarbyls and so forth.

By “C₂₋₇ alkenyl” is meant a branched or unbranched hydrocarbon group containing one or more double bonds and having from 2 to 7 carbon atoms. A C₂₋₇ alkenyl may optionally include monocyclic or polycyclic rings, in which each ring desirably has from three to six members. The C₂₋₇ alkenyl group may be substituted or unsubstituted. C₂₋₇ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “C₂₋₇ alkynyl” is meant a branched or unbranched hydrocarbon group containing one or more triple bonds and having from 2 to 7 carbon atoms. A C₂₋₇ alkynyl may optionally include monocyclic, bicyclic, or tricyclic rings, in which each ring desirably has five or six members. The C₂₋₇ alkynyl group may be substituted or unsubstituted. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

By “C₂₋₆ heterocyclyl” is meant a stable 5- to 7-membered monocyclic or 7-to 14-membered bicyclic heterocyclic ring which is saturated partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. The nitrogen and sulfur heteroatoms may optionally be oxidized. The heterocyclic ring may be covalently attached via any heteroatom or carbon atom that results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 1,4,5,6-tetrahydro pyridinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, 1,4,5,6-tetrahydro pyridinyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “amido” is meant a chemical substituent of the formula —NRR′, wherein the nitrogen atom is part of an amide bond (e.g., —C(O)—NRR′) and wherein R and R′ are each, independently, selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl, or —NRR′ forms a C₂₋₆ heterocyclyl ring, as defined above, but containing at least one nitrogen atom, such as piperidino, morpholino, and azabicyclo, among others.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R″′)⁺, wherein R, R′, R″, and R″′ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

By “carbo(C₆-C₆ alkoxy)” is meant an ester fragment of the structure CO₂R, wherein R is an alkyl group.

By “carbo(C₁-C₁₈ aryl-C₁-C₆ alkoxy)” is meant an ester fragment of the structure CO₂R, wherein R is an alkaryl group.

By “aryl” is meant a C₆-C₁₈ carbocyclic aromatic ring or ring system. Examples of aryl groups include phenyl, naphthyl, biphenyl, fluorenyl, and indenyl groups. The term “heteroaryl” means a C₁C₉ aromatic ring or ring systems that contains at least one ring heteroatom (e.g., O, S, N). Heteroaryl groups include furyl, thienyl, pyridyl, quinolinyl, tetrazolyl, and imidazolyl groups.

By “halide” or “halogen” is meant bromine, chlorine, iodine, or fluorine.

By “heterocycle” is meant a C₁-C₉ non-aromatic ring or ring system that contains at least one ring heteroatom (e.g., O, S, N). Heterocycles include, for example, pyrrolidinyl, tetrahydrofuranyl, morpholinyl, thiazolidinyl, and imidazolidinyl groups.

Aryl, hetero, and heterocycle groups may be unsubstituted or substituted by one or more substituents selected from the group consisting of C₁-₆ alkyl, hydroxy, halo, nitro, C₁₋₆ alkoxy, C₁₋₆ alkylthio, trihalomethyl, C₁₋₆ acyl, carbonyl, heteroarylcarbonyl, nitrile, C₁₋₆ alkoxycarbonyl, oxo, alkyl (wherein the alkyl group has from 1 to 6 carbon atoms) and heteroarylalkyl (wherein the alkyl group has from 1 to 6 carbon atoms).

By “aromatic residue” is meant an aromatic group having a ring system with conjugated π electrons (e.g., phenyl, or imidazole ). The ring of the aryl group is preferably 5 to 10 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “non-vicinal O, S, or N” is meant an oxygen, sulfur, or substituted or unsubstituted nitrogen heteroatom substituent in a linkage, wherein the heteroatom substituent does not form a bond to a saturated carbon that is bonded to another heteroatom.

The term “substituted” as used herein means any of the above groups (e.g. alkyl, alkoxy, acyl, aryl, heteroaryl and heterocycle) wherein at least one hydrogen atom is replaced with a substituent. In the case of an oxo substituent (“═O”) two hydrogen atoms are replaced. Substituents include halogen, hydroxy, oxo, alkyl, aryl, alkoxy, aryloxy, acyl, mercapto, cyano, alkylthio, arylthio, heteroarylthio, heteroaryl, heterocycle, —NRₐR_{b}, —NRₐC(═O)R_{b}, —NR_{c}C(═O)NRₐR_{b}, —NRₐC(═O)OR_{b}, —NRₐSO₂R_{b}, —C(═O)NRₐR_{b}, —OC(═O)Rₐ, —OC(═O)ORₐ, —OC(═O)NRₐR_{b}, —NRₐSO₂R_{b} or a radical of the formula —Y-Z-Rₐ where Y is alkanediyl, substituted alkanediyl or a direct bond, alkanediyl refers to a divalent alkyl with two hydrogen atoms taken from the same or different carbon atoms, Z is —O—, —S—, —S(═O)—, —S(═O)₂₋, —N(R_{b})—, —C(═O)—, —C(═O)O—, —OC(═O)—, —N(R_{b})C(═O)—, —C(═O)N(R_{b})— or a direct bond, wherein Rₐ, R_{b} and R_{c} are the same or different and independently hydrogen, amino, alkyl, substituted alkyl (including halogenated alkyl), aryl, substituted aryl, heteroaryl, substituted heteroaryl, heterocycle or substituted heterocycle or wherein Rₐ and R_{b} taken together with the nitrogen atom to which they are attached form a heterocycle or substituted heterocycle.

Unless otherwise indicated, it is to be understood that specified molecular segments can be substituted with one or more substituents that do not compromise a compound's utility. For example, “succinimidyl” is intended to include unsubstituted succinimidyl as well as sulfosuccinimidyl and other succinimidyl groups substituted on a ring carbon atom, e.g., with alkoxy substituents, polyether substituents, or the like.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. As used herein, the term “about” refers to +15% of any indicated structure, value, or range.

“A” and “an” refer to one or more of the indicated items. For example, “a” polymer refers to both one polymer or a mixture comprising two or more polymers; “a multifunctional compound” refers not only to a single multifunctional compound but also to a combination of two or more of the same or different multifunctional compounds; “a reactive group” refers to a combination of reactive groups as well as to a single reactive group, and the like.

As discussed above, the present invention provides polymeric compositions which greatly increase the ability to inhibit the formation of reactive scar tissue on, or around, the surface of a device or implant or at a treatment site. Numerous polymeric compositions and therapeutic agents are described herein.

The present invention provides for the combination of compositions (e.g., polymers) which include anti-scarring drug combinations, described below. Also described in more detail below are methods for making and methods for utilizing such compositions.

Anti-Scarring Drug Combinations

In one aspect, the present application provides various anti-scarring drug combinations. In certain embodiments, one therapeutic agent of an anti-scarring drug combination enhances the anti-scarring activities of the other therapeutic agent(s) in the combination. In certain embodiments, each of the therapeutic agents of an anti-scarring drug combination has anti-scarring activities. In certain embodiments, one therapeutic agent in an anti-scarring drug combination produces a synergistic anti-scarring effect with the other therapeutic agent(s) in an anti-scarring drug combination.

In certain embodiments, individual therapeutic agents in the anti-scarring drug combinations of the present invention may be an antidepressant, steoid, anti-platelet agent, antifungal agent, prostaglandin, phosphodiesterase IV inhibitor, antihistamine agent, HMG—CoA reductase inhibitor, metal ion, ismotic laxative, selective serotonin reuptake inhibitor (SSRI), vasodilator, antipsychotic, ophthalmic, anti-mycotic agent, mucosal or dental anesthetic, dopaminergic agent, anti-protozoal, antiestrogen, noradrenaline reuptake inhibitor, non-steroidal immunophilin-dependent immunosuppressant (NSIDI), non-steroidal immunophilin-dependent immunosuppressant enhancer (NSIDIE), antihelmintic drug, antiproliferative agent, antiarrhythmic agent, phenothiazine conjugate, kinesin inhibitor, an agent that reduces the biological activity fo a mitotic kinesin, or an agent that reduces the biological activity of protein tyrosine phosphatase.

In certain embodiments, the anti-scarring drug combinations of the present invention comprise two therapeutic agents that either themselves having anti-scarring activities or enhance the anti-scarring activities of other agents. In certain embodiments, the anti-scarring drug combinations of the present invention comprise three, four, five or more such therapeutic agents.

In one aspect, the present invention discloses drug combinations that inhibit one or more aspects of the production of excessive fibrous (scar) tissue. Suitable fibrosis-inhibiting or stenosis-inhibiting drug combinations (or individual components thereof) may be readily determined based upon the in vitro and in vivo (animal) models such as those provided in Examples 16-29, 38, 39, and 43. Agents which inhibit fibrosis may be identified through in vivo models including inhibition of intimal hyperplasia development in the rat balloon carotid artery model (Examples 21 and 29). The assays set forth in Examples 20 and 28 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰M. The assay set forth in Example 24 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. Assays set forth herein may be used to determine whether an agent is able to inhibit inflammatory processes, including nitric oxide production in macrophages (Example 16), and/or TNF-alpha production by macrophages (Example 17), and/or IL-1 beta production by macrophages (Example 25), and/or IL-8 production by macrophages (Example 26), and/or inhibition of MCP-1 by macrophages (Example 27). In one aspect of the invention, the agent has an IC₅₀ for inhibition of any one of these inflammatory processes within a range of about 10⁻⁶ to about 10⁻¹⁰M. The assay set forth in Example 22 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 23 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. Agents which reduce the formation of surgical adhesions may be identified through in vivo models including the rabbit surgical adhesions model (Examples 19, 38, 39, and 43) and the rat caecal sidewall model (Example 18). These pharmacologically active agents (described below) can then be delivered at appropriate dosages into to the tissue either alone, or via carriers (described herein), to treat the clinical problems described herein.

Compounds useful in the invention include those described herein in any of their pharmaceutically acceptable forms, including isomers such as diastereomers and enantiomers, salts, solvates, and polymorphs, thereof, as well as racemic mixtures of the compounds described herein. Structural or functional analogs or metabolites of these compounds may also be used.

In certain embodiments, one or more of the components of the drug combinations of the present invention are approved by a national pharmaceutical regulatory agency, such as the United States Food and Drug Administration (USFDA) for administration to a human.

Certain exemplary drug combinations described below are also described in the following publications of U.S. and PCT patent applications (which are incorporated in their entireties by reference): WO 02/58697, WO 03/06026, WO 03/30823, WO 03/57162, WO 03/66049, WO 03/03580, WO 03/92617, WO 04/002430, WO 04/007676, WO 04/006906, WO 02/006842, WO 04/006849, WO 04/030618, US 2004/157837, WO 04/073631, WO 04/073614, WO 05/011572, WO 04/105696, WO 05/000208, WO 05/027839, WO 05/020913, WO 05/027842, WO 05/048927, WO 05/053613, and WO 05/046607. Exemplary classes of drug combinations are provided below. For each class of drug combinations, the present invention includes each combination of individual components described herein that has anti-scarring activity.

Exemplary drug combinations are described in more detail below. In the following description of exemplary drug combinations, unless otherwise noted, the numbering of chemical formulas is limited to the section related to the particular drug combination where the formulas are present. Put differently, a same numbered formula may represent different chemical structures in sections describing different drug combinations.

Combination Comprising Amoxapine and Prednisolone

In certain embodiments, the drug combination according to the present invention comprises amoxapine (an antidepressant) and prednisolone (a steroid).

Prednisolone has the following structure:

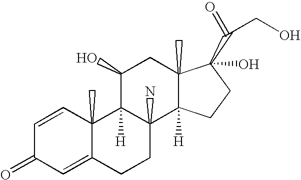

Amoxapine has the following structure:

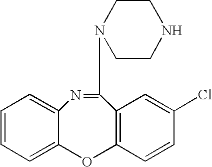

This drug combination is in clinical phase IIa trials in the United States.

Preclinical data suggest that when administered together, amoxapine synergistically increases the immuno-modulatory activity of the reduced-dose steroid without a comparable increase in its adverse side effects, indicating that this drug combination may have a superior risk-to-benefit ratio compared to traditional steroids.

In vitro, this drug combination synergistically inhibits TNF-α release from stimulated primary human lymphocytes as measured by Loewe and other standard synergy models. It also synergistically inhibits IFN-γ and IL-2 in vitro. Although not wishing to be bound by any particular theories, it is believed that the increased activity of the reduced-dose steroid in this drug combination occurs in part through action involving T-cells.

The mechanism studies of this drug combination show amoxapine does not promote glucocorticoid receptor trafficking and does not potentiate prednisolone's ability to transactivate a transfected GRE reporter plasmid in T cells. Amoxapine is observed to block NFAT activation, translocation and transactivation, effects not observed with prednisolone. Amoxapine partially inhibits NFkB and AP1 activation (at low potency), an effect also observed with prednisolone. Inhibition of p38 and JNK activation by amoxapine is observed, whereas ERK is unaffected. These data support a mechanistic model in which amoxapine plays a synergistic immuno-modulatory role in this drug combination by selectively enhancing a subset of prednisolone's actions on pathways of T cell activation.

In both acute and chronic in vivo models of inflammation, amoxapine alone and reduced dose prednisolone alone produced modest or no benefit. However, in the acute model, this drug combination potently inhibited TNF-a production (>50%) similar to a 100-fold higher dose of prednisolone alone (61%). In the chronic model, daily oral dosing of this drug combination significantly inhibited joint swelling by 64%, an inhibition equivalent to a >10-fold higher dose of prednisolone (51%) alone. Chronic treatment with this drug combination did not recapitulate the steroid toxicities on body and organ weight, blood glucose, and HPA suppression observed with high dose steroid treatment.

Combination Comprising Paroxetine and Prednisolone

In certain embodiments, the drug combination according to the present invention comprises paroxetine (a selective serotonin reuptake inhibitor (SSRI))and prednisolone (a steroid)

The structure of prednisolone is shown above. The structure of paroxetine is shown below:

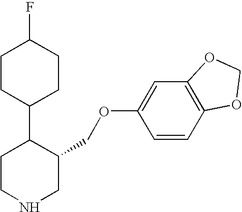

This drug combination is in clinical phase IIa trials in Europe and Canada.

Preclinical data suggest that when administered together, paroxetine synergistically increases the immuno-modulatory activity of a reduced-dose of prednisolone without a comparable increase in its adverse side effects, indicating that this drug combination may have a superior risk-to-benefit ratio compared to traditional steroids.

This drug combination elicits synergistic immuno-modulatory effects without potentiating steroid-associated side effects, and does so through paroxetine's action on key signaling pathways in activated T cells distinct from and synergistic with those affected by prednisolone. It synergistically inhibits multiple cytokines, including TNF-α, IFN-γ and IL-2, released from stimulated primary human lymphocytes.

Due to the mechanism of synergy of this drug combination, paroxetine does not promote glucocorticoid receptor trafficking or potentiate prednisolone's ability to transactivate a GRE reporter plasmid T cells. Paroxetine represses NFAT activation, translocation and transactivation and inhibits NFkB and AP 1 activation through inhibition of p38 and JNK but not ERK activation.

In an in vivo LPS-induced TNF-α release model, this drug combination inhibits TNF-α production by 51% when given 2 hours prior to LPS treatment. This effect was similar to a 100×higher dose of prednisolone alone. The anti-inflammatory effect in vivo was not accompanied by potentiation of steroid side effects such as HPA suppression.

This drug combination has been tested in a human pharmacology endotoxemia study, an acute model of inflammatory markers. In the study, this drug combination inhibited certain pro-inflammatory biomarkers, such as TNF-alpha, IL-6, and C-reactive protein and increased the anti-inflammatory cytokine IL-10.

Combination Comprising Dipyridamole and Prednisolone

In certain embodiments, the drug combination according to the present invention comprises dipyridamole (a cardiovascular drug, an anti-platelet agent) and prednisolone (a steroid).

The structure of prednisolone is shown above. The structure of dipyridamole is shown below:

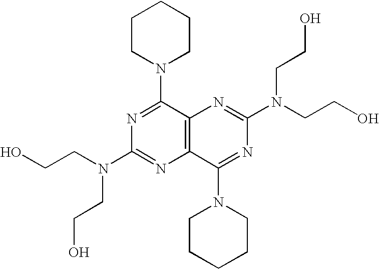

This drug combination is in clinical phase II trials in Europe.

Preclinical data suggest that when administered together, dipyridamole synergistically increases the immuno-modulatory activity of the reduced-dose prednisolone without a comparable increase in its adverse side effects, indicating that this may have a superior risk-to-benefit ratio compared to traditional steroids.

In vitro, this drug combination synergistically inhibits TNF-α release from stimulated primary human lymphocytes as measured by Loewe and other standard synergy models. This drug combination also synergistically inhibits IFN-γ in vitro. Although not wishing to be bound by any particular theories, it is believed that the increased activity of the reduced-dose steroid in this drug combination occurs in part through an action involving macrophages, which are important components of the immune system.

In vivo, a single p.o. dose of this drug combination potently inhibited LPS-induced TNF-α production by 72%. In the adjuvant model, this drug combination inhibited joint swelling by 54% while in the CIA model, the combination of dipyridamole and prednisolone reduced the arthritis severity score by 58%, compared to vehicle controls. In each model, the components of this drug combination had little or no activity. Further, the effect of this drug combination in these models was similar to that seen with ≧10 fold higher steroid doses. Chronic treatment with this drug combination did not recapitulate the steroid toxicities on body weight, glucose utilization and HPA suppression observed with high dose steroid treatment.

Combination Comprising Dexamethasone and Econazole

In certain embodiments, the drug combination according to the present invention comprises dexamethasone (a steroid) and econazole (an antifungal agent).

The structure of dexamethasone is shown below:

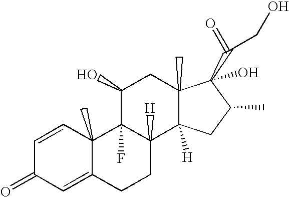

The structure of econazole nitrate is shown below:

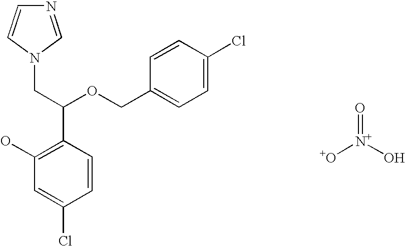

This drug combination is a research phase combination that has not yet entered preclinical studies. In vitro studies show this drug combination synergistically inhibits the production of TNF-α.

Combination Comprising Diflorasone and Alprostadil

In certain embodiments, the drug combination according to the present invention comprises diflorasone (a steroid) and alprostadil (a prostaglandin).

The structure of diflorasone is shown below:

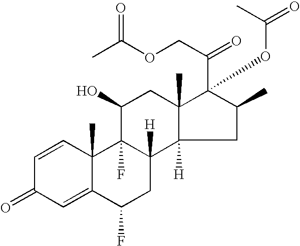

The structure of prostaglandin E is shown below:

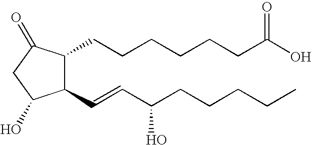

This drug combination synergistically inhibits multiple cytokines including TNF-α released from LPS-stimulated human peripheral mononuclear blood cells. It is a research phase combination that have not yet entered preclinical phase.

Combination Comprising Dipyridamole and Amoxapine

In certain embodiments, the drug combination of the present invention comprises dipyridamole (an anti-platelet agent) and amoxapine (an anti-depressant).

The structures of dipyridamole and amoxapine are shown above.

This drug combination is in clinical phase IIa trials in Europe.

This drug combination is an orally administered synergistic cytokine modulator that combines two active pharmaceutical ingredients, neither of which is indicated for the treatment of immuno-inflammatory disease. When administered together, these active pharmaceutical ingredients show the potential in preclinical studies to synergistically inhibit important disease-relevant cytokines, including the cytokine TNF-alpha.

This drug combination synergistically inhibits multiple cytokines including TNF-α released from LPS-stimulated human peripheral mononuclear blood cells. This affect was confirmed in the acute in vivo LPS model where the drug combination of dipyridamole and amoxapine significantly inhibited TNF-α release (>75%). This effect was similar to a high dose of prednisolone (10 mg/Kg). The components of this drug combination had no significant effect in the in vivo TNF-α release studies. In the chronic arthritis model, daily oral dosing of this drug combination significantly inhibited joint swelling by >40%. The components of this drug combination had minimal effects in this model. Furthermore, chronic treatment with this drug combination or its components elicited minimal effects on body and organ weight, blood glucose, and HPA suppression.

Combination Comprising Dipyridamole and Ibudilast

In certain embodiments, the drug combination of the present invention comprises dipyridamole (an anti-platelet agent) and ibudilast (a phosphodiesterase IV inhibitor).

The structure of ibudilast is shown below, while the structure of dipyridamole is shown above.

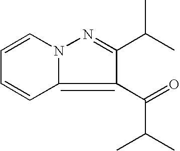

This drug combination is a research phase combination that has not yet entered preclinical studies. It synergistically inhibits TNF-α released from LPS-stimulated human peripheral mononuclear blood cells.

Combination Comprising Nortriptyline and Loratadine (or Desloratadine)

In certain embodiments, the drug combination according to the present invention comprises nortriptyline (a tricyclic anti-depressant agent) and loratadine (or desloratadine)(an antihistamine).

The structure of nortriptyline hydrochloride is shown below:

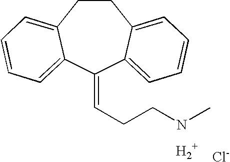

The structure of loratadine is shown below:

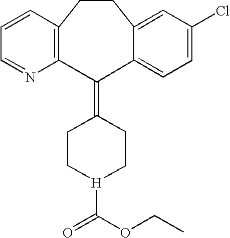

This drug combination is a research phase combination that has not yet entered preclinical studies.

This drug combination has shown potent synergistic inhibition of TNF-α and other pro-inflammatory cytokines in in vitro studies. In addition, loratadine inhibits mast cells and eosinophil activation.

Combination Comprising Albendazole and Pentamidine

In certain embodiments, the drug combination according to the present invention comprises albendazole and pentamidine.

The structure of albendazole is shown below:

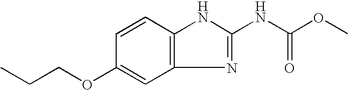

The structure of pentamidine is shown below:

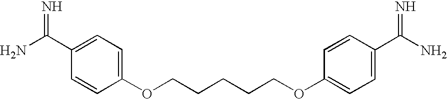

This drug combination synergistically inhibits the proliferation of A549 cells in vitro. It has demonstrated potent, highly synergistic anti-tumor effects in animal models of NSCLC. The anti-tumor effects of this drug combination are dose dependent and comparable to the activity of gold standard antineoplastics without the associated toxicities.

Combination Comprising Itraconazole and Lovastatin

In certain embodiments, the drug combination according to the present invention comprise itraconazole (an antifungal agent) and lovastatin (an HMG-CoA reductase inhibitor).

The structure of itraconazole is shown below:

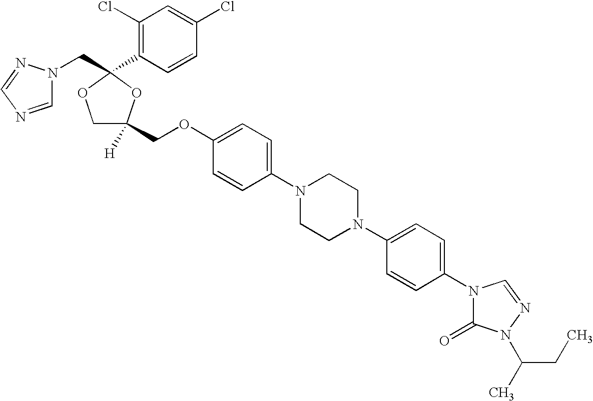

The structure of lovastatin is shown below:

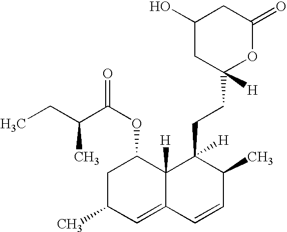

This drug combination demonstrates highly synergistic inhibition of the proliferation of multiple cancer cell lines in vitro, including A549 (NSCLC), PANC-1 (Pancreatic), HCT-116 (Colorectal), DU-145 (Prostate), and SKMEL28 (Melanoma). It has potential application to multiple proliferative diseases. This drug combination is in the research phase.

Combination Comprising Terbinafine and Manganese Sulfate

In certain embodiments, the drug combination according to the present invention comprises terbinafine (an anti-fungal agent) and manganese sulfate (to provide a metal ion).

The structure of terbinafine hydrochloride is shown below:

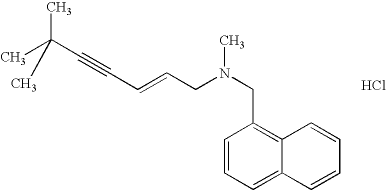

The structure of manganese sulfate is shown below:

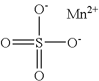

Manganese ion synergistically potentiates the antifungal activity of terbinafine against multiple drug-resistant strains of C. glabrata.

Drug Combination Comprising a Tricyclic Compound and a Steroid

In certain embodiments, the drug combination that has anti-scarring activity comprises at least two agents, wherein at least one agent is a tricyclic compound, such as a tricyclic antidepressant (TCA) and at least one second agent is a steroid such as a corticosteroid. Examples of drug combinations include a drug combination that comprises at least two agents in amounts that together may be sufficient to alter the immune response, that is, the at least two agents alone or in combination reduce or inhibit an immune response by a host or subject (or patient), including inhibiting or reducing inflammation (an inflammatory response) and/or an autoimmune response.

The drug combination may further comprise one or more additional compounds (e.g., a glucocorticoid receptor modulator, NSAID, COX-2 inhibitor, DMARD, biologic, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid). The composition may be formulated, for example, for topical administration or systemic administration.

Compounds useful in the drug combinations include those described herein in any of their pharmaceutically acceptable forms, including isomers such as diastereomers and enantiomers, salts, solvates, and polymorphs thereof, as well as racemic mixtures of the compounds described herein.

In the generic descriptions of compounds described herein, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

The term “pharmaceutically active salt” refers to a salt that retains the pharmaceutical activity of its parent compound.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “fexofenadine” is meant the free base, as well as any pharmaceutically acceptable salt thereof (e.g., fexofenadine hydrochloride).

Tricyclic Compound

By “tricyclic compound” is meant a compound having one of formulas (I), (II), (III), or (IV): 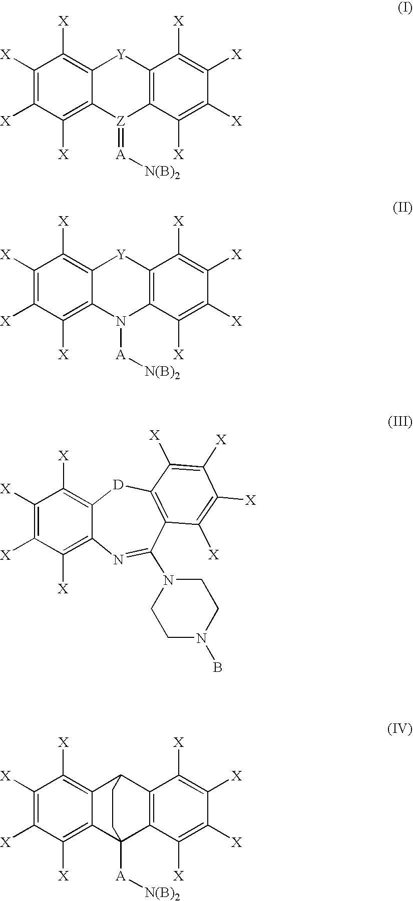
wherein each X is, independently, H, Cl, F, Br, I, CH₃, CF₃, OH, OCH₃, CH₂CH₃, or OCH₂CH₃;Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, or S(O)₀₋₂. In preferred embodiments, each X is, independently, H, Cl, or F; Y is (CH₂)₂, Z is C; A is (CH₂)₃; and each B is, independently, H, Cl, or F.

Tricyclic compounds include tricyclic antidepressants such as amoxapine, 8-hydroxyamoxapine, 7-hydroxyamoxapine, loxapine (e.g., loxapine succinate, loxapine hydrochloride), 8-hydroxyloxapine, amitriptyline, clomipramine, doxepin, imipramine, trimipramine, desipramine, nortriptyline, and protriptyline, although compounds need not have antidepressant activities to be considered tricyclic compounds as described herein.

Tricyclic compounds include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

Amoxapine

Amoxapine is a tricyclic antidepressant (TCA) of the dibenzoxapine type. It is structurally similar to the older TCAs and also shares similarities with the phenothiazines.

The exact action of TCAs is not fully understood, but it is believed that one of their important effects is the enhancement of the actions of norepinephrine and serotonin by blocking the reuptake of various neurotransmitters at the neuronal membrane. Amoxapine also shares some similarity with antipsychotic drugs in that it blocks dopamine receptors and can cause dyskinesia. Amoxapine also blocks the reuptake of norepinephrine, similar to the action of desipramine and maprotiline.

Based on the ability of amoxapine to act in concert with prednisolone to inhibit TNFα levels, one skilled in the art will recognize that other TCAs, as well as structural and functional analogs of amoxapine, can also be used in combination with prednisolone (or another corticosteroid—see below). Amoxapine analogs include, for example, 8-hydroxyamoxapine, 7-hydroxyamoxapine, loxapine, loxapine succinate, loxapine hydrochloride, 8-hydroxyloxapine, clothiapine, perlapine, fluperlapine, and dibenz(b,f)(1,4)oxazepine, 2-chloro-11-(4-methyl-1-piperazinyl)-, monohydrochloride.

Corticosteroids

By “corticosteroid” is meant any naturally occurring or synthetic compound characterized by a hydrogenated cyclopentanoperhydro-phenanthrene ring system and having immunosuppressive and/or antinflammatory activity. Naturally occurring corticosteriods are generally produced by the adrenal cortex. Synthetic corticosteroids may be halogenated. Functional groups required for activity include a double bond at Δ4, a C3 ketone, and a C20 ketone. Corticosteroids may have glucocorticoid and/or mineralocorticoid activity. Examples corticosteroids are provided herein.

In one embodiment, at least one (i.e.g, one or more) corticosteroid may be combined and/or formulated with a tricyclic compound in a drug combination described herein. Suitable corticosteroids include 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 1-dehydrocorticosterone; 11-deoxycortisol; 11-hydroxy-1,4-androstadiene-3,17- dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; dipropionate; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone; prednisolone 21-hemisuccinate; prednisolone 21-hemisuccinate free acid; prednisolone acetate; prednisolone famesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Prednisolone

Prednisolone, a synthetic adrenal corticosteroid, has anti-inflammatory properties, and is used in a wide variety of inflammatory conditions. It is desirable to reduce the amount of administered prednisolone because long-term use of steroids at can produce significant side effects.

Prednisolone is a member of the corticosteroid family of steroids. Based on the shared structural features and apparent mechanism of action among the corticosteroid family, one skilled in the art will recognize that other corticosteroids can be used in combination with amoxapine or an amoxapine analog to treat inflammatory disorders. Corticosteroids include, for example, the compounds listed herein.

The compounds described herein are also useful when formulated as salts. For example, amytriptiline, another tricyclic compound, has been formulated as a hydrochloride salt, indicating that amoxapine can be similarly formulated. Prednisolone salts include, for example, prednisolone 21-hemisuccinate sodium salt and prednisolone 21-phosphate disodium salt.

Other Compounds

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

Steroid Receptor Modulators

Steroid receptor modulators (e.g., antagonists and agonists) may be used as a substitute for or in addition to a corticosteroid in the drug combinations described herein. Thus, in one embodiment, the drug combination features the combination of a tricyclic compound and a glucocorticoid receptor modulator or other steroid receptor modulator.

Glucocorticoid receptor modulators that may used in the drug combinations described herein include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other compounds that may be used as a substitute for or in addition to a corticosteroid in the drug combinations include, but are not limited to, A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Non-steroidal Anti-inflammatory Drugs (NSAIDs)

In certain embodiments, the tricyclic compound of the drug combination may be administered in conjunction with one or more of non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin.

When a tricyclic compound is administered in combination with acetylsalicylic acid, the combination may also be effective in modulating an immune response (suppressing TNFα, IL-1, IL-2 or IFN-γ) in vitro. Accordingly, the combination of a tricyclic compound in combination with acetylsalicylic acid and their analogs may be more effective than either agent alone in modulating an immune, particularly an immune response mediated by TNFα, IL-1, IL-2, and/or IFN-γ.

Acetylsalicylic acid, also known by trade name aspirin, is an acetyl derivative of salicylic acid and has the following structural formula.

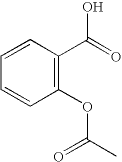

Aspirin is useful in the relief of headache and muscle and joint aches. Aspirin is also effective in reducing fever, inflammation, and swelling and thus has been used for treatment of rheumatoid arthritis, rheumatic fever, and mild infection. Thus in certain embodiments, a drug combination of a tricyclic compound and acetylsalicylic acid (aspirin) or an analog thereof can also be used in the devices and methods described herein.

An NSAID may be administered in conjunction with any one of the drug combinations described herein. For example, a drug combination that includes at least one drug that is also useful for treating and/or preventing an immunological disease or disorder, including an inflammatory disease or disorder, may be a combination of a tricyclic compound and a corticosteroid and further comprising an NSAID, such as acetylsalicylic acid, in conjunction with the combination described above.

Dosage amounts of acetylsalicylic acid are known to those skilled in medical arts, and generally range from about 70 mg to about 350 mg per day. When a lower or a higher dose of aspirin is needed, a formulation containing dipyridamole and aspirin may contain 0-25 mg, 25-50 mg, 50-70 mg, 70-75 mg, 75-80 mg, 80-85 mg, 85-90 mg, 90-95 mg, 95-100 mg, 100-150 mg, 150-160 mg, 160-250 mg, 250-300 mg, 300-350 mg, or 350-1000 mg of aspirin.

When the combinations described herein are used for treatment in conjunction with an NSAID, the dose of the individual components may be reduced substantially to a point below the doses that would be effective for achieving the same effects by administering NSAIDs (e.g., acetylsalicylic acid) or tricyclic compound alone or by administering a combination of an NSAID (e.g., acetylsalicylic acid) and a tricyclic compound. A drug combination that includes a tricyclic compound and an NSAID may have increased effectiveness, safety, tolerability, or satisfaction of treatment of a patient suffering from or at risk of suffering from inflammatory disorder or disease as compared to a composition having a tricyclic compound or an NSAID alone.

Nonsteroidal Immunophilin-dependent Immunosuppressants

In one embodiment, the drug combination comprises a tricyclic compound and a non-steroidal immunophilin-dependent immunosuppressant (NsIDI), optionally with a corticosteroid or other agent described herein.

By way of background, in healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus) and rapamycin target many types of immunoregulatory cells, including T-cells, and suppress the immune response in organ transplantation and autoimmune disorders.

In one embodiment, the NsIDI is cyclosporine, and in another embodiment, the NsIDI is tacrolimus. In another embodiment, the NsIDI is rapamycin and in still another embodiment, the NsIDI is everolimus. In still other embodiments, the NsIDI is pimecrolimus, or the NsIDI is a calcineurin-binding peptide. Two or more NsIDIs can be administered contemporaneously.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂-OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Tacrolimus

Tacrolimus (FK506) is an immunosuppressive agent that targets T cell intracellular signal transduction pathways. Tacrolimus binds to an intracellular protein FK506 binding protein (FKBP-12) that is not structurally related to cyclophilin (Harding et al., Nature 341:758-7601, 1989; Siekienka et al., Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of nuclear factor of activated T cells (NFAT), a nuclear component that initiates gene transcription required for proinflammatory cytokine (e.g., IL-2, gamma interferon) production and T cell activation. Thus, tacrolimus inhibits T cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 0.9% sodium chloride or 5% dextrose prior to injection.

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino 0-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages are provided below. By way of background, typically patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus, which is described further in detail herein, is the 33-epi-chloro derivative of the macrolactam ascomyin. Pimecrolimus structural and functional analogs are described in U.S. Pat. No. 6,384,073. Pimecrolimus is particularly useful for the treatment of atopic dermatitis.

Rapamycin

Rapamycin is a cyclic lactone produced by Streptomyces hygroscopicus. Rapamycin is an immunosuppressive agent that inhibits T cell activation and proliferation. Like cyclosporines and tacrolimus, rapamycin forms a complex with the immunophilin FKBP-12, but the rapamycin-FKBP-12 complex does not inhibit calcineurin phosphatase activity. The rapamycin immunophilin complex binds to and inhibits the mammalian kinase target of rapamycin (mTOR). mTOR is a kinase that is required for cell-cycle progression. Inhibition of mTOR kinase activity blocks T cell activation and proinflammatory cytokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcineurin inhibitors, these agents are useful in the methods of the invention.

As described herein, in one embodiment, a drug combination comprises a tricyclic compound and a corticosteroid. In certain specific embodiments, the drug combination comprises a tricyclic compound wherein the tricyclic compound is a tricyclic antidepressant selected from amoxapine, 8-hydroxyamoxapine, 8-methoxyloxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, maprotiline, norclozapine, olanzapine, or protriptyline. In a specific embodiment, the tricyclic compound is amoxapine.

In a particular embodiment, the tricyclic compound is combined with a corticosteroid wherein the corticosteroid is dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, or triamcinolone acetonide 21-palmitate.

In a certain specific embodiment, the corticosteroid is prednisolone. In one embodiment, the drug combination comprises amoxapine and prednisolone. In other specific embodiments, the corticosteroid is prednisolone and the tricyclic compound is protriptyline; in another specific embodiment the corticosteroid is prednisolone and the tricyclic compound is nortriptyline. In other specific embodiments, the drug combination comprises prednisolone and maprotaline. In certain specific embodiments, the corticosteroid is prednisolone and the tricyclic compound is loxapine; the corticosteroid is prednisolone and the tricyclic compound is desipramine; the corticosteroid is prednisolone and the tricyclic compound is clomipramine; the corticosteroid is prednisolone and the tricyclic compound is protriptyline. In another embodiment, the drug combination comprises prednisolone and fluoxotine; in still another embodiment, the drug combination comprises prednisolone and norclozapine.

In other embodiments, the drug combination comprises budesonide and amitriptyline; dexamethasone and amitriptyline; diflorasone and amitriptyline; hydrocortisone and amitriptyline; prednisolone and amitriptyline; triamcinolone and amitriptyline; budesonide and amoxapine; dexamethasone and amoxapine; betamethasone and amoxapine; hydrocortisone and amoxapine; triamcinolone and amoxapine; betamethasone and clomipramine; budesonide and clomipramine; dexamethasone and clomipramine; diflorasone and clomipramine; hydrocortisone and clomipramine; triamcinolone and clomipramine. In other embodiments, the drug combination comprises desipramine with any one of betamethasone, budesonide, dexamethasone, diflorasone, hydrocortisone, prednisolone, and triamcinolone. In still other specific embodiments, the drug combination comprises imipramine with any one of betamethasone, budesonide, dexamethasone, diflorasone, hydrocortisone, prednisolone, and triamcinolone. In another specific embodiment, the drug combination comprises nortriptyline and any one of betamethasone, budesonide, dexamethasone, hydrocortisone, prednisolone, and triamcinolone. In another embodiment, the drug combination comprises protriptyline and any one of betamethasone, budesonide, dexamethasone, diflorasone, hydrocortisone, prednisolone, and triamcinolone.

In another specific embodiment, a structural analog of amoxapine may be used in the drug combination. Such a structural analog may include clothiapine, perlapine, fluperlapine, or dibenz (b,f)(1,4)oxazepine, 2-chloro-11-(4-methyl-1-piperazinyl)-, monohydrochloride, which may be combined with a corticosteroid for use in the devices and methods described herein.

In other certain specific embodiments, the drug combination comprises a tricyclic compound wherein the tricyclic compound is amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, or trimipramine. In a particular embodiment, the tricyclic compound is combined with a corticosteroid, which in certain embodiments is prednisolone, cortisone, budesonide, dexamethasone, hydrocortisone, methylprednisolone, fluticasone, prednisone, triamcinolone, or diflorasone. In a certain specific embodiment, the tricyclic compound is nortriptyline and the corticosteroid is budesonide. The compositions may further comprise an NSAID, COX-2 inhibitor, biologic, DMARD, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid. In a specific embodiment, the NSAID is ibuprofen, diclofenac, or naproxen. In another specific embodiment, the COX-2 inhibitor is rofecoxib, celecoxib, valdecoxib, or lumiracoxib. In other certain embodiments, the biologic is adelimumab, etanercept, infliximab, CDP-870, rituximab, or atlizumab; and in other specific embodiments, DMARD is methotrexate or leflunomide; a xanthine is theophylline; a beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline; a non-steroidal immunophilin-dependent immunosuppressant is cyclosporine, tacrolimus, pimecrolimus, or ISAtx247; a vitamin D analog is calcipotriene or calcipotriol; a psoralen is methoxsalen; a retinoid is acitretin or tazoretene; a 5-amino salicylic acid is mesalamine, sulfasalazine, balsalazide disodium, or olsalazine sodium; and a small molecule immunomodulator is VX 702, SCIO 469, doramapimod, RO 30201195, SCIO 323, DPC 333, pranalcasan, mycophenolate, or merimepodib.

Drug Combination Comprising a Tetra-Substituted Pyrimidopyrimidine and a Corticosteroid

In another embodiment, the drug combination that has anti-scarring activity comprises a tetra-substituted pyrimidopyrimidine, such as dipyridamole (also known as 2,6-bis(diethanolamino)-4,8-dipiperidinopyrimido(5,4-d)pyrimidine), and a corticosteroid, such as fludrocortisone (as known as 9-alpha-fluoro-11-beta, 17-alpha, 21-trihydroxy-4-pregnene-3,20-dione acetate) or prednisolone (also known as 1-dehydrocortisol; 1-dehydrohydrocortisone; 1,4-pregnadiene-11beta, 17alpha,21-triol-3,20-dione; and 11beta, 17alpha,21-trihydroxy-1,4-pregnadiene-3,20-dione). At least one biological activity of such agents is the capability to substantially suppress TNFα levels induced in peripheral blood mononuclear cells (PBMCs). Thus, such a drug combination also has the capability to alter the immune response, including inhibiting or reducing inflammation (i.e., an inflammatory response) and/or an autoimmune response.

An exemplary composition comprises (i) a corticosteroid and (ii) a tetra-substituted pyrimidopyrimidine. An exemplary tetra-substituted pyrimidopyrimidine has structure of the formula (V): 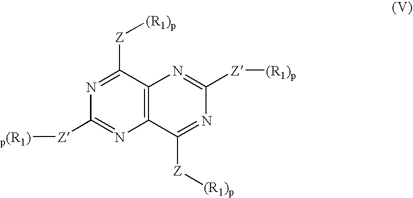
wherein each Z and each Z′ is, independently, N, O, C,

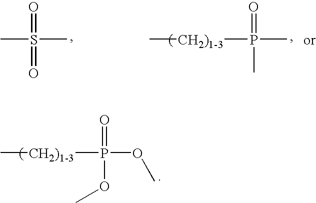

When Z or Z′ is O or 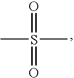
then p=1, when Z or Z′ is N, 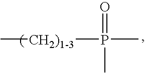 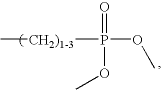
then p=2, and when Z or Z′ is C, then p=3. In formula (V), each R₁ is, independently, X; OH; N-alkyl (wherein the alkyl group has 1 to 20 carbon atoms); a branched or unbranched alkyl group having 1 to 20 carbon atoms; or a heterocycle. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusively. Each Y is, independently, H, F, Cl, Br, or I. In one embodiment, each Z is the same moiety, each Z′ is the same moiety, and Z and Z′ are different moieties. The two compounds are each administered in an amount that, when combined with the second compound, is sufficient to treat or prevent the immunoinflammatory disorder.

The drug combination may also suppress production of one or more proinflammatory cytokines in a host or subject to whom the device is administered, wherein the device comprises an implant and a drug combination as described herein and wherein the drug combination comprises (i) a corticosteroid; and (ii) a tetra-substituted pyrimidopyrimidine having formula (V).

In particularly useful tetra-substituted pyrimidopyrimidines, R₁ is a substituted or unsubstituted furan, purine, or pyrimidine, (CH₂CH₂OY), (CH₂CH(OH)CH₂OY), (HCH₂CH(OH)CX₃), ((CH₂)ₙOY), where n=2-5,

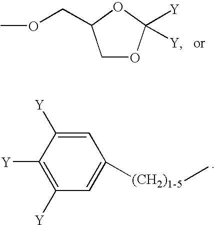

In other useful tetra-substituted pyrimidopyrimidines, each Z is N and the combination of the two associated R₁ groups is —(CH₂)₅—, and each Z′ is N and each associated R₁ group is —CH₂CH₂OH.

The tetra-substituted pyrimidopyrimidine and the corticosteroid may also be combined with a pharmaceutically acceptable carrier, diluent, or excipient.

In certain embodiments, a drug combination comprises one or more tetra-substituted pyrimidopyrimidine compounds and one or more corticosteroid compounds. The drug combination may feature higher order combinations of tetra-substituted pyrimidopyrimidines and corticosteroids. Specifically, one, two, three, or more tetra-substituted pyrimidopyrimidines may be combined with one, two, three, or more corticosteroids. In certain embodiments, the tetra-substituted pyrimidopyrimidine, the corticosteroid, or both are approved by the United States Food and Drug Administration (USFDA) for administration to a human.

Exemplary tetra-substituted pyrimidopyrimidines that may be used in the drug combinations described herein include, for example, 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine), mopidamole, dipyridamole monoacetate, NU3026 (2,6-di-(2,2-dimethyl-1,3-dioxolan-4-yl)-methoxy-4,8-di-piperidinopyrimidopyrimidine), NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), and NU3076 (2,6-bis(diethanolamino)-4,8-di-4-methoxybenzylaminopyrimidopyrimidine).

Dipyridamole

Dipyridamole (2,6-bis(diethanolamino)-4,8-dipiperidinopyrimido(5,4-d)pyrimidine) is a tetra-substituted pyrimidopyrimidine that is used as a platelet inhibitor, e.g., to prevent blood clot formation following heart valve surgery and to reduced the moribundity associated with clotting disorders, including myocardial and cerebral infarction.

Exemplary tetra-substituted pyrimidopyrimidines are 2,6-disubstituted 4,8-dibenzylaminopyrimido[5,4-d]pyrimidines, including, for example, mopidamole, dipyridamole monoacetate, NU3026 (2,6-di-(2,2-dimethyl-1,3-dioxolan-4-yl)-methoxy-4,8-di-piperidinopyrimidopyrimidine), NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), and NU3076 (2,6-bis(diethanolamino)-4,8-di-4-methoxybenzylaminopyrimido-pyrimidine) (see, e.g., Curtin et al., Br. J. Cancer 80:1738-1746, 1999).

In a particular embodiment, the tetra-substituted pyrimidopyrimidine compound is a 2,6-disubstituted 4,8-dibenzylaminopyrimido[5,4-d]pyrimidine. In another particular embodiment, the compound is dipyridamole, mopidamole, dipyridamole monoacetate, NU3026 (2,6-di-(2,2-dimethyl-1,3-dioxolan-4-yl)-methoxy-4,8-di-piperidinopyrimidopyrimidine) NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), or NU3076 (2,6-bis(diethanolamino)-4,8-di-4-methoxybenzylaminopyrimidopyrimidine), and in a specific embodiment, the compound is dipyridamole. In another particular embodiment, tetra-substituted pyrimidopyrimidine compound is a 2,6-disubstituted 4,8-dibenzylaminopyrimido[5,4-d]pyrimidine, and in another particular embodiment, compound is dipyridamole, mopidamole, dipyridamole monoacetate, NU3026, NU3059, NU3060, or NU3076.

Corticosteroids

As described herein, by “corticosteroid” is meant any naturally occurring or synthetic steroid hormone that can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at Δ4, a C3 ketone, and a C20 ketone. Corticosteroids may have glucocorticoid and/or mineralocorticoid activity. In certain embodiments, the corticosteroid is either fludrocortisone or prednisolone. Additional exemplary corticosteroids are provided in detail herein and are known in the art.

In certain embodiments, the drug combination comprises at least one of the corticosteroids: fludrocortisone (also as known as 9-alpha-fluoro-11-beta, 17-alpha, 21-trihydroxy-4-pregnene-3,20-dione acetate) and prednisolone (also known as 1-dehydrocortisol; 1-dehydrohydrocortisone; 1,4-pregnadiene-11beta, 17alpha, 21-triol-3,20-dione; and 11beta, 17alpha, 21-trihydroxy-1,4-pregnadiene-3,20-dione); however, a skilled artisan will recognize that structural and functional analogs of these corticosteroids can also be used in combination with the tetra-substituted pyrimidopyrimidines in the methods and compositions described herein. Other useful corticosteroids may be identified based on the shared structural features and apparent mechanism of action among the corticosteroid family. Other exemplary corticosteroids are described in greater detail herein.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

In another embodiment, the corticosteroid is algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, or wortmannin.

By “heterocycle” is meant any cyclic molecule, wherein one or more of the ring atoms is an atom other than carbon. Preferable heterocycles consist of one or two ring structures. Preferable heteroatoms are N, O, and S. Each ring structure of the heterocycle consists of 3-10 atoms, preferably 4-8 atoms, and most preferably 5-7 atoms. Each ring structure need not contain a heteroatom, provided that a heteroatom is present in at least one ring structure. Preferred heterocycles are, for example, beta-lactams, furans, tetrahydrofurans, pyrroles, pyrrolidines, thiophenes, tetrahydrothiophenes, oxazoles, imidazolidine, indole, guanine, and phenothiazine.

By the term “cytokine suppressing amount” is meant an amount of the combination which will cause a decrease in the vivo presence or level of the proinflammatory cytokine, when given to a patient for the prophylaxis or therapeutic treatment of an immunoinflammatory disorder which is exacerbated or caused by excessive or unregulated proinflammatory cytokine production.

The combination of a tetra-substituted pyrimidopyrimidine with a corticosteroid has substantial TNFα suppressing activity against stimulated white blood cells. The combinations of dipyridamole with fludrocortisone, and dipyridamole with prednisolone were particularly effective. Thus, the combination of a tetra-substituted pyrimidopyrimidine with a corticosteroid may also be useful for inhibiting an immune response, particularly an inflammatory response.

In a specific embodiment, the drug combination comprises dipyridamole and fludrocortisone. In another specific embodiment, the drug combination comprises dipyridamole and prednisolone. In yet another specific embodiment, the drug combination comprises dipyridamole and prednisone.

Drug Combination Comprising a Prostaglandin and a Retinoid

In another embodiment, the drug combination that has anti-scarring activity comprises at least two agents wherein at least one agent is a prostaglandin, such as alprostadil (also known as prostaglandin E1; (11α, 13E, 15S)-11,15-dihydroxy-9-oxoprost-13-enoic acid; 11α, 15α-dihydroxy-9-oxo-13-trans-prostenoic acid; or 3-hydroxy-2-(3-hydroxy-1-octenyl)-5-oxocyclopentaneheptanoic acid), and at least one second agent is a retinoid, such as tretinoin (also known as vitamin A; all trans retinoic acid; or 3,7-dimethyl-9-(2,6,6-trimethylcyclohex-1-enyl)nona-2,4,6,8-all-trans-tetraenoic acid). These compounds also exhibit the capability to substantially suppress TNFα levels induced in white blood cells. TNFα is a major mediator of inflammation.

Exemplary prostaglandin compounds include but are not limited to alprostidil, dinoprostone, misoprostil, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α, and prostaglandin J.

By “retinoid” is meant retinoic acid, retinol, and retinal, and natural or synthetic derivatives of retinoic acid, retinol, or retinal that are capable of binding to a retinoid receptor and consist of four isoprenoid units joined in a head-to-tail manner. Examples of retinoids include tretinoin, vitamin A2 (3,4-didehydroretinol), α-vitamin A (4,5-didehydro-5,6-dihydroretinol), 13-cis-retinol, 13-cis retinoic acid (isotretinoin), 9-cis retinoic acid (9-cis-tretinoin), 4-hydroxy all-trans retinoic acid, torularodin, methyl retinoate, retinaldehyde, 13-cis-retinal, etretinate, tazoretene, acetretin, alitretinoin and adapelene.

In certain embodiments, the composition comprises a prostaglandin and a retinoid wherein the prostaglandin is alprostidil, misoprostil, dinoprostone, prostaglandin E2, prostaglandin A1, prostaglandin A2, prostaglandin B1 , prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α or prostaglandin J. In certain specific embodiments, the prostaglandin is alprostadil or misoprostil. In certain embodiments, the retinoid is retinoid is tretinoin, retinal, retinol, vitamin A2, α-vitamin A, 13-cis-retinol, isotretinoin, 9-cis-tretinoin, 4-hydroxy all-trans retinoic acid, torularodin, methyl retinoate, retinaldehyde, 13-cis-retinal, etretinate, tazoretene, acetretin, alitretinoin or adapelene. In a specific embodiment, the retinoid is tretinoin or retinol. In one specific embodiment, the prostaglandin is alprostidil and the retinoid is tretinoin or retinol.

Drug Combination Comprising an Azole and a Steroid

In another embodiment, the drug combination that has anti-scarring activity comprises at least two agents wherein at least one agent is an azole, and at least one second agent is a steroid. A combination of an azole and a steroid also is capable of substantially suppressing TNF-α levels induced in white blood cells and has anti-inflammatory activity (i.e., reduces an immune response). In one embodiment, the azole is an imidazole or a triazole and the steroid is a corticosteroid, such as a glucocorticoid or a mineralocorticoid.

The azole/steroid combinations result in the unexpected enhancement of the steroid activity by as much as 10-fold when steroid is combined with a subtherapeutic dose of an azole, even when the azole is administered at a dose lower than that known to be effective as an antifungal agent. For example, ketoconazole is often administered at 200 mg/day orally and reaches a serum concentration of about 3.2 micrograms, while prednisone is generally administered in amounts between 5-200 mg. A 10-fold increase in the potency of the steroid can be achieved by combining it at 5 mg/day with 100 mg ketoconazole. The specific amounts of the azole (e.g., an imidazole or a triazole) and a steroid (e.g., a corticosteroid, such as a glucocorticoid or a mineralocorticoid) in the drug combination depend on the specific combination of components (i.e., the specific azole/steroid combination) and can be determined by one skilled in the art.

The azole may be selected from an imidazole or a triazole. In certain embodiments, the imidazole is selected from sulconazole, miconazole, clotrimazole, oxiconazole, butocontazole, tioconazole, econazole, and ketoconazole. In other certain embodiments, the triazole is selected from itraconazole, fluconazole, voriconazole, posaconazole, ravuconazole, and terconazole.

In certain embodiments, the drug combination comprises an azole selected from sulconazole, miconazole, clotrimazole, oxiconazole, butocontazole, tioconazole, econazole, and ketoconazole, or itrazonazole, fluconazole, voriconazole, posaconazole, ravuconazole, and terconazole, and a second compound is selected from dexamethasone, hydrocortisone, methylprednisolone, prednisone, traimcinolone, and diflorasone.

By “azole” is meant any member of the class of anti-fungal compounds having a five-membered ring of three carbon atoms and two nitrogen atoms (e.g., the imidazoles) or two carbon atoms and three nitrogen atoms (e.g., triazoles), which are capable of inhibiting fungal growth. A compound is considered “antifungal” if it inhibits growth of a species of fungus in vitro by at least 25%. Typically, azoles are administered in dosages of greater than 200 mg per day when used as an antifungal agent. Exemplary azoles for use in the invention are described herein.

Antifungal azoles (e.g., imidazoles and triazoles) as described herein refer to any member of the class of anti-fungal compounds having a five-membered ring of three carbon atoms and two nitrogen atoms (imidazoles) or two carbon atoms and three nitrogen atoms (triazoles). Exemplary azoles are described above.

As previously described herein by “corticosteroid” is meant any naturally occurring or synthetic steroid hormone that can be derived from cholesterol and is characterized by a hydrogenated cyclopentanoperhydrophenanthrene ring system. Naturally occurring corticosteriods are generally produced by the adrenal cortex. Synthetic corticosteriods may be halogenated. Functional groups required for activity include a double bond at Δ4, a C3 ketone, and a C20 ketone. Corticosteroids may have glucocorticoid and/or mineralocorticoid activity. Examples of exemplary corticosteroids are described above.

Corticosteroids are described in detail herein and refer to a class of adrenocortical hormones that include glucocorticoids, mineralocorticoids, and androgens, which are derived from cholesterol and is characterized by a hydrogenated cyclopentanoperhydrophenanthrene ring system. Exemplary corticosteroids are described herein and include, for example, budesonide and analogs of budesonide (e.g., budesonide (11-beta, 16-alpha(R)), budesonide (11-beta, 16-alpha(S)), flunisolide, desonide, triamcinolone acetonide, halcinonide, flurandrenolide, fluocinolone acetonide, triamcinolone hexacetonide, triamcinolone diacetate, flucinonide, triamcinolone, amcinafal, deflazacort, algestone, procinonide, flunisolide, hyrcanoside, descinolone, wortmannin, formocortal, tralonide, flumoxonide, triamcinolone acetonide 21-palmitate, and flucinolone, desonide, dexamethasone, desoximetasone, betamethasone, fluocinolide, triamcinolone, triamcinolone acetonide, triamcinolone diacetate, triamcinolone hexacetonide, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, fludrocortisone, paramethasone acetate, prednisolone, prednisone, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone-21-acetate, betamethasone-17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone-21-hemisuccinate free acid, prednisolone-21-acetate, prednisolone-21(-beta-D-glucuronide), prednisolone metasulphobenzoate, prednisolone terbutate, 6-alpha-methylprednisolone, 6-alpha-methylprednisolone 21-hemisuccinate sodium salt, 6-alpha-fluoroprednisolone, 6-alpha-methylprednisolone 21-acetate, 6-alpha,9-alpha-difluoroprednisolone 21-acetate 17-butyrate, prednisolone metasulphobenzoate, cortodoxone, isoprednidene, 21-deoxycortisol, prednylidene, deprodone, 6-beta-hydroxycortisol, and triamcinolone acetonide-21-palmitate. In certain embodiments, the corticosteroid is selected from cortisone, dexamethasone, hydrocortisone, methylprenisolone, prednisone, traimcinolone, and diflorasone.

In certain embodiments, the corticosteroid is a glucocorticoid or a mineralocorticoid, and the azole is an imidazole, which is selected sulconazole, miconazole, clotrimazole, oxiconazole, butocontazole, tioconazole, econazole, and ketoconazole. In another embodiment, the azole is an itrazonazole and is selected from sulconazole, miconazole, clotrimazole, oxiconazole, butocontazole, tioconazole, econazole, and ketoconazole. In another embodiment, the azole is a triazole is selected from itrazonazole, fluconazole, voriconazole, posaconazole, ravuconazole, and terconazole. In one embodiment, the corticosteroid is a glucocorticoid selected from cortisone, dexamethasone, hydrocortisone, methylprednisolone, prednisone, traimcinolone, and diflorasone. In certain embodiments, the drug combination comprises an azole compound selected from sulconazole, miconazole, clotrimazole, oxiconazole, butocontazole, tioconazole, econazole, and ketoconazole, or itrazonazole, fluconazole, voriconazole, posaconazole, ravuconazole, and terconazole; and comprises a steroid selected from dexamethasone, hydrocortisone, methylprednisolone, prednisone, traimcinolone, and diflorasone. In one specific embodiment, the drug combination comprises dexamethasone and econazole, and in another specific embodiment, the drug combination comprises diflorasone and clotrimazole.

In another particular embodiment, the drug combination comprises an azole and a steroid, with the proviso that the amount of the azole present in the composition is not sufficient for the composition to be administered as an effective antifungal agent. In a preferred embodiment, the azole and steroid are present in amounts in which the activity of the steroid is enhanced at least 10-fold by the presence of the azole. In another certain embodiment, the ratio of azole to steroid (e.g., fluconazole to glucocorticoid) is about 50:1 by weight, more desirably at least about 20:1 or 10:1 by weight, and most desirably about 4:1, 2:1, or 1:1 by weight.

Compounds useful for drug combinations described herein include those described herein in any of their pharmaceutically acceptable forms, including isomers such as diastereomers and enantiomers, salts, solvates, and polymorphs thereof, as well as racemic mixtures of the compounds described herein.

Drug Combination Comprising a Steroid and (A) a Protaglandin; (B) a Beta-Adrenergic Receptor Ligand; (C) an Anti-Mitotic Agent; or (D) a Microtubule Inhibitor; and Other Combinations Thereof

In one embodiment, a drug combination that has anti-scarring activity comprises at least two agents wherein at least one agent is a steroid and at least one second agent is selected from a prostaglandin, a beta-adrenergic receptor ligand, an anti-mitotic agent, and a microtubule inhibitor. In other embodiments, the drug combination comprises an anti-mitotic agent, such as an azole, and a microtubule inhibitor.

In particular embodiments, a drug combination comprises a steroid and a prostaglandin wherein the prostaglandin is alprostadil and the steroid is diflorasone, prednisolone, or dexamethasone. In another embodiment, the drug combination comprises a beta-adrenergic receptor ligand and a steroid. In still another embodiment, an anti-mitotic agent such as podofilox (podophyllotoxin) is combined with a steroid (such as diflorasone, prednisolone, or dexamethasone)

In certain embodiments, the drug combination comprises a microtubule inhibitor (e.g., colchicine and vinblastine) and a steroid such as diflorasone, prednisolone, or dexamethasone. In yet another embodiment a microtubule inhibitor (e.g., colchicine and a vinca alkaloid (e.g., vinblastine)) is combined with an anti-mitotic agent that is an azole (e.g., clotrimazole). For example vinblastine can be used in combination with clotrimazole. Additional drug combinations comprise one or more of the compounds described above (i.e., a prostaglandin, a beta-adrenergic receptor ligand, an anti-mitotic agent, or a microtubule inhibitor in combination with a steroid, and a microtubule inhibitor in combination with an azole) include in particular embodiments, for example, a prostaglandin that is alprostidil and a steroid that is diflorasone; a beta-adrenergic receptor ligand that is isoproterenol and a steroid that is prednisolone; an anti-mitotic agent that is podofilox and a steroid that is dexamethasone; a microtubule inhibitor that is colchicine and a steroid that is flumethasone; and a microtubule inhibitor that is vinblastine and an anti-mitotic agent that is the azole, clotrimazole.

A drug combination comprising at least one steroid and at least one of a prostaglandin, beta-adrenergic receptor ligand, anti-mitotic agent or microtubule inhibitor has the capability to substantially suppress TNFα levels induced in white blood cells. TNFα is a major mediator of inflammation. Specific blockade of TNFα by using antibodies that specifically bind to TNFα or by using soluble receptors is a potent treatment for patients having an inflammatory disease. Moreover, based on the shared action among prostaglandin family members, among beta-adrenergic receptor ligand family members, among anti-mitotic agent family members, among microtubule inhibitor family members, and among steroid family members, any member of each family can be replaced by another member of that family in the combination.

In addition, the combination of a microtubule inhibitor with an azole also provides substantial suppression of TNFα levels induced in white blood cells. Thus, this drug combination can similarly be used to reduce an immune response, such as inhibit or reduce an inflammatory response (or inflammation). Based on the shared action among microtubule inhibitor family members and azole family members, one member of a family can be replaced by another member of that family in the combination.

In certain embodiments, the drug combination has certain dose combinations, for example, the ratio of prostaglandin (e.g., alprostadil) to steroid (e.g., diflorasone) may be 10:1 to 20:1 by weight; the ratio of beta-adrenergic receptor ligand (e.g., isoproterenol) to steroid (e.g., prednisolone, glucocorticoid, mineralocorticoid) may be 10:1 to 100:1 by weight; the ratio of anti-mitotic agent (e.g., podofilox) to steroid (e.g., dexamethasone) may be 10:1 to 500:1 by weight; the ratio of microtubule inhibitor (e.g., colchicine) to steroid (e.g., flumethasone) may be 50:1 to 1000:1 by weight; and the ratio of microtubule inhibitor (e.g., vinblastine) to azole (e.g., clotrimazole) may be 2:1 to 1:2 by weight.

Compounds useful in the drug combinations described herein include those described herein in any of their pharmaceutically acceptable forms, including isomers such as diastereomers and enantiomers, salts, solvates, and polymorphs thereof, as well as racemic mixtures of the compounds described herein.

By “anti-mitotic agent” is meant an agent that is capable of inhibiting mitosis. Exemplary anti-mitotic agents include, for example, podofilox, etoposide, teniposide, and griseofulvin.

By “azole” is meant any member of the class of anti-fungal compounds having a five-membered ring of three carbon atoms and two nitrogen atoms (e.g., the imidazoles) or two carbon atoms and three nitrogen atoms (e.g., triazoles), which are capable of inhibiting fungal growth. A compound is considered “antifungal” if it inhibits growth of a species of fungus in vitro by at least 25%. Typically, azoles are administered in dosages of greater than 200 mg per day when used as an antifungal agent. The azole can be selected from an imidazole or a triazole. Examples of exemplary imidazoles include but are not limited to sulconazole, miconazole, clotrimazole, oxiconazole, butocontazole, tioconazole, econazole, and ketoconazole. Examples of exemplary triazoles include but are not limited to itraconazole, fluconazole, voriconazole, posaconazole, ravuconazole, and terconazole.

By “beta-adrenergic receptor ligand” is meant an agent that binds the beta-adrenergic receptor in a sequence-specific manner. Exemplary beta-adrenergic receptor ligands include agonists and antagonists. Exemplary beta-adrenergic receptor agonists include, for example, isoproterenol, dobutamine, metaproterenol, terbutaline, isoetharine, finoterol, formoterol, procaterol, ritodrine, salmeterol, bitolterol, pirbuterol, albuterol, levalbuterol, epinephrine, and ephedrine. Exemplary beta-adrenergic receptor antagonists include, for example, propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, and propafenone.

By “microtubule inhibitor” is meant an agent that is capable of affecting the equilibrium between free tubulin dimers and assembled polymers. Exemplary microtubule inhibitors include, for example, colchicine, vinca alkaloids (e.g., vinblastine, vincristine, vinorelbine, and vindesine), paclitaxel, and docetaxel.

By “prostaglandin” is meant a member of the lipid class of biochemicals that belongs to a subclass of lipids known as the eicosanoids, because of their structural similarities to the C-20 polyunsaturated fatty acids, the eicosaenoic acids. Exemplary prostaglandins include alprostidil, dinoprostone, misoprostil, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α, and prostaglandin J.

By “steroid” is meant any naturally occurring or synthetic hormone that can be derived from cholesterol and is characterized by a hydrogenated cyclopentanoperhydrophenanthrene ring system. Naturally occurring steroids are generally produced by the adrenal cortex. Synthetic steriods may be halogenated. Steroids may have corticoid, glucocorticoid, and/or mineralocorticoid activity. Examples of steroids are algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin, and other corticosteroids and steroids described herein. Desirably, the corticosteroid is selected from cortisone, dexamethasone, hydrocortisone, methylprednisolone, prednisone, traimcinolone, and diflorasone.

Accordingly in certain embodiments, a drug combination comprises a prostaglandin and a steroid, and in certain particular embodiments, the prostaglandin is alprostidil, misoprostil, dinoprostone, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α, or prostaglandin J. In a particular embodiment, the prostaglandin is alprostidil. In a more specific embodiment, the prostaglandin is alprostidil and the steroid is diflorasone.

In another embodiment, the composition comprises beta-adrenergic receptor ligand and a steroid, and in particular embodiments, the beta-adrenergic receptor ligand is isoproterenol, dobutamine, metaproterenol, terbutaline, isoetharine, finoterol, formoterol, procaterol, ritodrine, salmeterol, bitolterol, pirbuterol, albuterol, levalbuterol, epinephrine, ephedrine, propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, or propafenone. In a certain specific embodiment, the beta-adrenergic receptor ligand is isoproterenol. In another specific embodiment, the beta-adrenergic receptor ligand is isoproterenol and the steroid is prednisolone.

In still another embodiment, a composition comprises anti-mitotic agent and a steroid, wherein in certain embodiments, the anti-mitotic agent is podofilox, etoposide, teniposide, or griseofulvin. In a more specific embodiment, the antimitotic agent is podofilox. In another specific embodiment, the anti-mitotic agent is podofilox and the steroid is dexamethasone.

In other embodiment, the composition comprises a microtubule inhibitor and a steroid, and in specific embodiments, the microtubule inhibitor is an alkaloid, paclitaxel, or docetaxel, and wherein the alkaloid is colchicine or a vinca alkaloid. In certain embodiments, the vinca alkaloid is vinblastine, vincristine, vinorelbine, or vindesine. In other certain embodiments, the microtubule inhibitor is colchicine and said steroid is dexamethasone. In another specific embodiment, the microtubule inhibitor is colchicine and the steroid is flumethasone.

According to all the above embodiments, the steroid may be selected from dexamethasone, diflorasone, flumethasone, or prednisolone.

In another embodiment, the drug compound comprises a microtubule inhibitor and an azole, and in particular embodiments, the microtubule inhibitor is vinblastine, vincristine, vinorelbine, or vindesine. In another particular embodiment, the microtubule inhibitor is vinblastine. In another specific embodiment, the microtubule inhibitor is vinblastine and said azole is clotrimazole. In one embodiment, the azole is an imidazole or a triazole. In specific embodiments, the imidazole is selected from suconazole, miconazole, clotrimazole, oxiconazole, butoconazole, tioconazole, econazole, and ketoconazole. In another specific embodiment, the imidazole is clotrimazole. In a specific embodiment, the triazole is selected from itraconazole, fluconazole, voriconazole, posaconazole, ravuconazole, and terconazole. In one specific embodiment, the microtubule inhibitor is vinblastine and the azole is clotrimazole

For the drug combinations that comprise a steroid, the steroid is selected from algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, or wortmannin.

Drug Combination Comprising a Corticosteroid and (A) Serotonin Norepinephrine Reuptake Inhibitor or (B) a Noradrenaline Reuptake Inhibitor

In one embodiment, a drug combination that has anti-scarring activity comprises at least two agents wherein at least one agent is a corticosteroid and at least one second agent is selected from a serotonin norepinephrine reuptake inhibitor (SNRI) and a noradrenaline reuptake inhibitor (NARI) (or an analog or metabolite thereof). The drug combination may further include one or more additional compounds (e.g., a glucocorticoid receptor modulator, NSAID, COX-2 inhibitor, small molecule immunomodulator, DMARD, biologic, xanthine, anticholinergic compound, beta receptor agonist, bronchodilator, non-steroidal calcineurin inhibitor, vitamin D analog, psoralen, retinoid, or 5-amino salicylic acid). In a particular embodiment, the drug combination comprises a SNRI or a NARI (or an analog or metabolite thereof) and a glucocorticoid receptor modulator. In another embodiment, a drug combination is provided that includes an SNRI or NARI (or an analog or metabolite thereof) and a second compound selected from a xanthine, anticholinergic compound, beta receptor agonist, bronchodilator, non-steroidal calcineurin inhibitor, vitamin D analog, psoralen, retinoid, and 5-amino salicylic acid.

SNRIs that can be used in the drug combinations described herein include, without limitation, duloxetine, milnacipram, nefazodone, sibutramine, and venlafaxine. NARIs that can be included in the drug combinations described herein include, without limitation, atomoxetine, reboxetine, and MCI-225.

The corticosteroid and an SNRI or an NARI contained in the drug combination may be present in amounts that together are sufficient to treat or prevent an inflammatory response, disease, or disorder in a patient or subject in need thereof

Compounds useful in the drug combinations described herein include those described herein in any of their pharmaceutically acceptable forms, including isomers such as diastereomers and enantiomers, salts, esters, solvates, and polymorphs thereof, as well as racemic mixtures and pure isomers of the compounds described herein.

By “NARI” is meant any member of the class of compounds that (i) inhibit the uptake of norepinephrine by neurons of the central nervous system, (ii) have an inhibition constant (Ki) of 10 nM or less, and (iii) a ratio of Ki(norepinephrine) over Ki(serotonin)) of less than 0.01.

Corticosteroids and exemplary corticosteroid compounds are described in detail herein. By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin, which are described in detail herein. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

Serotonin Norepinephrine Reuptake Inhibitors

By “SNRI” is meant any member of the class of compounds that (i) inhibit the uptake of serotonin and norepinephrine by neurons of the central nervous system, (ii) have at least one inhibition constant (Ki) of 10 nM or less, and (iii) a ratio of Ki(norepinephrine) over Ki(serotonin)) of between 0.01 and 100, desirably between 0.1 and 10.

As described herein, a drug combination may comprise an SNRI, or a structural or functional analog thereof. Suitable SNRIs include duloxetine (Cymbalta™), milnacipram (Ixel™, Toledomin™), nefazodone (Serzone™), sibutramine (Meridia™, Reductil™), and venlafaxine (Effexor™, Efexor™, Trevilor™, Vandral™).

Duloxetine

Duloxetine has the following structure:

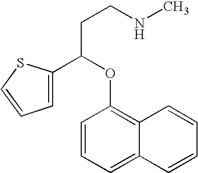

Structural analogs of duloxetine are those having the formula: 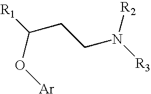
as well as pharmaceutically acceptable salts thereof, wherein R₁ is C₅-C₇ cycloalkyl, thienyl, halothienyl, (C₁-C₄alkyl) thienyl, furanyl, pyridyl, or thiazolyl; each of R₂ and R₃ Ar is, independently, hydrogen or methyl; Ar is 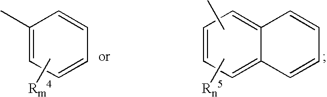
each R⁴ is, independently, halo, C₁-C₄ alkyl, C₁-C₃ alkoxy, or trifluoromethyl; each R⁵ is, independently, halo, C₁-C₄ alkyl, or trifluoromethyl; m is 0, 1, or 2; and n is 0 or 1.

Exemplary duloxetine structural analogs are N-methyl-3-(1-naphthalenyloxy)-3-(3-thienyl)propanamine phosphate; N-methyl-3-(2-naphthalenyloxy)-3-(cyclohexyl)propanamine citrate; N, N-dimethyl-3-(4-chloro-1-naphthalenyloxy)-3-(3-furanyl)propanamine hydrochloride; N-methyl-3-(5-methyl-2-naphthalenyloxy)-3-(2-thiazolyl)propanamine hydrobromide; N-methyl-3-[3-(trifluoromethyl)-1-naphthalenyloxy]-3-(3-methyl-2-thienyl)propanamine oxalate; N-methyl-3-(6-iodo-1-naphthalenyloxy)-3-(4pyridyl)propanamine maleate; N,N-dimethyl-3-(1-naphthalenyloxy)-3-(cycloheptyl)propanamine formate; N,N-dimethyl-3-(2-naphthalenyloxy)-3-(2-pyridyl)propanamine; N-methyl-3-(1-naphthalenyloxy)-3-(2-furanyl)propanamine sulfate; N-methyl-3-(4-methyl-1-naphthalenyloxy)-3-(4-thiazolyl)propanamine oxalate; N-methyl-3-(2-naphthalenyloxy)-3-(2-thienyl)propanamine hydrochloride; N,N-dimethyl-3-(6-iodo-2-naphthalenyloxy)-3-(4-bromo-3-thienyl)propanamine malonate; N,N-dimethyl-3-(1-naphthalenyloxy)-3-(3-pyridyl)propanamine hydroiodide; N,N-dimethyl-3-(4-methyl-2-naphthalenyloxy)-3-(3-furanyl)propanamine maleate; N-methyl-3-(2-naphthalenyloxy)-3-(cyclohexyl)propanamine caprate; N-methyl-3-(6-n-propyl-1-naphthalenyloxy)-3-(3-isopropyl-2-thienyl)propanamine citrate; N,N-dimethyl-3-(2-methyl-1-naphthalenyloxy)-3-(4-thiazolyl)propanamine monohydrogen phosphate; 3-(1-naphthalenyloxy)-3-(5-ethyl-3-thienyl)propanamine succinate; 3-[3-(trifluoromethyl)-1-naphthalenyloxy]-3-(pyridyl)propanamine acetate; N-methyl-3-(6-methyl-1-naphthalenyl-3-(4-chloro-2-thienyl)propanamine tartrate; 3-(2-naphthalenyloxy)-3-(cyclopentyl)propanamine; N-methyl-3-(4-n-butyl-1-naphthalenyloxy)-3-(3-furanyl)propanamine methanesulfonate; 3-(2-chloro-1-naphthalenyloxy)-3-(5-thiazolyl)propanamine oxalate; N-methyl-3-(1-naphthalenyloxy)-3-(3-furanyl)propanamine tartrate; N,N-dimethyl-3-(phenoxy)-3-(2-furanyl)propanamine oxalate; N,N-dimethyl-3-[4-(trifluoromethyl)phenoxy]-3-(cyclohexyl)propanamine hydrochloride; N-methyl-3-(4-methylphenoxy)-3-(4-chloro-2-thienyl)propanamine propionate; N-methyl-3-(phenoxy)-3-(3-pyridyl)propanamine oxalate; 3-2-chloro-4-(trifluoromethyl)phenoxy]-3-(2-thienyl)propanamine; N,N-dimethyl-3-(3-methoxyphenoxy)-3-(3-bromo-2-thienyl)propanamine citrate; N-methyl-3-(4-bromophenoxy)-3-(4-thiazolyl)propanamine maleate; N,N-dimethyl-3-(2-ethylphenoxy)-3-(5-methyl-3-thienyl)propanamine; N-methyl-3-(2-bromophenoxy)-3-(3-thienyl)propanamine succinate; N-methyl-3-(2,6-dimethylphenoxy)-3-(3-methyl-2-thienyl)propanamine acetate; 3-[3-(trifluoromethyl)phenoxy]-3-(3-furanyl)propanamine oxalate; N-methyl-3-(2,5-dichlorophenoxy)-3-(cyclopentyl)propanamine; 3-[4-(trifluoromethyl)phenoxy]-3-(2-thiazolyl)propanamine; N-methyl-3-(phenoxy)-3-(5-methyl-2-thienyl)propanamine citrate; 3-(4-methylphenoxy)-3-(4-pyridyl)propanamine hydrochloride; N,N-dimethyl-3-(3-methyl-5-bromophenoxy)-3-(3-thienyl)propanamine; N-methyl-3-(3-n-propylphenoxy)-3-(2-thienyl)propanamine hydrochloride; N-methyl-3-(phenoxy)-3-(3-thienyl)propanamine phosphate; N-methyl-3-(4-methoxyphenoxy)-3-(cycloheptyl)propanamine citrate; 3-(2-chlorophenoxy)-3-(5-thiazolyl)propanamine propionate; 3-2-chloro-4-(trifluoromethyl)phenoxy]-3-(3-thienyl)propanamine oxalate; 3-(phenoxy)-3-(4-methyl-2-thienyl)propanamine; N,N-dimethyl-3-(4-ethylphenoxy)-3-(3-pyridyl)propanamine maleate; and N,N-dimethyl-3-[4-(trifluoromethyl)phenoxy]-3-(2-pyridyl)propanamine. These compounds can be synthesized, for example, using the methods described in U.S. Pat. No. 4,956,388.

Milnacipram

Milnacipram has the following structure:

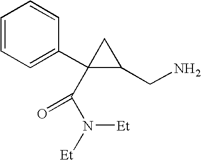

Structural analogs of milnacipram are those having the formula: 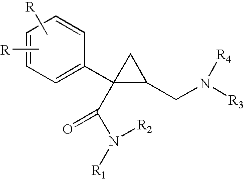
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Nefazodone

Nefazodone has the following structure:

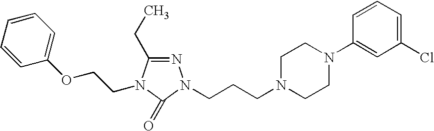

Structural analogs of nefazodone are those compounds having the formula: 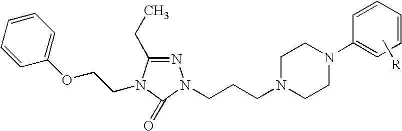
as well as pharmaceutically acceptable salts thereof, wherein R is halogen. Compounds having this formula can be synthesized, for example, using the methods described in U.S. Pat. No. 4,338,317.
Sibutramine

Sibutramine has the following structure:

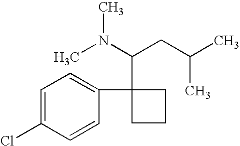

Structural analogs of sibutramine are those compounds having the formula: 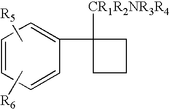
as well as pharmaceutically acceptable salts thereof, wherein R₁ is C₁₋₆ alkyl, C₂₋₆ alkenyl, C₂₋₆ alkynyl, C₃₋₇ cycloalkyl, cycloalkylalkyl, or optionally substituted phenyl (substituents include halogen and C₁₋₃ alkyl); R₂ is H or C₁₋₃ alkyl; each of R₃ and R₄ is, independently, H, formyl, or R₃ and R₄ together with the nitrogen atom form a heterocyclic ring system; each of R₅ and R₆ is, independently, H, halogen, CF₃, C₁₋₃ alkyl, C₁₋₃ alkoxy, C₁₋₃ alkylthio, or R₆ together with the carbon atoms to which they are attached form a second benzen ring.

Exemplary sibutramine structural analogs are 1-[1-(3,4-dichlorophenyl)cyclobutyl]ethylamine hydrochloride; N-methyl-1-[1-(3,4-dichlorophenyl)cyclobutyl]ethylamine hydrochloride; N,N-dimethyl-1-[1-(3,4-dichlorophenyl)cyclobutyl]ethylamine hydrochloride; 1-[1-(4-iodophenyl)cyclobutyl]ethylamine hydrochloride; N-methyl-1-[1-(4-iodophenyl)cyclobutyl]ethylamine hydrochloride; N,N-dimethyl-1-[1-(4-iodophenyl)cyclobutyl]ethylamine hydrochloride; N-methyl-1-[1-(2-naphthyl)cyclobutyl]ethylamine hydrochloride; N,N-dimethyl-1-[1-(4-chloro-3-trifluoromethylpheynl)cyclobutyl]ethylamine hydrochloride; 1-[1-(4-chlorophenyl)cyclobutyl]butylamine hydrochloride; N-methyl-1-[1-(4-chlorophenyl)cyclobutyl]butylamine hydrochloride; N,N-dimethyl-1-[1-(4-chlorophenyl)cyclobutyl]butyl amine hydrochloride; 1-[1-(3,4-dichlorophenyl)cyclobutyl]butylamine hydrochloride; N-methyl-1-[1-(3,4-dichlorophenyl)cyclobutyl]butylamine hydrochloride; N,N-dimethyl-1-[1-(3,4-dichlorophenyl)cyclobutyl]butylamine hydrochloride; 1-[1-(4-biphenylyl)cyclobutyl]butylamine hydrochloride; N,N-dimethyl-1-[1-(4-biphenylyl)cyclobutyl]butylamine hydrochloride; 1-[1-(4-chloro-3-fluorophenyl)cyclobutyl]butylamine hydrochloride; N-formyl-1-[1-(4-chloro-3-fluorophenyl)cyclobutyl]butylamine; 1-[1-(3-chloro-4-methylphenyl)cyclobutyl]butylamine hydrochloride; N-formyl-1-[1-phenylcyclobutyl]butylamine; 1-[1-(3-trifluoromethylphenyl)cyclobutyl]butylamine hydrochloride; 1-[1-(naphth-2-yl)cyclobutyl]butylamine hydrochloride; 1-[1-(6-chloronaphth-2-yl)cyclobutyl]butylamine; N-methyl-1-[1-(4-chlorophenyl)cyclobutyl]-2-methylpropylamine hydrochloride; 1-[1-(4-chlorophenyl)cyclobutyl]pentylamine hydrochloride; N-methyl-1-[1-(4-chlorophenyl)cyclobutyl]pentylamine hydrochloride; N,N-dimethyl-1-[1-phenylcyclobutyl]-3-methylbutylamine hydrochloride; 1-[1-(4-chlorophenyl)cyclobutyl-3-methylbutylamine hydrochloride; N-methyl-1-[1-(4-chlorophenyl)cyclobutyl]-3-methylbutylamine hydrochloride; N,N-dimethyl-1-[1-(4-chlorophenyl)cyclobutyl]-3-methylbutylamine hydrochloride; N-formyl-1-[1-(4-chlorophenyl)cyclobutyl]-3-methylbutylamine; N,N-dimethyl-1-[1-(3,4-dichlorophenyl)cyclobutyl]-3-methylbutylamine hydrochloride; N-methyl-1-[1-(naphth-2-yl)cyclobutyl]-3-methylbutylamine hydrochloride; N-methyl-1-[1-(3,4-dimethylphenyl)cyclobutyl]-3-methylbutylamine hydrochloride; [1-(4-chlorophenyl)cyclobutyl](cyclopropyl)methylamine hydrochloride; N-methyl-[1-(4-chlorophenyl)cyclobutyl](cyclopentyl)methylamine hydrochloride; [1-(4-chlorophenyl)cyclobutyl](cyclohexyl)methylamine hydrochloride; N-methyl-[1-(4-chlorophenyl)cyclobutyl](cyclohexyl)methylamine hydrochloride; [1-(3,4-dichlorophenyl)cyclobutyl](cyclohexyl)methylamine hydrochloride; N-methyl-[1-(3,4-dichlorophenyl)cyclobutyl](cyclohexyl)methylamine hydrochloride; [1-(4-chlorophenyl)cyclobutyl](cycloheptyl)methylamine hydrochloride; 1-[1-(4-chlorophenyl)cyclobutyl]-2-cyclopropylethylamine hydrochloride; N,N-dimethyl-1-[1-(4-chlorophenyl)cyclobutyl]-2-cyclohexylethylamine hydrochloride; α-[1-(4-chlorophenyl)cyclobutyl]benzylamine hydrochloride; N-methyl-α-[1-(4-chlorophenyl)cyclobutyl]benzylamine hydrochloride; 1-[1-(4-chloro-2-fluorophenyl)cyclobutyl]butylamine; N,N-dimethyl-1-[1-(4-chloro-2-fluorophenyl)cyclobutyl]butylamine hydrochloride; N-ethyl-1-[1-(3,4-dichlorophenyl)cyclobutyl]ethylamine hydrochloride; and N,N-diethyl-1-[1-(3,4-dichlorophenyl)cyclobutyl]ethylamine hydrochloride. These compounds can be synthesized, for example, using the methods described in U.S. Pat. No. 4,814,352.

Venlafaxine

Venlafaxine has the following structure:

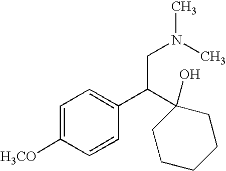

Structural analogs of venlafaxine are those compounds having the formula: 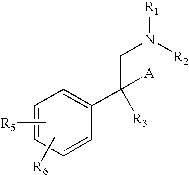
as well as pharmaceutically acceptable salts thereof, wherein A is a moiety of the formula: 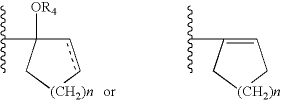
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.
Noradrenaline Reuptake Inhibitors

The drug combinations described herein may comprise an NARI, or a structural or functional analog thereof. Suitable NARI compounds include atomoxetine (Strattera™), reboxetine (Edronax™), and MCI-225.

Atomoxetine

Atomoxetine has the following structure:

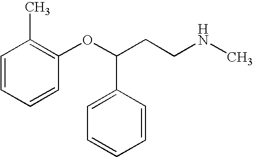

Structural analogs of atomoxetine are those having the formula: 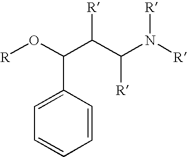
as well as pharmaceutically acceptable salts thereof, wherein each R′ is, independently, hydrogen or methyl; and R is napthyl or 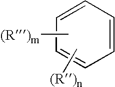
wherein each of R″ and R″′ is, independently, halo, trifluoromethyl, C₁₋₄ alkyl, C₁₋₃ alkoxy, or C₃₋₄ alkenyl; and each of n and m is, independently, 0, 1, or 2.

Exemplary atomoxetine structural analogs are 3-(p-isopropoxyphenoxy)-3-phenylpropylamine methanesulfonate; N,N-dimethyl 3-(3′,4′-dimethoxyphenoxy)-3-phenylpropylamine p-hydroxybenzoate; N,N-dimethyl 3-(α-naphthoxy)-3-phenylpropylamine bromide; N,N-dimethyl 3-(.beta.-naphthoxy)-3-phenyl-1-methylpropylamine iodide; 3-(2′-methyl-4′,5′-dichlorophenoxy)-3-phenylpropylamine nitrate; 3-(p-t-butylphenoxy)-3-phenylpropylamine glutarate; N-methyl 3-(2′-chloro-p-tolyloxy)-3-phenyl-1-methylpropylamine lactate; 3-(2′,4′-dichlorophenoxy)-3-phenyl-2-methylpropylamine citrate; N,N-dimethyl 3-(m-anisyloxy)-3-phenyl-1-methylpropylamine maleate; N-methyl 3-(p-tolyloxy)-3-phenylpropylamine sulfate; N,N-dimethyl 3-(2′,4′-difluorophenoxy)-3-phenylpropylamine 2,4-dinitrobenzoate; 3-(o-ethylphenoxy)-3-phenylpropylamine dihydrogen phosphate; N-methyl 3-(2′-chloro-4′-isopropylphenoxy)-3-phenyl-2-methylpropylamine maleate; N,N-dimethyl 3-(2′-alkyl-4′-fluorophenoxy)-3-phenyl-propylamine succinate; N,N-dimethyl 3-(o-isopropoxyphenoxy)-3-phenyl-propylamine phenylacetate; N,N-dimethyl 3-(o-bromophenoxy)-3-phenyl-propylamine .beta.-phenylpropionate; N-methyl 3-(p-iodophenoxy)-3-phenyl-propylamine propiolate; and N-methyl 3-(3-n-propylphenoxy)-3-phenyl-propylamine decanoate. These compounds can be synthesized, for example, using the methods described in U.S. Pat. No. 4,314,081.

Reboxetine

Reboxetine has the following structure:

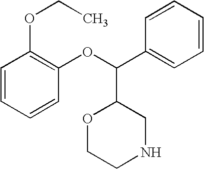

Structural analogs of reboxetine are those having the formula: 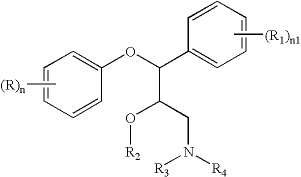
as well as pharmaceutically acceptable salts thereof, wherein each of n and n1 is, independently, 1, 2, or 3; each of R and R₁ is, independently, hydrogen, halogen, halo-C₁₋₆ alkyl, hydroxy, C₁₋₆ alkyl optionally substituted, C₁-C₆ alkoxy, aryl-C₁₋₆ alkoxy optionally substituted, NO₂, NR₅R₆, wherein each of R₅ and R₆ is, independently, hydrogen, C₁₋₆ alkyl, or two adjacent R groups or two adjacent R₁ groups, taken together, form the —O—CH₂—O— radical; R₂ is hydrogen; C₁₋₁₂ alkyl optionally substituted, or aryl-C₁₋₆ alkyl; each of R₃ and R₄ is, independently, hydrogen, C₁₋₆ alkyl optionally substituted, C₂₋₄ alkenyl,C₂₋₄ alkynyl, aryl-C₁₋₄ alkyl optionally substituted, C₃₋₇ cycloalkyl optionally substituted, or R₃ and R₄ with the nitrogen atom to which they are bounded form a pentatomic or hexatomic saturated or unsaturated, optionally substituted, heteromonocyclic radical optionally containing other heteroatoms belonging to the class of O,S and N; or R₂ and R₄, taken together, form the —CH₂CH₂— radical.

Exemplary reboxetine structural analogs are 2-(α-phenoxy-benzyl)-morpholine; 2-[α-(2-methoxy-phenoxy)-benzyl]-morpholine; 2-[α-(3-methoxy-phenoxy)-benzyl]-morpholine; 2-[α-(4-methoxy-phenoxy)-benzyl]-morpholine; 2-[α-(2-ethoxy-phenoxy)-benzyl]-morpholine; 2-[α-(4-chloro-phenoxy)-benzyl]-morpholine; 2-[α-(3,4-methylendioxy-phenoxy)-benzyl]-morpholine; 2-[α-(2-methoxy-phenoxy)-2-methoxy-benzyl]-morpholine; 2-[α-(2-ethoxy-phenoxy)-2-methoxy-benzyl]-morpholine; 2-[α-(2-ethoxy-phenoxy)-4-ethoxy-benzyl]-morpholine; 2-[α-(4-chloro-phenoxy)-4-ethoxy-benzyl]-morpholine; 2-[α-(2-methoxy-phenoxy)-4-ethoxy-benzyl]-morpholine; 2-[α-(2-methoxy-phenoxy)-2-chloro-benzyl]-morpholine; 2-[α-(2-ethoxy-phenoxy)-2-chloro-benzyl]-morpholine; 2-[α-(2-methoxy-phenoxy)-3-chloro-benzyl]-morpholine; 2-[α-(2-ethoxy-phenoxy)-3-chloro-benzyl]-morpholine; 2-[α-(2-ethoxy-phenoxy)-4-chloro-benzyl]-morpholine; 2-[α-(2-methoxy-phenoxy)-4-chloro-benzyl]-morpholine; 2-[α-(2-methoxy-phenoxy)-4-trifluoromethyl-benzyl]-morpholine; 2-[α-(4-ethoxy-phenoxy)-4-trifluoromethyl-benzyl]-morpholine; 2-[α-(2-methoxy-phenoxy)-3,4-dichloro-benzyl]-morpholine; 2-[α-(2-ethoxy-phenoxy)-3,4-dichloro-benzyl]-morpholine; 4-methyl-2-[α-(2-methoxy-phenoxy)-benzyl]-morpholine; 4-methyl-2-[α-(2-ethoxy-phenoxy)-benzyl]-morpholine; 4-methyl-2-[α-(2-methoxy-phenoxy)-3-chloro-benzyl]-morpholine; 4-methyl-2-[α-(2-ethoxy-phenoxy)-3-chloro-benzyl]-morpholine; 4-methyl-2-[α-(2-ethoxy-phenoxy)-4-chloro-benzyl]-morpholine; 4-methyl-2-[α-(2-methoxy-phenoxy)-4-chloro-benzyl]-morpholine; 4-methyl-2-[α-(2-methoxy-phenoxy)-4-trifluoromethyl-benzyl]-morpholin e; 4-methyl-2-[α-(2-ethoxy-phenoxy)-4-trifluoromethyl-benzyl]-morpholine; 4-isopropyl-2-[α-(2-methoxy-phenoxy)-benzyl]-morpholine; 4-isopropyl-2-[α-(2-ethoxy-phenoxy)-benzyl]-morpholine; 4-isopropyl-2-[α-(2-methoxy-phenoxy)-3-chloro-benzyl]-morpholine; 4-isopropyl-2-[α-(2-ethoxy-phenoxy)-3-chloro-benzyl]-morpholine; 4-isopropyl-2-[α-(2-ethoxy-phenoxy)-4-chloro-benzyl]-morpholine; 4-isopropyl-2-[α-(2-methoxy-phenoxy)-4-chloro-benzyl]-morpholine; 4-isopropyl-2-[α-(2-methoxy-phenoxy)-4-trifluoromethyl-benzyl]-morpholine; 4-isopropyl-2-[α-(2-ethoxy-phenoxy)-4-trifluoromethyl-benzyl]-morpholine; N-methyl-2-hydroxy-3-phenoxy-3-phenyl-propylamine; N-methyl-2-hydroxy-3-(2-methoxy-phenoxy)-3-phenyl-propylamine; N-methyl-2-hydroxy-3-(2-ethoxy-phenoxy)-3-phenyl-propylamine; N-methyl-2-hydroxy-3-(4-chloro-phenoxy)-3-phenyl-propylamine; N-methyl-2-hydroxy-3-(3,4-methylendioxy-phenoxy)-3-phenyl-propylamine; N-methyl-2-hydroxy-3-(2-methoxy-phenoxy)-3-(2-chloro-phenyl)-propylamine; N-methyl-2-hydroxy-3-(2-ethoxy-phenoxy)-3-(2-chloro-phenyl)-propylamine; N-methyl-2-hydroxy-3-(2-methoxy-phenoxy)-3-(3-chloro-phenyl)-propylamine; N-methyl-2-hydroxy-3-(2-ethoxy-phenoxy)-3-(3-chloro-phenyl)-propylamine; N-methyl-2-hydroxy-3-(2-methoxy-phenoxy)-3-(4-chloro-phenyl)-propylamine; N-methyl-2-hydroxy-3-(2-ethoxy-phenoxy)-3-(4-chloro-phenyl)-propylamine; N-methyl-2-hydroxy-3-(2-methoxy-phenoxy)-3-(4-trifluoromethyl-phenyl)-propylamine; N-methyl-2-hydroxy-3-(2-ethoxy-phenoxy)-3-(4-trifluoromethyl-phenyl)-propyl amine; N-methyl-2-hydroxy-3-(2-methoxy-phenoxy)-3-(3,4-dichloro-phenyl)-propylamine; N-methyl-2-hydroxy-3-(2-ethoxy-phenoxy)-3-(3,4-dichloro-phenyl)-propylamine; N-methyl-2-methoxy-3-phenoxy-3-phenyl-propylamine; N-methyl-2-methoxy-3-(2-methoxy-phenoxy)-3-phenyl-propylamine; N-methyl-2-methoxy-3-(2-ethoxy-phenoxy)-3-phenyl-propylamine; N-methyl-2-methoxy-3-(4-chloro-phenoxy)-3-phenyl-propylamine; N-methyl-2-methoxy-3-(3,4-methylenedioxy-phenoxy)-3-phenyl-propylamine; N-methyl-2-methoxy-3-phenoxy-3-(2-chloro-phenyl)-propylamine; N-methyl-2-methoxy-3-(2-methoxy-phenoxy)-3-(2-chloro-phenyl)-propylamine; N-methyl-2-methoxy-3-(2-ethoxy-phenoxy)-3-(2-chloro-phenyl)-propylamine; N-methyl-2-methoxy-3-(2-methoxy-phenoxy)-3-(3-chloro-phenyl)-propylamine; N-methyl-2-methoxy-3-(2-ethoxy-phenoxy)-3-(3-chloro-phenyl)-propylamine; N-methyl-2-methoxy-3-(2-methoxy-phenoxy)-3-(4-chloro-phenyl)-propylamine; N-methyl-2-methoxy-3-(2-ethoxy-phenoxy)-3-(4-chloro-phenyl)-propylamine; N-methyl-2-methoxy-3-(2-methoxy-phenoxy)-3-(4-trifluoromethyl-phenyl)-propylamine; N-methyl-2-methoxy-3-(2-ethoxy-phenoxy)-3-(4-trifluoromethyl-phenyl)-propylamine; N-methyl-2-methoxy-3-(2-methoxy-phenoxy)-3-(3,4-dichloro-phenyl)-propylamine; and N-methyl-2-methoxy-3-(2-ethoxy-phenoxy)-3-(3,4-dichloro-phenyl)-propylamine. These compounds can be synthesized, for example, using the methods described in U.S. Pat. No. 4,229,449.

MCI-225

MCI-225 (4-(2-fluorophenyl)-6-methyl-2-piperazinothieno [2,3-d]pyrimidine) has the following structure:

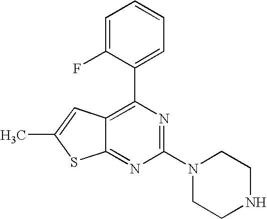

Structural analogs of MCI-225 are those having the formula: 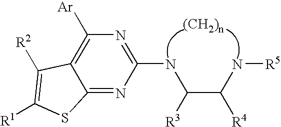
as well as pharmaceutically acceptable salts thereof, wherein each of R¹ and R² is, independently, hydrogen, halogen, C₁-C₆ alkyl, or R¹ and R² form a 5 to 6-membered cycloalkylene ring together with two carbon atoms of thienyl group; each of R³ and R⁴ is, independently, hydrogen or C₁-C₆ alkyl; R⁵ is hydrogen, C₁-C₆ alkyl, 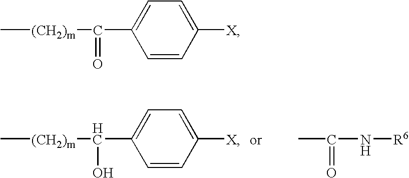
in which m is an integer of 1-3, X is a halogen, and R⁶ is C₁-C₆ alkyl; Ar is phenyl, 2-thienyl, or 3-thienyl, each of which may substituted by halogen, C₁-C₆ alkyl, C₁-C₆ alkoxy (e.g., methoxy, ethoxy, propoxy, and butoxy), hydroxyl, nitro, amino, cyano, or alkyl-substituted amino (e.g., methylamino, ethylamino, dimethylamino, and diethylamino); and n is 2 or 3.

Exemplary MCI-225 structural analogs are 6-methyl-4-phenyl-2-piperazinyl-thieno[2,3-d]pyrimidine; 5,6-dimethyl-4-phenyl-2-piperazinyl-thieno[2,3-d]pyrimidine; 5-methyl-4-phenyl-2-piperazinyl-thieno[2,3-d]pyrimidine; 6-chloro-4-phenyl-2-piperazinyl-thieno[2,3-d]pyrimidine; 4-(2-bromophenyl)-6-methyl-2-piperazinyl-thieno[2,3-d]pyrimidine; 6-methyl-4-(2-methylphenyl)-2-piperazinyl-thieno[2,3-d]pyrimidine; and 4-(2-cyanophenyl)-6-methyl-2-piperazinyl-thieno[2,3-d]. These compounds can be synthesized, for example, using the methods described in U.S. Pat. No. 4,695,568.

In still other embodiments, certain other compounds can be used in drug combinations described herein instead of an SNRI or NARI and include 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554 (Piperazine, 1-(3-(1,3-benzodioxol-5-yloxy)propyl)-4-phenyl); CP 53261(N-desmethylsertraline); O-desmethylvenlafaxine; WY 45,818 (1-(2-(dimethylamino)-1-(2-chlorophenyl)ethyl)cyclohexanol); WY 45,881 (1-(1-(3,4-dichlorophenyl)-2-(dimethylamino)ethyl)cyclohexanol); N-(3-fluoropropyl)paroxetine; and Lu 19005 (3-(3,4-dichlorophenyl)-N-methyl-1-indanamine hydrochloride).

Compounds useful for the drug combinations described herein include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “paroxetine” is meant the free base, as well as any pharmaceutically acceptable salt thereof (e.g., paroxetine maleate, paroxetine hydrochloride hemihydrate, and paroxetine mesylate).

Corticosteroids

In one embodiment, one or more corticosteroid may be combined or formulated with an SNRI or NARI, or analog or metabolite thereof, in a drug combination. Suitable corticosteroids include any one of the corticosteroid compounds described herein or known in the art.

Steroid Receptor Modulators

Steroid receptor modulators (e.g., antagonists and agonists) may be used as a substitute for or in addition to a corticosteroid in the drug combination. Thus, in one embodiment, the drug combination features the combination of an SNRI or NARI (or analog or metabolite thereof) and a glucocorticoid receptor modulator or other steroid receptor modulator.

Glucocorticoid receptor modulators that may used in the drug combinations described herein include compounds described in U.S. Pat. Nos. 6,380,207, 6,380,223, 6,448,405, 6,506,766, and 6,570,020, U.S. Patent Application Publication Nos. 20030176478, 20030171585, 20030120081, 20030073703,2002015631, 20020147336, 20020107235, 20020103217, and 20010041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the drug combinations described herein A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

In one embodiment, as a substitute for or in addition to a corticosteroid in the drug combinations described herein, one or more agents that also act as bronchodilators may be included in the combination, including xanthines (e.g., theophylline), anticholinergic compounds (e.g., ipratropium, tiotropium), biologics, small molecule immunomodulators, and beta receptor agonists/bronchdilators (e.g., lbuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, the drug combination comprises an SNRI or NARI (or analog or metabolite thereof) and/or a corticosteroid and/or one or more of the aforementioned agents.

In another embodiment, as a substitute for or in addition to a corticosteroid in the drug combinations described herein, one or more agents that also acts as antipsoriatic agents may be included in the drug combination. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the drug combination features the combination of an SNRI or NARI (or analog or metabolite thereof) and/or a corticosteroid and/or one or more of the aforementioned agents.

In another embodiment, as a substitute for or in addition to a corticosteroid in the drug combinations described herein, one or more agents typically used to treat inflammatory bowel disease may be included in the drug combination. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the drug combinations described herein feature the combination of an SNRI or NARI (or analog or metabolite thereof) and/or a corticosteroid and/or one or more of any of the foregoing agents.

In still another embodiment, one or more agents typically used to treat rheumatoid arthritis may be used as a substitute for or in addition to a corticosteroid in the drug combinations described herein. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the drug combination features the combination of an SNRI or NARI (or analog or metabolite thereof) and/or a corticosteroid and/or one or more of any of the foregoing agents.

In yet another embodiment, one or more agents typically used to treat asthma may be used as a substitute for or in addition to a corticosteroid in the drug combinations described herein. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, a drug combination features the combination of an SNRI or NARI (or analog or metabolite thereof) and/or a corticosteroid and/or one or more of any of the foregoing agents.

Also provided herein are drug combinations employing an SNRI or NARI and a non-steroidal immunophilin-dependent immunosuppressant (NsIDI), optionally with a corticosteroid or other agent described herein.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

Cyclosporines

The cyclosporines are examples of calcineurin inhibitors and are fungal metabolites that comprise a class of cyclic oligopeptides that act as immunosuppressants. As described herein, Cyclosporine A, and its deuterated analogue ISAtx247, is a hydrophobic cyclic polypeptide consisting of eleven amino acids. Cyclosporine A binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T-cell-dependent immune response by inhibiting antigen-triggered signal transduction. This inhibition decreases the expression of proinflammatory cytokines, such as IL-2.

Many cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala (3-acetylamino)-8-Cs, Thr-2-Cs, and D-MeSer-3-Cs, D-Ser (O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

To counteract the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL™).

Tacrolimus

As described herein, tacrolimus (PROGRAF, Fujisawa), also known as FK506, is an immunosuppressive agent that targets T-cell intracellular signal transduction pathways. Tacrolimus binds to an intracellular protein FK506 binding protein (FKBP-12) that is not structurally related to cyclophilin (Harding et al., Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of NFAT, a nuclear component that initiates gene transcription required for lymphokine (e.g., IL-2, gamma interferon) production and T-cell activation. Thus, tacrolimus inhibits T-cell activation.

Tacrolimus is a macrolide antibiotic that is produced by Streptomyces tsukubaensis. Tacrolimus suppresses the immune system and prolongs the survival of transplanted organs. Tacrolimus is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 9% sodium chloride or 5% dextrose prior to injection.

Tacrolimus and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides ate described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus and Ascomycin Derivatives

Ascomycin is a close structural analog of FK506 and is a potent immunosuppressant. It binds to FKBP-12 and suppresses its proline rotamase activity. The ascomycin-FKBP complex inhibits calcineurin, a type 2B phosphatase.

Pimecrolimus (also known as SDZ ASM-981) is a 33-epi-chloro derivative of the ascomycin. It is produced by the strain Streptomyces hygroscopicus var. ascomyceitus. Like tacrolimus, pimecrolimus (ELIDEL™, Novartis) binds FKBP-12, inhibits calcineurin phosphatase activity, and inhibits T-cell activation by blocking the transcription of early cytokines. In particular, pimecrolimus inhibits IL-2 production and the release of other proinflammatory cytokines.

Pimecrolimus structural and functional analogs are described in U.S. Pat. No. 6,384,073. Pimecrolimus is used for the treatment of atopic dermatitis. Pimecrolimus is currently available as a 1% cream.

Rapamycin

Rapamycin (Rapamune™ sirolimus, Wyeth) is a cyclic lactone produced by Steptomyces hygroscopicus. Rapamycin is an immunosuppressive agent that inhibits T-lymphocyte activation and proliferation. Like cyclosporines, tacrolimus, and pimecrolimus, rapamycin forms a complex with the immunophilin FKBP-12, but the rapamycin-FKBP-12 complex does not inhibit calcineurin phosphatase activity. The rapamycin-immunophilin complex binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Everolimus (40-O-(2-hydroxyethyl)rapamycin; CERTICAN™; Novartis) is an immunosuppressive macrolide that is structurally related to rapamycin, and has been found to be particularly effective at preventing acute rejection of organ transplant when give in combination with cyclosporin A. By way of background, and as described herein, rapamycin is currently available for oral administration in liquid and tablet formulations.

Peptide Moieties

Peptides, peptide mimetics, peptide fragments, either natural, synthetic or chemically modified, that impair the calcineurin-mediated dephosphorylation and nuclear translocation of NFAT are suitable for inclusion in the drug combinations described herein. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcinuerin inhibitors, these agents are useful in the drug combinations described herein.

In other embodiments, a drug combination may further comprise other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies may be useful for the treatment of or prevention of an inflammatory response or autoimmune response in combination with other anti-cytokine agents or in combination with agents that modulate the immune response, such as agents that influence cell adhesion, or biologics (i.e., agents that block the action of IL-6, IL-1, IL-2, IL-12, IL-15 or TNFα (e.g., etanercept, adelimumab, infliximab, or CDP-870). For example (that of agents blocking the effect of TNFα), when the combination therapy reduces the production of cytokines, etanercept or infliximab may affect the remaining fraction of inflammatory cytokines.

In certain particular embodiments, a drug combination is provided that comprises a serotonin norepinephrine reuptake inhibitor (SNRI) or noradrenaline reuptake inhibitor (NARI) or analog thereof and a corticosteroid. In a particular embodiment, the SNRI is duloxetine, milnacipram, nefazodone, sibutramine, or venlafaxine, and in another particular embodiment, the NARI is atomoxetine, reboxetine, or MCI-225. In a specific embodiment, the corticosteroid is prednisolone, cortisone, budesonide, dexamethasone, hydrocortisone, methylprednisolone, fluticasone, prednisone, triamcinolone, or diflorasone. In a more specific embodiment, the SNRI is duloxetine or venlafaxine and the corticosteroid is prednisolone. In another specific embodiment, the NARI is atomoxetine or MCI-225 and the corticosteroid is prednisolone.

In another embodiment, the drug combination may further comprise an NSAID, COX-2 inhibitor, biologic, small molecule immunomodulator, DMARD, xanthine, anticholinergic compound, beta receptor agonist, bronchodilator, non-steroidal calcineurin inhibitor, vitamin D analog, psoralen, retinoid, or 5-amino salicylic acid. In particular embodiments, the NSAID is ibuprofen, diclofenac, or naproxen, and in other particular embodiments, the COX-2 inhibitor is rofecoxib, celecoxib, valdecoxib, or lumiracoxib. In other particular embodiments, the biologic is adelimumab, etanercept, or infliximab, and in other particular embodiments, the DMARD is methotrexate or leflunomide. In one particular embodiment, the xanthine is theophylline. In another embodiment, the anticholinergic compound is ipratropium or tiotropium; in other particular embodiments, the beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline. In still other particular embodiments, the non-steroidal calcineurin inhibitor is cyclosporine, tacrolimus, pimecrolimus, or ISAtx247, and in other more particular embodiments, vitamin D analog is calcipotriene or calcipotriol. In another particular embodiment, psoralen is methoxsalen. In another embodiment, the retinoid is acitretin or tazoretene, and in another embodiment, 5-amino salicylic acid is mesalamine, sulfasalazine, balsalazide disodium, or olsalazine sodium. In an additional embodiment, a small molecule immunomodulator is VX 702, SCIO 469, doramapimod, RO 30201195, SCIO 323, DPC 333, pranalcasan, mycophenolate, or merimepodib.

Drug Combination Comprising a Non-Steroidal Immunophilin-Dependent Immunosuppressant (NsIDI) and a Non-Steroidal Immunophilin-Dependent Immunosuppressant Enhancer (NsIDIE)

In one embodiment, a drug combination that has anti-scarring activity comprises at least two agents wherein at least one agent is a non-steroidal immunophilin-dependent immunosuppressant (NsIDI) (e.g., cyclosporine A) and at least one second agent is a non-steroidal immunophilin-dependent immunosuppressant enhancer (NsIDIE) (e.g., a selective serotonin reuptake inhibitor (SSRI), a tricyclic antidepressant, a phenoxy phenol, an antihistamine, a phenothiazine, or a mu opioid receptor agonist). In certain embodiments, the drug combination may further comprise a non-steroidal anti-inflammatory drug (NSAID), a COX-2 inhibitor, a biologic, a disease-modifying anti-rheumatic drugs (DMARD), a xanthine, an anticholinergic compound, a beta receptor agonist, a bronchodilator, a non-steroidal calcineurin inhibitor, a vitamin D analog, a psoralen, a retinoid, or a 5-amino salicylic acid.

In certain embodiments described herein, an NsIDI is, for example, a calcineurin inhibitor, such as cyclosporine, tacrolimus, ascomycin, pimecrolimus, or ISAtx247, or an FK506-binding protein, such as rapamycin or everolimus. In other embodiments, an NsIDI enhancer (NsIDIE) is, for example, a selective serotonin reuptake inhibitor (SSRI), a tricyclic antidepressant (TCA), a phenoxy phenol, an antihistamine, a phenothiazine, or a mu opioid receptor agonist.

By “non-steroidal immunophilin-dependent immunosuppressant enhancer” or “NsIDIE” is meant any compound that increases the efficacy of a non-steroidal immunophilin-dependent immunosuppressant. NsIDIEs include selective serotonin reuptake inhibitors, tricyclic antidepressants, phenoxy phenols (e.g., triclosan), antihistamines, phenothiazines, and mu opioid receptor agonists.

By “antihistamine” is meant a compound that blocks the action of histamine. Classes of antihistamines include, but are not limited to, ethanolamines, ethylenediamine, phenothiazine, alkylamines, piperazines, and piperidines.

By “selective serotonin reuptake inhibitor” or “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are described herein.

Compounds useful for the drug combinations described herein include those described herein in any of their pharmaceutically acceptable forms, including isomers such as diastereomers and enantiomers, salts, esters, solvates, and polymorphs thereof, as well as racemic mixtures and pure isomers of the compounds described herein.

A tricyclic compound, which includes a “tricyclic antidepressant” or “TCA” compound includes a compound having one of the formulas (I), (II), (III), or (IV), which are described in greater detail herein. Exemplary tricyclic antidepressants are also provided herein and include maprotiline, amoxapine, 8-hydroxyamoxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, and protriptyline.

By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated. Corticosteroids are described in detail herein and examples of corticosteroids are also provided herein.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

In the generic descriptions of compounds of this invention, such as for example, with respect to the structures having any one of formula (I), (II), (III), or (IV), the number of atoms of a particular type in a substituent group is generally given as a range, e.g., an alkyl group containing from 1 to 7 carbon atoms or C1-7 alkyl. Reference to such a range is intended to include specific references to groups having each of the integer number of atoms within the specified range. For example, an alkyl group from 1 to 7 carbon atoms includes each of C1, C2, C3, C4, C5, C6, and C7. A C1-7 heteroalkyl, for example, includes from 1 to 7 carbon atoms in addition to one or more heteroatoms. Other numbers of atoms and other types of atoms may be indicated in a similar manner.

Compounds include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “paroxetine” is meant the free base, as well as any pharmaceutically acceptable salt thereof (e.g., paroxetine maleate, paroxetine hydrochloride hemihydrate, and paroxetine mesylate).

Provided herein are drug combinations that comprise an effective amount of a non-steroidal immunophilin-dependent immunosuppressant (NsIDI), such as cyclosporine, and a non-steroidal immunophilin-dependent immunosuppressant enhancer (NSIDIE), e.g., a selective serotonin reuptake inhibitor, a tricyclic antidepressant, a phenoxy phenol, an antihistamine, a phenothiazine, or a mu opioid receptor agonist. The combinations are described in greater detail below.

Non-Steroidal Immunophilin-Dependent Immunosuppressants

In one embodiment, the drug combination comprises an NsIDI and an NsIDIE, optionally with a corticosteroid or other agent described herein. By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders. The cyclosporines, tacrolimus, ascomycin, pimecrolimus, rapamycin, and peptide moities are described in detail above.

Selective Serotonin Reuptake Inhibitors

In one embodiment, the drug combination comprises a selective serotonin reuptake inhibitor (SSRI), or a structural or functional analog thereof in combination with a non-steroidal immunophilin-dependent immunosuppressant (NsIDI). Suitable SSRIs include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipram (e.g., minlacipran hydrochloride); paroxetine (e.g., paroxetine hydrochloride hemihydrate; paroxetine maleate; paroxetine mesylate); sertraline (e.g., sertraline hydrochloride); sibutramine, tametraline hydrochloride; viqualine; and zimeldine (e.g., zimeldine hydrochloride).

SSRIs are drugs that inhibit 5-hydroxytryptamine (5-HT) uptake by neurons of the central nervous system. SSRIs show selectivity with respect to 5-HT over norepinephrine uptake. They are less likely than tricyclic antidepressants to cause anticholinergic side effects and are less dangerous in overdose. SSRIs, such as paroxetine, sertraline, fluoxetine, citalopram, fluvoxamine, nor₁-citalopram, venlafaxine, milnacipram, nor₂-citalopram, nor-fluoxetine, or nor-sertraline are used to treat a variety of psychiatric disorders, including depression, anxiety disorders, panic attacks, and obsessive-compulsive disorder. Dosages given here are the standard recommended doses for psychiatric disorders. In practicing the methods of the invention, effective amounts may be different.

Cericlamine

Cericlamine has the following structure:

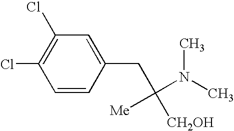

Structural analogs of cericlamine are those having the formula: 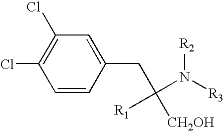
as well as pharmaceutically acceptable salts thereof, wherein R₁ is a C₁-C₄ alkyl and R₂ is H or C₁₋₄ alkyl, R₃ is H, C₁₋₄ alkyl, C₂₋₄ alkenyl, phenylalkyl or cycloalkylalkyl with 3 to 6 cyclic carbon atoms, alkanoyl, phenylalkanoyl or cycloalkylcarbonyl having 3 to 6 cyclic carbon atoms, or R₂ and R₃ form, together with the nitrogen atom to which they are linked, a heterocycle saturated with 5 to 7 chain links which can have, as the second heteroatom not directly connected to the nitrogen atom, an oxygen, a sulphur or a nitrogen, the latter nitrogen heteroatom possibly carrying a C₂₋₄ alkyl.

Exemplary cericlamine structural analogs are 2-methyl-2-amino-3-(3,4-dichlorophenyl)-propanol, 2-pentyl-2-amino-3-(3,4-dichlorophenyl)-propanol, 2-methyl-2-methylamino-3-(3,4-dichlorophenyl)-propanol, 2-methyl-2-dimethylamino-3-(3,4-dichlorophenyl)-propanol, and pharmaceutically acceptable salts of any thereof.

Citalopram

Citalopram HBr (CELEXA™) is a racemic bicyclic phthalane derivative designated (±)-1-(3-dimethylaminopropyl)-1-(4-fluorophenyl)-1,3-dihydroisobenzofuran-5-carbonitrile, HBr. Citalopram undergoes extensive metabolization; nor₁-citalopram and nor₂-citalopram are the main metabolites. By way of background, Citalopram is available in 10 mg, 20 mg, and 40 mg tablets for oral administration. CELEXA™ oral solution contains citalopram HBr equivalent to 2 mg/mL citalopram base. CELEXA™ is typically administered at an initial dose of 20 mg once daily, generally with an increase to a dose of 40 mg/day. Dose increases typically occur in increments of 20 mg at intervals of no less than one week.

Citalopram has the following structure:

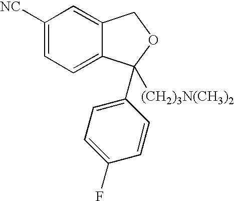

Structural analogs of citalopram are those having the formula: 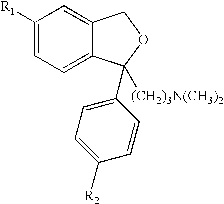
as well as pharmaceutically acceptable salts thereof, wherein each of R1 and R2 is independently selected from the group consisting of bromo, chloro, fluoro, trifluoromethyl, cyano and R—CO—, wherein R is C1-4 alkyl.

Exemplary citalopram structural analogs (which are thus SSRI structural analogs) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Closvoxamine has the following structure:

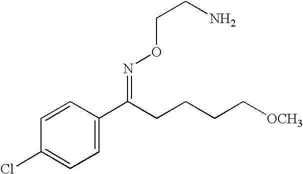

Structural analogs of clovoxamine are those having the formula: 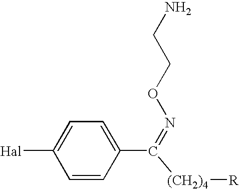
as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

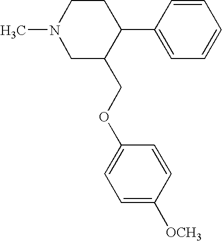

Structural analogs of femoxetine are those having the formula: 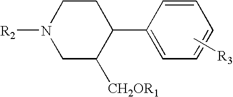
wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine hydrochloride((±)-N-methyl-3-phenyl-3-[((alpha),(alpha),(alpha)-trifluoro-p-tolyl)oxy]propylamine hydrochloride) is sold as PROZAC™ in 10 mg, 20 mg, and 40 mg tablets for oral administration. The main metabolite of fluoxetine is nor-fluoxetine.

Fluoxetine has the following structure:

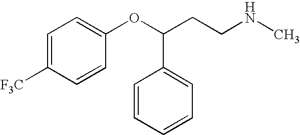

Structural analogs of fluoxetine are those compounds having the formula: 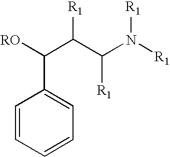
as well as pharmaceutically acceptable salts thereof, wherein each R₁ is independently hydrogen or methyl; R is naphthyl or 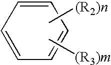
wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropyl amine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropyl amine bromide, N,N-dimethyl 3-(B-naphthoxy)-3-phenyl-1-methylpropylamine iodide, 3-(2′-methyl-4′,5 ′-dichlorophenoxy)-3-phenylpropylamine nitrate, 3-(p)-t-butylphenoxy)-3-phenylpropyl amine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine maleate (LUVOX™) is chemically designated as 5-methoxy-4′-(trifluoromethyl)valerophenone (E)-O-(2-aminoethyl)oxime maleate. Fluvoxamine maleate is supplied as 50 mg and 100 mg tablets.

Fluvoxamine has the following structure:

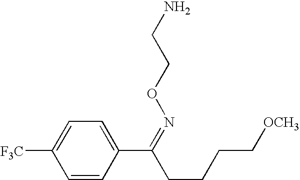

Structural analogs of fluvoxamine are those having the formula: 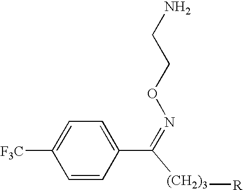
as well as pharmaceutically acceptable salts thereof, wherein R is cyano, cyanomethyl, methoxymethyl, or ethoxymethyl.
Indalpine

Indalpine has the following structure:

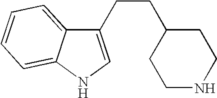

Structural analogs of indalpine are those having the formula: 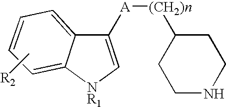
or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3(piperidyl-4 methyl) ketone; (methoxy-5-indolyl-3) (piperidyl-4 methyl)ketone; (chloro-5-indolyl-3) (piperidyl-4 methyl)ketone; (indolyl-3)-l(piperidyl-4)-3 propanone, indolyl-3 piperidyl-4 ketone; (methyl-1 indolyl-3) (piperidyl-4 methyl)ketone, (benzyl-1 indolyl-3) (piperidyl-4 methyl)ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxezine has the following structure:

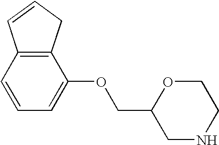

Structural analogs of indeloxazine are those having the formula: 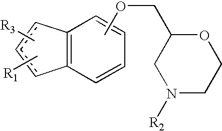
and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(l-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipram (IXEL™, Cypress Bioscience Inc.) has the chemical formula (Z)-1-diethylaminocarbonyl-2-aminoethyl-1-phenyl-cyclopropane)hydrochlorate, and is provided in 25 mg and 50 mg tablets for oral administration.

Milnacipram has the following structure:

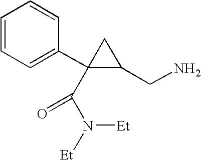

Structural analogs of milnacipram are those having the formula: 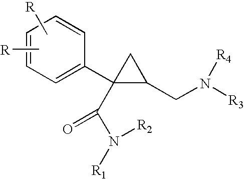
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine hydrochloride((−)-trans-4 R-(4′-fluorophenyl)-3 S-[(3′,4′-methylenedioxyphenoxy) methyl]piperidine hydrochloride hemihydrate) is provided as PAXIL™. Controlled-release tablets contain paroxetine hydrochloride equivalent to paroxetine in 12.5 mg, 25 mg, or 37.5 mg dosages. One layer of the tablet consists of a degradable barrier layer and the other contains the active material in a hydrophilic matrix.

Paroxetine has the following structure:

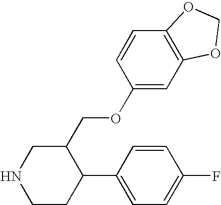

Structural analogs of paroxetine are those having the formula: 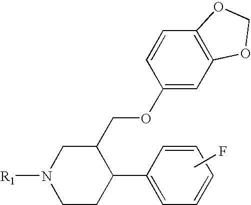
and pharmaceutically acceptable salts thereof, wherein R₁ represents hydrogen or a C₁₋₄ alkyl group, and the fluorine atom may be in any of the available positions.

Sertraline

Sertraline ((1S-cis)-4-(3,4-dichlorophenyl)-1,2,3,4-tetrahydro-N-methyl-1-nanphthalenamine hydrochloride) is provided as ZOLOFT™ in 25 mg, 50 mg and 100 mg tablets for oral administration. Because sertraline undergoes extensive metabolic transformation into a number of metabolites that may be therapeutically active, these metabolites may be substituted for sertraline in a drug combination described herein. The metabolism of sertraline includes, for example, oxidative N-demethylation to yield N-desmethylsertraline (nor-sertraline).

Sertraline has the following structure:

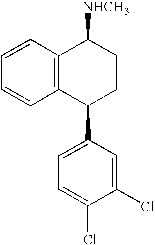

Structural analogs of sertraline are those having the formula: 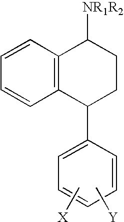
wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e., they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Sibutramine Hydrochloride Monohydrate

Sibutramine hydrochloride monohydrate (MERIDIA™) is an orally administered agent for the treatment of obesity. Sibutramine hydrochloride is a racemic mixture of the (+) and (−) enantiomers of cyclobutanemethanamine, 1-(4-chlorophenyl)-N, N-dimethyl-(alpha)-(2-methylpropyl)-, hydrochloride, monohydrate. Each MERIDIA™ capsule contains 5 mg, 10 mg, or 15 mg of sibutramine hydrochloride monohydrate.

Zimeldine

Zimeldine has the following structure:

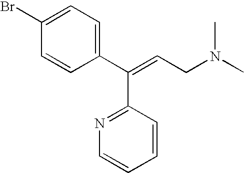

Structural analogs of zimeldine are those compounds having the formula: 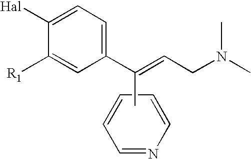
and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be employed in any of the drug combinations described herein.

Metabolites

Pharmacologically active metabolites of any of the foregoing SSRIs can also be used in the drug combinations described herein. Exemplary metabolites are didesmethylcitalopram, desmethylcitalopram, desmethylsertraline, and norfluoxetine.

Analogs

Functional analogs of SSRIs can also be used in the drug combinations described herein. Exemplary SSRI functional analogs are provided below. One class of SSRI analogs are SNRIs (selective serotonin norepinephrine reuptake inhibitors), which include venlafaxine and duloxetine.

Venlafaxine

Venlafaxine hydrochloride (EFFEXOR™) is an antidepressant for oral administration. It is designated (R/S)-1-[2-(dimethylamino)-1-(4-methoxyphenyl)ethyl]cyclohexanol hydrochloride or (±)-1-[(alpha)-[(dimethyl-amino)methyl]-p-methoxybenzyl]cyclohexanol hydrochloride.

Venlafaxine has the following structure:

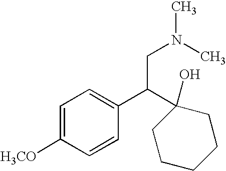

Structural analogs of venlafaxine are those compounds having the formula: 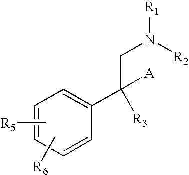
as well as pharmaceutically acceptable salts thereof, wherein A is a moiety of the formula: 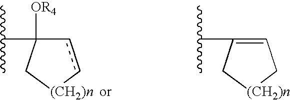
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

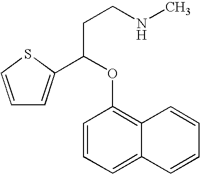

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno [2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO04/004734.

Tricyclic Antidepressants

In another embodiment, a drug combination comprises a tricyclic antidepressant (TCA) (which are described herein in detail), or a structural or functional analog thereof in combination with a non-steroidal immunophilin-dependent immunosuppressant (NsIDI). Maprotiline (brand name LUDIOMIL) is a secondary amine tricyclic antidepressant that inhibits norepinephrine reuptake and is structurally related to imipramine, a dibenzazepine. While such agents have been used for the treatment of anxiety and depression, maprotiline, for example, increases the potency of an immunosuppressive agent, and is useful as anti-inflammatory agent.

Maprotiline (brand name LUDIOMIL) and maprotiline structural analogs have three-ring molecular cores (see formula (IV), supra). These analogs include other tricyclic antidepressants (TCAs) having secondary amine side chains (e.g., nortriptyline, protriptyline, desipramine) as well as N-demethylated metabolites of TCAs having tertiary amine side chains. Preferred maprotiline structural and functional analogs include tricyclic antidepressants that are selective inhibitors of norepinephrine reuptake. Tricyclic compounds that can be used in the methods, compositions, and kits of the invention include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. Pat. application Ser. Nos. 10/617,424 or 60/504,310.

Triclosan

In another embodiment, a drug combination comprises triclosan or another phenoxy phenol, or a structural or functional analog thereof in combination with a non-steroidal immunophilin-dependent immunosuppressant (NsIDI).

Triclosan is a chloro-substituted phenoxy phenol that acts as a broad-spectrum antibiotic. We report herein that triclosan also increases the potency of immunosuppressive agents, such as cyclosporine, and is useful in the anti-inflammatory combination of the invention for the treatment of an immunoinflammatory disorder, proliferative skin disease, organ transplant rejection, or graft versus host disease. Triclosan structural analogs include chloro-substituted phenoxy phenols, such as 5-chloro-2-(2,4-dichlorophenoxy)phenol, hexachlorophene, dichlorophene, as well as other halogenated hydroxydiphenyl ether compounds. Triclosan functional analogs include clotrimazole as well as various antimicrobials such as selenium sulfide, ketoconazole, triclocarbon, zinc pyrithione, itraconazole, asiatic acid, hinokitiol, mipirocin, clinacycin hydrochloride, benzoyl peroxide, benzyl peroxide, minocyclin, octopirox, ciclopirox, erythromycin, zinc, tetracycline, azelaic acid and its derivatives, phenoxy ethanol, ethylacetate, clindamycin, meclocycline. Functional and/or structural analogs of triclosan are also described, e.g., in U.S. Pat. Nos. 5,043,154, 5,800,803, 6,307,049, and 6,503,903.

Triclosan may achieve its anti-bacterial activity by binding to and inhibiting the bacterial enzyme Fab1, which is required for bacterial fatty acid synthesis. Triclosan structural or functional analogs, including antibiotics that bind Fab1, may also be useful in the combinations of the invention.

Antihistamines

In yet another embodiment a drug combination comprises a histamine receptor antagonist (or analog thereof) and a non-steroidal immunophilin-dependent inhibitor. Antihistamines are compounds that block the action of histamine. Classes of antihistamines include the following:

(1) Ethanolamines (e.g., bromodiphenhydramine, carbinoxamine, clemastine, dimenhydrinate, diphenhydramine, diphenylpyraline, and doxylamine); (2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) Piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the drug combinations described herein, either non-sedating or sedating antihistamines may be employed. Particularly desirable antihistamines for use in the drug combinations described herein are non-sedating antihistamines such as loratadine and desloratadine. Sedating antihistamines can also be used in a drug combination. In certain embodiments, sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other suitable antihistamines include acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in a drug combination described herein. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10-H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(1011-dihydro-5H-dibenzo(a,d)cyclohepten-5-ylidene)-tropane.

Other suitable compounds for use in a drug combination include AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the drug combinations described herein are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Loratadine

Loratadine (CLARITIN) is a tricyclic piperidine that acts as a selective peripheral histamine H1-receptor antagonist. Loratadine and structural and functional analogs thereof, such as piperidines, tricyclic piperidines, histamine H1-receptor antagonists, are useful in a drug combination described herein.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Phenothiazines

In another embodiment, the drug combination comprises a phenothiazine, or a structural or functional analog thereof, in combination with a non-steroidal immunophilin-dependent immunosuppressant (NsIDI).

Phenothiazines that are useful in the drug combinations include compounds having the general formula (VI): 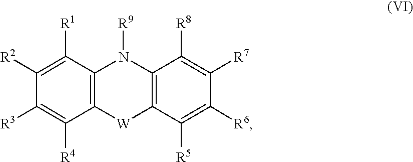
or a pharmaceutically acceptable salt thereof, wherein R² is selected from the group consisting of: CF₃, Cl, F, OCH₃, COCH₃, CN, OCF₃, COCH₂CH₃, CO(CH₂)₂CH₃, and SCH₂CH₃; R⁹ is selected from the group consisting of: 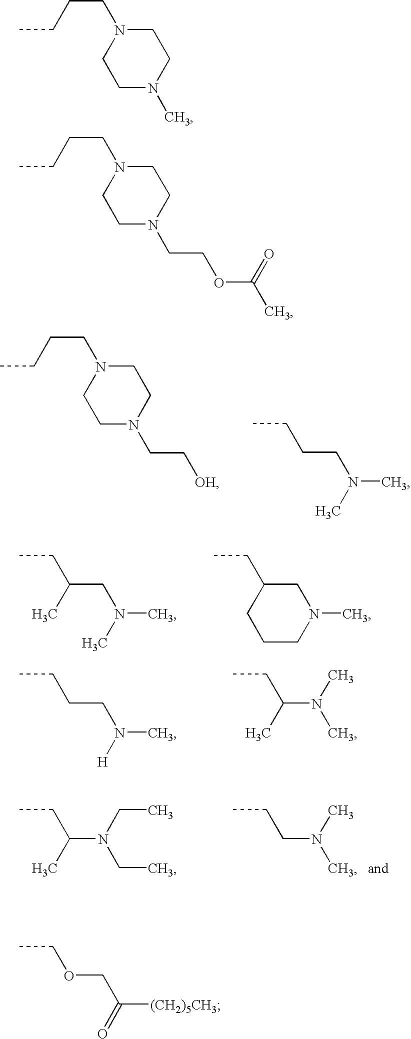
each of R¹, R³, R⁴, R⁵, R⁶, R⁷, and R⁸ is, independently, H, OH, F, OCF₃, or OCH₃; and W is selected from the group consisting of:

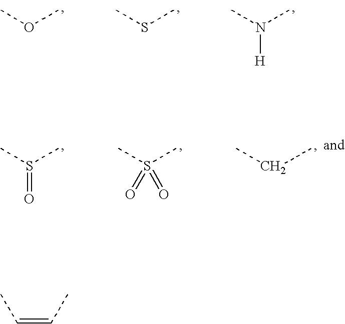

In some embodiments, the phenothiazine is a phenothiazine conjugate including a phenothiazine covalently attached via a linker to a bulky group of greater than 200 daltons or a charged group of less than 200 daltons. Such conjugates retain their anti-inflammatory activity in vivo and have reduced activity in the central nervous system in comparison to the parent phenothiazine.

Phenothiazine conjugates that are useful in drug combinations described herein include compounds having the general formula (VII).

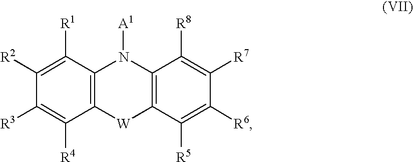

In formula (VII), R² is selected from the group consisting of: CF₃, halo, OCH₃, COCH₃, CN, OCF₃, COCH₂CH₃, CO(CH₂)₂CH₃, S(O)₂CH₃, S(O)₂N(CH₃)₂, and SCH₂CH₃; A¹ is selected from the group consisting of G¹, 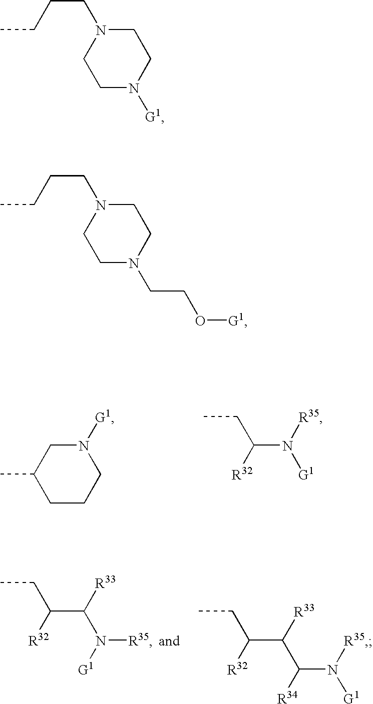
each of R¹, R³, R⁴, R⁵, R⁶, R⁷, and R⁸ is independently H, OH, F, OCF₃, or OCH₃; R³², R³³, R³⁴, and R³⁵, are each, independently, selected from H or C₁₋₆ alkyl; W is selected from the group consisting of: NO, 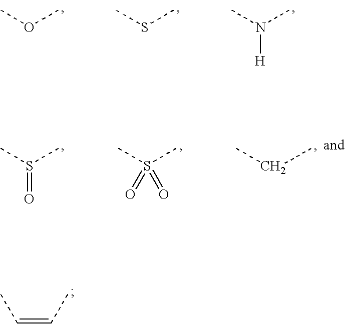
and G¹ is a bond between the phenothiazine and a linker, L.

The linker L is described by formula (VIII):
G¹-(Z¹)ₒ-(Y¹)ᵤ-(Z²)ₛ-(R⁹)-(Z3)ₜ-(Y²)ᵥ-(z⁴)ₚ-G² (VIII)

In formula (VIII), G¹ is a bond between the phenothiazine and the linker, G² is a bond between the linker and the bulky group or between the linker and the charged group, each of Z¹, Z², Z³, and Z⁴ is, independently, selected from O, S, and NR³⁹; R³⁹ is hydrogen or a C₁₋₆ alkyl group; each of Y¹ and Y² is, independently, selected from carbonyl, thiocarbonyl, sulphonyl, phosphoryl or similar acid-forming groups; o, p, s, t, u, and v are each independently 0 or 1; and R⁹ is a C₁₋₁₀ alkyl, a linear or branched heteroalkyl of 1 to 10 atoms, a C₂₋₁₀ alkene, a C₂₋₁₀ alkyne, a C₅₋₀ aryl, a cyclic system of 3 to 10 atoms, —(CH₂CH₂O)_{q}CH₂CH₂— in which q is an integer of 1 to 4, or a chemical bond linking G¹-(Z¹)ₒ-(Y¹)ᵤ-(Z²)ₛ- to -(Z³)ₜ-(Y²)ᵥ-(Z⁴)ₚ-G².

The bulky group can be a naturally occurring polymer or a synthetic polymer. Natural polymers that can be used include, without limitation, glycoproteins, polypeptides, or polysaccharides. Desirably, when the bulky group includes a natural polymer, the natural polymer is selected from alpha-1-acid glycoprotein and hyaluronic acid. Synthetic polymers that can be used as bulky groups include, without limitation, polyethylene glycol, and the synthetic polypeptide N-hxg.

The most commonly prescribed member of the phenothiazine family is chlorpromazine, which has the structure:

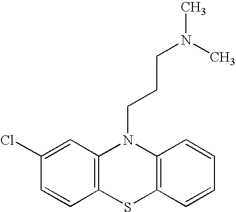

Chlorpromazine is a phenothiazine that has long been used to treat psychotic disorders. Phenothiazines include chlorpromazine functional and structural analogs, such as acepromazine, chlorfenethazine, chlorpromazine, cyamemazine, enanthate, fluphenazine, mepazine, mesoridazine besylate, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, or triflupromazine (or a salt of any of the above); and functional analogs that act as dopamine D2 antagonists (e.g., sulpride, pimozide, spiperone, clebopride, bupropion, and haloperidol).

Chlorpromazine is currently available in the following forms: tablets, capsules, suppositories, oral concentrates and syrups, and formulations for injection.

Because chlorpromazine undergoes extensive metabolic transformation into a number of metabolites that may be therapeutically active, these metabolites may be substituted for chlorpromazine in a drug combination described herein. The metabolism of chlorpromazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide. In other examples of chlorpromazine metabolites useful in the anti-inflammatory combination of the invention, each of positions 3, 7, and 8 of the phenothiazine can independently be substituted with a hydroxyl or methoxyl moiety.

Another phenothiazine is ethopropazine (brand name PARSITAN), an anticholinergic phenothiazine that is used as an antidyskinetic for the treatment of movement disorders, such as Parkinson's disease. Ethopropazine also has antihistaminic properties. Ethopropazine also increases the potency of immunosuppressive agents, such as cyclosporines. Unlike antipsychotic phenothiazines, which have three carbon atoms between position 10 of the central ring and the first amino nitrogen atom of the side chain at this position, strongly anticholinergic phenothiazines (e.g., ethopropazine, diethazine) have only two carbon atoms separating the amino group from position 10 of the central ring.

Ethopropazine structural analogs include trifluoroperazine dihydrochloride, thioridazine hydrochloride, and promethazine hydrochloride. Additional ethopropapazine structural analogs include 10-[2,3-bis(dimethylamino)propyl]phenothiazine, 10-[2,3-bis(dimethylamino)propyl]phenothiazine hydrochloride, 10-[2-(dimethylamino)propyl]phenothiazine; 10-[2-(dimethylamino)propyl]phenothiazine hydrochloride; and 10-[2-(diethylamino)ethyl]phenothiazine and mixtures thereof (see, e.g., U.S. Pat. No. 4,833,138).

Ethopropazine acts by inhibiting butyrylcholinesterase. Ethopropazine functional analogs include other anticholinergic compounds, such as Artane (trihexyphenidyl), Cogentin (benztropine), biperiden (U.S. Pat. No. 5,221,536), caramiphen, ethopropazine, procyclidine (Kemadrin), and trihexyphenidyl. Anticholinergic phenothiazines are extensively metabolized, primarily to N-dealkylated and hydroxylated metabolites. Ethopropazine metabolites may be substituted for ethopropazine in the drug combinations described herein.

Mu Opioid Receptor Agonists

In yet another embodiment, a drug combination may comprise a mu opioid receptor agonist (or analog thereof) and a non-steroidal immunophilin-dependent inhibitor. Loperamide hydrochloride (IMMODIUM) is a mu opioid receptor agonist useful in the treatment of diarrhea (U.S. Pat. No. 3,714,159). Loperamide and loperamide analogs increase the potency of an immunosuppressive agent and are useful in the treatment of an immunoinflammatory disorder, organ transplant rejection, or graft versus host disease. Loperamide is a piperidine butyramide derivative that is related to meperidine and diphenoxylate. It acts by relaxing smooth muscles and slowing intestinal motility. Other functionally and/or structurally related compounds, include meperidine, diphenoxylate, and related propanamines. Additional loperamide functional and structural analogs are described, e.g., in U.S. Pat. Nos. 4,066,654, 4,069,223, 4,072,686, 4,116,963, 4,125,531, 4,194,045, 4,824,853, 4,898,873, 5,143,938, 5,236,947, 5,242,944, 5,849,761, and 6,353,004. Loperamide functional analogs include peptide and small molecule mu opioid receptor agonists (described in U.S. Pat. No. 5,837,809). Such agents are also useful in the drug combinations described herein. Loperamide is capable of binding to opioid receptors within the intestine and altering gastrointestinal motility.

Corticosteroids

In certain embodiments, the drug combinations described herein may be used with additional therapeutic agents, including corticosteroids. One or more corticosteroid may be formulated with non-steroidal immunophilin-dependent enhancer, or analog or metabolite thereof, in a drug combination described herein. Suitable corticosteroids are described in detail herein. Corticosteroid compounds that may be included in the drug combination containing a non-steroidal immunophilin-dependent enhancer include any one of the corticosteroids described in detail herein and known in the art.

Steroid Receptor Modulators

In still other embodiments, a drug combination ma comprise a steroid receptor modulator (e.g., an antagonist or agonist) as a substitute for or in addition to a corticosteroid. Thus, in one embodiment, the drug combination comprises an NsIDI (or an analog or metabolite thereof) and an NsIDIE and, optionally, a glucocorticoid receptor modulator or other steroid receptor modulator.

Glucocorticoid receptor modulators that may used are described in U.S. Pat. Nos. 6,380,207, 6,380,223, 6,448,405, 6,506,766, and 6,570,020, U.S. Patent Application Publication Nos. 20030176478, 20030171585, 20030120081, 20030073703, 2002015631, 20020147336, 20020107235, 20020103217, and 20010041802, and PCT Publication No. WO00/66522, each of which is hereby incorporated by reference. Other steroid receptor modulators are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used in combination with a NsIDI/NsIDIE in the drug combinations described herein include, for example, A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffinann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

In one embodiment, one or more agents typically used to treat COPD may be used as a substitute for or in addition to an NSIDI in the drug combination described herein. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, a drug combination comprises a tricyclic compound and a bronchodilator.

In a certain embodiment, one or more antipsoriatic agents typically used to treat psoriasis may be used as a substitute for or in addition to an NSIDI in the drug combination described herein. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, a drug combination features the combination of a tricyclic compound and an antipsoriatic agent.

In yet another embodiment, one or more agents typically used to treat inflammatory bowel disease may be used as a substitute for or in addition to an NsIDI in the drug combinations described herein. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, a drug combination features the combination of a tricyclic compound and any of the foregoing agents.

In still another embodiment, one or more agents typically used to treat rheumatoid arthritis may be used as a substitute for or in addition to an NsIDI in the drug combination described herein. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, a drug combination features the combination of a tricyclic compound with any of the foregoing agents.

In another embodiment, one or more agents typically used to treat asthma may be used as a substitute for or in addition to an NsIDI in the drug combination described herein. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, a drug combination features the combination of a tricyclic compound and any of the foregoing agents.

An NsIDI and an NsIDIE may be combined with other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies may be useful for the treatment of inflammatory disorders or diseases in combination with other anti-cytokine agents or agents that modulate the immune response to positively treat or prevent disease, such as agents that influence cell adhesion, or biologics (i.e., agents that block the action of IL-6, IL-1, IL-2, IL-12, IL-15 or TNF (e.g., etanercept, adelimumab, infliximab, or CDP-870). Without wishing to be bound by theory, when using agents that block the effect of TNFα, a combination therapy reduces the production of cytokines, and then agents such as etanercept or infliximab act on the remaining fraction of inflammatory cytokines, providing enhanced treatment.

Accordingly, provided herein is a drug combination comprising a non-steroidal immunophilin-dependent immunosuppressant (NsIDI) and an NsIDI enhancer (NsIDIE). Such a drug combination may also exhibit a biological activity such as the capability to decrease proinflammatory cytokine secretion or production and/or to prevent or treat an inflammatory response and/or treat or prevent an immunological disease or disorder such as an inflammatory disease or disorder or an autoimmune disease or disorder. In a particular embodiment, the NsIDI is a calcineurin inhibitor; and in another particular embodiment, the calcineurin inhibitor is cyclosporine, tacrolimus, ascomycin, pimecrolimus, or ISAtx247. In another embodiment, the NsIDI is an FK506-binding protein, which in certain specific embodiments is rapamycin or everolimus. In other embodiments, the NsIDIE is a selective serotonin reuptake inhibitor (SSRI), a tricyclic antidepressant (TCA), a phenoxy phenol, an antihistamine, a phenothiazine, or a mu opioid receptor agonist. In a particular embodiment, the SSRI is selected from fluoxetine, sertraline, paroxetine, fluvoxamine, citalopram, and escitalopram. In another certain embodiment, the TCA is selected from maprotiline, nortriptyline, protriptyline, desipramine, amitriptyline, amoxapine, clomipramine, dothiepin, doxepin, desipramine, imipramine, lofepramine, mianserin, oxaprotiline, octriptyline, and trimipramine. In a particular specific embodiment, the phenoxy phenol is triclosan. In another particular embodiment, the antihistamine is selected from ethanolamines, ethylenediamines, phenothiazines, alkylamines, piperazines, piperidines, and atypical antihistamines. In another embodiment, the antihistamine is selected from desloratadine, thiethylperazine, bromodiphenhydramine, promethazine, cyproheptadine, loratadine, clemizole, azatadine, cetirizine, chlorpheniramine, dimenhydramine, diphenydra mine, doxylamine, fexofenadine, meclizine, pyrilamine, and tripelennamine.

In other particular embodiments, the phenothiazine is chlorpromazine or ethopropazine. In another particular embodiment, the mu opioid receptor agonist is a piperidine butyramide derivative. In certain other embodiments, the mu opioid receptor agonist is loperamide, meperidine, or diphenoxylate. In a specific embodiment, the drug combination comprises an NSIDI that is cyclosporine (e.g., cyclosporine A) and a mu opiod receptor loperamide. In another embodiment the drug combination comprises cyclosporine and the antihistamine ethopropazine. In yet other specific embodiments, the drug combination comprises cyclosporine and any one of the following agents: chlorpromazine, loratadine, desloratadine, triclosan (a phenoxy phenol), maprotiline (a TCA), paroxetine (an SSRI), fluoxetine (an SSRI), or sertraline (an SSRI). In another specific embodiment, the NSIDI is tacrolimus (a calcineurin inhibitor) and fluvoxamine (an SSRI).

In other embodiments, the drug combination described herein further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal calcineurin inhibitor, vitamin D analog, psoralen, retinoid, or 5-amino salicylic acid. In a more particular embodiment, the NSAID is ibuprofen, diclofenac, or naproxen; and in another particular embodiment, the COX-2 inhibitor is rofecoxib, celecoxib, valdecoxib, or lumiracoxib. In still another certain embodiment, the biologic is adelimumab, etanercept, or infliximab. In another embodiment, the DMARD is methotrexate or leflunomide. In certain embodiments, xanthine is theophylline; the anticholinergic compound is ipratropium or tiotropium; the beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline; the vitamin D analog is calcipotriene or calcipotriol; the psoralen is methoxsalen; the retinoid is acitretin or tazoretene; the 5-amino salicylic acid is mesalamine, sulfasalazine, balsalazide disodium, or olsalazine sodium; and the small molecule immunomodulator is VX 702, SCIO 469, doramapimod, RO 30201195, SCIO 323, DPC 333, pranalcasan, mycophenolate, or merimepodib.

Drug Combination Comprising an Antihistamine and Additional Agents

In another embodiment, the drug combination that has anti-scarring activity comprises at least two agents, wherein at least one agent is an antihistamine, and at least one second agent is selected from a corticosteroid and any of a number of additional agents described herein.

In another embodiment, the drug combination includes an antihistamine and a corticosteroid. In certain embodiments, the antihistamine is bromodiphenhydramine, clemizole, cyproheptadine, desloratadine, loratadine, thiethylperazine maleate, or promethazine. In certain embodiments, the corticosteroid is prednisolone, cortisone, dexamethasone, hydrocortisone, methylprednisolone, fluticasone, prednisone, triamcinolone, or diflorasone. In still other embodiments, the drug combination further comprises at least one (i.e., one or more) additional compounds, including but not limited to a glucocorticoid receptor modulator, NSAID, COX-2 inhibitor, DMARD, biologic, small molecule immunomodulator, xanthine, anticholinergic compound, beta receptor agonist, bronchodilator non-steroidal immunophilin-dependent immunosuppressant, vitamin D analog, psoralen, retinoid, or 5-amino salicylic acid.

In a particular embodiment, a drug combination comprises an antihistamine and ibudilast, and in another particular embodiment, the drug combination comprises an antihistamine and rolipram. In still another specific embodiment, the drug combination comprises an antihistamine and a tetra-substituted pyrimidopyrimidine, wherein in certain embodiments, the tetra-substituted pyrimidopyrimidine is dipyridamole. In another specific embodiment, the drug combination comprises an antihistamine and a tricyclic or tetracyclic antidepressant. In other specific embodiments, the tricyclic or tetracyclic antidepressant is nortryptiline, amoxapine, or desipramine. In one embodiment, the antihistamine is not doxepin, while in another embodiment, the antidepressant is not doxepin. In yet another embodiment, a drug combination comprises an antihistamine and a selective serotonin reuptake inhibitor (SSRI). In certain embodiments, the antihistamine is selected from bromodiphenhydramine, clemizole, cyproheptadine, desloratadine, loratadine, thiethylperazine maleate, and promethazine, and the SSRI is selected from paroxetine, fluoxetine, sertraline, and citalopram.

As described in detail herein, by “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be h alogenated. Exemplary corticosteroids are described herein.

By “tricyclic or tetracyclic antidepressant” is meant a compound having one the formulas (I), (II), (III), or (IV), which are described in greater detail herein.

By “antihistamine” is meant a compound that blocks the action of histamine. Classes of antihistamines include but are not limited to, ethanolamines, ethylenediamine, phenothiazine, alkylamines, piperazines, and piperidines.

By “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are fluoxetine, fluvoxamine, paroxetine, sertraline, citalopram, and venlafaxine.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which binds to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

In one embodiment, a drug combination comprises an antihistamine (or analog thereof) and a corticosteroid. In another embodiment, a drug combination comprises an antihistamine (or analog thereof) and a tricyclic or tetracyclic antidepressant. In yet another embodiment, a drug combination comprises an antihistamine (or analog thereof) and a selective serotonin reuptake inhibitor. In still other embodiments, a drug combination comprises an antihistamine or antihistamine analog, and dipyridamole, ibudilast, and/or rolipram, or an analog of any of these compounds.

Antihistamines

As described in detail herein, antihistamines, as described herein and above, are compounds that block the action of histamine. Classes of antihistamines include the following:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) Piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the drug combinations described herein, either non-sedating or sedating antihistamines may be employed. In certain embodiments, antihistamines for use in the drug combinations described herein are non-sedating antihistamines such as loratadine and desloratadine. Sedating antihistamines can also be used in a drug combination. In certain embodiments, sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the drug combinations described herein are acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in according to the invention. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10-H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(1O,11-dihydro-5H-dibenzo(a,d)cyclohepten-5-ylidene)-tropane.

Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the invention are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Loratadine

Loratadine (CLARITIN) is a tricyclic piperidine that acts as a selective peripheral histamine H1-receptor antagonist. Loratadine and structural and functional analogs thereof, such as piperidines, tricyclic piperidines, histamine H1-receptor antagonists, may be used in the drug combinations described herein.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Corticosteroids

In certain embodiments, one or more corticosteroid may be combined and formulated with an antihistamine or analog thereof in a drug combination described herein. Various antihistamines in combination with various corticosteroids are more effective in suppressing TNFα in vitro than either agent alone. Corticosteroids are described in detail herein and suitable corticosteroids for use in combination with an anti-histamine include any one of the corticosteroid compounds described herein.

Steroid Receptor Modulators

Steroid receptor modulators (e.g., antagonists and agonists) may be used as a substitute for or in addition to a corticosteroid in the drug combinations described herein. Thus, in one embodiment, the invention features the combination of a tricyclic compound and a glucocorticoid receptor modulator or other steroid receptor modulator.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Ibudilast

In one embodiment, a drug combination comprises an antihistamine and ibudilast. Among the biological activities of such a drug combination includes the capability to suppress TNFα in vitro more effectively than either agent alone.

Ibudilast, or an ibudilast analog, has a structure of formula (IX).

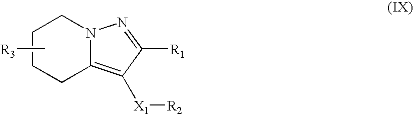

In formula (IX) R₁ and R₂ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₃ is selected from H, halide, alkoxy, and C₁₋₄ alkyl; X₁ is selected from C═O, C═N—NH—R₄, C═C(R₅)—C(O)-R₆, C═CH═CH—C(O)—R₆, and C(OH)—R₇; R₄ is selected from H and acyl; R₅ is selected from H, halide, and C₁₋₄ alkyl; R₆ is selected from OH, alkoxy and amido; and R₇ is selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. Compounds of formula (IX) include, the compounds described in U.S. Pat. Nos. 3,850,941; 4,097,483; 4,578,392; 4,925,849; 4,994,453; and 5,296,490. Commercially available compounds of formula (IX) include ibudilast and KC-764.

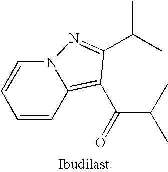

KC-764 (CAS 94457-09-7) is reported to be a platelet aggregation inhibitor.

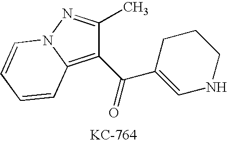

KC-764 and other compound of formula (IX) can be prepared using the synthetic methods described in U.S. Pat. Nos. 3,850,941; 4,097,483; 4,578,392; 4,925,849; 4,994,453; and 5,296,490.

Rolipram

In another embodiment, a drug combination comprises an antihistamine, or an analog thereof, and rolipram (4-[3-(cyclopentyloxy)-4-methoxyphenyl]-2-pyrrolidone) or an analog of rolipram. Rolipram analogs are described by formula (I) of U.S. Pat. No. 4,193,926, hereby incorporated by reference.

Tetra-substituted Pyrimidopyrimidines

In another embodiment, a drug combination is provided that comprises an antihistamine, or analog thereof, in combination with a tetra-substituted pyrimidopyrimidine such as dipyridamole.

A tetra-substituted pyrimidopyrimidine comprises a structure having the formula (V) as described in detail herein. Exemplary tetra-substituted pyrimidopyrimidines that are useful in the drug combinations and methods described herein include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. No. 3,031,450, hereby incorporated by reference.

Tricyclic and Tetracyclic Antidepressants

In another embodiment, the drug combination comprises an antihistamine or antihistamine analog in combination with tricyclic and tetracyclic antidepressants and their analogs.

In one embodiment of the invention, an antihistamine or analog thereof is administered or formulated with a tricyclic or tetracyclic antidepressant, or an analog thereof. By “tricyclic or tetracyclic antidepressant analog” is meant a compound having one the formulas (I), (II), (III), or (IV), which are described in detail herein.

Tricyclic or tetracyclic antidepressants, as well as analogs thereof, that are suitable for use in the drug combinations described herein include 10-(4-methylpiperazin-1-yl)pyrido(4,3-b)(1,4)benzothiazepine; 11-(4-methyl-1-piperazinyl)-5H -dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; 8-chloro-2-methoxy-11-(4-methyl-1-piperazinyl)-5H-dibenzo(b,e)(1,4)diazepine; (Z)-2-butenedioate; 7-hydroxyamoxapine; 8-hydroxyamoxapine; 8-hydroxyloxapine; Adinazolam; Amineptine; amitriptyline; amitriptylinoxide; amoxapine; butriptyline; clomipramine; clothiapine; clozapine; demexiptiline; desipramine; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; 11-(4-methyl-1-piperazinyl)-dibenzo(b,f)(1,4)thiazepine; dibenzepin; dimetacrine; dothiepin; doxepin; fluacizine; fluperlapine; imipramine; imipramine N-oxide; iprindole lofepramine; loxapine; loxapine hydrochloride; loxapine succinate; maprotiline; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; nortriptyline; noxiptilin; octriptyline; opipramol; oxaprotiline; perlapine; pizotyline; propizepine; protriptyline; quetiapine; quinupramine; tianeptine; tomoxetine; and trimipramine. Others are described in U.S. Pat. Nos. 4,933,438 and 4,931,435.

Selective Serotonin Reuptake Inhibitors

In another embodiment, a drug combination provided herein comprises an antihistamine or analog thereof in combination with any one of a number of SSRI compounds, or analog thereof, described herein and available in the art.

As described herein, suitable SSRIs and SSRI analogs include 1,2,3,4-tetrahydro-N-methyl-4-phenyl-1-naphthylamine hydrochloride, 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; cericlaimine; citalopram; xitalopram hydrobromide; CP 53261; didesmethylcitalopram; escitalopram; escitalopram oxalate; femoxetine, fluoxetine; fluoxetine hydrochloride; fluvoxamine; fluvoxamine maleate; indalpine, indeloxazine hydrochloride, Lu 19005; milnacipram; monodesmethylcitalopram; N-(3-fluoropropyl)paroxetine; norfluoxetine; O-desmethylvenlafaxine; paroxetine; paroxetine hydrochloride; paroxetine maleate; sertraline; sertraline hydrochloride; tametraline hydrochloride; venlafaxine; venlafaxine hydrochloride; WY 45,818; WY 45,881, and zimeldine. Other SSRI or SSRI analogs useful in the methods and compositions of the invention are described in U.S. Pat. Nos. 3,912,743; 4,007,196; 4,136,193; 4,314,081; and 4,536,518, each hereby incorporated by reference.

Citalopram

Citalopram HBr (CELEXA™) is a racemic bicyclic phthalane derivative designated (±)-1-(3-dimethylaminopropyl)-1-(4-fluorophenyl)-1,3-dihydroisobenzofuran-5-carbonitrile, HBr. Citalopram undergoes extensive metabolization; nor₁-citalopram and nor₂-citalopram are the main metabolites. Citalopram is available in 10 mg, 20 mg, and 40 mg tablets for oral administration. CELEXA™ oral solution contains citalopram HBr equivalent to 2 mg/mL citalopram base. CELEXA™ is typically administered at an initial dose of 20 mg once daily, generally with an increase to a dose of 40 mg/day. Dose increases typically occur in increments of 20 mg at intervals of no less than one week.

Citalopram has the following structure:

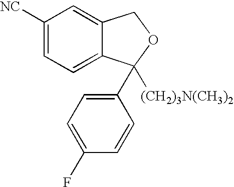

Structural analogs of citalopram are those having the formula: 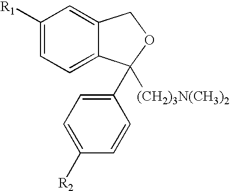
as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Clovoxamine has the following structure:

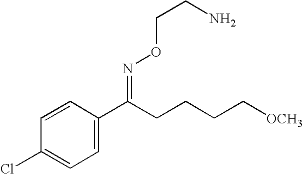

Structural analogs of clovoxamine are those having the formula: 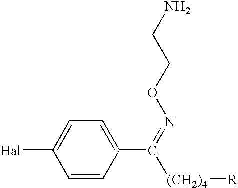
as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

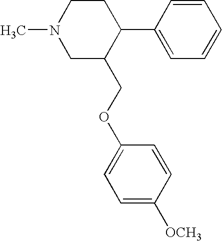

Structural analogs of femoxetine are those having the formula: 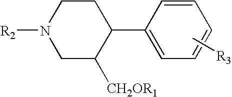
wherein R₁ represents a C₁₋₄-alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine hydrochloride ((±)-N-methyl-3-phenyl-3-[((alpha),(alpha),(alpha)-trifluoro-p-tolyl)oxy]propylamine hydrochloride) is sold as PROZAC™ in 10 mg, 20 mg, and 40 mg tablets for oral administration. The main metabolite of fluoxetine is nor-fluoxetine. By way of background, fluoxetine hydrochloride is typically administered as an oral solution equivalent to 20 mg/5 mL of fluoxetine. A delayed release formulation contains enteric-coated pellets of fluoxetine hydrochloride equivalent to 90 mg of fluoxetine. A dose of 20 mg/day, administered in the morning, is typically recommended as the initial dose. A dose increase may be considered after several weeks if no clinical improvement is observed.

Fluoxetine has the following structure:

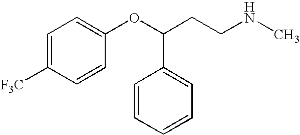

Structural analogs of fluoxetine are those compounds having the formula: 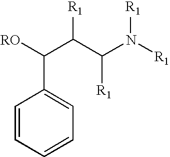
as well as pharmaceutically acceptable salts thereof, wherein each R₁ is independently hydrogen or methyl; R is naphthyl or 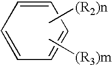
wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine maleate (LUVOX™) is chemically designated as 5-methoxy-4′-(trifluoromethyl)valerophenone (E)-O-(2-aminoethyl)oxime maleate. By way of background, fluvoxamine maleate is supplied as 50 mg and 100 mg tablets. Treatment for approved indications is typically initiated at 50 mg given once daily at bedtime, and then increased to 100 mg daily at bedtime after a few days, as tolerated. The effective daily dose usually lies between 100 and 200 mg, but may be administered up to a maximum of 300 mg.

Fluvoxamine has the following structure:

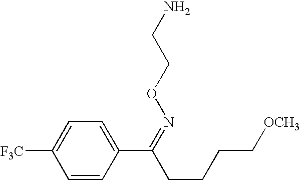

Structural analogs of fluvoxamine are those having the formula: 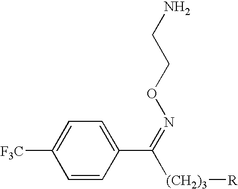
as well as pharmaceutically acceptable salts thereof, wherein R is cyano, cyanomethyl, methoxymethyl, or ethoxymethyl.
Indalpine

Indalpine has the following structure:

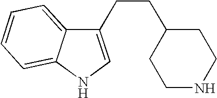

Structural analogs of indalpine are those having the formula: 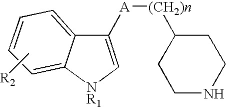
or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂group; and n is 0, 1 or 2.

Exemplary indalpine structural analogs are indolyl-3 (piperidyl-4 methyl) ketone; (methoxy-5-indolyl-3) (piperidyl-4 methyl)ketone; (chloro-5-indolyl-3) (piperidyl-4 methyl)ketone; (indolyl-3)-1 (piperidyl-4)-3 propanone, indolyl-3 piperidyl-4 ketone; (methyl-1 indolyl-3) (piperidyl-4 methyl)ketone, (benzyl-1 indolyl-3) (piperidyl-4 methyl)ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxezine has the following structure:

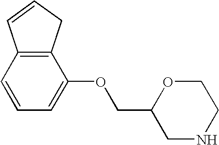

Structural analogs of indeloxazine are those having the formula: 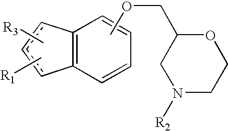
and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipram (IXEL™, Cypress Bioscience Inc.) has the chemical formula (Z)-1-diethylaminocarbonyl-2-aminoethyl-1-phenyl-cyclopropane)hydrochlorate, and is provided in 25 mg and 50 mg tablets for oral administration. By way of background, milnacipram is typically administered in dosages of 25 mg once a day, 25 mg twice a day, or 50 mg twice a day for the treatment of severe depression.

Milnacipram has the following structure:

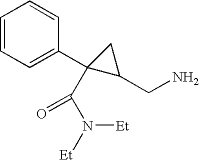

Structural analogs of milnacipram are those having the formula: 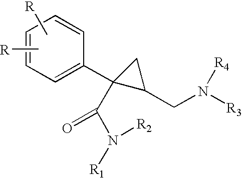
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine hydrochloride ((−)-trans-4 R-(4′-fluorophenyl)-3 S-[(3′,4′-methylenedioxyphenoxy)methyl]piperidine hydrochloride hemihydrate) is currently provided as PAXIL™. Controlled-release tablets contain paroxetine hydrochloride equivalent to paroxetine in 12.5 mg, 25 mg, or 37.5 mg dosages.

Paroxetine has the following structure:

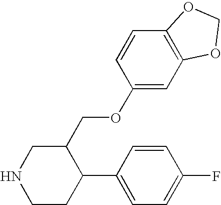

Structural analogs of paroxetine are those having the formula: 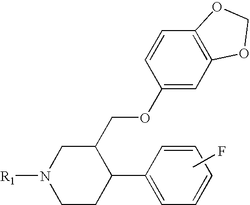
and pharmaceutically acceptable salts thereof, wherein R₁ represents hydrogen or a C₁₋₄ alkyl group, and the fluorine atom may be in any of the available positions.
Sertraline

Sertraline ((1S-cis)-4-(3,4-dichlorophenyl)-1,2,3,4-tetrahydro-N-methyl-1-nanphthalenamine hydrochloride) is provided as ZOLOFT™ in 25 mg, 50 mg and 100 mg tablets for oral administration. Because sertraline undergoes extensive metabolic transformation into a number of metabolites that may be therapeutically active, these metabolites may be substituted for sertraline in a drug combination described herein. The metabolism of sertraline includes, for example, oxidative N-demethylation to yield N-desmethylsertraline (nor-sertraline). ZOLOFT is typically administered at a dose of 50 mg once daily.

Sertraline has the following structure:

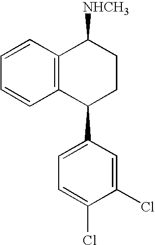

Structural analogs of sertraline are those having the formula: 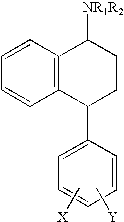
wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e., they are both oriented on the same side of the ring). Because both the 1- and 4-carbons are asymmetrically substituted, each cis- compound has two optically active enantiomeric forms denoted (with reference to the 1-carbon) as the cis-(1R) and cis-(1S) enantiomers.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1 R)-enantiomer of cis-N-methyl-4-(3,4-dichlorophenyl)-1,2,3,4-tetrahydro-1-naphthalenamine.

Sibutramine Hydrochloride Monohydrate

Sibutramine hydrochloride monohydrate (MERIDIA™) is an orally administered agent for the treatment of obesity. Sibutramine hydrochloride is a racemic mixture of the (+) and (−) enantiomers of cyclobutanemethanamine, 1-(4-chlorophenyl)-N,N-dimethyl-(alpha)-(2-methylpropyl)-, hydrochloride, monohydrate. Each MERIDIA™ capsule contains 5 mg, 10 mg, or 15 mg of sibutramine hydrochloride monohydrate.

Zimeldine

Zimeldine has the following structure:

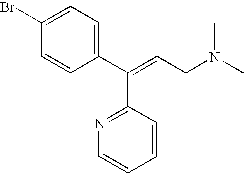

Structural analogs of zimeldine are those compounds having the formula: 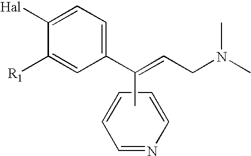
and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be used in any of the drug combinations described herein.

Metabolites

Pharmacologically active metabolites of any of the foregoing SSRIs can also be used in the drug combinations described herein. Exemplary metabolites are didesmethylcitalopram, desmethylcitalopram, desmethylsertraline, and norfluoxetine.

Analogs

Functional analogs of SSRIs can also be used in drug combinations described herein. Exemplary SSRI functional analogs are provided below. One class of SSRI analogs includes SNRIs (selective serotonin norepinephrine reuptake inhibitors), which include venlafaxine, duloxetine, and 4-(2-fluorophenyl)-6-methyl-2-piperazinothieno [2,3-d]pyrimidine.

Venlafaxine

Venlafaxine hydrochloride (EFFEXOR™) is an antidepressant for oral administration. It is designated (R/S)-1-[2-(dimethylamino)-1-(4-methoxyphenyl)ethyl]cyclohexanol hydrochloride or (±)-1-[(alpha)-[(dimethyl-amino)methyl]-p-methoxybenzyl]cyclohexanol hydrochloride. Compressed tablets contain venlafaxine hydrochloride equivalent to 25 mg, 37.5 mg, 50 mg, 75 mg, or 100 mg venlafaxine.

Venlafaxine has the following structure:

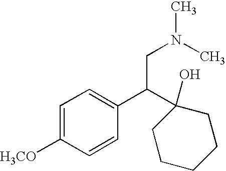

Structural analogs of venlafaxine are those compounds having the formula: 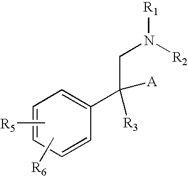
as well as pharmaceutically acceptable salts thereof, wherein A is a moiety of the formula: 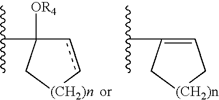
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.
Duloxetine

Duloxetine has the following structure:

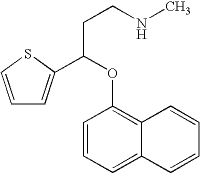

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno [2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO04/004734.

Other Compounds

In certain embodiments, the drug combinations described herein comprise one or more compounds selected from methotrexate, hydroxychloroquine, sulfasalazine, tacrolimus, sirolimus, mycophenolate mofetil, and methyl prednisolone.

Nonsteroidal Immunophilin-Dependent Immunosuppressants

In another embodiment, a drug combination comprises an antihistamine and a nonsteroidal immunophilin-dependent immunosupressant (NsIDI).

In one embodiment, the NsIDI is cyclosporine. In another embodiment, the NsIDI is tacrolimus. In another embodiment, the NsIDI is rapamycin. In another embodiment, the NsIDI is everolimus. In still other embodiments, the NsIDI is pimecrolimus or the NsIDI is a calcineurin-binding peptide. Two or more NsIDIs can be administered contemporaneously. Calcineurin inhibitors including cyclosporines, tacrolimus, pimecrolimus, and rapamycin are described in detail herein. In another embodiment, a drug combination comprises an antihistamine and a peptide moiety. Peptide moieties, including peptides, peptide mimetics, peptide fragments, either natural, synthetic or chemically modified, that impair the calcineurin-mediated dephosphorylation and nuclear translocation of NFAT that may be used in the drug combinations described herein are described in detail above.

In certain embodiments, the drug combination further comprising at least one other compound, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Other agents—either biologics or small molecules—that modulate an immune response may also be included in a drug combination. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNFα. Agents that inhibit TNFα include etanercept, adelimumab, infliximab, and CDP-870. Small molecule immunodulators include, for example, p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

In another embodiment, one or more agents typically used to treat COPD may be used as a substitute for or in addition to a corticosteroid in the drug combinations described herein. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, a drug combination features the combination of a tricyclic compound and a bronchodilator.

In another embodiment, one or more antipsoriatic agents typically used to treat psoriasis may be used as a substitute for or in addition to a corticosteroid in the drug combinations described herein. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, a drug combination features the combination of a tricyclic compound and an antipsoriatic agent.

In still another embodiment, one or more agents typically used to treat inflammatory bowel disease may be used as a substitute for or in addition to a corticosteroid in the drug combinations described herein. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, a drug combination features the combination of a tricyclic compound and any of the foregoing agents.

In still another embodiment, one or more agents typically used to treat rheumatoid arthritis may be used as a substitute for or in addition to a corticosteroid in the drug combinations described herein. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, a drug combination features the combination of a tricyclic compound with any of the foregoing agents.

In yet another embodiment, one or more agents typically used to treat asthma may be used as a substitute for or in addition to a corticosteroid in the drug combinations described herein. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, a drug combination features the combination of a tricyclic compound and any of the foregoing agents.

In one embodiment, a drug combination is provided that comprises an antihistamine or an antihistamine analog and a corticosteroid. In certain embodiments, the antihistamine is bromodiphenhydramine, clemizole, cyproheptadine, desloratadine, loratadine, thiethylperazine maleate, epinastine, or promethazine. In certain other embodiments, the corticosteroid is prednisolone, cortisone, dexamethasone, hydrocortisone, methylprednisolone, fluticasone, prednisone, triamcinolone, or diflorasone. In a particular embodiment, the antihistamine is desloratadine or loratadine and the corticosteroid is prednisolone. In other specific embodiments, the drug combination comprises prednisolone and any one of the anti-histamine compounds, bromodiphenhydramine, clemizole, cyproheptadine, thiethylperazine maleate, and promethazine.

In other certain embodiments, the drug combination comprises amoxapine (tricyclic compound) and any one of the antihistamine compounds bromodiphenhydramine, loratadine, cyproheptadine, desloratadine, clemizole, thiethylperazine maleate, and promethazine. In another embodiment, the drug combination comprises nortryptyline (tricyclic or tetracyclic antidepressant) and any one of the antihistamine compounds bromodiphenhydramine, loratadine, cyproheptadine, desloratadine, clemizole, thiethylperazine maleate, and promethazine. In another specific embodiment, the drug combination comprises paroxetine (an SSRI) and any one of the antihistamine compounds bromodiphenhydramine, loratadine, cyproheptadine, desloratadine, clemizole, thiethylperazine maleate, and promethazine. In still another specific embodiment, the drug combination comprises fluoxetine (an SSRI) and any one of the antihistamine compounds bromodiphenhydramine, loratadine, cyproheptadine, desloratadine, clemizole, thiethylperazine maleate, and promethazine. In one specific embodiment, the drug combination comprises setraline (an SSRI) and any one of the antihistamine compounds clemizole, desloratadine, and promethazine. In still another specific embodiment, the drug combination comprises despiramine and any one of the antihistamine compounds loratadine, clemizole, desloratadine, and promethazine.

In still other embodiments, prednisolone is combined with any one of the antihistamine compounds, azatidine, bromodiphenhydramine, cetrizine, chlorpheniramine, clemizole, cyproheptadine, desloratadine, dimenhydrinate, doxylamine, fexofenadine, loratadine, meclizine, promethazine, pyrilamine, thiethylperazine; and tripelennamine. In another specific embodiment, the drug combination comprises prednisolone and epinastine; in another specific embodiment, the drug combination comprises prednisolone and cyproheptadine.

In another embodiment, the drug combination comprises dipyridamole (a tetra substituted pyrimiodpyrimidine) and an anti-histamine, which is any one of bromodiphenhydramine, cyproheptadine, loratadine, and thiethylperazine.

In other embodiments, the drug combination may further comprise a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal immunophilin-dependent immunosuppressant, vitamin D analog, psoralen, retinoid, or 5-amino salicylic acid. In certain embodiments, the NSAID is ibuprofen, diclofenac, or naproxen. In other certain particular embodiments, the COX-2 inhibitor is rofecoxib, celecoxib, valdecoxib, or lumiracoxib. In another particular embodiment, the biologic is adelimumab, etanercept, or infliximab; and in another particular embodiment, the DMARD is methotrexate or leflunomide. In other particular embodiments, the xanthine is theophylline, and in other certain embodiments, the anticholinergic compound is ipratropium or tiotropium. In still another certain embodiment, the beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline. In another certain embodiment, the vitamin D analog is calcipotriene or calcipotriol; and in other certain embodiments, the psoralen is methoxsalen. In one certain embodiment, the retinoid is acitretin or tazoretene. In another specific embodiment, the 5-amino salicylic acid is mesalamine, sulfasalazine, balsalazide disodium, or olsalazine sodium. In still another specific embodiment, the small molecule immunomodulator is VX 702, SCIO 469, doramapimod, RO 30201195, SCIO 323, DPC 333, pranalcasan, mycophenolate, or merimepodib.

In another embodiment, a drug combination comprises an antihistamine or an antihistamine analog and ibudilast or an analog thereof. In a particular embodiment, the antihistamine is bromodiphenhydramine, clemizole, cyproheptadine, desloratadine, loratadine, thiethylperazine maleate, epinastine, or promethazine. In a specific embodiment, the drug combination comprises (i) desloratadine or loratadine and (ii) ibudilast. In another specific embodiment, the drug combination comprises bromodiphenhydramine and ibudilast; in another embodiment, the drug combination comprises cyproheptadine and ibudilast; and in still another embodiment, the drug combination comprises thiethylperazine maleate and idublast. In certain embodiments, the drug combination further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In one embodiment, the drug combination comprises an antihistamine or an antihistamine analog and rolipram or an analog thereof. In a particular embodiment, the antihistamine is bromodiphenhydramine, clemizole, cyproheptadine, desloratadine, loratadine, thiethylperazine maleate, epinastine, or promethazine. In a particular embodiment, the drug combination comprises desloratadine or loratadine and rolipram. In another specific embodiment, the drug combination comprises bromodiphenhydramine and rolipram; in another embodiment, the drug combination comprises cyproheptadine and rolipram; and in still another embodiment, the drug combination comprises thiethylperazine maleate and rolipram. In certain embodiments, the drug combination further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In another embodiment, the drug combination comprises an antihistamine or an antihistamine analog and a tetra-substituted pyrimidopyrimidine. In a certain embodiment, the antihistamine is bromodiphenhydramine, clemizole, cyproheptadine, desloratadine, loratadine, thiethylperazine maleate, epinastine, or promethazine. In a specific embodiment, the tetra-substituted pyrimidopyrimidine is dipyridimole. In another specific embodiment, the antihistamine is desloratadine or loratadine and the tetra-substituted pyrimidopyrimidine is dipyridimole. In another specific embodiment, the drug combination may further comprise an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In one embodiment, the drug combination comprises an antihistamine or an antihistamine analog and a tricyclic or tetracyclic antidepressant or analog thereof. In a particular embodiment, the antihistamine is bromodiphenhydramine, clemizole, cyproheptadine, desloratadine, loratadine, thiethylperazine maleate, epinastine, or promethazine. In another particular embodiment, the tricyclic antidepressant is nortryptiline, amoxapine, or desipramine. In one specific embodiment, the drug combination comprises clemizole and nortryptiline, and in another specific embodiment, the drug combination comprises clemizole and amoxapine. In another embodiment, the drug combination further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In still another embodiment, the drug combination comprises an antihistamine or an antihistamine analog and an SSRI or analog thereof. In certain embodiments, the antihistamine is bromodiphenhydramine, clemizole, cyproheptadine, desloratadine, loratadine, thiethylperazine maleate, epinastine, or promethazine. In other certain embodiments, the SSRI is paroxetine or fluoxetine. In another particular embodiment, the drug combination further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal immunophilin-dependent immunosuppressant, vitamin D analog, psoralen, retinoid, or 5-amino salicylic acid.

In yet another specific embodiment, the drug combination comprises desloratadine and cyclosporine, and in another specific embodiment, the drug combination comprises loratadine and cyclosporine.

Drug Combination Comprising a Triazole and an Aminopyridine

In certain embodiments, the drug combination that has anti-scarring activity comprises at least two agents, wherein at least one agent is a triazole compound and at least one second agent is an aminopyridine compound. In specific embodiments, the triazole is fluconazole or itraconazole and the aminopyridine is a diaminopyridine such as phenazopyridine (PZP).

Compounds useful in the invention include those described herein in any of their pharmaceutically acceptable forms, including isomers such as diastereomers and enantiomers, salts, solvates, and polymorphs thereof, as well as racemic mixtures of the compounds described herein.

Triazole Compounds

By “triazole” is meant any member of the class of anti-fungal compounds having a five-membered ring of two carbon atoms and three nitrogen atoms. A compound is considered “antifungal” if it inhibits growth of a species of fungus by at least 25%. Exemplary triazoles include, for example, fluconazole, terconazole, itraconazole, posaconazole (SCH 56592), ravuconazole (BMS 207147), and voriconazole (UK-109,496), the structures of which are depicted in the Table 1 below.

TABLE 1
Exemplary Triazole Compounds

| Name of | |
|---|---|
| Triazole | Structure |
| fluconazole | 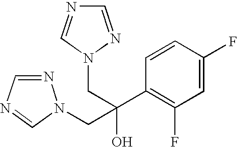 |
| itraconazole | 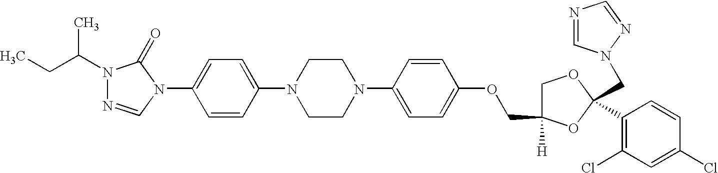 |
| terconazole | 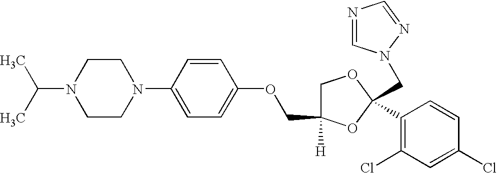 |
| posaconazole | 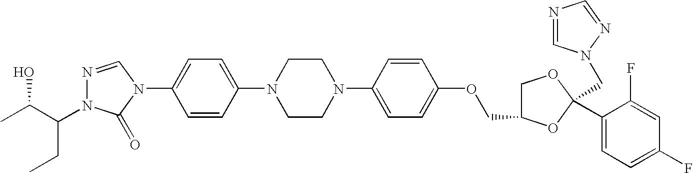 |
| ravuconazole | 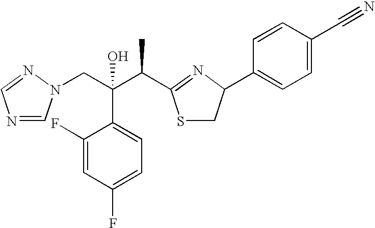 |
| voriconazole | 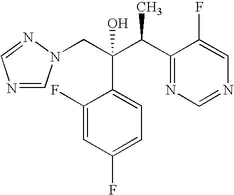 |

Aminopyridine Compounds

By “aminopyridine” is meant any pyridine ring-containing compound in which the pyridine has one, two, or three amino group substituents. Other substituents may optionally be present. Exemplary aminopyridines include, for example, phenazopyridine, 4-aminopyridine, 3,4-diaminopyridine, 2,5-diamino-4-methylpyridine, 2,3,6-triaminopyridine, 2,4,6-triaminopyridine, and 2,6-diaminopyridine, the structures of which are depicted in the Table 2 below.

TABLE 2
Exemplary Aminopyridine Compounds

| Aminopyridine Name | Structure |
|---|---|
| Phenazopyridine | 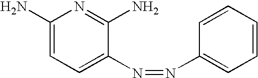 |
| 4-aminopyridine | 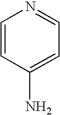 |
| 3,4-diaminopyridine | 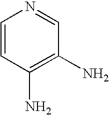 |
| 2,5-diamino-4-methylpyridine | 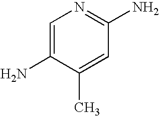 |
| 2,3,6-triaminopyridine | 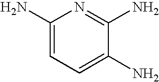 |
| 2,4,6-triaminopyridine | 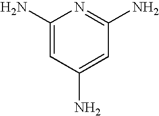 |
| 2,6-diaminopyridine | 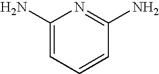 |

Compounds useful in the drug combination include those described herein in any of their pharmaceutically acceptable forms, including isomers such as diastereomers and enantiomers, salts, solvates, and polymorphs thereof, as well as racemic mixtures of the compounds described herein.

In certain embodiments, a drug combination comprises a triazole and an aminopyridine. In certain embodiments, the triazole is fluconazole, terconazole, itraconazole, voriconizole, posuconizole, or ravuconazole; in a certain specific embodiment, the triazole is fluconazole. In other certain embodiments, the aminopyridine is phenazopyridine, 4-amino-pyridine; 3,4-diaminopyridine; 2,5-diamino-4-methylpyridine; 2,3,6-triaminopyridine; 2,4,6-triaminopyridine; or 2,6-diaminopyridine; in a certain specific embodiment, the aminopyridine is phenazopyridine. In a specific embodiment, the triazole is fluconazole and the aminopyridine is phenazopyridine. In certain other embodiments, the triazole is itraconazole and the aminopyridine is phenazopyridine.

Drug Combination Comprising an Antiprotozoal Agent and an Aminopyridine and Drug Combination Comprising an Antiprotozoal Agent and a Quaternary Ammonium Compound

In certain embodiments, the drug combination that has anti-scarring activity comprises at least two agents, wherein at least one agent is an antiprotozoal agent and at least one second agent is an aminopyridine compound. In one specific embodiment, the antiprotozoal agent is pentamidine and the aminopyridine compound is a diaminopyridine such as phenazopyridine (PZP). In another embodiment, the drug combination that has anti-scarring activity comprises at least two agents, wherein at least one agent is an antiprotozoal agent and at least one second agent is a quaternary ammonium compound. In one specific embodiment, the antiprotozoal agent is pentamidine and the quaternary ammonium compound is pentolinium.

Antiprotozoal Agents

In one embodiment, an antiprotozoal agent is pentamidine or a pentamidine analog. Aromatic diamidino compounds can replace pentamidine in the antifungal combination of the invention. Aromatic diamidino compounds such as propamidine, butamidine, heptamidine, and nonamidine exhibit similar biological activities as pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy)phenamidine, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine.

In one embodiment, the antiprotozoal agent has the following structure having the formula (X): 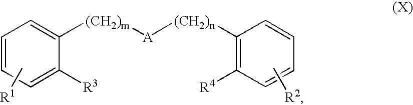
or a pharmaceutically acceptable salt thereof,
wherein A is 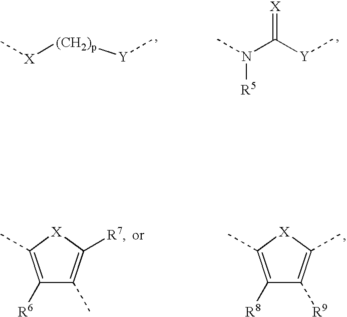
wherein each of X and Y is, independently, O, NR¹⁰, or S, each of R⁵ and R¹⁰ is, independently, H or C₁-C₆ alkyl, each of R⁶, R⁷, R⁸, and R⁹ is, independently, H, C₁-C₆ alkyl, halogen, C₁-C₆ alkyloxy,C₆-C₁₈ aryloxy, or C₆-C₁₈ aryl-C₁-C₆ alkyloxy, p is an integer between 2 and 6, inclusive, each of m and n is, independently, an integer between 0 and 2, inclusive, each of R¹ and R² is 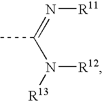
wherein R¹² is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 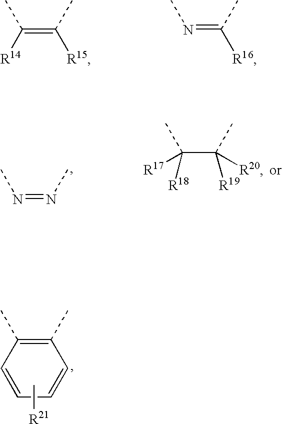
wherein each of R¹⁴, R¹⁵, and R₁₆ is, independently, H, C₁-C₆ alkyl, halogen, or trifluoromethyl, each of R¹⁷, R¹⁸, R¹⁹, and R²⁰ is, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, each of R³ and R⁴ is, independently, H, Cl, Br, OH, OCH₃, OCF₃, NO₂, and NH₂, or R³ and R⁴ together form a single bond.

In a related aspect, in the compound of formula (X), A is 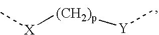
each of X and Y is independently O or NH, p is an integer between 2 and 6, inclusive, and m and n are, independently, integers between 0 and 2, inclusive, wherein the sum of m and n is greater than 0; or A is 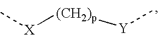
each of X and Y is independently O or NH, each of m and n is 0, and each of R¹ and R₂ is, independently, selected from the group represented by 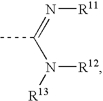
wherein R₁₂ is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl C₁-C₆ alkoxy), carbo(C₆-C₁₈ aryloxy), or C₆-C₁₈ aryl, and R¹¹ is H, OH, or C₁-C₆ alkyloxy, or R¹¹ and R₁₂ together represent 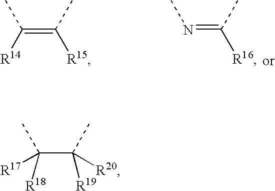
wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl; or A is 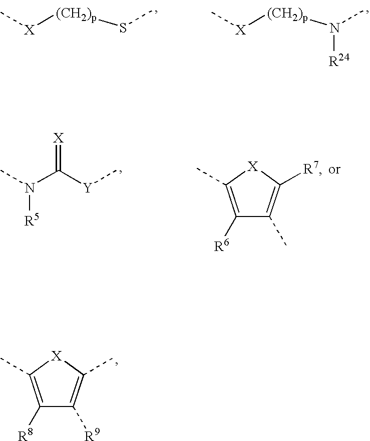
each of X and Y is, independently, O, NR¹⁰, or S, each of R⁵ and R¹⁰ is, independently, H or C₁-C₆ alkyl, each of R⁶, R⁷, R⁸, and R⁹ is, independently, H, C₁-C₆ alkyl, halogen, C₁-C₆ alkyloxy, C₆-C₁₈ aryloxy, or C₆-C₁₈ aryl C₁-C₆ alkyloxy, R²⁴ is C₁-C₆ alkyl, p is an integer between 2 and 6, inclusive, each of m and n is, independently, an integer between 0 and 2, inclusive, each of R¹ and R² is, independently, selected from the group represented by 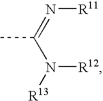
wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 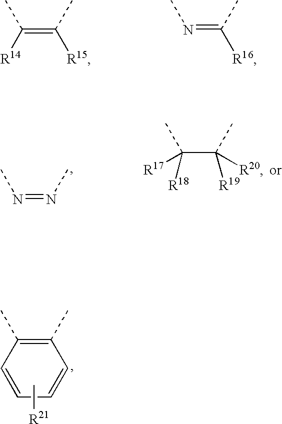
wherein each of R¹⁴, R¹⁵, and R¹⁶ is, independently, H, C₁-C₆ alkyl, halogen, or trifluoromethyl, each of R₁₇, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl.

Other analogs include stilbamidine (A-1) and hydroxystilbamidine (A-2), and their indole analogs (e.g., A-3).

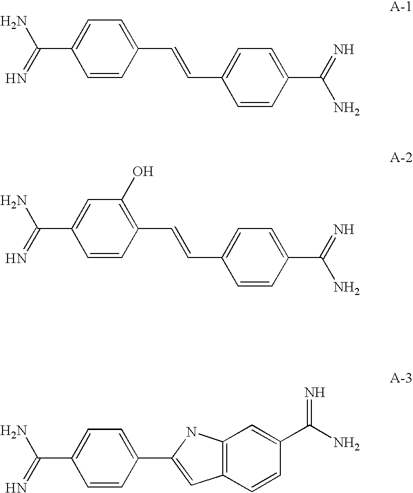

Each amidine moiety in A-1, A-2, or A-3 may be replaced with one of the moieties depicted in formula (X) above as

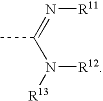

As is the case for pentamidine, salts of stilbamidine and its related compounds are also useful in the method of the invention. Preferred salts include, for example, dihydrochloride and methanesulfonate salts.

Still other analogs include the bis-benzamidoximes described in U.S. Pat. Nos. 5,723,495, 6,214,883, 6,025,398, and 5,843,980. Other diamidine analogs have also been described in U.S. Pat. Nos. 5,578,631, 5,428,051, 5,602,172, 5,521,189, 5,686,456, 5,622,955, 5,627,184, 5,606,058, 5,643,935, 5,792,782, 5,939,440, 5,639,755, 5,817,686, 5,972,969, 6,046,226, 6,156,779, 6,294,565, 5,817,687, 6,017,941, 6,172,104, and 6,326,395 each of which is herein incorporated by reference. Any of the amidine and diamidine analogs described in the foregoing patents can be used in a combination of the invention.

Exemplary analogs are 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2 -benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis [5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan. Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

Exemplary compounds having formula (X) include but are not limited to pentamidine, propamidine, butamidine, heptamidine, nonamidine, stilbamidine, hydroxystilbamidine, diminazene, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime. In specific embodiments, the compound of formula (X) is pentamidine, 2,5-bis(4-amidinophenyl)furan, or 2,5-bis(4-amidinophenyl)furan-bis-O-methylamidoxime.

As described herein a drug combination comprising an anti-protozoal agent may comprise an aromatic diamidine, which includes the following exemplary compounds: pentamidine, propamidine, butamidine, heptamidine, nonamidine, stilbamidine, hydroxystilbamidine, diminazene, benzamidine, phenamidine, dibrompropamidine, or any one of the pentamidine analogues described herein.

The structure of pentamidine is:

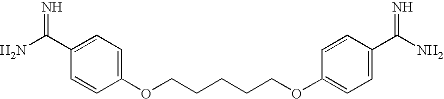

Pentamidine isethionate is a white, crystalline powder soluble in water and glycerin and insoluble in ether, acetone, and chloroform. Pentamidine is chemically designated 4,4′-diamidino-diphenoxypentane di(β-hydroxyethanesulfonate). The molecular formula is C₂₃H₃₆N₄O₁₀S₂ and the molecular weight is 592.68.

Recently, pentamidine was shown to be an effective inhibitor of protein tyrosine phosphatase 1B (PTP1B). Because PTP1B dephosphorylates and inactivates Jak kinases, which mediate signaling of cytokines with leishmanicidal activity, its inhibition by pentamidine might result in augmentation of cytokine signaling and anti-leishmania effects. Pentamidine has also been shown to be a potent inhibitor of the oncogenic phosphatases of regenerating liver (PRL). Pentamidine has also been shown to inhibit the activity of endo-exonuclease (PCT Publication No. WO 01/35935). Thus, in the methods of the invention, pentamidine can be replaced by any PTP1B inhibitor, PRL inhibitor, or endo-exonuclease inhibitor.

Pentamidine metabolites are also useful in the antifungal combination of the invention. Pentamidine is rapidly metabolized in the body to at least seven primary metabolites. Some of these metabolites share one or more activities with pentamidine. It is likely that some pentamidine metabolites will have antifungal activity when administered in combination with an antiproliferative agent. Seven pentamidine metabolites (B-1 through B-7) are shown below. 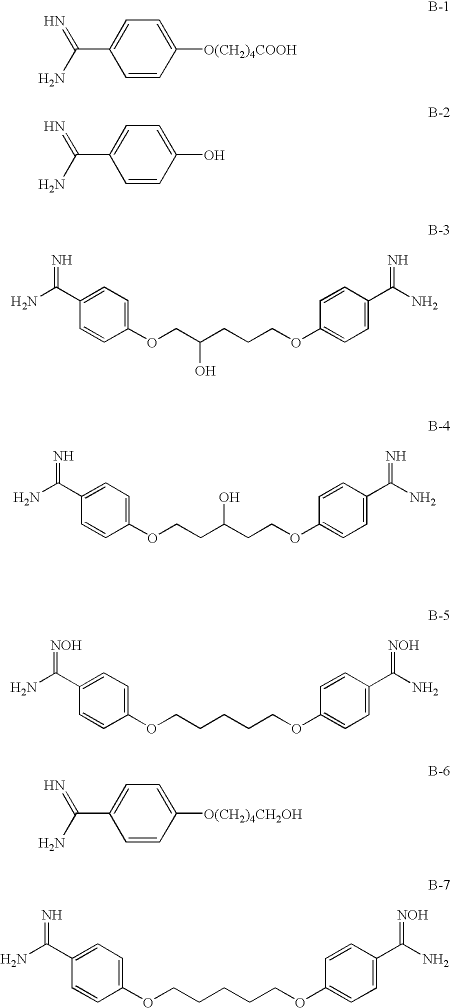
Aminopyridine Compounds

By “aminopyridine” is meant any pyridine ring-containing compound in which the pyridine has one, two, or three amino group substituents. Other substituents may optionally be present.

In one embodiment, the aminopyridine agent has a structure of the formula (XI): 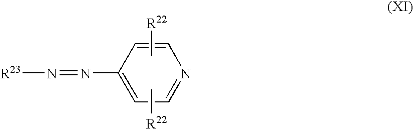
wherein each R₂₂ is, independently, NH₂, H, OH, a halide, C₁₋₁₀ alkyl, C₁₋₁₀ alkoxyalkyl, hydroxyalkyl (wherein the alkyl group has from 1 to 10 carbon atoms), aminoalkyl (wherein the alkyl group has from 1 to 10 carbon atoms), C₁₋₁₀ alkylaminoalkyl, cycloalkyl (wherein the alkyl group has from 1 to 10 carbon atoms), aryl, or C₁₋₁₀ alkylaryl; and R²³ is NH₂, H, OH, a halide, C₁₋₁₀ alkyl, C₁₋₁₀ alkoxyalkyl, hydroxyalkyl (wherein the alkyl group has from 1 to 10 carbon atoms), aminoalkyl (wherein the alkyl group has from 1 to 10 carbon atoms), C₁₋₁₀ alkylaminoalkyl, cycloalkyl (wherein the alkyl group has from 1 to 10 carbon atoms), aryl, or C₁₋₁₀ alkylaryl.

In one embodiment, the aminopyridine agent has the following structure having the compound having the formula (XII): 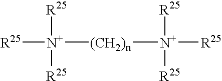
wherein each R²⁵ is, independently, NH₂, H, OH, a halide, C₁₋₁₀ alkyl, C₁₋₁₀ alkoxyalkyl, hydroxyalkyl (wherein the alkyl group has from 1 to 10 carbon atoms), aminoalkyl (wherein the alkyl group has from 1 to 10 carbon atoms), C₁₋₁₀ alkylaminoalkyl, cycloalkyl (wherein the alkyl group has from 1 to 10 carbon atoms), C₆₋₁₈ aryl, or C₁₋₁₀ alkylaryl; n is an integer between 2 and 10, inclusive.
Phenazopyridine

By “aminopyridine” is meant any pyridine ring-containing compound in which the pyridine has one, two, or three amino group substituents. Other substituents may optionally be present. Aminopyridines include phenazopyridine (C-1), 4-aminopyridine (C-2), 3,4-diaminopyridine (C-3), 2,5-diamino-4-methylpyridine (C-4), 2,3,6-triaminopyridine (C-5), 2,4,6-triaminopyridine (C-6), and 2,6-diaminopyridine (C-7), the structures of which are depicted below.

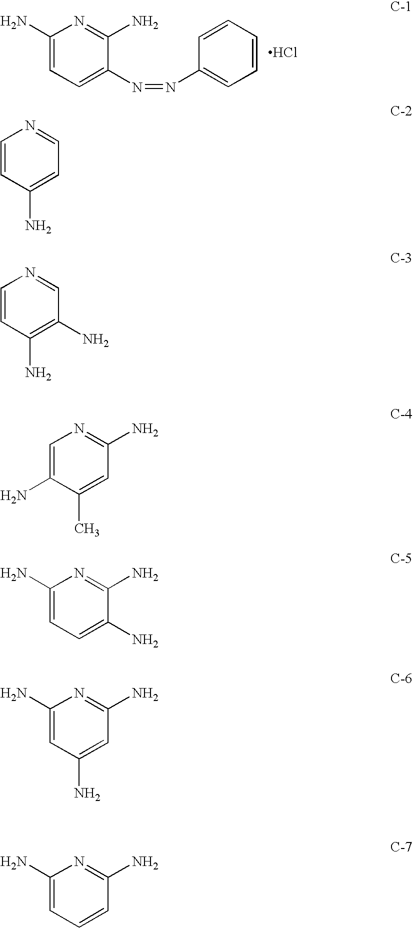

Aminopyridines can accommodate many modifications while still maintaining structural and therapeutic efficacy. Phenazopyridine and derivatives thereof have been disclosed in U.S. Pat. Nos. 1,680,108, 1,680,109, 1,680,110, and 1,680,111. Heterocyclic azo derivatives and N-substituted diaminopyridines have also been described (see, e.g., U.S. Pat. Nos. 2,145,579 and 3,647,808).

Aminopyridine compounds exhibit anti-fungal activity. Additional compounds that exhibit anti-fungal activity that may be included in the drug combination described herein include fluconazole, amphotericin B, nystatin, pimaricin, ketoconazole, miconazole, thiabendazole, emlkonazole, itraconazole, ravuconazole, posaconazole, voriconazole, dapsone, griseofulvin, carbol-fuchsin, clotrimazole, econazole, haloprogin, mafenide, naftifine, oxiconazole, silver sulfadiazine, sulconazole, terbinafine, amorolfine, tioconazole, tolnaftate, undecylenic acid, butoconazle, gentian violet, terconazole, flucytosine, ciclopirox, caspofungin acetate, micafungin, and V-echinocandin (LY303366).

Quaternary ammonium compounds

By “quaternary ammonium compound” is meant any quaternary ammonium-containing compound in which the nitrogen atom has four group substituents. Quaternary ammonium compounds may be mono-, symmetrical quaternary, or asymmetrical quaternary compounds.

Quaternary ammonium compounds include, for example, pentolinium (D-1), hexamethonium (D-2), pentamethonium (D-3), tetraethylammonium (D-4), tetramethylammonium (D-5), chlorisondamine (D-6), and trimethaphan (D-7), the structures of which are depicted below.

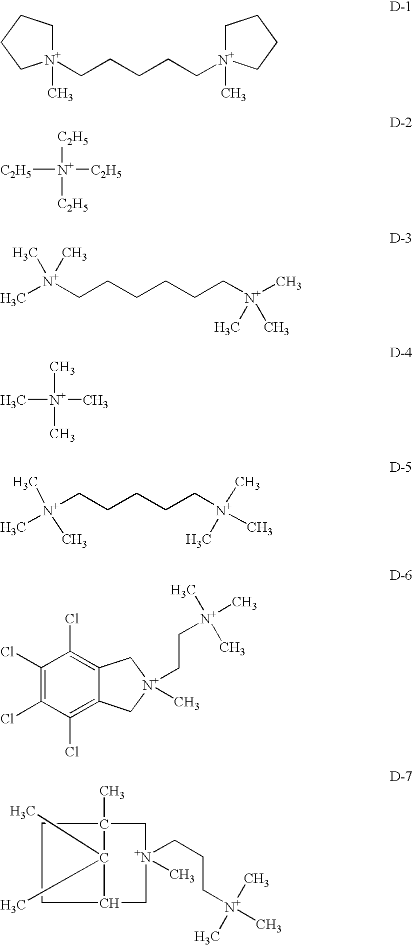

Pentolinium (pentamethylene-1,5-bis(N-methylpyrrolidinium) and its salt, pentolinium ditartrate, are symmetrical quaternary ammonium compounds. The tartrate salt form of pentolinium has the molecular formula C₂₃H₄₂N₂O₁₂ with a molecular weight of 538.6. Pentolinium ditartrate is a white powder, near odorless, and highly soluble in water.

Pentolinium Analogs

Quaternary ammonium compounds can accommodate many modifications while still maintaining structural and therapeutic efficacy. Pentolinium and its derivatives thereof are described in U.S. Pat. Nos. 4,902,720 and 6,096,788, each of which is herein incorporated by reference. Any of the quaternary ammonium compounds described in the foregoing patents can be used in a combination of the invention.

Compounds useful in the invention include those described herein in any of their pharmaceutically acceptable forms, including isomers such as diastereomers and enantiomers, salts, solvates, and polymorphs, thereof, as well as racemic mixtures of the compounds described herein.

In certain embodiments, the drug combination comprises (i) an aromatic diamidine or a compound having formula (X); and at least one of (ii) an aminopyridine; (iii) a quaternary ammonium compound; or (iv) a compound having one of formulas (XI) and (XII). In particular embodiments, aromatic diamidines suitable for use in the drug combinations described herein include pentamidine, propamidine, butamidine, heptamidine, nonamidine, stilbamidine, hydroxystilbamidine, diminazene, benzamidine, 4,4′-(pentamethylenedioxy)di-, dihydrochloride, phenamidine, dibrompropamidine, 1,3-bis(4-amidino-2-methoxyphenoxy)propane, netropsin, distamycin, and phenamidine. Aminopyridines suitable for use drug combinations described herein include phenazopyridine, 4-amino-pyridine, 3,4-diaminopyridine, 2,5-diamino-4-methylpyridine, 2,3,6-triaminopyridine, 2,4,6-triaminopyridine, and 2,6-diaminopyridine. Quaternary ammonium compounds suitable for the drug combinations described herein include pentolinium, hexamethonium, pentamethonium, tetramethylammonium, tetraethylammonium, trimethaphan, and chlorisondamine. In a specific embodiment, the drug combination comprises the aromatic diamidine pentamidine and phenazopyridine (aminopyridine). In another specific embodiment, the drug combination comprises pentamidine and the quaternary ammonium compound pentolinium.

In other embodiments, the drug combination may further comprise an antifungal agent wherein the antifungal agent is selected from amphotericin B, fluconazole, nystatin, pimaricin, ketoconazole, miconazole, thiabendazole, emlkonazole, itraconazole, ravuconazole, posaconazole, voriconazole, dapsone, griseofulvin, carbol-fuchsin, clotrimzole, econazole, haloprogin, mafenide, naftifine, oxiconazole, silver sulfadiazine, sulconazole, terbinafine, amorolfine, tioconazole, tolnaftate, undecylenic acid, butoconazle, gentian violet, terconazole, flucytosine, ciclopirox, caspofungin acetate, micafungin, and V-echinocandin (LY303366).

Drug Combination Comprising an Aromatic Diamidine and an Antiestrogen, Anti-fungal Imidazole, Disulfiram or Ribavirin

In certain embodiments, the drug combination that has anti-scarring activity comprises at least two agents, wherein at least one agent is an aromatic diamidine compound and at least one second agent is selected from an antiestrogen, an anti-fungal imidazole, disulfiram, and ribavirin. In a particular embodiment, an aromatic diamidine includes pentamidine, propamidine, butamidine, heptamidine, nonamidine, stilbamidine, hydroxystilbamidine, diminazene, benzamidine, 4,4′-(pentamethylenedioxy)di-, dihydrochloride, phenamidine, dibrompropamidine, 1,3-bis (4-amidino-2-methoxyphenoxy) propane, netropsin, distamycin, and phenamidine. In a specific embodiment, the aromatic diamidine is pentamidine. In other certain embodiments, an antiestrogen includes tamoxifen, 4-hydroxy tamoxifen, clomifene, raloxifene, and faslodex. In a specific embodiment, the antiestrogen is tamoxifen. In another particular embodiment, an anti-fungal imidazole compound includes ketoconazole, sulconazole, clotrimazole, econazole, miconazole, oxiconazole, tioconazole, and butoconazole. In a specific embodiment, the anti-fungal imidazole compound is ketoconazole or sulconazole. In certain specific embodiments, the drug combination comprises pentamidine and disulfiram; in another specific embodiment, the drug combination comprises pentamidine and ketoconazole; in still another specific embodiment, the drug combination comprises pentamidine and ribavirin; in yet another specific embodiment, the drug combination comprises pentamidine and sulconazole; and in still another specific embodiment, the drug combination comprises pentamidine and tamoxifen.

Aromatic diamidine compounds are described in detail herein and any one of these described compounds may be included in the drug combinations described herein. Particularly, pentamidine, pentamidine analogs, aromatic diamidine compounds comprising a structure having the formula (X); pentamidine metabolites (B-1 through B-7) are described. Other analogs include stilbamidine (A-1) and hydroxystilbamidine (A-2), and their indole analogs (e.g., A-3) and are also described in detail herein. Exemplary compounds having a structure of formula (X) and exemplary compounds that are pentamidine analogs are also provided herein.

Pentamidine Analogs

In addition, to the pentamidine analogs described above, pentamidine analogs include the following. Aromatic diamidino compounds can replace pentamidine in the antiproliferative combinations of the invention. Aromatic diamidines such as propamidine, butamidine, heptamidine, and nonamidine share properties with pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy) di-, dihydrochloride, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine.

Certain pentamidine analogs are described, for example, by formula (XIII). 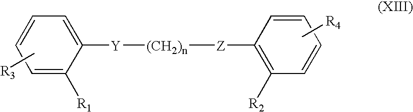
wherein each of Y and Z is, independently, O or N; each of R₁ and R₂ is, independently, NH₂, H, OH, a halide, C₁₋₅ alkyl, C₁₋₅ alkoxyalkyl, hydroxyalkyl (wherein the alkyl group has from 1 to 5 carbon atoms), aminoalkyl (wherein the alkyl group has from 1 to 5 carbon atoms), C₁₋₅ alkylaminoalkyl, cycloalkyl (wherein the alkyl group has from 1 to 5 carbon atoms), aryl, or C₁₋₅ alkylaryl; and n is an integer from 2 to 6, inclusive; and each of R₃ and R₄ is, independently, at the meta- or para- position and is selected from the group consisting of: 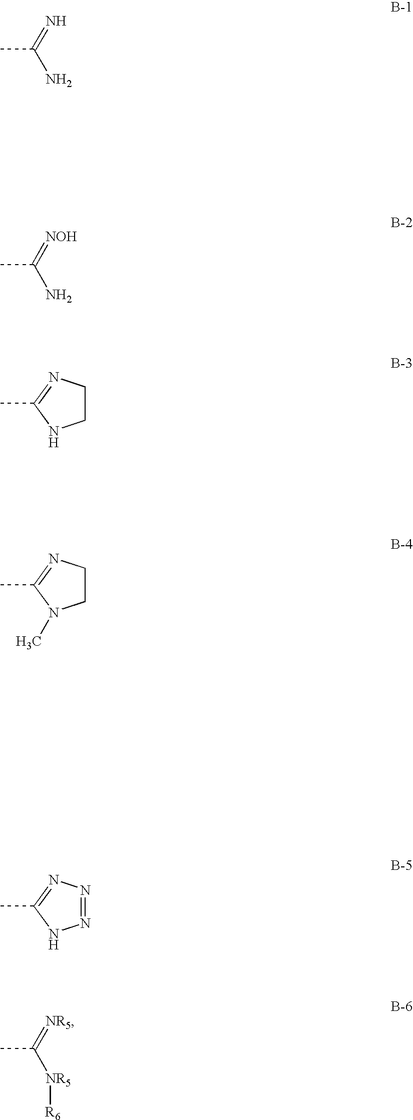
wherein each of R₅ and R₆ is, independently, NH₂, H, OH, a halide, C₁₋₅ alkyl, C₁₋₅ alkoxyalkyl, hydroxyalkyl (wherein the alkyl group has from 1 to 5 carbon atoms), aminoalkyl (wherein the alkyl group has from 1 to 5 carbon atoms), C₁₋₅ alkylaminoalkyl, cycloalkyl (wherein the alkyl group has from 1 to 5 carbon atoms), aryl, or C₁₋₅ alkylaryl.
Anti-Estrogenic Compounds

By “antiestrogen” or “antiestrogenic compound” is meant any agent that blocks an activity of estrogen. These agents may act to competitively or non-competitively inhibit the binding of estrogen to one of its receptors. Certain antiestrogens selectively bind to an estrogen receptor and inhibit the binding of estrogen to the receptor. Binding of the antiestrogens to the ERs may induce structural change in the engaged ER to inhibit DNA binding, dimerization, protein-protein interactions, or ER nuclear localization.

Exemplary antiestrogenic compounds are tamoxifen (K-1), 4-hydroxy tamoxifen (K-4), clomifene (K-2), raloxifene (K-5), and faslodex (ICI 182,780; K-3), the structures of which, are depicted below.

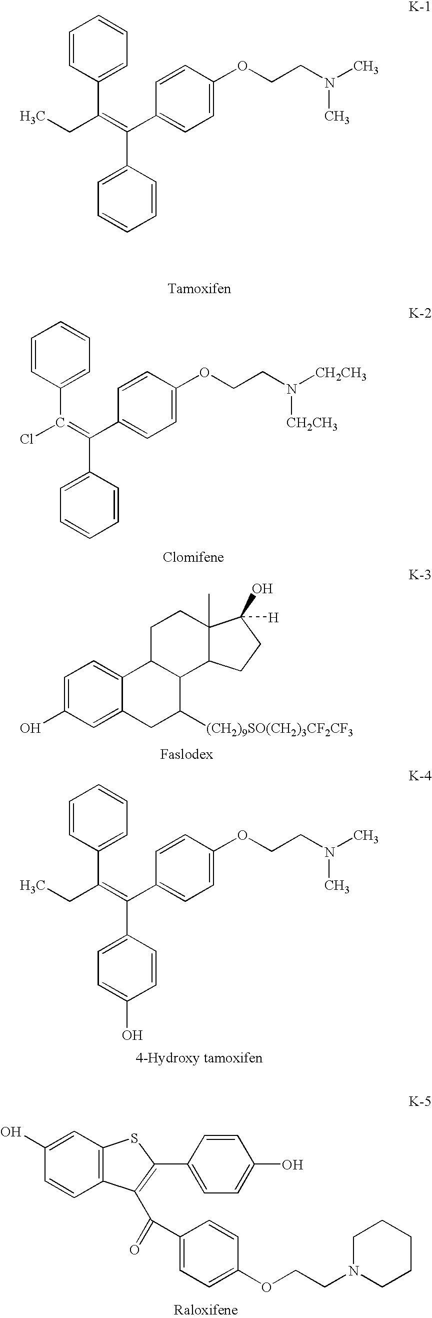

Tamoxifen is a non-steroidal estrogen antagonist, used alone or as an adjunct to surgery and/or radiation therapy for the treatment of breast cancer. Tamoxifen is prepared as a citrate salt for oral administration. Tamoxifen citrate is a fine, white crystalline powder, with a solubility of 0.5 mg/mL in water and a pKₐ of 8.85. Tamoxifen metabolites include N-desmethyltamoxifen and 4-hydroxy tamoxifen is also observed.

Antifungal Imidazoles

One biological activity of the imidazole family of antifungal agents works is inhibition of cytochrome P450 14-α-demethylase in fungal cells. This enzyme is involved in the conversion of lanosterol to ergosterol, which is the major sterol found in fungal cell membranes. The structures of suitable imidazole antifungal compounds are presented below.

Ketoconazole and sulconazole are two synthetic antifungal imidazoles. Ketoconazole is a white to slightly beige powder and is essentially insoluble in water. Ketoconazole has pKₐs of 2.9 and 6.5.

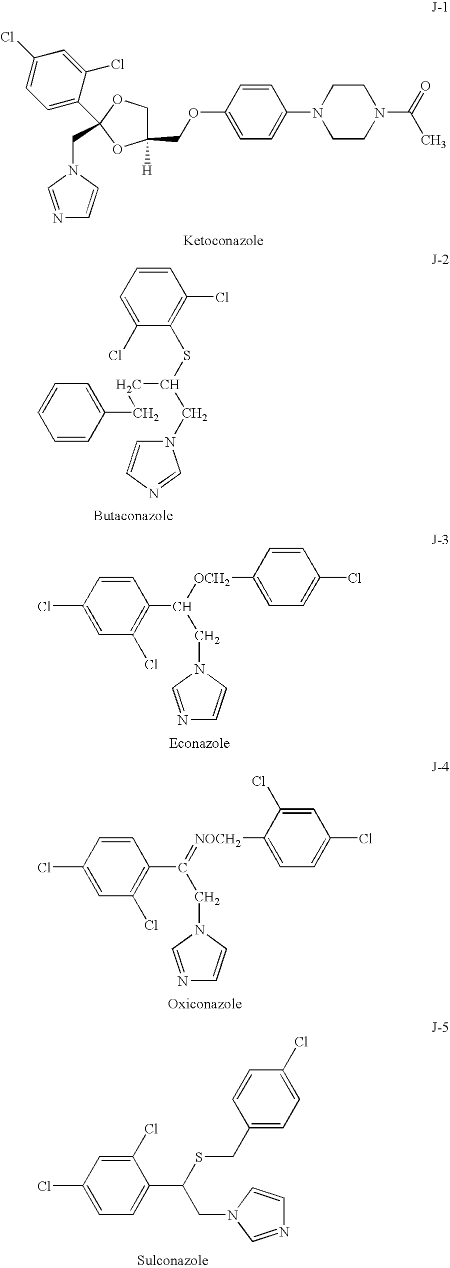

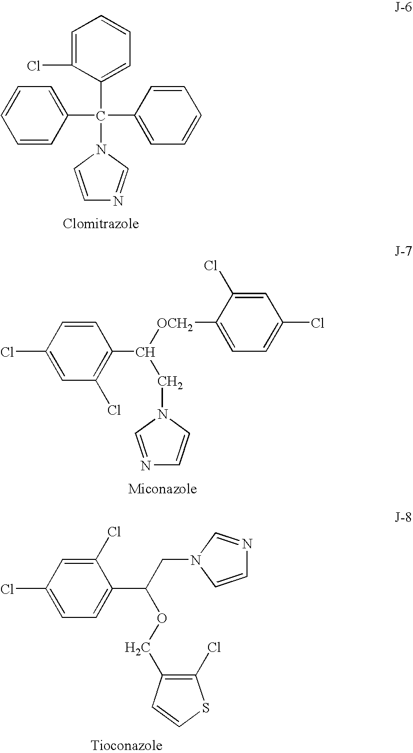

Disulfiram, more commonly known as Antabuse®, is commonly used in the treatment of alcoholism. This drug inhibits the enzyme-mediated step of acetaldehyde metabolism to acetate during alcohol catabolism.

Ribavirin is a synthetic nucleoside analog resembling guanosine. This drug is used as an anti-viral agent, blocking nucleotide synthesis and subsequently viral replication. Ribavirin inhibits both RNA and DNA virus replication. Ribavirin may be obtained as a white crystalline powder that is both odorless and tasteless. This drug is soluble in water (142 mg/mL), but only slightly soluble in alcohol.

Drug Combination Comprising an Aminopyridine and a Phenothiazine, Dacarbazine or Phenelzine

In certain embodiments, the drug combination that has anti-scarring activity comprises at least two agents, wherein at least one agent is an aminopyridine and at least one second agent is selected from a phenothiazine compound, dacarbazine, and phenelzine. In certain specific embodiments, aminopyridines include phenazopyridine, 4-amino-pyridine, 3,4-diaminopyridine, 2,5-diamino-4-methylpyridine, 2,3,6-triaminopyridine, 2,4,6-triaminopyridine, and 2,6-diaminopyridine. In a particular embodiment, the aminopyridine is phenazopyridine. In certain specific embodiments, phenothiazines include perphenazine, chlorpromazine, prochlorperazine, mepazine, methotrimeprazine, acepromazine, thiopropazate, perazine, propiomazine, putaperazine, thiethylperazine, methopromazine, chlorfenethazine, cyamemazine, enanthate, trifluoperazine, thioridazine, and norchlorpromazine. In a particular embodiment, the phenothiazine is perphenazine. In a particular embodiment, the drug combination comprises phenazopyridine and dacarbazine. In another particular embodiment, the drug combination comprises phenazopyridine and perphenazine. In another specific embodiment, the drug combination comprises phenazopyridine and phenelzine.

Aminopyridine Compounds

By “aminopyridine” is meant any pyridine ring-containing compound in which the pyridine has one, two, or three amino group substituents. Other substituents may optionally be present. Exemplary aminopyridines include, for example, phenazopyridine, 4-aminopyridine, 3,4-diaminopyridine, 2,5-diamino-4-methylpyridine, 2,3,6-triaminopyridine, 2,4,6-triaminopyridine, and 2,6-diaminopyridine, the structures of which are depicted in the table entitled “Exemplary Aminopyridine Compounds” herein.

Phenazopyridine

Phenazopyridine (PZP) is an exemplary aminopyridine. Other aminopyridines similar to phenazopyridine include 4-aminopyridine (E-1), 3,4-diaminopyridine (E-4), 2,5-diamino-4-methylpyridine (E-2), 2,3,6-triaminopyridine (E-5), 2,4,6-triaminopyridine (E-3), and 2,6-diaminopyridine (E-6), the structures of which are depicted below.

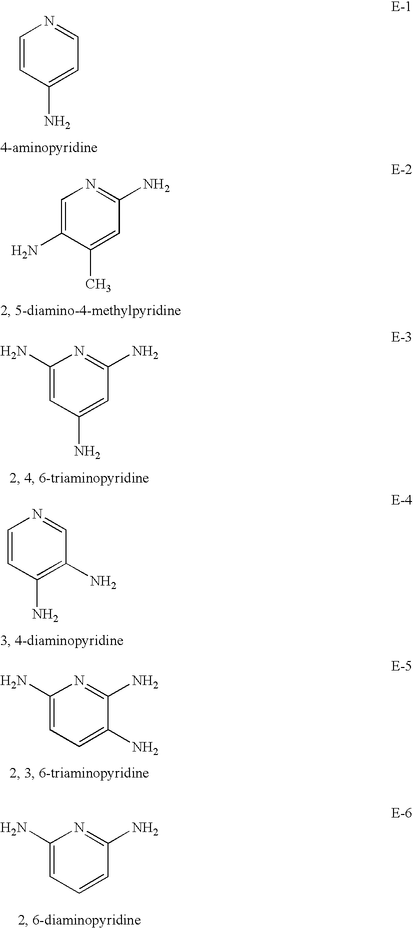

Phenazopyridine base (2,6-diamino-3-(phenylazo)pyridine) and its salt, phenazopyridine-HCl, are classified as medicinal azo dyes. The HCl salt form of phenazopyridine has the molecular formula C₁₁H₁₂ClN₅ with a molecular weight of 249.7. They are light to dark red to dark violet crystalline powders, near odorless, and slightly soluble in water and alcohol. Pharmaceutical phenazopyridine is usually synthesized as an HCl salt and prepared in tablet form. Phenazopyridine is usually prescribed to treat dysuria and urinary tract infections (UTI), acting as a local analgesic, and is not in itself a xenobiotic. Phenazopyridine is often prescribed in combination with sulphonamide compounds for treating UTIs. The structure of phenazopyridine -HCl is: 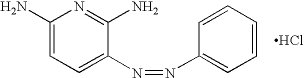
Phenazopyridine and aminopyridine analogs

Aminopyridines can accommodate many modifications while still maintaining structural and therapeutic efficacy. Phenazopyridine and derivatives thereof have been disclosed in U.S. Pat. Nos. 1,680,108; 1,680,109; 1,680,110; and 1,680,111. Modification of the medicinal azo dyes, di-amino(phenylazo)pyridines have been performed to improve solubility in water by reacting these compounds with alkylating agents (e.g., alkyl halides and alkyl sulphates) to produce quaternary pyridinium bases (see, e.g., U.S. Pat. No. 2,135,293). Heterocyclic azo derivatives and N-substituted diaminopyridines have also been described (U.S. Pat. Nos. 2,145,579 and 3,647,808, hereby incorporated by reference).

Phenazopyridine Metabolites

Phenazopyridine metabolites have been previously described in the literature (e.g., Thomas et al., J. Pharm. Sci. 79:321-325, 1990 and Jurima-Romet et al., Biopharm. Drug Disp. 14:171-179, 1992; hereby incorporated by reference). In humans, the major urinary phenazopyridine metabolite is the hydroxylation product of the pyridine ring, 2,6-diamino-5-hydroxy-3-(phenylazo)pyridine (5-OH- phenazopyridine). Other minor hydroxylated phenazopyridine metabolites include 2,6-diamino-5,4′-dihydroxy-3-(phenylazo)pyridine, 2,6-diamino-4′-hydroxy-3-(phenylazo)pyridine, and 2,6-diamino-2′-hydroxy-3-(phenylazo)pyridine. Cleavage of the azo bond results in the formation of a tri-aminopyridine and an aniline. The tri-aminopyridine metabolites can subsequently be further metabolized to mono, di, or other tri-aminopyridines and the aniline to aminophenols respectively.

Phenothiazines

Phenothiazines that are useful in the antimicrobial combination of the invention are compounds having the general formula (XIV): 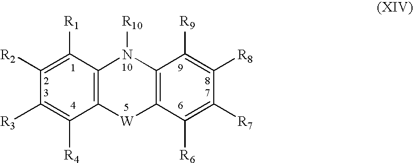
wherein R₂ is selected from the group consisting of: 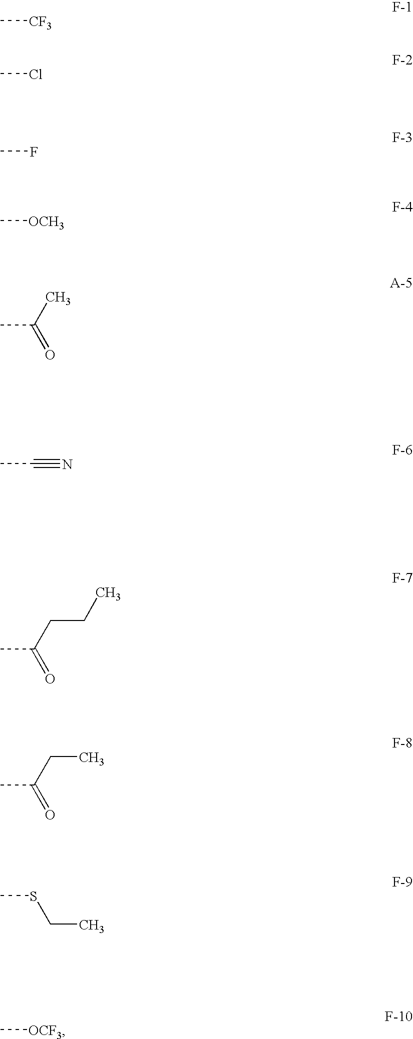
wherein each of R₁, R₃, R₄, R₅, R₆, R₇, R₈, and R₉ is, independently, H, OH, F, OCF₃,or OCH₃; and wherein W is selected from the group consisting of: 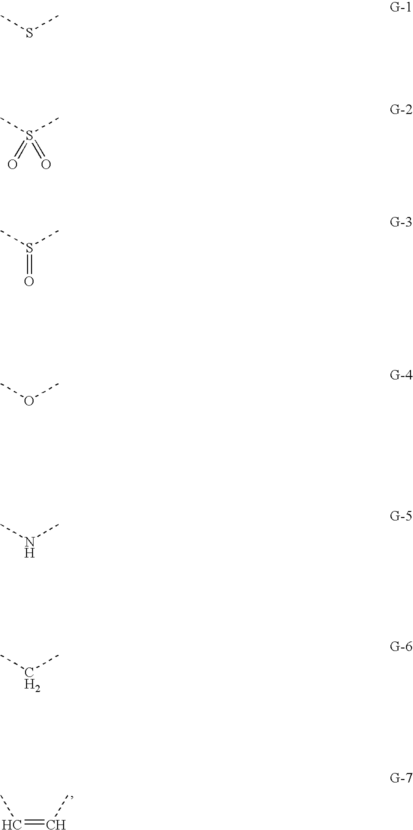
wherein R₁₀ is selected from the group consisting of:

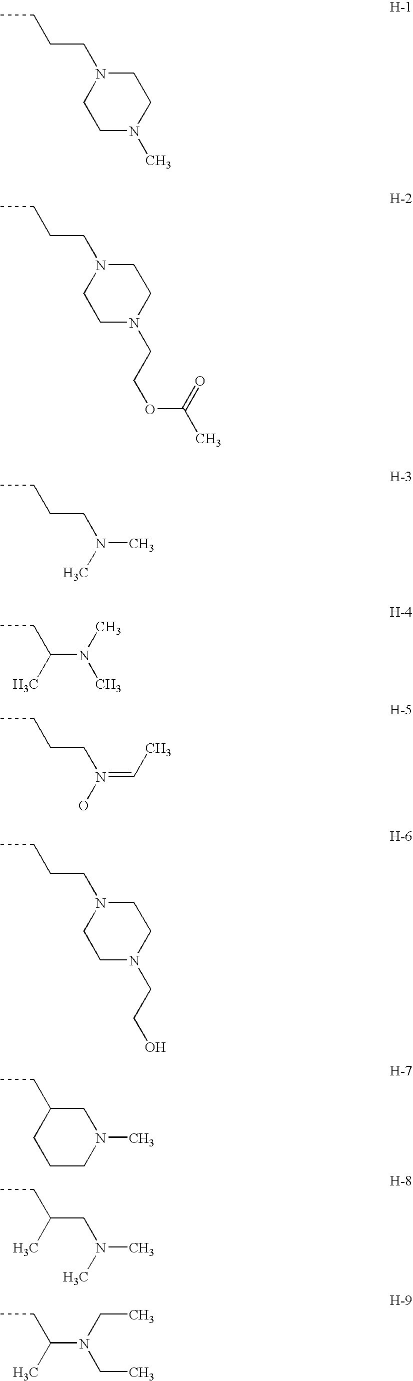

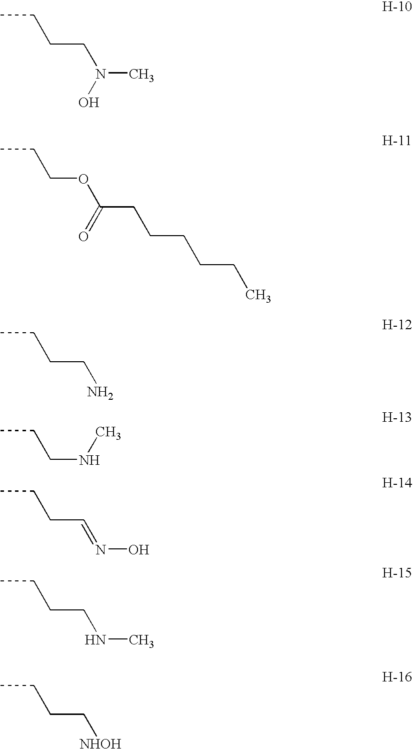

In certain embodiments of the compounds, R₂ is Cl; each of R₁, R₃, R₄, R₅, R₆, R₇, R₈, and R₉ is H or F. In other certain embodiments, each of R₁, R₄, R₅, R_{6,} and R₉ is H.

A commonly prescribed member of the phenothiazine family is perphenazine, which has the following formula:

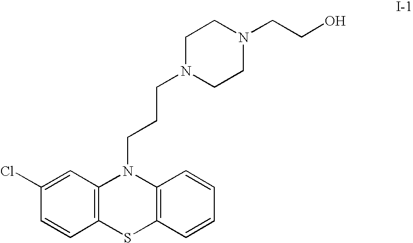

Perphenazine is currently formulated for oral and systemic administration. Perphenazine is a white-light yellow crystal or crystalline powder and is easily soluble in methanol, ethanol, and chloroform. It is slightly soluble in ether and shows relative insolubility in water. It is chemically designated 4-[3-(2-chlorophenothiazin-10-yl)propyl]-1-piperazineethanol and has a molecular formula of C21H 26ClN3OS with a molecular weight of 403.97.

Phenothiazines undergo extensive metabolic transformation into a number of metabolites that may be therapeutically active. These metabolites may be substituted for phenothiazines in the antimicrobial combinations of the invention. The metabolism of perphenazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide.

Dacarbazine, an antineoplastic agent, is a synthetic analog of a purine precursor and is used for the treatment of metastatic melanoma and Hodgkin's lymphoma. Dacarbazine is colorless to ivory colored crystalline and is poorly soluble in water and ethanol. Dacarbazine is poorly absorbed from the GI tract and is most commonly administered as an i.v. injection or infusion. Following i.v. injection, dacarbazine is metabolized, mostly in the liver, to its active form, as a monomethyl triazino derivative—the same active metabolite seen in an analog of dacarbazine, temozolomide.

Phenelzine, a hydrazine, is a yellowish-white powder that is highly soluble in water and very poorly soluble in alcohol.

Drug Combination Comprising a Quaternary Ammonium Compound and an Anti-fungal Imidazole, Haloprogin, Manganese Sulfate or Zinc Chloride

In certain embodiments, the drug combination that has anti-scarring activity comprises at least two agents, wherein at least one agent is a quaternary ammonium compound and at least one second agent is selected from an an anti-fungal imidazole, haloprogin, manganese sulfate (MnSO₄) and zinc chloride (ZnCl₂). In a particular embodiment, the quaternary ammonium compound includes pentolinium, hexamethonium, pentamethonium, tetramethylammonium, tetraethylammonium, trimethaphan, trimethidium, and chlorisondamine. In a particular embodiment, the quaternary ammonium compound is pentolinium. In another particular embodiment, an anti-fungal imidazole compound includes ketoconazole, sulconazole, clotrimazole, econazole, miconazole, oxiconazole, tioconazole, and butoconazole. In a specific embodiment, the anti-fungal imidazole compound is ketoconazole or sulconazole. In a specific embodiment, the drug combination comprises pentolinium and haloprogin; in another specific embodiment, the drug combination comprises pentolinium and manganese sulfate; in yet another specific embodiment, the drug combination comprises pentolinium and zinc chloride; and in another specific embodiment, the drug combination comprises pentolinium and sulconazole.

Quaternary Ammonium Compounds

Quaternary ammonium compounds are those in which the nitrogen atom has four group substituents. Quaternary ammonium compounds may be mono-, symmetrical bisquaternary, or asymmetrical bisquaternary compounds. Exemplary quaternary ammonium compounds are pentolinium (L-1), hexamethonium (L-3), pentamethonium (L-5), tetramethylammonium (L-4), tetraethylammonium (L-2), trimethidium (L-7), and chlorisondamine (L-6), the structures of which are depicted below.

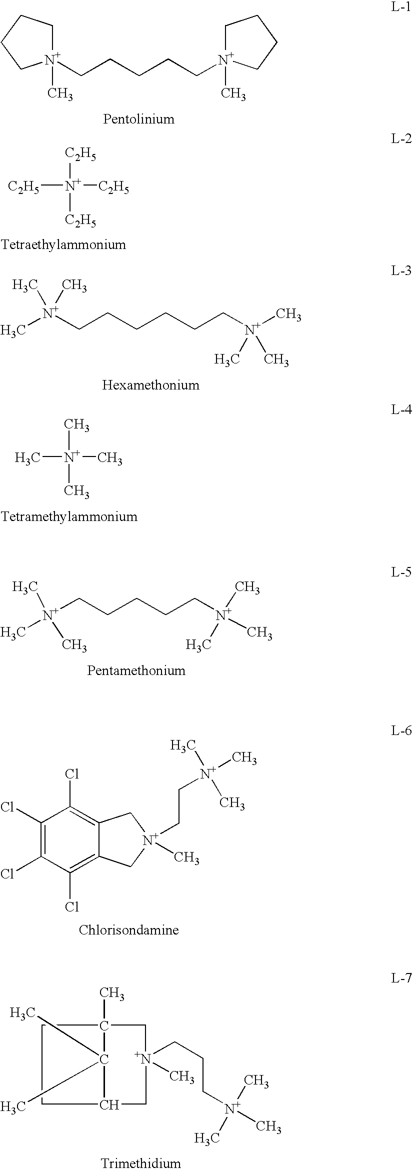

Pentolinium (pentamethylene-1,5-bis(N-methylpyrrolidinium) and its salt, pentolinium ditartrate, are symmetrical bisquaternary ammonium compounds. The tartrate salt form of pentolinium has the molecular formula C₂₃H₄₂N₂O₁₂ with a molecular weight of 538.6. Pentolinium ditartrate is a white powder, near odorless, and highly soluble in water.

The aforementioned quaternary ammonium compounds exhibit peripheral ganglionic blocking activity and have been used in anesthesia for controlled hypotension. The structure of pentolinium ditartrate (M-1) is: 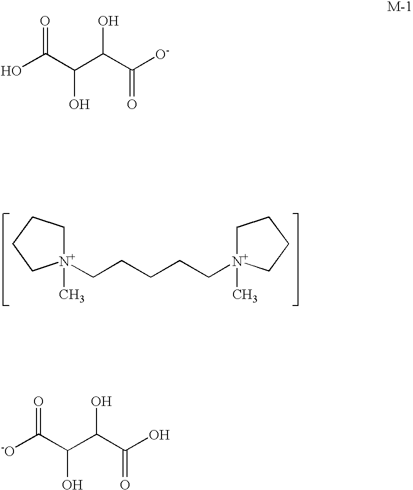
Pentolinium Analogs

Quaternary ammonium compounds can accommodate many modifications while still maintaining structural and therapeutic efficacy. Pentolinium and its derivatives are described in U.S. Pat. Nos. 4,902,720 and 6,096,788, each of which is hereby incorporated by reference. Any of the quaternary ammonium analogs described in these patents can be used in a drug combination described herein.

Haloprogin is a halogenated phenolic ether having the chemical formula C₉H₄C₁₃IO. This drug is used in the treatment of surface fungal infections, for example, tinea pedis (athlete's foot), tinea cruris, tinea corporis, and tinea manuum.

Drug Combination Comprising an Antiestrogen and a Phenothiazine, Cupric Chloride, Dacarbazine, Methoxsalen, or Phenelzine

In certain embodiments, the drug combination that has anti-scarring activity comprises at least two agents, wherein at least one agent is an antiestrogen compound and at least one second agent is selected from phenothiazine, cupric chloride, dacarbazine, methoxsalen, and phenelzine. In specific embodiments, antiestrogens include tamoxifen, 4-hydroxy tamoxifen, clomifene, raloxifene, and faslodex. In certain specific embodiments, the antiestrogen is tamoxifen. In certain embodiments, a phenothiazine is selected from perphenazine, chlorpromazine, prochlorperazine, mepazine, methotrimeprazine, acepromazine, thiopropazate, perazine, propiomazine, putaperazine, thiethylperazine, methopromazine, chlorfenethazine, cyamemazine, enanthate, trifluoperazine, thioridazine, and norchlorpromazine. In a particular embodiment, the phenothiazine is perphenazine. In a specific embodiment, the drug combination comprises tamoxifen and cupric chloride; in another specific embodiment, the drug combination comprises tamoxifen and dacarbazine; in still another specific embodiment, the drug combination comprises tamoxifen and methoxsalen; in another specific embodiment, the drug combination comprises tamoxifen and perphenazine; and in still another specific embodiment, the drug combination comprises tamoxifen and phenelzine.

As described herein exemplary antiestrogenic compounds are tamoxifen (K-1), 4-hydroxy tamoxifen (K-4), clomifene (K-2), raloxifene (K-5), and faslodex (ICI 182,780; K-3), the structures of which, are depicted above. Phenothiazines, for example, compounds having the structure of formula (XIV), derivatives, and metabolites thereof are described in greater detail herein. Dacarbazine as described herein exhibits antineoplastic activity and is used for the treatment of metastatic melanoma and Hodgkin's lymphoma. Dacarbazine is colorless to ivory colored crystalline and is poorly soluble in water and ethanol. Following intravenous injection, dacarbazine is metabolized, mostly in the liver, to its active form, as a monomethyl triazino derivative—the same active metabolite seen in an analog of dacarbazine, temozolomide.

Methoxsalen is a white to cream colored, odorless crystal, which is very poorly soluble in water, slightly soluble in alcohol, and readily soluble in propylene glycol. This drug is well absorbed in the GI tract and is available as a composition that may be used in oral and topical forms. Methoxsalen is rapidly demethylated to 8-hydroxypsoralen and can subsequently conjugated with glucuronic acid and sulphate.

Certain compounds used in the drug combinations described herein include disulfiram, methoxsalen, phenelzine, ribavirin, estradiol, dacarbazine, haloprogin, and temozolomide, the structures of which are illustrated below. All of the compounds described here are each separately known in the art; see e.g., Goodman & Gilman's The Pharmacological Basis of Therapeutics, Tenth Edition (J. G. Hardman, L. E. Limbird, A. G. Gilman, eds.), McGraw-Hill, New York, 2001; and hereby incorporated by reference. 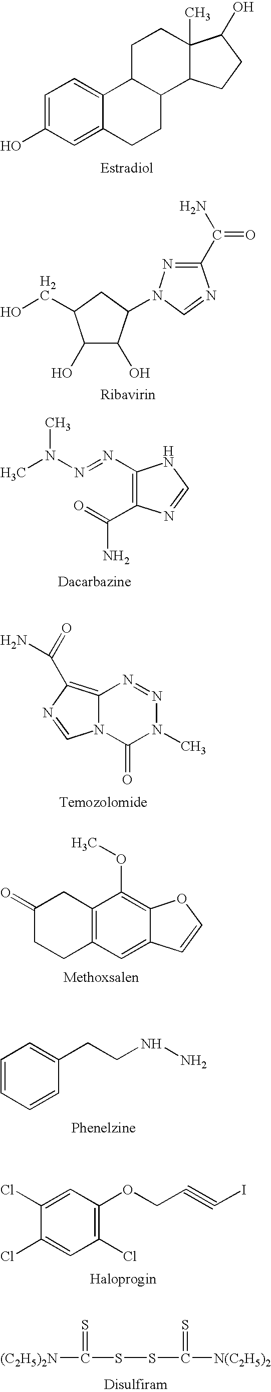
Drug Combination Comprising an Antifungal Imidazole and Disulfiram or Ribavirin

In certain embodiments, the drug combination that has anti-scarring activity comprises at least two agents, wherein at least one agent is an antifungal imidazole compound and at least one second agent is either disulfiram or ribavirin. In certain specific embodiments, anti-fungal imidazole compounds include ketoconazole, sulconazole, clotrimazole, econazole, miconazole, oxiconazole, tioconazole, and butoconazole. In a particular embodiment, the anti-fungal imidazole compound is ketoconazole or sulconazole. Each of the compounds in this drug combination have been described in detail herein. In a specific embodiment, the drug combination comprises ketoconazole and disulfiram; in another specific embodiment, the drug combination comprises ketoconazole and ribavirin.

Drug Combination Comprising an Estrogen and Dacarbazine

In certain embodiments, the drug combination that has anti-scarring activity comprises at least two agents, wherein at least one agent is an estrogen compound and at least one second agent is dacarbazine. In specific embodiments, estrogenic compounds include estradiol, estradiol valerate, estradiol cypionate, ethinyl estradiol, estriol, mestranol, quinestrol, estrone, estrone sulfate, equilin, diethylstilbestrol, and genistein. In a particular embodiment, the estrogenic compound is estradiol, or a salt of estradiol. In a specific embodiment, the drug combination comprises estradiol and dacarbazine. Dacarbazine is described herein.

As used herein, an “estrogenic compound” means any compound that has an activity of estrogen. These activities include binding to the estrogen receptors ERα and ERβ, and promoting the effects of such binding, including DNA-binding, dimerization, and transcriptional activation of target genes. Estrogenic compounds can be naturally-occurring (e.g., estradiol, estron, and estriol) or synthetic, non-steroidal compounds (e.g., diethylstilbesterol and genistein). Dacarbazine is described herein.

As described herein compounds useful in the drug combinations include those described herein in any of their pharmaceutically acceptable forms, including isomers such as diastereomers and enantiomers, salts, solvates, and polymorphs thereof, as well as racemic mixtures and pure isomers of the compounds described herein.

Drug Combination Comprising an Amphotericin Compound and a Dithiocarbamoyl Disulfide Compound

In certain embodiments, the drug combination that has anti-scarring activity comprises at least two agents, wherein at least one agent is an antifungal drug, such as an amphotericin, particularly amphotericin B, and at least one second agent is a dithiocarbamoyl disulfide compound, such as disulfiram. On the basis of similar activity among different antifungal agents, amphotericin can be replaced by a different antifungal agent in the combination. Likewise, on the basis of similar activity among different dithiocarbamoyl disulfide family members, disulfiram can be replaced by a different dithiocarbamoyl disulfide in the combination.

In certain specific embodiments, the antifungal agent is chosen from amphotericin B, amorolfine, anidulafungin, butenafine, butoconazole, candidin, carbol-fuchsin, caspofungin, ciclopirox, clotrimazole, dapsone, econazole, enilconazole, fluconazole, flucytosine, gentian violet, griseofulvin, haloprogin, itraconazole, ketoconazole, mafenide, micafungin, miconazole, naftifine, nystatin, oxiconazole, pimaricin, posaconazole, ravoconazole, rimocidin, silver sulfadiazine, sulconazole, terbinafine, terconazole, tioconazole, tolnaftate, undecylenic acid, vacidin A, and voriconazole, while the compound of formula (XV), (XVI), or (XVII) (as described herein) is chosen from: disulfiram (bis(diethylthiocarbamoyl)disulfide), bis(dimethylthiocarbamoyl)disulfide, bis(dipropylthiocarbamoyl)disulfide, bis(dibutylthiocarbamoyl)disulfide, bis(dipentylthiocarbamoyl)disulfide, bis(di(2-methylpropyl)thiocarbamoyl) disulfide, bis(piperidinothiocarbamoyl)disulfide, bis(morpholinothiocarbamoyl)disulfide, bis((4-methylpiperazino)thiocarbamoyl)disulfide, bis((4-(2-hydroxyethyl)piperazino)thiocarbamoyl)disulfide, bis((hexahydro-4-methyl-1H-1,4-diazepin-1-yl)thiocarbamoyl)disulfide, and bis(3,3-dimethylcarbazoyl)disulfide.

The combination of an antifungal drug, such as amphotericin B, and a dithiocarbamoyl disulfide, such as disulfiram, has antifungal activity greater than that of either amphotericin B or disulfiram alone. Thus, combinations of disulfiram and amphotericin B may also be useful for the treatment of fungal infections. In addition, the using these two agents in combination has potential to mitigate side effects that could be encountered by using amphotericin B alone at high doses.

By “antifungal agent” is meant an agent that reduces or inhibits the growth of a fungus by at least 10%, relative to an untreated control, with the proviso that the agent does not belong to the dithiocarbamoyl disulfide class of compounds. Exemplary antifungal agents are provided herein.

Amphotericin B

Amphotericin B is a polyene antibiotic isolated from Streptomyces nodosus. It contains a macrolide ring and an aminosugar, mycosamine. The formula of amphotericin B is:

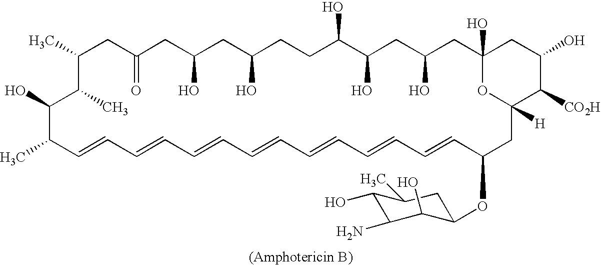

Amphotericin B is currently used for a wide range of systemic fungal infections and is formulated for IV injection and administered in this manner or intrathecally. Amphotericin B is poorly water soluble, but is sufficiently soluble that it is administered by IV infusion (0.1 mg/mL) or (0.3 mg/mL) in 5% dextrose. Amphotericin B is unstable in solution, particularly in normal saline. Other polyene macrolide antifungal agents include nystatin, candidin, rimocidin, vacidin A, and pimaricin.

Other Antifungal Agents

Antifungal agents are known that derive their mechanism of action by their inhibition of cytochrome-P450 activity, which decreases conversion of 14-alpha-methylsterols to ergosterol. Failure of ergosterol synthesis causes altered membrane permeability leading to loss of ability to maintain a normal intracellular environment. Examples of antifungal agents that inhibit ergosterol biosynthesis by their cytochrome-P450 activity are fluconazole, itraconazole, ketoconazole, clotrimazole, butoconazole, econazole, ravuconazole, oxiconazole, posaconazole, sulconazole, terconazole, tioconazole, and voriconazole. Other antifungal agents that are egosterol biosynthesis inhibitors act by blocking squalene epoxidation. Examples of antifungal agents that inhibit ergosterol biosynthesis by blocking squalene epoxidation are amorolfine, butenafine, naftifine, and terbinafine.

Flucytosine is an antifungal agent that is known to derive its mechanism of action by its antimetabolic activity. It is converted to 5-fluorouracil (5-FU), which inhibits thymidylate synthetase and thereby inhibits fungal protein synthesis.

Griseofulvin is an antifungal agent that inhibits fungal mitosis by disrupting the mitotic spindle through its interaction with polymerized microtubules.

Antifungal agents are also known that serve as glucan synthesis inhibitors. Glucan is a key component of the fungal cell wall, and inhibition of this enzyme produces significant antifungal effects. Examples of glucan synthesis inhibitors are caspofungin, micafungin, and anidulafungin.

Disulfiram, or another dithiocarbamoyl disulfide, may be used in combination with any of the foregoing antifungal agents such that the dose of the antifungal agent is lowered and any side effects resulting from its mechanism of action mitigated.

Dithiocarbamoyl Disulfides

Disulfiram [bis(diethylthiocarbamoyl)disulfide] is a member of the dithiocarbamoyl disulfide class of compounds. It occurs as a white to off-white, odorless, and almost tasteless powder, soluble in water to the extent of about 20 mg/100 mL, and in alcohol to the extent of about 3.8 mg/100 mL. It is currently formulated for oral administration, with each tablet containing 250 mg or 500 mg of disulfiram. Its formula is:

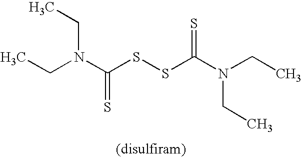

Some analogs of disulfiram have the following formulae:

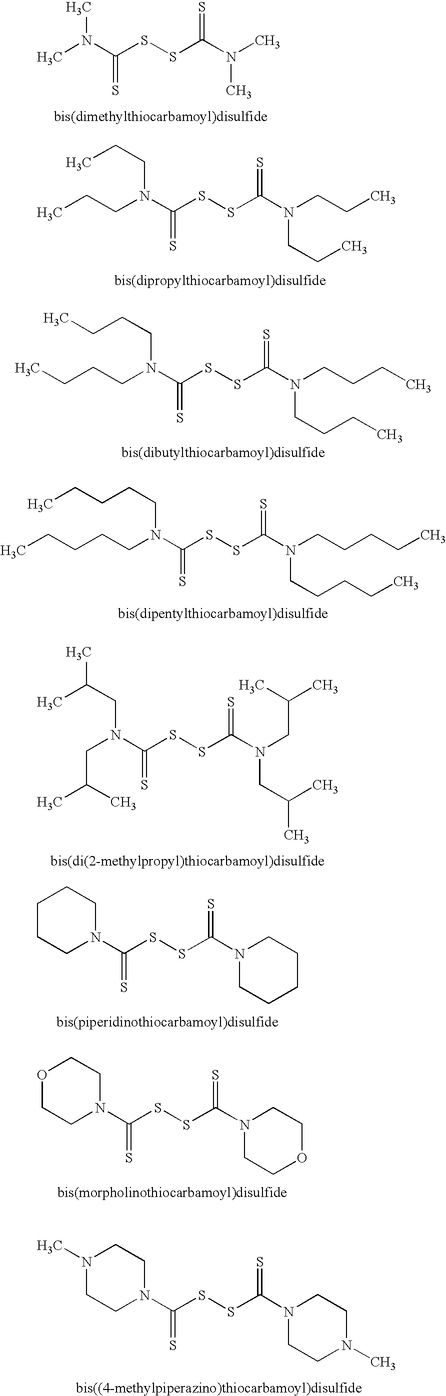

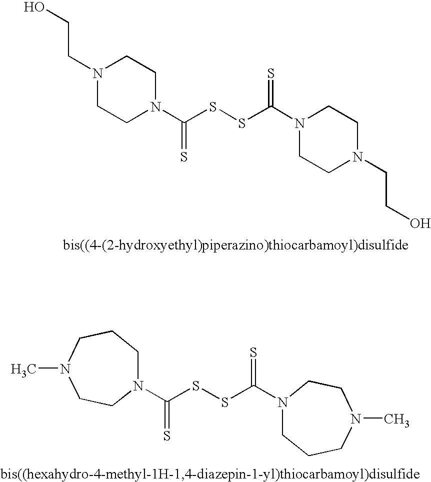

Dithiocarbamoyl disulfide compounds also include analogs that have structures of the following formulas (XV), (XVI), and (XVII): 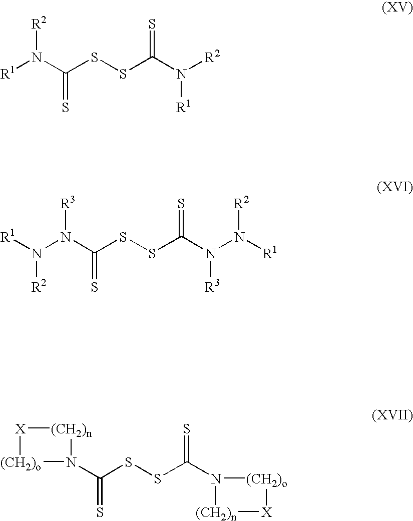
wherein X is CH₂, O, S, NR⁴, N(CH₂)ₚOR⁵, CH(CH₂)_{q}OR⁶, CH(CH₂)ᵣCO₂R⁷, CH(CH₂)ₛCONR⁸R⁹, 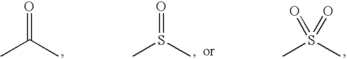
where R¹ and R² are independently C₁-C₈ linear or branched alkyl, alkaryl, or aryl, R³, R⁴, R⁵, R⁶, R⁷, R⁸, and R⁹ are independently H, C₁-C₈ linear or branched alkyl, alkaryl, or aryl, n is 0-3, o is 2-4, p is 2-6, and q, r, or s is 0-6.

By “aromatic residue” is meant an aromatic group having a ring system with conjugated π electrons (e.g., phenyl, or imidazole ). The ring of the aryl group preferably has 5 to 10 atoms. The aromatic ring may be exclusively composed of carbon atoms or may be composed of a mixture of carbon atoms and heteroatoms (i.e., nitrogen, oxygen, sulfur, and phosphorous). Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halo, fluoroalkyl, carboxyl, carboxyalkyl, amino, aminoalkyl, monosubstituted amino, disubstituted amino, and quaternary amino groups.

The term “aryl” means mono or bicyclic aromatic or heteroaromatic rings or ring systems. Examples of aryl groups include phenyl, naphthyl, pyrrolyl, furanyl, indolyl, benzofuranyl, benzothiophenyl, imidazolyl, triazolyl, tetrazolyl, benzimidazolyl, oxazolyl, benzoxazolyl, thiazolyl, benzothiazolyl, pyrazolyl, benzopyrazolyl, isoxazolyl, benzisoxazolyl, isothiazolyl, benzisothiazolyl, pyridinyl, quinolinyl, and isoquinolinyl.

“Heterocyclyl” means non-aromatic rings or ring systems that contain at least one ring hetero atom (e.g., O, S, N, P). Heterocyclic groups include, for example, pyrrolidinyl, tetrahydrofuranyl, morpholinyl, thiazolidinyl, and imidazolidinyl groups.

Aryl and heterocyclyl groups may be unsubstituted or substituted by one or more substituents selected from the group consisting of C₁₋₁₀ alkyl, hydroxy, halo, nitro, C₁₋₁₀ alkoxy, C₁₋₁₀ alkylthio, trihalomethyl, C₁₋₁₀ acyl, arylcarbonyl, heteroarylcarbonyl, nitrile, C₁₋₁₀ alkoxycarbonyl, oxo, arylalkyl (wherein the alkyl group has from 1 to 10 carbon atoms) and heteroarylalkyl (wherein the alkyl group has from 1 to 10 carbon atoms).

Compounds useful in the drug combinations described herein include those described herein in any of their pharmaceutically acceptable forms, including racemic mixtures and substantially pure isomers (e.g., diastereomers, enantiomers) of compounds described herein, as well as salts, solvates, and polymorphs thereof.

Pharmaceutically acceptable salts of disulfiram and related dithiocarbamoyl disulfides are also useful compounds of the invention, as are metal chelates of these compounds. Preferred metals include, for example, copper, manganese, iron, and zinc.

Drug Combination Comprising an Antifungal Compound and a Manganese Compound

In certain embodiments, the drug combination that has anti-scarring activity comprises at least two agents, wherein at least one agent is an antifungal drug, such as an allylamine, and at least one second agent is a manganese compound. In a specific embodiment, the allylamine compound is terbinafine. In certain embodiments, the manganese compound is manganese sulfate or manganese chloride. In a specific embodiment, the drug combination comprises terbinafine and manganese sulfate. In certain embodiments, the anti-fungal agent is selected from terbinafine, N-(5, 5-dimethylhex-3-yn-1-yl)-N-methyl-1-naphthalenemethanamine, (E)-N-(6,6-dimethyl-2-hepten-4-ynyl)-N-(iminomethyl)-1-naphthalenemethanamine, (E)-N-(6,6-dimethyl-2-hepten-4-ynyl)-N-(1-iminoethyl)-1-naphthalenemethanamine, (Z)-N-(3-chloro-6,6-dimethyl-2-hepten-4-ynyl)-N-methyl-1-naphthalenemethanamine, and N-methyl-N-propargyl-2-aminotetralin. In another embodiment, the antifungal agent is selected from fluconazole, itraconazole, ketoconazole, posaconazole, ravuconazole, voriconazole, clotrimazole, econazole, miconazole, oxiconazole, sulconazole, terconazole, and tioconazole. In a certain particular embodiment, the antifungal agent is haloprogin. In certain embodiments, the drug combination further comprises an antibacterial agent selected from tetracyclines, macrolides, lincosamides, ketolides, fluoroquinolones, glycopeptide antibiotics, and polymyxin antibiotics or analog thereof. In a certain embodiment, the antibacterial agent is selected from gentamicin, amikacin, kanamycin, framycetin, neomycin, netilmicin, streptomycin, and tobramycin. In another embodiment, the antibacterial agent is selected from silver sulfadiazine, sodium sulfacetamide, sulfamethoxazole, sulfanilamide sulfasalazine, sulfisoxazole, trimethoprim, sulfamethoxazole, and triple sulfa.

Terbinafine is a synthetic antifungal agent that inhibits ergosterol biosynthesis via inhibition of squalene epoxidase, an enzyme part of the fungal sterol synthesis pathway that creates the sterols needed for the fungal cell membrane. In vitro, terbinafine has activity against most Candida spp., Aspergillus spp., Sporothrix schenckii, Penicillium marneffei, Malassezia furfur, Cryptococcus neoformans, Trichosporon spp. and Blastoschizomyces.

In addition to terbinafine, allylamines include amorolfine, butenafine, naftifine, N-(5, 5-dimethylhex-3-yn-1-yl)-N-methyl-1-naphthalenemethanamine, (E)-N-(6,6-dimethyl-2-hepten-4-ynyl)-N-(iminomethyl)-1-naphthalenemethanamine, (E)-N-(6,6-dimethyl-2-hepten-4-ynyl)-N-(1-iminoethyl)-1-naphthalenemethanamine, (Z)-N-(3-chloro-6,6-dimethyl-2-hepten-4-ynyl)-N-methyl-1- naphthalenemethanamine, and N-methyl-N-propargyl-2-aminotetralin, some of which are shown in the table 3 below.

TABLE 3

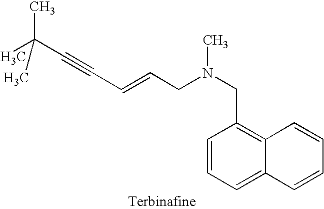

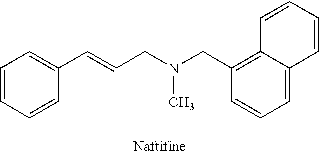

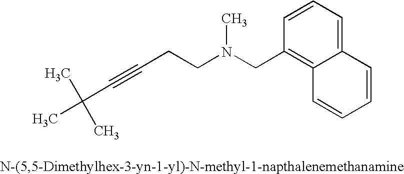

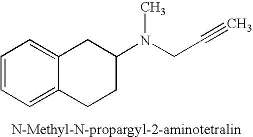

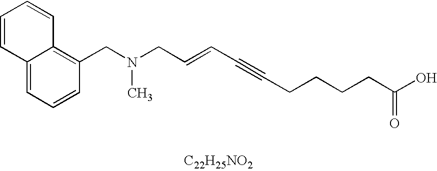

Other allylamine or allylamine analogs that can be used in the methods, kits, and compositions of the invention are described in U.S. Pat. Nos. 4,202,894; 4,282,251; 4,751,245; 4,755,534; 5,021,458; 5,132,459; 5,234,946; 5,334,628; 5,935,998; and 6,075,056.

Other Antifungal Agents

Other antifungal agents suitable for use in the drug combinations and related methods are described below. The antifungal azoles are preferred. Antifungal azoles are generally within in two classes, the imidizoles, such as miconazole, ketoconazole, and clotrimazole; and the triazoles, such as fluconazole, voriconazole, and ravuconazole. Other azoles are azaconazole, bromuconazole bitertanol, propiconazole, difenoconazole, diniconazole, cyproconazole, epoxiconazole, fluquinconazole, flusilazole, flutriafol, hexaconazole, itraconazole, imazalil, imibenconazole, ipconazole, tebuconazole, tetraconazole, fenbuconazole, metconazole, myclobutanil, perfurazoate, penconazole, posaconazole, pyrifenox, prochloraz, terconazole, triadimefon, triadimenol, triflumizole, and triticonazole.

Exemplary antifungal agents are selected from fluconazole, itraconazole, ketoconazole, posaconazole, ravuconazole, voriconazole, clotrimazole, econazole miconazole, oxiconazole, sulconazole, terconazole, tioconazole, nikkomycin Z, caspofungin, micafungin (FK463), anidulafungin (LY303366), amphotericin B (AmpB), AmpB lipid complex, AmpB colloidal dispersion, liposomal AmpB, liposomal nystatin, nystatin, pimaricin, lucensomycin, griseofulvin, ciclopirox olamine, haloprogin, tolnaftate, undecylenate, gentamicin, amikacin, kanamycin, framycetin, neomycin, netilmicin, streptomycin, tobramycin, silver sulfadiazine, sodium sulfacetamide, sulfamethoxazole, sulfanilamide sulfasalazine, sulfisoxazole, trimethoprim, sulfamethoxazole, triple sulfa, amrolfine, fenpropimorph, butenafine, and flucytosine.

Manganese Compounds

As used herein, a “manganese compound” is any salt or a complex of manganese. By “manganese salt” is meant any compound that results from replacement of part or all of the acid hydrogen of an acid by manganese. Manganese salts include, without limitation, acetate, adipate, alginate, ascorbate, aspartate, benzoate, bicarbonate, borate, butyrate, camphorate, carbonate, chlorate, clorite, citrate, cyanate, digluconate, fumarate, glucoheptanoate, glutamate, glycerophosphate, heptanoate, hexanoate, hydroxide, hypochlorite, lactate, maleate, nicotinate, nitrate, nitrite, oxalate, oxide, palmitate, pamoate, pectinate, perchlorate, peroxide, 3-phenylpropionate, phosphate, hydrogen phosphate, dihydrogen phosphate, phosphite, picrate, pivalate, propionate, salicylate, suberate, succinate, tartrate, triiodide, bromide, chloride, fluoride, and iodide. The salt can be the manganese salt of a metal complex, e.g., manganese(II) zinc bis(dithiocarbamate) (also known as Mancozeb). Preferred manganese salts are those of sulfur-containing anions including, without limitation, sulfide, sulphite, sulfate, bisulfate, bisulfite, persulfate, thiosulfate, hyposulfite, undecanoate sulfate, thiocyanate, benzenesulfonate, 2-hydroxyethanesulfonate, dodecylsulfate, hemisulfate, methanesulfonate, 2-naphthalenesulfonate, tosylate, ethanesulfonate, and camphorsulfonate. Desirably, the manganese compound is manganese sulfate or manganese chloride. Specifically excluded from the definition of “manganese compound” is manganese when present in food.

By “manganese complex” is meant a manganese compound including one or more chelate rings wherein the ring includes a manganese atom. Desirably, the complex is a macrocyclic or polydentate complexes of manganese. Manganese complexes include, without limitation, complexes of phenanthroline, 8-quinolinol, 2,6-diaminopyridine, bipyridine, diethylenetriamine, DPDP, EDDA, EDTA, EDTP, EDTA-BMA, DTPA, DOTA, DO3A, acetylacetonate, azamacrocycles, porphyrins, and Schiff-base complexes.

Manganese complexes include those complexes described in U.S. Pat. Nos. 6,541,490, 6,525,041, 6,204,259, 6,177,419, 6,147,094, 6,084,093, 5,874,421, 5,637,578, 5,610,293, 5,246,847, 5,155,224, 4,994,259, 4,978,763, 4,935,518, 4,654,334, and 4,478,935. Binuclear, trinuclear, and tetranuclear complexes of manganese can also be used. Preferably, the manganese complex is a complex of ethylene-bis-dithiocarbamate. Most preferably, the manganese complex is manganese(II) ethylene bis(dithiocarbamate) (also known as Maneb). Methods for preparing manganese complexes are described in, for example, U.S. Pat. No. 5,155,224 and by F. A. Cotton and G. Wilkinson “Advanced Inorganic Chemistry,” John Wiley & Sons, 5^{th} Ed. (1988).

The manganese compounds described herein can be selected from any oxidation state (e.g., Mn(0) to Mn(VII)). In certain specific embodiments, the manganese compound is a manganous (e.g., Mn(II) compounds) or manganic (e.g., Mn(III)) salt or complex.

Additional Agents

When the manganese compound is incorporated as an enhancer in the formulation of an antifungal compound, it is desirable to include additional agents. The term “enhancer” as used herein refers to heightened or increased, especially, increased or improved quality or desirability of the combination of compounds. Thus, in some of the instances, the manganese compound may act as an enhancer of antifungal activity of a combination of antifungal agents. For example, when the manganese compound is used in combination with an allylamine-derived antifungal agent, such as terbinafine, or an azole-derived antifungal agent, such as fluconazole, itraconazole, or caspofungin, the manganese compound enhances the antifungal activity of these compounds against C. glabrata, thereby acting as an enhancer.

The additional agent administered may be any compound that is suitable for intravenous, rectal, oral, topical, intravaginal, ophthalmic, or inhalation administration. Preferably, such agents are administered to alleviate other symptoms of the disease or for co-morbid conditions. In general, this includes: antibacterial agents (e.g., sulfonamides, antibiotics, tetracyclines, aminoglycosides, macrolides, lincosamides, ketolides, fluoroquinolones, glycopeptide antibiotics, and polymyxin antibiotics); analgesic agents; antidiarrheals; antihelminthics; anti-infective agents such as antibiotics and antiviral agents; antifungal agents; antinauseants; antipruritics; antitubercular agents; antiulcer agents; antiviral agents; cough and cold preparations, including decongestants; diuretics; genetic materials; herbal remedies; nutritional agents, such as vitamins, essential amino acids and fatty acids; ophthalmic drugs such as antiglaucoma agents. Administration of the antifungal agent and manganese compound can be administered before, during, or after administration of one or more of the above agents.

For example, administration of a drug combination as described herein can be administered before, during, or after administration of one or more antibacterial agents. Exemplary antibacterial agents that can be administered include β-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); β-lactamase inhibitors (e.g., clavulanate, sulbactam, and tazobactam); tetracyclines (e.g., tetracycline, chlortetracycline, demeclocycline, minocycline, oxytetracycline, methacycline, and doxycycline); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); metronidazole; daptomycin; garenoxacin; ramoplanin; faropenem; polymyxin; tigecycline, AZD2563; and trimethoprim. These antibacterial agents can be used in the dose ranges currently known and used for these agents. Different concentrations may be employed depending, e.g., on the clinical condition of the patient, the goal of therapy (treatment or prophylaxis), the anticipated duration, and the severity of the infection for which the drug is being administered. Additional considerations in dose selection include the type of infection, age of the patient (e.g., pediatric, adult, or geriatric), general health, and comorbidity. Determining what concentrations to employ are within the skills of the pharmacist, medicinal chemist, or medical practitioner. Typical dosages and frequencies are provided, e.g., in the Merck Manual of Diagnosis & Therapy (17th Ed. MH Beers et al., Merck & Co.).

A drug combination described herein can also be administered along with an antiprotozoal agent, such as pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl) thiophene-bis-O-methylamidoxime, 2,4-bis(4-amidinophenyl)thiophene, or 2,4-bis(4-amidinophenyl)thiophene-bis-O-methylamidoxime.

Chelating agents can also be used with an antifungal agent and a manganese compound in the methods, compositions, and kits of the invention. Chelating agents include phosphonic acids, methylenglycine diacetic acid, iminodisuccinate, glutamate, N, N-bis(carboxymethyl, S,S′-ethylenediamine disuccinic acid (EDDS), β-alaninediacetic acid, ethylenediamine-N,N,N′,N′,-tetraacetic acid, ethylenediamine-N,N,N′,N′,-tetraacetic acid, disodium salt, dihydrate, ethylenediamine-N,N,N′,N′,-tetraacetic acid, trisodium salt, trihydrate, ethylenediamine-N,N,N′,N′-tetraacetic acid, tetrasodium salt, tetrahydrate, ethylenediamine-N,N,N′,N′-tetraacetic acid, dipotassium salt, dihydrate, ethylenediamine-N,N,N′,N′-tetraacetic acid, dilithium salt, monhydrate, ethylenediamine-N,N,N′,N′-tetraacetic acid, diammonium salt, ethylenediamine-N,N,N′,N′-tetraacetic acid, tripotassium salt, dihydrate, ethylenediamine-N,N,N′,N′-tetraacetic acid, ethylenediamine-N,N,N′,N′-tetraacetic acid, calcium chelate, ethylenediamine-N,N,N′,N′-tetraacetic acid, cerium chelate, ethylenediamine-N,N,N′,N′-tetraacetic acid, ethylenediamine-N,N,N′,N′-tetraacetic acid, ethylenediamine-N,N,N′,N′-tetraacetic acid, dysprosium chelate, ethylenediamine-N,N,N′,N′-tetraacetic acid, europium chelate, ethylenediamine-N,N,N′,N′-tetraacetic acid, iron chelate, ethylenediamine-N,N,N′,N′-tetraacetic acid, ethylenediamine-N,N,N′,N′-tetraacetic acid, ethylenediamine-N,N,N′,N′-tetraacetic acid, ethylenediamine-N,N,N′,N′-tetraacetic acid, ethylenediamine-N,N,N′,N′-tetraacetic acid, ethylenediamine-N,N,N′,N′-tetraacetic acid, samarium chelate, ethylenediamine-N,N,N′,N′-tetraacetic acid, ethylenediamine-N,N,N′,N′-tetraacetic acid, zinc chelate, trans-1,2-diaminocyclohexane-N,N,N′,N′-tetraaceticacid, monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid dihydrochloride, ethylenediamine-N,N′-bis(methylenephosphonic acid), hemihydrate, N-(2-hydroxyethyl)ethylenediamine-N,N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, barium chelate, cobalt chelate, copper chelate, indium chelate, lanthanum chelate, magnesium chelate, nickel chelate, strontium chelate, nitrilotripropionic acid, dimercaprol (2,3-dimercapto-1-propanol), nitrilotris(methylenephosphoric acid), trisodium salt, 7,19,30-trioxa-1,4,10,13,16,22,27,33-octaazabicyclo[11,11,11]pentatriacontane hexahydrobromide, and triethylenetetramine-N,N,N′,N″,N′″,N′″-hexaacetic acid. When the chelating agent is used in combination with an antifungal agent and a manganese compound, there is desirably a decrease in the consumption of either the antifungal agent or the manganese compound, or both.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

Combinations Comprising Ciclopirox and Antiproliferative Agents

In certain embodiments, the drug combinations according to the present invention may comprise ciclopirox (or its structural or functional analogs, salts or metabolites) and an antiproliferative agent.

Ciclopirox

Ciclopirox (6-cyclohexyl-1-hydroxy-4-methyl-2(1H)-pyridinone) is a synthetic antifungal agent having a broad spectrum of activity. It can be fungistatic and fungicidal against species including, for example, Candida albicans, Trichophyton spp., Epidermophyton spp., and Aspergillus spp. Antibacterial properties of ciclopirox have also been demonstrated against both Gram-positive and Gram-negative species (Abrams et al., Clin. Dermatol., 9: 471-477, 1992). Anti-inflammatory activity including the inhibition of prostaglandin and leukotriene synthesis in human polymorphonuclear cells has also been reported.

Ciclopirox Analogs

Structural and functional analogs (e.g., mimosine) can replace ciclopirox in the therapeutic combinations of this invention. Structural ciclopirox analogs may be 2-pyridinones of general structure: 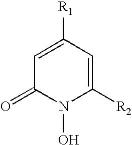
wherein R₁ is H, OH, NH₂, a halide, or any branched or unbranched, substituted or unsubstituted C₁₋₁₀ alkyl, C₁₋₁₀ alkoxyalkyl, C₁₋₁₀ hydroxyalkyl, C₁₋₁₀ aminoalkyl, C₁₋₁₀ alkylaminoalkyl, C₄₋₁₀ cycloalkyl, C₅₋₈ aryl, or C₆₋₂₀ alkylaryl, and R₂ is H, OH, NH₂, a halide, or any branched or unbranched, substituted or unsubstituted C₁₋₁₀ alkyl, C₁₋₁₀ alkoxyalkyl, C₁₋₁₀ hydroxyalkyl, C₁₋₁₀ aminoalkyl, C₁₋₁₀ alkylaminoalkyl, C₄₋₁₀ cycloalkyl, C₅₋₈ aryl, C₆₋₂₀ alkylaryl, C₃₋₁₀ heterocyclyl, or C₃₋₁₀ heteroaryl, wherein 1-4 carbon atoms of any of R₁ or R₂ may be substituted with one or more heteroatoms. Particularly useful R₁ groups include H, CH₃, CH₃CH₂, (CH₃)₂CH, (CH₃CH₂)₂CH, CH₃O, CH₃CH₂O, (CH₃)₂CHO, and (CH₃CH₂)₂CHO. Particularly useful R₂ groups include cyclopentyl, cyclohexyl, CH₂CH(CH₃)CH₂C(CH₃)₃, and 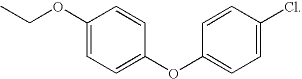
Particularly useful 2-pyridinones analogs, in addition to ciclopirox (R₁═CH₃; R₂═cyclohexyl), include octopirox (R₁═CH₃; R₂═CH₂CH(CH₃)CH₂C(CH₃)₃), and rilopirox (R₁═CH₃; R₂═

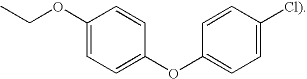

Methods for synthesizing 2-pyridinone derivatives are well known in the art (see, for example, U.S. Pat. Nos. 3,883,545 and 3,972,888).

Functional ciclopirox analogs, useful for combination therapy according to this invention, inhibit DNA initiation at origins of replication, are not purines or pyrimidines, and do not replace naturally occurring nucleotides during DNA synthesis. Functional ciclopirox analogs include, for example, mimosine and geminin. Inhibition of DNA initiation at origins of replication can be easily assessed using standard techniques. For example, replication intermediates isolated from cells cultured in the presence of the candidate ciclopirox analog can be assessed by 2D gel electrophoresis (Levenson et al., Nucleic Acid Res., 17: 3997-4004, 1993). This method takes advantage of the different electrophoretic properties of DNA fragments containing replication forks, replication bubbles, or termination structures. Fragments containing origins of replication are easily identified.

Antiproliferative Agents

“Antiproliferative agent” refers to a compound that, individually, inhibits the growth of a neoplasm. Antiproliferative agents include, but are not limited to microtubule inhibitors, topoisomerase inhibitors, platins, alkylating agents, and anti-metabolites.

By “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

Particular antiproliferative agents include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine. Additional antiproliferative agents are listed in Table 4 below.

In certain embodiments, antiproliferative agents are paclitaxel, gemcitabine, doxorubicin, vinblastine, etoposide, 5-fluorouracil, or carboplatin.

TABLE 4
A

| | | |
|---|---|---|
| Alkylating agents | cyclophosphamide | lomustine |
| | busulfan | procarbazine |
| | ifosfamide | altretamine |
| | melphalan | estramustine phosphate |
| | hexamethylmelamine | mechlorethamine |
| | thiotepa | streptozocin |
| | chlorambucil | temozolomide |
| | dacarbazine | semustine. |
| | carmustine | |
| Platinum agents | cisplatin | carboplatinum |
| | oxaliplatin | ZD-0473 (AnorMED) |
| | spiroplatinum, | lobaplatin (Aeterna) |
| | carboxyphthalatoplatinum, | satraplatin (Johnson Matthey) |
| | tetraplatin | BBR-3464 (Hoffmann-La Roche) |
| | ormiplatin | SM-11355 (Sumitomo) |
| | iproplatin | AP-5280 (Access) |
| Antimetabolites | azacytidine | tomudex |
| | gemcitabine | trimetrexate |
| | capecitabine | deoxycoformycin |
| | 5-fluorouracil | fludarabine |
| | floxuridine | pentostatin |
| | 2-chlorodeoxyadenosine | raltitrexed |
| | 6-mercaptopurine | hydroxyurea |
| | 6-thioguanine | decitabine (SuperGen) |
| | cytarabin | clofarabine (Bioenvision) |
| | 2-fluorodeoxy cytidine | irofulven (MGI Pharma) |
| | methotrexate | DMDC (Hoffmann-La Roche) |
| | idatrexate | ethynylcytidine (Taiho) |
| Topoisomerase | amsacrine | rubitecan (SuperGen) |
| inhibitors | epirubicin | exatecan mesylate (Daiichi) |
| | etoposide | quinamed (ChemGenex) |
| | teniposide or mitoxantrone | gimatecan (Sigma-Tau) |
| | irinotecan (CPT-11) | diflomotecan (Beaufour-Ipsen) |
| | 7-ethyl-10-hydroxy-camptothecin | TAS-103 (Taiho) |
| | topotecan | elsamitrucin (Spectrum) |
| | dexrazoxanet (TopoTarget) | J-107088 (Merck & Co) |
| | pixantrone (Novuspharma) | BNP-1350 (BioNumerik) |
| | rebeccamycin analogue (Exelixis) | CKD-602 (Chong Kun Dang) |
| | BBR-3576 (Novuspharma) | KW-2170 (Kyowa Hakko) |
| Antitumor antibiotics | dactinomycin (actinomycin D) | amonafide |
| | doxorubicin (adriamycin) | azonafide |
| | deoxyrubicin | anthrapyrazole |
| | valrubicin | oxantrazole |
| | daunorubicin (daunomycin) | losoxantrone |
| | epirubicin | bleomycin sulfate (blenoxane) |
| | therarubicin | bleomycinic acid |
| | idarubicin | bleomycin A |
| | rubidazone | bleomycin B |
| | plicamycinp | mitomycin C |
| | porfiromycin | MEN-10755 (Menarini) |
| | cyanomorpholinodoxorubicin | GPX-100 (Gem Pharmaceuticals) |
| | mitoxantrone (novantrone) | |
| Antimitotic | paclitaxel | SB 408075 (GlaxoSmithKline) |
| agents | docetaxel | E7010 (Abbott) |
| | colchicine | PG-TXL (Cell Therapeutics) |
| | vinblastine | IDN 5109 (Bayer) |
| | vincristine | A 105972 (Abbott) |
| | vinorelbine | A 204197 (Abbott) |
| | vindesine | LU 223651 (BASF) |
| | dolastatin 10 (NCI) | D 24851 (ASTAMedica) |
| | rhizoxin (Fujisawa) | ER-86526 (Eisai) |
| | mivobulin (Warner-Lambert) | combretastatin A4 (BMS) |
| | cemadotin (BASF) | isohomohalichondrin-B (PharmaMar) |
| | RPR 109881A (Aventis) | ZD 6126 (AstraZeneca) |
| | TXD 258 (Aventis) | PEG-paclitaxel (Enzon) |
| | epothilone B (Novartis) | AZ10992 (Asahi) |
| | T 900607 (Tularik) | IDN-5109 (Indena) |
| | T 138067 (Tularik) | AVLB (Prescient NeuroPharma) |
| | cryptophycin 52 (Eli Lilly) | azaepothilone B (BMS) |
| | vinflunine (Fabre) | BNP-7787 (BioNumerik) |
| | auristatin PE (Teikoku Hormone) | CA-4 prodrug (OXiGENE) |
| | BMS 247550 (BMS) | dolastatin-10 (NIH) |
| | BMS 184476 (BMS) | CA-4 (OXiGENE) |
| | BMS 188797 (BMS) | |
| | taxoprexin (Protarga) | |
| Aromatase inhibitors | aminoglutethimide | exemestane |
| | letrozole | atamestane (BioMedicines) |
| | anastrazole | YM-511 (Yamanouchi) |
| | formestane | |
| Thymidylate synthase | pemetrexed (Eli Lilly) | nolatrexed (Eximias) |
| inhibitors | ZD-9331 (BTG) | CoFactor ™ (BioKeys) |
| DNA antagonists | trabectedin (PharmaMar) | mafosfamide (Baxter International) |
| | glufosfamide (Baxter International) | apaziquone (Spectrum |
| | albumin + 32P (Isotope Solutions) | Pharmaceuticals) |
| | thymectacin (NewBiotics) | O6 benzyl guanine (Paligent) |
| | edotreotide (Novartis) | |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A receptor | atrasentan (Abbott) | YM-598 (Yamanouchi) |
| antagonist | ZD-4054 (AstraZeneca) | |
| Retinoic acid receptor | fenretinide (Johnson & Johnson) | alitretinoin (Ligand) |
| agonists | LGD-1550 (Ligand) | |
| Immuno-modulators | interferon | dexosome therapy (Anosys) |
| | oncophage (Antigenics) | pentrix (Australian Cancer |
| | GMK (Progenics) | Technology) |
| | adenocarcinoma vaccine (Biomira) | ISF-154 (Tragen) |
| | CTP-37 (AVI BioPharma) | cancer vaccine (Intercell) |
| | IRX-2 (Immuno-Rx) | norelin (Biostar) |
| | PEP-005 (Peplin Biotech) | BLP-25 (Biomira) |
| | synchrovax vaccines (CTL Immuno) | MGV (Progenics) |
| | melanoma vaccine (CTL Immuno) | β-alethine (Dovetail) |
| | p21 RAS vaccine (Gem Vax) | CLL therapy (Vasogen) |
| Hormonal and | estrogens | prednisone |
| antihormonal agents | conjugated estrogens | methylprednisolone |
| | ethinyl estradiol | prednisolone |
| | chlortrianisen | aminoglutethimide |
| | idenestrol | leuprolide |
| | hydroxyprogesterone caproate | goserelin |
| | medroxyprogesterone | leuporelin |
| | testosterone | bicalutamide |
| | testosterone propionate; | flutamide |
| | fluoxymesterone | octreotide |
| | methyltestosterone | nilutamide |
| | diethylstilbestrol | mitotane |
| | megestrol | P-04 (Novogen) |
| | tamoxifen | 2-methoxyestradiol (EntreMed) |
| | toremofine | arzoxifene (Eli Lilly) |
| | dexamethasone | |
| Photodynamic agents | talaporfin (Light Sciences) | Pd-bacteriopheophorbide (Yeda) |
| | Theralux (Theratechnologies) | lutetium texaphyrin (Pharmacyclics) |
| | motexafin gadolinium | hypericin |
| | (Pharmacyclics) | |
| Tyrosine Kinase | imatinib (Novartis) | kahalide F (PharmaMar) |
| Inhibitors | leflunomide (Sugen/Pharmacia) | CEP-701 (Cephalon) |
| | ZD1839 (AstraZeneca) | CEP-751 (Cephalon) |
| | erlotinib (Oncogene Science) | MLN518 (Millenium) |
| | canertinib (Pfizer) | PKC412 (Novartis) |
| | squalamine (Genaera) | phenoxodiol () |
| | SU5416 (Pharmacia) | trastuzumab (Genentech) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMG-1C11 (ImClone) |
| | EKB-569 (Wyeth) | |

B
Miscellaneous agents

| | |
|---|---|
| SR-27897 (CCK A inhibitor, Sanofi-Synthelabo) | BCX-1777 (PNP inhibitor, BioCryst) |
| tocladesine (cyclic AMP agonist, Ribapharm) | ranpirnase (ribonuclease stimulant, Alfacell) |
| alvocidib (CDK inhibitor, Aventis) | galarubicin (RNA synthesis inhibitor, Dong-A) |
| CV-247 (COX-2 inhibitor, Ivy Medical) | tirapazamine (reducing agent, SRI International) |
| P54 (COX-2 inhibitor, Phytopharm) | N-acetylcysteine (reducing agent, Zambon) |
| CapCell ™ (CYP450 stimulant, Bavarian Nordic) | R-flurbiprofen (NF-kappaB inhibitor, Encore) |
| GCS-100 (gal3 antagonist, GlycoGenesys) | 3CPA (NF-kappaB inhibitor, Active Biotech) |
| G17DT immunogen (gastrin inhibitor, Aphton) | seocalcitol (vitamin D receptor agonist, Leo) |
| efaproxiral (oxygenator, Allos Therapeutics) | 131-I-TM-601 (DNA antagonist, TransMolecular) |
| PI-88 (heparanase inhibitor, Progen) | eflornithine (ODC inhibitor, ILEX Oncology) |
| tesmilifene (histamine antagonist, YM BioSciences) | minodronic acid (osteoclast inhibitor, Yamanouchi) |
| histamine (histamine H2 receptor agonist, Maxim) | indisulam (p53 stimulant, Eisai) |
| tiazofurin (IMPDH inhibitor, Ribapharm) | aplidine (PPT inhibitor, PharmaMar) |
| cilengitide (integrin antagonist, Merck KGaA) | rituximab (CD20 antibody, Genentech) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug, Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promotor, Procyon) |
| bortezomib (proteasome inhibitor, Millennium) | doranidazole (apoptosis promotor, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promotor, MAXIA) |
| midostaurin (PKC inhibitor, Novartis) | apomine (apoptosis promotor, ILEX Oncology) |
| bryostatin-1 (PKC stimulant, GPC Biotech) | urocidin (apoptosis promotor, Bioniche) |
| CDA-II (apoptosis promotor, Everlife) | Ro-31-7453 (apoptosis promotor, La Roche) |
| SDX-101 (apoptosis promotor, Salmedix) | brostallicin (apoptosis promotor, Pharmacia) |
| ceflatonin (apoptosis promotor, ChemGenex) | |

Exemplary Drug Combinations

In certain other embodiments, the drug combinations comprise rilopirox and paclitaxel, rilopirox and gemcitabine, rilopirox and doxorubicin, rilopirox and vinblastine, rilopirox and etoposide, rilopirox and 5-flurouracil, or rilopirox and carboplatin.

In certain other embodiments, the drug combinations comprise octopirox and paclitaxel, octopirox and gemcitabine, octopirox and doxorubicin, octopirox and vinblastine, octopirox and etoposide, octopirox and 5-flurouracil, or octopirox and carboplatin.

In certain other embodiments, the drug combinations comprise mimosine and paclitaxel, mimosine and gemcitabine, mimosine and doxorubicin, mimosine and vinblastine, mimosine and etoposide, mimosine and 5-flurouracil, or mimosine and carboplatin.

In certain other embodiments, the drug combinations comprise germinin and paclitaxel, germinin and gemcitabine, germinin and doxorubicin, germinin and vinblastine, germinin and etoposide, germinin and 5-flurouracil, or germinin and carboplatin.

In certain embodiments, the drug combinations comprise ciclopirox and paclitaxel, ciclopirox and gemcitabine, ciclopirox and doxorubicin, ciclopirox and vinblastine, ciclopirox and etoposide, ciclopirox and 5-flurouracil, or ciclopirox and carboplatin.

Combinations Comprising Niclosamide and Antiproliferative Agents

In certain embodiments, the drug combinations according to the present invention may comprise an antihelminthic agent (e.g., niclosamide or its structural or functional analogs, salts, or metabolites) and an antiproliferative agent.

Antihelminthic Agents

“Antihelminthic agent” refers to a compound that, individually, inhibits the growth of a parasitic worm. Desirably, growth rate is reduced by at least 20%, 30%, 50%, or even 70%. Examples of helminthes include cestodes, trematodes, nematodes, Fasciola, Schistosoma, planaria, filaria, and Trichinella.

Antihelminthic agents encompass a broad spectrum of modes of action which include: glutamate-gated chloride channel potentiating compounds such as ivermectin, abamectin, doramectin, moxidectin, niclofolan, and mylbemycin D; calcium permeability potentiators such as praziquantel; malate metabolism inhibitors such as diamphenethide; phosphoglycerate kinase and mutase inhibitors such as chlorsulon; and benzaniles (e.g., salicylanilide compounds).

Benzanilides

Benzanilides that can be used according to the methods of the invention include those that fit formula XVIII: 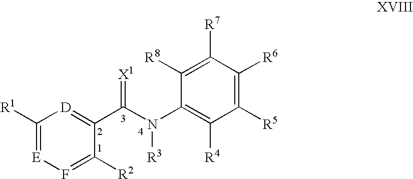
or a salt thereof. In formula XVIII, D is N or CR⁹; E is N or CR¹⁰; F is N or CR¹¹; and R¹ is H, halide, OR¹², SR¹³, NR¹⁴R¹⁵, or described by one of the formulas:

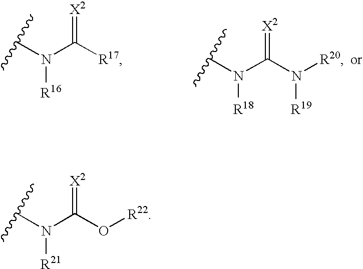

R² is H, OH, or OR¹²; R³ is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; or R² and R³ combine to form a six-membered ring in which position 1 is connected to position 4 by one of the groups:

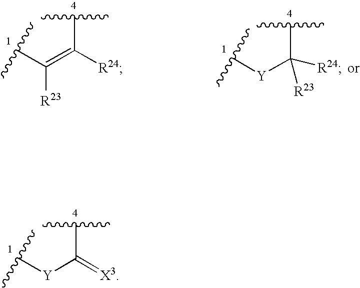

R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl; and R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, or are described by the formulas:

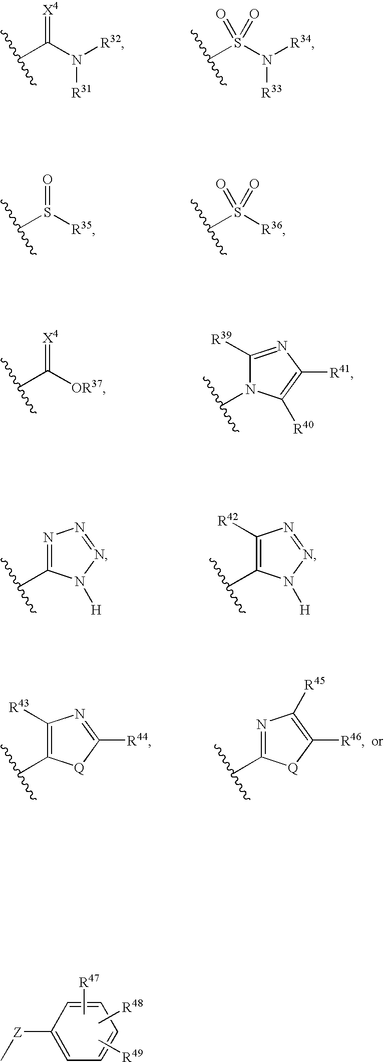

For compounds of formula XVIII, each X¹, X², X³, and X⁴ is, independently, O S; or NR³⁸; Y is CR²⁵R²⁶, O S, or NR²⁷; Z is O, S, or CR⁵⁰R⁵¹; each Q is, independently, O, S, or NR⁵²; R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂; R₁₂ and R¹³ are each, independently, acyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴, and R⁴⁷ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

In certain embodiments, X¹ is an oxygen atom; R² is OH; and R³ is H.

In certain other embodiments, X¹ is an oxygen atom; R² and R³ combine to form a six-membered ring in which position 1 is connected to position 4 by 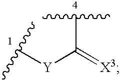
Y is an oxygen atom.

In certain other embodiments, X¹ is an oxygen atom; R² and R³ combine to form a six-membered ring in which position 1 is connected to position 4 by 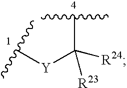
Y is an oxygen atom.

In certain embodiments, X¹ is an oxygen atom; R² is OH; D is CR⁹; E is CR¹⁰; F is CR¹¹; R¹ is halide; R¹¹ is hydrogen or halide; and R³, R⁹, and R¹⁰ are H.

Desirable compounds of formula XVIII are further described by any one of formulas XIX-XXII: 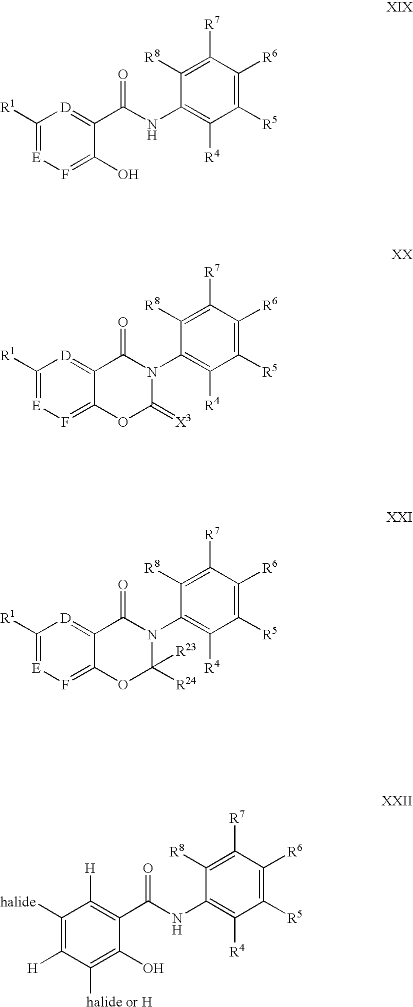
wherein F, E, D, X³, R¹, R⁴, R⁵, R⁶, R⁷, R⁸, R⁹, R¹⁰, R¹¹, R²³, and R²⁴ are as defined above.

Benzanilides that can be used according to the methods of the invention include various salicylanilides described in more detail below (e.g., niclosamide, oxyclozanide, closantel, resorantel, tribromsalan, clioxanide, dibromsalan, rafoxanide, flusalan), and the compounds disclosed in U.S. Pat. Nos. 3,041,236, 3,079,297, 3,113,067, 3,147,300, 3,332,996, 3,349,090, 3,449,420, 3,466,370, 3,469,006, 3,499,420, 3,798,258, 3,823,236, 3,839,443, 3,888,980, 3,906,023, 3,927,071, 3,949,075, 3,973,038, 4,005,218, 4,008,274, 4,072,753, 4,115,582, 4,159,342, 4,310,682, and 4,470,979, each of which is hereby incorporated by reference, Hlasta et al., Bioorg. Med. Chem., and European Patent No. 0533268. Salts or esters of any of these compounds can also be used according to the methods of the invention.

Salicylanilides

Salicylanilides consist of a salicylic acid ring and an anilide ring and are a subset of benzanilides. Exemplary salicylanilide compounds that can be used according to the present invention are depicted in the following Table 5.

TABLE 5

| | |
|---|---|
| 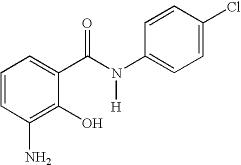 | 4′-chloro-3- nitrosalicylanilide |
| 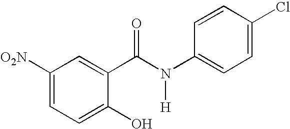 | 4′-chloro-5- nitrosalicylanilide |
| 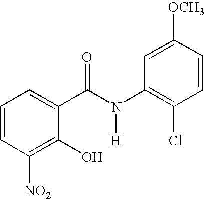 | 2′-chloro-5′-methoxy- 3-nitrosalicylanilide |
| 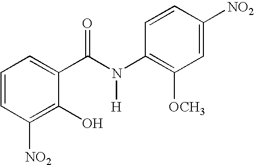 | 2′-methoxy-3,4′- dinitrosalicylanilide |
| 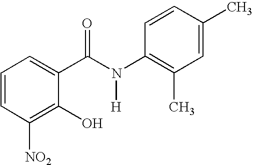 | 2′,4′-dimethyl- 3-nitrosalicylanilide |
| 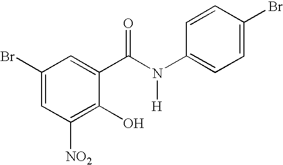 | 4′,5-dibromo-3- nitrosalicylanilide |
| 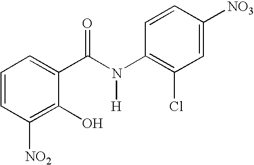 | 2′-chloro-3,4′- dinitrosalicylanilide |
| 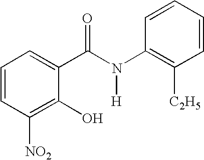 | 2′-ethyl-3- nitrosalicylanilide |
| 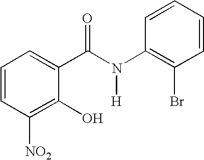 | 2′-bromo-3- nitrosalicylanilide |

Niclosamide

Niclosamide (2′,5-dichloro-4′-nitrosalicylanilide) is an antihelminthic used for treatment of cestode and trematode infestations in humans, pets, and, livestock. This drug has also been used as an effective lampricide and a pesticide against fresh water snails. The free base, the monohydrate, the ethanolamine salt, and the piperazine salt are know to be active as antihelmenthic agents. Niclosamide and its salts (e.g., the ethanolamine, piperazine, and monohydrate salts) exhibit very low toxicity in mammals. The structure of niclosamide and other benzanilide antihelmenthic agents are provided below. 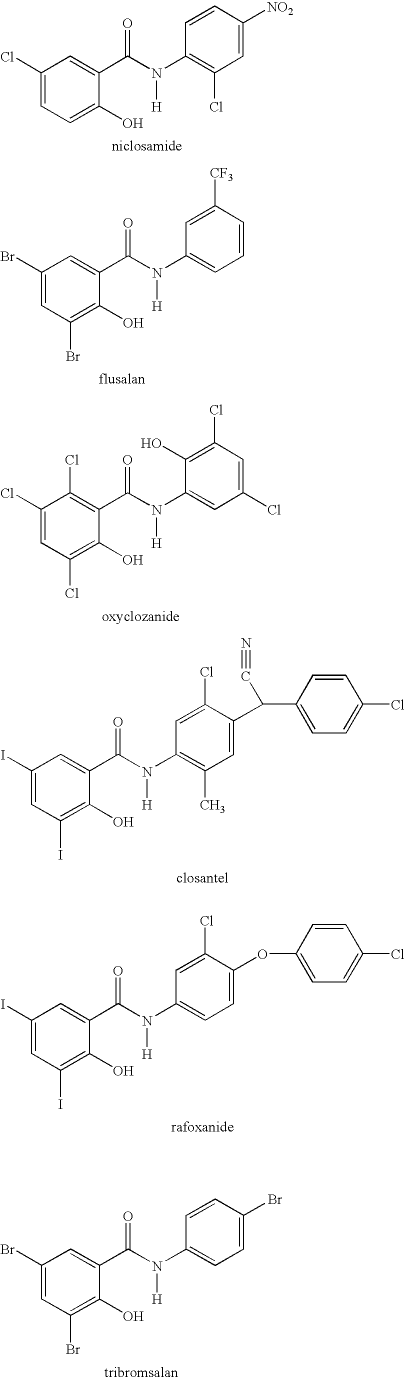 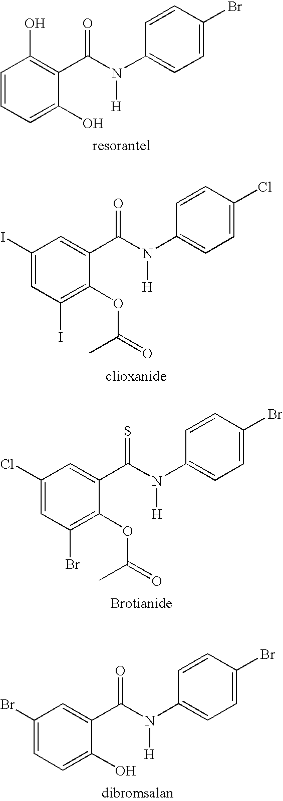
Synthetic Methods

Methods for synthesizing benzanilide and salicylanilide derivatives are well known in the art. For example, niclosamide and related compounds can be prepared as described in U.S. Pat. Nos. 3,079,297 and 3,113,067; flusalan and related compounds can be prepared as described in U.S. Pat. No. 3,041,236; oxyclozanide and related compounds can be prepared as described in U.S. Pat. No. 3,349,090; closantel and related compounds can be prepared as described in U.S. Pat. No. 4,005,218; resorantel and related compounds can be prepared as described in U.S. Pat. No. 3,449,420; tribromsalan, dibromsalan, and related compounds can be prepared as described in U.S. Pat. Nos. 2,967,885 and 3,064,048; clioxanide and related compounds can be prepared as described by Campbell et al., Experientia 23:992 (1967); and rafoxanide and related compounds can be prepared as described by Mrozak et al., Experientia 25:883 (1969). Additional methods are disclosed by, for example, Hlasta et al., Bioorg. Med. Chem., U.S. Pat. Nos. 3,466,370, 3,888,980, 3,973,038, 4,008,274, 4,072,753, and 4,115,582, and European Patent No. 0533268. All publications and patents mentioned above are incorporated herein by reference.

Compounds of formula XXI can be prepared, for example, by condensation of a salicylanilide with an aldehyde, see reaction 1, as described in Acta Pharmaceutica (Zagreb) 50:239 (2000); or by reaction with acetylene, see reaction 2, as described in Khimiya Geterotsiklicheskikh Soedinenii 4:469 (1983) or Khimiya Geterotsiklicheskikh Soedinenii 9:1278 (1979).

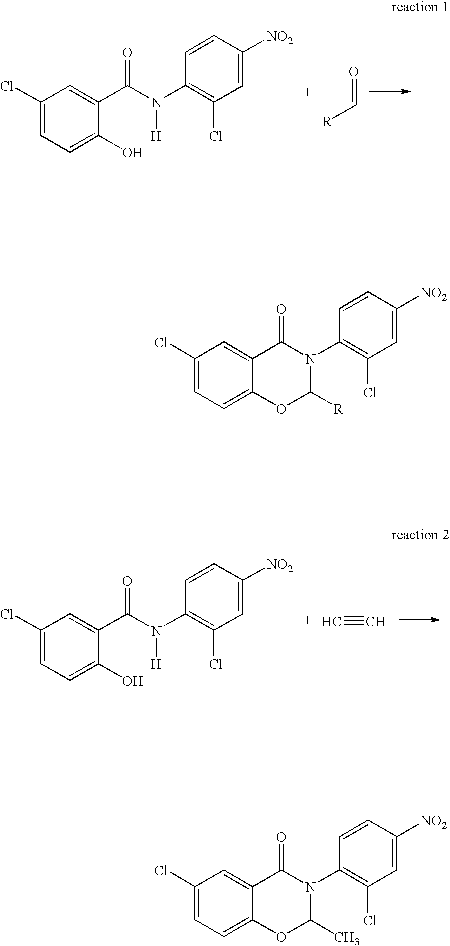

Compounds of formula XX in which X³ is an oxygen atom can be prepared, for example, by condensation of a salicylanilide with ethyl chloroformate, see reaction 3, as described in Pharmazie 45:34 (1990); J. Med. Chem. 32:807 (1989); or J. Med. Chem. 21:1178 (1978).

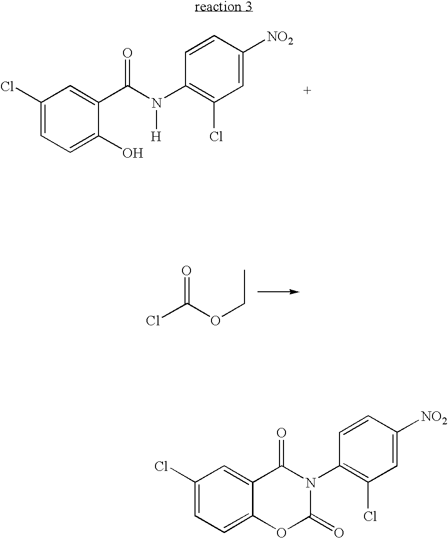

Compounds of formula XX in which X³ is a sulfur atom can be prepared, for example, by condensation of a salicylanilide with thiophosgene, see reaction 4, as described in Archiv der Pharmazie (Weinheim, Germany) 315:97 (1982); Indian J. Chem., Sect. B 18:352 (1979); Indian J. Chem., Sect. B15:73 (1977); or Indian J. Pharm., 37:133 (1975).

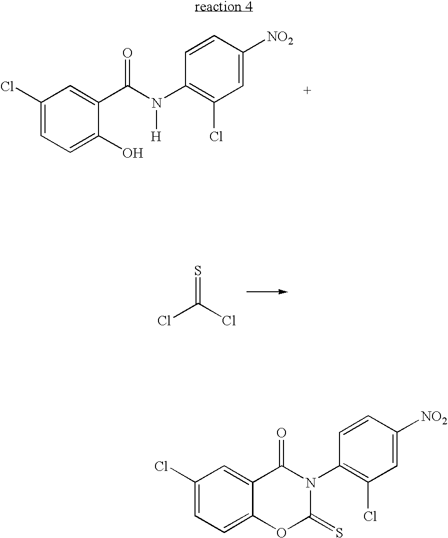

Compounds of formula XX in which X³ is NH can be prepared, for example, by reaction of a salicylanilide with cyanogen bromide, see reaction 5, as described in C. R. Hebd. Seances Acad. Sci., Ser. C 283:291 (1976).

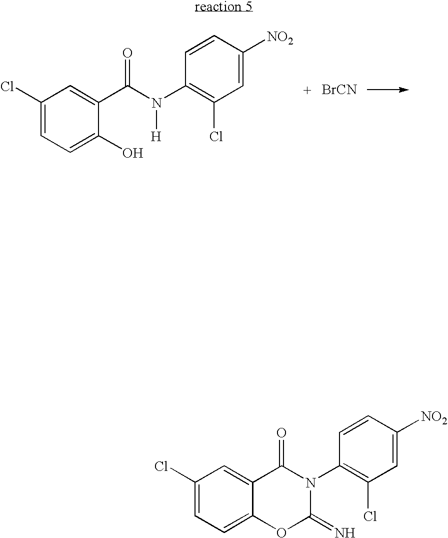

Compounds of formula XVIII in which D, E, or F is a nitrogen atom can be prepared using methods analogous to those used for the synthesis of salicylanilide compounds. For example, 2-hydroxynicotinic acid (Aldrich Cat. No. 25,105-4), 3-hydroxypicolinic acid (Aldrich Cat. No. 15,230-7), 6-hydroxynicotinic acid (Aldrich Cat. No. 12,875-9), 6-hydroxypicolinic acid (Aldrich Cat. No. 38,430-5), 5-chloro-6-hydroxynicotinic acid (Fluka Cat. No. 24882), 5-bromonicotinic acid (Aldrich Cat. No. 22843-5), 2-chloronicotinic acid (Aldrich Cat. No. 15,033-9), 6-chloronicotinic acid (Aldrich Cat. No. 15,635-3), 5,6-dichloronicotinic acid (Aldrich Cat. No. 34,021-9), or citrazinic acid (Aldrich Cat. No. 15,328-1) can be reacted with an aniline to produce a compound of formula XVIII in which D, E, or F are a nitrogen atom. Furthermore, 2-hydroxynicotinic acid derivatives and 3-hydroxypyrazine-2-carboxylic acid derivatives can be prepared using the methods described in U.S. Pat. Nos. 5,364,940, 5,516,661, and 5,364,939. For example, 5-chloronicotinic acid (CAS 22620-27-5) can be hydroxylated using the methods described in U.S. Pat. No. 5,364,940 and the resulting 2-hydroxy-5-chloronicotinic acid coupled with 2-chloro-4-nitroaniline (Aldrich Cat. No. 45,685-3), as shown in reaction 6, using standard amide coupling techniques.

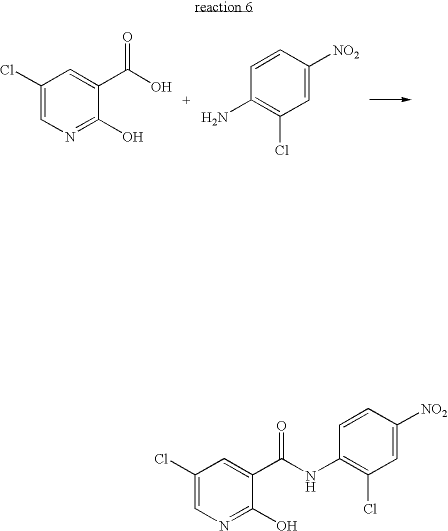

The resulting product is a compound of formula XVIII, and can be used in the methods of the invention.

Functional Analogs of Niclosamide

Based on the shared antihelmenthic activity, compounds such as ivermectin, abamectin, doramectin, moxidectin, mylbemycin D, niclofolan, praziquantel, diamphenethide, and chlorsulon can be substituted for niclosamide in the methods of the invention. Other antihelmenthic agents are known in the art; these compounds can also be employed in the methods of the invention.

Antiproliferative Agents

Antiproliferative agents that can be administered in the combinations of the invention are are described above. Such agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors. Any one or more of the agents listed in Table 1 can be used. Exemplary antiproliferative agents include, without limitation, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine.

Exemplary Drug Combinations

In certain embodiments, the drug combination comprises (1) an antihelminthic agent selected from the group consisting of niclosamide, oxyclozanide, closantel, rafoxanide, resorantel, clioxanide, tribromsalan, dibromsalan, brotianide, 4′-chloro-3-nitrosalicylanilide, 4′-chloro-5-nitrosalicylanilide, 2′-chloro-5′-methoxy-3-nitrosalicylanilide, 2′-methoxy-3,4′-dinitrosalicylanilide, 2′,4′-dimethyl-3-nitrosalicylanilide, 4′,5-dibromo-3-nitrosalicylanilide, 2′-chloro-3,4′-dinitrosalicylanilide, 2′-ethyl-3-nitrosalicylanilide, 2′-bromo-3-nitrosalicylanilide, flusalan, and a salt of the above listed agent and (2) an antiproliferative agent. In certain embodiments, the antiproliferative agent is selected from the group consisting of paclitaxel, gemcitabine, etoposide, irinotecan, and chlorpromazine.

In certain embodiments, the drug combination comprises (1) niclosamide or a salt or ester thereof and (2) an anti-proliferative agent. The niclosamide salt may be ethanolamine, piperazine, or monohydrate salt of niclosamide. In certain embodiments, the antiproliferative agent is selected from the group consisting of paclitaxel, gemcitabine, etoposide, irinotecan, and chlorpromazine.

In certain embodiments, the drug combination comprises (1) an antihelminthic agent selected from the group consisting of ivermectin, abamectin, doramectin, moxidectin, mylbemycin D, niclofolan, praziquantel, diamphenethide, and chlorsulon, and (2) an anti-proliferative agent. In certain embodiments, the antiproliferative agent is selected from the group consisting of paclitaxel, gemcitabine, etoposide, irinotecan, and chlorpromazine.

In other certain embodiments, the antihelminthic agent is selected from ivermectin, abamectin, doramectin, moxidectin, mylbemycin D, niclofolan, praziquantel, diamphenethide, and chlorsulon.

For example, in certain specific embodiments, the drug combination comprises niclosamide and paclitaxel, niclosamide and gemcitabine, niclosamide and etoposide, niclosamide and irinotecan, or niclosamide and chlorpromazine.

Combinations Comprising Chlorpromazine and Pentamidine

In certain embodiments, the drug combinations of the invention may comprise chlorpromazine (or its analogs, salts, or metabolites) and pentamidine (or its analogs, salts, or metabolites). In certain embodiments, the drug combination may further comprise one or more antiproliferative agents (e.g., those listed in Table 1).

Phenothiazines

Phenothiazines that are useful in the antiproliferative combination of the invention are compounds having the general formula (XXIII): 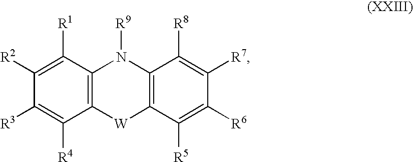
or a pharmaceutically acceptable salt thereof,

wherein R² is selected from the group consisting of: CF₃, halo, OCH₃, COCH₃, CN, OCF₃, COCH₂CH₃, CO(CH₂)₂CH₃, and SCH₂CH₃;

R⁹ has the formula: 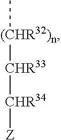
wherein n is 0 or 1, each of R³², R³³, and R³⁴ is, independently, H or substituted or unsubstituted C₁₋₆alkyl, and Z is NR³⁵R³⁶ or OR³⁷, wherein each of R³⁵ and R³⁶ is, independently, H, substituted or unsubstituted C₁₋₆ alkyl, substituted or unsubstituted alkaryl, substituted or unsubstituted alkheteroaryl, and R³⁷ is H, C₁₋₆ alkyl, or C₁₋₇ acyl, wherein any of R³³, R³⁴, R³⁵, and R³⁶ can be optionally taken together with intervening carbon or non-vicinal O, S, or N atoms to form one or more five- to seven-membered rings, substituted with one or more hydrogens, substituted or unsubstituted C₁₋₆ alkyl groups, C₆₋₁₂ aryl groups, alkoxy groups, halogen groups, substituted or unsubstituted alkaryl groups, or substituted or unsubstituted alkheteroaryl groups;

each of R¹, R³, R⁴, R⁵, R⁶, R⁷, and R⁸ is independently H, OH, F, OCF₃, or OCH₃; and W is selected from the group consisting of:

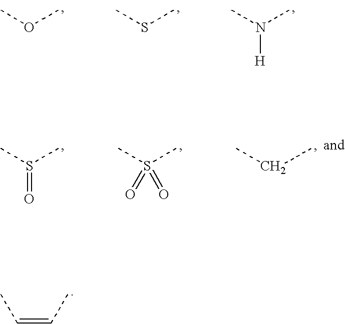

In certain embodiments, R⁹ is selected from the group consisting of:

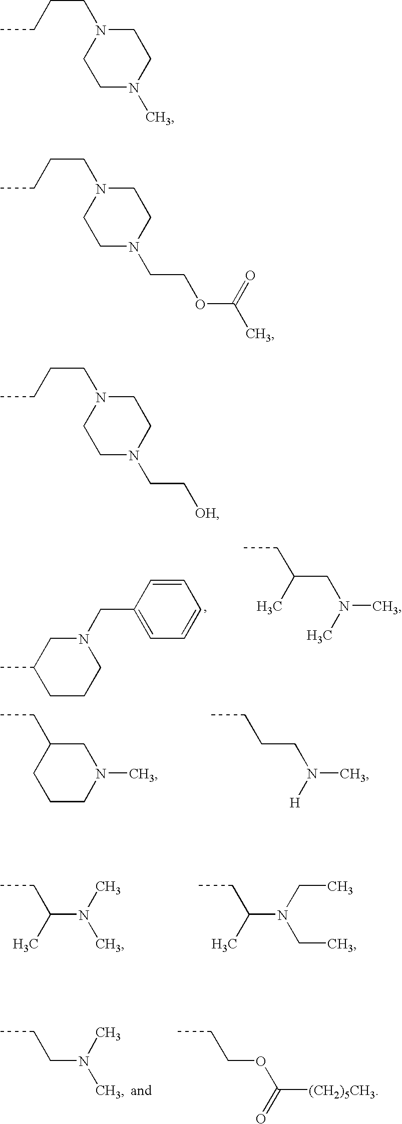

In certain embodiments, wherein R² is selected from the group consisting of: CF₃, halo, OCH₃, COCH₃, CN, OCF₃, COCH₂CH₃, CO(CH₂)₂CH₃, and SCH₂CH₃;

R⁹ is selected from the group consisting of: 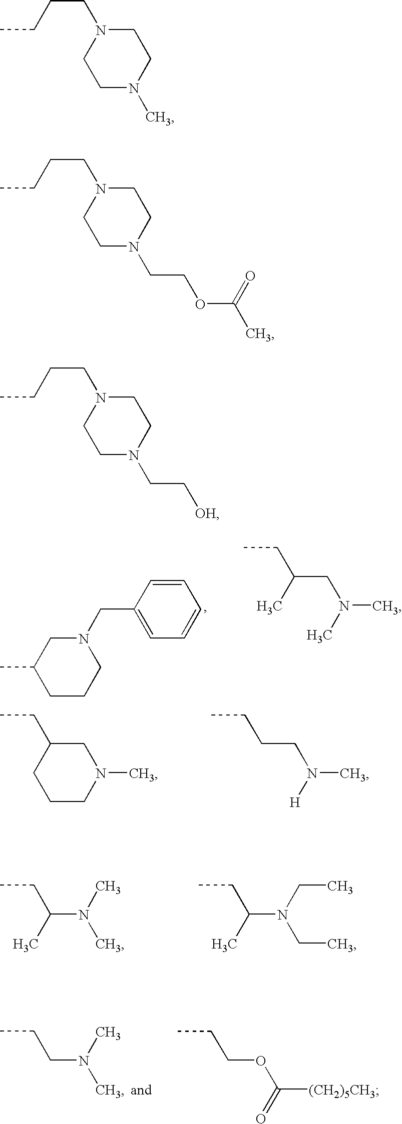
each of R¹, R³, R⁴, R⁵, R⁶, R⁷, and R⁸ is independently H, OH, F, OCF₃, or OCH₃; and W is selected from the group consisting of:

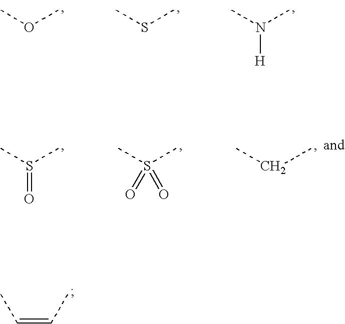

In certain embodiments, R₂ is Cl; each of R₁, R₃, R₄, R₅, R₆, R₇, R₈ is H or F; and R⁹ is selected from the group consisting of:

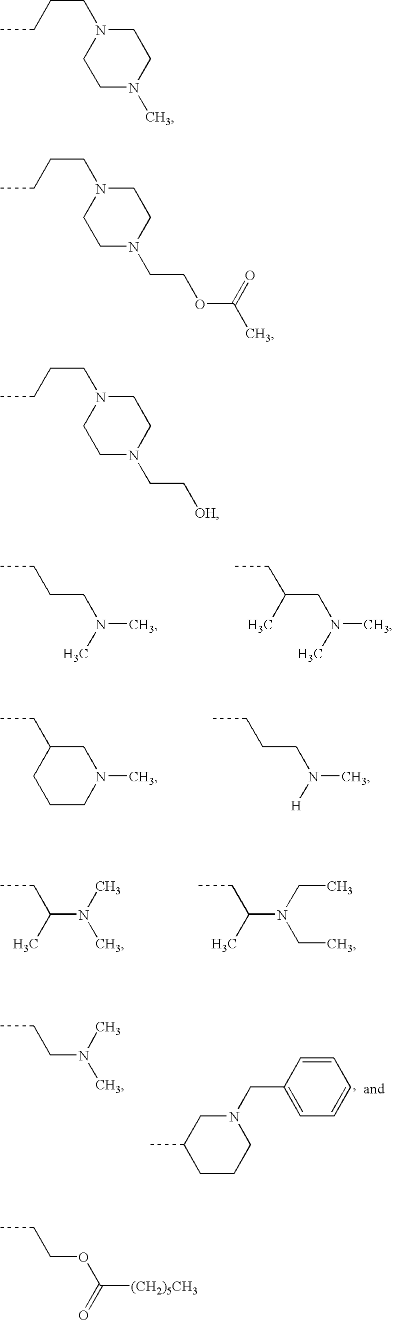

In certain embodiments, R₂, R₃, R₇ and R⁹ are as defined immediately above, and each of R₁, R₄, R₅, R₆, and R₈ is H.

In certain embodiments, the compound of formula (XXIII) is acepromazine, chlorfenethazine, cyamemazine, enanthate, fluphenazine, mepazine, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, or triflupromazine.

In certain other embodiments, the compound of formula (XXIII) is chlorpromazine, perphenazine or promethazine.

Chlorpromazine, Analogs and Metabolites

The most commonly prescribed member of the phenothiazine family is chlorpromazine, which has the structure:

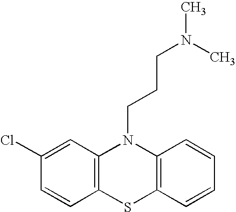

Chlorpromazine is currently available in the following forms: tablets, capsules, suppositories, oral concentrates and syrups, and formulations for injection.

Phenothiazines considered to be chlorpromazine analogs include fluphenazine, prochlorperazine, promethazine, thioridazine, and trifluoperazine. Many of these share antipsychotic or antiemetic activity with chlorpromazine. Also included as chlorpromazine analogs are those compounds in PCT Publication No. WO02/057244, which is hereby incorporated by reference.

Phenothiazines are thought to elicit their antipsychotic and antiemetic effects via interference with central dopaminergic pathways in the mesolimbic and medullary chemoreceptor trigger zone areas of the brain. Extrapyramidal side effects are a result of interactions with dopaminergic pathways in the basal ganglia. Although often termed dopamine blockers, the exact mechanism of dopaminergic interference responsible for the drugs' antipsychotic activity has not been determined.

Phenothiazines are also known to inhibit the activity of protein kinase C. Protein kinase C mediates the effects of a large number of hormones and is involved in may aspects of cellular regulation and carcinogenesis (Castagna, et al., J. Biol. Chem. 1982, 257:7847-51). The enzyme is also thought to play a role in certain types of resistance to cancer chemotherapeutic agents. Chlorpromazine has been investigated for the inhibition of protein kinase C both in vitro (Aftab, et al., Mol. Pharmacology, 1991, 40:798-805) and in vivo (Dwivedi, et al., J. Pharm. Exp. Ther., 1999, 291:688-704). Phenothiazines are also known as calmodulin inhibitors and mitotic kinesin inhibitors, the better of which modulate the movements of spindles and chromosomes in dividing cells.

Chlorpromazine also has strong alpha-adrenergic blocking activity and can cause orthostatic hypotension. Chlorpromazine also has moderate anticholinergic activity manifested as occasional dry mouth, blurred vision, urinary retention, and constipation. Chlorpromazine increases prolactin secretion owing to its dopamine receptor blocking action in the pituitary and hypothalamus.

Because chlorpromazine undergoes extensive metabolic transformation into a number of metabolites that may be therapeutically active, these metabolites may be substituted from chlorpromazine in the antiproliferative combination of the invention. The metabolism of chlorpromazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide.

In other examples of chlorpromazine metabolites useful in the antiproliferative combination of the invention, each of positions 3, 7, and 8 of the phenothiazine can independently be substituted with a hydroxyl or methoxyl moiety.

In certain embodiments, phenothiazines, analogues, derivatives, or metabolites thereof may have a sedative activity.

Pentamidine, Analogs and Metabolites

Pentamidine

Pentamidine is currently used for the treatment of Pneumocystis carinii, Leishmania donovani, Trypanosoma brucei, T gambiense, and T. rhodesiense infections. The structure of pentamidine is:

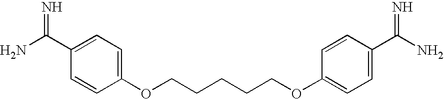

It is available formulated for injection or inhalation. For injection, pentamidine is packaged as a nonpyrogenic, lyophilized product. After reconstitution, it is administered by intramuscular or intravenous injection.

Pentamidine isethionate is a white, crystalline powder soluble in water and glycerin and insoluble in ether, acetone, and chloroform. It is chemically designated 4,4′-diamidino-diphenoxypentane di(β-hydroxyethanesulfonate). The molecular formula is C₂₃H₃₆N₄O₁₀S₂ and the molecular weight is 592.68.

The mode of action of pentamidine is not fully understood. In vitro studies with mammalian tissues and the protozoan Crithidia oncopelti indicate that the drug interferes with nuclear metabolism, producing inhibition of the synthesis of DNA, RNA, phospholipids, and proteins. Several lines of evidence suggest that the action of pentamidine against leishmaniasis, a tropical disease caused by a protozoan residing in host macrophages, might be mediated via host cellular targets and the host immune system. Pentamidine selectively targets intracellular leishmania in macrophages but not the free-living form of the protozoan and has reduced anti-leishmania activity in immunodeficient mice in comparison with its action in immunocompetent hosts.

Recently, pentamidine was shown to be an effective inhibitor of protein tyrosine phosphatase 1B (PTP1B). Because PTP1B dephosphorylates and inactivates Jak kinases, which mediate signaling of cytokines with leishmanicidal activity, its inhibition by pentamidine might result in augmentation of cytokine signaling and anti-leishmania effects. Pentamidine has also been shown to be a potent inhibitor of the oncogenic phosphatases of regenerating liver (such as, for example PRL-1, PRL-2, or PRL-3). Thus, in the methods of the invention, pentamidine can be replaced by any protein tyrosine phosphatase inhibitor, including PTP1B inhibitors or PRL inhibitors. Inhibitors of protein tyrosine phosphatases include levamisole, ketoconazole, bisperoxovanadium compounds (e.g., those described in Scrivens et al., Mol. Cancer Ther. 2:1053-1059, 2003, and U.S. Pat. No. 6,642,221), vandate salts and complexes (e.g., sodium orthovanadate), dephosphatin, dnacin A1, dnacin A2, STI-571, suramin, gallium nitrate, sodium stibogluconate, meglumine antimonate, 2-(2-mercaptoethanol)-3-methyl-1,4-naphthoquinone, 2,5-bis(4-amidinophenyl)furan-bis-O-methylamidoxime, known as DB289 (Immtech), 2,5-bis(4-amidinophenyl)furan (DB75, Immtech), disclosed in U.S. Pat. No. 5,843,980, and compounds described in Pestell et al., Oncogene 19:6607-6612, 2000, Lyon et al., Nat. Rev. Drug Discov. 1:961-976, 2002, Ducruet et al., Bioorg. Med. Chem. 8:1451-1466, 2000, U.S. Patent Application Publication Nos. 2003/0114703, 2003/0144338, 2003/0161893, and PCT Patent Publication Nos. WO99/46237, WO03/06788 and WO03/070158. Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and U.S. Patent Application Publication Nos. US 2001/0044468 and US 2002/0019437, and the pentamidine analogs described in U.S. patent application Ser. No. 10/617,424 (see, e.g., Formula (II)). Other protein tyrosine phosphatase inhibitors can be identified, for example, using the methods described in Lazo et al. (Oncol. Res. 13:347-352, 2003), PCT Publication Nos. WO97/40379, WO03/003001, and WO03/035621, and U.S. Pat. Nos. 5,443,962 and 5,958,719.

Pentamidine has also been shown to inhibit the activity of endo-exonuclease (PCT Publication No. WO 01/35935). Thus, in the methods of the invention, pentamidine can be replaced by any endo-exonuclease inhibitor.

By “endo-exonuclease inhibitor” is meant a compound that inhibits (e.g., by at least 10%, 20%, 30%, or more) the enzymatic activity of an enzyme having endo-exonuclease activity. Such inhibitors include, but are not limited to, pentamidine, pentamidine analogs, and pentamidine metabolites.

By “phosphatase of regenerating liver inhibitor” is meant a compound that inhibits (e.g., by at least 10%, 20%, 30%, or more) the enzymatic activity of a member of the phosphatase of regenerating liver (PRL) family of tyrosine phosphatases. Members of this family include, but are not limited to, PRL-1, PRL-2, and PRL-3. Inhibitors include, but are not limited to, pentamidine, pentamidine analogs, and pentamidine metabolites.

By “protein tyrosine phosphatase 1B inhibitor” is meant a compound that inhibits (e.g., by at least 10%, 20%, 30%, or more) the enzymatic activity of protein phosphatase 1B. Inhibitors include, but are not limited to, pentamidine, pentamidine analogs, and pentamidine metabolites.

Pentamidine Analogs

Aromatic diamidino compounds can replace pentamidine in the antiproliferative combination of the invention. Aromatic diamidino compounds such as propamidine, butamidine, heptamidine, and nonamidine share properties with pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy)phenamidine, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine.

Pentamidine analogs are described, for example, by formula (XXIV) 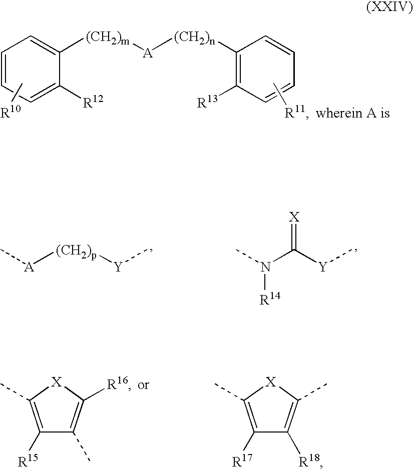
wherein

each of X and Y is, independently, O, NR¹⁹, or S,

each of R¹⁴ and R¹⁹ is, independently, H or C₁-C₆ alkyl,

each of R¹⁵, R¹⁶, R¹⁷, and R¹⁸ is, independently, H, C₁-C₆ alkyl, halogen, C₁-C₆ alkyloxy,C₆-C₁₈ aryloxy, or C₆-C₁₈ aryl-C₁-C₆ alkyloxy,

p is an integer between 2 and 6, inclusive,

each of m and n is, independently, an integer between 0 and 2, inclusive,

each of R¹⁰ and R¹¹ is 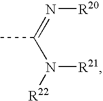
wherein

R²¹ is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R²² is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R²⁰ is H, OH, or C₁-C₆ alkyloxy, or R²⁰ and R²¹ together represent 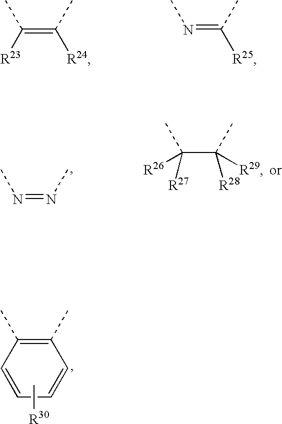
wherein

each of R²³, R²⁴, and R²⁵ is, independently, H, C₁-C₆ alkyl, halogen, or trifluoromethyl, each of R²⁶, R²⁷, R²⁸, and R²⁹ is, independently, H or C₁-C₆ alkyl, and R³⁰ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, each of R¹² and R¹³ is, independently, H, Cl, Br, OH, OCH₃, OCF₃, NO₂, and NH₂, or R¹² and R¹³ together form a single bond.

In certain embodiments, A is

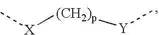

each of X and Y is independently O or NH;

p is an integer between 2 and 6, inclusive; and

m and n are, independently, integers between 0 and 2, inclusive, wherein the sum of m and n is greater than 0.

In certain other embodiments, A is

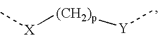

each of X and Y is independently O or NH,

p is an integer between 2 and 6, inclusive,

each of m and n is 0, and

each of R¹⁰ and R¹¹ is, independently, selected from the group represented 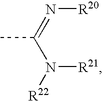
wherein R²¹ is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R²² is H, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl C₁-C₆ alkoxy), carbo(C₆-C₁₈ aryloxy), or C₆-C₁₈ aryl, and R²⁰ is H, OH, or C₁-C₆ alkyloxy, or R²⁰ and R²¹ together represent 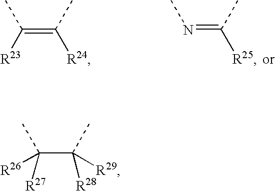
wherein each of R²³, R²⁴, and R²⁵ is, independently, H, C₁-C₆ alkyl, halogen, or trifluoromethyl, each of R²⁶, R²⁷, and R²⁸ is, independently, H or C₁-C₆ alkyl, and R²⁹ is C₁-C₆ alkyl, C₁-C₆ alkyloxy, or trifluoromethyl.

In certain other embodiments, A is

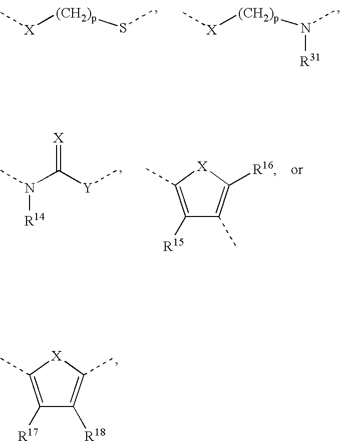

each of X and Y is, independently, O, NR¹⁹, or S,

each of R¹⁴ and R¹⁹ is, independently, H or C₁-C₆ alkyl,

each of R¹⁵, R¹⁶, R¹⁷, and R¹⁸ is, independently, H, C₁-C₆ alkyl, halogen, C₁-C₆ alkyloxy, C₆-C₁₈ aryloxy, or C₆-C₁₈ aryl C₁-C₆ alkyloxy,

R³¹ is C₁-C₆ alkyl,

p is an integer between 2 and 6, inclusive,

each of m and n is, independently, an integer between 0 and 2, inclusive,

each of R¹⁰ and R¹¹ is, independently, selected from the group represented 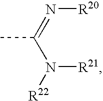
wherein R²¹ is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R²² is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R²⁰ is H, OH, or C₁-C₆ alkyloxy, or R²⁰ and R²¹ together represent

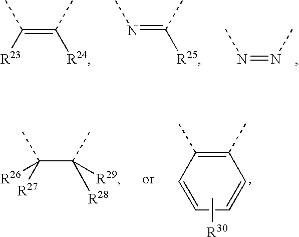

wherein each of R²³, R²⁴, and R²⁵ is, independently, H, C₁-C₆ alkyl, halogen, or trifluoromethyl, each of R²⁶, R²⁷, R²⁸, and R²⁹ are, independently, H or C₁-C₆ alkyl, and R³⁰ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl.

Other analogs include stilbamidine (G-1) and hydroxystilbamidine (G-2), and their indole analogs (e.g., G-3).

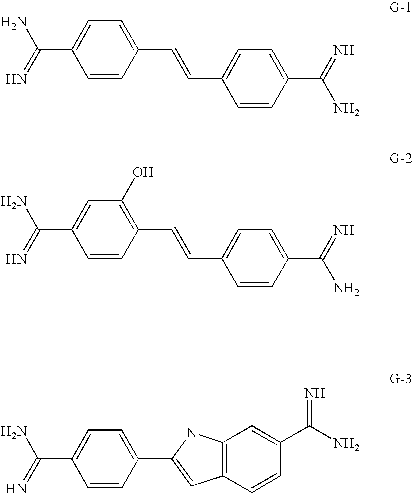

Each amidine moiety in G-1, G-2, or G-3 may be replaced with one of the moieties depicted in formula (XXIV) above as

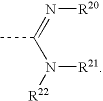

As is the case for pentamidine, salts of stilbamidine and its related compounds are also useful in the method of the invention. Preferred salts include, for example, dihydrochloride and methanesulfonate salts.

Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1, each of which is in its entirety incorporated by reference.

Exemplary analogs are 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1, 4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis [5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazol]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N, N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan. Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

In certain embodiments, the compound of formula (XXIV) is propamidine, butamidine, heptamidine, nonamidine, stilbamidine, hydroxystilbamidine, diminazene, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, or 2,4-bis(4-amidinophenyl)thiophene-bis-O-methylamidoxime.

In certain embodiment, the compound of formula (XXIV) is pentamidine, 2,5-bis(4-amidinophenyl)furan, or 2,5-bis(4-amidinophenyl)furan-bis-O-methylamidoxime.

In certain embodiments, the second compound of drug combinations can be a functional analog of pentamidine, such as netropsin, distamycin, bleomycin, actinomycin, daunorubicin, or a compound that falls within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1.

Pentamidine Metabolites

Pentamidine metabolites are also useful in the antiproliferative combination of the invention. Pentamidine is rapidly metabolized in the body to at least seven primary metabolites. Some of these metabolites share one or more activities with pentamidine. It is likely that some pentamidine metabolites will have anti-cancer activity when administered in combination with an antiproliferative agent. Seven pentamidine metabolites (H-1 through H-7) are shown below.

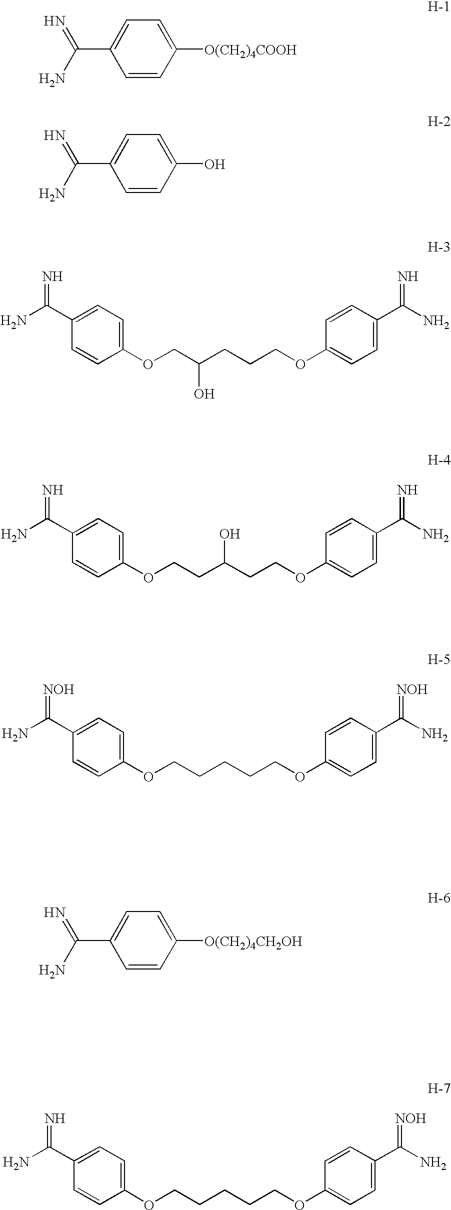

In certain embodiments, pentamidine, or its analog, derivative, or metabolite may have an antibiotic activity.

Antiproliferative Agents

In certain embodiments, an antiproliferative agent may be further included in the drug combinations that comprise (1)pentamidine (or its analog) and (2) chlorpromazine or its analogue). Antiproliferative agents are described above. Such agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors. In certain embodiments, the antiproliferative agent is a Group A antiproliferative agent as described below in the section describing combinations comprising pentamidine and antiproliferative agents (e.g., an agent listed in Table 1).

Exemplary Drug Combinations

In certain embodiments, the drug combinations of the present invention may comprise (a) a first compound selected from the group consisting of prochlorperazine, perphenazine, mepazine, methotrimeprazine, acepromazine, thiopropazate, perazine, propiomazine, putaperazine, thiethylperazine, methopromazine, chlorfenethazine, cyamemazine, perphenazine, norchlorpromazine, trifluoperazine, thioridazine (or a salt of any of the above), and dopamine D2 antagonists (e.g., sulpride, pimozide, spiperone, ethopropazine, clebopride, bupropion, and haloperidol), and (b) a second compound selected from the group consisting of pentamidine, propamidine, butamidine, heptamidine, nonamidine, stilbamidine, hydroxystilbamidine, diminazene, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H -benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹ -trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a salt of any of the above.

In certain embodiments, drug combinations may comprise (1) a first compound selected from the group consisting of acepromazine, chlorfenethazine, cyamemazine, enanthate, fluphenazine, mepazine, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, triflupromazine, and a pharmaceutically active or acceptable salt thereof, and (2) a second compound selected from the group consisting of propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, or a pharmaceutically acceptable salt thereof.

In certain embodiments, drug combinations may comprise (1) a first compound selected from the group consisting of chlorpromazine, perphenazine or promethazine, and a pharmaceutically active or acceptable salt thereof, and (2) a second compound selected from the group consisting of pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, or a pharmaceutically acceptable salt thereof.

In certain embodiments, the drug combination comprises (1) a compound of formula (XXIII) selected from chlorpromazine, perphenazine or promethazine and (2) a compound of formula (XXIV) selected from pentamidine, 2,5-bis(4-amidinophenyl)furan, or 2,5-bis(4-amidinophenyl)furan-bis-O-methylamidoxime.

In certain embodiments, drug combinations may comprise (1) an inhibitor of protein kinase C, and (2) a compound of formula (XXIV).

In certain embodiments, drug combinations may comprise (1) a compound of formula (XXIII), and (2) an endo-exonuclease inhibitor.

In certain embodiments, drug combinations may comprise (1) a compound of formula (XXIII), and (2) a PRL phosphatase inhibitor or a PTP1B inhibitor.

In certain embodiments, drug combinations may comprise chlorpromazine and pentamidine.

Combinations Comprising Benzimidazoles and Antiprotozoal Drugs

In certain embodiments, the drug combinations according to the present invention may comprise a benzimidazole (e.g., albendazole, mebendazole, and oxibendazole, including their structural or function analogs, salts and metabolites) and an antiprotozoal drug. In certain other embodiments, the above drug combinations may further comprise one or more antiproliferative agents (e.g., those in Table 1).

In certain embodiments, the drug combinations according to the present invention may comprise benzimidazole (e.g., albendazole, mebendazole, and oxibendazole, including their structural or function analog and metabolites) and an antiproliferative agent.

In certain embodiments, the drug combinations according to the present invention may comprise an antiprotozoal drug and an antiproliferative agent.

Benzimidazoles

Benzimidazoles that are useful in the antiproliferative combination of the invention include compounds having the general formula (XXV): 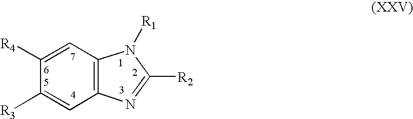
wherein:

R¹ is selected from the group consisting of H and C₁₋₁₀ alkyl or C₂₋₁₀ alkenyl that is unsubstituted or substituted by one or more substituents selected from the group consisting of aryl, heteroaryl, heterocyclyl, OC₁₋₁₀ alkyl, O(C₁₋₁₀)₀₋₁-aryl, O-(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂-C₁₋₁₀ alkyl, S(O)₀₋₂—(C₁₋₁₀ alkyl)0-1-aryl, S(O)₀₋₂-(C₁₋₁₀ alkyl)₀₋₁-heteroaryl, S(O)₀₋₂—(C₁₋₁₀ alkyl)₀₋₁-heterocyclyl, N(R₁₃)₂, OR₁₃, oxo, cyano, halo, NO₂, OH, and SH; R₂ is selected from the group consisting of:

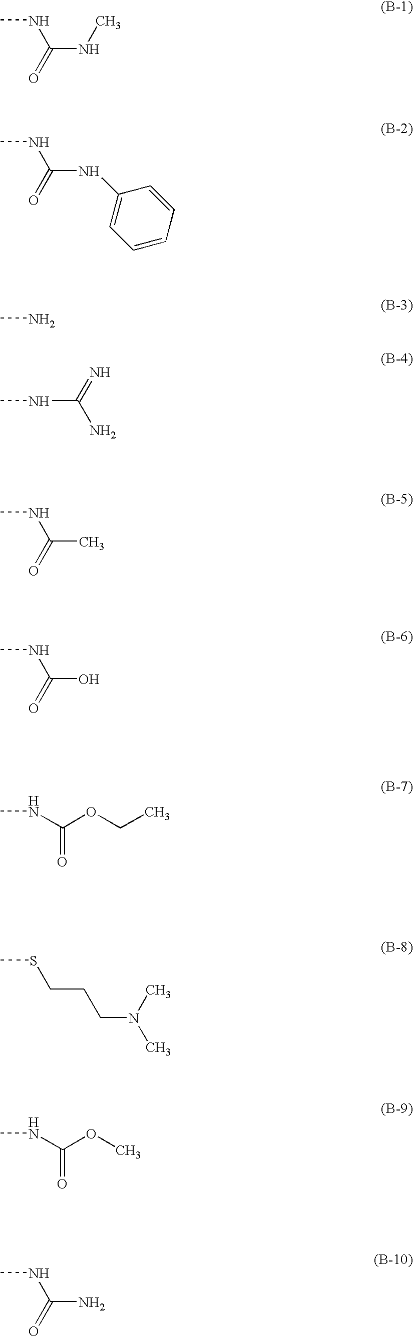

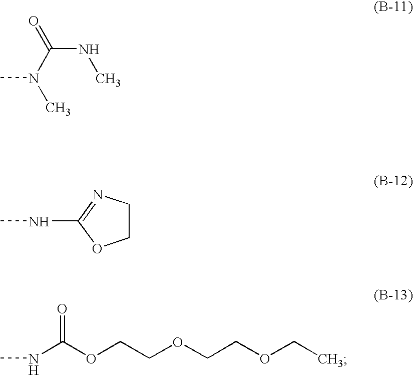

each of R₃ and R₄ is independently selected from the group consisting of H, halo, NO₂, OH, SH, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂-(C₁₋₁₀ alkyl)₀₋₁-heterocyclyl, and C₁₋₁₀ alkyl or C₂₋₁₀ alkenyl that is unsubstituted or substituted by one or more substituents selected from the group consisting of aryl, heteroaryl, heterocyclyl, O—C₁₋₁₀-alkyl, O(C₁₋₁₀ alkyl)₀₋₁₋-aryl, O(₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁₀-aryl, S(O)₀₋₂—(C₁₋₁₀ alkyl)₀₋₁-heteroaryl, S(O)₀₋₂-(C₁₋₁₀ alkyl)₀₋₁-heterocyclyl, N(R₁₃)₂, OR₁₃, oxo, cyano, halogen, NO₂, OH, and SH; and each R₁₃ is selected from the group consisting of H and C₁₋₁₀ alkyl or C₂₋₁₀ alkenyl that is unsubstituted or substituted by one or more substituents selected from the group consisting of aryl, heteroaryl, heterocyclyl, O—C₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C-₁₋₁₀ alkoxycarbonyl, oxo, cyano, halo, NO₂, OH, and SH.

Examples of substituents R₁, R₃, and R₄ are provided below. 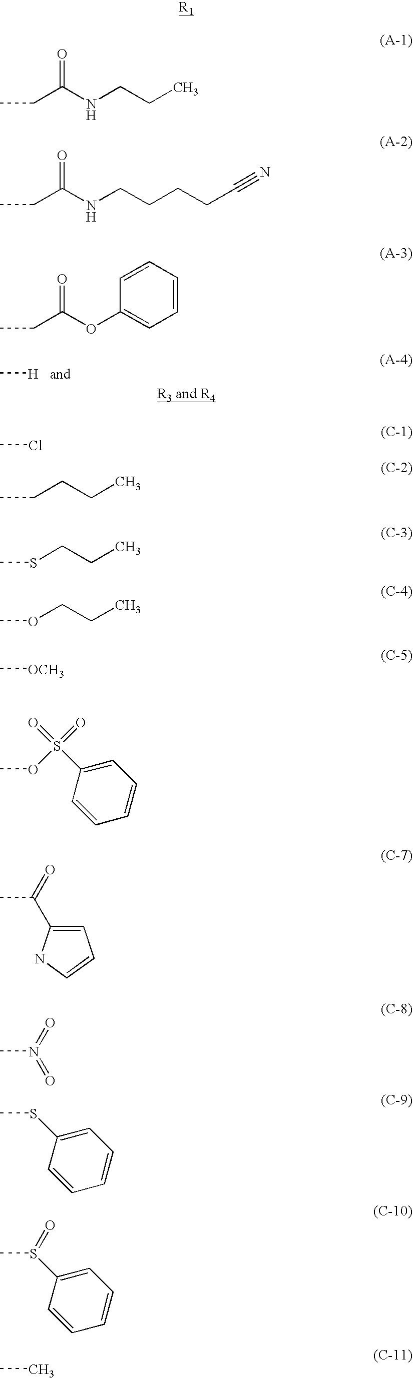 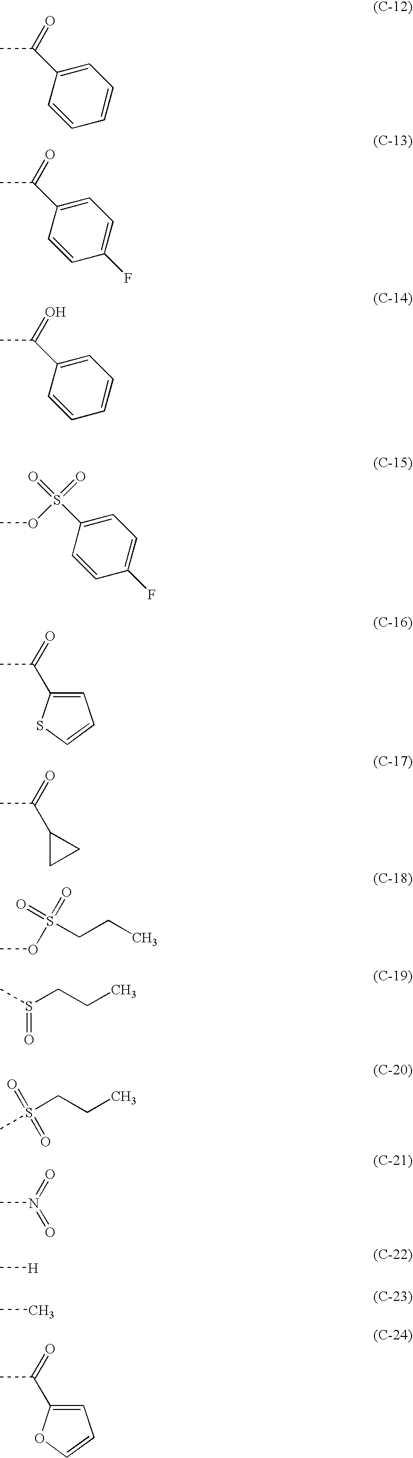 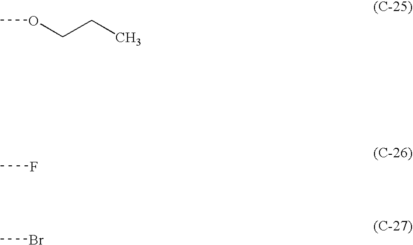
Albendazole

One of the most commonly prescribed members of the benzimidazole family is albendazole, which has the structure:

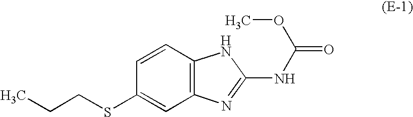

Albendazole is currently available as an oral suspension and in tablets.

Albendazole Metabolites

Albendazole undergoes metabolic transformation into a number of metabolites that may be therapeutically active; these metabolites may be substituted for albendazole in the antiproliferative combination of the invention. The metabolism of albendazole can yield, for example, albendazole sulfonate, albendazole sulfone, and albendazole sulfoxide.

Benzimidazole Analogs

Analogs of benzimidazoles include benzothioles and benzoxazoles having the structure of formula (XXVI): 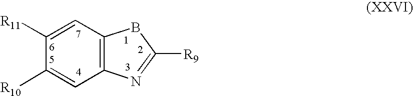
wherein: B is O or S; R₉ is selected from the group consisting of:

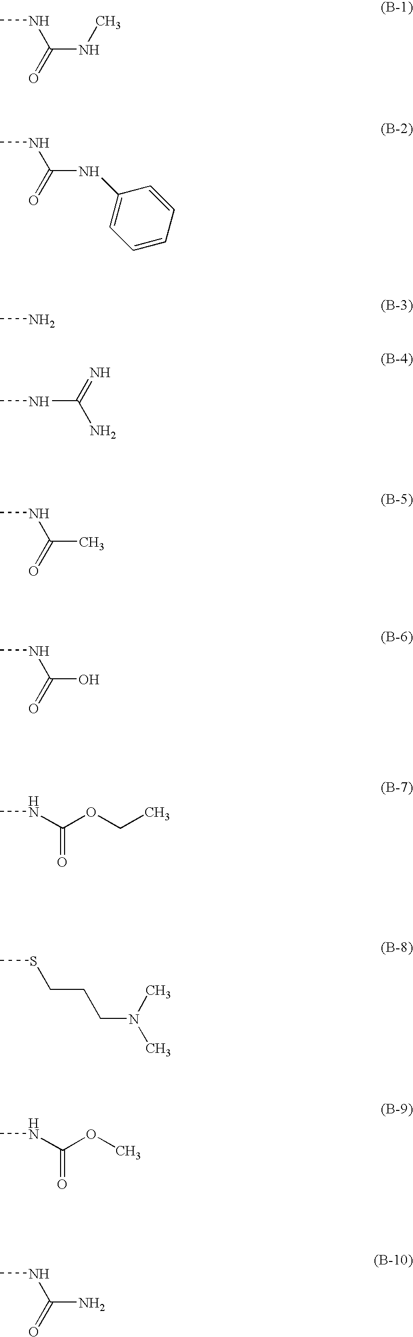

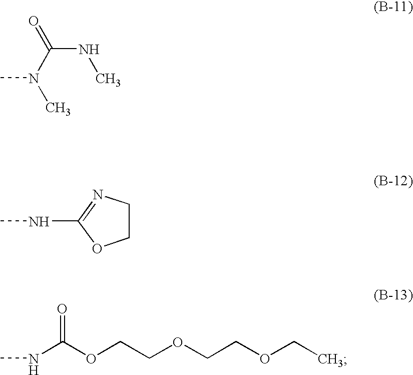

and each of R₁₀ and R₁₁ is independently selected from the group consisting of H,

halo, NO₂, OH, SH, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂-C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, and C₁₋₁₀ alkyl or C₂₋₁₀ alkenyl that is unsubstituted or substituted by one or more substituents selected from the group consisting of aryl, heteroaryl, heterocyclyl, OC₁₋₁₀ alkyl, O(C₁₋₁₀ alkyl)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂-C₁₋₁₀ alkyl, S(O)₀₋₂-(C₁₋₁₀ alkyl)₀₋₁-aryl, S(O)₀₋₂—(C₁₋₁₀ alkyl)₀₋₁-heteroaryl, S(O)₀₋₂-(C₁₋₁₀ alkyl)₀₋₁-heterocyclyl, N(R₁₃)₂, OR₁₃, oxo, cyano, halo, NO₂, OH, and SH; and each R₁₃ is independently selected from the group consisting of H and C₁₋₁₀ alkyl or C₂₋₁₀ alkenyl that is unsubstituted or substituted by one or more substituents selected from the group consisting of aryl, heteroaryl, heterocyclyl, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, oxo, cyano, halo, NO₂, OH, and SH.

Some benzimidazoles and benzimidazole analogs fit the following formula (XXVII).

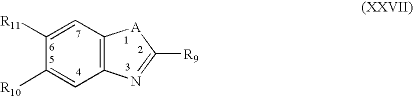

wherein A is selected from the group consisting of O, S, and NR₁₂; R₉ R₁₀, R₁₁, and R₁₃ are as described above for formula (XXVI); and R₁₂ is selected from the group consisting of H and C₁₋₁₀ alkyl or C₂₋₁₀ alkenyl that is unsubstituted or substituted by one or more substituents selected from the group consisting of aryl, heteroaryl, heterocyclyl, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂-C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, N(R₁₃)₂, OR₁₃, oxo, cyano, halo, NO₂, OH, and SH.

Exemplary Benzimidazoles and their Analogs

In certain embodiments, benzimidales or its analogs useful in the present invention may be selected from the group consisting of a first compound selected from albendazole; albendazole sulfonate; albendazole sulfone; albendazole sulfoxide; astemizole; benomyl; 2-benzimidazolylurea; benzthiazuron; cambendazole; cyclobendazole; domperidone; droperidol; fenbendazole; flubendazole; frentizole; 5-hydroxymebendazole; lobendazole; luxabendazole; mebendazole; methabenzthiazuron; mercazole; midefradil; nocodozole; omeprazole; oxfendazole; oxibendazole; parbendazole; pimozide; and tioxidazole (or a salt of any of the above); NSC 181928 (ethyl 5-amino-1,2dihydro-3-[(N-methylanilino)methyl]-pyrido[3,4-b]pyrazin-7-ylcarbamate); TN-16 (3-(1-anilinoethylidene)-5-benzyl-pyrrodiline-2,4-dione); and pharmaceutically active or acceptable salts thereof.

It will be understood by those in the art that the compounds are also useful when formulated as salts. For example, benzimidazole salts include halide, sulfate, nitrate, phosphate, and phosphinate salts.

Pentamidine and Its Analogs

Pentamidine

Pentamidine is described in detail above.

Pentamidine Analogs

Aromatic diamidino compounds can replace pentamidine in the antiproliferative combination of the invention. These compounds are referred to as pentamidine analogs. Examples are propamidine, butamidine, heptamidine, and nonamidine, all of which, like pentamidine, exhibit antipathogenic or DNA binding properties. Other analogs (e.g., stilbamidine and indole analogs of stilbamidine, hydroxystilbamidine, diminazene, benzamidine, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties in common with pentamidine.

Suitable analogs include those falling within formula (XXVIII). 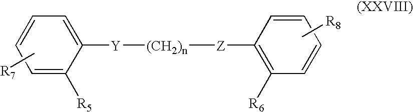
wherein each of Y and Z is, independently, O or N; each of R₅ and R₆ is, independently, H, OH, Cl, Br, F, OCH₃, OCF₃, NO₂, or NH₂; n is an integer between 2 and 6, inclusive; and each of R₇ and R₈ is, independently, at the meta or para position and is selected from the group consisting of:

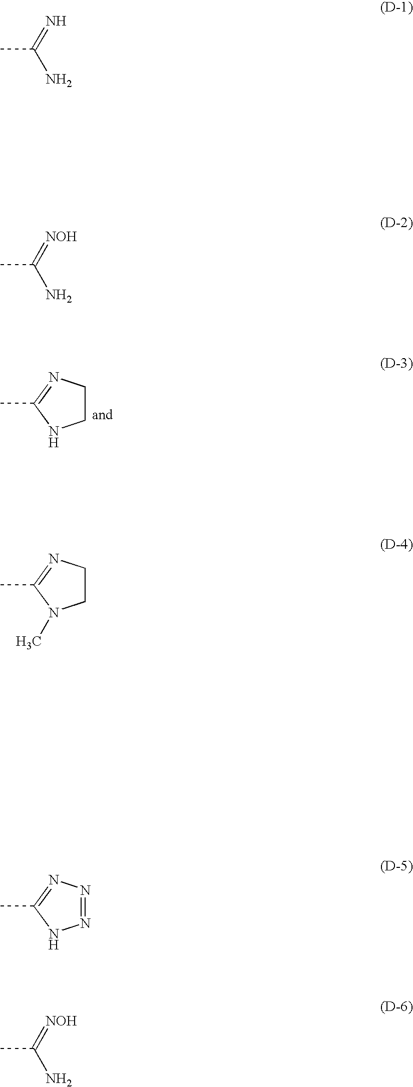

Other suitable pentamidine analogs include stilbamidine (G-1) and hydroxystilbamidine (G-2), and their indole analogs (e.g., G-3):

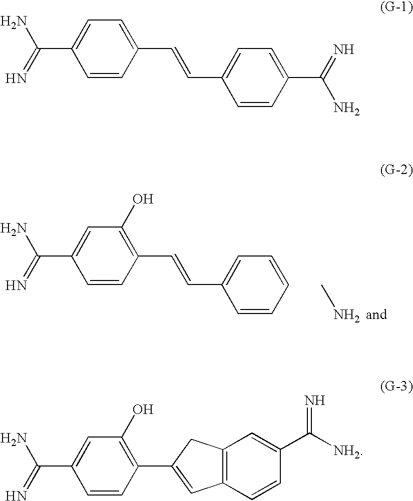

Each amidine moiety may independently be replaced with one of the moieties depicted as D-2, D-3, D-4, D-5, or D-6 above. As is the case for the benzimidazoles and pentamidine, salts of stilbamidine, hydroxystilbamidine, and their indole derivatives are also useful in the method of the invention. Preferred salts include, for example, dihydrochloride and methanesulfonate salts.

Still other analogs are those that fall within a formula provided in any of U.S. Pat. Nos. 5,428,051; 5,521,189; 5,602,172; 5,643,935; 5,723,495; 5,843,980; 6,172,104; and 6,326,395, or U.S. Patent Application Publication No. US 2002/0019437 A1, each of which is in its entirety incorporated by reference. Exemplary analogs include 1,5-bis-(4′-(N-hydroxyamidino)phenoxy)pentane; 1,3-bis-(4′-(N-hydroxyamidino)phenoxy)propane; 1,3-bis-(2′-methoxy-4′-(N-hydroxyamidino)phenoxy)propane; 1,4-bis-(4′-(N-hydroxyamidino)phenoxy)butane; 1,5-bis-(4′-(N-hydroxyamidino)phenoxy)pentane; 1,4-bis-(4′-(N-hydroxyamidino)phenoxy)butane; 1,3-bis-(4′-(4-hydroxyamidino)phenoxy)propane; 1,3-bis-(2′-methoxy-4′-(N-hydroxyamidino)phenoxy)propane; 2,5-bis-[4-amidinophenyl]furan; 2,5-bis-[4-amidinophenyl]furan bis-amidoxime; 2,5-bis-[4-amidinophenyl]furan bis-O-methylamidoxime; 2,5-bis-[4-amidinophenyl]furan bis-O-ethylamidoxime; 2,8-diamidinodibenzothiophene; 2,8-bis-(N-isopropylamidino)carbazole; 2,8-bis-(N-hydroxyamidino)carbazole; 2,8-bis-(2-imidazolinyl)dibenzothiophene; 2,8-bis-(2-imidazolinyl)-5,5-dioxodibenzothiophene; 3,7-diamidinodibenzothiophene; 3,7-bis-(N-isopropylamidino)dibenzothiophene; 3,7-bis-(N-hydroxyamidino)dibenzothiophene; 3,7-diaminodibenzothiophene; 3,7-dibromodibenzothiophene; 3,7-dicyanodibenzothiophene; 2,8-diamidinodibenzofuran; 2,8-di(2-imidazolinyl)dibenzofuran; 2,8-di(N-isopropylamidino)dibenzofuran; 2,8-di(N-hydroxylamidino)dibenzofuran; 3,7-di(2-imidazolinyl)dibenzofuran; 3,7-di(isopropylamidino)dibenzofuran; 3,7-di(A-hydroxylamidino)dibenzofuran; 2,8-dicyanodibenzofuran; 4,4′-dibromo-2,2′-dinitrobiphenyl; 2-methoxy-2′-nitro-4,4′-dibromobiphenyl; 2-methoxy-2′-amino-4,4′-dibromobiphenyl; 3,7-dibromo-dibenzofuran; 3,7-dicyano-dibenzofuran; 2,5-bis-(5-amidino-2-benzimidazolyl)pyrrole; 2,5-bis-[5-(2-imidazolinyl)-2-benzimidazolyl]pyrrole; 2,6-bis-[5-(2-imidazolinyl)-2-benzimidazolyl]pyridine; 1-methyl-2,5-bis-(5-amidino-2-benzimidazolyl)pyrrole; 1-methyl-2,5-bis-[5-(2-imidazolyl)-2-benzimidazolyl]pyrrole; 1-methyl-2,5-bis-[5-(1,4,5,6-tetrahydro-2-pyrimidinyl)-2-benzimidazolyl]pyrrole; 2,6-bis-(5-amidino-2-benzimidazoyl)pyridine; 2,6-bis-[5-(1,4,5,6-tetrahydro-2-pyrimidinyl)-2-benzimidazolyl]pyridine; 2,5-bis-(5-amidino-2-benzimidazolyl)furan; 2,5-bis-[5-(2-imidazolinyl)-2-benzimidazolyl]furan; 2,5-bis-(5-N-isopropylamidino-2-benzimidazolyl)furan; 2,5-bis-(4-guanylphenyl)furan; 2,5-bis(4-guanylphenyl)-3,4-dimethylfuran; 2,5-di-p[2(3,4,5,6-tetrahydropyrimidyl)phenyl]furan; 2,5-bis-[4-(2-imidazolinyl)phenyl]furan; 2,5-[bis-{4-(2-tetrahydropyrimidinyl)}phenyl]-p(tolyloxy)furan; 2,5-[bis{4-(2-imidazolinyl)}phenyl]3-p(tolyloxy)furan; 2,5-bis-{4-[5-(N-2-aminoethylamido)benzimidazol-2-yl]phenyl}furan; 2,5-bis[4-(3a,4,5,6,7,7a-hexahydro-1H-benzimidazol-2-yl)phenyl]furan; 2,5-bis-[4-(4,5,6,7-tetrahydro-1H-1,3-diazepin-2-yl)phenyl]furan; 2,5-bis-(4-N,N-dimethylcarboxhydrazidephenyl)furan; 2,5-bis-{4-[2-(N-2-hydroxyethyl)imidazolinyl]-phenyl}furan; 2,5-bis[4-(N-isopropylamidino)phenyl]furan; 2,5-bis-{4-[3-(dimethylaminopropyl)amidino]phenyl}furan; 2,5-bis-{4-[N-(3-aminopropyl)amidino]phenyl}furan; 2,5-bis-[2-(imidzaolinyl)phenyl]-3,4-bis(methoxymethyl)furan; 2,5-bis-[4-N-(dimethylaminoethyl)guanyl]phenylfuran; 2,5-bis-{4-[(N-2-hydroxyethyl)guanyl]phenyl}furan; 2,5-bis-[4-N-(cyclopropylguanyl)phenyl]furan; 2,5-bis-[4-(N,N-diethylaminopropyl)guanyl]phenylfuran; 2,5-bis-{4-[2-(N-ethylimidazolinyl)]phenyl}furan; 2,5-bis-{4-[N-(3-pentylguanyl)]}phenylfuran; 2,5-bis-[4-(2-imidazolinyl)phenyl]-3-methoxyfuran; 2,5-bis-[4-(N-isopropylamidino)phenyl]-3-methylfuran; bis-[5-amidino-2-benzimidazolyl]methane; bis-[5-(2-imidazolyl)-2-benzimidazolyl]methane; 1,2-bis-[5-amidino-2-benzimidazolyl]ethane; 1,2-bis-[5-(2-imidazolyl)-2-benzimidazolyl]ethane; 1,3-bis-[5-amidino-2-benzimidazolyl]propane; 1,3-bis-[5-(2-imidazolyl)-2-benzimidazolyl]propane; 1,4-bis-[5-amidino-2-benzimidazolyl]propane; 1,4-bis-[5-(2-imidazolyl)-2-benzimidazolyl]butane; 1,8-bis-[5-amidino-2-benzimidazolyl]octane; trans-1,2-bis-[5-amidino-2-benzimidazolyl]ethene; 1,4-bis-[5-(2-imidazolyl)-2-benzimidazolyl]1-butene; 1,4-bis-[5-(2-imidazolyl)-2-benzimidazolyl]2-butene; 1,4-bis-[5-(2-imidazolyl)-2-benzimidazolyl]1-methylbutane; 1,4-bis-[5-(2-imidazolyl)-2-benzimidazolyl]2-ethylbutane; 1,4-bis-[5-(2-imidazolyl)-2-benzimidazolyl]1-methyl-1-butene; 1,4-bis-[5-(2-imidazolyl)-2-benzimidazolyl]2,3-diethyl-2-butene; 1,4-bis-[5-(2-imidazolyl)-2-benzimidazolyl]1,3-butadiene; 1,4-bis-[5-(2-imidazolyl)-2-benzimidazolyl]2-methyl-1,3-butadiene; bis-[5-(2-pyrimidyl)-2-benzimidazolyl]methane; 1,2-bis-[5-(2-pyrimidyl)-2-benzimidazolyl]ethane; 1,3-bis-[5-amidino-2-benzimidazolyl]propane; 1,3-bis-[5-(2-pyrimidyl)-2-benzimidazolyl]propane; 1,4-bis-[5-(2-pyrimidyl)-2-benzimidazolyl]butane; 1,4-bis-[5-(2-pyrimidyl)-2-benzimidazol 1-butene; 1,4-bis-[5-(2-pyrimidyl)-2-benzimidazolyl]2-butene; 1,4-bis-[5-(2-pyrimidyl)-2-benzimidazolyl]1-methylbutane; 1,4-bis-[5-(2-pyrimidyl)-2-benzimidazolyl]2-ethylbutane; 1,4-bis-[5-(2-pyrimidyl)-2-benzimidazolyl]1-methyl-1-butene; 1,4-bis-[5-(2-pyrimidyl)-2-benzimidazolyl]2,3-diethyl-2-butene; 1,4-bis-[5-(2-pyrimidyl)-2-benzimidazolyl]1,3-butadiene; and 1,4-bis-[5-(2-pyrimidyl)-2-benzimidazolyl]2-methyl-1,3-butadiene; 2,4-bis-(4-guanylphenyl)-pyrimidine; 2,4-bis-(4-imidazolin-2-yl)-pyrimidine; 2,4-bis-[(tetrahydropyrimidinyl-2-yl)phenyl]pyrimidine; 2-(4-[N-i-propylguanyl]phenyl)-4-(2-methoxy-4-[N-i-propylguanyl]phenyl)pyrimidine; 4-(N-cyclopentylamidino)-1,2-phenylene diamine; 2,5-bis-[2-(5-amidino)benzimidazoyl]furan; 2,5-bis-[2-{5-(2-imidazolino)}benzimidazoyl]furan; 2,5-bis-[2-(5-N-isopropylamidino) benzimidazoyl]furan; 2,5-bis-[2-(5-N-cyclopentylamidino)benzimidazoyl]furan; 2,5-bis[2-(5-amidino)benzimidazoyl]pyrrole; 2,5-bis-[2-{5-(2-imidazolino)}benzimidazoyl]pyrrole; 2,5-bis[2-(5-N-isopropylamidino)benzimidazoyl]pyrrole; 2,5-bis-[2-(5-N-cyclopentylamidino)benzimidazoyl]pyrrole; 1-methyl-2,5-bis-[2-(5-amidino)benzimidazoyl]pyrrole; 2,5-bis-[2-{5-(2-imidazolino)}benzimidazoyl]-1-methylpyrrole; 2,5-bis[2-(5-N-cyclopentylamidino)benzimidazoyl]1-methylpyrrole; 2,5-bis-[2-(5-N-isopropylamidino)benzimidazoyl]thiophene; 2,6-bis-[2-{5-(2-imidazolino)}benzimidazoyl]pyridine; 2,6-bis-[2-(5-amidino)benzimidazoyl]pyridine; 4,4′-bis-[2-(5-N-isopropylamidino)benzimidazoyl]1,2-diphenylethane; 4,4′-bis-[2-(5-N-cyclopentylamidino)benzimidazoyl]-2,5-diphenylfuran; 2,5-bis-[2-(5-amidino) benzimidazoyl]benzo[b]furan; 2,5-bis-[2-(5-N-cyclopentylamidino)benzimidazoyl]benzo[b]furan; 2,7-bis-[2-(5-N-isopropylamidino)benzimidazoyl]fluorine; 2,5-bis-[4-(3-(N-morpholinopropyl)carbamoyl)phenyl]furan; 2,5-bis-[4-(2-N,N-dimethylaminoethylcarbamoyl)phenyl]furan; 2,5-bis-[4-(3-N,N-dimethylaminopropylcarbamoyl)phenyl]furan; 2,5-bis-[4-(3-N-methyl-3-N-phenylaminopropylcarbamoyl)phenyl]furan; 2,5-bis-[4-(3-N,N⁸,N¹¹-trimethylaminopropylcarbamoyl)phenyl]furan; 2,5-bis-[3-amidinophenyl]furan; 2,5-bis-[3-(N-isopropylamidino)amidinophenyl]furan; 2,5-bis[3[(N-(2-dimethylaminoethyl)amidino]phenylfuran; 2,5-bis-[4-(N-2,2,2-trichloroethoxycarbonyl)amidinophenyl]furan; 2,5-bis-[4-(N-thioethylcarbonyl) amidinophenyl]furan; 2,5-bis-[4-(N-benzyloxycarbonyl)amidinophenyl]furan; 2,5-bis[4-(N-phenoxycarbonyl)amidinophenyl)furan; 2,5-bis-[4-(N-(4-fluoro)-phenoxycarbonyl)amidinophenyl]furan; 2,5-bis-[4-(N-(4-methoxy)phenoxycarbonyl)amidinophenyl]furan; 2,5-bis-[4(1-acetoxyethoxycarbonyl) amidinophenyl]furan; and 2,5-bis-[4-(N-(3-fluoro)phenoxycarbonyl)amidinophenyl]furan. Methods for making any of the foregoing compounds are described in U.S. Pat. Nos. 5,428,051; 5,521,189; 5,602,172; 5,643,935; 5,723,495; 5,843,980; 6,172,104; and 6,326,395, or U.S. Patent Application Publication No. US 2002/0019437 A1.

Pentamidine Metabolites

Pentamidine metabolites are also useful in the antiproliferative combination of the invention. Pentamidine is rapidly metabolized in the body to at least seven primary metabolites. Some of these metabolites share one or more activities with pentamidine. It is likely that some pentamidine metabolites will exhibit antiproliferative activity when combined with a benzimidazole or an analog thereof.

Seven pentamidine metabolites are shown below.

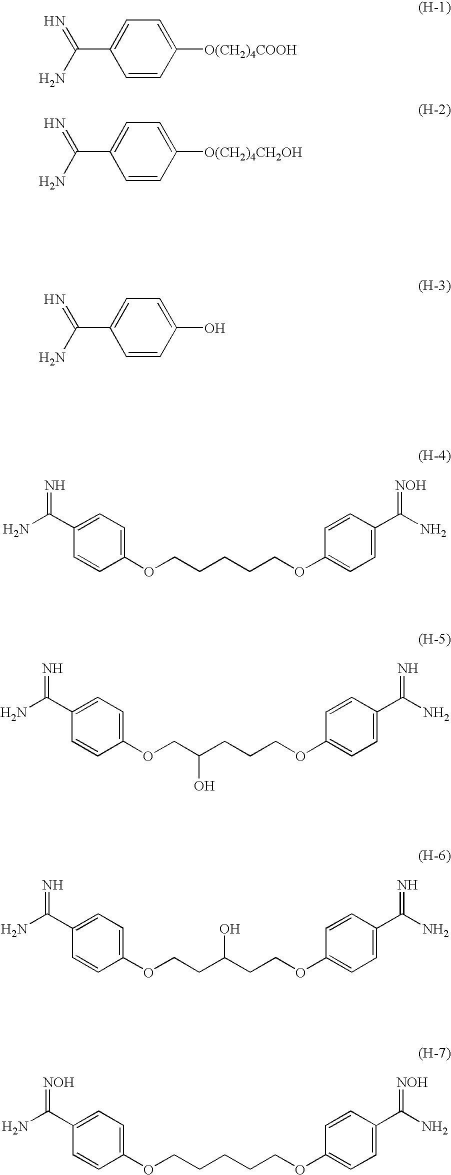

It will be understood by those in the art that the compounds are also useful when formulated as salts. For example, pentamidine salts include the isethionate salt, the platinum salt, the dihydrochloride salt, and the dimethanesulfonate salt (see, for example, Mongiardo et al., Lancet 2:108, 1989).

Exemplary Drug Combinations

In certain embodiments, the drug combinations according to the present invention may comprises (a) a first compound selected from albendazole; albendazole sulfonate; albendazole sulfone; albendazole sulfoxide; astemizole; benomyl; 2-benzimidazolylurea; benzthiazuron; cambendazole; cyclobendazole; domperidone; droperidol; fenbendazole; flubendazole; frentizole; 5-hydroxymebendazole; lobendazole; luxabendazole; mebendazole; methabenzthiazuron; mercazole; midefradil; nocodozole; omeprazole; oxfendazole; oxibendazole; parbendazole; pimozide; and tioxidazole (or a salt of any of the above); NSC 181928 (ethyl 5-amino-1,2-dihydro-3-[(N-methylanilino)methyl]-pyrido[3,4-b]pyrazin-7-ylcarbamate); and TN-16 (3-(1-anilinoethylidene)-5-benzyl-pyrrodiline-2,4-dione); and (b) a second compound selected from pentamidine; propamidine; butamidine; heptamidine; nonamidine; stilbamidine; hydroxystilbamidine; diminazene; benzamidine; phenamidine; dibrompropamidine; 1,3-bis-(4-amidino-2-methoxyphenoxy)propane; phenamidine; amicarbalide; 1,5-bis-(4′-(N-hydroxyamidino)phenoxy)pentane; 1,3-bis-(4′-(N-hydroxyamidino)phenoxy)propane; 1,3-bis-(2′-methoxy-4′-(N-hydroxyamidino)phenoxy)propane; 1,4-bis-(4′-(N-hydroxyamidino)phenoxy)butane; 1,5-bis-(4′-(N-hydroxyamidino)phenoxy)pentane; 1,4-bis-(4′-(N-hydroxyamidino)phenoxy)butane; 1,3-bis-(4′-(4-hydroxyamidino)phenoxy)propane; 1,3-bis-(2′-methoxy-4′-(N-hydroxyamidino) phenoxy)propane; 2,5-bis-[4-amidinophenyl]furan; 2,5-bis-[4-amidinophenyl]furan bis-amidoxime; 2,5-bis-[4-amidinophenyl]furan bis-O-methylamidoxime; 2,5-bis-[4-amidinophenyl]furan bis-O-ethylamidoxime; 2,8-diamidinodibenzothiophene; 2,8-bis-(N-isopropylamidino) carbazole; 2,8-bis-(N-hydroxyamidino)carbazole; 2,8-bis-(2-imidazolinyl)dibenzothiophene; 2,8-bis-(2-imidazolinyl)-5,5-dioxodibenzothiophene; 3,7-diamidinodibenzothiophene; 3,7-bis-(N-isopropylamidino)dibenzothiophene; 3,7-bis-(N-hydroxyamidino)dibenzothiophene; 3,7-diaminodibenzothiophene; 3,7-dibromodibenzothiophene; 3,7-dicyanodibenzothiophene; 2,8-diamidinodibenzofuran; 2,8-di(2-imidazolinyl)dibenzofuran; 2,8-di(N-isopropylamidino)dibenzofuran; 2,8-di(N-hydroxylamidino)dibenzofuran; 3,7-di(2-imidazolinyl)dibenzofuran; 3,7-di(isopropylamidino)dibenzofuran; 3,7-di(A-hydroxylamidino)dibenzofuran; 2,8-dicyanodibenzofuran; 4,4′-dibromo-2,2′-dinitrobiphenyl; 2-methoxy-2′-nitro-4,4′-dibromobiphenyl; 2-methoxy-2′-amino-4,4′-dibromobiphenyl; 3,7-dibromo-dibenzofuran; 3,7-dicyano-dibenzofuran; 2,5-bis-(5-amidino-2-benzimidazolyl)pyrrole; 2,5-bis-[5-(2-imidazolinyl)-2-benzimidazolyl]pyrrole; 2,6-bis-[5-(2-imidazolinyl)-2-benzimidazolyl]pyridine; 1-methyl-2,5-bis-(5-amidino-2-benzimidazolyl)pyrrole; 1-methyl-2,5-bis-[5-(2-imidazolyl)-2-benzimidazolyl]pyrrole; 1-methyl-2,5-bis-[5-(1,4,5,6-tetrahydro-2-pyrimidinyl)-2-benzimidazolyl]pyrrole; 2,6-bis-(5-amidino-2-benzimidazoyl)pyridine; 2,6-bis-[5-(1,4,5,6-tetrahydro-2-pyrimidinyl)-2-benzimidazolyl]pyridine; 2,5-bis-(5-amidino-2-benzimidazolyl)furan; 2,5-bis-[5-(2-imidazolinyl)-2-benzimidazolyl]furan; 2,5-bis-(5-N-isopropylamidino-2-benzimidazolyl)furan; 2,5-bis-(4-guanylphenyl)furan; 2,5-bis(4-guanylphenyl)-3,4-dimethylfuran; 2,5-di-p[2(3,4,5,6-tetrahydropyrimidyl)phenyl]furan; 2,5-bis-[4-(2-imidazolinyl)phenyl]furan; 2,5-[bis-{4-(2-tetrahydropyrimidinyl)}phenyl]-p(tolyloxy)furan; 2,5-[bis{4-(2-imidazolinyl)}phenyl]3-p(tolyloxy)furan; 2,5-bis-{4-[5-(N-2-aminoethylamido)benzimidazol-2-yl]phenyl}furan; 2,5-bis[4-(3a,4,5,6,7,7a-hexahydro-1H-benzimidazol-2-yl)phenyl]furan; 2,5-bis-[4-(4,5,6,7-tetrahydro-1H-1,3-diazepin-2-yl)phenyl]furan; 2,5-bis-(4-N,N-dimethylcarboxhydrazidephenyl)furan; 2,5-bis-{4-[2-(N-2-hydroxyethyl)imidazolinyl]-phenyl}furan; 2,5-bis[4-(N-isopropylamidino)phenyl]furan; 2,5-bis-{4-[3-(dimethylaminopropyl)amidino]phenyl}furan; 2,5-bis-{4-[N-(3-aminopropyl)amidino]phenyl}furan; 2,5-bis-[2-(imidzaolinyl)phenyl]-3,4-bis(methoxymethyl)furan; 2,5-bis-[4-N-(dimethylaminoethyl)guanyl]phenylfuran; 2,5-bis-{4-[(N-2-hydroxyethyl)guanyl]phenyl}furan; 2,5-bis-[4-N-(cyclopropylguanyl)phenyl]furan; 2,5-bis-[4-(N,N-diethylaminopropyl)guanyl]phenylfuran; 2,5-bis-{4-[2-(N-ethylimidazolinyl)]phenyl}furan; 2,5-bis-{4-[N-(3-pentylguanyl)]}phenylfuran; 2,5-bis-[4-(2-imidazolinyl)phenyl]-3-methoxyfuran; 2,5-bis-[4-(N-isopropylamidino)phenyl]-3-methylfuran; bis-[5-amidino-2-benzimidazolyl]methane; bis-[5-(2-imidazolyl)-2-benzimidazolyl]methane; 1,2-bis-[5-amidino-2-benzimidazolyl]ethane; 1,2-bis-[5-(2-imidazolyl)-2-benzimidazolyl]ethane; 1,3-bis-[5-amidino-2-benzimidazolyl]propane; 1,3bis-[5-(2-imidazolyl)-2-benzimidazolyl]propane; 1,4-bis-[5-amidino-2-benzimidazolyl]propane; 1,4-bis-[5-(2-imidazolyl)-2-benzimidazolyl]butane; 1,8-bis-[5-amidino-2-benzimidazolyl]octane; trans-1,2-bis-[5-amidino-2-benzimidazolyl]ethene; 1,4-bis-[5-(2-imidazolyl)-2-benzimidazolyl]1-butene; 1,4-bis-[5-(2-imidazolyl)-2-benzimidazolyl]2-butene; 1,4-bis-[5-(2-imidazolyl)-2-benzimidazolyl]1-methylbutane; 1,4-bis-[5-(2-imidazolyl)-2-benzimidazolyl]2-ethylbutane; 1,4-bis-[5-(2-imidazolyl)-2-benzimidazolyl 1-methyl-1-butene; 1,4-bis-[5-(2-imidazolyl)-2-benzimidazolyl]2,3-diethyl-2-butene; 1,4-bis-[5-(2-imidazolyl)-2-benzimidazolyl]1,3-butadiene; 1,4-bis-[5-(2-imidazolyl)-2-benzimidazolyl]2-methyl-1,3-butadiene; bis-[5-(2-pyrimidyl)-2-benzimidazolyl]methane; 1,2-bis-[5-(2-pyrimidyl)-2-benzimidazolyl]ethane; 1,3-bis-[5-amidino-2-benzimidazolyl]propane; 1,3-bis-[5-(2-pyrimidyl)-2-benzimidazolyl]propane; 1,4-bis-[5-(2-pyrimidyl)-2-benzimidazolyl]butane; 1,4-bis-[5-(2-pyrimidyl)-2-benzimidazol]1-butene; 1,4-bis-[5-(2-pyrimidyl)-2-benzimidazolyl]2-butene; 1,4-bis-[5-2-pyrimidyl)-2-benzimidazolyl]1-methylbutane; 1,4-bis-[5-(2-pyrimidyl)-2-benzimidazolyl]2-ethylbutane; 1,4-bis-[5-(2-pyrimidyl)-2-benzimidazolyl]1-methyl-1-butene; 1,4-bis-[5-(2-pyrimidyl)-2-benzimidazolyl]2,3-diethyl-2-butene; 1,4-bis-[5-(2-pyrimidyl)-2-benzimidazolyl]1,3-butadiene; and 1,4-bis-[5-(2-pyrimidyl)-2-benzimidazolyl]2-methyl-1,3-butadiene; 2,4-bis-(4-guanylphenyl)-pyrimidine; 2,4-bis-(4-imidazolin-2-yl)-pyrimidine; 2,4-bis-[(tetrahydropyrimidinyl-2-yl)phenyl]pyrimidine; 2-(4-[N-i-propylguanyl]phenyl)-4-(2-methoxy-4-[N-i-propylguanyl]phenyl)pyrimidine; 4-(N-cyclopentylamidino)-1,2-phenylene diamine; 2,5-bis-[2-(5-amidino)benzimidazoyl]furan; 2,5-bis-[2-{5-(2-imidazolino)}benzimidazoyl]furan; 2,5-bis-[2-(5-N-isopropylamidino) benzimidazoyl]furan; 2,5-bis-[2-(5-N-cyclopentylamidino)benzimidazoyl]furan; 2,5-bis[2-(5-amidino)benzimidazoyl]pyrrole; 2,5-bis-[2-{5-(2-imidazolino)}benzimidazoyl]pyrrole; 2,5-bis[2-(5-N-isopropylamidino)benzimidazoyl]pyrrole; 2,5-bis-[2-(5-N-cyclopentylamidino)benzimidazoyl]pyrrole; 1-methyl-2,5-bis-[2-(5-amidino)benzimidazoyl]pyrrole; 2,5-bis-[2-{5-(2-imidazolino)}benzimidazoyl]-1-methylpyrrole; 2,5-bis[2-(5-N-cyclopentylamidino)benzimidazoyl]1-methylpyrrole; 2,5-bis-[2-(5-N-isopropylamidino)benzimidazoyl]thiophene; 2,6-bis-[2-{5-(2-imidazolino)}benzimidazoyl]pyridine; 2,6-bis-[2-(5-amidino)benzimidazoyl]pyridine; 4,4′-bis-[2-(5-N-isopropylamidino)benzimidazoyl]1,2-diphenylethane; 4,4′-bis-[2-(5-N-cyclopentylamidino)benzimidazoyl]-2,5-diphenylfuran; 2,5-bis-[2-(5-amidino) benzimidazoyl]benzo[b]furan; 2,5-bis-[2-(5-N-cyclopentylamidino)benzimidazoyl]benzo[b]furan; 2,7-bis-[2-(5-N-isopropylamidino)benzimidazoyl]fluorine; 2,5-bis-[4-(3-(N-morpholinopropyl)carbamoyl)phenyl]furan; 2,5-bis-[4-(2-N,N-dimethylaminoethylcarbamoyl)phenyl]furan; 2,5-bis-[4-(3-N,N-dimethylaminopropylcarbamoyl)phenyl]furan; 2,5-bis-[4-(3-N-methyl-3-N-phenylaminopropylcarbamoyl)phenyl]furan; 2,5-bis-[4-(3-N,N⁸,N¹¹-trimethylaminopropylcarbamoyl)phenyl]furan; 2,5-bis-[3-amidinophenyl]furan; 2,5-bis-[3-(N-isopropylamidino)amidinophenyl]furan; 2,5-bis[3[(N-(2-dimethylaminoethyl)amidino]phenylfuran; 2,5-bis-[4-(N-2,2,2-trichloroethoxycarbonyl)amidinophenyl]furan; 2,5-bis-[4-(N-thioethylcarbonyl) amidinophenyl]furan; 2,5-bis-[4-(N-benzyloxycarbonyl)amidinophenyl]furan; 2,5-bis[4-(N-phenoxycarbonyl)amidinophenyl]furan; 2,5-bis-[4-(N-(4-fluoro)-phenoxycarbonyl)amidinophenyl]furan; 2,5-bis-[4-(N-(4-methoxy) phenoxycarbonyl)amidinophenyl]furan; 2,5-bis-[4(1-acetoxyethoxycarbonyl) amidinophenyl]furan; and 2,5-bis-[4-(N-(3-fluoro)phenoxycarbonyl)amidinophenyl]furan (or a salt of any of the above).

In certain embodiments, the above drug combinations may further comprise an antiproliferative agent.

In certain embodiments, the drug combinations may comprise a first compound as listed above and an antiproliferative agent.

In certain other embodiments, the drug combinations may comprise a second compound as listed above and an antiproliferative agent.

In certain embodiments, the drug combinations comprise a first compound selected from alberdazole, mebendazole, oxibendazole, or a salt thereof and a second compound is pentamidine or a salt thereof.

In certain embodiments, the drug combinations of the present invention may comprise albendazole and pentamidine isethionate. In certain other embodiments, the drug combinations of the present invention may comprise albendazole sulfoxide and pentamidine isethionate, mebendazole and pentamidine isethionate, or oxibendazole and pentamidine isethionate.

In certain embodiments, the drug combinations of the present invention may comprise albendazole and 2,5-bis-[4-amidinophenyl]furan bis-O-methylamidoxime.

In certain embodiments, the drug combinations of the present invention may comprise albendazole and 2,5-bis-[4-amidinophenyl]furan.

Combinations Comprising Dibucaine or Amide Local Anaesthetics Related to Bupivacaine and Vinca Alkaloids

In certain embodiments, the drug combinations according to the present invention may comprise (1) a dibucaine or amide local anaestheic related to bupivacaine (or structural or functional analogues, salts, or metabolites) and (2) a vinca alkaloid (or structural or functional analogues, salts, or metabolites). In certain embodiments, the drug combination may further comprise one or more antiproliferative agents (e.g., those listed in Table 1)

Dibucaine and Amide Local Anaesthetics Related to Bupivacaine

Compounds of Formula (XXIX)

Compounds of formula (XXIX) have the formula:

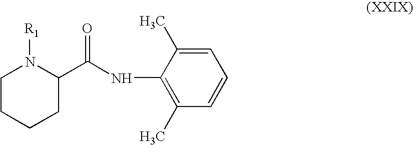

wherein R₁ is H, OH, a halide, or any branched or unbranched, substituted or unsubstituted C₁₋₁₀ alkyl, C₁₋₁₀ alkoxyalkyl, C₁₋₁₀ hydroxyalkyl, C₁₋₁₀ aminoalkyl, C₁₋₁₀ alkylaminoalkyl, C₄₋₁₀ cycloalkyl, C₅₋₈ aryl, or C₆₋₂₀ alkylaryl; most preferably R₁ is CH₃—, CH₃CH₂CH₂—, or CH₃CH₂CH₂CH₂—.

Exemplary compounds of this formula are bupivacaine (1-butyl-2′,6′-pipecoloxylidide), levobupivacaine (also called chirocaine; (S)-1-butyl-2′,6′-pipecoloxylidide), mepivacaine ((+/−)-1-methyl-2′,6′-pipecoloxylidide), and ropivacaine ((−)-1-propyl-2′,6′-pipecoloxylidide). These compounds are tertiary amide local anaesthetics. Local anaesthetics block the initiation and propagation of action potentials by preventing the voltage-dependent increase in Na+conductance. They can be used for surgical anesthesia and postoperative pain management. For surgical anesthesia, bupivacaine has been approved for epidural use, peripheral neural blockade, and local infiltration as well as for pain management. Typically, a 0.75% solution of bupivacaine is administered for ophthalmic surgery. A 0.5% bupivacaine solution may be administered for Cesarean section or peripheral nerve block. A 0.25% solution of bupivacaine may be administered in infiltration anaesthesia or to women in early labor requesting epidural analgesia. A composition of 0.125% bupivacaine may be used for postoperative pain management. Levobupivacaine and ropivacaine have similar administration, while mepivacaine is ineffective as a topical anaesthetic.

Compounds of Formula (XXX)

Compounds of formula (XXX) have the formula:

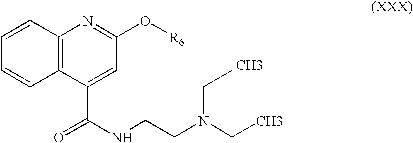

wherein R₆ is —(CH)₂)₂OCH₃, —(CH)₂)₂OCH₂CH₃, or —((CH)₂)₃CH₃. An exemplary member of this class is dibucaine(2-butoxy-N-(2-(diethylamino)ethyl)cinchoninamide), which has the formula (XXXI):

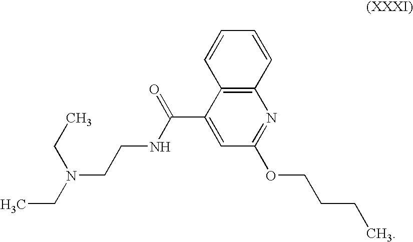

Dibucaine(2-butoxy-N-(2-(diethylamino)ethyl)cinchoninamide) is used as a topical analgesic, anaesthetic and antipruritic for the temporary relief of pain and itching due to minor burns, sunburn, minor cuts, abrasions, insect bites and minor skin irritations. It is typically formulated as a 0.5% to 1% solution.

Vinca Alkaloids—Compounds of Formula (XXXII)

“Vinca alkaloid” refers to a compound of formula (XXXII), which encompasses plant-derived antiproliferative compound such as vinblastine, vinleurosine, vinrosidine or vincristine (each found in the Madagascar periwinkle, Catharanthus roseus) as well as the semi-synthetic derivatives such as vindesine and vinorelbine. They are antineoplastic agents that act by binding tubulin and inhibiting its polymerization into microtubules.

Examples of vinca alkaloids are vinblastine, vinorelbine, vindesine, and vincristine.

Compounds of formula (XXXII) have the formula:

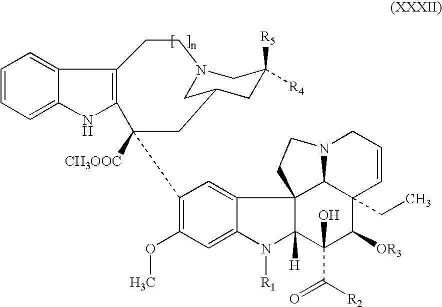

wherein R₁ is CHO, CH₃, or H, R₂ is OCH₃ or NH₂, R₃ is OCOCH₃ or OH, R₄ is H, CH₃, CH₂CH₃, or CF₂CH₃, R₅ is H, OH, or CH₂CH₃, and n=0 or 1.

Antiproliferative Agents

Antiproliferative agents are described above. They include, but are not limited to microtubule inhibitors, topoisomerase inhibitors, platins, alkylating agents, and anti-metabolites. Exemplary antiproliferative agents useful in the present application include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine. Additional antiproliferative agents may be found in Table 1.

Exemplary Drug Combinations

In certain embodiments, the drug combinations of the present invention may comprise (1) a first compound selected from bupivacaine, levobupivacaine, ropivacaine, and mepivacaine, and (2) a second compound selected from vinblastine, vincristine, vindestine, or vinorelbine.

In certain other embodiments, the drug combinations of the present invention may comprise dibucaine and a second compound selected from vinblastine, vincristine, vindestine, or vinorelbine.

In certain embodiments, the drug combinations of the present invention may comprise bupivacaine and vinblastine, levobupivacaine and vinblastine, dibucaine and vinblastine, mepivacaine and vinblastine, ropivacaine and vinblastine.

In certain embodiment, the drug combinations of the present invention may comprise levobupivicaine and vinorelbine, or dibucaine and vinorelbine.

Combinations Comprising Pentamidine and Antiproliferative Agents

In certain embodiments, the drug combinations according to the present invention may comprise pentamidine (or its structural or functional analogs, salts, or metabolites) and an antiproliferative agent.

Pentamidine, Analogs, Salts, and Metabolites

Pentamidine, its analogs, pharmaceutically active or acceptable salts and metabolites are described as above in the section related to combinations comprising chlorpromazine and pentamidine.

In certain embodiments, pentamidine analogs have formula (XXXIII) 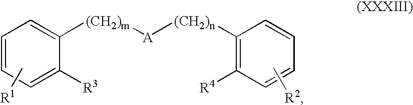
or a pharmaceutically acceptable salt thereof,

wherein A is

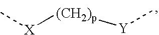

each of X and Y is, independently, O or NH,

p is an integer between 2 and 6, inclusive,

each of m and n is, independently, an integer between 0 and 2, inclusive, wherein the sum of m and n is greater than 0,

each of R¹ and R² is, independently, selected from the group represented by

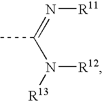

wherein R¹² is H, C₁-C₆ alkyl, C₁-C₈ cycloalkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, or, R¹³ is H, C₁-C₆ alkyl, C₁-C₈ cycloalkyl, C₆-C₁₈ aryloxy C₁-C₆ alkyl, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl-C₁C₆ alkoxy), carbo(C₆-C₁₈ aryloxy), or C₆-C₁₈ aryl, and R¹¹ is H, OH, or oxy(C₁-C₆ alkyl), or R¹¹ and R₁₂ together represent

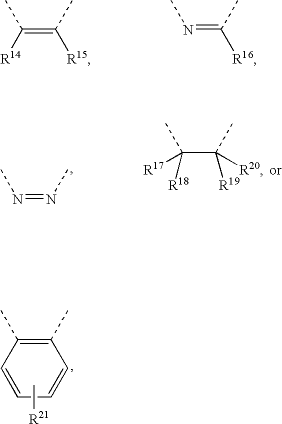

wherein each of R¹⁴, R¹⁵, and R¹⁶ is, independently, H, C₁C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₉ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,

each of R³ and R⁴ is, independently, H, Cl, Br, OH, OCH₃, OCF₃, NO₂, and NH₂, or R³ and R⁴ together form a single bond.

In certain embodiments, A is

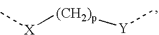

each of X and Y is, independently, O or NH,

p is an integer between 2 and 6, inclusive,

each of m and n is 0, and

each of R¹ and R² is, independently, selected from the group represented by

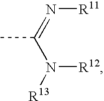

wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R₁₂ together represent

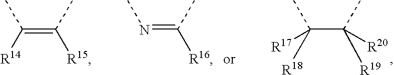

wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl.

In certain embodiments, A is

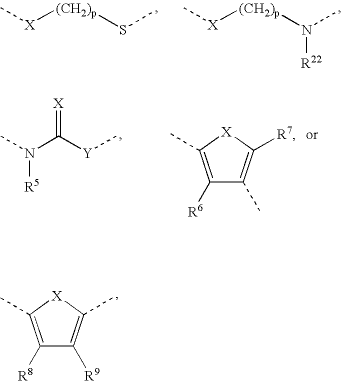

each of X and Y is, independently, O, NR¹⁰, or S, each of R⁵ and R¹⁰ is, independently, H or C₁-C₆ alkyl, each of R⁶, R⁷, R⁸, and R₉ is, independently, H, C₁-C₆ alkyl, halogen, C₁-C₆ alkyloxy, C₆-C₁₈ aryloxy, or C₆-C₁₈ aryl C₁-C₆ alkyloxy,

R²² is C₁-C₆ alkyl,

p is an integer between 2 and 6, inclusive,

each of m and n is, independently, an integer between 0 and 2, inclusive,

each of R¹ and R² is, independently, selected from the group represented by

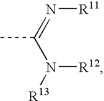

wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₆-C₁₈ aryloxy C₁-C₆ alkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent

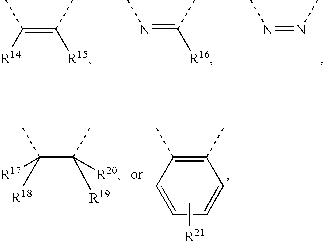

wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl, and

each of R³ and R⁴ is, independently, H, Cl, Br, OH, OCH₃, OCF₃, NO₂, and NH₂, or R³ and R⁴ together form a single bond.

Antiproliferative Agents

Antiproliferative agents useful in combination with pentamidine include both Group A antiproliferative agents and Group B antiproliferative agents.

“Group A antiproliferative agent” refers to any antiproliferative agent that is not a Group B antiproliferative agent.

Examples of Group A agents are those listed in Table 1. Group A antiproliferative agents of the invention also include those alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors that are not Group B antiproliferative agents, as defined herein (see Table 2).

In certain embodiments, the Group A antiproliferative agent is vinblastine, carboplatin, etoposide, or gemcitabine. “Group B antiproliferative agent” refers to any antiproliferative agent selected from the group of compounds in Table 6.

TABLE 6
(Group B)

melphalan
carmustine
cisplatin
5-fluorouracil
mitomycin C
adriamycin (doxorubicin)
bleomycin
Paclitaxel (Taxol ®)

Exemplary Drug Combinations

In one embodiment, the combinations of the present invention comprises (1) a compound of formula (XXXIII) selected from pentamidine, propamidine, butamidine, heptamidine, nonamidine, dibrompropamidine, 1,3-bis(4-amidino-2-methoxyphenoxy)propane, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-0-methylamidoxime, 2,5-bis[4-amidinophenyl]furan-bis-0-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,5-bis[4-(N-isopropylamidino)phenyl]furan, 2,5-bis {4-[3-(dimethylaminopropyl)amidino]phenyl}furan, 2,5-bis{4-[N-(3-aminopropyl)amidino]phenyl}furan, 2,5-bis[4-(2-imidazolinyl)phenyl]-3-methoxyfuran, 2,5-bis[4-(N-isopropylamidino)phenyl]-3-methylfuran, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis [4(1-acetoxyethoxycarbonyl)amidinophenyl]furan, 2,5-bis[4-(N-(3-fluoro)phenoxycarbonyl)amidinophenyl]furan, and pharmaceutically active or acceptable salts of the above listed agents, and (2) an antiproliferative agent selected from vinblastine, carboplatin, adriamycin(doxorubicin), etoposide, and gemcitabine.

In certain embodiments, the drug combinations comprise (1) a compound selected from pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, and pharmaceutically active or acceptable salts thereof, and (2) an antiproliferative agent selected from vinblastine, carboplatin, adriamycin(doxorubicin), etoposide, and gemcitabine.

In certain embodiments, the drug combinations comprise (1) an endo-exonuclease inhibitor and (2) one or more Group A antiproliferative agents (e.g., vinblastine, carboplatin, etoposide, and gemcitabine).

In certain embodiments, the drug combinations comprise (1) a phosphatase of regenerating liver (PRL) inhibitor or a PTB1B inhibitor and (2) one or more Group A antiproliferative agents (e.g., vinblastine, carboplatin, etoposide, or gemcitabine).

In certain embodiments, the drug combinations comprise pentamidine and vinblastine, pentamidine and carboplatin, pentamidine and doxorubicin, pentamidine and etoposide, pentamidine and gemcitabine, or pentamidine and 5-fluorouracil.

Combinations Comprising Triazoles and Antiarrhythmic Agents

In certain embodiments, the drug combinations according to the present invention may comprise triazoles (or their structural or functional analogs, pharmaceutically active or acceptable salts, or metabolites) and antiarrhythmic agents (or their structural or functional analogs, pharmaceutically active or acceptable salts, or metabolites). In certain embodiments, the drug combinations may further comprise one or more antiproliferative agents.

Antiarrhythmic Agents

“Antiarrhythmic agent” refers to a drug that reduces cardiac arrhythmia. Examples of antiarrhythmic agents are drugs that block voltage-sensitive sodium channels, beta-adrenoceptor antagonists, drugs that prolong the cardiac action potential, and Ca²⁺channel antagonists.

Generally, there is little structure-activity relationship between antiarrhythmic agents with regard to their antiarrhythmic effects. By the Vaughan Williams' classification, antiarrhythmic agents are generally divided into four classes.

Class I drugs block voltage-sensitive sodium channels. Class I drugs are further divided into Classes IA, IB and IC. Class IA drugs lengthen the duration of the myocardial action potential while decreasing the maximal rate of depolarization. Class IA drugs include hydroxyl quinidine, quinidine, disopyramide, and procainamide. Class IB antiarrhythmic agents decrease the maximal rate of depolarization as well as decreasing the duration of the myocardial action potential. Examples of Class IB agents are lidocaine, tocainide, mexiletine, and phenytoin. Class IC antiarrhythmic agents decrease the maximal rate of depolarization while having no effect on the duration of the myocardial action potential. Examples include flecainide and encainide.

Class II drugs are beta-adrenoceptor antagonists, examples of which are propranolol, acebutolol, esmolol, and sotalol.

Class III drugs prolong the cardiac action potential, thereby increasing the refractory period suppressing the ectopic and re-entrant activity, such as amiodarone, sotalol, and bretylium tosylate.

Class IV drugs are Ca2+ channel antagonists, which block the slow inward current that is carried by calcium ions during the myocardial action potential. Examples of Class IV drugs are nifedipine, amlodipine, felodipine, flunarizine, isradipine, nicardipine, diltiazem, verapamil, and bepridil.

Other antiarrhythmic agents that do not fall within one of the above categories but are considered antiarrhythmic agents include digoxin and adenosine.

Amiodarone

Amiodarone (2-Butyl-3-benzofuranyl)(4-(2-(diethylamino)ethoxy)-3,5-diidophenyl)methanone; Cordarone™) has the following structure:

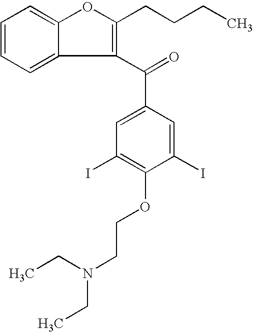

Related compounds to amiodarone include di-N-desethylamiodarone, desethylamiodarone, desoxoamiodarone, etabenzarone, and 2-butylbenzofuran-3-yl, 4 hydroxy-3,5-diiodophenyl ketone.

Bepridil

Bepridil (beta-((2-methylpropoxy)methyl)-N-phenyl-N-(phenylmethyl)-1-pyrrolidineethanamine) has the following structure: 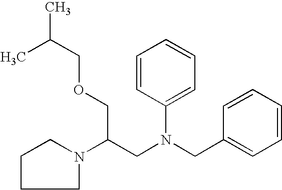
Nicardipine

Nicardipine (2-(benzyl-methyl amino)ethyl methyl 1,4-dihydro-2,6-dimethyl-4-(m-nitrophenyl)-3,5-pyridinedicarboxylate monohydrochloride) is a class IV antiarrhythmic having the following structure:

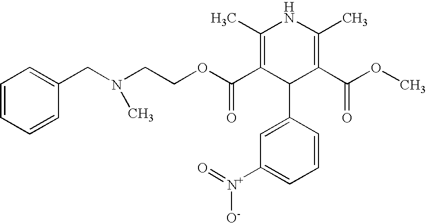

Additional antiarrhythmic agents include amlodipine, nifedipine, diltiazem, felodipine, flunarizine, isradipine, nimodipine, and verapamil. Triazoles

“Triazole” refers to a compound having a five-membered ring of two carbon atoms and three nitrogen atoms. Triazoles useful in the present invention may have formula (XXXIV):

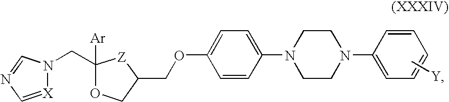

or a pharmaceutically active or acceptable salt thereof, wherein X is CH₂ or N; Z is CH₂ or O; Ar is selected from the group consisting of phenyl, thienyl, halothienyl, and substituted phenyl having from 1 to 3 substituents, each independently selected from the group consisting of halo, C₁-C₆ linear or branched alkyl, linear or branched C₁-C₆ alkoxy, and trifluoromethyl; and Y is a group having the formula:

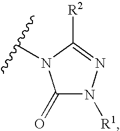

wherein R¹ is selected from the group consisting of C₁-C₆ linear or branched alkyl having 0 or 1 hydroxyl substituents and C₁-C₆ linear or branched alkaryl, and R² is selected from the group consisting of H, linear or branched C₁-C₆ alkyl, and C₁-C₆ alkaryl, wherein said aryl group is a phenyl ring having from 0 to 3 substituents, each independently selected from the group consisting of halo, C₁-C₆ linear or branched alkyl, linear or branched C₁-C₆ alkoxy, and trifluoromethyl. Exemplary triazoles of formula (XXXIV) include itraconazole, hydroxyitraconazole, posaconazole, and saperconazole.

Antiproliferative Agents

Antiproliferative agents are described above. Exemplary antiproliferative agents include cisplatin, daunorubicin, doxorubicin, etoposide, methotrexate, mercaptopurine, 5-fluorouracil, hydroxyurea, vinblastine, vincristine, paclitaxel, bicalutamide, bleomycin, carboplatin, carmustine, cyclophosphamide, docetaxel, epirubicin, gemcitabine hcl, goserelin acetate, imatinib, interferon alpha, irinotecan, lomustine, leuprolide acetate, mitomycin, rituximab, tamoxifen, trastuzumab, or any combination thereof.

Exemplary Drug Combinations

In certain embodiments, the drug combinations according to the present invention comprise (1) an antiarrhythmic agent selected from amiodarone, di-N-desethylamiodarone, desethylamiodarone, bepridil, and nicardipine, and (2) a triazole selected from itraconazole, hydroxyitraconazole, posaconazole, and saperconazole.

In certain embodiments, the drug combinations comprise itraconazole and amiodarone, bepridil and itraconazole, or itraconazole and nicardipine.

Combinations Comprising Azoles and HMG-CoA Reductase Inhibitors

In certain embodiments, the drug combinations according to the present invention may comprise azoles (or their structural or functional analogs, pharmaceutically active or acceptable salts, or metabolites) and HMG-CoA reductase inhibitors (or their structural or functional analogs, pharmaceutically active or acceptable salts, or metabolites). In certain embodiments, the drug combinations may further comprise one or more antiproliferative agents.

HMG-CoA Reductase Inhibitors

“HMG-CoA reductase inhibitor” refers to a compound that inhibits the enzymatic activity of 3-hydroxy-3-methylglutaryl-coenzyme A (HMG-CoA) reductase by at least about 10%. HMG-CoA reductase inhibitors include but are not limited to simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin, as well as pharmaceutically active or acceptable salts thereof (e.g., simvastatin sodium, lovastatin sodium, fluvastatin sodium, etc.).

Additional HMG-CoA reductase inhibitors and analogs thereof useful in the methods and compositions of the present invention are described in U.S. Pat. Nos. 3,983,140; 4,231,938; 4,282,155; 4,293,496; 4,294,926; 4,319,039; 4,343,814; 4,346,227; 4,351,844; 4,361,515; 4,376,863; 4,444,784; 4,448,784; 4,448,979; 4,450,171; 4,503,072; 4,517,373; 4,661,483; 4,668,699; 4,681,893; 4,719,229; 4,738,982; 4,739,073; 4,766,145; 4,782,084; 4,804,770; 4,841,074; 4,847,306; 4,857,546; 4,857,547; 4,940,727; 4,946,864; 5,001,148; 5,006,530; 5,075,311; 5,112,857; 5,116,870; 5,120,848; 5,166,364; 5,173,487; 5,177,080; 5,273,995; 5,276,021; 5,369,123; 5,385,932; 5,502,199; 5,763,414; 5,877,208; and 6,541,511; and U.S. Patent Application Publication Nos. 2002/0013334 A1; 2002/0028826 A1; 2002/0061901 A1; and 2002/0094977 A1.

Azoles

“Azole” refers to any member of the class of antifungal compounds having a five-membered ring of three carbon atoms and two nitrogen atoms (e.g., imidazoles) or two carbon atoms and three nitrogen atoms (e.g., triazoles), which are capable of inhibiting fungal growth. A compound is considered “antifungal” if it inhibits growth of a species of fungus in vitro by at least 25%.

Azoles that can be employed in the methods and compositions of the invention include fluconazole, itraconazole, hydroxyitraconazole, posaconazole, saperconazole, ketoconazole, clotrimazole, terconazole, econazole, tioconazole, oxiconazole, butoconazole, and miconazole.

Additional azoles and analogs thereof useful in the methods and compositions of the present invention are described in U.S. Pat. Nos. 3,575,999; 3,705,172; 3,717,655; 3,936,470; 4,062,966; 4,078,071; 4,107,314; 4,124,767; 4,144,346; 4,223,036; 4,229,581; 4,232,034; 4,244,964; 4,248,881; 4,267,179; 4,272,545; 4,307,105; 4,335,125; 4,360,526; 4,368,200; 4,402,968; 4,404,216; 4,416,682; 4,458,079; 4,466,974; 4,483,865; 4,490,530; 4,490,540; 4,503,055; 4,510,148; 4,554,286; 4,619,931; 4,625,036; 4,628,104; 4,632,933; 4,661,602; 4,684,392; 4,735,942; 4,761,483; 4,771,065; 4,789,587; 4,818,758; 4,833,141; 4,877,878; 4,916,134; 4,921,870; 4,960,782; 4,992,454; and 5,661,151.

Antiproliferative Agents

Antiproliferative agents are described above. Exemplary antiprolierative agents include cisplatin, daunorubicin, doxorubicin, etoposide, methotrexate, mercaptopurine, 5-fluorouracil, hydroxyurea, vinblastine, vincristine, paclitaxel, or any combination thereof.

Exemplary Drug Combinations

In certain embodiments, the drug combinations of the present invention comprise (1) an azole selected from fluconazole, itraconazole, hydroxyitraconazole, posaconazole, saperconazole, ketoconazole, clotrimazole, terconazole, econazole, tioconazole, oxiconazole, butoconazole, miconazole, and pharmaceutically active or acceptable salts thereof, and (2) an HMG-CoA reductase inhibitor selected from simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, pitavastatin, and pharmaceutically active or acceptable salts thereof.

In certain embodiments, the drug combinations of the present invention may comprise simvastatin and itraconazole, atorvastatin and itraconazole, fluvastatin and itraconazole, lovastatin and itraconazole, atorvastatin and clotrimazole, atorvastatin and econazole, atorvastatin and ketoconazole, lovastatin and econazole, atorvastatin and terconazole, cerivastatin and itraconazole; or lovastatin and tioconazole.

Combinations Comprising Phenothiazine Conjugates or Phenothiazines and Antiproliferative Agents

In certain embodiments, the drug combinations of the present invention may comprise or be phenothiazine conjugates (e.g., conjugates comprising phenothiazines and antiproliferative agents). The phenothiazine conjugates generally have three characteristic components: a phenothiazine covalently tethered, via a linker, to a group that is bulky or charged.

In certain embodiments, the drug combination may comprise phenothiazines and antiprolierative agents.

Phenothiazine Conjugates

Phenothiazines

By “phenothiazine” is meant any compound having a phenothiazine ring structure or related ring structure as shown below. Thus, ring systems for which the ring sulfur atom is oxidized, or replaced by O, NH, CH₂, or CH=CH are encompassed by the generic description “phenothiazine.” For all of the ring systems show below, phenothiazines include those ring substitutions and nitrogen substitutions provide for in formulas ((VI)(A)) and (VII).

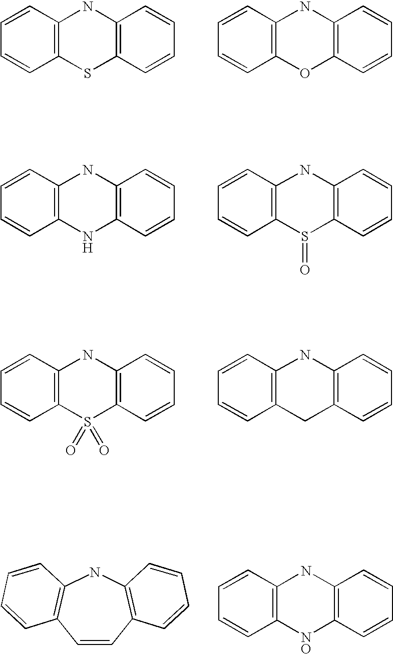

By “parent phenothiazine” is meant the phenothiazine which is modified by conjugation to a bulky group or a charged group. A phenothiazine conjugate includes a phenothiazine covalently attached via a linker to a bulky group of greater than 200 daltons or a charged group of less than 200 daltons.

In certain embodiments, the phenothiazine conjugate is described by formula (VII):

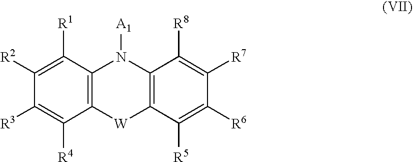

In formula (VII), R² is selected from the group consisting of: CF₃, halogen, OCH₃, COCH₃, CN, OCF₃, COCH₂CH₃, CO(CH₂)₂CH₃, S(O)₂CH₃, S(O)₂N(CH₃)₂, and SCH₂CH₃; A¹ is selected from the group consisting of G¹,

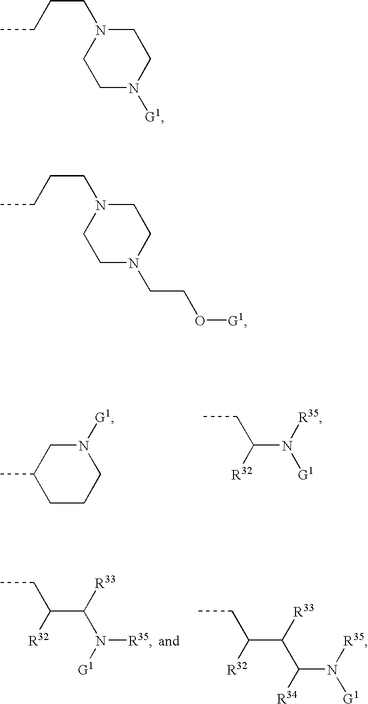

each of R¹, R³, R⁴, R⁵, R⁶, R⁷, and R⁸ is independently H. OH, F, OCF₃, or OCH₃; R³², R³³, R³⁴, and R³⁵, are each, independently, selected from H or C₁₋₆ alkyl; W is selected from the group consisting of: NO,

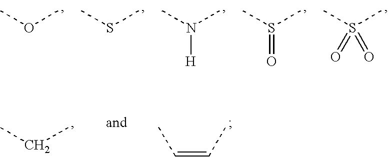

and G¹ is a bond between the phenothiazine and the linker.

Phenothiazines useful in the drug combinations include compounds having a structure as shown in formula (VI)(A): 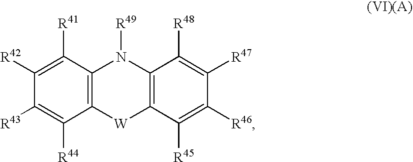
or a pharmaceutically acceptable salt thereof, wherein R⁴² is selected from the group consisting of: CF₃, halogen, OCH₃, COCH₃, CN, OCF₃, COCH₂CH₃, CO(CH₂)₂CH₃, S(O)₂CH₃, S(O)₂N(CH₃)₂, and SCH₂CH₃;
R⁴⁹ is selected from the group consisting of: 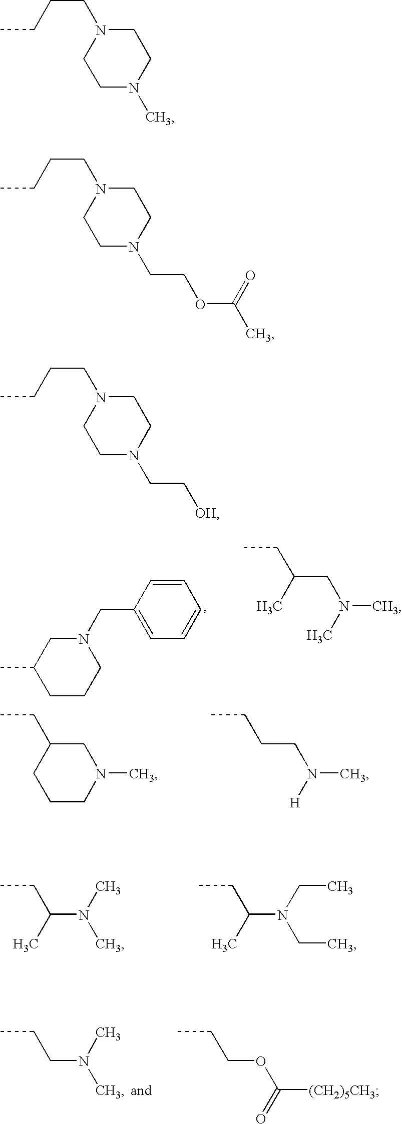
each of R⁴¹, R⁴³, R⁴⁴, R⁴⁵, R⁴⁶, R⁴⁷, and R⁴⁸ is independently H, OH, F, OCF₃, or OCH₃;
and W is selected from the group consisting of: NO,

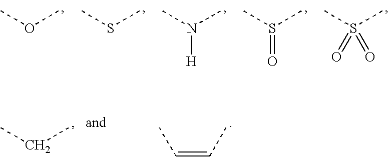

Phenothiazines useful in the present invention include, without limitation, acepromazine, cyamemazine, fluphenazine, mepazine, methotrimeprazine, methoxypromazine, perazine, pericyazine, perimethazine, perphenazine, pipamazine, pipazethate, piperacetazine, pipotiazine, prochlorperazine, promethazine, propionylpromazine, propiomazine, sulforidazine, thiazinaminiumsalt, thiethylperazine, thiopropazate, thioridazine, trifluoperazine, trimeprazine, thioproperazine, trifluomeprazine, triflupromazine, chlorpromazine, chlorproethazine, those compounds in PCT application W002/057244, and those compounds in U.S. Pat. Nos. 2,415,363; 2,519,886; 2,530,451; 2,607,773; 2,645640; 2,766,235; 2,769,002; 2,784,185; 2,785,160; 2,837,518; 2,860,138; 2,877,224; 2,921,069; 2,957,870; 2,989,529; 3,058,979; 3,075,976; 3,194,733; 3,350,268; 3,875,156; 3,879,551; 3,959,268; 3,966,930; 3,998,820; 4,785,095; 4,514,395; 4,985,559; 5,034,019; 5,157,118; 5,178,784; 5,550,143; 5,595,989; 5,654,323; 5,688,788; 5,693,649; 5,712,292; 5,721,254; 5,795,888; 5,597,819; 6,043,239; and 6,569,849, each of which is incorporated herein by reference. Structurally related phenothiazines having similar antiproliferative properties are also intended to be encompassed by this group, which includes any compound of formula ((VI)(A)), described above.

The structures of several of the above-mentioned phenothiazines are provided below. Phenothiazine conjugates of the invention are prepared by modification of an available functional group present in the parent phenothiazine. Alternatively, the substituent at the ring nitrogen can be removed from the parent phenothiazine prior to conjugation with a bulky group or a charged group.

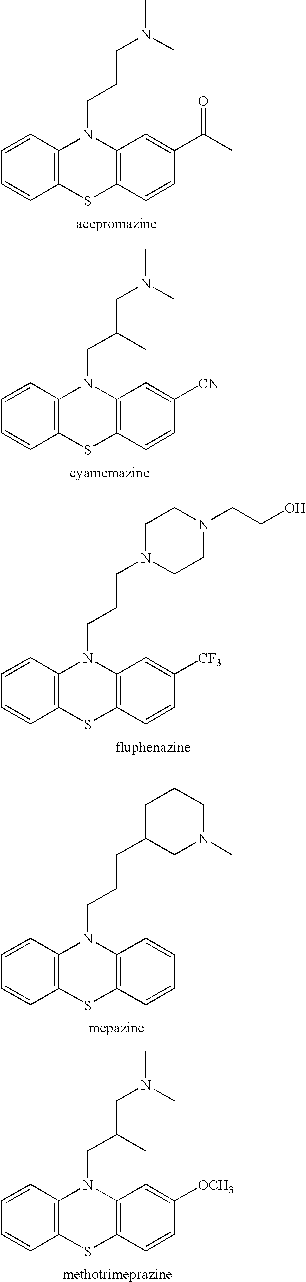

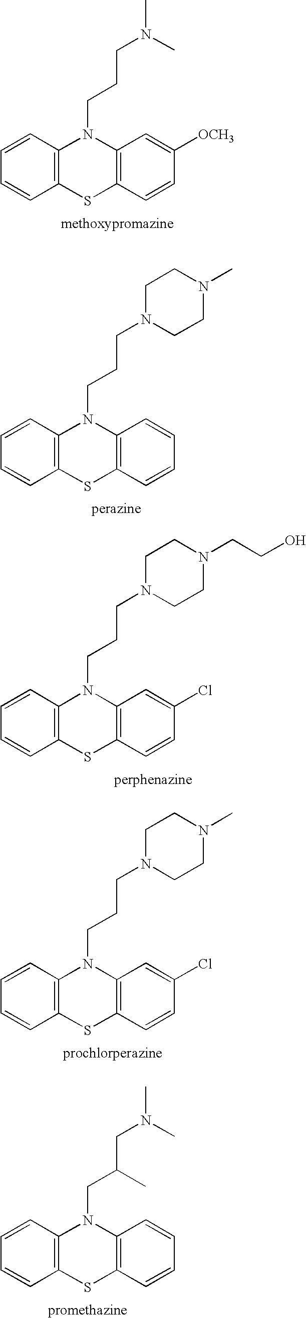

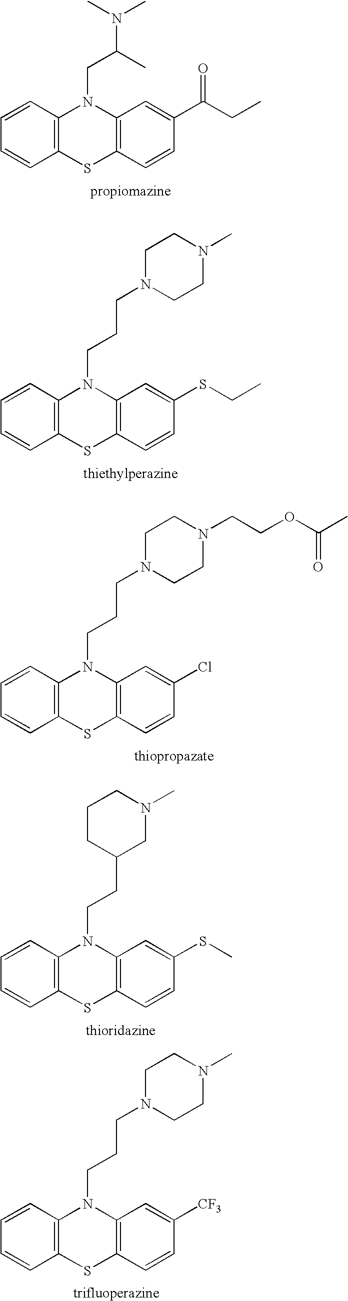

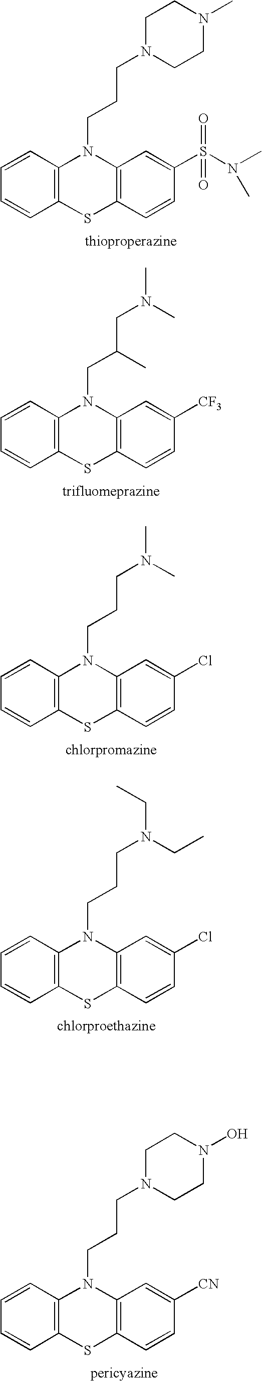

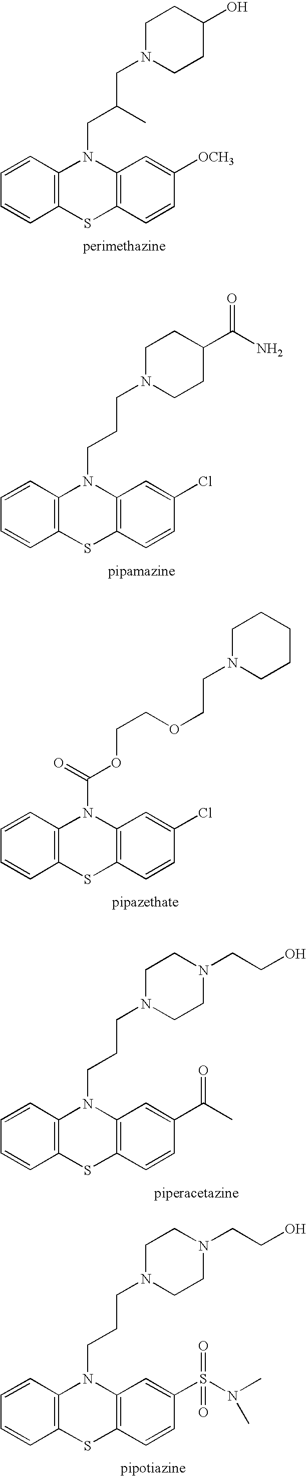

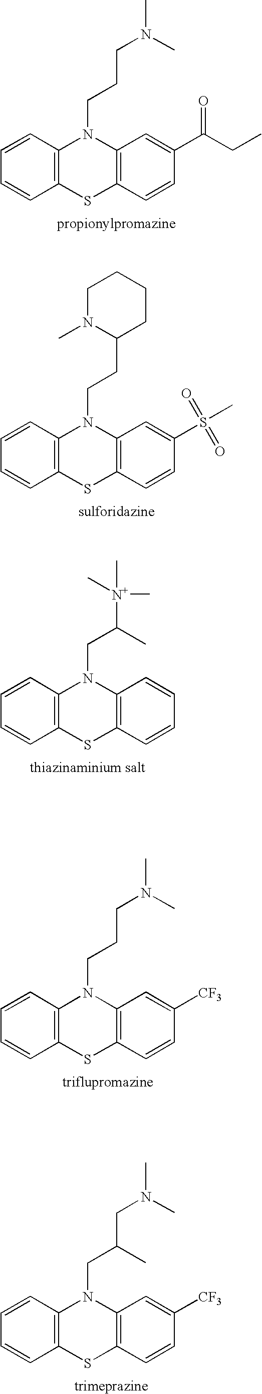

Phenothiazine compounds can be prepared using, for example, the synthetic techniques described in U.S. Pat. Nos. 2,415,363; 2,519,886; 2,530,451; 2,607,773; 2,645640; 2,766,235; 2,769,002; 2,784,185; 2,785,160; 2,837,518; 2,860,138; 2,877,224; 2,921,069; 2,957,870; 2,989,529; 3,058,979; 3,075,976; 3,194,733; 3,350,268; 3,875,156; 3,879,551; 3,959,268; 3,966,930; 3,998,820; 4,785,095; 4,514,395; 4,985,559; 5,034,019; 5,157,118; 5,178,784; 5,550,143; 5,595,989; 5,654,323; 5,688,788; 5,693,649; 5,712,292; 5,721,254; 5,795,888; 5,597,819; 6,043,239; and 6,569,849, each of which is incorporated herein by reference.

Linkers

The linker component of the invention is, at its simplest, a bond between a phenothiazine and a group that is bulky or charged. The linker provides a linear, cyclic, or branched molecular skeleton having pendant groups covalently linking a phenothiazine to a group that is bulky or charged.

Thus, the linking of a phenothiazine to a group that is bulky or charged is achieved by covalent means, involving bond formation with one or more functional groups located on the phenothiazine and the bulky or charged group. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of a phenothiazine and a group that is bulky or charged may be effected using a linker that contains reactive moieties capable of reaction with such functional groups present in the phenothiazine and the bulky or charged group. For example, a hydroxyl group of the phenothiazine may react with a carboxyl group of the linker, or an activated derivative thereof, resulting in the formation of an ester linking the two.

Examples of moieties capable of reaction with sulfhydryl groups include α-haloacetyl compounds of the type XCH₂CO—(where X═Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO—(where X═Cl, Br or I), for example, as described by Wong Biochemistry 24:5337 (1979);

(ii) N-maleimide derivatives, which may react with amino groups either 30 through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff s base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through 0-acylurea formation followed by amide bond formation, may also be employed.

It will be appreciated that functional groups in the phenothiazine and/or the bulky or charged group may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of the phenothiazine with a reactive chemical group of the bulky or charged group without introducing additional linking material may, if desired, be used in accordance with the invention. For example, the ring nitrogen of the phenothiazine can be linked directly via an amide bond to the charged or bulky group.

Most commonly, however, the linker will include two or more reactive moieties, as described above, connected by a spacer element. The presence of such a spacer permits bifunctional linkers to react with specific functional groups within the phenothiazine and the bulky or charged group, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the phenothiazine and the bulky or charged group.

Spacer elements in the linker typically consist of linear or branched chains and may include a C₁₋₁₀ alkyl, a heteroalkyl of 1 to 10 atoms, a C₂₋₁₀ alkene, a C₂₋₁₀ alkyne, C₅₋₁₀ aryl, a cyclic system of 3 to 10 atoms, or —(CH₂CH₂O)ₙCH₂CH₂—, in which

In some instances, the linker is described by formula (XXXV):
G¹-(Z¹)ₒ-(Y¹)_{U}-(Z²)ₛ-(R⁹)-(Z³)ₜ-(Y²)ᵥ-(Z⁴)ₚG² (XXXV)

In formula (II), G¹ is a bond between the phenothiazine and the linker, G² is a bond between the linker and the bulky group or between the linker and the charged group, each of Z¹, Z², Z³, and Z⁴ is, independently, selected from O, S, and NR³⁹; R³⁹ is hydrogen or a C₁₋₁₀ alkyl group; each of Y¹ and Y² is, independently, selected from carbonyl, thiocarbonyl, sulphonyl, phosphoryl or similar acid-forming groups; o, p, s, t, u, and v are each independently 0 or 1; and R⁹ is C₁₋₁₀ alkyl, C₁-lo heteroalkyl, C₂₋₁₀ alkenyl, a C₂₋₁₀ alkynyl, C₅₋₁₀ aryl, a cyclic system of 3 to 10 atoms, or a chemical bond linking G¹-(Z )ₒ-(Y¹)ᵤ-(Z²)ₛ- to -(Z³)ₜ-(Y²)ᵥ-(Z⁴)ₚ-G².

Bulky Groups

In certain embodiments, bulky groups have a molecular weight greater than 200, 300, 400, 500, 600, 700, 800, 900, or 1000 daltons. In certain embodiments, these groups are attached through the ring nitrogen of the phenothiazine.

By “linked through the ring nitrogen” is meant that the charged group, bulky group, or linker is covalently attached to a substituent of ring nitrogen as identified below.

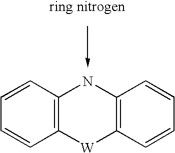

In certain embodiments, the bulky group comprises a naturally occurring polymer, such as a glycoprotein, a polypeptide (alpha-1-acid glycoprotein), or a polysaccharide (e.g., hyaluronic acid). In certain other embodiments, the bulky group comprises a synthetic polymer, such as a polyethylene glycol or N-hxg.

In certain embodiments, a bulky group is a charged bulky group, such as the polyguanidine peptoid (N-hxg)₉, shown below. Each of the nine guanidine side chains is a charged guanidinium cation at physiological pH.

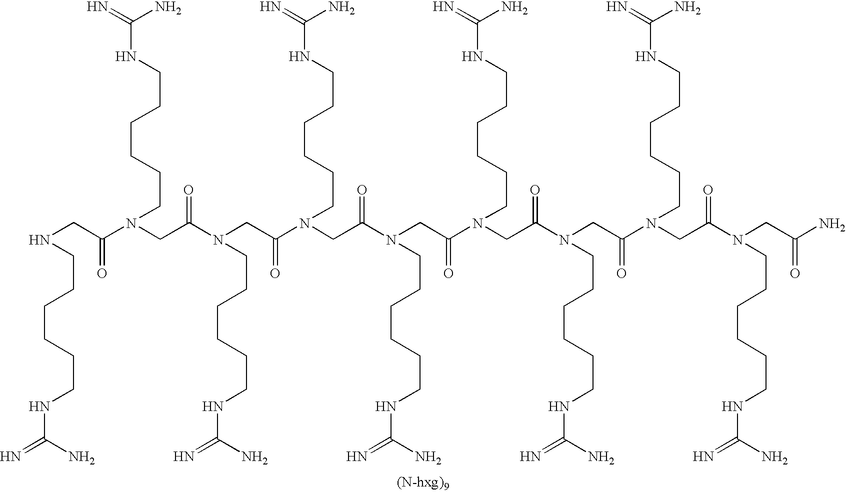

Additional charged bulky group include, without limitation, charged polypeptides, such as poly-arginine (guanidinium side chain), poly-lysine (ammonium side chain), poly-aspartic acid (carboxylate side chain), poly-glutamic acid (carboxlyate side chain), or poly-histidine (imidazolium side chain).

In certain embodiments, a charged polysaccharide (e.g., hyaluronic acid as shown below) may also be used.

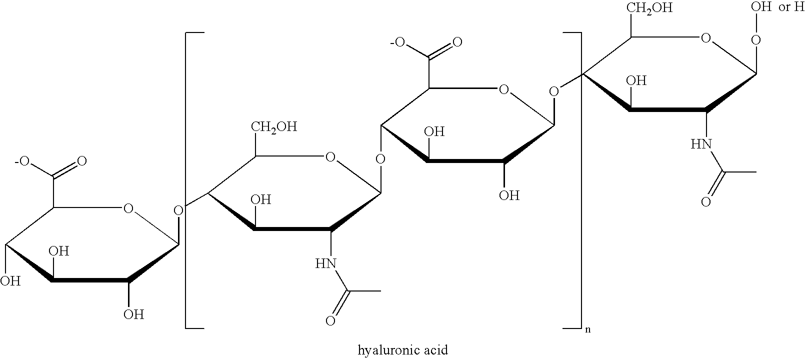

The bulky group can be an antiproliferative agent used in the combinations of the invention. Such conjugates are desirable where the two agents have matching pharmacokinetic profiles to enhance efficacy and/or to simplify the dosing regimen.

The bulky group may also include another therapeutic agent. Desirably, the therapeutic agent conjugated to the phenothiazine of formula (VII) via a linker of formula (XXXV) is a compound of formula (XXXVI):

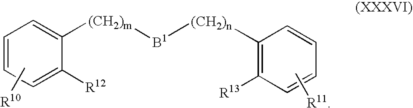

In formula (XXXVI), B¹ is selected from

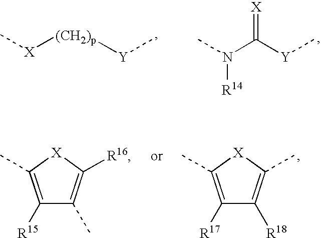

wherein each of X and Y is, independently, O, NR¹⁹, or S; each of R¹⁴ and R¹⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R¹⁵, R¹⁶, R¹⁷, and R¹⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R¹⁰ and R¹¹ is

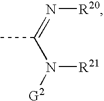

wherein R²¹ is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R²⁰ is H, OH, or acyl, or R²⁰ and R²¹ together represent

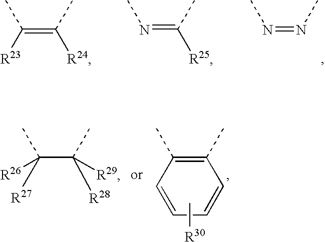

wherein each of R²³, R²⁴, and R²⁵ is, independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R²⁶, R²⁷, R²⁸, and R²⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R¹² and R¹³ is, independently, H, Cl, Br, OH, OCH₃, OCF₃, NO₂, and NH₂, or R¹² and R¹³ together form a single bond; and G² is a bond between the compound of formula (XXVI) and the linker.

Antiproliferatives that can be conjugates to a phenothiazine compound include pentamidine, shown below, as well as 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis [5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis {p-[2-(3,4,5,6-tetrahydropyrimidyl)phenyl]}furan, 2,5-bis[4-(2-imidazolinyl)phenyl]furan, 2,5 [bis- {4-(2-tetrahydropyrimidinyl)}phenyl]-3-(p-tolyloxy)furan, 2,5 [bis {4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1 H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis {4-[3-(dimethylaminopropyl)amidino]phenyl}furan, 2,5-bis {4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis {4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis {4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, or 2,5-bis[4-(N-(3-fluoro)phenoxycarbonyl)amidinophenyl]furan.

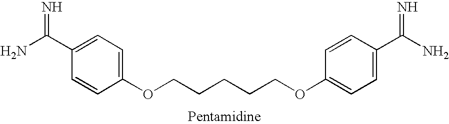

Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

The conjugate comprising, for example, a phenothiazine (A) and pentamidine (B), can be linked, without limitation, as dimers, trimers, or tetramers, as shown below. 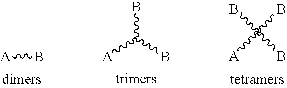
Charged Groups

By “charged group” is meant a group comprising three or more charged moieties.

By “charged moiety” is meant a moiety which loses a proton at physiological pH thereby becoming negatively charged (e.g., carboxylate, or phosphate), a moiety which gains a proton at physiological pH thereby becoming positively charged (e.g., ammonium, guanidinium, or amidinium), a moiety that includes a net formal positive charge without protonation (e.g., quaternary ammonium), or a moiety that includes a net formal negative charge without loss of a proton (e.g., borate, BR₄⁻).

In certain embodiments, charged groups are attached through the ring nitrogen of the phenothiazine.

A charged group may be cationic or an anionic. Charged groups include 3, 4, 5, 6, 7, 8, 9, 10, or more negatively charged moieties and/or 3, 4, 5, 6, 7, 8, 9, 10, or more positively charged moieties. Charged moieties include, without limitation, carboxylate, phosphodiester, phosphoramidate, borate, phosphate, phosphonate, phosphonate ester, sulfonate, sulfate, thiolate, phenolate, ammonium, amidinium, guanidinium, quaternary ammonium, and imidazolium moieties.

In certain embodiments, a charged group has a molecular weight less than 600, 400, 200, or 100 daltons.

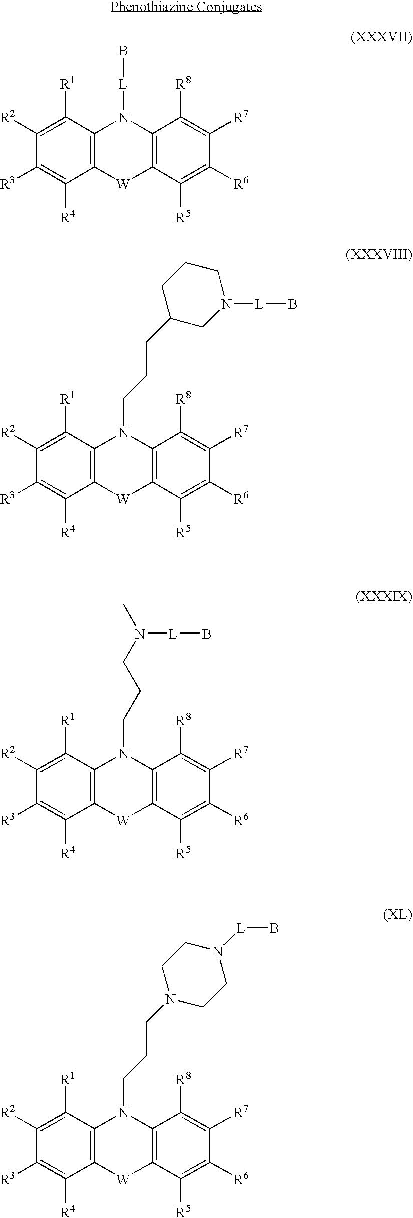

In formulas (XXXVII)-(XL), R¹, R², R³, R⁴, R⁵, R⁶, R⁷, R⁸, and W are as described above. L is a linker of formula (XXXV), described above. B is a bulky or charged group, as described above.

Methods for Preparing Exemplary Phenothiazine Conjugates

1. Protection and Deprotection of Reactive Groups

The synthesis of phenothiazine conjugates may involve the selective protection and deprotection of alcohols, amines, ketones, sulfhydryls or carboxyl functional groups of the phenothiazine, the linker, the bulky group, and/or the charged group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulthydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994.

In the examples that follow, the use of protecting groups is indicated in a structure by the letter P, where P for any amine, aldehyde, ketone, carboxyl, sulfhydryl, or alcohol may be any of the protecting groups listed above.

2. Polyguanidine Conjugates of Phenothiazines

2-(trifluoromethyl)phenothiazine (CAS 92-30-8, Aldrich Cat. No. T6,345-2) can be reacted with an activated carboxyl. Carboxyls can be activated, for example, by formation of an active ester, such as nitrophenylesters, N-hydroxysuccinimidyl esters, or others as described in Chem. Soc. Rev. 12:129, 1983 and Angew. Chem. Int. Ed. Engl. 17:569, 1978, incorporated herein by reference. For example, oxalic acid (Aldrich, catalogue number 24,117-2) can be attached as a linking group, as shown below in reaction

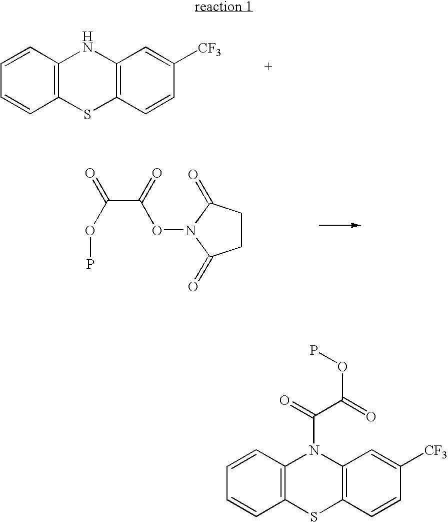

The protecting group in the reaction product can be removed by hydrolysis. The resulting acid is available for conjugation to a bulky group or a charged group.

The polyguanidine peptoid N-hxg, shown below, can be prepared according to the methods described by Wender et al., Proc. Natl. Acad. Sci. USA 97(24):13003-8, 2000, incorporated herein by reference.

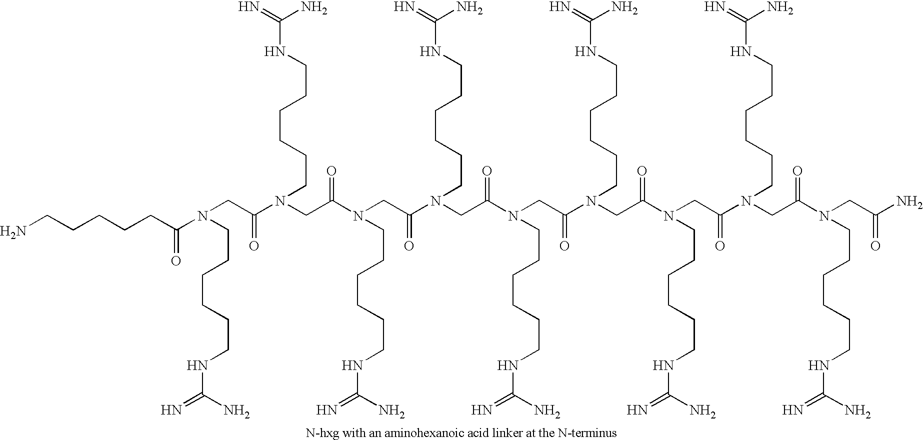

The carboxyl derivative produced by the deprotection of the product of reaction 1 can be activated, vide supra, and conjugated to the protected precursor of N-hxg followed by the formation of the guanidine moieties and cleavage from the solid phase resin, as described by Wender ibid., to produce the polyguanidine prednisolone conjugate shown below.

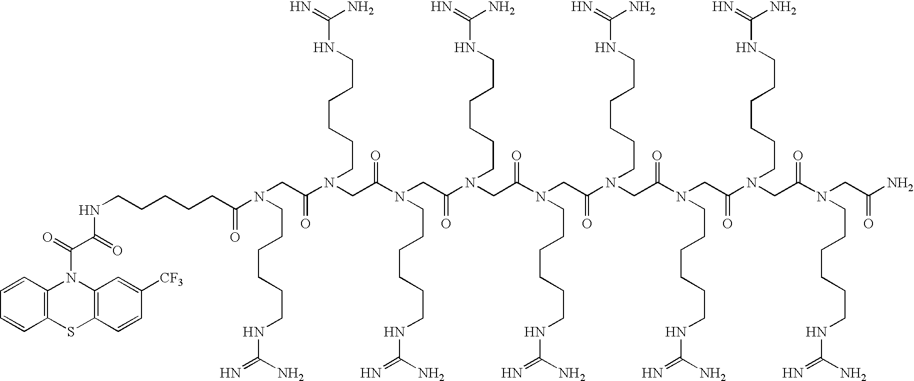

The resulting phenothiazine conjugate includes a bulky group (FW 1,900 Da) which includes several positively charged moieties.

3. Hyaluronic Acid Conjugates of a Phenothiazines

2-Methylthiophenothiazine (CAS 7643-08-5, Aldrich Cat. No. 55,292-5) can be reacted a hydrazine-substituted carboxylic acid, which has been activated as shown in reaction 3.

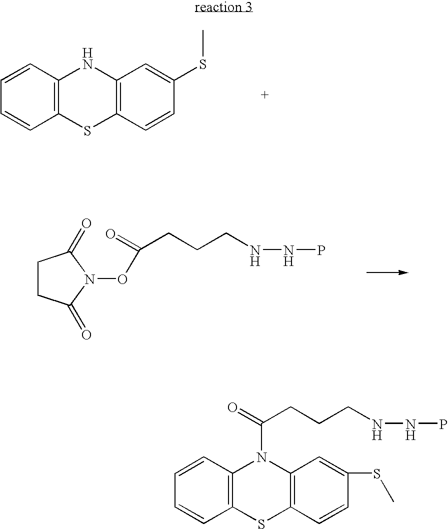

The protecting group can be removed from the reaction product and the free hydrazine coupled to a carboxyl group of hyaluronic acid as described by, for example, Vercruysse et al., Bioconjugate Chem., 8:686, 1997 or Pouyani et al., J. Am. Chem. Soc., 116:7515, 1994. The structure of the resulting hydrazide conjugate is provided below:

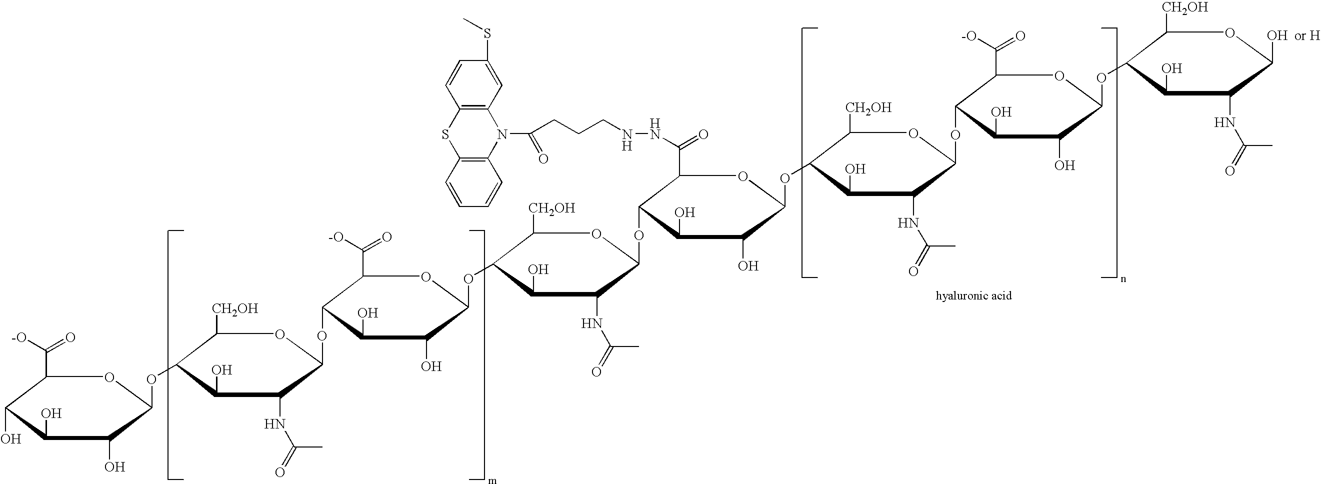

In the phenothiazine conjugate above, the hyaluronic acid is approximately 160,000 Daltons in molecular weight. Accordingly, m and n are whole integers between 0 and 400. Conjugates of lower and higher molecular weight hyaluronic acid can be prepared in a similar fashion.

4. PEG Conjugates of Phenothiazines

(10-piperadinylpropyl)phenothiazine can be conjugated to mono-methyl polyethylene glycol 5,000 propionic acid N-succinimidyl ester (Fluka, product number 85969). The resulting mPEG conjugate, shown below, is an example of a phenothiazine conjugate of a bulky uncharged group.

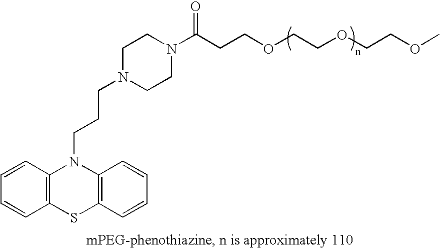

Conjugates of lower and higher molecular weight mPEG can be prepared in a similar fashion (see, for example, Roberts et al., Adv. Drug Delivery Rev. 54:459 (2002)).

Chlorpromazine can be conjugated to an activated PEG (e.g., a mesylate, or halogenated PEG compound) as shown in reaction 4.

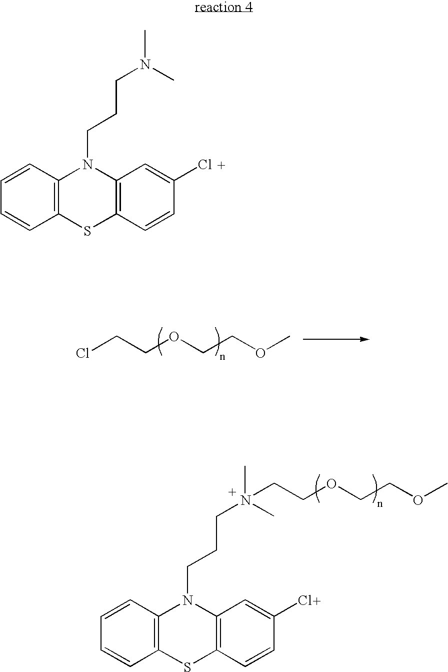

5. Pentamidine Conjugates of Phenothiazines

Pentamadine conjugates of phenothiazine can be prepared using a variety of conjugation techniques. For example, reaction 5 shows perimethazine, the alcohol activated in situ (e.g., using mesylchloride), followed by alkylation of pentamidine to form the conjugate product of the two therpeutic agents. 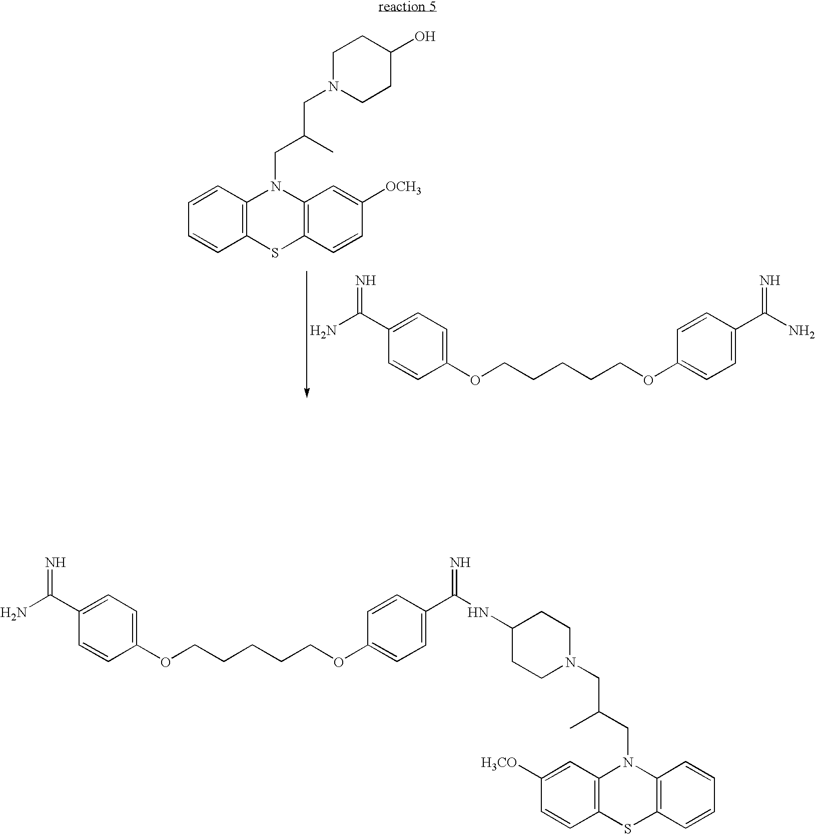
Combinations Comprising Phenothiazines and Antiproliferative Agents

In another aspect, the drug combinations may comprise (a) a compound of formula (XLI):

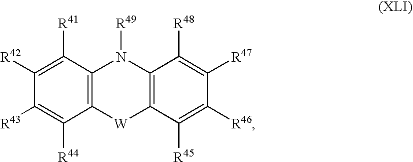

or a pharmaceutically active or acceptable salt thereof, wherein R⁴² is selected from the group consisting of: CF₃, halogen, OCH₃, COCH₃, CN, OCF₃, COCH₂CH₃, CO(CH₂)₂CH₃, S(O)₂CH₃, S(O)₂N(CH₃)₂, and SCH₂CH₃;

R⁴⁹ is selected from the group consisting of:

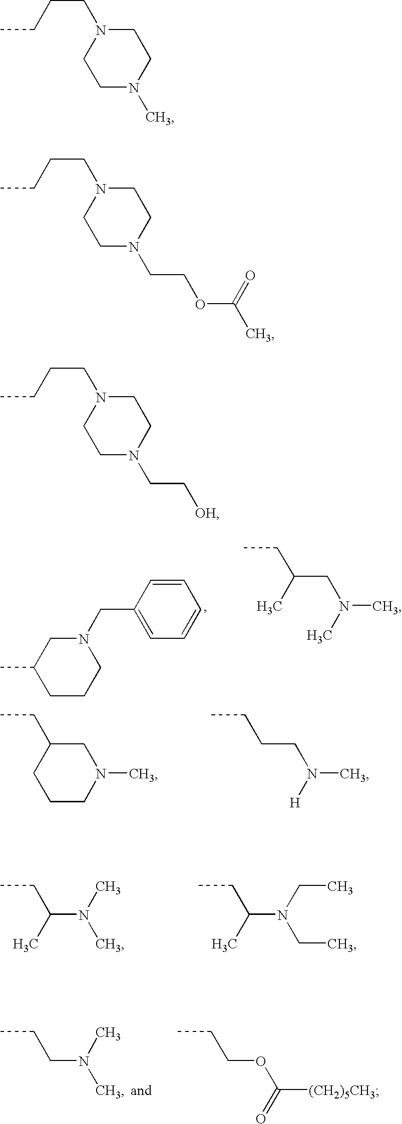

each of R⁴¹, R⁴³, R⁴⁴, R⁴⁵, R⁴⁶, R⁴⁷, and R⁴⁸ is independently H, OH, F, OCF₃, or OCH₃; and W is selected from the group consisting of: NO,

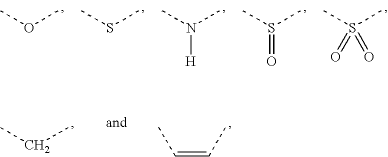

(b) an antiproliferative agent, wherein each are present in amounts that together are sufficient to inhibit the growth of a neoplasm.

Preferably, the compound of formula (XLI) is acepromazine, chlorpromazine, cyamemazine, fluphenazine, mepazine, methotrimeprazine, methoxypromazine, perazine, perphenazine, prochlorperazine, promethazine, propiomazine, thiethylperazine, thiopropazate, thioridazine, trifluoperazine, or triflupromazine.

Antiproliferative agents are described above, such as those in Tables 1 and 2.

In certain embodiments, the drug combination contains an anti-proliferative agent of formula (XLII):

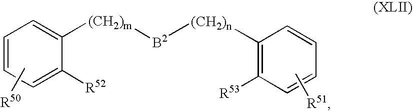

or a pharmaceutically active or acceptable salt thereof. In formula (XLII), B² is

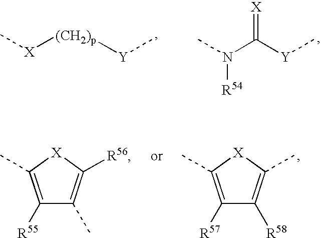

wherein each of X and Y is, independently, O, NR⁵⁹, or S; each of R⁵⁴ and R⁵⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R⁵⁵, R⁵⁶, R⁵⁷, and R⁵⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R⁵⁰ and R⁵¹ is

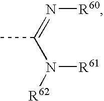

wherein R⁶¹ is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R⁶² is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, alkoxy, aryloxy, or C₁₋₇ heteroalkyl; and R⁶⁰ is H, OH, or acyl, or R⁶⁰ and R⁶¹ together represent

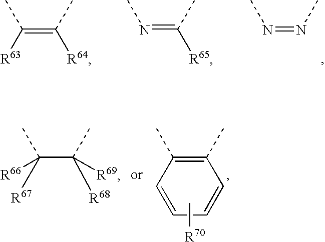

wherein each of R⁶³, R^{64,} and R⁶⁵ is, independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R⁶⁶, R⁶⁷, R⁶⁸, and R⁶⁹ is, independently, H. C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R⁵² and R⁵³ is, independently, H, Cl, Br, OH, OCH₃, OCF₃, NO₂, and NH₂, or R⁵² and R⁵³ together form a single bond.

Compounds of formula (XLII) useful in the methods and compositions of the invention include pentamidine, propamidine, butamidine, heptamidine, nonamidine, stilbamidine, hydroxystilbamidine, diminazene, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, and 2,4-bis(4-amidinophenyl)thiophene-bis-O-methylamidoxime.

In one embodiment, the compound of formula (XLI) is chlorpromazine, perphenazine or promethazine and the compound of formula (XLII) is pentamidine, 2,5-bis(4-amidinophenyl)furan, or 2,5-bis(4-amidinophenyl)furan-bis-O-methylamidoxime.

The invention also features a drug combination that includes (a) a first compound selected from prochlorperazine, perphenazine, mepazine, methotrimeprazine, acepromazine, thiopropazate, perazine, propiomazine, putaperazine, thiethylperazine, methopromazine, chlorfenethazine, cyamemazine, perphenazine, norchlorpromazine, trifluoperazine, thioridazine (or a salt of any of the above), and dopamine D2 antagonists (e.g., sulpride, pimozide, spiperone, ethopropazine, clebopride, bupropion, and haloperidol), and, (b) a second compound selected from pentamidine, propamidine, butamidine, heptamidine, nonamidine, stilbamidine, hydroxystilbamidine, diminazene, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5[bis {4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3 [(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis [4(1-acetoxyethoxycarbonyl)amidinophenyl]furan, and 2,5-bis[4-(N-(3-fluoro)phenoxycarbonyl)amidinophenyl]furan, or a salt of any of the above.

Alternatively, the second compound can be a functional analog of pentamidine, such as netropsin, distamycin, bleomycin, actinomycin, daunorubicin, or a compound that falls within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1.

Combinations Comprising Kinesin Inhibitors and Antiproliferative Agents

In certain embodiments, the drug combinations of the present invention may comprise kinesin inhibitors and antiproliferative agents (e.g., Group A and Group B antiproliferative agents).

Kinesin Inhibitors

By “kinesin inhibitor” is meant a compound that inhibits by a statistically significant amount (e.g., by at least 10%, 20%, 30%, or more) the enzymatic activity of a mitotic kinesin (e.g., HsEg5). Mitotic kinesins are enzymes essential for assembly and function of the mitotic spindle and play essential roles during all phases of mitosis. Perturbation of mitotic kinesin function causes malformation or dysfunction of the mitotic spindle, frequently resulting in cell cycle arrest and cell death. Kinesin inhibitors can be identified using a variety of methods as disclosed in PCT publication WO02/057244. For example, kinesin inhibition can be identified using assays for cell cycle distribution, cell viability, morphology, activity, or by monitoring the formation of mitotic spindles.

Methods for monitoring cell cycle distribution of a cell population include, for example, flow cytometry. Kinesin inhibitors include, without limitation, chlorpromazine, monasterol, terpendole E, HR²²C16, and SB715992. Other mitotic kinesin inhibitors are those compounds disclosed in Hopkins et al., Biochemistry 39:2805, 2000, Hotha et al., Angew Chem. Inst. Ed. 42:2379, 2003, PCT Publication Nos. WO01/98278, WO02/057244, WO02/079169, WO02/057244, WO02/056880, WO03/050122, WO03/050064, WO03/049679, WO03/049678, WO03/049527, WO03/079973, and WO03/039460; U.S. Patent Application Publication Nos. 2002/0165240, 2003/0008888, 2003/0127621, and 2002/0143026; and U.S. Pat. Nos., 6,437,115, 6,545,004, 6,562,831, 6,569,853, and 6,630,479.

In certain embodiments, the kinesin inhibitors are phenothiazines, analogs or metabolites. Such compounds are described above in the sections related to combinations comprising chlorpromazine and pentamidine and to combinations comprising phenothiazine conjugates or phenothiazines and antiproliferative agents.

In certain embodiments, the kinesin inhibitor may be a compound having the formula (XLIII): 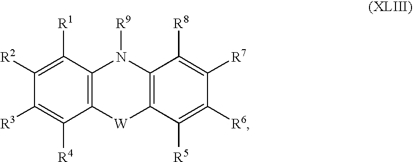
or a pharmaceutically acceptable salt thereof,

wherein R² is CF₃, halogen, OCH₃, COCH₃, CN, OCF₃, COCH₂CH₃, CO(CH₂)₂CH₃, or SCH₂CH₃;

R⁹ is selected from:

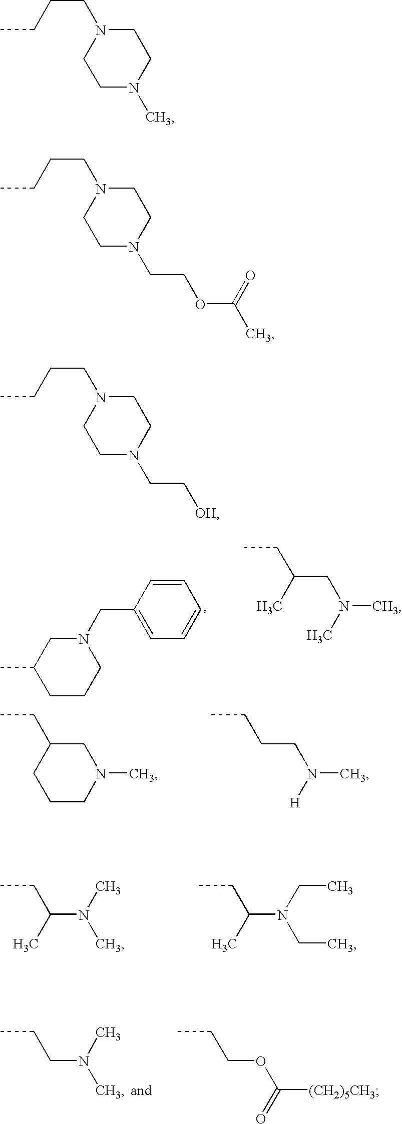

or R⁹ has the formula: 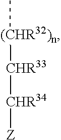
wherein n is 0 or 1, Z is NR³⁵R³⁶ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;

each of R¹, R³, R⁴, R⁵, R⁶, R⁷, and R⁸ is independently H, OH, F, OCF₃, or OCH₃; and

W is NO,

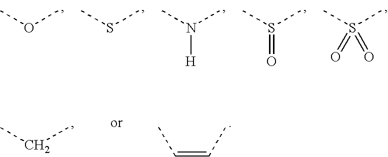

Exemplary kinesin inhibitors include acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine.

Antiproliferative Agents

Antiproliferative agents are described above. In certain embodiments, antiproliferative agents are Group A antiproliferative agents (e.g., those listed in Table 1). In certain embodiments, the antiproliferative agents are not pentamidines or their analogs, endo-exonuclase inhibitors, PRL phosphatase inhibitors, or PTP1B inhibitors.

In certain embodiments, Group A antiproliferative agents may be an alkylating agent (e.g., dacarbazine), an anthracycline (e.g., mitoxantrone), an anti-estrogen (e.g., bicalutamide), an anti-metabolite (e.g., floxuridine), a microtubule binding, stabilizing agent (e.g., docetaxel), microtubule binding, destabilizing agent (e.g., vinorelbine), topoisomerase inhibitor (e.g., hydroxycamptothecin (SN-38)), or a kinase inhibitor (e.g., a tyrphostin, such as AG1478). In certain embodiments, the agent is altretamine, carmustine, chlorambucil, cyclophosphamide, dacarbazine, ifosfamide, melphalan, mitomycin, temozolomide, doxorubicin, epirubicin, mitoxantrone, anastrazole, bicalutamide, estramustine, exemestane, flutamide, fulvestrant, tamoxifen, toremifene, capecitabine, floxuridine, fluorouracil, gemcitabine, hydroxyurea, methotrexate, gleevec, tyrphostin, docetaxel, pacilitaxel, vinblastine, vinorelbine, adjuvant/enhancing agents (celecoxib, gallium, isotretinoin, leucovorin, levamisole, pamidronate, suramin), or agents such as thalidomide, carboplatin, cisplatin, oxaliplatin, etoposide, hydroxycamptothecin, irinotecan, or topotecan. In certain other embodiments, the Group A antiproliferative agent is selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, or rituximab, or any combination thereof.

In certain embodiments, the antiproliferative agent may be a bis-benzimidazole compound.

By “bis-benzimidazole compound” is meant a compound of formula (XLIV):

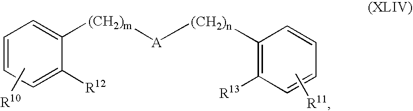

wherein A is selected from:

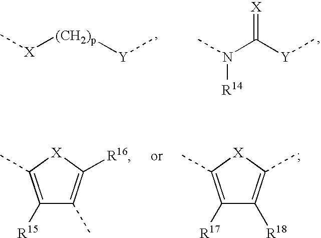

each of X and Y is, independently, O, NR¹⁹, or S; each of R¹⁴ and R¹⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R¹⁵, R¹⁶, R¹⁷, and R¹⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R¹⁰ and R¹¹ is

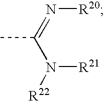

each of R²¹ and R²² is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R²⁰ is H, OH, or acyl, or R²⁰ and R²¹ together represent

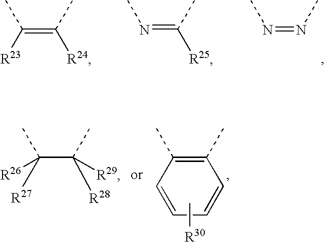

each of R²³, R²⁴, and R²⁵ is, independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R²⁶, R²⁷, R²⁸, and R²⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R¹² and R¹³ is, independently, H, Cl, Br, OH, OCH₃, OCF₃, NO₂, and NH₂, or R¹² and R¹³ together form a single bond. Bis-benzimidazole compounds include pentamidine, propamidine, butamidine, heptamidine, nonamidine, stilbamidine, hydroxystilbamidine, diminazene, berenil, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5[bis-{4-(2-tetrahydropyrimidinyl)}phenyl]-3-(p-tolyloxy)furan, 2,5[bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis [5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis [5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-l-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a salt of any of the above. Bis-benzimidazole compounds also include functional analogs of pentamidine, such as netropsin, distamycin, bleomycin, actinomycin, daunorubicin. Bis-benzimidazole compounds further include any compound that falls within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and any compound that falls within a formula provided in any of U.S. Patent Application Publication Nos. US 2001/0044468 A1 and US 2002/0019437 A1. Bis-benzimidazole compounds include any compound identified as a pentamidine analog, or falling within a formula that includes pentamidine, provided in U.S. Pat. No. 6,569,853 and in U.S. Patent Application Publication No. 20040116407 A1.

Exemplary Drug Combinations

In certain embodiments, the drug combinations of the present invention comprise (1) a kinesin inhibitor selected from acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine, and (2) a Group A antiproliferative agent selected from dacarbazine, mitoxantrone, bicalutamide, floxuridine, leucovorin, vinblastine, vinorelbine, hydroxycamptothecin, tyrphostin, docetaxel, or combinations thereof.

In certain other embodiments, the drug combinations of the present invention comprises (1) a kinesin inhibitor selected from acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine, and (2) a Group A antiproliferative agent selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, rituximab, or combinations thereof.

In certain embodiments, when the drug combinations comprise trifluoperazine, the antiproliferative agents in the combinations are not doxorubicin, aclacinomycin, trifluoroacetyladriamycin-14-valerate, vinblastine, dactinomycin, colchicine, or adriamycin.

In certain other embodiments, when the drug combinations comprise chlorpromazine, the antiproliferative agents in the combinations are not paclitaxel, doxorubicin, vinblastine, dactinomycin, or colchicines.

In certain other embodiments, when the drug combinations comprise thioridazine, the antiproliferative agents in the combinations are not doxorubicin, vinblastine, dactinomycin, or colchicine.

In certain embodiments, the drug combinations of the present invention comprise chlorpromazine and dacarbazine, chlorpromazine and floxuridine, chlorpromazine and tyrphostin 1486, chlorpromazine and vinblastine, chlorprmazine and hydroxycamptothecin, chlorpromazine and leucovorin, chlorpromazine and paclitaxel, or chlorpromazine and docetaxel.

Combinations Comprising Mitotic Kinesin Inhibitors and Protein Tyrosine Phosphatase Inhibitors

In certain embodiments, the drug combinations of the present invention comprise agents that reduce the biological activity of a mitotic kinesin and agents that reduce the biological activity of protein tyrosine phosphatases. In certain embodiments, the drug combinations further comprise one or more antiproliferative agents.

Mitotic Kinesins

Mitotic kinesins are essential motors in mitosis. They control spindle assembly and maintenance, attachment and proper positioning of the chromosomes to the spindle, establish the bipolar spindle and maintain forces in the spindle to allow movement of chromosomes toward opposite poles. Perturbations of mitotic kinesin function cause malformation or dysfunction of the mitotic spindle, frequently resulting in cell cycle arrest and cell death.

Exemplary mitotic kinesins include HsEg5/KSP, KIFC3, CHO2, MKLP, MCAK, Kin2, Kif4, MPP1, CENP-E, NYREN62, LOC8464, and KIF8. Other mitotic kinesins are described in U.S. Pat. Nos. 6,414,121, 6,582,958, 6,544,766, 6,492,158, 6,455,293, 6,440,731, 6,437,115, 6,420,162, 6,399,346, 6,395,540, 6,383,796, 6,379,941, and 6,248,594. The GenBank Accession Nos. of representative mitotic kinesins are provided below.

Human mitotic kinesins

| Protein name | GenBank Accession No. |
|---|---|
| Eg5/KSP | AA857025, U37426, X85137 |
| KIFC3 | BC001211 |
| MKLP1 | AI131325, AU133373, X67155 |
| MCAK | AL046197, U63743 |
| KIN2 | Y08319 |
| KIF4 | AF071592 |
| MPP1 | AL117496 |
| CENP-E | Z15005 |
| CHO2 | AL021366 |
| HsNYREN62 | AF155117 |
| HsLOC8464 | NM_032559 |
| KIF8 | AB001436 |

HsEg5/KSP has been cloned and characterized (see, e.g., Blangy et al., Cell, 83:1159-69 (1995); Galgio et al., J. Cell Biol., 135:399-414, 1996; Whitehead et al., J. Cell Sci., 111:2551-2561, 1998; Kaiser, et al., J. Biol. Chem., 274:18925-31, 1999; GenBank accession numbers: X85137, NM 004523). Drosophila (Heck et al., J. Cell Biol., 123:665-79, 1993) and Xenopus (Le Guellec et al., Mol. Cell Biol., 11:3395-8, 1991) homologs of KSP have been reported. Drosophila KLP61F/KRP130 has reportedly been purified in native form (Cole, et al., J. Biol. Chem., 269:22913-22916, 1994), expressed in E. coli, (Barton, et al., Mol. Biol. Cell, 6:1563-74, 1995) and reported to have motility and ATPase activities (Cole, et al., supra; Barton, et al., supra). Xenopus Eg5/KSP was expressed in E. coli and reported to possess motility activity (Sawin, et al., Nature, 359:540-3, 1992; Lockhart and Cross, Biochemistry, 35:2365-73, 1996; Crevel, et al., J. Mol. Biol., 273:160-170, 1997) and ATPase activity (Lockhart and Cross, supra; Crevel et al., supra).

Besides KSP, other members of the BimC family include BimC, CIN8, cut7, KIP1, KLP61F (Barton et al., Mol. Biol. Cell. 6:1563-1574, 1995; Cottingham & Hoyt, J. Cell Biol. 138:1041-1053, 1997; DeZwaan et al., J. Cell Biol. 138:1023-1040, 1997; Gaglio et al., J. Cell Biol. 135:399-414, 1996; Geiser et al., Mol. Biol. Cell 8:1035-1050, 1997; Heck et al., J. Cell Biol. 123:665-679, 1993; Hoyt et al., J. Cell Biol. 118:109-120, 1992; Hoyt et al., Genetics 135:35-44, 1993; Huyett et al., J. Cell Sci. 111:295-301, 1998; Miller et al., Mol. Biol. Cell 9:2051-2068, 1998; Roof et al., J. Cell Biol. 118:95-108, 1992; Sanders et al., J. Cell Biol. 137:417-431, 1997; Sanders et al., Mol. Biol. Cell 8:1025-0133, 1997; Sanders et al., J. Cell Biol. 128:617-624, 1995; Sanders & Hoyt, Cell 70:451-458, 1992; Sharp et al., J. Cell Biol. 144:125-138, 1999; Straight et al., J. Cell Biol. 143:687-694, 1998; Whitehead & Rattner, J. Cell Sci. 111:2551-2561, 1998; Wilson et al., J. Cell Sci. 110:451-464, 1997).

Mitotic kinesin biological activities include its ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods for assaying biological activity of a mitotic kinesin are well known in the art. For example, methods of performing motility assays are described, e.g., in Hall, et al., 1996, Biophys. J., 71:3467-3476, Turner et al, 1996, Anal. Biochem. 242:20-25; Gittes et al., 1996, Biophys. J. 70:418-429; Shirakawa et al., 1995, J. Exp. Biol. 198: 1809-1815; Winkelmann et al., 1995, Biophys. J. 68: 2444-2453; and Winkelmann et al., 1995, Biophys. J. 68:72S. Methods known in the art for determining ATPase hydrolysis activity also can be used. U.S. application Ser. No. 09/314,464, filed May 18, 1999, hereby incorporated by reference in its entirety, describes such assays. Other methods can also be used. For example, Pᵢ release from kinesin can be quantified. In one embodiment, the ATP hydrolysis activity assay utilizes 0.3 M perchloric acid (PCA) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to nM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to nM Pᵢ and plotted over time. Additionally, ATPase assays known in the art include the luciferase assay.

ATPase activity of kinesin motor domains also can be used to monitor the effects of modulating agents. In one embodiment ATPase assays of kinesin are performed in the absence of microtubules. In another embodiment, the ATPase assays are performed in the presence of microtubules. Different types of modulating agents can be detected in the above assays. In one embodiment, the effect of a modulating agent is independent of the concentration of microtubules and ATP. In another embodiment, the effect of the agents on kinesin ATPase may be decreased by increasing the concentrations of ATP, microtubules, or both. In yet another embodiment, the effect of the modulating agent is increased by increasing concentrations of ATP, microtubules, or both.

Agents that reduce the biological activity of a mitotic kinesin in vitro may then be screened in vivo. Methods for in vivo screening include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability (see, e.g., U.S. Pat. No. 6,617,115).

Mitotic Kinesin Inhibitors

By “mitotic kinesin inhibitor” is meant an agent that binds a mitotic kinesin and reduces, by a significant amount (e.g., by at least 10%, 20% 30% or more), the biological activity of that mitotic kinesin. Mitotic kinesin biological activities include enzymatic activity (e.g., ATPase activity), motor activity (e.g., generation of force) and binding activity (e.g., binding of the motor to either microtubules or its cargo).

Mitotic kinesin inhibitors include chlorpromazine, monasterol, terpendole E, HR22C16, and SB715992. Other mitotic kinesin inhibitors are those compounds disclosed in Hopkins et al., Biochemistry 39:2805, 2000, Hotha et al., Angew Chem. Inst. Ed. 42:2379, 2003, PCT Publication Nos. WO01/98278, WO02/057244, WO02/079169, WO02/057244, WO02/056880, WO03/050122, WO03/050064, WO03/049679, WO03/049678, WO03/049527, WO03/079973, and WO03/039460, and U.S. Patent Application Publication Nos. 2002/0165240, 2003/0008888, 2003/0127621, and 2002/0143026; and U.S. Pat. Nos., 6,437,115, 6,545,004, 6,562,831, 6,569,853, and 6,630,479, and the chlorpromazine analogs described in U.S. patent application Ser. No. 10/617,424, which are also described above.

Protein Tyrosine Phosphatases

Protein tyrosine phosphatases (PTPases) are intracellular signaling molecules that dephosphorylate a tyrosine residue on a protein substrate, thereby modulating certain cellular functions. In normal cells, they typically act in concert with protein tyrosine kinases to regulate signaling cascades through the phosphorylation of protein tyrosine residues. Phosphorylation and dephosphorylation of the tyrosine residues on proteins controls cell growth and proliferation, cell cycle progression, cytoskeletal integrity, differentiation and metabolism. In various metastatic and cancer cell lines, PTP1B and the family of Phosphatases of Regenerating Liver (PRL-1, PRL-2, and PRL-3) have been shown to be overexpressed. For example, PRL-3 (also known as PTP4A3) is expressed in relatively high levels in metatstatic colorectal cancers (Saha et al., Science 294: 1343-1346, 2001.). PRL-1 localizes to the mitotic spindle and is required for mitotic progression and chromosome segregation. PRL phosphatases promote cell migration, invasion, and metastasis, and inhibition of these PTPases has been shown to inhibit proliferation of cancer cells in vitro and tumors in animal models.

By “protein tyrosine phosphatase” or “PTPase” is meant an enzyme that dephosphorylates a tyrosine residue on a protein substrate.

By “dual specificity phosphatase” is meant a protein phosphatase that can dephosphorylate both a tyrosine residue and either a serine or threonine residue on the same protein substrate. Dual specificity phosphatases include

MKP-1, MKP-2, and the cell division cycle phosphatase family (e.g., CDC14, CDC25A, CDC25B, and CDC25C). Dual specificity phosphatases are considered to be protein tyrosine phosphatases.

Protein tyrosine phosphatases include the PRL family (PRL-1, PRL-2, and PRL-3), PTP1B, SHP-1, SHP-2, MKP-1, MKP-2, CDC14, CDC25A, CDC25B, CDC25C, PTPα, and PTP-BL. Protein tyrosine phosphatase biological activities include dephosphorylation of tyrosine residues on substrates. The GenBank Accession Nos. of representative tyrosine phosphatases are provided below.

| Protein | |
|---|---|
| name | GenBank Accession No. |
| PRL-1 | AJ420505, BI222469, U48296 |
| PRL-2 | AF208850, BI552091, L48723 |
| PRL-3 | AF041434, BC003105 |
| PTP1B | AU117677, M33689 |
| SHP-1 | BC002523, BG754792, M77273, BM742181, AF178946 |
| SHP-2 | AU123593, BF515187, BX537632, D13540 |
| MKP-1 | U01669, X68277 |
| MKP-2 | BC014565, U21108, U48807, AL137704 |
| CDC14A | AF000367, AF064102, AF064103 |
| CDC14B | AF023158, AF064104 |
| CDC25A | M81933 |
| CDC25B | M81934, Z68092, AF036233 |
| CDC25C | M34065, Z29077, AJ304504, M34065 |
| PTPalpha | M36033 |
| PTP-BL | D21210, D21209, D21211, U12128 |

Protein Tyrosine Phosphatase Inhibitors

By “protein tyrosine phosphatase inhibitor” is an agent that binds a protein tyrosine phosphatase and inhibits (e.g., by at least 10%, 20%, or 30% or more) the biological activity of that protein tyrosine phosphatase.

Inhibitors of protein tyrosine phosphatases include pentamidine, levamisole, ketoconazole, bisperoxovanadium compounds (e.g., those described in Scrivens et al., Mol. Cancer Ther. 2:1053-1059, 2003, and U.S. Pat. No. 6,642,221), vanadate salts and complexes (e.g., sodium orthovanadate), dephosphatin, dnacin A1, dnacin A2, STI-571, suramin, gallium nitrate, sodium stibogluconate, meglumine antimonate, 2-(2-mercaptoethanol)-3-methyl-1,4-naphthoquinone, 2,5-bis(4-amidinophenyl)furan-bis-O-methylamidoxime, known as DB289 (Immtech), 2,5-bis(4-amidinophenyl)furan (DB75, Immtech), disclosed in U.S. Pat. No. 5,843,980, and compounds described in Pestell et al., Oncogene 19:6607-6612, 2000, Lyon et al., Nat. Rev. Drug Discov. 1:961-976, 2002, Ducruet et al., Bioorg. Med. Chem. 8:1451-1466, 2000, U.S. Patent Application Publication Nos. 2003/0114703, 2003/0144338, and 2003/0161893, and PCT Patent Publication Nos. WO099/46237, WO03/06788 and WO03/070158. Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and U.S. Patent Application Publication Nos. US 2001/0044468 and US 2002/0019437, and the pentamidine analogs described in U.S. patent application Ser. No. 10/617,424 (see, e.g., Formula (II)). Other protein tyrosine phosphatase inhibitors can be identified, for example, using the methods described in Lazo et al. (Oncol. Res. 13:347-352, 2003), PCT Publication Nos. WO097/40379, WO03/003001, and WO03/035621, and U.S. Pat. Nos. 5,443,962 and 5,958,719.

Other biological activity inhibitors

In addition to reducing biological activity through the use of compounds that bind a mitotic kinesin or protein tyrosine phosphatase, other inhibitors of mitotic kinesin and protein tyrosine phosphatase biological activity can be employed. Such inhibitors include compounds that reduce the amount of target protein or RNA levels and compounds that compete with endogenous mitotic kinesins or protein tyrosine phosphatases for binding partners (e.g., dominant negative proteins).

Dominant Negative Proteins

By “dominant negative” is meant a protein that contains at least one mutation that inactivates its physiological activity such that the expression of this mutant in the presence of the normal or wild type copy of the protein results in inactivation of or reduction of the activity of the normal copy. Thus, the activity of the mutant “dominates” over the activity of the normal copy such that even though the normal copy is present, biological function is reduced. In one example, a dimer of two copies of the protein are required so that even if one normal and one mutated copy are present there is no activity; another example is when the mutant binds to or “soaks up” other proteins that are critical for the function of the normal copy such that not enough of these other proteins are present for activity of the normal copy.

One skilled in the art would know how to make dominant negative mitotic kinesins and protein tyrosine phosphatases. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992.

Aurora Kinase Inhibitors

Aurora kinases have been shown to be protein kinases of a new family that regulate the structure and function of the mitotic spindle. One target of Aurora kinases include mitotic kinesins. Aurora kinase inhibitors thus can be used in combination with a compound that reduces protein tyrosine phosphatase biological activity according to a method, composition, or kit of the invention.

There are three classes of aurora kinases: aurora-A, aurora-B and aurora-C. Aurora-A includes AIRK1, DmAurora, HsAurora-2, HsAIK, HsSTK15, CeAIR-1, MmARK1, MmAYK1, MmIAK1 and XIEg2. Aurora-B includes AIRK-2, DmIAL-1, HsAurora-1, HsAIK2, HsAIM-1, HsSTK12, CeAIR-2, MmARK2 and XAIRK2. Aurora-C includes HsAIK3 (Adams, et al., Trends Cell Biol. 11:49-54, 2001).

Aurora kinase inhibitors include VX-528 and ZM447439; others are described, e.g., in U.S. Patent Application Publication No. 2003/0105090 and U.S. Pat. Nos. 6,610,677, 6,593,357, and 6,528,509.

Farnesyltransferase Inhibitors

Farnesyltransferase inhibitors alter the biological activity of PRL phosphatases and thus can be used in combination with a compound that reduces mitotic kinesin activity in a method, composition, or kit of the invention. Farnesyltransferase inhibitors include arglabin, lonafarnib, BAY-43-9006, tipifarnib, perillyl alcohol, FTI-277 and BMS-214662, as well as those compounds described, e.g., in Kohl, Ann. NY Acad. Sci. 886:91-102, 1999, U.S. Patent Application Publication Nos. 2003/0199544, 2003/0199542, 2003/0087940, 2002/0086884, 2002/0049327, and 2002/0019527, U.S. Pat. Nos. 6,586,461 and 6,500,841, and WO03/004489.

Antiproliferative Agents

Antiproliferative agents are described above. Exemplary antiproliferative agents of the invention include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors.

Pharmaceutical Compositions

The present invention, in another aspect, provides pharmaceutical compositions that comprise an anti-scarring drug combination. In certain embodiments, the pharmaceutical compositions further comprise a polymer, a secondary agent (e.g., an anti-infective agent, an anti-inflammatory agent or an anti-thrombotic agent), a pharmaceutical excipient, and/or an agent that facilitates the delivery of the anti-scarring drug combination or compositions.

Compositions that Comprise Anti-infective Agents

The compositions useful in the present invention may also include anti-infective agents. Such agents may reduce the likelihood of infection upon implantation of the composition or a medical implant and may be used in combination of an anti-fibrosis drug combination (or individual component(s) thereof) and/or a polymer.

Infection is a common complication of the implantation of foreign bodies such as, for example, medical devices and implants. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases. In many cases, an infected implant or device must be surgically removed from the body to irradicate the infection.

The present invention provides agents (e.g., chemotherapeutic agents) that can be released from a composition, and which have potent antimicrobial activity at extremely low doses. A wide variety of anti-infective agents can be utilized in combination with the present compositions. Suitable anti-infective agents may be readily determined based upon the assays provided in Example 30). Discussed in more detail below are several representative examples of agents that can be used as anti-infective agents, such as: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

Anthracyclines

In certain embodiments, the therapeutic anti-infective agent is an anthracycline. Anthracyclines have the following general structure, where the R groups may be a variety of organic groups:

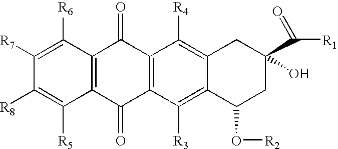

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, ydroxyl, or methoxy; and R₆₋₈ are all hydrogen. Alternatively, R₅ and R₆ are hydrogen and R₇ and R₈ are alkyl or halogen, or vice versa. According to U.S. Pat. No. 5,843,903, R₁ may be a conjugated peptide.

According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)-R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 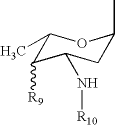
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

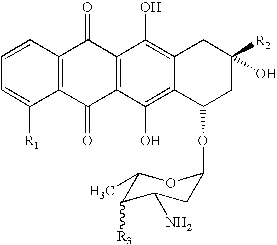

| | R₁ | R₂ | R₃ |
|---|---|---|---|
| Doxorubicin: | OCH₃ | C(O)CH₂OH | OH |
| | | | out of ring plane |
| Epirubicin: | OCH₃ | C(O)CH₂OH | OH |
| (4′ epimer of | | | in ring plane |
| doxorubicin) | | | |
| Daunorubicin: | OCH₃ | C(O)CH₃ | OH |
| | | | out of ring plane |
| Idarubicin: | H | C(O)CH₃ | OH |
| | | | out of ring plane |
| Pirarubicin: | OCH₃ | C(O)CH₂OH | 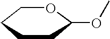 |
| Zorubicin: | OCH₃ | C(CH₃)(═N)NHC(O)C₆H₅ | OH |
| Carubicin: | OH | C(O)CH₃ | OH |
| | | | out of ring plane |

Other suitable anthracyclines are anthramycin, mitoxantrone, menogaril, nogalamycin, aclacinomycin A, olivomycin A, chromomycin A₃, and plicamycin having the structures:

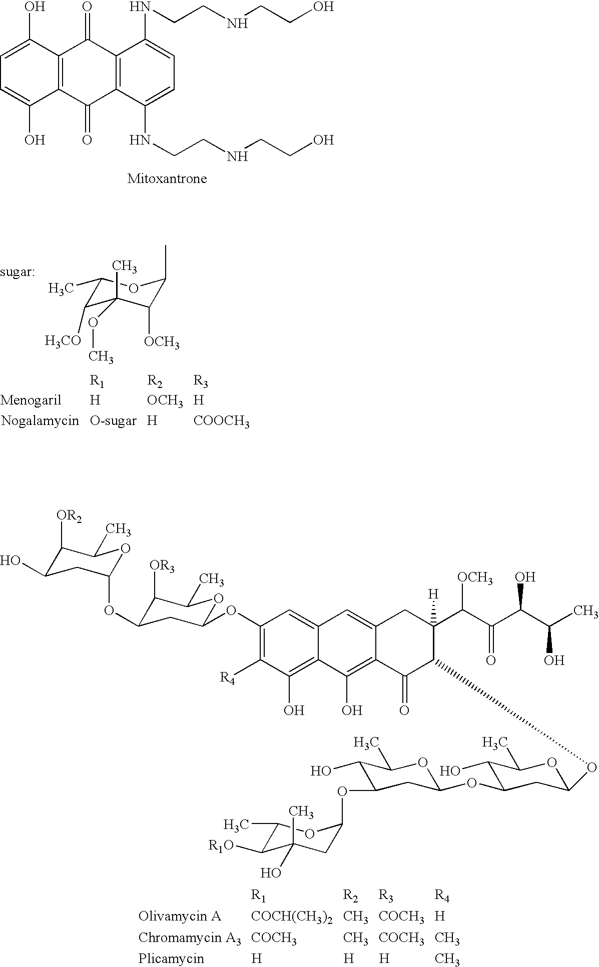

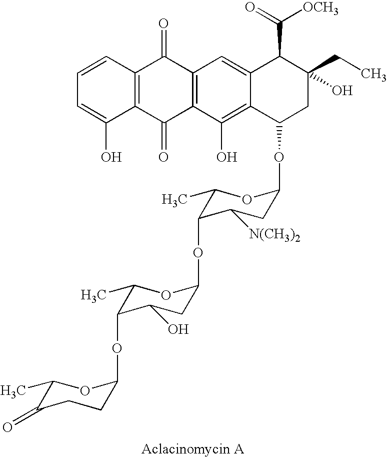

Other representative anthracyclines include, FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6): 1109-1116, 1998), 2-pyrrolinodoxorubicin (Nagy et al., Proc. Nat'l Acad Sci. U.S.A. 95(4):1794-1799, 1998), disaccharide doxorubicin analogues (Arcamone et al., J. Nat'l Cancer Inst. 89(16): 1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)adriamicinone doxorubicin disaccharide analogue (Monteagudo et al., Carbohydr. Res. 300(1):11-16, 1997), 2-pyrrolinodoxorubicin (Nagy et al., Proc. Nat'l Acad Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl)doxorubicin derivatives (U.S. Pat. No. 4,314,054), 3′-deamino-3′-(4-mortholinyl)doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl)doxorubicin derivatives (U.S. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl)daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No.4,585,859), 3′-deamino-3′-(4-methoxy-1-piperidinyl)doxorubicin derivatives (U.S. Pat. No.4,314,054) and 3-deamino-3-(4-morpholinyl) doxorubicin derivatives (U.S. Pat. No. 4,301,277).

Fluoropyrimidine Analogues

In other embodiments, the ant-infective agent is a fluoropyrimidine analog, such as 5-fluorouracil, or an analogue or derivative thereof, including carmofur, doxifluridine, emitefur, tegafur, and floxuridine. Exemplary compounds have the structures:

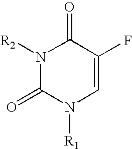

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

B

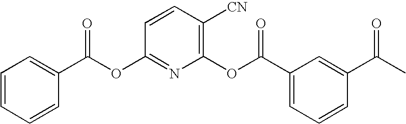

C

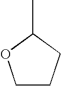

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures: 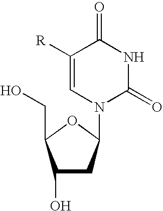
5-Fluoro-2′-deoxyuridine: R═F
5-Bromo-2′-deoxyuridine: R═Br
5-Iodo-2′-deoxyuridine: R═I

Other representative examples of fluoropyrimidine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4): 1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

Folic Acid Antagonists

In certain embodiments, the anti-infective agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure: 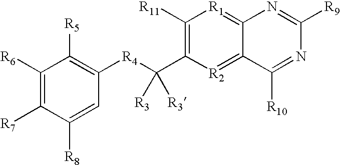
The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 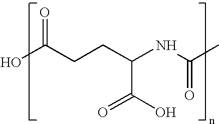
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

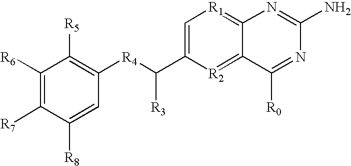

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetraxate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Peritrexim | NH₂ | N | C(CH₃) | H | single bond | OCH₃ | H | H | OCH₃ |

A:

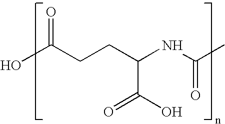

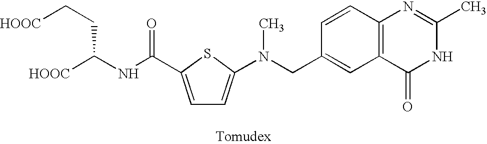

Other representative examples include 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaaminopterin analogues (DeGraw et al., J. Med. Chem. 40(3):370-376, 1997), 5-deazaaminopterin and 5,10-dideazaaminopterin methotrexate analogues (Piper et al., J. Med Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm., Biopharm. Pharm. Technol., 563-4, 1995), L-threo-(2S,4S)-4-fluoroglutamic acid and DL-3,3-difluoroglutamic acid-containing methotrexate analogues (Hart et al., J. Med Chem. 39(1):56-65, 1996), methotrexate tetrahydroquinazoline analogue (Gangjee, et al., J. Heterocycl Chem. 32(1):243-8, 1995), N-(α-aminoacyl)methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β, γ-methano methotrexate analogues (Rosowsky et al., Pteridines 2(3):133-9, 1991), 10-deazaaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl)methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(A dv. Biomed. Polym.):311-24, 1987), methotrexate-y-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly (L-lysine)methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N. Y. Acad. Sci. 186:J227-34, 1971), MX068 (Pharma Japan, 1658:18, 1999) and cysteic acid and homocysteic acid methotrexate analogues (EPA 0142220).

These compounds are believed to act as antimetabolites of folic acid.

Podophyllotoxins

In certain embodiments, the anti-infective therapeutic agent is a Podophyllotoxin, or a derivative or an analogue thereof. Exemplary compounds of this type are etoposide or teniposide, which have the following structures:

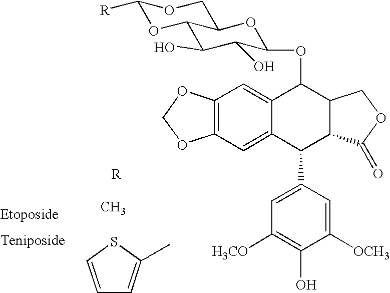

Other representative examples of podophyllotoxins include Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med Chem. 37(2):287-92, 1994), N-glucosyl etoposide analogue (Allevi et al., Tetrahedron Lett. 34(45):7313-16, 1993), etoposide A-ring analogues (Kadow et al., Bioorg Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

Camptothecins

In certain embodiments, the anti-infective therapeutic agent is camptothecin, or an analogue or derivative thereof. Camptothecins have the following general structure.

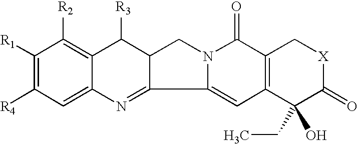

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

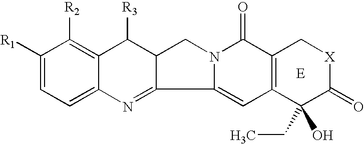

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

Hydroxyureas

The anti-infective therapeutic agent of the present invention may be a hydroxyurea. Hydroxyureas have the following general structure:

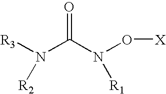

Suitable hydroxyureas are disclosed in, for example, U.S. Pat. No. 6,080,874, wherein R₁ is: 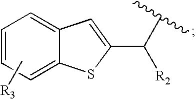
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 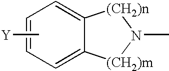
where in m is 1 or 2, n is 0-2 and Y is an alkyl group.

In one aspect, the hydroxyurea has the structure:

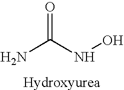

These compounds are thought to function by inhibiting DNA synthesis.

Platinum Complexes

In certain embodiments, the anti-infective therapeutic agent is a platinum compound. In general, suitable platinum complexes may be of Pt(II) or Pt(IV) and have this basic structure:

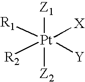

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

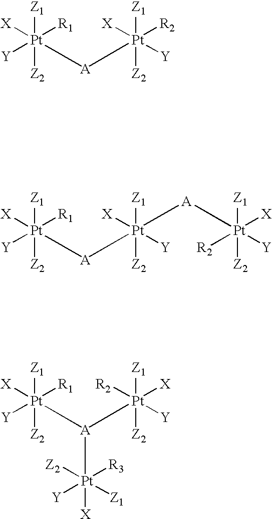

Exemplary platinum compounds are cisplatin, carboplatin, oxaliplatin, and miboplatin having the structures:

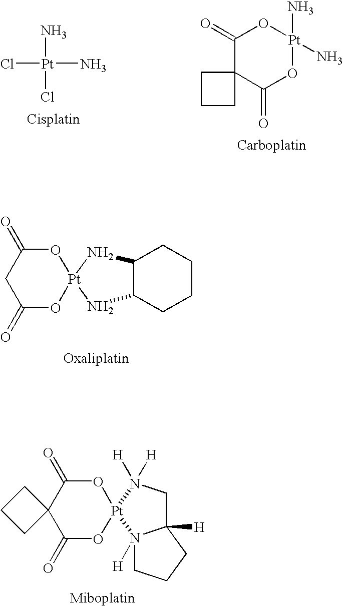

Other representative platinum compounds include (CPA)₂Pt(DOLYM) and (DACH)Pt(DOLYM) cisplatin (Choi et al., Arch. Pharmacal Res. 22(2):151-156, 1999), Cis-(PtCl₂(4,7-H-5-methyl-7-oxo)1,2,4(triazolo(1,5-a)pyrimidine)₂) (Navarro et al., J. Med. Chem. 41(3):332-338, 1998), (Pt(cis-1,4-DACH)(trans-Cl₂)(CBDCA)).1/2 MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂(NHCHN(C(CH₂)(CH₃)))₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-(Pt(OAc)₂I₂(en)) (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-(Pt(NH₃)(4-aminoTEMP-O){d(GpG)}) (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bemays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine)dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum(II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), and cis-dichloro(amino acid)(tert-butylamine)platinum(II) complexes (Pasini & Bersanetti, Inorg. Chim. Acta 107(4):259-67, 1985). These compounds are thought to function by binding to DNA, i.e., acting as alkylating agents of DNA.

Other Anti-Infective Agents

In certain embodiments, the anti-infective therapeutic agent is a quinolone antibacterial agent. Representative examples of quinolone antibacterial agents include garenoxacin (Schering Plough) or an analogue or derivative thereof.

Dosages of Anti-Infective Agents

The drug dose administered from the present compositions for prevention or inhibition of infection will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

(a) Anthracyclines. Utilizing the anthracycline doxorubicin as an example, whether applied as a polymer coating, incorporated into the polymers which make up the implant components, or applied without a carrier polymer, the total dose of doxorubicin applied to the device or implant should not exceed 25 mg (range of 0.1 μg to mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.0 μg-100 μg per mm² of surface area. In a particularly preferred embodiment, doxorubicin should be applied to the implant surface at a dose of 0.1 μg/mm²-10 μg/mm². As different polymer and non-polymer coatings will release doxorubicin at differing rates, the above dosing parameters should be utilized in combination with the release rate of the drug from the implant surface such that a minimum concentration of 10⁻⁷-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the device or implant, or applied without a carrier polymer, the total dose of mitoxantrone applied should not exceed 5 mg (range of 0.01 μg to 5 mg). In a particularly preferred embodiment, the total amount of drug applied should be in the range of 0.1 μg to 1 mg. The dose per unit area (i.e., the amount of drug as a function of the surface area of the portion of the implant to which drug is applied and/or incorporated) should fall within the range of 0.01 μg -20 μg per mm² of surface area. In a particularly preferred embodiment, mitoxantrone should be applied to the implant surface at a dose of 0.05 μg/mm²-3 μg/mm². As different polymer and non-polymer coatings will release mitoxantrone at differing rates, the above dosing parameters should be utilized in combination with the release rate of the drug from the implant surface such that a minimum concentration of 10⁻⁵-10⁻⁶ M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines. Utilizing the fluoropyrimidine 5-fluorouracil as an example, whether applied as a polymer coating, incorporated into the polymers which make up the device or implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.1 μg-1 mg per mm² of surface area. In a particularly preferred embodiment, 5-fluorouracil should be applied to the implant surface at a dose of 1.0 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins. Utilizing the podophylotoxin etoposide as an example, whether applied as a polymer coating, incorporated into the polymers which make up the device or implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 10 μg/mm²-10 μg/mm². As different polymer and non-polymer coatings will release etoposide at differing rates, the above dosing parameters should be utilized in combination with the release rate of the drug from the implant surface such that a concentration of 10⁻⁵-10⁻⁶ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), podophylotoxins (e.g., etoposide), and/or quinolones can be utilized to enhance the antibacterial activity of the composition.

Compositions That Comprise Polymers

In certain embodiments, the compositions of the present invention may comprise a polymer that facilitates the delivery of an anti-scarring drug combination (or individual component(s) thereof) or forms a sustained release formuation for an anti-scarring drug combination (or individual component(s) thereof). In certain embodiments, compositions that comprise polymers may further comprise additional agents (e.g., secondary agents, pharmaceutical exicipents, echogenic agents, etc.).

For instance, the composition may be or include a hydrophilic polymer gel that has anti-thrombogenic properties. Such a composition can be in the form of a coating that can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Sustained-Release Preparations of Anti-Scarring Drug Combinations or Individual Components

In certain embodiments, desired anti-scarring drug combinations or individual components of the combinations may be admixed with, blended with, conjugated to, or, otherwise modified to contain a polymer composition (which may be either biodegradable or non-biodegradable) or a non-polymeric composition in order to release the drug combination or individual components thereof over a prolonged period of time. For many of the aforementioned embodiments, localized delivery as well as localized sustained delivery of the fibrosis-inhibiting drug combination or individual component(s) thereof may be required. For example, a desired anti-scarring drug combination or individual component(s) thereof may be admixed with, blended with, conjugated to, or, otherwise modified to contain a polymeric composition (which may be either biodegradable or non-biodegradable) or non-polymeric composition in order to release the anti-scarring drug combination or individual component(s) thereof over a period of time.

Representative examples of biodegradable polymers suitable for the delivery of the aforementioned anti-scarring drug combination or individual component(s) thereof include albumin, collagen, gelatin, hyaluronic acid, starch, cellulose and cellulose derivatives (e.g., regenerated cellulose, methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, polyesters, poly(malic acid), poly(tartronic acid), poly(acrylamides), polyanhydrides, polyphosphazenes, poly(amino acids), poly(alkylene oxide)-poly(ester) block copolymers (e.g., X-Y, X-Y-X, Y-X-Y, R-(Y-X)ₙ, or R-(X-Y)ₙ, where X is a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLA, PCL, polydioxanone and copolymers thereof) and R is a multifunctional initiator, n is an integer, preferably from 2 to 12). The compositions can also include blends of the above polymers as well as copolymers thereof. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

Representative examples of non-degradable polymers suitable for the delivery of a fibrosis-inhibiting anti-scarring drug combination or individual component(s) thereof include poly(ethylene-co-vinyl acetate) (“EVA”) copolymers, non-degradable polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate)poly(hexylcyanoacrylate)poly(octylcyanoacrylate)), acrylic resin, polyethylene, polypropylene, polyamides (nylon 6,6), polyurethanes (e.g., CHRONOFLEX AR, CHRONOFLEX AL, BIONATE, and PELLETHANE), poly(ester urethanes), poly(ether urethanes), poly(ester-urea), cellulose esters (e.g., nitrocellulose), polyethers (poly(ethylene oxide), poly(propylene oxide), polyoxyalkylene ether block copolymers based on ethylene oxide and propylene oxide such as the PLURONIC polymers (e.g., F-127 or F87) from BASF Corporation (Mount Olive, N.J.), and poly(tetramethylene glycol), styrene-based polymers (polystyrene, poly(styrene sulfonic acid), poly(styrene)-block-poly(isobutylene)-block-poly(styrene), poly(styrene)-poly(isoprene) block copolymers), and vinyl polymers(polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends, copolymers and branched polymers thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995).

Some examples of preferred polymeric carriers include poly(ethylene-co-vinyl acetate), polyurethanes (e.g., CHRONOFLEX AR, CHRONOFLEX AL, BIONATE, and PELLETHANE), poly (D,L-lactic acid)oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), poly(alkylene oxide)-poly(ester) block copolymers (e.g., X-Y, X-Y-X or Y-X-Y, R-(Y-X)ₙ, R-(X-Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.), R is a multifunctional initiator, n is an integer, preferably from 2 to 12, and copolymers as well as blends thereof), nitrocellulose, silicone rubbers, poly(styrene) block-poly(isobutylene) block-poly(styrene), poly(acrylate)polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers, as well as blends thereof.

Other representative polymers capable of sustained localized delivery of fibrosis-inhibiting drug combinations or individual components thereof include carboxylic polymers, polyacetates, polycarbonates, polyethers, polyethylenes, polyvinylbutyrals, polysilanes, polyureas, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, natural and synthetic elastomers, rubber, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as the corresponding U.S. applications), U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, 5,714,159, 5,612,052, and U.S. Patent Application Publication Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of anti-scarring drug combinations.

It should be also obvious to one of skill in the art that the polymers as described herein can also be blended or copolymerized in various compositions as required to deliver therapeutic doses of other pharmaceutically active agents (such as anti-infective agents).

Polymeric carriers for anti-scarring drug combination or individual component(s) thereof can be fashioned in a variety of forms, with desired release characteristics and/or with specific properties depending upon the composition being utilized. For example, polymeric carriers may be fashioned to release an anti-scarring drug combination or individual component(s) thereof upon exposure to a specific triggering event such as pH (see, e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Comejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly (acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide lmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, anti-scarring drug combinations or individual components thereof can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and the gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N,n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X-Y, Y-X-Y and X-Y-X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Anti-scarring drug combinations or individual components thereof may be linked by occlusion in the polymer matrix, dissolution in the polymer, bound by covalent linkages, bound by ionic interactions, or encapsulated in microcapsules. Within certain embodiments of the invention, therapeutic compositions are provided in non-capsular formulations such as microspheres (ranging from nanometers to micrometers in size), pastes, threads of various size, films, or sprays. In one aspect, the anti-scarring drug combination (or individual component(s) thereof) may be incorporated into biodegradable magnetic nanospheres. The nanospheres may be used, for example, to replenish an anti-scarring drug combination or individual component(s) thereof into an implanted intravascular device, such as a stent containing a weak magnetic alloy (see, e.g., Z. Forbes, B. B. Yellen, G. Friedman, K. Barbee. “An approach to targeted drug delivery based on uniform magnetic fields,” IEEE Trans. Magn. 39(5): 3372-3377 (2003)).

Within certain aspects of the present invention, therapeutic compositions of anti-scarring drug combinations may be fashioned in the form of microspheres, microparticles and/or nanoparticles having any size ranging from 50 nm to 500 μm, depending upon the particular use. These compositions can be formed by spray-drying methods, milling methods, coacervation methods, W/O emulsion methods, W/O/W emulsion methods, and solvent evaporation methods. In other aspects, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions that include anti-scarring drug combinations (or individual components thereof) may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing therapeutic agents are particularly useful for application to the surface of tissues that will be in contact with the implant or device.

Within yet other aspects of the invention, the therapeutic compositions of the present invention may be formed as a film or tube. These films or tubes can be porous or non-porous. Preferably, such films or tubes are generally less than 5, 4, 3, 2, or 1 mm thick, more preferably less than 0.75 mm, 0.5 mm, 0.25 mm, or, 0.10 mm thick. Films or tubes can also be generated of thicknesses less than 50 μm, 25 μm or 10 μm. Such films are preferably flexible with a good tensile strength (e.g., greater than 50, preferably greater than 100, and more preferably greater than 150 or 200 N/cm²), good adhesive properties (i.e., adheres to moist or wet surfaces), and have controlled permeability. Anti-scarring drug combinations or individual components thereof contained in polymeric films are particularly useful for application to the surface of a device or implant as well as to the surface of tissue, cavity or an organ.

Within further aspects of the present invention, polymeric carriers are provided which are adapted to contain and release a hydrophobic anti-scarring drug combination or individual component(s) thereof. In certain embodiments, the carriers that contains and release the hydrophobic drug combination or individual component(s) thereof are further in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic drug combination or individual component(s) thereof, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

The anti-scarring drug combinations or individual components thereof may be delivered as a solution. Such combinations or individual component(s) thereof can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethylcellulose (CMC), and the like). In another aspect of the invention, the solution can include a biocompatible solvent or liquid oligomers and/or polymers, such as ethanol, DMSO, glycerol, PEG-200, PEG-300 or NMP. These compositions may further comprise a polymer such a degradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, or block copolymers of the form X-Y, Y-X-Y, R-(Y-X)ₙ, R-(X-Y)ₙ and X-Y-X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG), R is a multifunctional initiator, and n is an integer, preferably from 2 to 12).

Within another aspect of the invention, compositions comprising anti-scarring drug combinations or individual components thereof can further comprise a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X-Y, Y-X-Y, R-(Y-X)ₙ, R-(X-Y)ₙ and X-Y-X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator), zeolites or cyclodextrins.

Other carriers that may likewise be utilized to contain and deliver anti-scarring drug combinations or individual components thereof described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid- aerosols, microemulsions (U.S. Pat. No. 5,330,756), polymeric shell (nano- and micro- capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977; U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975; U.S. Patent Application Publication Nos. 2002/012796A1, 2002/0127266A1, 2002/0151650A1, 2003/0104032A1, 2002/0091229A1, and 2003/0059906A1.

In certain aspects, it is desirable to use compositions that can be administered as liquids, but subsequently form hydrogels at the site of administration. Such in situ hydrogel forming compositions can be administered as liquids from a variety of different devices, and are more adaptable for administration to any site, since they are not preformed. Examples of in situ forming hydrogels include photoactivatable mixtures of water-soluble co-polyester prepolymers and polyethylene glycol to create hydrogel barriers. Block copolymers of polyalkylene oxide polymers (e.g., PLURONIC compounds from BASF Corporation, Mount Olive, N.J.) and poloxamers have been designed that are soluble in cold water, but form insoluble hydrogels that adhere to tissues at body temperature (Leach, et al., Am. J. Obstet. Gynecol. 162:1317-1319 (1990)).

In certain embodiments, the present invention provides for polymeric crosslinked matrices, and polymeric carriers, that may be used to assist in the prevention of the formation or growth of fibrous connective tissue. The composition may contain and deliver fibrosis-inhibiting drug combinations in the vicinity of the implanted device. The following compositions are particularly useful when it is desired to infiltrate around the device. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a delivery device (e.g., a syringe) in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

First and Second Synthetic Polymers

In one embodiment, crosslinked polymer compositions (in other words, crosslinked matrices) are prepared by reacting a first synthetic polymer containing two or more nucleophilic groups with a second synthetic polymer containing two or more electrophilic groups, where the electrophilic groups are capable of covalently binding with the nucleophilic groups. In one embodiment, the first and second polymers are each non-immunogenic. In another embodiment, the matrices are not susceptible to enzymatic cleavage by, e.g., a matrix metalloproteinase (e.g., collagenase) and are therefore expected to have greater long-term persistence in vivo than collagen-based compositions.

As used herein, the term “polymer” refers inter alia to polyalkyls, polyamino acids, polyalkyleneoxides and polysaccharides. Additionally, for external or oral use, the polymer may be polyacrylic acid or carbopol. As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing either nucleophilic or electrophilic groups are also referred to herein as “multifunctionally activated synthetic polymers.” The term “multifinctionally activated” (or, simply, “activated”) refers to synthetic polymers which have, or have been chemically modified to have, two or more nucleophilic or electrophilic groups which are capable of reacting with one another (i.e., the nucleophilic groups react with the electrophilic groups) to form covalent bonds. Types of multifunctionally activated synthetic polymers include difunctionally activated, tetrafunctionally activated, and star-branched polymers.

Multifinctionally activated synthetic polymers for use in the present invention must contain at least two, more preferably, at least three, functional groups in order to form a three-dimensional crosslinked network with synthetic polymers containing multiple nucleophilic groups (i.e., “multi-nucleophilic polymers”). In other words, they must be at least difunctionally activated, and are more preferably trifunctionally or tetrafunctionally activated. If the first synthetic polymer is a difunctionally activated synthetic polymer, the second synthetic polymer must contain three or more functional groups in order to obtain a three-dimensional crosslinked network. Most preferably, both the first and the second synthetic polymer contain at least three functional groups.

Synthetic polymers containing multiple nucleophilic groups are also referred to generically herein as “multi-nucleophilic polymers.” For use in the present invention, multi-nucleophilic polymers must contain at least two, more preferably, at least three, nucleophilic groups. If a synthetic polymer containing only two nucleophilic groups is used, a synthetic polymer containing three or more electrophilic groups must be used in order to obtain a three-dimensional crosslinked network.

Preferred multi-nucleophilic polymers for use in the compositions and methods of the present invention include synthetic polymers that contain, or have been modified to contain, multiple nucleophilic groups such as primary amino groups and thiol groups. Preferred multi-nucleophilic polymers include: (i) synthetic polypeptides that have been synthesized to contain two or more primary amino groups or thiol groups; and (ii) polyethylene glycols that have been modified to contain two or more primary amino groups or thiol groups. In general, reaction of a thiol group with an electrophilic group tends to proceed more slowly than reaction of a primary amino group with an electrophilic group.

In one embodiment, the multi-nucleophilic polypeptide is a synthetic polypeptide that has been synthesized to incorporate amino acid residues containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000.

Poly(lysine)s for use in the present invention preferably have a molecular weight within the range of about 1,000 to about 300,000; more preferably, within the range of about 5,000 to about 100,000; most preferably, within the range of about 8,000 to about 15,000. Poly(lysine)s of varying molecular weights are commercially available from Peninsula Laboratories, Inc. (Belmont, Calif.) and Aldrich Chemical (Milwaukee, Wis.).

Polyethylene glycol can be chemically modified to contain multiple primary amino or thiol groups according to methods set forth, for example, in Chapter 22 of Poly(ethylene Glycol) Chemistry: Biotechnical and Biomedical Applications, J. Milton Harris, ed., Plenum Press, N.Y. (1992). Polyethylene glycols which have been modified to contain two or more primary amino groups are referred to herein as “multi-amino PEGs.” Polyethylene glycols which have been modified to contain two or more thiol groups are referred to herein as “multi-thiol PEGs.” As used herein, the term “polyethylene glycol(s)” includes modified and or derivatized polyethylene glycol(s).

Various forms of multi-amino PEG are commercially available from Shearwater Polymers (Huntsville, Ala.) and from Huntsman Chemical Company (Utah) under the name “Jeffamine.” Multi-amino PEGs useful in the present invention include Huntsman's Jeffamine diamines (“D” series) and triamines (“T” series), which contain two and three primary amino groups per molecule, respectively.

Polyamines such as ethylenediamine (H₂N—CH₂—CH₂—NH₂), tetramethylenediamine (H₂N—(CH₂)₄—NH₂), pentamethylenediamine (cadaverine) (H₂N—(CH₂)₅—NH₂), hexamethylenediamine (H₂N—(CH₂)₆—NH₂), di(2-aminoethyl)amine (HN—(CH₂CH₂—NH₂)₂), and tris(2-aminoethyl)amine (N—(CH₂—CH₂—NH₂)₃) may also be used as the synthetic polymer containing multiple nucleophilic groups.

Synthetic polymers containing multiple electrophilic groups are also referred to herein as “multi-electrophilic polymers.” For use in the present invention, the multifunctionally activated synthetic polymers must contain at least two, more preferably, at least three, electrophilic groups in order to form a three-dimensional crosslinked network with multi-nucleophilic polymers. Preferred multi-electrophilic polymers for use in the compositions of the invention are polymers which contain two or more succinimidyl groups capable of forming covalent bonds with nucleophilic groups on other molecules. Succinimidyl groups are highly reactive with materials containing primary amino (NH₂) groups, such as multi-amino PEG, poly(lysine), or collagen. Succinimidyl groups are slightly less reactive with materials containing thiol (SH) groups, such as multi-thiol PEG or synthetic polypeptides containing multiple cysteine residues.

As used herein, the term “containing two or more succinimidyl groups” is meant to encompass polymers which are preferably commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

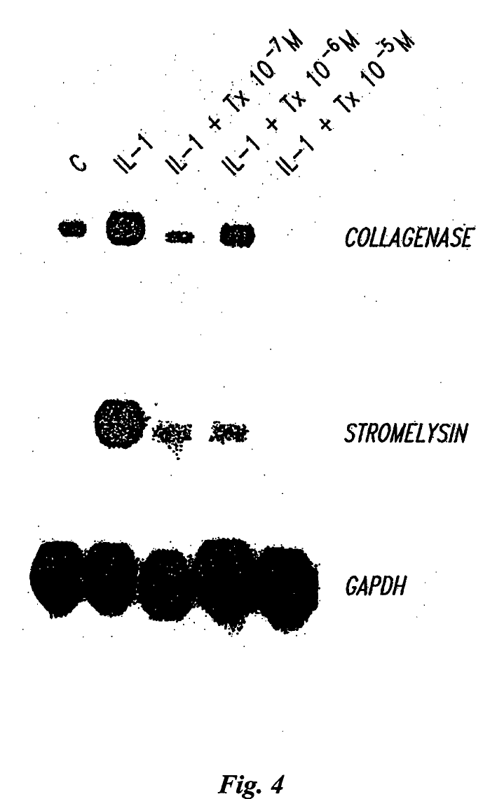

Hydrophilic polymers and, in particular, various derivatized polyethylene glycols, are preferred for use in the compositions of the present invention. As used herein, the term “PEG” refers to polymers having the repeating structure (OCH₂—CH₂)ₙ. Structures for some specific, tetrafunctionally activated forms of PEG are shown in FIGS. 4 to 13 of U.S. Pat. No. 5,874,500, incorporated herein by reference. Examples of suitable PEGS include PEG succinimidyl propionate (SE-PEG), PEG succinimidyl succinamide (SSA-PEG), and PEG succinimidyl carbonate (SC-PEG). In one aspect of the invention, the crosslinked matrix is formed in situ by reacting pentaerythritol poly(ethylene glycol)ether tetra-sulfhydryl](4-armed thiol PEG) and pentaerythritol poly(ethylene glycol)ether tetra-succinimidyl glutarate](4-armed NHS PEG) as reactive reagents. Structures for these reactants are shown in U.S. Pat. No. 5,874,500. Each of these materials has a core with a structure that may be seen by adding ethylene oxide-derived residues to each of the hydroxyl groups in pentaerythritol, and then derivatizing the terminal hydroxyl groups (derived from the ethylene oxide) to contain either thiol groups (so as to form 4-armed thiol PEG) or N-hydroxysuccinimydyl groups (so as to form 4-armed NHS PEG), optionally with a linker group present between the ethylene oxide derived backbone and the reactive functional group, where this product is commercially available as COSEAL from Angiotech Pharmaceuticals Inc. Optionally, a group “D” may be present in one or both of these molecules, as discussed in more detail below.

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propionaldehyde (A-PEG), or functionally activated PEG glycidyl ether (E-PEG), or functionally activated PEG-isocyanate (I-PEG), or functionally activated PEG-vinylsulfone (V-PEG).

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers which contain a relatively small proportion of oxygen or nitrogen atoms.

Hydrophobic polymers which already contain two or more succinimidyl groups include, without limitation, disuccinimidyl suberate (DSS), bis(sulfosuccinimidyl) suberate (BS3), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives. The above-referenced polymers are commercially available from Pierce (Rockford, Ill.), under catalog Nos. 21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.). According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—(CH₂)₆—COOH), and hexadecanedioic acid (HOOC—(CH₂)₁₄—COOH) are derivatized by the addition of succinimidyl groups to produce difunctionally activated polymers.

Polyamines such as ethylenediamine, tetramethylenediamine, pentamethylenediamine (cadaverine), hexamethylenediamine, bis (2-aminoethyl)amine, and tris(2-aminoethyl)amine can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

In a preferred embodiment, the first synthetic polymer will contain multiple nucleophilic groups (represented below as “X”) and it will react with the second synthetic polymer containing multiple electrophilic groups (represented below as “Y”), resulting in a covalently bound polymer network, as follows:
Polymer-Xₘ+Polymer-Yₙ→Polymer-Z-Polymer

wherein m≦2, n≦2, and m+n≦5;

where exemplary X groups include —NH₂, —SH, —OH, —PH₂, CO—NH—NH₂, etc., where the X groups may be the same or different in polymer-Xₘ;

where exemplary Y groups include —CO₂—N(COCH₂)₂, —CO₂H, —CHO, —CHOCH₂ (epoxide), —N═C═O, —SO₂—CH═CH₂, —N(COCH)₂ (i.e., a five-membered heterocyclic ring with a double bond present between the two CH groups), —S—S—(C₅H₄N), etc., where the Y groups may be the same or different in polymer-Yₙ; and

where Z is the functional group resulting from the union of a nucleophilic group (X) and an electrophilic group (Y).

As noted above, it is also contemplated by the present invention that X and Y may be the same or different, i.e., a synthetic polymer may have two different electrophilic groups, or two different nucleophilic groups, such as with glutathione.

In one embodiment, the backbone of at least one of the synthetic polymers comprises alkylene oxide residues, e.g., residues from ethylene oxide, propylene oxide, and mixtures thereof. The term ‘backbone’ refers to a significant portion of the polymer.

For example, the synthetic polymer containing alkylene oxide residues may be described by the formula X-polymer-X or Y-polymer-Y, wherein X and Y are as defined above, and the term “polymer” represents —(CH₂CH₂ O)ₙ— or —(CH(CH₃)CH₂ O)ₙ—or —(CH₂—CH₂—O)ₙ—(CH(CH₃)CH₂—O)ₙ—. In these cases the synthetic polymer would be difunctional.

The required functional group X or Y is commonly coupled to the polymer backbone by a linking group (represented below as “Q”), many of which are known or possible. There are many ways to prepare the various functionalized polymers, some of which are listed below:
Polymer-Q₁-X+Polymer-Q₂-Y→Polymer-Q₁-Z-Q₂-Polymer

Exemplary Q groups include —O—(CH₂)ₙ—; —S—(CH₂)ₙ—; —NH—(CH₂)ₙ—; —O₂C—NH—(CH₂)ₙ—; —O₂C—(CH₂)ₙ—; —O₂C—(CR¹H)ₙ—; and —O—R₂—CO—NH—, which provide synthetic polymers of the partial structures: polymer-O—(CH₂)ₙ—(X or Y); polymer-S—(CH₂)ₙ—(X or Y); polymer-NH—(CH₂)ₙ—(X or Y); polymer-O₂C—NH—(CH₂)ₙ—(X or Y); polymer-O₂C—(CH₂)ₙ—(X or Y); polymer-O₂C—(CR¹H)ₙ—(X or Y); and polymer-O—R₂—CO—NH—(X or Y), respectively. In these structures, n=1-10, R¹=H or alkyl (i.e., CH₃, C₂H₅, etc.); R₂=CH₂, or CO—NH—CH₂CH₂; and Q₁ and Q₂ may be the same or different.

For example, when Q₂═OCH₂CH₂ (there is no Q₁ in this case); Y═—CO₂—N(COCH₂)₂; and X═—NH₂, —SH, or —OH, the resulting reactions and Z groups would be as follows:
Polymer-NH₂+Polymer-O—CH₂—CH₂—CO₂—N(COCH₂)₂→Polymer-NH—CO—CH₂—CH₂—O-Polymer;
Polymer-SH+Polymer-O—CH₂—CH₂—CO₂—N(COCH₂)₂→Polymer-S—COCH₂CH₂—O-Polymer; and
Polymer-OH+Polymer-O—CH₂—CH₂—CO₂—N(COCH₂)₂→Polymer-O—COCH₂CH₂—O-Polymer.

An additional group, represented below as “D”, can be inserted between the polymer and the linking group, if present. One purpose of such a D group is to affect the degradation rate of the crosslinked polymer composition in vivo, for example, to increase the degradation rate, or to decrease the degradation rate. This may be useful in many instances, for example, when drug has been incorporated into the matrix, and it is desired to increase or decrease polymer degradation rate so as to influence a drug delivery profile in the desired direction. An illustration of a crosslinking reaction involving first and second synthetic polymers each having D and Q groups is shown below.
Polymer-D-Q-X+Polymer-D-Q-Y→Polymer-D-Q-Z-Q-D-Polymer

Some useful biodegradable groups “D” include polymers formed from one or more α-hydroxy acids, e.g., lactic acid, glycolic acid, and the cyclization products thereof (e.g., lactide, glycolide), ε-caprolactone, and amino acids. The polymers may be referred to as polylactide, polyglycolide, poly(co-lactide-glycolide); poly-ε-caprolactone, polypeptide (also known as poly amino acid, for example, various di- or tri-peptides) and poly(anhydride)s.

In a general method for preparing the crosslinked polymer compositions used in the context of the present invention, a first synthetic polymer containing multiple nucleophilic groups is mixed with a second synthetic polymer containing multiple electrophilic groups. Formation of a three-dimensional crosslinked network occurs as a result of the reaction between the nucleophilic groups on the first synthetic polymer and the electrophilic groups on the second synthetic polymer.

The concentrations of the first synthetic polymer and the second synthetic polymer used to prepare the compositions of the present invention will vary depending upon a number of factors, including the types and molecular weights of the particular synthetic polymers used and the desired end use application. In general, when using multi-amino PEG as the first synthetic polymer, it is preferably used at a concentration in the range of about 0.5 to about 20 percent by weight of the final composition, while the second synthetic polymer is used at a concentration in the range of about 0.5 to about 20 percent by weight of the final composition. For example, a final composition having a total weight of 1 gram (1000 milligrams) would contain between about 5 to about 200 milligrams of multi-amino PEG, and between about 5 to about 200 milligrams of the second synthetic polymer.

Use of higher concentrations of both first and second synthetic polymers will result in the formation of a more tightly crosslinked network, producing a stiffer, more robust gel. Compositions intended for use in tissue augmentation will generally employ concentrations of first and second synthetic polymer that fall toward the higher end of the preferred concentration range. Compositions intended for use as bioadhesives or in adhesion prevention do not need to be as firm and may therefore contain lower polymer concentrations.

Because polymers containing multiple electrophilic groups will also react with water, the second synthetic polymer is generally stored and used in sterile, dry form to prevent the loss of crosslinking ability due to hydrolysis which typically occurs upon exposure of such electrophilic groups to aqueous media. Processes for preparing synthetic hydrophilic polymers containing multiple electrophylic groups in sterile, dry form are set forth in U.S. Pat. No. 5,643,464. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates. In contrast, polymers containing multiple nucleophilic groups are generally not water-reactive and can therefore be stored in aqueous solution.

In certain embodiments, one or both of the electrophilic- or nucleophilic-terminated polymers described above can be combined with a synthetic or naturally occurring polymer. The presence of the synthetic or naturally occurring polymer may enhance the mechanical and/or adhesive properties of the in situ forming compositions. Naturally occurring polymers, and polymers derived from naturally occurring polymer that may be included in in situ forming materials include naturally occurring proteins, such as collagen, collagen derivatives (such as methylated collagen), fibrinogen, thrombin, albumin, fibrin, and derivatives of and naturally occurring polysaccharides, such as glycosaminoglycans, including deacetylated and desulfated glycosaminoglycan derivatives.

In certain embodiments, a composition comprising naturally-occurring protein and both of the first and second synthetic polymer as described above is used to form the crosslinked matrix according to the present invention. In certain embodiments, a composition comprising collagen and both of the first and second synthetic polymer as described above is used to form the crosslinked matrix according to the present invention. In certain embodiments, a composition comprising methylated collagen and both of the first and second synthetic polymer as described above is used to form the crosslinked matrix according to the present invention. In certain embodiments, a composition comprising fibrinogen and both of the first and second synthetic polymer as described above is used to form the crosslinked matrix according to the present invention. In certain embodiments, a composition comprising thrombin and both of the first and second synthetic polymer as described above is used to form the crosslinked matrix according to the present invention. In certain embodiments, a composition comprising albumin and both of the first and second synthetic polymer as described above is used to form the crosslinked matrix according to the present invention. In certain embodiments, a composition comprising fibrin and both of the first and second synthetic polymer as described above is used to form the crosslinked matrix according to the present invention. In certain embodiments, a composition comprising naturally occurring polysaccharide and both of the first and second synthetic polymer as described above is used to form the crosslinked matrix according to the present invention. In certain embodiments, a composition comprising glycosaminoglycan and both of the first and second synthetic polymer as described above is used to form the crosslinked matrix according to the present invention. In certain embodiments, a composition comprising deacetylated glycosaminoglycan and both of the first and second synthetic polymer as described above is used to form the crosslinked matrix according to the present invention. In certain embodiments, a composition comprising desulfated glycosaminoglycan and both of the first and second synthetic polymer as described above is used to form the crosslinked matrix according to the present invention.

In certain embodiments, a composition comprising naturally-occurring protein and the first synthetic polymer as described above is used to form the crosslinked matrix according to the present invention. In certain embodiments, a composition comprising collagen and the first synthetic polymer as described above is used to form the crosslinked matrix according to the present invention. In certain embodiments, a composition comprising methylated collagen and the first synthetic polymer as described above is used to form the crosslinked matrix according to the present invention. In certain embodiments, a composition comprising fibrinogen and the first synthetic polymer as described above is used to form the crosslinked matrix according to the present invention. In certain embodiments, a composition comprising thrombin and the first synthetic polymer as described above is used to form the crosslinked matrix according to the present invention. In certain embodiments, a composition comprising albumin and the first synthetic polymer as described above is used to form the crosslinked matrix according to the present invention. In certain embodiments, a composition comprising fibrin and the first synthetic polymer as described above is used to form the crosslinked matrix according to the present invention. In certain embodiments, a composition comprising naturally occurring polysaccharide and the first synthetic polymer as described above is used to form the crosslinked matrix according to the present invention. In certain embodiments, a composition comprising glycosaminoglycan and the first synthetic polymer as described above is used to form the crosslinked matrix according to the present invention. In certain embodiments, a composition comprising deacetylated glycosaminoglycan and the first synthetic polymer as described above is used to form the crosslinked matrix according to the present invention. In certain embodiments, a composition comprising desulfated glycosaminoglycan and the first synthetic polymer as described above is used to form the crosslinked matrix according to the present invention.

In certain embodiments, a composition comprising naturally-occurring protein and the second synthetic polymer as described above is used to form the crosslinked matrix according to the present invention. In certain embodiments, a composition comprising collagen and the second synthetic polymer as described above is used to form the crosslinked matrix according to the present invention. In certain embodiments, a composition comprising methylated collagen and the second synthetic polymer as described above is used to form the crosslinked matrix according to the present invention. In certain embodiments, a composition comprising fibrinogen and the second synthetic polymer as described above is used to form the crosslinked matrix according to the present invention. In certain embodiments, a composition comprising thrombin and the second synthetic polymer as described above is used to form the crosslinked matrix according to the present invention. In certain embodiments, a composition comprising albumin and the second synthetic polymer as described above is used to form the crosslinked matrix according to the present invention. In certain embodiments, a composition comprising fibrin and the second synthetic polymer as described above is used to form the crosslinked matrix according to the present invention. In certain embodiments, a composition comprising naturally occurring polysaccharide and the second synthetic polymer as described above is used to form the crosslinked matrix according to the present invention. In certain embodiments, a composition comprising glycosaminoglycan and the second synthetic polymer as described above is used to form the crosslinked matrix according to the present invention. In certain embodiments, a composition comprising deacetylated glycosaminoglycan and the second synthetic polymer as described above is used to form the crosslinked matrix according to the present invention. In certain embodiments, a composition comprising desulfated glycosaminoglycan and the second synthetic polymer as described above is used to form the crosslinked matrix according to the present invention.

The presence of protein or polysaccharide components which contain functional groups that can react with the functional groups on multiple activated synthetic polymers can result in formation of a crosslinked synthetic polymer-naturally occurring polymer matrix upon mixing and/or crosslinking of the synthetic polymer(s). In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix. For example, lysine-rich proteins such as collagen may be especially reactive with electrophilic groups on synthetic polymers.

In certain embodiments, the naturally occurring protein is polymer may be collagen. As used herein, the term “collagen” or “collagen material” refers to all forms of collagen, including those which have been processed or otherwise modified and is intended to encompass collagen of any type, from any source, including, but not limited to, collagen extracted from tissue or produced recombinantly, collagen analogues, collagen derivatives, modified collagens, and denatured collagens, such as gelatin.

In general, collagen from any source may be included in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022 discloses methods of extracting and purifying collagen from the human placenta. U.S. Pat. No. 5,667,839, discloses methods of producing recombinant human collagen in the milk of transgenic animals, including transgenic cows. Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Corporation (Santa Barbara, Calif.) at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Because of its tacky consistency, nonfibrillar collagen may be preferred for use in compositions that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids (e.g., arginine), inorganic salts (e.g., sodium chloride and potassium chloride), and carbohydrates (e.g., various sugars including sucrose).

In certain embodiments, the polymer may be collagen or a collagen derivative, for example methylated collagen. An example of an in situ forming composition uses pentaerythritol poly(ethylene glycol)ether tetra-sulfhydryl] (4-armed thiol PEG), pentaerythritol poly(ethylene glycol)ether tetra-succinimidyl glutarate] (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another aspect, the naturally occurring polymer may be a glycosaminoglycan. Glycosaminoglycans, e.g., hyaluronic acid, contain both anionic and cationic functional groups along each polymeric chain, which can form intramolecular and/or intermolecular ionic crosslinks, and are responsible for the thixotropic (or shear thinning) nature of hyaluronic acid.

In certain aspects, the glycosaminoglycan may be derivatized. For example, glycosaminoglycans can be chemically derivatized by, e.g., deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen. In addition, the introduction of hydrocolloids such as carboxymethylcellulose may promote tissue adhesion and/or swellability.

Administration of the Crosslinked Synthetic Polymer Compositions

The compositions of the present invention having two synthetic polymers may be administered before, during or after crosslinking of the first and second synthetic polymer. Certain uses, which are discussed in greater detail below, such as tissue augmentation, may require the compositions to be crosslinked before administration, whereas other applications, such as tissue adhesion, require the compositions to be administered before crosslinking has reached “equilibrium.” The point at which crosslinking has reached equilibrium is defined herein as the point at which the composition no longer feels tacky or sticky to the touch.

In order to administer the composition prior to crosslinking, the first synthetic polymer and second synthetic polymer may be contained within separate barrels of a dual-compartment syringe. In this case, the two synthetic polymers do not actually mix until the point at which the two polymers are extruded from the tip of the syringe needle into the patient's tissue. This allows the vast majority of the crosslinking reaction to occur in situ, avoiding the problem of needle blockage which commonly occurs if the two synthetic polymers are mixed too early and crosslinking between the two components is already too advanced prior to delivery from the syringe needle. The use of a dual-compartment syringe, as described above, allows for the use of smaller diameter needles, which is advantageous when performing soft tissue augmentation in delicate facial tissue, such as that surrounding the eyes.

Alternatively, the first synthetic polymer and second synthetic polymer may be mixed according to the methods described above prior to delivery to the tissue site, then injected to the desired tissue site immediately (preferably, within about 60 seconds) following mixing.

In another embodiment of the invention, the first synthetic polymer and second synthetic polymer are mixed, then extruded and allowed to crosslink into a sheet or other solid form. The crosslinked solid is then dehydrated to remove substantially all unbound water. The resulting dried solid may be ground or comminuted into particulates, then suspended in a nonaqueous fluid carrier, including, without limitation, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid. The suspension of particulates can be injected through a small-gauge needle to a tissue site. Once inside the tissue, the crosslinked polymer particulates will rehydrate and swell in size at least five-fold.

Hydrophilic Polymer+Plurality of Crosslinkable Components

As mentioned above, the first and/or second synthetic polymers may be combined with a hydrophilic polymer, e.g., collagen or methylated collagen, to form a composition useful in the present invention. In one general embodiment, the compositions useful in the present invention include a hydrophilic polymer in combination with two or more crosslinkable components. This embodiment is described in further detail in this section.

The Hydrophilic Polymer Component:

The hydrophilic polymer component may be a synthetic or naturally occurring hydrophilic polymer. Naturally occurring hydrophilic polymers include, but are not limited to: proteins such as collagen and derivatives thereof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. See, e.g., U.S. Pat. No. 5,428,022, to Palefsky et al., which discloses methods of extracting and purifying collagen from the human placenta. See also U.S. Pat. No. 5,667,839, to Berg, which discloses methods of producing recombinant human collagen in the milk of transgenic animals, including transgenic cows. Unless otherwise specified, the term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from McGhan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in U.S. Pat. No. 5,614,587 to Rhee et al.

Collagens for use in the crosslinkable compositions of the present invention may start out in fibrillar form, then be rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred. However, as disclosed in U.S. Pat. No. 5,614,587, fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide)copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono-and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The Crosslinkable Components:

The compositions of the invention also comprise a plurality of crosslinkable components. Each of the crosslinkable components participates in a reaction that results in a crosslinked matrix. Prior to completion of the crosslinking reaction, the crosslinkable components provide the necessary adhesive qualities that enable the methods of the invention.

The crosslinkable components are selected so that crosslinking gives rise to a biocompatible, nonimmunogenic matrix useful in a variety of contexts including adhesion prevention, biologically active agent delivery, tissue augmentation, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A, or nucleophilic and capable of reaction with the electrophilic groups of component B may also be present. Thus, the total number of functional groups present on components A, B and C, when present, in combination is≧5; that is, the total functional groups given by m+n+p must be≧5, where p is the number of functional groups on component C and, as indicated, is≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

The m nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae
R¹(-[Q¹]_{q}-X)ₘ (component A), (I)
R²(-[Q²]ᵣ-Y)ₙ (component B), and (II)
R³(-[Q³]ₛ-Fn)ₚ (optional component C), (I)
wherein:

R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;

X represents one of the m nucleophilic groups of component A, and the various X moieties on A may be the same or different;

Y represents one of the n electrophilic groups of component B, and the various Y moieties on A may be the same or different;

Fn represents a functional group on optional component C;

Q¹, Q² and Q³ are linking groups;

m≧2, n≧2, m+n is≧4, q, and r are independently zero or 1, and when optional component C is present, p≧1, and s is independently zero or 1.

Reactive Groups:

X may be virtually any nucleophilic group, so long as reaction can occur with the electrophilic group Y. Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities—R₆MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra in Section E.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—CO₂-C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imidothioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups are particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE 7

| REPRESENTATIVE | | |
|---|---|---|
| NUCLEOPHILIC | | |
| COMPONENT | REPRESENTATIVE | |
| (A, optional | ELECTROPHILIC | |
| component C element | COMPONENT | |
| FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyl carbonate terminus) | |
| R¹—SH | R²—O—(CO)—O—N(COCH₂) | R¹—S—(CO)—O—R² |
| R¹—OH | R²—O—(CO)—O—N(COCH₂) | R¹—O—(CO)—R² |
| R¹—NH₂ | R²—O(CO)—CH═CH₂ | R¹—NH—CH₂CH₂—(CO)—O—R² |
| | (acrylate terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂—N(COCH₂) | R¹—NH—(CO)—(CH₂)₃—(CO)—OR² |
| | (succinimidyl glutarate terminus) | |
| R¹—SH | R²—O(CO)—(CH₂)₃—CO₂—N(COCH₂) | R¹—S—(CO)—(CH₂)₃—(CO)—OR² |
| R¹—OH | R²—O(CO)—(CH₂)₃—CO₂—N(COCH₂) | R¹—O—(CO)—(CH₂)₃—(CO)—OR² |
| R¹—NH₂ | R²—O—CH₂—CO₂—N(COCH₂) | R¹—NH—(CO)—CH₂—OR² |
| | (succinimidyl acetate terminus) | |
| R¹—SH | R²—O—CH₂—CO₂—N(COCH₂) | R¹—S—(CO)—CH₂—OR² |
| R¹—OH | R²—O—CH₂—CO₂—N(COCH₂) | R¹—O—(CO)—CH₂—OR² |
| R¹—NH₂ | R²—O—NH(CO)—(CH₂)₂—CO₂—N(COCH₂) | R¹—NH—(CO)—(CH₂)₂—(CO)—NH—OR² |
| | (succinimidyl succinamide terminus) | |
| R¹—SH | R²—O—NH(CO)—(CH₂)₂—CO₂—N(COCH₂) | R¹—S—(CO)—(CH₂)₂—(CO)—NH—OR² |
| R¹—OH | R²—O—NH(CO)—(CH₂)₂—CO₂—N(COCH₂) | R¹—O—(CO)—(CH₂)₂—(CO)—NH—OR² |
| R¹—NH₂ | R²—O—(CH₂)₂—CHO | R¹—NH—(CO)—(CH₂)₂—OR² |
| | (propionaldehyde terminus) | |
| R¹—NH₂ | 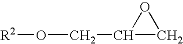 (glycidyl ether terminus) | R¹—NH—CH₂—CH(OH)—CH₂—OR²and R¹—N[CH₂—CH(OH)—CH₂—OR²]₂ |
| R¹NH₂ | R²—O—(CH₂)₂—N═C═O | R¹—NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹SH | R²—SO₂—CH═CH₂ | R¹—S—CH₂CH₂—SO₂—R² |

Linking Groups:

The functional groups X and Y and FN on optional component C may be directly attached to the compound core (R¹, R₂ or R₃ on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, International Patent Publication No. WO 97/22371. Linking groups are useful to avoid steric hindrance problems that are sometimes associated with the formation of direct linkages between molecules. Linking groups may additionally be used to link several multifinctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia: ester linkages; anhydride linkages, such as obtained by incorporation of glutarate and succinate; ortho ester linkages; ortho carbonate linkages such as trimethylene carbonate; amide linkages; phosphoester linkages; a-hydroxy acid linkages, such as may be obtained by incorporation of lactic acid and glycolic acid; lactone-based linkages, such as may be obtained by incorporation of caprolactone, valerolactone, y-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in the following Table 8:

TABLE 8

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ-Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ-Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ-Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ-Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ-Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ-Z |
| —(CO)—O—(CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ-Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ-Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷-Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH-Z |

In the above Table, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows: If higher molecular weight components are to be used, they preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Component Core:

The “core” of each crosslinkable component is comprised of the molecular structure to which the nucleophilic or electrophilic groups are bound. Using the formulae (I) R¹-[Q¹]_{q}-X)ₘ, for component A, (II) R²(-[Q²]ᵣ-Y)ₙ for component B, and (III)

R³(-[Q³]ₛ-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to 2 heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

Hydrophilic Crosslinkable Components:

In certain embodiments, the crosslinkable component(s) is (are) hydrophilic polymers. The term “hydrophilic polymer” as used herein refers to a synthetic polymer having an average molecular weight and composition effective to render the polymer “hydrophilic” as defined above. As discussed above, synthetic crosslinkable hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic crosslinkable hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic crosslinkable hydrophilic polymers can be used in the present compositions, as indicated above, preferred synthetic crosslinkable hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and do not typically interfere with the enzymatic activities and/or conformations of peptides. A particularly preferred synthetic crosslinkable hydrophilic polymer for certain applications is a polyethylene glycol (PEG) having a molecular weight within the range of about 100 to about 100,000 mol. wt., although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 mol. wt., more preferably within the range of about 7,500 to about 20,000 mol. wt. Most preferably, the polyethylene glycol has a molecular weight of approximately 10,000 mol. wt.

Naturally occurring crosslinkable hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6:315), peptide chemistry (see Mutter et al., The Peptides, Academic: New York, N.Y. 2:285-332; and Zalipsky et al., Int. J. Peptide Protein Res. (1987) 30:740), and the synthesis of polymeric drugs (see Zalipsky et al., Eur. Polym. J. (1983) 19:1177; and Ouchi et al., J. Macromol. Sci. Chem. (1987) A24:1011).

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Alabama (1997-1998).

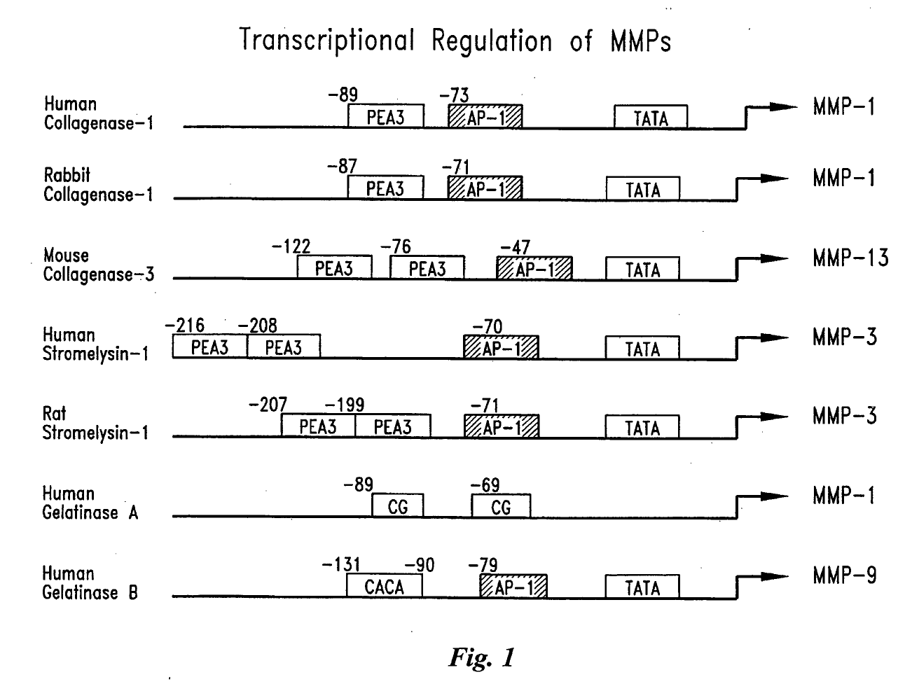

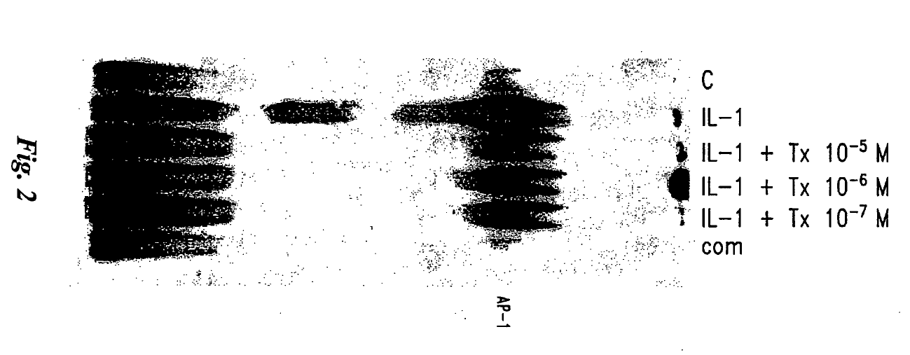

Structures for some specific, tetrafunctionally activated forms of PEG are shown in FIGS. 1 to 10 of U.S. Pat. No. 5,874,500, as are generalized reaction products obtained by reacting the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

Hydrophobic Polymers:

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

Low Molecular Weight Components:

As indicated above, the molecular core of one or more of the crosslinkable components can also be a low molecular weight compound, i.e., a C₂-C₁₄ hydrocarbyl group containing zero to 2 heteroatoms selected from N, O, S and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di- and poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The aforementioned compounds are commercially available from Pierce (Rockford, Ill.). Such di- and poly-electrophiles can also be synthesized from di- and polyacids, for example by reaction with an appropriate molar amount of N-hydroxysuccinimide in the presence of DCC. Polyols such as trimethylolpropane and di(trimethylol propane) can be converted to carboxylic acid form using various known techniques, then further derivatized by reaction with NHS in the presence of DCC to produce trifunctionally and tetrafunctionally activated polymers.

Delivery Systems:

Suitable delivery systems for the homogeneous dry powder composition (containing at least two crosslinkable polymers) and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution.

In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. No. 6,454,786 to Holm et al.; U.S. Pat. No. 6,461,325 to Delmotte et al.; U.S. Pat. No. 5,585,007 to Antanavich et al.; U.S. Pat. No. 5,116,315 to Capozzi et al.; and U.S. Pat. No. 4,631,055 to Redl et al.

Storage and Handling:

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1-dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

Collagen+Fibrinogen and/or Thrombin (e.g., Costasis)

In yet another aspect, the polymer composition may include collagen in combination with fibrinogen and/or thrombin. (See, e.g., U.S. Pat. Nos. 5,290,552; 6,096,309; and 5,997,811). For example, an aqueous composition may include a fibrinogen and FXIII, particularly plasma, collagen in an amount sufficient to thicken the composition, thrombin in an amount sufficient to catalyze polymerization of fibrinogen present in the composition, and Ca²⁺ and, optionally, an antifibrinolytic agent in amount sufficient to retard degradation of the resulting adhesive clot. The composition may be formulated as a two-part composition that may be mixed together just prior to use, in which fibrinogen/FXIII and collagen constitute the first component, and thrombin together with an antifibrinolytic agent, and Ca²⁺ constitute the second component.

Plasma, which provides a source of fibrinogen, may be obtained from the patient for which the composition is to be delivered. The plasma can be used “as is” after standard preparation which includes centrifuging out cellular components of blood. Alternatively, the plasma can be further processed to concentrate the fibrinogen to prepare a plasma cryoprecipitate. The plasma cryoprecipitate can be prepared by freezing the plasma for at least about an hour at about −20° C., and then storing the frozen plasma overnight at about 4° C. to slowly thaw. The thawed plasma is centrifuged and the plasma cryoprecipitate is harvested by removing approximately four-fifths of the plasma to provide a cryoprecipitate comprising the remaining one-fifth of the plasma. Other fibrinogen/FXIII preparations may be used, such as cryoprecipitate, patient autologous fibrin sealant, fibrinogen analogs or other single donor or commercial fibrin sealant materials. Approximately 0.5 ml to about 1.0 ml of either the plasma or the plasma-cryoprecipitate provides about 1 to 2 ml of adhesive composition which is sufficient for use in middle ear surgery. Other plasma proteins (e.g., albumin, plasminogen, von Willebrands factor, Factor VIII, etc.) may or may not be present in the fibrinogen/FXII separation due to wide variations in the formulations and methods to derive them.

Collagen, preferably hypoallergenic collagen, is present in the composition in an amount sufficient to thicken the composition and augment the cohesive properties of the preparation. The collagen may be atelopeptide collagen or telopeptide collagen, e.g., native collagen. In addition to thickening the composition, the collagen augments the fibrin by acting as a macromolecular lattice work or scaffold to which the fibrin network adsorbs. This gives more strength and durability to the resulting glue clot with a relatively low concentration of fibrinogen in comparison to the various concentrated autogenous fibrinogen glue formulations (i.e., AFGs).

The form of collagen which is employed may be described as at least “near native” in its structural characteristics. It may be further characterized as resulting in insoluble fibers at a pH above 5; unless crosslinked or as part of a complex composition, e.g., bone, it will generally consist of a minor amount by weight of fibers with diameters greater than 50 nm, usually from about 1 to 25 volume % and there will be substantially little, if any, change in the helical structure of the fibrils. In addition, the collagen composition must be able to enhance gelation in the surgical adhesion composition.

A number of commercially available collagen preparations may be used. ZYDERM Collagen Implant (ZCI) has a fibrillar diameter distribution consisting of 5 to 10 nm diameter fibers at 90% volume content and the remaining 10% with greater than about 50 nm diameter fibers. ZCI is available as a fibrillar slurry and solution in phosphate buffered isotonic saline, pH 7.2, and is injectable with fine gauge needles. As distinct from ZCI, cross-linked collagen available as ZYPLAST may be employed. ZYPLAST is essentially an exogenously crosslinked (glutaraldehyde) version of ZCI. The material has a somewhat higher content of greater than about 50 nm diameter fibrils and remains insoluble over a wide pH range. Crosslinking has the effect of mimicking in vivo endogenous crosslinking found in many tissues.

Thrombin acts as a catalyst for fibrinogen to provide fibrin, an insoluble polymer and is present in the composition in an amount sufficient to catalyze polymerization of fibrinogen present in the patient plasma. Thrombin also activates FXIII, a plasma protein that catalyzes covalent crosslinks in fibrin, rendering the resultant clot insoluble. Usually the thrombin is present in the adhesive composition in concentration of from about 0.01 to about 1000 or greater NIH units (NIHu) of activity, usually about i to about 500 NIHu, most usually about 200 to about 500 NIHu. The thrombin can be from a variety of host animal sources, conveniently bovine. Thrombin is commercially available from a variety of sources including Parke-Davis, usually lyophilized with buffer salts and stabilizers in vials which provide thrombin activity ranging from about 1000 NIHu to 10,000 NIHu. The thrombin is usually prepared by reconstituting the powder by the addition of either sterile distilled water or isotonic saline. Alternately, thrombin analogs or reptile-sourced coagulants may be used.

The composition may additionally comprise an effective amount of an antifibrinolytic agent to enhance the integrity of the glue clot as the healing processes occur. A number of antifibrinolytic agents are well known and include aprotinin, C1-esterase inhibitor and ε-amino-n-caproic acid (EACA). ε-amino-n-caproic acid, the only antifibrinolytic agent approved by the FDA, is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration. That solution is desirably used to reconstitute lyophilized thrombin to the desired thrombin concentration.

Other examples of in situ forming materials based on the crosslinking of proteins are described, e.g., in U.S. Pat. Nos. RE38158; 4,839,345; 5,514,379, 5,583,114; 6,458,147; 6,371,975; 5,290,552; 6,096,309; U.S. Pat. Application Publication Nos. 2002/0161399; 2001/0018598 and PCT Publication Nos. WO 03/090683; WO 01/45761; WO 99/66964 and WO 96/03159).

Self-Reactive Compounds

In certain embodiments, the drug combination or individual component(s) thereof is released from a crosslinked matrix formed, at least in part, from a self-reactive compound. As used herein, a self-reactive compound comprises a core substituted with a minimum of three reactive groups. The reactive groups may be directed attached to the core of the compound, or the reactive groups may be indirectly attached to the compound's core, e.g., the reactive groups are joined to the core through one or more linking groups.

Each of the three reactive groups that are necessarily present in a self-reactive compound can undergo a bond-forming reaction with at least one of the remaining two reactive groups. For clarity it is mentioned that when these compounds react to form a crosslinked matrix, it will most often happen that reactive groups on one compound will reactive with reactive groups on another compound. That is, the term “self-reactive” is not intended to mean that each self-reactive compound necessarily reacts with itself, but rather that when a plurality of identical self-reactive compounds are in combination and undergo a crosslinking reaction, then these compounds will react with one another to form the matrix. The compounds are “self-reactive” in the sense that they can react with other compounds having the identical chemical structure as themselves.

The self-reactive compound comprises at least four components: a core and three reactive groups. In one embodiment, the self-reactive compound can be characterized by the formula (I), where R is the core, the reactive groups are represented by X¹, X² and X³, and a linker (L) is optionally present between the core and a functional group.

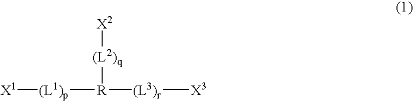

The core R is a polyvalent moiety having attachment to at least three groups (i.e., it is at least trivalent) and may be, or may contain, for example, a hydrophilic polymer, a hydrophobic polymer, an amphiphilic polymer, a C₂₋₁₄ hydrocarbyl, or a C₂₋₁₄ hydrocarbyl which is heteroatom-containing. The linking groups L¹, L², and L³ may be the same or different. The designators p, q and r are either 0 (when no linker is present) or 1 (when a linker is present). The reactive groups X¹, X² and X³ may be the same or different. Each of these reactive groups reacts with at least one other reactive group to form a three-dimensional matrix. Therefore X¹ can react with X² and/or X³, X² can react with X¹ and/or X³, X³ can react with X¹ and/or X² and so forth. A trivalent core will be directly or indirectly bonded to three functional groups, a tetravalent core will be directly or indirectly bonded to four functional groups, etc.

Each side chain typically has one reactive group. However, the invention also encompasses self-reactive compounds where the side chains contain more than one reactive group. Thus, in another embodiment of the invention, the self-reactive compound has the formula (II):
[X′-(L⁴)ₐ-Y′-(L⁵)_{b}]_{c}-R′
where: a and b are integers from 0-1; c is an integer from 3-12; R′ is selected from hydrophilic polymers, hydrophobic polymers, amphiphilic polymers, C₂₋₁₄ hydrocarbyls, and heteroatom-containing C₂₋₁₄ hydrocarbyls; X′ and Y′ are reactive groups and can be the same or different; and L⁴ and L⁵ are linking groups. Each reactive group inter-reacts with the other reactive group to form a three-dimensional matrix. The compound is essentially non-reactive in an initial environment but is rendered reactive upon exposure to a modification in the initial environment that provides a modified environment such that a plurality of the self-reactive compounds inter-react in the modified environment to form a three-dimensional matrix. In one preferred embodiment, R is a hydrophilic polymer. In another preferred embodiment, X′ is a nucleophilic group and Y′ is an electrophilic group.

The following self-reactive compound is one example of a compound of formula (II): 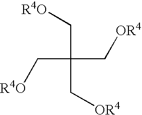
where R⁴ has the formula:

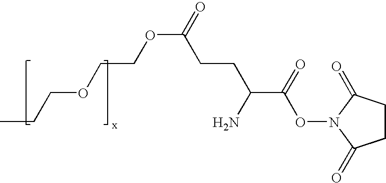

Thus, in formula (II), a and b are 1; c is 4; the core R′ is the hydrophilic polymer, tetrafunctionally activated polyethylene glycol, (C(CH₂—O—)₄; X′ is the electrophilic reactive group, succinimidyl; Y′ is the nucleophilic reactive group —CH—NH₂; L⁴ is —C(O)—O—; and L⁵ is —(CH₂—CH₂—O—CH₂)ₓ—CH₂—O—C(O)—(CH₂)₂—.

The self-reactive compounds of the invention are readily synthesized by techniques that are well known in the art. An exemplary synthesis is set forth below:

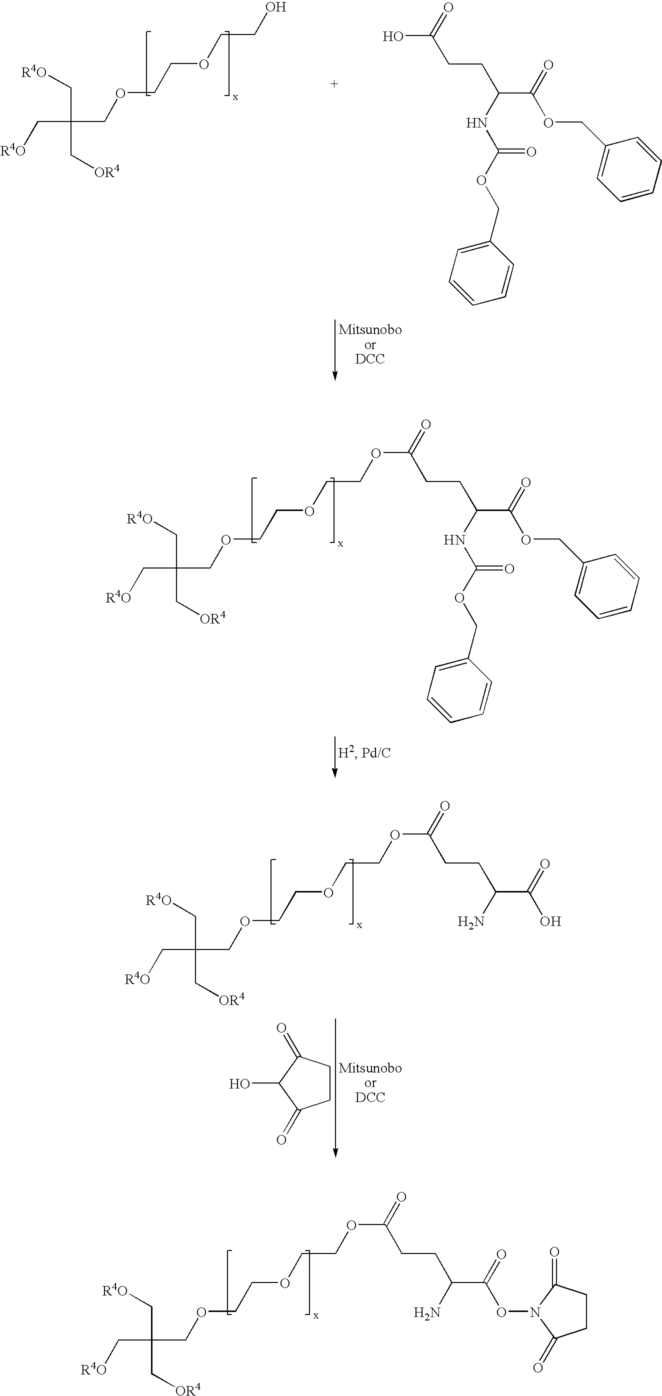

The reactive groups are selected so that the compound is essentially non-reactive in an initial environment. Upon exposure to a specific modification in the initial environment, providing a modified environment, the compound is rendered reactive and a plurality of self-reactive compounds are then able to inter-react in the modified environment to form a three-dimensional matrix. Examples of modification in the initial environment are detailed below, but include the addition of an aqueous medium, a change in pH, exposure to ultraviolet radiation, a change in temperature, or contact with a redox initiator.

The core and reactive groups can also be selected so as to provide a compound that has one of more of the following features: are biocompatible, are non-immunogenic, and do not leave any toxic, inflammatory or immunogenic reaction products at the site of administration. Similarly, the core and reactive groups can also be selected so as to provide a resulting matrix that has one or more of these features.

In one embodiment of the invention, substantially immediately or immediately upon exposure to the modified environment, the self-reactive compounds inter-react form a three-dimensional matrix. The term “substantially immediately” is intended to mean within less than five minutes, preferably within less than two minutes, and the term “immediately” is intended to mean within less than one minute, preferably within less than 30 seconds.

In one embodiment, the self-reactive compound and resulting matrix are not subject to enzymatic cleavage by matrix metalloproteinases such as collagenase, and are therefore not readily degradable in vivo. Further, the self-reactive compound may be readily tailored, in terms of the selection and quantity of each component, to enhance certain properties, e.g., compression strength, swellability, tack, hydrophilicity, optical clarity, and the like.

In one preferred embodiment, R is a hydrophilic polymer. In another preferred embodiment, X is a nucleophilic group, Y is an electrophilic group and Z is either an electrophilic or a nucleophilic group. Additional embodiments are detailed below.

A higher degree of inter-reaction, e.g., crosslinking, may be useful when a less swellable matrix is desired or increased compressive strength is desired. In those embodiments, it may be desirable to have n be an integer from 2-12. In addition, when a plurality of self-reactive compounds are utilized, the compounds may be the same or different.

Reactive Groups

Prior to use, the self-reactive compound is stored in an initial environment that insures that the compound remain essentially non-reactive until use. Upon modification of this environment, the compound is rendered reactive and a plurality of compounds will then inter-react to form the desired matrix. The initial environment, as well as the modified environment, is thus determined by the nature of the reactive groups involved.

The number of reactive groups can be the same or different. However, in one embodiment of the invention, the number of reactive groups are approximately equal. As used in this context, the term “approximately” refers to a 2:1 to 1:2 ratio of moles of one reactive group to moles of a different reactive groups. A 1:1:1 molar ratio of reactive groups is generally preferred.

In general, the concentration of the self-reactive compounds in the modified environment, when liquid in nature, will be in the range of about 1 to 50 wt %, generally about 2 to 40 wt %. The preferred concentration of the compound in the liquid will depend on a number of factors, including the type of compound (i.e., type of molecular core and reactive groups), its molecular weight, and the end use of the resulting three-dimensional matrix. For example, use of higher concentrations of the compounds, or using highly functionalized compounds, will result in the formation of a more tightly crosslinked network, producing a stiffer, more robust gel. As such, compositions intended for use in tissue augmentation will generally employ concentrations of self-reactive compounds that fall toward the higher end of the preferred concentration range. Compositions intended for use as bioadhesives or in adhesion prevention do not need to be as firm and may therefore contain lower concentrations of the self-reactive compounds.

Electrophilic and Nucleophilic Reactive Groups:

In one embodiment of the invention, the reactive groups are electrophilic and nucleophilic groups, which undergo a nucleophilic substitution reaction, a nucleophilic addition reaction, or both. The term “electrophilic” refers to a reactive group that is susceptible to nucleophilic attack, i.e., susceptible to reaction with an incoming nucleophilic group. Electrophilic groups herein are positively charged or electron-deficient, typically electron-deficient. The term “nucleophilic” refers to a reactive group that is electron rich, has an unshared pair of electrons acting as a reactive site, and reacts with a positively charged or electron-deficient site. For such reactive groups, the modification in the initial environment comprises the addition of an aqueous medium and/or a change in pH.

In one embodiment of the invention, X1 (also referred to herein as X) can be a nucleophilic group and X2 (also referred to herein as Y) can be an electrophilic group or vice versa, and X3 (also referred to herein as Z) can be either an electrophilic or a nucleophilic group.

X may be virtually any nucleophilic group, so long as reaction can occur with the electrophilic group Y and also with Z, when Z is electrophilic (Z_{EL}). Analogously, Y may be virtually any electrophilic group, so long as reaction can take place with X and also with Z when Z is nucleophilic (Z_{NU}). The only limitation is a practical one, in that reaction between X and Y, and X and Z_{EL}, or Y and Z_{NU} should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. In one embodiment, the reactions between X and Y, and between either X and Z_{EL} or Y and Z_{NU}, are complete in under 60 minutes, preferably under 30 minutes. Most preferably, the reaction occurs in about 5 to 15 minutes or less.

Examples of nucleophilic groups suitable as X or Fn_{NU} include, but are not limited to: —NH₂, —NHR¹, —N(R¹)₂, —SH, —OH, —COOH, —C₆H₄—OH, —H, —PH₂, —PHR¹, —P(R¹)₂, —NH—NH₂, —CO—NH—NH₂, —C₅H₄N, etc. wherein R¹ is a hydrocarbyl group and each R1 may be the same or different. R¹ is typically alkyl or monocyclic aryl, preferably alkyl, and most preferably lower alkyl. Organometallic moieties are also useful nucleophilic groups for the purposes of the invention, particularly those that act as carbanion donors. Examples of organometallic moieties include: Grignard functionalities —R²MgHal wherein R² is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophilic group. For example, when there are nucleophilic sulfhydryl and hydroxyl groups in the self-reactive compound, the compound must be admixed with an aqueous base in order to remove a proton and provide an —S⁻ or —O⁻ species to enable reaction with the electrophilic group. Unless it is desirable for the base to participate in the reaction, a non-nucleophilic base is preferred. In some embodiments, the base may be present as a component of a buffer solution. Suitable bases and corresponding crosslinking reactions are described herein.

The selection of electrophilic groups provided on the self-reactive compound, must be made so that reaction is possible with the specific nucleophilic groups. Thus, when the X reactive groups are amino groups, the Y and any Z_{EL} groups are selected so as to react with amino groups. Analogously, when the X reactive groups are sulfhydryl moieties, the corresponding electrophilic groups are sulfhydryl-reactive groups, and the like. In general, examples of electrophilic groups suitable as Y or Z_{EL} include, but are not limited to, —CO—Cl, —(CO)—O—(CO)—R (where R is an alkyl group), —CH═CH—CH═O and —CH═CH—C(CH₃)═O, halo, —N═C═O, —N═C═S, —SO₂CH═CH₂, —O(CO)—C═CH₂, —O(CO)—C(CH₃)═CH₂, —S—S—(C₅H₄N), —O(CO)—C(CH₂CH₃)═CH₂, —CH═CH—C═NH, —COOH, —(CO)O—N(COCH₂)₂, —CHO, —(CO)O—N(COCH₂)₂—S(O)₂OH, and —N(COCH)₂.

When X is amino (generally although not necessarily primary amino), the electrophilic groups present on Y and Z_{EL} are amine-reactive groups. Exemplary amine-reactive groups include, by way of example and not limitation, the following groups, or radicals thereof: (1) carboxylic acid esters, including cyclic esters and “activated” esters; (2) acid chloride groups (—CO—Cl); (3) anhydrides (—(CO)—O—(CO)—R, where R is an alkyl group); (4) ketones and aldehydes, including α,β-unsaturated aldehydes and ketones such as —CH═CH—CH═O and —CH═CH—C(CH₃)═O; (5) halo groups; (6) isocyanate group (—N═C═O); (7) thioisocyanato group (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—O(CO)—C═CH₂), methacrylate (—O(CO)—C(CH₃)═CH₂), ethyl acrylate (—O(CO)—C(CH₂CH₃)═CH₂), and ethyleneimino (—CH═CH—C═NH).

In one embodiment the amine-reactive groups contain an electrophilically reactive carbonyl group susceptible to nucleophilic attack by a primary or secondary amine, for example the carboxylic acid esters and aldehydes noted above, as well as carboxyl groups (—COOH).

Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Accordingly, in one embodiment, the amine-reactive groups are selected from succinimidyl ester (—O(CO)—N(COCH₂)₂), sulfosuccinimidyl ester (—O(CO)—N(COCH₂)₂—S(O)₂OH), maleimido (—N(COCH)₂), epoxy, isocyanato, thioisocyanato, and ethenesulfonyl.

Analogously, when X is sulfhydryl, the electrophilic groups present on Y and Z_{EL} are groups that react with a sulfhydryl moiety. Such reactive groups include those that form thioester linkages upon reaction with a sulfhydryl group, such as those described in WO 00/62827 to Wallace et al. As explained in detail therein, sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imidothioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophilic group such as an epoxide group, an aziridine group, an acyl halide, an anhydride, and so forth.

When X is an organometallic nucleophilic group such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophilic or as electrophilic groups, depending on the selected reaction partner and/or the reaction conditions. For example, a carboxylic acid group can act as a nucleophilic group in the presence of a fairly strong base, but generally acts as an electrophilic group allowing nucleophilic attack at the carbonyl carbon and concomitant replacement of the hydroxyl group with the incoming nucleophilic group.

These, as well as other embodiments are illustrated below, where the covalent linkages in the matrix that result upon covalent binding of specific nucleophilic reactive groups to specific electrophilic reactive groups on the self-reactive compound include, solely by way of example, the following Table 9:

TABLE 9

| Representative | | |
|---|---|---|
| Nucleophilic | Representative Electrophilic Group | |
| Group (X, Z_{NU}) | (Y, Z_{EL}) | Resulting Linkage |
| —NH₂ | —O—(CO)—O—N(COCH₂)₂ | —NH—(CO)—O— |
| | succinimidyl carbonate terminus | |
| —SH | —O—(CO)—O—N(COCH₂)₂ | —S—(CO)—O— |
| —OH | —O—(CO)—O—N(COCH₂)₂ | —O—(CO)— |
| —NH₂ | —O(CO)—CH═CH₂ | —NH—CH₂CH₂—(CO)—O— |
| | acrylate terminus | |
| —SH | —O—(CO)—CH═CH₂ | —S—CH₂CH₂—(CO)—O— |
| —OH | —O—(CO)—CH═CH₂ | —O—CH₂CH₂—(CO)—O— |
| —NH₂ | —O(CO)—(CH₂)₃—CO₂—N(COCH₂)₂ | —NH—(CO)—(CH₂)₃—(CO)—O— |
| | succinimidyl glutarate terminus | |
| —SH | —O(CO)—(CH₂)₃—CO₂—N(COCH₂)₂ | —S—(CO)—(CH₂)₃—(CO)—O— |
| —OH | —O(CO)—(CH₂)₃—CO₂—N(COCH₂)₂ | —O—(CO)—(CH₂)₃—(CO)—O— |
| —NH₂ | —O—CH₂—CO₂—N(COCH₂)₂ | —NH—(CO)—CH₂—O— |
| | succinimidyl acetate terminus | |
| —SH | —O—CH₂—CO₂—N(COCH₂)₂ | —S—(CO)—CH₂—O— |
| —OH | —O—CH₂—CO₂—N(COCH₂)₂ | —O—(CO)—CH₂—O— |
| —NH₂ | —O—NH(CO)—(CH₂)₂—CO₂—N(COCH₂)₂ | —NH—(CO)—(CH₂)₂—(CO)—NH—O— |
| | succinimidyl succinamide terminus | |
| —SH | —O—NH(CO)—(CH₂)₂—CO₂—N(COCH₂)₂ | —S—(CO)—(CH₂)₂—(CO)—NH—O— |
| —OH | —O—NH(CO)—(CH₂)₂—CO₂—N(COCH₂)₂ | —O—(CO)—(CH₂)₂—(CO)—NH—O— |
| —NH₂ | —O—(CH₂)₂—CHO | —NH—(CO)—(CH₂)₂—O— |
| | propionaldehyde terminus | |
| —NH₂ | 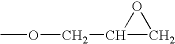 glycidyl ether terminus | —NH—CH₂—CH(OH)—CH₂—O—and —N[CH₂—CH(OH)—CH₂—O—]₂ |
| —NH₂ | —O—(CH₂)₂—N═C═O | —NH—(CO)—NH—CH₂—O— |
| | (isocyanate terminus) | |
| —NH₂ | —SO₂—CH═CH₂ | —NH—CH₂CH₂—SO₂— |
| —SH | —SO₂—CH═CH₂ | —S—CH₂CH₂—SO₂— |

For self-reactive compounds containing electrophilic and nucleophilic reactive groups, the initial environment typically can be dry and sterile. Since electrophilic groups react with water, storage in sterile, dry form will prevent hydrolysis. The dry synthetic polymer may be compression molded into a thin sheet or membrane, which can then be sterilized using gamma or e-beam irradiation. The resulting dry membrane or sheet can be cut to the desired size or chopped into smaller size particulates. The modification of a dry initial environment will typically comprise the addition of an aqueous medium.

In one embodiment, the initial environment can be an aqueous medium such as in a low pH buffer, i.e., having a pH less than about 6.0, in which both electrophilic and nucleophilic groups are non-reactive. Suitable liquid media for storage of such compounds include aqueous buffer solutions such as monobasic sodium phosphate/dibasic sodium phosphate, sodium carbonate/sodium bicarbonate, glutamate or acetate, at a concentration of 0.5 to 300 mM. Modification of an initial low pH aqueous environment will typically comprise increasing the pH to at least pH 7.0, more preferably increasing the pH to at least pH 9.5.

In another embodiment the modification of a dry initial environment comprises dissolving the self-reactive compound in a first buffer solution having a pH within the range of about 1.0 to 5.5 to form a homogeneous solution, and (ii) adding a second buffer solution having a pH within the range of about 6.0 to 11.0 to the homogeneous solution. The buffer solutions are aqueous and can be any pharmaceutically acceptable basic or acid composition. The term “buffer” is used in a general sense to refer to an acidic or basic aqueous solution, where the solution may or may not be functioning to provide a buffering effect (i.e., resistance to change in pH upon addition of acid or base) in the compositions of the present invention. For example, the self-reactive compound can be in the form of a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution. For example, 0.375 grams of the dry powder can be combined with 0.75 grams of the acid buffer to provide, after mixing, a homogeneous solution, where this solution is combined with 1.1 grams of the basic buffer to provide a reactive mixture that substantially immediately forms a three-dimensional matrix.

Acidic buffer solutions having a pH within the range of about 1.0 to 5.5, include by way of illustration and not limitation, solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In a preferred embodiment, the acidic buffer solution, is a solution of citric acid, hydrochloric acid, phosphoric acid, sulfuric acid, and combinations thereof. Regardless of the precise acidifying agent, the acidic buffer preferably has a pH such that it retards the reactivity of the nucleophilic groups on the core. For example, a pH of 2.1 is generally sufficient to retard the nucleophilicity of thiol groups. A lower pH is typically preferred when the core contains amine groups as the nucleophilic groups. In general, the acidic buffer is an acidic solution that, when contacted with nucleophilic groups, renders those nucleophilic groups relatively non-nucleophilic.

An exemplary acidic buffer is a solution of hydrochloric acid, having a concentration of about 6.3 mM and a pH in the range of 2.1 to 2.3. This buffer may be prepared by combining concentrated hydrochloric acid with water, i.e., by diluting concentrated hydrochloric acid with water. Similarly, this buffer A may also be conveniently prepared by diluting 1.23 grams of concentrated hydrochloric acid to a volume of 2 liters, or diluting 1.84 grams of concentrated hydrochloric acid to a volume to 3 liters, or diluting 2.45 grams of concentrated hydrochloric acid to a volume of 4 liters, or diluting 3.07 grams concentrated hydrochloric acid to a volume of 5 liters, or diluting 3.68 grams of concentrated hydrochloric acid to a volume to 6 liters. For safety reasons, the concentrated acid is preferably added to water.

Basic buffer solutions having a pH within the range of about 6.0 to 11.0, include by way of illustration and not limitation, solutions of: glutamate, acetate, carbonate and carbonate salts (e.g., sodium carbonate, sodium carbonate monohydrate and sodium bicarbonate), borate, phosphate and phosphate salts (e.g., monobasic sodium phosphate monohydrate and dibasic sodium phosphate), and combinations thereof. In a preferred embodiment, the basic buffer solution is a solution of carbonate salts, phosphate salts, and combinations thereof.

In general, the basic buffer is an aqueous solution that neutralizes the effect of the acidic buffer, when it is added to the homogeneous solution of the compound and first buffer, so that the nucleophilic groups on the core regain their nucleophilic character (that has been masked by the action of the acidic buffer), thus allowing the nucleophilic groups to inter-react with the electrophilic groups on the core.

An exemplary basic buffer is an aqueous solution of carbonate and phosphate salts. This buffer may be prepared by combining a base solution with a salt solution. The salt solution may be prepared by combining 34.7 g of monobasic sodium phosphate monohydrate, 49.3 g of sodium carbonate monohydrate, and sufficient water to provide a solution volume of 2 liter. Similarly, a 6 liter solution may be prepared by combining 104.0 g of monobasic sodium phosphate monohydrate, 147.94 g of sodium carbonate monohydrate, and sufficient water to provide 6 liter of the salt solution. The basic buffer may be prepared by combining 7.2 g of sodium hydroxide with 180.0 g of water. The basic buffer is typically prepared by adding the base solution as needed to the salt solution, ultimately to provide a mixture having the desired pH, e.g., a pH of 9.65 to 9.75.

In general, the basic species present in the basic buffer should be sufficiently basic to neutralize the acidity provided by the acidic buffer, but should not be so nucleophilic itself that it will react substantially with the electrophilic groups on the core. For this reason, relatively “soft” bases such as carbonate and phosphate are preferred in this embodiment of the invention.

To illustrate the preparation of a three-dimensional matrix of the present invention, one may combine an admixture of the self-reactive compound with a first, acidic, buffer (e.g., an acid solution, e.g., a dilute hydrochloric acid solution) to form a homogeneous solution. This homogeneous solution is mixed with a second, basic, buffer (e.g., a basic solution, e.g., an aqueous solution containing phosphate and carbonate salts) whereupon the reactive groups on the core of the self-reactive compound substantially immediately inter-react with one another to form a three-dimensional matrix.

Redox Reactive Groups:

In one embodiment of the invention, the reactive groups are vinyl groups such as styrene derivatives, which undergo a radical polymerization upon initiation with a redox initiator. The term “redox” refers to a reactive group that is susceptible to oxidation-reduction activation. The term “vinyl” refers to a reactive group that is activated by a redox initiator, and forms a radical upon reaction. X, Y and Z can be the same or different vinyl groups, for example, methacrylic groups.

For self-reactive compounds containing vinyl reactive groups, the initial environment typically will be an aqueous environment. The modification of the initial environment involves the addition of a redox initiator.

Oxidative Coupling Reactive Groups:

In one embodiment of the invention, the reactive groups undergo an oxidative coupling reaction. For example, X, Y and Z can be a halo group such as chloro, with an adjacent electron-withdrawing group on the halogen-bearing carbon (e.g., on the “L” linking group). Exemplary electron-withdrawing groups include nitro, aryl, and so forth.

For such reactive groups, the modification in the initial environment comprises a change in pH. For example, in the presence of a base such as KOH, the self-reactive compounds then undergo a de-hydro, chloro coupling reaction, forming a double bond between the carbon atoms, as illustrated below:

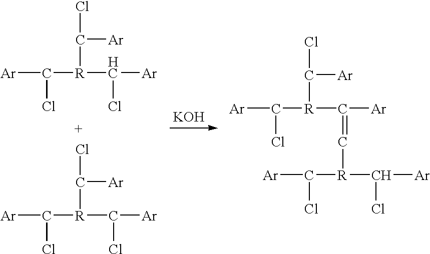

For self-reactive compounds containing oxidative coupling reactive groups, the initial environment typically can be can be dry and sterile, or a non-basic medium. The modification of the initial environment will typically comprise the addition of a base.

Photoinitiated Reactive Groups

In one embodiment of the invention, the reactive groups are photoinitiated groups. For such reactive groups, the modification in the initial environment comprises exposure to ultraviolet radiation.

In one embodiment of the invention, X can be an azide (—N₃) group and Y can be an alkyl group such as —CH(CH₃)₂ or vice versa. Exposure to ultraviolet radiation will then form a bond between the groups to provide for the following linkage: —NH—C(CH₃)₂—CH₂—. In another embodiment of the invention, X can be a benzophenone (—(C₆H₄)—C(O)—(C₆H₅)) group and Y can be an alkyl group such as —CH(CH₃)₂ or vice versa. Exposure to ultraviolet radiation will then form a bond between the groups to provide for the following linkage:

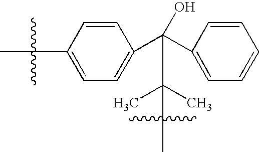

For self-reactive compounds containing photoinitiated reactive groups, the initial environment typically will be in an ultraviolet radiation-shielded environment. This can be for example, storage within a container that is impermeable to ultraviolet radiation.

The modification of the initial environment will typically comprise exposure to ultraviolet radiation.

Temperature-sensitive Reactive Groups:

In one embodiment of the invention, the reactive groups are temperature-sensitive groups, which undergo a thermochemical reaction. For such reactive groups, the modification in the initial environment thus comprises a change in temperature. The term “temperature-sensitive” refers to a reactive group that is chemically inert at one temperature or temperature range and reactive at a different temperature or temperature range.

In one embodiment of the invention, X, Y, and Z are the same or different vinyl groups.

For self-reactive compounds containing reactive groups that are temperature-sensitive, the initial environment typically will be within the range of about 10 to 30° C.

The modification of the initial environment will typically comprise changing the temperature to within the range of about 20 to 40° C.

Linking Groups:

The reactive groups may be directly attached to the core, or they may be indirectly attached through a linking group, with longer linking groups also termed “chain extenders.” In the formula (I) shown above, the optional linker groups are represented by L¹, L², and L³, wherein the linking groups are present when p, q and r are equal to 1.

Suitable linking groups are well known in the art. See, for example, WO 97/22371 to Rhee et al. Linking groups are useful to avoid steric hindrance problems that can sometimes associated with the formation of direct linkages between molecules. Linking groups may additionally be used to link several self-reactive compounds together to make larger molecules. In one embodiment, a linking group can be used to alter the degradative properties of the compositions after administration and resultant gel formation. For example, linking groups can be used to promote hydrolysis, to discourage hydrolysis, or to provide a site for enzymatic degradation.

Examples of linking groups that provide hydrolyzable sites, include, inter alia: ester linkages; anhydride linkages, such as those obtained by incorporation of glutarate and succinate; ortho ester linkages; ortho carbonate linkages such as trimethylene carbonate; amide linkages; phosphoester linkages; α-hydroxy acid linkages, such as those obtained by incorporation of lactic acid and glycolic acid; lactone-based linkages, such as those obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, WO 99/07417 to Coury et al. Examples of enzymatically degradable linkages include Leu-Gly-Pro-Ala, which is degraded by collagenase; and Gly-Pro-Lys, which is degraded by plasmin.

Linking groups can also be included to enhance or suppress the reactivity of the various reactive groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophilic group. By contrast, sterically bulky groups in the vicinity of a reactive group can be used to diminish reactivity and thus reduce the coupling rate as a result of steric hindrance.

By way of example, particular linking groups and corresponding formulas are indicated in the following Table 10:

TABLE 10

| Linking group | Component structure |
|---|---|
| —O—(CH₂)ₓ— | —O—(CH₂)ₓ—X |
| | —O—(CH₂)ₓ—Y |
| | —O—(CH₂)ₓ-Z |
| —S—(CH₂)ₓ— | —S—(CH₂)ₓ—X |
| | —S—(CH₂)ₓ—Y |
| | —S—(CH₂)ₓ-Z |
| —NH—(CH₂)ₓ— | —NH—(CH₂)ₓ—X |
| | —NH—(CH₂)ₓ—Y |
| | —NH—(CH₂)ₓ-Z |
| —O—(CO)—NH—(CH₂)ₓ— | —O—(CO)—NH—(CH₂)ₓ—X |
| | —O—(CO)—NH—(CH₂)ₓ—Y |
| | —O—(CO)—NH—(CH₂)ₓ-Z |
| —NH—(CO)—O—(CH₂)ₓ— | —NH—(CO)—O—(CH₂)ₓ—X |
| | —NH—(CO)—O—(CH₂)ₓ—Y |
| | —NH—(CO)—O—(CH₂)ₓ-Z |
| —O—(CO)—(CH₂)ₓ— | —O—(CO)—(CH₂)ₓ—X |
| | —O—(CO)—(CH₂)ₓ—Y |
| | —O—(CO)—(CH₂)ₓ-Z |
| —(CO)—O—(CH₂)ₓ— | —(CO)—O—(CH₂)ₙ—X |
| | —(CO)—O—(CH₂)ₙ—Y |
| | —(CO)—O—(CH₂)ₙ-Z |
| —O—(CO)—O—(CH₂)ₓ— | —O—(CO)—O—(CH₂)ₓ—X |
| | —O—(CO)—O—(CH₂)ₓ—Y |
| | —O—(CO)—O—(CH₂)ₓ-Z |
| —O—(CO)—CHR²— | —O—(CO)—CHR²—X |
| | —O—(CO)—CHR²—Y |
| | —O—(CO)—CHR²-Z |
| —O—R³—(CO)—NH— | —O—R³—(CO)—NH—X |
| | —O—R³—(CO)—NH—Y |
| | —O—R³—(CO)—NH-Z |

In the above Table, x is generally in the range of 1 to about 10; R² is generally hydrocarbyl, typically alkyl or aryl, preferably alkyl, and most preferably lower alkyl; and R³ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows. If a higher molecular weight self-reactive compound is to be used, it will preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Core:

The “core” of each self-reactive compound is comprised of the molecular structure to which the reactive groups are bound. The molecular core can a polymer, which includes synthetic polymers and naturally occurring polymers. In one embodiment, the core is a polymer containing repeating monomer units. The polymers can be hydrophilic, hydrophobic, or amphiphilic. The molecular core can also be a low molecular weight components such as a C₂₋₁₄ hydrocarbyl or a heteroatom-containing C₂₋₁₄ hydrocarbyl. The heteroatom-containing C₂₋₁₄ hydrocarbyl can have 1 or 2 heteroatoms selected from N, O and S. In a preferred embodiment, the self-reactive compound comprises a molecular core of a synthetic hydrophilic polymer.

Hydrophilic Polymers:

As mentioned above, the term “hydrophilic polymer” as used herein refers to a polymer having an average molecular weight and composition that naturally renders, or is selected to render the polymer as a whole “hydrophilic.” Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions.

Synthetic hydrophilic polymers may be homopolymers, block copolymers including di-block and tri-block copolymers, random copolymers, or graft copolymers. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments preferably degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Other biodegradable segments that may form part of the hydrophilic polymer core include polyesters such as polylactide, polyethers such as polyalkylene oxide, polyamides such as a protein, and polyurethanes. For example, the core of the self-reactive compound can be a diblock copolymer of tetrafunctionally activated polyethylene glycol and polylactide.

Synthetic hydrophilic polymers that are useful herein include, but are not limited to: polyalkylene oxides, particularly polyethylene glycol (PEG) and poly(ethylene oxide)-poly(propylene oxide) copolymers, including block and random copolymers; polyols such as glycerol, polyglycerol (PG) and particularly highly branched polyglycerol, propylene glycol; poly(oxyalkylene)-substituted diols, and poly(oxyalkylene)-substituted polyols such as mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; poly(acrylic acids) and analogs and copolymers thereof, such as polyacrylic acid per se, polymethacrylic acid, poly(hydroxyethylmethacrylate), poly(hydroxyethylacrylate), poly(methylalkylsulfoxide methacrylates), poly(methylalkylsulfoxide acrylates) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), poly(N-isopropyl-acrylamide), and copolymers thereof; poly(olefinic alcohols) such as poly(vinyl alcohols) and copolymers thereof; poly(N-vinyl lactams) such as poly(vinyl pyrrolidones), poly(N-vinyl caprolactams), and copolymers thereof; polyoxazolines, including poly(methyloxazoline) and poly(ethyloxazoline); and polyvinylamines; as well as copolymers of any of the foregoing. It must be emphasized that the aforementioned list of polymers is not exhaustive, and a variety of other synthetic hydrophilic polymers may be used, as will be appreciated by those skilled in the art.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Although a variety of different synthetic hydrophilic polymers can be used in the present compounds, preferred synthetic hydrophilic polymers are PEG and PG, particularly highly branched PG. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a PEG having a molecular weight within the range of about 100 to about 100,000, although for highly branched PEG, far higher molecular weight polymers can be employed, up to 1,000,000 or more, providing that biodegradable sites are incorporated ensuring that all degradation products will have a molecular weight of less than about 30,000. For most PEGs, however, the preferred molecular weight is about 1,000 to about 20,000, more preferably within the range of about 7,500 to about 20,000. Most preferably, the polyethylene glycol has a molecular weight of approximately 10,000.

Naturally occurring hydrophilic polymers include, but are not limited to: proteins such as collagen, fibronectin, albumins, globulins, fibrinogen, fibrin and thrombin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are preferred naturally occurring hydrophilic polymers for use herein.

Unless otherwise specified, the term “collagen” as used herein refers to all forms of collagen, including those, which have been processed or otherwise modified. Thus, collagen from any source may be used in the compounds of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. For example, U.S. Pat. No. 5,428,022 to Palefsky et al. discloses methods of extracting and purifying collagen from the human placenta, and U.S. Pat. No. 5,667,839 to Berg discloses methods of producing recombinant human collagen in the milk of transgenic animals, including transgenic cows.

Non-transgenic, recombinant collagen expression in yeast and other cell lines) is described in U.S. Pat. No. 6,413,742 to Olsen et al., U.S. Pat. No. 6,428,978 to Olsen et al., and U.S. Pat. No. 6,653,450 to Berg et al.

Collagen of any type, including, but not limited to, types I, II, III, IV, or any combination thereof, may be used in the compounds of the invention, although type I is generally preferred. Either atelopeptide or telopeptide-containing collagen may be used; however, when collagen from a natural source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the invention, although previously crosslinked collagen may be used.

Collagens for use in the present invention are generally, although not necessarily, in aqueous suspension at a concentration between about 20 mg/ml to about 120 mg/ml, preferably between about 30 mg/ml to about 90 mg/ml. Although intact collagen is preferred, denatured collagen, commonly known as gelatin, can also be used. Gelatin may have the added benefit of being degradable faster than collagen.

Nonfibrillar collagen is generally preferred for use in compounds of the invention, although fibrillar collagens may also be used. The term “nonfibrillar collagen” refers to any modified or unmodified collagen material that is in substantially nonfibrillar form, i.e., molecular collagen that is not tightly associated with other collagen molecules so as to form fibers. Typically, a solution of nonfibrillar collagen is more transparent than is a solution of fibrillar collagen. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559 to Miyata et al. Methylated collagen, which contains reactive amine groups, is a preferred nucleophile-containing component in the compositions of the present invention. In another aspect, methylated collagen is a component that is present in addition to first and second components in the matrix-forming reaction of the present invention. Methylated collagen is described in, for example, in U.S. Pat. No. 5,614,587 to Rhee et al.

Collagens for use in the compositions of the present invention may start out in fibrillar form, then can be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Fibrillar collagen is less preferred for use in the compounds of the invention. However, as disclosed in U.S. Pat. No. 5,614,587 to Rhee et al., fibrillar collagen, or mixtures of nonfibrillar and fibrillar collagen, may be preferred for use in compounds intended for long-term persistence in vivo.

Hydrophobic Polymers:

The core of the self-reactive compound may also comprise a hydrophobic polymer, including low molecular weight polyfunctional species, although for most uses hydrophilic polymers are preferred. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing, for example, multiple nucleophilic groups. Thus, use of short-chain oligomers can avoid solubility-related problems during reaction. Polylactic acid and polyglycolic acid are examples of two particularly suitable hydrophobic polymers.

Amphiphilic Polymers:

Generally, amphiphilic polymers have a hydrophilic portion and a hydrophobic (or lipophilic) portion. The hydrophilic portion can be at one end of the core and the hydrophobic portion at the opposite end, or the hydrophilic and hydrophobic portions may be distributed randomly (random copolymer) or in the form of sequences or grafts (block copolymer) to form the amphiphilic polymer core of the self-reactive compound. The hydrophilic and hydrophobic portions may include any of the aforementioned hydrophilic and hydrophobic polymers.

Alternately, the amphiphilic polymer core can be a hydrophilic polymer that has been modified with hydrophobic moieties (e.g., alkylated PEG or a hydrophilic polymer modified with one or more fatty chains ), or a hydrophobic polymer that has been modified with hydrophilic moieties (e.g., “PEGylated” phospholipids such as polyethylene glycolated phospholipids).

Low Molecular Weight Components:

As indicated above, the molecular core of the self-reactive compound can also be a low molecular weight compound, defined herein as being a C₂₋₁₄ hydrocarbyl or a heteroatom-containing C₂₋₁₄ hydrocarbyl, which contains 1 to 2 heteroatoms selected from N, O, S and combinations thereof. Such a molecular core can be substituted with any of the reactive groups described herein.

Alkanes are suitable C₂₋₁₄ hydrocarbyl molecular cores. Exemplary alkanes, for substituted with a nucleophilic primary amino group and a Y electrophilic group, include, ethyleneamine (H₂N—CH₂CH₂—Y), tetramethyleneamine (H₂N—(CH₄)—Y), pentamethyleneamine (H₂N—(CH₅)—Y), and hexamethyleneamine (H₂N—(CH₆)—Y).

Low molecular weight diols and polyols are also suitable C₂₋₁₄ hydrocarbyls and include trimethylolpropane, di(trimethylol propane), pentaerythritol, and diglycerol. Polyacids are also suitable C₂₋₁₄ hydrocarbyls, and include trimethylolpropane-based tricarboxylic acid, di(trimethylol propane)—based tetracarboxylic acid, heptanedioic acid, octanedioic acid (suberic acid), and hexadecanedioic acid (thapsic acid).

Low molecular weight diols and poly-electrophiles are suitable heteroatom-containing C₂₋₁₄ hydrocarbyl molecular cores. These include, for example, disuccinimidyl suberate (DSS), bis(sulfosuccinimidyl) suberate (BS₃), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives.

In one embodiment of the invention, the self-reactive compound of the invention comprises a low-molecular weight material core, with a plurality of acrylate moieties and a plurality of thiol groups.

Preparation:

The self-reactive compounds are readily synthesized to contain a hydrophilic, hydrophobic or amphiphilic polymer core or a low molecular weight core, functionalized with the desired functional groups, i.e., nucleophilic and electrophilic groups, which enable crosslinking. For example, preparation of a self-reactive compound having a polyethylene glycol (PEG) core is discussed below. However, it is to be understood that the following discussion is for purposes of illustration and analogous techniques may be employed with other polymers.

With respect to PEG, first of all, various functionalized PEGs have been used effectively in fields such as protein modification (see Abuchowski et al., Enzymes as Drugs, John Wiley & Sons: New York, N.Y. (1981) pp. 367-383; and Dreborg et al. (1990) Crit. Rev. Therap. Drug Carrier Syst. 6:315), peptide chemistry (see Mutter et al., The Peptides, Academic: New York, N.Y. 2:285-332; and Zalipsky et al. (1987) Int. J. Peptide Protein Res. 30:740), and the synthesis of polymeric drugs (see Zalipsky et al. (1983) Eur. Polym. J. 19:1177; and Ouchi et al. (1987) J. Macromol. Sci. Chem. A24:1011).

Functionalized forms of PEG, including multi-functionalized PEG, are commercially available, and are also easily prepared using known methods. For example, see Chapter 22 of Poly(ethylene Glycol)Chemistry: Biotechnical and Biomedical Applications, J. Milton Harris, ed., Plenum Press, NY (1992).

Multi-functionalized forms of PEG are of particular interest and include, PEG succinimidyl glutarate, PEG succinimidyl propionate, succinimidyl butylate, PEG succinimidyl acetate, PEG succinimidyl succinamide, PEG succinimidyl carbonate, PEG propionaldehyde, PEG glycidyl ether, PEG-isocyanate, and PEG-vinylsulfone. Many such forms of PEG are described in U.S. Pat. Nos. 5,328,955 and 6,534,591, both to Rhee et al. Similarly, various forms of multi-amino PEG are commercially available from sources such as PEG Shop, a division of SunBio of South Korea (www.sunbio.com), Nippon Oil and Fats (Yebisu Garden Place Tower, 20-3 Ebisu 4-chome, Shibuya-ku, Tokyo), Nektar Therapeutics (San Carlos, Calif., formerly Shearwater Polymers, Huntsville, Ala.) and from Huntsman's Performance Chemicals Group (Houston, Tex.) under the name Jeffamine® polyoxyalkyleneamines. Multi-amino PEGs useful in the present invention include the Jeffamine diamines (“D” series) and triamines (“T” series), which contain two and three primary amino groups per molecule. Analogous poly(sulfhydryl) PEGs are also available from Nektar Therapeutics, e.g., in the form of pentaerythritol poly(ethylene glycol)ether tetra-sulfhydryl (molecular weight 10,000). These multi-functionalized forms of PEG can then be modified to include the other desired reactive groups.

Reaction with succinimidyl groups to convert terminal hydroxyl groups to reactive esters is one technique for preparing a core with electrophilic groups. This core can then be modified include nucleophilic groups such as primary amines, thiols, and hydroxyl groups. Other agents to convert hydroxyl groups include carbonyldiimidazole and sulfonyl chloride. However, as discussed herein, a wide variety of electrophilic groups may be advantageously employed for reaction with corresponding nucleophilic groups. Examples of such electrophilic groups include acid chloride groups; anhydrides, ketones, aldehydes, isocyanate, isothiocyanate, epoxides, and olefins, including conjugated olefins such as ethenesulfonyl (—SO₂CH═CH₂) and analogous functional groups.

Other in situ Crosslinking Materials

Numerous other types of in situ forming materials have been described which may be used in combination with an anti-scarring agent in accordance with the invention. The in situ forming material may be a biocompatible crosslinked polymer that is formed from water soluble precursors having electrophilic and nucleophilic groups capable of reacting and crosslinking in situ (see, e.g., U.S. Pat. No. 6,566,406). The in situ forming material may be hydrogel that may be formed through a combination of physical and chemical crosslinking processes, where physical crosslinking is mediated by one or more natural or synthetic components that stabilize the hydrogel-forming precursor solution at a deposition site for a period of time sufficient for more resilient chemical crosslinks to form (see, e.g., U.S. Pat. No. 6,818,018). The in situ forming material may be formed upon exposure to an aqueous fluid from a physiological environment from dry hydrogel precursors (see, e.g., U.S. Pat. No. 6,703,047). The in situ forming material may be a hydrogel matrix that provides controlled release of relatively low molecular weight therapeutic species by first dispersing or dissolving the therapeutic species within relatively hydrophobic rate modifying agents to form a mixture; the mixture is formed into microparticles that are dispersed within bioabsorbable hydrogels, so as to release the water soluble therapeutic agents in a controlled fashion (see, e.g., U.S. Pat. No. 6,632,457). The in situ forming material may be a multi-component hydrogel system (see, e.g., U.S. Pat. No. 6,379, 373). The in situ forming material may be a multi-arm block copolymer that includes a central core molecule, such as a residue of a polyol, and at least three copolymer arms covalently attached to the central core molecule, each copolymer arm comprising an inner hydrophobic polymer segment covalently attached to the central core molecule and an outer hydrophilic polymer segment covalently attached to the hydrophobic polymer segment, wherein the central core molecule and the hydrophobic polymer segment define a hydrophobic core region (see, e.g., U.S. Pat. No. 6,730,334). The in situ forming material may include a gel-forming macromer that includes at least four polymeric blocks, at least two of which are hydrophobic and at least one of which is hydrophilic, and including a crosslinkable group (see, e.g., U.S. Pat. No. 6,639,014). The in situ forming material may be a water-soluble macromer that includes at least one hydrolysable linkage formed from carbonate or dioxanone groups, at least one water-soluble polymeric block, and at least one polymerizable group (see, e.g., U.S. Pat. No. 6,177,095). The in situ forming material may comprise polyoxyalkylene block copolymers that form weak physical crosslinks to provide gels having a paste-like consistency at physiological temperatures. (see, e.g., U.S. Pat. No. 4,911,926). The in situ forming material may be a thermo-irreversible gel made from polyoxyalkylene polymers and ionic polysaccharides (see, e.g., U.S. Pat. No. 5,126,141). The in situ forming material may be a gel forming composition that includes chitin derivatives (see, e.g., U.S. Pat. No. 5,093,319), chitosan-coagulum (see, e.g., U.S. Pat. No. 4,532,134), or hyaluronic acid (see, e.g., U.S. Pat. No. 4,141,973). The in situ forming material may be an in situ modification of alginate (see, e.g., U.S. Pat. No. 5,266,326). The in situ forming material may be formed from ethylenically unsaturated water soluble macromers that can be crosslinked in contact with tissues, cells, and bioactive molecules to form gels (see, e.g., U.S. Pat. No. 5,573,934). The in situ forming material may include urethane prepolymers used in combination with an unsaturated cyano compound containing a cyano group attached to a carbon atom, such as cyano(meth)acrylic acids and esters thereof (see, e.g., U.S. Pat. No. 4,740,534). The in situ forming material may be a biodegradable hydrogel that polymerizes by a photoinitiated free radical polymerization from water soluble macromers (see, e.g., U.S. Pat. No. 5,410,016). The in situ forming material may be formed from a two component mixture including a first part comprising a serum albumin protein in an aqueous buffer having a pH in a range of about 8.0-11.0, and a second part comprising a water-compatible or water-soluble bifunctional crosslinking agent. (see, e.g., U.S. Pat. No. 5,583,114).

In another aspect, in situ forming materials that can be used include those based on the crosslinking of proteins. For example, the in situ forming material may be a biodegradable hydrogel composed of a recombinant or natural human serum albumin and poly(ethylene) glycol polymer solution whereby upon mixing the solution cross-links to form a mechanical non-liquid covering structure which acts as a sealant. See e.g., U.S. Pat. No. 6,458,147 and 6,371,975. The in situ forming material may be composed of two separate mixtures based on fibrinogen and thrombin which are dispensed together to form a biological adhesive when intermixed either prior to or on the application site to form a fibrin sealant. See e.g., U.S. Pat. No. 6,764,467. The in situ forming material may be composed of ultrasonically treated collagen and albumin which form a viscous material that develops adhesive properties when crosslinked chemically with glutaraldehyde and amino acids or peptides. See e.g., U.S. Pat. No. 6,310,036. The in situ forming material may be a hydrated adhesive gel composed of an aqueous solution consisting essentially of a protein having amino groups at the side chains (e.g., gelatin, albumin) which is crosslinked with an N-hydroxyimidoester compound. See e.g., U.S. Pat. No. 4,839,345. The in situ forming material may be a hydrogel prepared from a protein or polysaccharide backbone (e.g., albumin or polymannuronic acid) bonded to a cross-linking agent (e.g., polyvalent derivatives of polyethylene or polyalkylene glycol). See e.g., U.S. Pat. No. 5,514,379. The in situ forming material may be composed of a polymerizable collagen composition that is applied to the tissue and then exposed to an initiator to polymerize the collagen to form a seal over a wound opening in the tissue. See e.g., U.S. Pat. No. 5,874,537. The in situ forming material may be a two component mixture composed of a protein (e.g., serum albumin) in an aqueous buffer having a pH in the range of about 8.0-11.0 and a water-soluble bifunctional polyethylene oxide type crosslinking agent, which transforms from a liquid to a strong, flexible bonding composition to seal tissue in situ. See e.g., U.S. Pat. No. 5,583,114 and RE38158 and PCT Publication No. WO 96/03159. The in situ forming material may be composed of a protein, a surfactant, and a lipid in a liquid carrier, which is crosslinked by adding a crosslinker and used as a sealant or bonding agent in situ. See e.g., U.S. Pat. Application No. 2004/0063613A1 and PCT Publication Nos. WO 01/45761 and WO 03/090683. The in situ forming material may be composed of two enzyme-free liquid components that are mixed by dispensing the components into a catheter tube deployed at the vascular puncture site, wherein, upon mixing, the two liquid components chemically cross-link to form a mechanical non-liquid matrix that seals a vascular puncture site. See e.g., U.S. Patent Application Nos. 2002/0161399A1 and 2001/0018598A1. The in situ forming material may be a cross-linked albumin composition composed of an albumin preparation and a carbodiimide preparation which are mixed under conditions that permit crosslinking of the albumin for use as a bioadhesive or sealant. See e.g., PCT Publication No. WO 99/66964. The in situ forming material may be composed of collagen and a peroxidase and hydrogen peroxide, such that the collagen is crosslinked to from a semi-solid gel that seals a wound. See e.g., PCT Publication No. WO 01/35882.

In another aspect, in situ forming materials that can be used include those based on isocyanate or isothiocyanate capped polymers. For example, the in situ forming material may be composed of isocyanate-capped polymers that are liquid compositions which form into a solid adhesive coating by in situ polymerization and crosslinking upon contact with body fluid or tissue. See e.g., PCT Publication No. WO 04/021983. The in situ forming material may be a moisture-curing sealant composition composed of an active isocyanato-terminated isocyanate prepolymer containing a polyol component with a molecular weight of 2,000 to 20,000 and an isocyanurating catalyst agent. See e.g., U.S. Pat. No. 5,206,331.

In another embodiment, the reagents can undergo an electrophilic-nucleophilic reaction to produce a crosslinked matrix. Polymers containing and/or terminated with nucleophilic groups such as amine, sulfhydryl, hydroxyl, —PH₂ or CO—NH—NH₂ can be used as the nucleophilic reagents and polymers containing and/or terminated with electrophilic groups such as succinimidyl, carboxylic acid, aldehyde, epoxide, isocyanate, vinyl, vinyl sulfone, maleimide, —S—S—(C₅H₄N) or activated esters, such as are used in peptide synthesis can be used as the electrophilic reagents. For example, a 4-armed thiol derivatized poly(ethylene glycol) (e.g., pentaerythritol poly(ethylene glycol)ether tetra-sulfhydryl) can be reacted with a 4 armed NHS-derivatized polyethylene glycol (e.g., pentaerythritol poly(ethylene glycol)ether tetra-succinimidyl glutarate) under basic conditions (pH>about 8). Representative examples of compositions that undergo such electrophilic-nucleophilic crosslinking reactions are described, for example, in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; and PCT Application Publication Nos. WO 04/060405 and WO 04/060346.

In another embodiment, the electrophilic- or nucleophilic-terminated polymers can further comprise a polymer that can enhance the mechanical and/or adhesive properties of the in situ forming compositions. This polymer can be a degradable or non-degradable polymer. For example, the polymer may be collagen or a collagen derivative, for example methylated collagen. An example of an in situ forming composition uses pentaerythritol poly(ethylene glycol)ether tetra-sulfhydryl) (4-armed thiol PEG), pentaerythritol poly(ethylene glycol)ether tetra-succinimidyl glutarate) (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another embodiment, the reagents that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In the preferred embodiment, the 4 armed NHS-derivatized polyethylene glycol is applied to the tissue under basic conditions (pH>about 8). Other representative examples of compositions of this nature that may be used are disclosed in PCT Application Publication No. WO 04/060405 and WO 04/060346, and U.S. Patent Application No. 10/749,123.

In another embodiment, the in situ forming material polymer can be a polyester. Polyesters that can be used in in situ forming compositions include poly(hydroxyesters). In another embodiment, the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Representative examples of these types of compositions are described in U.S. Pat. Nos. 5,874,500; 5,936,035; 6,312,725; 6,495,127 and PCT Publication Nos. WO 2004/028547.

In another embodiment, the electrophilic-terminated polymer can be partially or completely replaced by a small molecule or oligomer that comprises an electrophilic group (e.g., disuccinimidyl glutarate).

In another embodiment, the nucleophilic-terminated polymer can be partially or completely replaced by a small molecule or oligomer that comprises a nucleophilic group (e.g., dicysteine, dilysine, trilysine, etc.).

Other examples of in situ forming materials that can be used include those based on the crosslinking of proteins (described in, for example, U.S. Pat. Nos. RE38158; 4,839,345; 5,514,379, 5,583,114; 6,310,036; 6,458,147; 6,371,975; US Patent Application Publication Nos. 2004/0063613A1, 2002/0161399A1, and 2001/0018598A1, and PCT Publication Nos. WO 03/090683, WO 01/45761, WO 99/66964, and WO 96/03159) and those based on isocyanate or isothiocyanate capped polymers (see, e.g., PCT Publication No. WO 04/021983).

Other examples of in situ forming materials can include reagents that comprise one or more cyanoacrylate groups. These reagents can be used to prepare a poly(alkylcyanoacrylate) or poly(carboxyalkylcyanoacrylate) (e.g., poly(ethylcyanoacrylate), poly(butylcyanoacrylate), poly(isobutylcyanoacrylate), poly(hexylcyanoacrylate), poly(methoxypropylcyanoacrylate), and poly(octylcyanoacrylate)).

Examples of commercially available cyanoacrylates that can be used in the present invention include DERMABOND, INDERMIL, GLUSTITCH, VETBOND, HISTOACRYL, TISSUMEND, HISTOACRYL BLUE and ORABASE SOOTHE-N-SEAL LIQUID PROTECTANT.

In another embodiment, the cyanoacrylate compositions may further comprise additives to stabilize the reagents and/or alter the rate of reaction of the cyanoacrylate, and/or plasticize the poly(cyanoacrylate), and/or alter the rate of degradation of the poly(cyanoacrylate). For example, a trimethylene carbonate based polymer or an oxalate polymer of poly(ethylene glycol) or a ε-caprolactone based copolymer may be mixed with a 2-alkoxyalkylcyanoacrylate (e.g., 2-methoxypropylcyanoacrylate). Representative examples of these compositions are described in U.S. Pat. Nos. 5,350,798 and 6,299,631.

In another embodiment, the cyanoacrylate composition can be prepared by capping heterochain polymers with a cyanoacrylate group. The cyanoacrylate-capped heterochain polymer preferably has at least two cyanoacrylate ester groups per chain. The heterochain polymer can comprise an absorbable poly(ester), poly(ester-carbonate), poly(ether-carbonate) and poly(ether-ester). The poly(ether-ester)s described in U.S. Pat. Nos. 5,653,992 and 5,714,159 can also be used as the heterochain polymers. A triaxial poly(ε-caprolactone-co-trimethylene carbonate) is an example of a poly(ester-carbonate) that can be used. The heterochain polymer may be a polyether. Examples of polyethers that can be used include poly(ethylene glycol), poly(propylene glycol) and block copolymers of poly(ethylene glycol) and poly(propylene glycol) (e.g., PLURONICS group of polymers including but not limited to PLURONIC F127 or F68). Representative examples of these compositions are described in U.S. Pat. No. 6,699,940.

Within another aspect of the invention, fibrosis-inhibiting drug combinations (or individual components thereof) can be delivered with a non-polymeric compound (e.g., a carrier). These non-polymeric carriers can include sucrose derivatives (e.g., sucrose acetate isobutyrate, sucrose oleate), sterols such as cholesterol, stigmasterol, β-sitostetol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Within certain embodiments of the invention, the therapeutic compositions are provided that include a fibrosis-inhibiting drug combination (or individual component(s) thereof). The therapeutic compositions may include one or more additional therapeutic agents (such as described above), for example, anti-inflammatory agents, anti-thrombotic agents, and/ or anti-platelet agents. Other agents that may be combined with the therapeutic compositions include, e.g., additional ingredients such as surfactants (e.g., PLURONICS, such as F-127, L-122, L-101, L-92, L-81, and L-61), preservatives, anti-oxidants.

In certain embodiments, the present invention provides compositions comprising i) an anti-fibrotic drug combination and ii) a polymer or a compound that forms a polymer in situ. The following are some, but by no means all, of the anti-fibrotic drug combinations that may be included in the inventive compositions:

1a. amoxapine and prednisolone,

2a. paroxetine and prednisolone,

3a. dipyridamole and prednisolone,

4a. dexamethasone and econazole,

5a. diflorasone and alprostadil,

6a. dipyridamole and amoxapine,

7a. dipyridamole and ibudilast,

8a. nortriptyline and loratadine (or desloratadine),

9a. albendazole and pentamidine,

10a. itraconazole and lovastatin,

11a. terbinafine and manganese sulfate,

12a. (1) a triazole (e.g., fluconazole or itraconazole) and (2) a diaminopyridine (e.g., phenazopyridine (PZP), phenothiazine, dacarbazine, phenelzine);

13a. (1) an antiprotozoal (e.g., pentamidine) and (2) a diaminopyridine (e.g., phenazopyridine) or a quaternary ammonium compound (e.g., pentolinium);

14a. (1) an aromatic diamidine and (2) one selected from the group consisting of: (a) an antiestrogen, (b) an anti-fungal imidazole, (d) disulfiram, (e) ribavirin, (f) (i) aminopyridine and (ii) phenothiazine, dacarbazine, or phenelzine, (g) (i) a quaternary ammonium compound and (ii) an anti-fungal imidazole, halopnogin, MnSO₄, or ZnCl₂, (h) (i) an antiestrogen and (ii) phenothiazine, cupric chloride, dacarbazine, methoxsalen, or phenelzine, (j) (i) an antifungal imidazone and (ii) disulfiram or ribavirin, and (k) an estrogenic compound and (ii) dacarbazine;

15a. (1) amphotericin B and (2) dithiocarbamoyl disulfide (e.g., disulfiram);

16a. (1) terbinafine and (2) a manganese compound;

17a. (1) a tricyclic antidepreseant (TCA) (e.g., amoxapine) and (2) a corticosteroid (e.g., prednisolone, glucocorticoid, mineralocorticoid);

18a. (1) a tetra-substituted pyrimidopyrimidine (e.g., dipyridamole) and (2) a corticosteroid (e.g., fludrocortisone or prednisolone);

19a. (1) a prostaglandin (e.g., alprostadil) and (2) a retinoid (e.g., tretinoin (vitamin A));

20a. (1) an azole (e.g., imidazone or triazole) and (2) a steroid (e.g., corticosteroids including glucocorticoid or mineralocorticoid);

21a. (1) a steroid and (2) a prostaglandin, beta-adrenergic receptor ligand, anti-mitotic agent, or microtubule inhibitor;

22a. (1) a serotonin norepinephrine reuptake inhibitor (SNRI) or naradrenaline reuptake inhibitor (NARI) and (2) a corticosteroid;

23a. (1) a non-steroidal immunophilin-dependent immunosuppressant (NSIDI) (e.g., calcineurin inhibitor including cyclosporin, tacrolimus, ascomycin, pimecrolimus, ISAtx 247) and (2) a non-steroidal immunophilin-dependent immunosuppressant enhancer (NSIDIE) (e.g., selective serotonin reuptake inhibitors, tricyclic antidepressants, phenoxy phenols, anti-histamine, phenothiazines, or mu opioid receptor agonists);

24a. (1) an antihistamines and (2) an additional agent selected from corticosteroids, tricyclic or tetracyclic antidepressants, selective serotonin reuptake inhibitors, and steroid receptor modulators;

25a. (1) a tricyclic compound and (2) a corticosteroid;

26a. (1) an antipsychotic drug (e.g., chlorpromazine) and (2) an antiprotozoal drug (e.g., pentamidine);

27a. (1) an antihelmintic drug (e.g., benzimidazole) and (2) an antiprotozoal drug (e.g., pentamidine);

28a. (1) ciclopirox and (2) an antiproliferative agent;

29a. (1) a salicylanilide (e.g., niclosamide) and (2) an antriproliferative agents;

30a. (1) pentamidine or its analogue and (2) chlorpromazine or its analogue;

31a. (1) an antihelmintic drug (e.g., alberdazole, mebendazole, oxibendazole) and (2) an antiprotozoal drug (e.g., pentamidine);

32a. (1) a dibucaine or amide local anaesthetic related to bupivacaine and (2) a vinca alkaloid;

33a. (1) pentamidine, analogue or metabolite thereof and (2) an antiproliferative agent;

34a. (1) a triazole (e.g., itraconazole) and (2) an antiarrhythmic agents (e.g., amiodarone, nicardipine or bepridil);

35a. (1) an azole and (2) an HMG-CoA reductase inhibitor;

36a. a phenothiazine conjugate (e.g., a conjugate of phenothiazine and an antiproliferative agent;

37a. (1) phenothiazine and (2) an antiproliferative agent;

38a. (1) a kinesin inhibitor (e.g., phenothiazine, analog or metabolite) and (2) an antiproliferative agent (e.g., Group A and Group B antiproliferative agents);

39a. (1) an agent that reduces the biological activity of a mitotic kinesin (e.g., chlorpromazine) and (2) an agent that reduces the biological activity of protein tyrosine phosphatase.

As mentioned above, the present invention provides compositions comprising each of the foregoing 39 (i. e., 1a through 39a) listed anti-fibrotic drug combinations or classes of anti-fibrotic drug combinations, with each of the following 97 (i.e., 1b through 97b) polymers and compounds:

1b. A crosslinked polymer.

2b. A polymer that reacts with mammalian tissue.

3b. A polymer that is a naturally occurring polymer.

4b. A polymer that is a protein.

5b. A polymer that is a carbohydrate.

6b. A polymer that is biodegradable.

7b. A polymer that is crosslinked and biodegradable.

8b. A polymer that nonbiodegradable.

9b. Collagen.

10b. Methylated collagen.

11b. Fibrinogen.

12b. Thrombin.

13b. Albumin.

14b. Plasminogen.

15b. von Willebrands factor.

16b. Factor VIII.

17b. Hypoallergenic collagen.

18b. Atelopeptidic collagen.

19b. Telopeptide collagen.

20b. Crosslinked collagen.

21b. Aprotinin.

22b. Gelatin.

23b. A protein conjugate.

24b. A gelatin conjugate.

25b. Hyaluronic acid.

26b. A hyaluronic acid derivative.

27b. A synthetic polymer.

28b. A polymer formed from reactants comprising a synthetic isocyanate-containing compound.

29b. A synthetic isocyanate-containing compound.

30b. A polymer formed from reactants comprising a synthetic thiol-containing compound.

31b. A synthetic thiol-containing compound.

32b. A polymer formed from reactants comprising a synthetic compound containing at least two thiol groups.

33b. A synthetic compound containing at least two thiol groups.

34b. A polymer formed from reactants comprising a synthetic compound containing at least three thiol groups.

35b. A synthetic compound containing at least three thiol groups.

36b. A polymer formed from reactants comprising a synthetic compound containing at least four thiol groups.

37b. A synthetic compound containing at least four thiol groups.

38b. A polymer formed from reactants comprising a synthetic amino-containing compound.

39b. A synthetic amino-containing compound.

40b. A polymer formed from reactants comprising a synthetic compound containing at least two amino groups.

41b. A synthetic compound containing at least two amino groups.

42b. A polymer formed from reactants comprising a synthetic compound containing at least three amino groups.

43b. A synthetic compound containing at least three amino groups.

44b. A polymer formed from reactants comprising a synthetic compound containing at least four amino groups.

45b. A synthetic compound containing at least four amino groups.

46b. A polymer formed from reactants comprising a synthetic compound comprising a carbonyl-oxygen-succinimidyl group.

47b. A synthetic compound comprising a carbonyl-oxygen-succinimidyl group.

48b. A polymer formed from reactants comprising a synthetic compound comprising at least two carbonyl-oxygen-succinimidyl groups.

49b. A synthetic compound comprising at least two carbonyl-oxygen-succinimidyl groups.

50b. A polymer formed from reactants comprising a synthetic compound comprising at least three carbonyl-oxygen-succinimidyl groups.

51b. A synthetic compound comprising at least three carbonyl-oxygen-succinimidyl groups.

52b. A polymer formed from reactants comprising a synthetic compound comprising at least four carbonyl-oxygen-succinimidyl groups.

53b. A synthetic compound comprising at least four carbonyl-oxygen-succinimidyl groups.

54b. A polymer formed from from reactants comprising a synthetic polyalkylene oxide-containing compound.

55b. A synthetic polyalkylene oxide-containing compound.

56b. A polymer formed from reactants comprising a synthetic compound comprising both polyalkylene oxide and biodegradable polyester blocks.

57b. A synthetic compound comprising both polyalkylene oxide and biodegradable polyester blocks.

58b. A polymer formed from reactants comprising a synthetic polyalkylene oxide-containing compound having reactive amino groups.

59b. A synthetic polyalkylene oxide-containing compound having reactive amino groups.

60b. A polymer formed from reactants comprising a synthetic polyalkylene oxide-containing compound having reactive thiol groups.

61b. A synthetic polyalkylene oxide-containing compound having reactive thiol groups.

62b. A polymer formed from reactants comprising a synthetic polyalkylene oxide-containing compound having reactive carbonyl-oxygen-succinimidyl groups.

63b. A synthetic polyalkylene oxide-containing compound having reactive carbonyl-oxygen-succinimidyl groups.

64b. A polymer formed from reactants comprising a synthetic compound comprising a biodegradable polyester block.

65b. A synthetic compound comprising a biodegradable polyester block.

66b. A polymer formed from reactants comprising a synthetic polymer formed in whole or part from lactic acid or lactide.

67b. A synthetic polymer formed in whole or part from lactic acid or lactide.

68b. A polymer formed from reactants comprising a synthetic polymer formed in whole or part from glycolic acid or glycolide.

69b. A synthetic polymer formed in whole or part from glycolic acid or glycolide.

70b. A polymer formed from reactants comprising polylysine.

71b. Polylysine.

72b. A polymer formed from reactants comprising (a) protein and (b) a compound comprising a polyalkylene oxide portion.

73b. A polymer formed from reactants comprising (a) protein and (b) polylysine.

74b. A polymer formed from reactants comprising (a) protein and (b) a compound having at least four thiol groups.

75b. A polymer formed from reactants comprising (a) protein and (b) a compound having at least four amino groups.

76b. A polymer formed from reactants comprising (a) protein and (b) a compound having at least four carbonyl-oxygen-succinimide groups.

77b. A polymer formed from reactants comprising (a) protein and (b) a compound having a biodegradable region formed from reactants selected from lactic acid, lactide, glycolic acid, glycolide, and epsilon-caprolactone.

78b. A polymer formed from reactants comprising (a) collagen and (b) a compound comprising a polyalkylene oxide portion.

79b. A polymer formed from reactants comprising (a) collagen and (b) polylysine.

80b. A polymer formed from reactants comprising (a) collagen and (b) a compound having at least four thiol groups.

81b. A polymer formed from reactants comprising (a) collagen and (b) a compound having at least four amino groups.

82b. A polymer formed from reactants comprising (a) collagen and (b) a compound having at least four carbonyl-oxygen-succinimide groups.

83b. A polymer formed from reactants comprising (a) collagen and (b) a compound having a biodegradable region formed from reactants selected from lactic acid, lactide, glycolic acid, glycolide, and epsilon-caprolactone.

84b. A polymer formed from reactants comprising (a) methylated collagen and (b) a compound comprising a polyalkylene oxide portion.

85b. A polymer formed from reactants comprising (a) methylated collagen and (b) polylysine.

86b. A polymer formed from reactants comprising (a) methylated collagen and (b) a compound having at least four thiol groups.

87b. A polymer formed from reactants comprising (a) methylated collagen and (b) a compound having at least four amino groups.

88b. A polymer formed from reactants comprising (a) methylated collagen and (b) a compound having at least four carbonyl-oxygen-succinimide groups.

89b. A polymer formed from reactants comprising (a) methylated collagen and (b) a compound having a biodegradable region formed from reactants selected from lactic acid, lactide, glycolic acid, glycolide, and epsilon-caprolactone.

90b. A polymer formed from reactants comprising hyaluronic acid.

91b. A polymer formed from reactants comprising a hyaluronic acid derivative.

92b. A polymer formed from reactants comprising pentaerythritol poly(ethylene glycol)ether tetra-sulfhydryl of number average molecular weight between 3,000 and 30,000.

93b. Pentaerythritol poly(ethylene glycol)ether tetra-sulfhydryl of number average molecular weight between 3,000 and 30,000.

94b. A polymer formed from reactants comprising pentaerythritol poly(ethylene glycol)ether tetra-amino of number average molecular weight between 3,000 and 30,000.

95b. Pentaerythritol poly(ethylene glycol)ether tetra-amino of number average molecular weight between 3,000 and 30,000.

96b. A polymer formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

97b. A mixture of (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

As mentioned above, the present invention provides compositions comprising each of the foregoing 39 (1a through 39a) listed anti-fibrotic agents or classes of anti-fibrotic agents, with each of the foregoing 97 (1b through 97b) polymers and compounds. Thus, in separate aspects, the invention provides 39 times 97=3,783 described compositions. In other words, each of the following is a distinct aspect of the present invention: 1a+1b; 1a+2b; 1a+3b; 1a+4b; 1a+5b; 1a+6b; 1a+7b; 1a+8b; 1a+9b; 1a+10b; 1a+11b; 1a+12b; 1a+13b; 1a+14b; 1a+15b; 1a+16b; 1a+17b; 1a+18b; 1a+19b; 1a+20b; 1a+21b; 1a+22b; 1a+23b; 1a+24b; 1a+25b; 1a+26b; 1a+27b; 1a+28b; 1a+29b; 1a+30b; 1a+31b; 1a+32b; 1a+33b; 1a+34b; 1a+35b; 1a+36b; 1a+37b; 1a+38b; 1a+39b; 1a+40b; 1a+41b; 1a+42b; 1a+43b; 1a+44b; 1a+45b; 1a+46b; 1a+47b; 1a+48b; 1a+49b; 1a+50b; 1a+51b; 1a+52b; 1a+53b; 1a+54b; 1a+55b; 1a+55b; 1a+57b; 1a+58b; 1a+59b; 1a+60b; 1a+61b; 1a+62b; 1a+63b; 1a+64b; 1a+65b; 1a+66b; 1a+67b; 1a+68b; 1a+69b; 1a+70b; 1a+71b; 1a+72b; 1a+73b; 1a+74b; 1a+75b; 1a+76b; 1a+77b; 1a+78b; 1a+79b; 1a+80b; 1a+81b; 1a+82b; 1a+83b; 1a+84b; 1a+85b; 1a+86b; 1a+87b; 1a+88b; 1a+89b; 1a+90b; 1a+91b; 1a+92b; 1a+93b; 1a+94b; 1a+95b; 1a+96b; 1a+97b; 2a+1b; 2a+2b; 2a+3b; 2a+4b; 2a+5b; 2a+6b; 2a+7b; 2a+8b; 2a+9b; 2a+10b; 2a+11b; 2a+12b; 2a+13b; 2a+14b; 2a+15b; 2a+16b; 2a+17b; 2a+18b; 2a+19b; 2a+20b; 2a+21b; 2a+22b; 2a+23b; 2a+24b; 2a+25b; 2a+26b; 2a+27b; 2a+28b; 2a+29b; 2a+30b; 2a+31b; 2a+32b; 2a+33b; 2a+34b; 2a+35b; 2a+36b; 2a+37b; 2a+38b; 2a+39b; 2a+40b; 2a+41b; 2a+42b; 2a+43b; 2a+44b; 2a+45b; 2a+46b; 2a+47b; 2a+48b; 2a+49b; 2a+50b; 2a+51b; 2a+52b; 2a+53b; 2a+54b; 2a+55b; 2a+55b; 2a+57b; 2a+58b; 2a+59b; 2a+60b; 2a+61b; 2a+62b; 2a+63b; 2a+64b; 2a+65b; 2a+66b; 2a+67b; 2a+68b; 2a+69b; 2a+70b; 2a+71b; 2a+72b; 2a+73b; 2a+74b; 2a+75b; 2a+76b; 2a+77b; 2a+78b; 2a+79b; 2a+80b; 2a+81b; 2a+82b; 2a+83b; 2a+84b; 2a+85b; 2a+86b; 2a+87b; 2a+88b; 2a+89b; 2a+90b; 2a+91b; 2a+92b; 2a+93b; 2a+94b; 2a+95b; 2a+96b; 2a+97b; 3a+22b; 3a+23b; 3a+24b; 3a+25b; 3a+26b; 3a+27b; 3a+28b; 3a+29b; 3a+30b; 3a+31b; 3a+32b; 3a+33b; 3a+34b; 3a+35b; 3a+36b; 3a+37b; 3a+38b; 3a+39b; 3a+40b; 3a+41b; 3a+42b; 3a+43b; 3a+44b; 3a+45b; 3a+46b; 3a+47b; 3a+48b; 3a+49b; 3a+50b; 3a+51b; 3a+52b; 3a+53b; 3a+54b; 3a+55b; 3a+55b; 3a+57b; 3a+58b; 3a+59b; 3a+60b; 3a+61b; 3a+62b; 3a+63b; 3a+64b; 3a+65b; 3a+66b; 3a+67b; 3a+68b; 3a+69b; 3a+70b; 3a+71b; 3a+72b; 3a+73b; 3a+74b; 3a+75b; 3a+76b; 3a+77b; 3a+78b; 3a+79b; 3a+80b; 3a+81b; 3a+82b; 3a+83b; 3a+84b; 3a+85b; 3a+86b; 3a+87b; 3a+88b; 3a+89b; 3a+90b; 3a+91b; 3a+92b; 3a+93b; 3a+94b; 3a+95b; 3a+96b; 3a+97b; 30 4a+12b; 4a+13b; 4a+14b; 4a+15b; 4a+16b; 4a+17b; 4a+18b; 4a+19b; 4a+20b; 4a+21b; 4a+22b; 4a+23b; 4a+24b; 4a+25b; 4a+26b; 4a+27b; 4a+28b; 4a+29b; 4a+30b; 4a+31b; 4a+32b; 4a+33b; 4a+34b; 4a+35b; 4a+36b; 4a+37b; 4a+38b; 4a+39b; 4a+40b; 4a+41b; 4a+42b; 4a+43b; 4a+44b; 4a+45b; 4a+46b; 4a+47b; 4a+48b; 4a+49b; 4a+50b; 4a+51b; 4a+52b; 4a+53b; 4a+54b; 4a+55b; 4a+55b; 4a+57b; 4a+58b; 4a+59b; 4a+60b; 4a+61b; 4a+62b; 4a+63b; 4a+64b; 4a+65b; 4a+66b; 4a+67b; 4a+68b; 4a+69b; 4a+70b; 4a+71b; 4a+72b; 4a+73b; 4a+74b; 4a+75b; 4a+76b; 4a+77b; 4a+78b; 4a+79b; 4a+80b; 4a+81b; 4a+82b; 4a+83b; 4a+84b; 4a+85b; 4a+86b; 4a+87b; 4a+88b; 4a+89b; 4a+90b; 4a+91b; 4a+92b; 4a+93b; 4a+94b; 4a+95b; 4a+96b; 4a+97b; 5a+12b; 5a+13b; 5a+14b; 5a+15b; 5a+16b; 5a+17b; 5a+18b; 5a+19b; 5a+20b; 5a+21b; 5a+22b; 5a+23b; 5a+24b; 5a+25b; 5a+26b; 5a+27b; 5a+28b; 5a+29b; 5a+30b; 5a+31b; 5a+32b; 5a+33b; 5a+34b; 5a+35b; 5a+36b; 5a+37b; 5a+38b; 5a+39b; 5a+40b; 5a+41b; 5a+42b; 5a+43b; 5a+44b; 5a+45b; 5a+46b; 5a+47b; 5a+48b; 5a+49b; 5a+50b; 5a+51b; 5a+52b; 5a+53b; 5a+54b; 5a+55b; 5a+55b; 5a+57b; 5a+58b; 5a+59b; 5a+60b; 5a+61b; 5a+62b; 5a+63b; 5a+64b; 5a+65b; 5a+66b; 5a+67b; 5a+68b; 5a+69b; 5a+70b; 5a+71b; 5a+72b; 5a+73b; 5a+74b; 5a+75b; 5a+76b; 5a+77b; 5a+78b; 5a+79b; 5a+80b; 5a+81b; 5a+82b; 5a+83b; 5a+84b; 5a+85b; 5a+86b; 5a+87b; 5a+88b; 5a+89b; 5a+90b; 5a+91b; 5a+92b; 5a+93b; 5a+94b; 5a+95b; 5a+96b; 5a+97b; 6a+1b; 6a+2b; 6a+3b; 6a+4b; 6a+5b; 6a+6b; 6a+7b; 6a+8b; 6a+9b; 6a+10b; 6a+11b; 6a+12b; 6a+13b; 6a+14b; 6a+15b; 6a+16b; 6a+17b; 6a+18b; 6a+19b; 6a+20b; 6a+21b; 6a+22b; 6a+23b; 6a+24b; 6a+25b; 6a+26b; 6a+27b; 6a+28b; 6a+29b; 6a+30b; 6a+31b; 6a+32b; 6a+33b; 6a+34b; 6a+35b; 6a+36b; 6a+37b; 6a+38b; 6a+39b; 6a+40b; 6a+41b; 6a+42b; 6a+43b; 6a+44b; 6a+45b; 6a+46b; 6a+47b; 6a+48b; 6a+49b; 6a+50b; 6a+51b; 6a+52b; 6a+53b; 6a+54b; 6a+55b; 6a+55b; 6a+57b; 6a+58b; 6a+59b; 6a+60b; 6a+61b; 6a+62b; 6a+63b; 6a+64b; 6a+65b; 6a+66b; 6a+67b; 6a+68b; 6a+69b; 6a+70b; 6a+71b; 6a+72b; 6a+73b; 6a+74b; 6a+75b; 6a+76b; 6a+77b; 6a+78b; 6a+79b; 6a+80b; 6a+81b; 6a+82b; 6a+83b; 6a+84b; 6a+85b; 6a+86b; 6a+87b; 6a+88b; 6a+89b; 6a+90b; 6a+91b; 6a+92b; 6a+93b; 6a+94b; 6a+95b; 6a+96b; 6a+97b; 7a+1b; 7a+2b; 7a+3b; 7a+4b; 7a+5b; 7a+6b; 7a+7b; 7a+8b; 7a+9b; 7a+10b; 7a+11b; 7a+12b; 7a+13b; 7a+14b; 7a+15b; 7a+16b; 7a+17b; 7a+18b; 7a+19b; 7a+20b; 7a+21b; 7a+22b; 7a+23b; 7a+24b; 7a+25b; 7a+26b; 7a+27b; 7a+28b; 7a+29b; 7a+30b; 7a+31b; 7a+32b; 7a+33b; 7a+34b; 7a+35b; 7a+36b; 7a+37b; 7a+38b; 7a+39b; 7a+40b; 7a+41b; 7a+42b; 7a+43b; 7a+44b; 7a+45b; 7a+46b; 7a+47b; 7a+48b; 7a+49b; 7a+50b; 7a+51b; 7a+52b; 7a+53b; 7a+54b; 7a+55b; 7a+55b; 7a+57b; 7a+58b; 7a+59b; 7a+60b; 7a+61b; 7a+62b; 7a+63b; 7a+64b; 7a+65b; 7a+66b; 7a+67b; 7a+68b; 7a+69b; 7a+70b; 7a+71b; 7a+72b; 7a+73b; 7a+74b; 7a+75b; 7a+76b; 7a+77b; 7a+78b; 7a+79b; 7a+80b; 7a+81b; 7a+82b; 7a+83b; 7a+84b; 7a+85b; 7a+86b; 7a+87b; 7a+88b; 7a+89b; 7a+90b; 7a+91b; 7a+92b; 7a+93b; 7a+94b; 7a+95b; 7a+96b; 7a+97b; 8a+12b; 8a+13b; 8a+14b; 8a+15b; 8a+16b; 8a+17b; 8a+18b; 8a+19b; 8a+20b; 8a+21b; 8a+22b; 8a+23b; 8a+24b; 8a+25b; 8a+26b; 8a+27b; 8a+28b; 8a+29b; 8a+30b; 8a+31b; 8a+32b; 8a+33b; 8a+34b; 8a+35b; 8a+36b; 8a+37b; 8a+38b; 8a+39b; 8a+40b; 8a+41b; 8a+42b; 8a+43b; 8a+44b; 8a+45b; 8a+46b; 8a+47b; 8a+48b; 8a+49b; 8a+50b; 8a+51b; 8a+52b; 8a+53b; 8a+54b; 8a+55b; 8a+55b; 8a+57b; 8a+58b; 8a+59b; 8a+60b; 8a+61b; 8a+62b; 8a+63b; 8a+64b; 8a+65b; 8a+66b; 8a+67b; 8a+68b; 8a+69b; 8a+70b; 8a+71b; 8a+72b; 8a+73b; 8a+74b; 8a+75b; 8a+76b; 8a+77b; 8a+78b; 8a+79b; 8a+80b; 8a+81b; 8a+82b; 8a+83b; 8a+84b; 8a+85b; 8a+86b; 8a+87b; 8a+88b; 8a+89b; 8a+90b; 8a+91b; 8a+92b; 8a+93b; 8a+94b; 8a+95b; 8a+96b; 8a+97b; 9a+1b; 9a+2b; 9a+3b; 9a+4b; 9a+5b; 9a+6b; 9a+7b; 9a+8b; 9a+9b; 9a+10b; 9a+11b; 9a+12b; 9a+13b; 9a+14b; 9a+15b; 9a+16b; 9a+17b; 9a+18b; 9a+19b; 9a+20b; 9a+21b; 9a+22b; 9a+23b; 9a+24b; 9a+25b; 9a+26b; 9a+27b; 9a+28b; 9a+29b; 9a+30b; 9a+31b; 9a+32b; 9a+33b; 9a+34b; 9a+35b; 9a+36b; 9a+37b; 9a+38b; 9a+39b; 9a+40b; 9a+41b; 9a+42b; 9a+43b; 9a+44b; 9a+45b; 9a+46b; 9a+47b; 9a+48b; 9a+49b; 9a+50b; 9a+51b; 9a+52b; 9a+53b; 9a+54b; 9a+55b; 9a+55b; 9a+57b; 9a+58b; 9a+59b; 9a+60b; 9a+61b; 9a+62b; 9a+63b; 9a+64b; 9a+65b; 9a+66b; 9a+67b; 9a+68b; 9a+69b; 9a+70b; 9a+71b; 9a+72b; 9a+73b; 9a+74b; 9a+75b; 9a+76b; 9a+77b; 9a+78b; 9a+79b; 9a+80b; 9a+81b; 9a+82b; 9a+83b; 9a+84b; 9a+85b; 9a+86b; 9a+87b; 9a+88b; 9a+89b; 9a+90b; 9a+91b; 9a+92b; 9a+93b; 9a+94b; 9a+95b; 9a+96b; 9a+97b; 10a+1b; 10a+2b; 10a+3b; 10a+4b; 10a+5b; 10a+6b; 10a+7b; 10a+8b; 10a+9b; 10a+10b; 10a+11b; 10a+12b; 10a+13b; 10a+14b; 10a+15b; 10a+16b; 10a+17b; 10a+18b; 10a+19b; 10a+20b; 10a+21b; 10a+22b; 10a+23b; 10a+24b; 10a+25b; 10a+26b; 10a+27b; 10a+28b; 10a+29b; 10a+30b; 10a+31b; 10a+32b; 10a+33b; 10a+34b; 10a+35b; 10a+36b; 10a+37b; 10a+38b; 10a+39b; 10a+40b; 10a+41b; 10a+42b; 10a+43b; 10a+44b; 10a+45b; 10a+46b; 10a+47b; 10a+48b; 10a+49b; 10a+50b; 10a+51b; 10a+52b; 10a+53b; 10a+54b; 10a+55b; 10a+55b; 10a+57b; 10a+58b; 10a+59b; 10a+60b; 10a+61b; 10a+62b; 10a+63b; 10a+64b; 10a+65b; 10a+66b; 10a+67b; 10a+68b; 10a+69b; 10a+70b; 10a+71b; 10a+72b; 10a+73b; 10a+74b; 10a+75b; 10a+76b; 10a+77b; 10a+78b; 10a+79b; 10a+80b; 10a+81b; 10a+82b; 10a+83b; 10a+84b; 10a+85b; 10a+86b; 10a+87b; 10a+88b; 10a+89b; 10a+90b; 10a+91b; 10a+92b; 10a+93b; 10a+94b; 10a+95b; 10a+96b; 10a+97b; 11a+1b; 11a+2b; 11a+3b; 11a+4b; 11a+5b; 11a+6b; 11a+7b; 11a+8b; 11a+9b; 11a+10b; 11a+11b; 11a+12b; 11a+13b; 11a+14b; 11a+15b; 11a+16b; 11a+17b; 11a+18b; 11a+19b; 11a+20b; 11a+21b; 11a+22b; 11a+23b; 11a+24b; 11a+25b; 11a+26b; 11a+27b; 11a+28b; 11a+29b; 11a+30b; 11a+31b; 11a+32b; 11a+33b; 11a+34b; 11a+35b; 11a+36b; 11a+37b; 11a+38b; 11a+39b; 11a+40b; 11a+41b; 11a+42b; 11a+43b; 11a+44b; 11a+45b; 11a+46b; 11a+47b; 11a+48b; 11a+49b; 11a+50b; 11a+51b; 11a+52b; 11a+53b; 11a+54b; 11a+55b; 11a+55b; 11a+57b; 11a+58b; 11a+59b; 11a+60b; 11a+61b; 11a+62b; 11a+63b; 11a+64b; 11a+65b; 11a+66b; 11a+67b; 11a+68b; 11a+69b; 11a+70b; 11a+71b; 11a+72b; 11a+73b; 11a+74b; 11a+75b; 11a+76b; 11a+77b; 11a+78b; 11a+79b; 11a+80b; 11a+81b; 11a+82b; 11a+83b; 11a+84b; 11a+85b; 11a+86b; 11a+87b; 11a+88b; 11a+89b; 11a+90b; 11a+91b; 11a+92b; 11a+93b; 11a+94b; 11a+95b; 11a+96b; 11a+97b; 12a+1b; 12a+2b; 12a+3b; 12a+4b; 12a+5b; 12a+6b; 12a+7b; 12a+8b; 12a+9b; 12a+10b; 12a+11b; 12a+12b; 12a+13b; 12a+14b; 12a+15b; 12a+16b; 12a+17b; 12a+18b; 12a+19b; 12a+20b; 12a+21b; 12a+22b; 12a+23b; 12a+24b; 12a+25b; 12a+26b; 12a+27b; 12a+28b; 12a+29b; 12a+30b; 12a+31b; 12a+32b; 12a+33b; 12a+34b; 12a+35b; 12a+36b; 12a+37b; 12a+38b; 12a+39b; 12a+40b; 12a+41b; 12a+42b; 12a+43b; 12a+44b; 12a+45b; 12a+46b; 12a+47b; 12a+48b; 12a+49b; 12a+50b; 12a+51b; 12a+52b; 12a+53b; 12a+54b; 12a+55b; 12a+55b; 12a+57b; 12a+58b; 12a+59b; 12a+60b; 12a+61b; 12a+62b; 12a+63b; 12a+64b; 12a+65b; 12a+66b; 12a+67b; 12a+68b; 12a+69b; 12a+70b; 12a+71b; 12a+72b; 12a+73b; 12a+74b; 12a+75b; 12a+76b; 12a+77b; 12a+78b; 12a+79b; 12a+80b; 12a+81b; 12a+82b; 12a+83b; 12a+84b; 12a+85b; 12a+86b; 12a+87b; 12a+88b; 12a+89b; 12a+90b; 12a+91b; 12a+92b; 12a+93b; 12a+94b; 12a+95b; 12a+96b; 12a+97b; 13a+1b; 13a+2b; 13a+3b; 13a+4b; 13a+5b; 13a+6b; 13a+7b; 13a+8b; 13a+9b; 13a+10b; 13a+11b; 13a+12b; 13a+13b; 13a+14b; 13a+15b; 13a+16b; 13a+17b; 13a+18b; 13a+19b; 13a+20b; 13a+21b; 13a+22b; 13a+23b; 13a+24b; 13a+25b; 13a+26b; 13a+27b; 13a+28b; 13a+29b; 13a+30b; 13a+31b; 13a+32b; 13a+33b; 13a+34b; 13a+35b; 13a+36b; 13a+37b; 13a+38b; 13a+39b; 13a+40b; 13a+41b; 13a+42b; 13a+43b; 13a+44b; 13a+45b; 13a+46b; 13a+47b; 13a+48b; 13a+49b; 13a+50b; 13a+51b; 13a+52b; 13a+53b; 13a+54b; 13a+55b; 13a+55b; 13a+57b; 13a+58b; 13a+59b; 13a+60b; 13a+61b; 13a+62b; 13a+63b; 13a+64b; 13a+65b; 13a+66b; 13a+67b; 13a+68b; 13a+69b; 13a+70b; 13a+71b; 13a+72b; 13a+73b; 13a+74b; 13a+75b; 13a+76b; 13a+77b; 13a+78b; 13a+79b; 13a+80b; 13a+81b; 13a+82b; 13a+83b; 13a+84b; 13a+85b; 13a+86b; 13a+87b; 13a+88b; 13a+89b; 13a+90b; 13a+91b; 13a+92b; 13a+93b; 13a+94b; 13a+95b; 13a+96b; 13a+97b; 14a+1b; 14a+2b; 14a+3b; 14a+4b; 14a+5b; 14a+6b; 14a+7b; 14a+8b; 14a+9b; 14a+10b; 14a+11b; 14a+12b; 14a+13b; 14a+14b; 14a+15b; 14a+16b; 14a+17b; 14a+18b; 14a+19b; 14a+20b; 14a+21b; 14a+22b; 14a+23b; 14a+24b; 14a+25b; 14a+26b; 14a+27b; 14a+28b; 14a+29b; 14a+30b; 14a+31b; 14a+32b; 14a+33b; 14a+34b; 14a+35b; 14a+36b; 14a+37b; 14a+38b; 14a+39b; 14a+40b; 14a+41b; 14a+42b; 14a+43b; 14a+44b; 14a+45b; 14a+46b; 14a+47b; 14a+48b; 14a+49b; 14a+50b; 14a+51b; 14a+52b; 14a+53b; 14a+54b; 14a+55b; 14a+55b; 14a+57b; 14a+58b; 14a+59b; 14a+60b; 14a+61b; 14a+62b; 14a+63b; 14a+64b; 14a+65b; 14a+66b; 14a+67b; 14a+68b; 14a+69b; 14a+70b; 14a+71b; 14a+72b; 14a+73b; 14a+74b; 14a+75b; 14a+76b; 14a+77b; 14a+78b; 14a+79b; 14a+80b; 14a+81b; 14a+82b; 14a+83b; 14a+84b; 14a+85b; 14a+86b; 14a+87b; 14a+88b; 14a+89b; 14a+90b; 14a+91b; 14a+92b; 14a+93b; 14a+94b; 14a+95b; 14a+96b; 14a+97b; 15a+1b; 15a+2b; 15a+3b; 15a+4b; 15a+5b; 15a+6b; 15a+7b; 15a+8b; 15a+9b; 15a+10b; 15a+11b; 15a+12b; 15a+13b; 15a+14b; 15a+15b; 15a+16b; 15a+17b; 15a+18b; 15a+19b; 15a+20b; 15a+21b; 15a+22b; 15a+23b; 15a+24b; 15a+25b; 15a+26b; 15a+27b; 15a+28b; 15a+29b; 15a+30b; 15a+31b; 15a+32b; 15a+33b; 15a+34b; 15a+35b; 15a+36b; 15a+37b; 15a+38b; 15a+39b; 15a+40b; 15a+41b; 15a+42b; 15a+43b; 15a+44b; 15a+45b; 15a+46b; 15a+47b; 15a+48b; 15a+49b; 15a+50b; 15a+51b; 15a+52b; 15a+53b; 15a+54b; 15a+55b; 15a+55b; 15a+57b; 15a+58b; 15a+59b; 15a+60b; 15a+61b; 15a+62b; 15a+63b; 15a+64b; 15a+65b; 15a+66b; 15a+67b; 15a+68b; 15a+69b; 15a+70b; 15a+71b; 15a+72b; 15a+73b; 15a+74b; 15a+75b; 15a+76b; 15a+77b; 15a+78b; 15a+79b; 15a+80b; 15a+81b; 15a+82b; 15a+83b; 15a+84b; 15a+85b; 15a+86b; 15a+87b; 15a+88b; 15a+89b; 15a+90b; 15a+91b; 15a+92b; 15a+93b; 15a+94b; 15a+95b; 15a+96b; 15a+97b; 16a+1b; 16a+2b; 16a+3b; 16a+4b; 16a+5b; 16a+6b; 16a+7b; 16a+8b; 16a+9b; 16a+10b; 16a+11b; 16a+12b; 16a+13b; 16a+14b; 16a+15b; 16a+16b; 16a+17b; 16a+18b; 16a+19b; 16a+20b; 16a+21b; 16a+22b; 16a+23b; 16a+24b; 16a+25b; 16a+26b; 16a+27b; 16a+28b; 16a+29b; 16a+30b; 16a+31b; 16a+32b; 16a+33b; 16a+34b; 16a+35b; 16a+36b; 16a+37b; 16a+38b; 16a+39b; 16a+40b; 16a+41b; 16a+42b; 16a+43b; 16a+44b; 16a+45b; 16a+46b; 16a+47b; 16a+48b; 16a+49b; 16a+50b; 16a+51b; 16a+52b; 16a+53b; 16a+54b; 16a+55b; 16a+55b; 16a+57b; 16a+58b; 16a+59b; 16a+60b; 16a+61b; 16a+62b; 16a+63b; 16a+64b; 16a+65b; 16a+66b; 16a+67b; 16a+68b; 16a+69b; 16a+70b; 16a+71b; 16a+72b; 16a+73b; 16a+74b; 16a+75b; 16a+76b; 16a+77b; 16a+78b; 16a+79b; 16a+80b; 16a+81b; 16a+82b; 16a+83b; 16a+84b; 16a+85b; 16a+86b; 16a+87b; 16a+88b; 16a+89b; 16a+90b; 16a+91b; 16a+92b; 16a+93b; 16a+94b; 16a+95b; 16a+96b; 16a+97b; 17a+1b; 17a+2b; 17a+3b; 17a+4b; 17a+5b; 17a+6b; 17a+7b; 17a+8b; 17a+9b; 17a+10b; 17a+11b; 17a+12b; 17a+13b; 17a+14b; 17a+15b; 17a+16b; 17a+17b; 17a+18b; 17a+19b; 17a+20b; 17a+21b; 17a+22b; 17a+23b; 17a+24b; 17a+25b; 17a+26b; 17a+27b; 17a+28b; 17a+29b; 17a+30b; 17a+31b; 17a+32b; 17a+33b; 17a+34b; 17a+35b; 17a+36b; 17a+37b; 17a+38b; 17a+39b; 17a+40b; 17a+41b; 17a+42b; 17a+43b; 17a+44b; 17a+45b; 17a+46b; 17a+47b; 17a+48b; 17a+49b; 17a+50b; 17a+51b; 17a+52b; 17a+53b; 17a+54b; 17a+55b; 17a+55b; 17a+57b; 17a+58b; 17a+59b; 17a+60b; 17a+61b; 17a+62b; 17a+63b; 17a+64b; 17a+65b; 17a+66b; 17a+67b; 17a+68b; 17a+69b; 17a+70b; 17a+71b; 17a+72b; 17a+73b; 17a+74b; 17a+75b; 17a+76b; 17a+77b; 17a+78b; 17a+79b; 17a+80b; 17a+81b; 17a+82b; 17a+83b; 17a+84b; 17a+85b; 17a+86b; 17a+87b; 17a+88b; 17a+89b; 17a+90b; 17a+91b; 17a+92b; 17a+93b; 17a+94b; 17a+95b; 17a+96b; 17a+97b; 18a+1b; 18a+2b; 18a+3b; 18a+4b; 18a+5b; 18a+6b; 18a+7b; 18a+8b; 18a+9b; 18a+10b; 18a+11b; 18a+12b; 18a+13b; 18a+14b; 18a+15b; 18a+16b; 18a+17b; 18a+18b; 18a+19b; 18a+20b; 18a+21b; 18a+22b; 18a+23b; 18a+24b; 18a+25b; 18a+26b; 18a+27b; 18a+28b; 18a+29b; 18a+30b; 18a+31b; 18a+32b; 18a+33b; 18a+34b; 18a+35b; 18a+36b; 18a+37b; 18a+38b; 18a+39b; 18a+40b; 18a+41b; 18a+42b; 18a+43b; 18a+44b; 18a+45b; 18a+46b; 18a+47b; 18a+48b; 18a+49b; 18a+50b; 18a+51b; 18a+52b; 18a+53b; 18a+54b; 18a+55b; 18a+55b; 18a+57b; 18a+58b; 18a+59b; 18a+60b; 18a+61b; 18a+62b; 18a+63b; 18a+64b; 18a+65b; 18a+66b; 18a+67b; 18a+68b; 18a+69b; 18a+70b; 18a+71b; 18a+72b; 18a+73b; 18a+74b; 18a+75b; 18a+76b; 18a+77b; 18a+78b; 18a+79b; 18a+80b; 18a+81b; 18a+82b; 18a+83b; 18a+84b; 18a+85b; 18a+86b; 18a+87b; 18a+88b; 18a+89b; 18a+90b; 18a+91b; 18a+92b; 18a+93b; 18a+94b; 18a+95b; 18a+96b; 18a+97b; 19a+1b; 19a+2b; 19a+3b; 19a+4b; 19a+5b; 19a+6b; 19a+7b; 19a+8b; 19a+9b; 19a+10b; 19a+11b; 19a+12b; 19a+13b; 19a+14b; 19a+15b; 19a+16b; 19a+17b; 19a+18b; 19a+19b; 19a+20b; 19a+21b; 19a+22b; 19a+23b; 19a+24b; 19a+25b; 19a+26b; 19a+27b; 19a+28b; 19a+29b; 19a+30b; 19a+31b; 19a+32b; 19a+33b; 19a+34b; 19a+35b; 19a+36b; 19a+37b; 19a+38b; 19a+39b; 19a+40b; 19a+41b; 19a+42b; 19a+43b; 19a+44b; 19a+45b; 19a+46b; 19a+47b; 19a+48b; 19a+49b; 19a+50b; 19a+51b; 19a+52b; 19a+53b; 19a+54b; 19a+55b; 19a+55b; 19a+57b; 19a+58b; l9a+59b; 19a+60b; 19a+61b; 19a+62b; 19a+63b; 19a+64b; 19a+65b; 19a+66b; 19a+67b; 19a+68b; 19a+69b; 19a+70b; 19a+71b; 19a+72b; 19a+73b; 19a+74b; 19a+75b; 19a+76b; 19a+77b; 19a+78b; 19a+79b; 19a+80b; 19a+81b; 19a+82b; 19a+83b; 19a+84b; 19a+85b; 19a+86b; 19a+87b; 19a+88b; 19a+89b; 19a+90b; 19a+91b; 19a+92b; 19a+93b; 19a+94b; 19a+95b; 19a+96b; 19a+97b; 20a+1b; 20a+2b; 20a+3b; 20a+4b; 20a+5b; 20a+6b; 20a+7b; 20a+8b; 20a+9b; 20a+10b; 20a+11b; 20a+12b; 20a+13b; 20a+14b; 20a+15b; 20a+16b; 20a+17b; 20a+18b; 20a+19b; 20a+20b; 20a+21b; 20a+22b; 20a+23b; 20a+24b; 20a+25b; 20a+26b; 20a+27b; 20a+28b; 20a+29b; 20a+30b; 20a+31b; 20a+32b; 20a+33b; 20a+34b; 20a+35b; 20a+36b; 20a+37b; 20a+38b; 20a+39b; 20a+40b; 20a+41b; 20a+42b; 20a+43b; 20a+44b; 20a+45b; 20a+46b; 20a+47b; 20a+48b; 20a+49b; 20a+50b; 20a+51b; 20a+52b; 20a+53b; 20a+54b; 20a+55b; 20a+55b; 20a+57b; 20a+58b; 20a+59b; 20a+60b; 20a+61b; 20a+62b; 20a+63b; 20a+64b; 20a+65b; 20a+66b; 20a+67b; 20a+68b; 20a+69b; 20a+70b; 20a+71b; 20a+72b; 20a+73b; 20a+74b; 20a+75b; 20a+76b; 20a+77b; 20a+78b; 20a+79b; 20a+80b; 20a+81b; 20a+82b; 20a+83b; 20a+84b; 20a+85b; 20a+86b; 20a+87b; 20a+88b; 20a+89b; 20a+90b; 20a+91b; 20a+92b; 20a+93b; 20a+94b; 20a+95b; 20a+96b; 20a+97b; 21a+1b; 21a+2b; 21a+3b; 21a+4b; 21a+5b; 21a+6b; 21a+7b; 21a+8b; 21a+9b; 21a+10b; 21a+11b; 21a+12b; 21a+13b; 21a+14b; 21a+15b; 21a+16b; 21a+17b; 21a+18b; 21a+19b; 21a+20b; 21a+21b; 21a+22b; 21a+23b; 21a+24b; 21a+25b; 21a+26b; 21a+27b; 21a+28b; 21a+29b; 21a+30b; 21a+31b; 21a+32b; 21a+33b; 21a+34b; 21a+35b; 21a+36b; 21a+37b; 21a+38b; 21a+39b; 21a+40b; 21a+41b; 21a+42b; 21a+43b; 21a+44b; 21a+45b; 21a+46b; 21a+47b; 21a+48b; 21a+49b; 21a+50b; 21a+51b; 21a+52b; 21a+53b; 21a+54b; 21a+55b; 21a+55b; 21a+57b; 21a+58b; 21a+59b; 21a+60b; 21a+61b; 21a+62b; 21a+63b; 21a+64b; 21a+65b; 21a+66b; 21a+67b; 21a+68b; 21a+69b; 21a+70b; 21a+71b; 21a+72b; 21a+73b; 21a+74b; 21a+75b; 21a+76b; 21a+77b; 21a+78b; 21a+79b; 21a+80b; 21a+81b; 21a+82b; 21a+83b; 21a+84b; 21a+85b; 21a+86b; 21a+87b; 21a+88b; 21a+89b; 21a+90b; 21a+91b; 21a+92b; 21a+93b; 21a+94b; 21a+95b; 21a+96b; 21a+97b; 22a+1b; 22a+2b; 22a+3b; 22a+4b; 22a+5b; 22a+6b; 22a+7b; 22a+8b; 22a+9b; 22a+10b; 22a+11b; 22a+12b; 22a+13b; 22a+14b; 22a+15b; 22a+16b; 22a+17b; 22a+18b; 22a+19b; 22a+20b; 22a+21b; 22a+22b; 22a+23b; 22a+24b; 22a+25b; 22a+26b; 22a+27b; 22a+28b; 22a+29b; 22a+30b; 22a+31b; 22a+32b; 22a+33b; 22a+34b; 22a+35b; 22a+36b; 22a+37b; 22a+38b; 22a+39b; 22a+40b; 22a+41b; 22a+42b; 22a+43b; 22a+44b; 22a+45b; 22a+46b; 22a+47b; 22a+48b; 22a+49b; 22a+50b; 22a+51b; 22a+52b; 22a+53b; 22a+54b; 22a+55b; 22a+55b; 22a+57b; 22a+58b; 22a+59b; 22a+60b; 22a+61b; 22a+62b; 22a+63b; 22a+64b; 22a+65b; 22a+66b; 22a+67b; 22a+68b; 22a+69b; 22a+70b; 22a+71b; 22a+72b; 22a+73b; 22a+74b; 22a+75b; 22a+76b; 22a+77b; 22a+78b; 22a+79b; 22a+80b; 22a+81b; 22a+82b; 22a+83b; 22a+84b; 22a+85b; 22a+86b; 22a+87b; 22a+88b; 22a+89b; 22a+90b; 22a+91b; 22a+92b; 22a+93b; 22a+94b; 22a+95b; 22a+96b; 22a+97b; 23a+1b; 23a+2b; 23a+3b; 23a+4b; 23a+5b; 23a+6b; 23a+7b; 23a+8b; 23a+9b; 23a+10b; 23a+11b; 23a+12b; 23a+13b; 23a+14b; 23a+15b; 23a+16b; 23a+17b; 23a+18b; 23a+19b; 23a+20b; 23a+21b; 23a+22b; 23a+23b; 23a+24b; 23a+25b; 23a+26b; 23a+27b; 23a+28b; 23a+29b; 23a+30b; 23a+31b; 23a+32b; 23a+33b; 23a+34b; 23a+35b; 23a+36b; 23a+37b; 23a+38b; 23a+39b; 23a+40b; 23a+41b; 23a+42b; 23a+43b; 23a+44b; 23a+45b; 23a+46b; 23a+47b; 23a+48b; 23a+49b; 23a+50b; 23a+51b; 23a+52b; 23a+53b; 23a+54b; 23a+55b; 23a+55b; 23a+57b; 23a+58b; 23a+59b; 23a+60b; 23a+61b; 23a+62b; 23a+63b; 23a+64b; 23a+65b; 23a+66b; 23a+67b; 23a+68b; 23a+69b; 23a+70b; 23a+71b; 23a+72b; 23a+73b; 23a+74b; 23a+75b; 23a+76b; 23a+77b; 23a+78b; 23a+79b; 23a+80b; 23a+81b; 23a+82b; 23a+83b; 23a+84b; 23a+85b; 23a+86b; 23a+87b; 23a+88b; 23a+89b; 23a+90b; 23a+91b; 23a+92b; 23a+93b; 23a+94b; 23a+95b; 23a+96b; 23a+97b; 24a+1b; 24a+2b; 24a+3b; 24a+4b; 24a+5b; 24a+6b; 24a+7b; 24a+8b; 24a+9b; 24a+10b; 24a+11b; 24a+12b; 24a+13b; 24a+14b; 24a+15b; 24a+16b; 24a+17b; 24a+18b; 24a+19b; 24a+20b; 24a+21b; 24a+22b; 24a+23b; 24a+24b; 24a+25b; 24a+26b; 24a+27b; 24a+28b; 24a+29b; 24a+30b; 24a+31b; 24a+32b; 24a+33b; 24a+34b; 24a+35b; 24a+36b; 24a+37b; 24a+38b; 24a+39b; 24a+40b; 24a+41b; 24a+42b; 24a+43b; 24a+44b; 24a+45b; 24a+46b; 24a+47b; 24a+48b; 24a+49b; 24a+50b; 24a+51b; 24a+52b; 24a+53b; 24a+54b; 24a+55b; 24a+55b; 24a+57b; 24a+58b; 24a+59b; 24a+60b; 24a+61b; 24a+62b; 24a+63b; 24a+64b; 24a+65b; 24a+66b; 24a+67b; 24a+68b; 24a+69b; 24a+70b; 24a+71b; 24a+72b; 24a+73b; 24a+74b; 24a+75b; 24a+76b; 24a+77b; 24a+78b; 24a+79b; 24a+80b; 24a+81b; 24a+82b; 24a+83b; 24a+84b; 24a+85b; 24a+86b; 24a+87b; 24a+88b; 24a+89b; 24a+90b; 24a+91b; 24a+92b; 24a+93b; 24a+94b; 24a+95b; 24a+96b; 24a+97b; 25a+1b; 25a+2b; 25a+3b; 25a+4b; 25a+5b; 25a+6b; 25a+7b; 25a+8b; 25a+9b; 25a+10b; 25a+11b; 25a+12b; 25a+13b; 25a+14b; 25a+15b; 25a+16b; 25a+17b; 25a+18b; 25a+19b; 25a+20b; 25a+21b; 25a+22b; 25a+23b; 25a+24b; 25a+25b; 25a+26b; 25a+27b; 25a+28b; 25a+29b; 25a+30b; 25a+31b; 25a+32b; 25a+33b; 25a+34b; 25a+35b; 25a+36b; 25a+37b; 25a+38b; 25a+39b; 25a+40b; 25a+41b; 25a+42b; 25a+43b; 25a+44b; 25a+45b; 25a+46b; 25a+47b; 25a+48b; 25a+49b; 25a+50b; 25a+51b; 25a+52b; 25a+53b; 25a+54b; 25a+55b; 25a+55b; 25a+57b; 25a+58b; 25a+59b; 25a+60b; 25a+61b; 25a+62b; 25a+63b; 25a+64b; 25a+65b; 25a+66b; 25a+67b; 25a+68b; 25a+69b; 25a+70b; 25a+71b; 25a+72b; 25a+73b; 25a+74b; 25a+75b; 25a+76b; 25a+77b; 25a+78b; 25a+79b; 25a+80b; 25a+81b; 25a+82b; 25a+83b; 25a+84b; 25a+85b; 25a+86b; 25a+87b; 25a+88b; 25a+89b; 25a+90b; 25a+91b; 25a+92b; 25a+93b; 25a+94b; 25a+95b; 25a+96b; 25a+97b; 26a+1b; 26a+2b; 26a+3b; 26a+4b; 26a+5b; 26a+6b; 26a+7b; 26a+8b; 26a+9b; 26a+10b; 26a+11b; 26a+12b; 26a+13b; 26a+14b; 26a+15b; 26a+16b; 26a+17b; 26a+18b; 26a+19b; 26a+20b; 26a+21b; 26a+22b; 26a+23b; 26a+24b; 26a+25b; 26a+26b; 26a+27b; 26a+28b; 26a+29b; 26a+30b; 26a+31b; 26a+32b; 26a+33b; 26a+34b; 26a+35b; 26a+36b; 26a+37b; 26a+38b; 26a+39b; 26a+40b; 26a+41b; 26a+42b; 26a+43b; 26a+44b; 26a+45b; 26a+46b; 26a+47b; 26a+48b; 26a+49b; 26a+50b; 26a+51b; 26a+52b; 26a+53b; 26a+54b; 26a+55b; 26a+55b; 26a+57b; 26a+58b; 26a+59b; 26a+60b; 26a+61b; 26a+62b; 26a+63b; 26a+64b; 26a+65b; 26a+66b; 26a+67b; 26a+68b; 26a+69b; 26a+70b; 26a+71b; 26a+72b; 26a+73b; 26a+74b; 26a+75b; 26a+76b; 26a+77b; 26a+78b; 26a+79b; 26a+80b; 26a+81b; 26a+82b; 26a+83b; 26a+84b; 26a+85b; 26a+86b; 26a+87b; 26a+88b; 26a+89b; 26a+90b; 26a+91b; 26a+92b; 26a+93b; 26a+94b; 26a+95b; 26a+96b; 26a+97b; 27a+1b; 27a+2b; 27a+3b; 27a+4b; 27a+5b; 27a+6b; 27a+7b; 27a+8b; 27a+9b; 27a+10b; 27a+11b; 27a+12b; 27a+13b; 27a+14b; 27a+15b; 27a+16b; 27a+17b; 27a+18b; 27a+19b; 27a+20b; 27a+21b; 27a+22b; 27a+23b; 27a+24b; 27a+25b; 27a+26b; 27a+27b; 27a+28b; 27a+29b; 27a+30b; 27a+31b; 27a+32b; 27a+33b; 27a+34b; 27a+35b; 27a+36b; 27a+37b; 27a+38b; 27a+39b; 27a+40b; 27a+41b; 27a+42b; 27a+43b; 27a+44b; 27a+45b; 27a+46b; 27a+47b; 27a+48b; 27a+49b; 27a+50b; 27a+51b; 27a+52b; 27a+53b; 27a+54b; 27a+55b; 27a+55b; 27a+57b; 27a+58b; 27a+59b; 27a+60b; 27a+61b; 27a+62b; 27a+63b; 27a+64b; 27a+65b; 27a+66b; 27a+67b; 27a+68b; 27a+69b; 27a+70b; 27a+71b; 27a+72b; 27a+73b; 27a+74b; 27a+75b; 27a+76b; 27a+77b; 27a+78b; 27a+79b; 27a+80b; 27a+81b; 27a+82b; 27a+83b; 27a+84b; 27a+85b; 27a+86b; 27a+87b; 27a+88b; 27a+89b; 27a+90b; 27a+91b; 27a+92b; 27a+93b; 27a+94b; 27a+95b; 27a+96b; 27a+97b; 28a+1b; 28a+2b; 28a+3b; 28a+4b; 28a+5b; 28a+6b; 28a+7b; 28a+8b; 28a+9b; 28a+10b; 28a+11b; 28a+12b; 28a+13b; 28a+14b; 28a+15b; 28a+16b; 28a+17b; 28a+18b; 28a+19b; 28a+20b; 28a+21b; 28a+22b; 28a+23b; 28a+24b; 28a+25b; 28a+26b; 28a+27b; 28a+28b; 28a+29b; 28a+30b; 28a+31b; 28a+32b; 28a+33b; 28a+34b; 28a+35b; 28a+36b; 28a+37b; 28a+38b; 28a+39b; 28a+40b; 28a+41b; 28a+42b; 28a+43b; 28a+44b; 28a+45b; 28a+46b; 28a+47b; 28a+48b; 28a+49b; 28a+50b; 28a+51b; 28a+52b; 28a+53b; 28a+54b; 28a+55b; 28a+55b; 28a+57b; 28a+58b; 28a+59b; 28a+60b; 28a+61b; 28a+62b; 28a+63b; 28a+64b; 28a+65b; 28a+66b; 28a+67b; 28a+68b; 28a+69b; 28a+70b; 28a+71b; 28a+72b; 28a+73b; 28a+74b; 28a+75b; 28a+76b; 28a+77b; 28a+78b; 28a+79b; 28a+80b; 28a+81b; 28a+82b; 28a+83b; 28a+84b; 28a+85b; 28a+86b; 28a+87b; 28a+88b; 28a+89b; 28a+90b; 28a+91b; 28a+92b; 28a+93b; 28a+94b; 28a+95b; 28a+96b; 28a+97b; 29a+1b; 29a+2b; 29a+3b; 29a+4b; 29a+5b; 29a+6b; 29a+7b; 29a+8b; 29a+9b; 29a+10b; 29a+11b; 29a+12b; 29a+13b; 29a+14b; 29a+15b; 29a+16b; 29a+17b; 29a+18b; 29a+19b; 29a+20b; 29a+21b; 29a+22b; 29a+23b; 29a+24b; 29a+25b; 29a+26b; 29a+27b; 29a+28b; 29a+29b; 29a+30b; 29a+31b; 29a+32b; 29a+33b; 29a+34b; 29a+35b; 29a+36b; 29a+37b; 29a+38b; 29a+39b; 29a+40b; 29a+41b; 29a+42b; 29a+43b; 29a+44b; 29a+45b; 29a+46b; 29a+47b; 29a+48b; 29a+49b; 29a+50b; 29a+51b; 29a+52b; 29a+53b; 29a+54b; 29a+55b; 29a+55b; 29a+57b; 29a+58b; 29a+59b; 29a+60b; 29a+61b; 29a+62b; 29a+63b; 29a+64b; 29a+65b; 29a+66b; 29a+67b; 29a+68b; 29a+69b; 29a+70b; 29a+71b; 29a+72b; 29a+73b; 29a+74b; 29a+75b; 29a+76b; 29a+77b; 29a+78b; 29a+79b; 29a+80b; 29a+81b; 29a+82b; 29a+83b; 29a+84b; 29a+85b; 29a+86b; 29a+87b; 29a+88b; 29a+89b; 29a+90b; 29a+91b; 29a+92b; 29a+93b; 29a+94b; 29a+95b; 29a+96b; 29a+97b; 30a+1b; 30a+2b; 30a+3b; 30a+4b; 30a+5b; 30a+6b; 30a+7b; 30a+8b; 30a+9b; 30a+10b; 30a+11b; 30a+12b; 30a+13b; 30a+14b; 30a+15b; 30a+16b; 30a+17b; 30a+18b; 30a+19b; 30a+20b; 30a+21b; 30a+22b; 30a+23b; 30a+24b; 30a+25b; 30a+26b; 30a+27b; 30a+28b; 30a+29b; 30a+30b; 30a+31b; 30a+32b; 30a+33b; 30a+34b; 30a+35b; 30a+36b; 30a+37b; 30a+38b; 30a+39b; 30a+40b; 30a+41b; 30a+42b; 30a+43b; 30a+44b; 30a+45b; 30a+46b; 30a+47b; 30a+48b; 30a+49b; 30a+50b; 30a+51b; 30a+52b; 30a+53b; 30a+54b; 30a+55b; 30a+55b; 30a+57b; 30a+58b; 30a+59b; 30a+60b; 30a+61b; 30a+62b; 30a+63b; 30a+64b; 30a+65b; 30a+66b; 30a+67b; 30a+68b; 30a+69b; 30a+70b; 30a+71b; 30a+72b; 30a+73b; 30a+74b; 30a+75b; 30a+76b; 30a+77b; 30a+78b; 30a+79b; 30a+80b; 30a+81b; 30a+82b; 30a+83b; 30a+84b; 30a+85b; 30a+86b; 30a+87b; 30a+88b; 30a+89b; 30a+90b; 30a+91b; 30a+92b; 30a+93b; 30a+94b; 30a+95b; 30a+96b; 30a+97b; 31a+1b; 31a+2b; 31a+3b; 31a+4b; 31a+5b; 31a+6b; 31a+7b; 31a+8b; 31a+9b; 31a+10b; 31a+11b; 31a+12b; 31a+13b; 31a+14b; 31a+15b; 31a+16b; 31a+17b; 31a+18b; 31a+19b; 31a+20b; 31a+21b; 31a+22b; 31a+23b; 31a+24b; 31a+25b; 31a+26b; 31a+27b; 31a+28b; 31a+29b; 31a+30b; 31a+31b; 31a+32b; 31a+33b; 31a+34b; 31a+35b; 31a+36b; 31a+37b; 31a+38b; 31a+39b; 31a+40b; 31a+41b; 31a+42b; 31a+43b; 31a+44b; 31a+45b; 31a+46b; 31a+47b; 31a+48b; 31a+49b; 31a+50b; 31a+51b; 31a+52b; 31a+53b; 31a+54b; 31a+55b; 31a+55b; 31a+57b; 31a+58b; 31a+59b; 31a+60b; 31a+61b; 31a+62b; 31a+63b; 31a+64b; 31a+65b; 31a+66b; 31a+67b; 31a+68b; 31a+69b; 31a+70b; 31a+71b; 31a+72b; 31a+73b; 31a+74b; 31a+75b; 31a+76b; 31a+77b; 31a+78b; 31a+79b; 31a+80b; 31a+81b; 31a+82b; 31a+83b; 31a+84b; 31a+85b; 31a+86b; 31a+87b; 31a+88b; 31a+89b; 31a+90b; 31a+91b; 31a+92b; 31a+93b; 31a+94b; 31a+95b; 31a+96b; 31a+97b; 32a+1b; 32a+2b; 32a+3b; 32a+4b; 32a+5b; 32a+6b; 32a+7b; 32a+8b; 32a+9b; 32a+10b; 32a+11b; 32a+12b; 32a+13b; 32a+14b; 32a+15b; 32a+16b; 32a+17b; 32a+18b; 32a+19b; 32a+20b; 32a+21b; 32a+22b; 32a+23b; 32a+24b; 32a+25b; 32a+26b; 32a+27b; 32a+28b; 32a+29b; 32a+30b; 32a+31b; 32a+32b; 32a+33b; 32a+34b; 32a+35b; 32a+36b; 32a+37b; 32a+38b; 32a+39b; 32a+40b; 32a+41b; 32a+42b; 32a+43b; 32a+44b; 32a+45b; 32a+46b; 32a+47b; 32a+48b; 32a+49b; 32a+50b; 32a+51b; 32a+52b; 32a+53b; 32a+54b; 32a+55b; 32a+55b; 32a+57b; 32a+58b; 32a+59b; 32a+60b; 32a+61b; 32a+62b; 32a+63b; 32a+64b; 32a+65b; 32a+66b; 32a+67b; 32a+68b; 32a+69b; 32a+70b; 32a+71b; 32a+72b; 32a+73b; 32a+74b; 32a+75b; 32a+76b; 32a+77b; 32a+78b; 32a+79b; 32a+80b; 32a+81b; 32a+82b; 32a+83b; 32a+84b; 32a+85b; 32a+86b; 32a+87b; 32a+88b; 32a+89b; 32a+90b; 32a+91b; 32a+92b; 32a+93b; 32a+94b; 32a+95b; 32a+96b; 32a+97b; 33a+1b; 33a+2b; 33a+3b; 33a+4b; 33a+5b; 33a+6b; 33a+7b; 33a+8b; 33a+9b; 33a+10b; 33a+11b; 33a+12b; 33a+13b; 33a+14b; 33a+15b; 33a+16b; 33a+17b; 33a+18b; 33a+19b; 33a+20b; 33a+21b; 33a+22b; 33a+23b; 33a+24b; 33a+25b; 33a+26b; 33a+27b; 33a+28b; 33a+29b; 33a+30b; 33a+31b; 33a+32b; 33a+33b; 33a+34b; 33a+35b; 33a+36b; 33a+37b; 33a+38b; 33a+39b; 33a+40b; 33a+41b; 33a+42b; 33a+43b; 33a+44b; 33a+45b; 33a+46b; 33a+47b; 33a+48b; 33a+49b; 33a+50b; 33a+51b; 33a+52b; 33a+53b; 33a+54b; 33a+55b; 33a+55b; 33a+57b; 33a+58b; 33a+59b; 33a+60b; 33a+61b; 33a+62b; 33a+63b; 33a+64b; 33a+65b; 33a+66b; 33a+67b; 33a+68b; 33a+69b; 33a+70b; 33a+71b; 33a+72b; 33a+73b; 33a+74b; 33a+75b; 33a+76b; 33a+77b; 33a+78b; 33a+79b; 33a+80b; 33a+81b; 33a+82b; 33a+83b; 33a+84b; 33a+85b; 33a+86b; 33a+87b; 33a+88b; 33a+89b; 33a+90b; 33a+91b; 33a+92b; 33a+93b; 33a+94b; 33a+95b; 33a+96b; 33a+97b; 34a+1b; 34a+2b; 34a+3b; 34a+4b; 34a+5b; 34a+6b; 34a+7b; 34a+8b; 34a+9b; 34a+10b; 34a+11b; 34a+12b; 34a+13b; 34a+14b; 34a+15b; 34a+16b; 34a+17b; 34a+18b; 34a+19b; 34a+20b; 34a+21b; 34a+22b; 34a+23b; 34a+24b; 34a+25b; 34a+26b; 34a+27b; 34a+28b; 34a+29b; 34a+30b; 34a+31b; 34a+32b; 34a+33b; 34a+34b; 34a+35b; 34a+36b; 34a+37b; 34a+38b; 34a+39b; 34a+40b; 34a+41b; 34a+42b; 34a+43b; 34a+44b; 34a+45b; 34a+46b; 34a+47b; 34a+48b; 34a+49b; 34a+50b; 34a+51b; 34a+52b; 34a+53b; 34a+54b; 34a+55b; 34a+55b; 34a+57b; 34a+58b; 34a+59b; 34a+60b; 34a+61b; 34a+62b; 34a+63b; 34a+64b; 34a+65b; 34a+66b; 34a+67b; 34a+68b; 34a+69b; 34a+70b; 34a+71b; 34a+72b; 34a+73b; 34a+74b; 34a+75b; 34a+76b; 34a+77b; 34a+78b; 34a+79b; 34a+80b; 34a+81b; 34a+82b; 34a+83b; 34a+84b; 34a+85b; 34a+86b; 34a+87b; 34a+88b; 34a+89b; 34a+90b; 34a+91b; 34a+92b; 34a+93b; 34a+94b; 34a+95b; 34a+96b; 34a+97b; 35a+1b; 35a+2b; 35a+3b; 35a+4b; 35a+5b; 35a+6b; 35a+7b; 35a+8b; 35a+9b; 35a+10b; 35a+11b; 35a+12b; 35a+13b; 35a+14b; 35a+15b; 35a+16b; 35a+17b; 35a+18b; 35a+19b; 35a+20b; 35a+21b; 35a+22b; 35a+23b; 35a+24b; 35a+25b; 35a+26b; 35a+27b; 35a+28b; 35a+29b; 35a+30b; 35a+31b; 35a+32b; 35a+33b; 35a+34b; 35a+35b; 35a+36b; 35a+37b; 35a+38b; 35a+39b; 35a+40b; 35a+41b; 35a+42b; 35a+43b; 35a+44b; 35a+45b; 35a+46b; 35a+47b; 35a+48b; 35a+49b; 35a+50b; 35a+51b; 35a+52b; 35a+53b; 35a+54b; 35a+55b; 35a+55b; 35a+57b; 35a+58b; 35a+59b; 35a+60b; 35a+61b; 35a+62b; 35a+63b; 35a+64b; 35a+65b; 35a+66b; 35a+67b; 35a+68b; 35a+69b; 35a+70b; 35a+71b; 35a+72b; 35a+73b; 35a+74b; 35a+75b; 35a+76b; 35a+77b; 35a+78b; 35a+79b; 35a+80b; 35a+81b; 35a+82b; 35a+83b; 35a+84b; 35a+85b; 35a+86b; 35a+87b; 35a+88b; 35a+89b; 35a+90b; 35a+91b; 35a+92b; 35a+93b; 35a+94b; 35a+95b; 35a+96b; 35a+97b; 36a+1b; 36a+2b; 36a+3b; 36a+4b; 36a+5b; 36a+6b; 36a+7b; 36a+8b; 36a+9b; 36a+10b; 36a+11b; 36a+12b; 36a+13b; 36a+14b; 36a+15b; 36a+16b; 36a+17b; 36a+18b; 36a+19b; 36a+20b; 36a+21b; 36a+22b; 36a+23b; 36a+24b; 36a+25b; 36a+26b; 36a+27b; 36a+28b; 36a+29b; 36a+30b; 36a+31b; 36a+32b; 36a+33b; 36a+34b; 36a+35b; 36a+36b; 36a+37b; 36a+38b; 36a+39b; 36a+40b; 36a+41b; 36a+42b; 36a+43b; 36a+44b; 36a+45b; 36a+46b; 36a+47b; 36a+48b; 36a+49b; 36a+50b; 36a+51b; 36a+52b; 36a+53b; 36a+54b; 36a+55b; 36a+55b; 36a+57b; 36a+58b; 36a+59b; 36a+60b; 36a+61b; 36a+62b; 36a+63b; 36a+64b; 36a+65b; 36a+66b; 36a+67b; 36a+68b; 36a+69b; 36a+70b; 36a+71b; 36a+72b; 36a+73b; 36a+74b; 36a+75b; 36a+76b; 36a+77b; 36a+78b; 36a+79b; 36a+80b; 36a+81b; 36a+82b; 36a+83b; 36a+84b; 36a+85b; 36a+86b; 36a+87b; 36a+88b; 36a+89b; 36a+90b; 36a+91b; 36a+92b; 36a+93b; 36a+94b; 36a+95b; 36a+96b; 36a+97b; 37a+1b; 37a+2b; 37a+3b; 37a+4b; 37a+5b; 37a+6b; 37a+7b; 37a+8b; 37a+9b; 37a+10b; 37a+11b; 37a+12b; 37a+13b; 37a+14b; 37a+15b; 37a+16b; 37a+17b; 37a+18b; 37a+19b; 37a+20b; 37a+21b; 37a+22b; 37a+23b; 37a+24b; 37a+25b; 37a+26b; 37a+27b; 37a+28b; 37a+29b; 37a+30b; 37a+31b; 37a+32b; 37a+33b; 37a+34b; 37a+35b; 37a+36b; 37a+37b; 37a+38b; 37a+39b; 37a+40b; 37a+41b;.37a+42b; 37a+43b; 37a+44b; 37a+45b; 37a+46b; 37a+47b; 37a+48b; 37a+49b; 37a+50b; 37a+51b; 37a+52b; 37a+53b; 37a+54b; 37a+55b; 37a+55b; 37a+57b; 37a+58b; 37a+59b; 37a+60b; 37a+61b; 37a+62b; 37a+63b; 37a+64b; 37a+65b; 37a+66b; 37a+67b; 37a+68b; 37a+69b; 37a+70b; 37a+71b; 37a+72b; 37a+73b; 37a+74b; 37a+75b; 37a+76b; 37a+77b; 37a+78b; 37a+79b; 37a+80b; 37a+81b; 37a+82b; 37a+83b; 37a+84b; 37a+85b; 37a+86b; 37a+87b; 37a+88b; 37a+89b; 37a+90b; 37a+91b; 37a+92b; 37a+93b; 37a+94b; 37a+95b; 37a+96b; 37a+97b; 38a+1b; 38a+2b; 38a+3b; 38a+4b; 38a+5b; 38a+6b; 38a+7b; 38a+8b; 38a+9b; 38a+10b; 38a+11b; 38a+12b; 38a+13b; 38a+14b; 38a+15b; 38a+16b; 38a+17b; 38a+18b; 38a+19b; 38a+20b; 38a+21b; 38a+22b; 38a+23b; 38a+24b; 38a+25b; 38a+26b; 38a+27b; 38a+28b; 38a+29b; 38a+30b; 38a+31b; 38a+32b; 38a+33b; 38a+34b; 38a+35b; 38a+36b; 38a+37b; 38a+38b; 38a+39b; 38a+40b; 38a+41b; 38a+42b; 38a+43b; 38a+44b; 38a+45b; 38a+46b; 38a+47b; 38a+48b; 38a+49b; 38a+50b; 38a+51b; 38a+52b; 38a+53b; 38a+54b; 38a+55b; 38a+55b; 38a+57b; 38a+58b; 38a+59b; 38a+60b; 38a+61b; 38a+62b; 38a+63b; 38a+64b; 38a+65b; 38a+66b; 38a+67b; 38a+68b; 38a+69b; 38a+70b; 38a+71b; 38a+72b; 38a+73b; 38a+74b; 38a+75b; 38a+76b; 38a+77b; 38a+78b; 38a+79b; 38a+80b; 38a+81b; 38a+82b; 38a+83b; 38a+84b; 38a+85b; 38a+86b; 38a+87b; 38a+88b; 38a+89b; 38a+90b; 38a+91b; 38a+92b; 38a+93b; 38a+94b; 38a+95b; 38a+96b; 38a+97b; 39a+1b; 39a+2b; 39a+3b; 39a+4b; 39a+5b; 39a+6b; 39a+7b; 39a+8b; 39a+9b; 39a+10b; 39a+11b; 39a+12b; 39a+13b; 39a+14b; 39a+15b; 39a+16b; 39a+17b; 39a+18b; 39a+19b; 39a+20b; 39a+21b; 39a+22b; 39a+23b; 39a+24b; 39a+25b; 39a+26b; 39a+27b; 39a+28b; 39a+29b; 39a+30b; 39a+31b; 39a+32b; 39a+33b; 39a+34b; 39a+35b; 39a+36b; 39a+37b; 39a+38b; 39a+39b; 39a+40b; 39a+41b; 39a+42b; 39a+43b; 39a+44b; 39a+45b; 39a+46b; 39a+47b; 39a+48b; 39a+49b; 39a+50b; 39a+51b; 39a+52b; 39a+53b; 39a+54b; 39a+55b; 39a+55b; 39a+57b; 39a+58b; 39a+59b; 39a+60b; 39a+61b; 39a+62b; 39a+63b; 39a+64b; 39a+65b; 39a+66b; 39a+67b; 39a+68b; 39a+69b; 39a+70b; 39a+71b; 39a+72b; 39a+73b; 39a+74b; 39a+75b; 39a+76b; 39a+77b; 39a+78b; 39a+79b; 39a+80b; 39a+81b; 39a+82b; 39a+83b; 39a+84b; 39a+85b; 39a+86b; 39a+87b; 39a+88b; 39a+89b; 39a+90b; 39a+91b; 39a+92b; 39a+93b; 39a+94b; 39a+95b; 39a+96b; and 39a+97b;

Compositions that Comprise Additional Therapeutic Agents Other Than Anti-Infective Agents

In addition to incorporation of the above-described anti-infective agents into drug combinations, one or more other pharmaceutically active agents can be incorporated into the present compositions to improve or enhance efficacy. In certain embodiments, the composition may further include a compound which acts to have an inhibitory effect on pathological processes in or around the treatment site. Representative examples of additional therapeutically active agents include, by way of example and not limitation, anti-thrombotic agents, anti-proliferative agents, anti-inflammatory agents, analgesics, neoplastic agents, enzymes, receptor antagonists or agonists, hormones, antibiotics, antimicrobial agents, antibodies, cytokine inhibitors, IMPDH (inosine monophosphate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

In certain embodiments, the composition may further include an anti-thrombotic agent and/or antiplatelet agent and/or a thrombolytic agent, which reduces the likelihood of thrombotic events upon implantation of a medical implant. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulfate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX⁹⁰⁶⁵a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

The therapeutic compositions may further include an agent from one of the following classes of compounds: anti-inflammatory agents (e.g., dexamethasone, cortisone, fludrocortisone, prednisone, prednisolone, 6α-methylprednisolone, triamcinolone, betamethasone, and aspirin); MMP inhibitors (e.g., batimistat, marimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat., Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071A1, WO 99/001545A1, WO 97/40028A1, WO 97/41211A1, WO 98/40381A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376,527; 6,444,696; 6,479,507; 6,509,361; 6,579,874, and 6,630,485, and U.S. Patent Application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2,WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, including tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of biologically active agents which may be included in the compositions of the invention include tyrosine kinase inhibitors, such as imantinib, ZK-222584, CGP-52411, CGP-53716, NVP-AAK980-NX, CP-127374, CP-564959, PD-171026, PD-173956, PD-180970, SU-0879, and SKI-606; MMP inhibitors such as nimesulide, PKF-241-466, PKF-242-484, CGS-27023A, SAR-943, primomastat, SC-77964, PNU-171829, AG-3433, PNU-142769, SU-5402, and Dexlipotam; p38 MAP kinase inhibitors such as CGH-2466 and PD-98-59; immunosuppressants such as argyrin B, macrocyclic lactone, ADZ-62-826, CC_{1- 779}, tilomisole, amcinonide, FK-778, AVE-1726, and MDL-28842; cytokine inhibitors such as TNF-484A, PD-172084, CP-293121, CP-353164, and PD-168787; NFKB inhibitors, such as, AVE-0547, AVE-0545, and IPL-576092; HMGCoA reductase inhibitors, such as, pravestatin, atorvastatin, fluvastatin, dalvastatin, glenvastatin, pitavastatin, CP-83101, U-20685; apoptosis antagonist (e.g., troloxamine, TCH-346 (N-methyl-N-propargyl-10-aminomethyl-dibenzo(b,f)oxepin); and caspase inhibitors (e.g., PF-5901 (benzenemethanol, alpha-pentyl-3-(2-quinolinylmethoxy)—), and JNK inhibitors (e.g., AS-602801).

In certain embodiments, the composition may further include an antibiotic (e.g., amoxicillin, trimethoprim-sulfamethoxazole, azithromycin, clarithromycin, amoxicillin-clavulanate, cefprozil, cefuroxime, cefpodoxime, or cefdinir) and/or an anti-fungal agent.

In certain embodiments, a composition comprising a fibrosis-inhibiting drug combination (or individual component(s) thereof) is combined with an agent that can modify metabolism of the agent in vivo to enhance efficacy of the fibrosis-inhibiting drug combination (or individual component(s) thereof). One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting drug combination (or individual component(s) thereof) and a CYP inhibitor, which may be combined (e.g., coated) with any of the devices described herein, including, without limitation, stents, grafts, patches, valves, wraps, and films. Representative examples of CYP inhibitors include flavones, azole antifungals, macrolide antibiotics, HIV protease inhibitors, and anti-sense oligomers. Devices comprising a combination of a fibrosis-inhibiting drug combination and a CYP inhibitor may be used to treat a variety of proliferative conditions that can lead to undesired scarring of tissue, including intimal hyperplasia, surgical adhesions, and tumor growth.

In certain embodiments, the anti-fibrosis drug combination (or individual component(s) thereof) may be further combined with anti-thrombotic and/or antiplatelet agents (for example, heparin, dextran sulfate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

Compositions That Comprise Additional Components

Within certain embodiments of the invention, the composition can also comprise radio-opaque, echogenic materials and magnetic resonance imaging (MRI) responsive materials (i.e., MRI contrast agents) to aid in visualization of the composition under ultrasound, fluoroscopy and/or MRI. For example, a composition may be echogenic or radiopaque (e.g., made with echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). For visualization under MRI, contrast agents (e.g., gadolinium (III) chelates or iron oxide compounds) may be incorporated into the composition.

The compositions may, alternatively, or in addition, be visualized under visible light, using fluorescence, or by other spectroscopic means. Visualization agents that can be included for this purpose include dyes, pigments, and other colored agents. In certain embodiments, the composition may further include a colorant to improve visualization of the composition in vivo and/or ex vivo. Frequently, compositions can be difficult to visualize upon delivery into a host, especially at the margins of an implant or tissue. A coloring agent can be incorporated into a composition to reduce or eliminate the incidence or severity of this problem. The coloring agent provides a unique color, increased contrast, or unique fluorescence characteristics to the composition. In certain embodiments, a composition is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan.

In certain embodiments, the compositions of the present invention include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In certain embodiments, the compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In certain embodiments, the compositions of the present invention include one or more bactericidal (also known as bacteriacidal) agents.

In certain embodiments, the compositions of the present invention include one or more antioxidants, present in an effective amount. Examples of the antioxidant include sulfites, alpha-tocopherol, beta-carotene and ascorbic acid.

Although the above therapeutic agents (e.g., individual components of anti-scarring drug combinations, secondary agents, other components in the compositions according to the present invention) have been provided for the purposes of illustration, it should be understood that the present invention is not so limited. For example, although agents are specifically referred to above, the present invention should be understood to include analogues, derivatives, pharmaceutically active or acceptable salts, metabolites, and conjugates of such agents. In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially. However, for the individual components of anti-scarring combinations, if they are delivered sequentially, there have to be a time during which they are all present as a composition after delivery.

Characteristics of Certain Compositions

In certain embodiments, compositions of the present invention may have a stable shelf-life of at least several months and capable of being produced and maintained under sterile conditions. The composition may be sterile either by preparing them under aseptic environment and/or they may be terminally sterilized using methods known in the art. A combination of both of these methods may also be used to prepare the composition in the sterile form. Sterilization may also occur by terminally using gamma radiation or electron beam sterilization methods.

In certain embodiments, the compositions of the present invention are sterile. Many pharmaceuticals are manufactured to be sterile and this criterion is defined by the USP XXII<1211>. The term “USP” refers to U.S. Pharmacopeia (see www.usp.org, Rockville, Md.). Sterilization in this embodiment may be accomplished by a number of means accepted in the industry and listed in the USP XXII<1211>, including gas sterilization, ionizing radiation or, when appropriate, filtration. Sterilization may be maintained by what is termed asceptic processing, defined also in USP XXII<1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E. I. DuPont De Nemours and Company, Wilmington, Del.).

In another aspect, the compositions of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII<661>), an acceptable limit of extractables within the container material (refer to USP XXII), an acceptable barrier capacity for moisture (refer to USP XXII<671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII<661>), polyethylene, TEFLON, silicone, and gray-butyl rubber. For parenterals, USP Types I to III glass and polyethylene are preferred.

Within certain aspects of the present invention, the therapeutic composition should be biocompatible, and release one or more fibrosis-inhibiting agents over a period of several hours, days, or, months. As described above, “release of an agent” refers to any statistically significant presence of the agent, or a subcomponent thereof, which has dissociated from the compositions. The compositions of the present invention may release anti-scarring agent(s) at one or more phases, the one or more phases having similar or different performance (e.g., release) profiles. The therapeutic agent(s) may be made available to the tissue at amounts which may be sustainable, intermittent, or continuous; in one or more phases; and/or rates of delivery; effective to reduce or inhibit any one or more components of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing an anti-scarring drug combination (or individual component(s) thereof) at a time such that at least one of the components of fibrosis is inhibited or reduced. Moreover, the predetermined release rate may reduce agent loading and/or concentration as well as potentially providing minimal drug washout and thus, increases efficiency of drug effect. The anti-scarring drug combination (or individual component(s) thereof) may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring drug combination (or individual component(s) thereof) to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, substantially non-releasing.

The total amount of anti-scarring drug combination (or individual component(s) thereof) made available on, in or near the device may be in an amount ranging from about 0.01 μg (micrograms) to about 2500 mg (milligrams). Generally, the anti-scarring drug combination (or individual component(s) thereof) may be in the amount ranging from 0.01 μg to about 10 μg; or from 10 μg to about 1 mg; or from 1 mg to about mg; or from 10 mg to about 100 mg; or from 100 mg to about 500 mg; or from 500 mg to about 2500 mg.

The total surface amount of anti-scarring drug combination (or individual component(s) thereof) on, in or near the device may be in an amount ranging from less than 0.01 μg to about 2500 μg per mm² of device surface area. Generally, the anti-scarring drug combination (or individual component(s) thereof) may be in the amount ranging from less than 0.01 μg; or from 0.01 μg to about 10 μg; or from 10 μg to about 250 μg; or from 250 μg to about 2500 μg.

The anti-scarring drug combination (or individual component(s) thereof) that is on, in or near the device may be released from the composition in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The amount of anti-scarring drug combination (or individual component(s) thereof) released from the composition as a function of time may be determined based on the in vitro release characteristics of the agent from the composition. The in vitro release rate may be determined by placing the anti-scarring drug combination (or individual component(s) thereof) within the composition or device in an appropriate buffer such as 0.1M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring drug combination (or individual component(s) thereof) per day may range from an amount ranging from about 0.01 μg (micrograms) to about 2500 mg (milligrams). Generally, the anti-scarring drug combination (or individual component(s) thereof) that may be released in a day may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the anti-scarring drug combination (or individual component(s) thereof) is made available to the susceptible tissue site in a programmed, sustained, and/or controlled manner which results in increased efficiency and/or efficacy. Further, the release rates may vary during either or both of the initial and subsequent release phases. There may also be additional phase(s) for release of the same substance(s) and/or different substance(s).

Delivery of Drug Combinations or Individual Components Thereof

The present invention provides various compositions that can be used to inhibit fibrosis of tissue in the vicinity of a treatment site (e.g., a surgical site). Within various embodiments, fibrosis is inhibited by local or systemic release of specific pharmacological agents that become localized at the site of intervention. Within other embodiments, fibrosis can be inhibited by local or systemic release of specific pharmacological agents that become localized adjacent to a device or implant that has been introduced into a host. In certain embodiments, compositions are provided which inhibit fibrosis in and around an implanted device, or prevent “stenosis” of a device/implant in situ, thus enhancing the efficacy.

Individual components of drug combinations may be delivered to a site of treatment together or separately. For instance, in certain embodiments, individual components are combined to form drug combinations before being delivered to a site of treatment. In certain other embodiments, individual components are delivered separately to a site of treatment and combine in situ to become drug combinations. In such embodiments, individual components may be delivered sequentially via a same delivery method (e.g., infiltrating tissue surrounding an implant or device that will be, or is, or has been, implanted), or via different delivery methods (e.g., infiltrating tissue surrounding an implant or device that will be, or is, or has been, implanted with one component, where the device is coated or otherwise combined with another component).

There are numerous methods available for optimizing delivery of anti-fibrosis drug combinations or individual components thereof to the site of the intervention. Several of these are described below.

Systemic, Regional and Local Delivery of Drug Combinations or Individual Components Thereof

A variety of drug-delivery technologies are available for systemic, regional and local delivery of anti-fibrosis drug combinations.

For systemic delivery of therapeutic agents (e.g., anti-fibrosis drug combinations or individual components thereof), several routes of administration would be suitable to provide systemic exposure of the therapeutic agent, including: (a) intravenous, (b) oral, (c) subcutaneous, (d) intraperitoneal, (e) intrathecal, (f) inhaled and intranasal, (g) sublingual or transbuccal, (h) rectal, (i) intravaginal, (j) intra-arterial, (k) intracardiac, (l) transdermal, (m) intra-ocular and (n) intramuscular. The therapeutic agents may be administered as a sustained low dose therapy to prevent disease progression, prolong disease remission, or decrease symptoms in active disease. Alternatively, the therapeutic agents may be administered in higher doses as a “pulse” therapy to induce remission in acutely active disease. The minimum dose capable of achieving these endpoints can be used and can vary according to patient, severity of disease, formulation of the administered agent, potency and tolerability of the therapeutic agent, and route of administration.

For regional and local delivery of therapeutic agents (e.g., anti-fibrosis drug combinations or individual components thereof), several techniques would be suitable to achieve preferentially elevated levels of therapeutic agents in the vicinity of the area to be treated. These include: (a) using drug-delivery catheters and/or a syringe and needle for local, regional or systemic delivery of fibrosis-inhibiting agents to the tissue surrounding the device or implant (typically, drug delivery catheters are advanced through the circulation or inserted directly into tissues under radiological guidance until they reach the desired anatomical location; the fibrosis-inhibiting agent can then be released from the catheter lumen in high local concentrations in order to deliver therapeutic doses of the drug to the tissue surrounding the device or implant); (b) drug localization techniques such as magnetic, ultrasonic or MRI-guided drug delivery; (c) chemical modification of the therapeutic drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the therapeutic drug or formulation designed to localize the drug to areas of bleeding or disrupted vasculature; and/or (e) direct injection, for example subcutaneous, intramuscular, intra-articular, etc, of the therapeutic agent, for example, under normal or endoscopic vision.

In certain embodiments, individual components of drug combinations are combined together before being systemically, regionally, or locally delivered. In certain other embodiments, individual components of drug combinations are separately delivered via a same or different systemic, regional or local delivery methods as described herein to form a drug combination in situ.

Infiltration of Drug Combinations or Individual Components Thereof into the Tissue Surrounding a Device or Implant

Alternatively, the tissue cavity or surgical pocket into which a device or implant is placed can be treated with a fibrosis-inhibiting drug combination (or individual component(s) thereof) or a composition that comprises a fibrosis-inhibiting drug combination (or individual component(s) thereof) prior to, during, or after the procedure. This can be accomplished in several ways including: (a) topical application of the drug combination (or individual component(s) thereof) into the anatomical space or surface where the device will be placed (particularly useful for this embodiment is the use of polymeric carriers which release the drug combination (or individual component(s) thereof) over a period ranging from several hours to several weeks). Compositions that can be used for this application include, e.g., fluids, microspheres, pastes, gels, microparticulates, sprays, aerosols, solid implants and other formulations which release one or more components of the drug combination into the region where the device or implant will be implanted; (b) microparticulate forms of the drug combination (or individual component(s) thereof) are also useful for directed delivery into the implantation site; (c) sprayable collagen-containing formulations such as COSTASIS and crosslinked derivatized poly(ethylene glycol)-collagen compositions (described, e.g., in U.S. Pat. Nos. 5,874,500 and 5,565,519 and referred to herein as “CT3” (both from Angiotech Pharmaceuticals, Inc., Canada), loaded with a drug combination (or individual component(s) thereof, applied to the implantation site (or the implant/device surface)); (d) sprayable PEG-containing formulations such as COSEAL or ADHIBIT (Angiotech Pharmaceuticals, Inc.), SPRAYGEL or DURASEAL (both from Confluent Surgical, Inc., Boston, Mass.), loaded with a drug combination or individual component(s) thereof, applied to the implantation site (or the implant/device surface); (e) fibrin-containing formulations such as FLOSEAL or TISSEEL (both from Baxter Healthcare Corporation, Fremont, Calif.) loaded with a drug combination or individual component(s) thereof, applied to the implantation site (or the implant/device surface); (f) hyaluronic acid-containing formulations such as RESTYLANE or PERLANE (both from Q-Med AB, Sweden), HYLAFORM (Inamed Corporation (Santa Barbara, Calif.)), SYNVISC (Biomatrix, Inc., Ridgefield, N.J.), SEPRAFILM or SEPRACOAT (both from Genzyme Corporation, Cambridge, Mass.) loaded with a drug combination or individual component(s) thereof applied to the implantation site (or the implant/device surface); (g) polymeric gels for surgical implantation such as REPEL (Life Medical Sciences, Inc., Princeton, N.J.) or FLOGEL (Baxter Healthcare Corporation) loaded with an anti-scarring drug combination or individual component(s) thereof applied to the implantation site (or the implant/device surface); (h) orthopedic “cements” used to hold prostheses and tissues in place with an anti-scarring drug combination or individual component(s) thereof applied to the implantation site (or the implant/device surface); (i) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND II (Veterinary Products Laboratories, Phoenix, Ariz.), VETBOND (3M Company, St. Paul, Minn.), HISTOACRYL BLUE (Davis & Geck, St. Louis, Mo.) and ORABASE SMOOTHE-N-SEAL Liquid Protectant (Colgate-Palmolive Company, New York, N.Y.) loaded with a drug combination or individual component(s) thereof, applied to the implantation site (or the implant/device surface); and/or (j) protein-based sealants or adhesives such as BIOGLUE (Cryolife, Inc.) and TISSUEBOND (TissueMed, Ltd.) loaded with a drug combination or individual component(s) thereof, applied to the implantation site (or the implant/device surface).

A preferred polymeric matrix which can be used to help prevent the formation of fibrous tissue, either alone or in combination with a fibrosis inhibiting drug combination or individual component(s) thereof, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a drug combination or individual component(s) thereof or a stand-alone composition to help prevent the formation of fibrous tissue.

In certain embodiments, individual components of drug combinations are combined together before being used to locally infiltrate into a tissue. In certain other embodiments, individual components of drug combinations are used to separately infiltrate a tissue and thus form a drug combination in the tissue.

Additional descriptions of infiltrating tissues around medical devices or implants with the anti-scarring drug combinations (or individual components thereof) of the present invention are provided below in connection of using drug combinations or pharmaceutical compositions of the present invention.

Delivery of Drug Combinations or Individual Components via Medical Devices or Implants

In certain embodiments, the fibrosis-inhibiting drug combinations (or individual components thereof) or compositions comprising fibrosis-inhibiting drug combinations (or individual components thereof) of the present invention may be delivered via medical devices or implants, for example, as a coating or otherwise a component of the devices or implants. The therapeutic agents may, or may not, be released from the devices or implants.

A medical device or implants useful in delivering the therapeutic agents (e.g., fibrosis-inhibiting drug combinations or individual components thereof) may be made by (a) directly affixing to the implant or device a desired therapeutic agent or composition containing the therapeutic agent (e.g., by either spraying or electrospraying the medical implant with a drug and/or carrier (polymeric or non-polymeric)-drug composition to create a film and/or coating on all, or parts of the internal or external surface of the device; by dipping the implant or device into a drug and/or carrier (polymeric or non-polymeric)-drug solution to coat all or parts of the device or implant; or by other covalent or noncovalent attachment of the therapeutic agent to the device or implant surface); (b) by coating the medical device or implant with a substance such as a hydrogel which either contains or which will in turn absorb the desired fibrosis-inhibiting agent or composition; (c) by interweaving a “thread” composed of, or coated with, the fibrosis-inhibiting agent(s) into the medical implant or device (e.g., a polymeric strand composed of materials that inhibit fibrosis or polymers which release a fibrosis-inhibiting agent from the thread); (d) by covering all, or portions of the device or implant with a sleeve, cover, electrospun fabric, or mesh containing a fibrosis-inhibiting agent; (e) constructing all, or parts, of the device or implant itself with the desired agent or composition; (f) otherwise impregnating the device or implant with the desired fibrosis-inhibiting agent or composition; (g) composing all, or parts, of the device or implant from metal alloys that inhibit fibrosis; (h) constructing all, or parts of the device or implant itself from a degradable or non-degradable polymer that releases one or more fibrosis-inhibiting drug combinations (or individual components thereof); (i) utilizing specialized multi-drug releasing medical device systems (for example, U.S. Pat. Nos. 6,527,799; 6,293,967; 6,290,673; 6,241,762, U.S. Application Publication Nos. 2003/0199970A1 and 2003/0167085A1, and PCT Publication WO 03/015664) to deliver fibrosis-inhibiting agents alone or in combination.

In certain embodiments, individual components of drug combinations are combined together before being locally used to coat or otherwise being attached to a medical device. In certain other embodiments, individual components of drug combinations are used to separately coat or otherwise be attached to a medical device to form a drug combination on the device.

Additional descriptions of making or using various medical devices or implants that comprise the therapeutic agents of the present invention are provided below in connection with using the anti-fibrosis drug combinations (or individual components thereof) and pharmaceutical compositions of the present invention.

Delivery of Drug Combinations via Combination of Delivery Methods

As discussed above, in certain embodiments, individual components of drug combinations of the present invention may be separately delivered to a site of need by different methods. For instance, one component may be systemically, regionally, or locally delivered to a tissue while another component may be delivered via infiltrating the tissue. In certain other embodiments, one component may be systemically, regionally, or locally delivered to a tissue, while another component may be delivered via a medical device implanted or to be implanted to the tissue. In certain other embodiments, one component may be delivered via infiltrating the tissue while another component may be delivered via a medical device implanted or to be implanted to the tissue.

In certain related embodiments, the present invention provides a method for implanting a medical device comprising: (a) infiltrating a tissue of a host where the medical device is to be, or has been, implanted with a first compound or a composition comprising a first compound, and (b) implanting the medical device that comprises a second compound or a composition comprising a second compound into the host, wherein the first and second compounds form an anti-scarring drug combination.

Uses of Anti-Scarring Drug Combinations and Compositions

The drug combinations and compositions of the present invention can be used in a variety of different applications. For example, the compositions may be used for (a) preventing tissue adhesions; (b) treating or preventing inflammatory arthritis; (c) prevention of cartilage loss; (d) treating or preventing hypertrophic scars and keloids; (e) treating or preventing vascular disease; (f) combining with medical implants or devices, and (g) infiltrating tissues around medical devices or implants. A more detailed description of several specific applications is given below.

(a) Tissue Adhesions

The present invention provides compositions for use in the prevention of adhesions (e.g., surgical adhesions). The compositions may include anti-fibrosis drug combinations or individual components thereof, which provide pharmacological alteration of cellular and/ or non-cellular processes involved in the development and/or progression of surgical adhesions. Anti-fibrosis drug combinations or individual components thereof are described that can reduce surgical adhesions by inhibiting the formation of fibrous or scar tissue. In another aspect, the present invention provides surgical adhesion barriers that include anti-fibrosis drug combinations or individual components thereof.

Surgical adhesions are abnormal, fibrous bands of scar tissue that can form inside the body as a result of the healing process that follows any open or minimally invasive surgical procedure including abdominal, gynecologic, cardiothoracic, spinal, plastic, vascular, ENT, ophthalmologic, urologic, neuro, or orthopedic surgery. Surgical adhesions are typically connective tissue structures that form between adjacent injured areas within the body. Briefly, localized areas of injury trigger an inflammatory and healing response that culminates in healing and scar tissue formation. If scarring results in the formation of fibrous tissue bands or adherence of adjacent anatomical structures (that should be separate), surgical adhesion formation is said to have occurred. Adhesions can range from flimsy, easily separable structures to dense, tenacious fibrous structures that can only be separated by surgical dissection. While many adhesions are benign, some can cause significant clinical problems and are a leading cause of repeat surgical intervention. Surgery to breakdown adhesions (adhesiolysis) often results in failure and recurrence because the surgical trauma involved in breaking down the adhesion triggers the entire process to repeat itself. Surgical breakdown of adhesions is a significant clinical problem and it is estimated that there were 473,000 adhesiolysis procedures in the US in 2002. According to the Diagnosis-Related Groups (DRGs), the total hospital charges for these procedures is likely to be at least US $10 billion annually.

Since all interventions involve a certain degree of trauma to the operative tissues, virtually any procedure (no matter how well executed) has the potential to result in the formation of clinically significant adhesion formation. Adhesions can be triggered by surgical trauma such as cutting, manipulation, retraction or suturing, as well as from inflammation, infection (e.g., fungal or mycobacterium), bleeding or the presence of a foreign body. Surgical trauma may also result from tissue drying, ischemia, or thermal injury. Due to the diverse etiology of surgical adhesions, the potential for formation exists regardless of whether the surgery is done in a so-called minimally invasive fashion (e.g., catheter-based therapies, laparoscopy) or in a standard open technique involving one or more relatively large incisions. Although a potential complication of any surgical intervention, surgical adhesions are particularly problematic in GI surgery (causing bowel obstruction), gynecological surgery (causing pain and/or infertility), tendon repairs (causing shortening and flexion deformities), joint capsule procedures (causing capsular contractures), and nerve and muscle repair procedures (causing diminished or lost function).

Surgical adhesions may cause various, often serious and unpredictable clinical complications; some of which manifest themselves only years after the original procedure was completed. Complications from surgical adhesions are a major cause of failed surgical therapy and are the leading cause of bowel obstruction and infertility. Other adhesion-related complications include chronic back or pelvic pain, intestinal obstruction, urethral obstruction and voiding dysfunction. Relieving the post-surgical complications caused by adhesions generally requires another surgery. However, the subsequent surgery is further complicated by adhesions formed as a result of the previous surgery. In addition, the second surgery is likely to result in further adhesions and a continuing cycle of additional surgical complications.

The placement of medical devices and implants also increases the risk that surgical adhesions will occur. In addition to the above mechanisms, an implanted device can trigger a “foreign body” response where the immune system recognizes the implant as foreign and triggers an inflammatory reaction that ultimately leads to scar tissue formation. A specific form of foreign body reaction in response to medical device placement is complete enclosure (“walling off”) of the implant in a capsule of scar tissue (encapsulation). Fibrous encapsulation of implanted devices and implants can complicate any procedure, but breast augmentation and reconstruction surgery, joint replacement surgery, hernia repair surgery, artificial vascular graft surgery, stent placement, and neurosurgery are particularly prone to this complication. In each case, the implant becomes encapsulated by a fibrous connective tissue capsule which compromises or impairs the function of the surgical implant (e.g., breast implant, artificial joint, surgical mesh, vascular graft, stent or dural patch).

Adhesions generally begin to form within the first several days after surgery. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation may be inhibited.

Various modes of adhesion prevention have been examined, including (1) prevention of fibrin deposition, (2) reduction of local tissue inflammation and (3) removal of fibrin deposits. Fibrin deposition is prevented through the use of physical barriers that are either mechanical or comprised of viscous solutions. Barriers have the added advantage of physically preventing adjacent tissues from contacting each other and thereby reducing the probability that they will scar together. Although many investigators and commercial products utilize adhesion prevention barriers, a number of technical difficulties exist and significant failure rates have been reported. Inflammation is reduced by the administration of drugs such as corticosteroids and non-steroidal anti-inflammatory drugs. However, the results from the use of these drugs in animal models have not been encouraging due to the extent of the inflammatory response and dose restriction due to systemic side effects. Finally, the removal of fibrin deposits has been investigated using proteolytic and fibrinolytic enzymes. A potential complication to the clinical use of these enzymes is the possibility for post-surgical excessive bleeding (surgical hemostasis is critical for procedural success).

Numerous polymeric compositions for use in the prevention of surgical adhesions (e.g., surgical adhesion barriers) may be used in the practice of the invention in combination with an anti-fibrosis drug combination or individual component(s) thereof. In certain embodiments, polymeric compositions can themselves help prevent the formation of fibrous tissue at a surgical site. In certain embodiments, the polymer composition can form a barrier between the tissue surfaces or organs.

For example, the surgical adhesion barrier may be coated onto tissue surfaces and may be composed of an aqueous solution of a hydrophilic, polymeric material (e.g., polypeptides or polysaccharide) having greater than 50,000 molecular weight and a concentration range of 0.01% to 15% by weight. See e.g., U.S. Pat. No. 6,464,970. The surgical adhesion barrier may be a crosslinkable system with at least three reactive compounds each having a polymeric molecular core with at least one functional group. See e.g., U.S. Pat. No. 6,458,889. The surgical adhesions barrier may be composed of a non-gelling polyoxyalkylene composition with or without a therapeutic agent. See e.g., U.S. Pat. No. 6,436,425. The surgical adhesions barrier may be composed of an anionic polymer having an acid sulfate and sulfur content greater than 5% which acts to inhibit monocyte or macrophage invasion. See e.g., U.S. Pat. No. 6,417,173. The surgical adhesions barrier may be an aqueous composition including a surfactant, pentoxifylline and a polyoxyalkylene polyether. See e.g., U.S. Pat. No. 6,399,624. The surgical adhesions barrier may be composed by crosslinking two synthetic polymers, one having nucleophilic groups and the other having electrophilic groups, such that they form a matrix that may be used to incorporate a biologically active compound. See e.g., U.S. Pat. Nos. 6,323,278; 6,166,130; 6,051,648 and 5,874,500. The surgical adhesion barrier may be composed of hyaluronic acid compositions such as those described in U.S. Pat. Nos. 6,723,709; 6,531,147; and 6,464,970. The surgical adhesions barrier may be a polymeric tissue coating which is formed by applying a polymerization initiator to the tissue and then covering it with a water-soluble macromer that is polymerizable using free radical initiators under the influence of UV light. See e.g., U.S. Pat. Nos. 6,177,095 and 6,083,524. The surgical adhesions barrier may be composed of fluent prepolymeric material that is emitted to the tissue surface and then exposed to activating energy in situ to initiate conversion of the applied material to non-fluent polymeric form. See e.g., U.S. Pat. Nos. 6,004,547 and 5,612,050. The surgical adhesions barrier may be a hydrogel-forming, self-solvating, absorbable polyester copolymers capable of selective, segmental association into compliant hydrogels mass upon contact with an aqueous environment. See e.g., U.S. Pat. No. 5,612,052. The surgical adhesions barrier may be an anionic polymer effective to inhibit cell invasion or fibrosis (e.g., dermatan sulfate, dextran sulfate, pentosan polysulfate, or alginate), and a pharmaceutically effective carrier, in which the carrier may be semi-solid. See e.g., U.S. Pat. Nos. 6,756,362; 6,127,348 and 5,994,325. The surgical adhesions barrier may be an acidified hydrogel comprising a carboxypolysaccharide and a polyether having a pH in the range of about 2.0 to about 6.0. See e.g., U.S. Pat. No. 6,017,301. The surgical adhesions barrier may be composed of dextran sulfate having a molecular weight about 40,000 to 500,000 Daltons which is used to inhibit neurite outgrowth. See e.g., U.S. Pat. No. 5,705,178. The surgical adhesions barrier may be a fragmented biocompatible hydrogel which is at least partially hydrated and is substantially free from an aqueous phase, wherein said hydrogel comprises gelatin and will absorb water when delivered to a moist tissue target site. See e.g., U.S. Pat. No. 6,066,325. The surgical adhesions barrier may be a water-soluble, degradable macromer that is composed of at least two-crosslinkable substituents that may crosslink to other macromers at a localized site when under the influence of a polymerization initiator. See e.g., U.S. Pat. No. 6,465,001. The surgical adhesions barrier may be a biocompatible adhesive composition comprising at least one alkyl ester cyanoacrylate monomer and a polymerization initiator or accelerator. See e.g., U.S. Pat. No. 6,620,846.

In one embodiment, the polymers that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting drug combination (or individual component(s) thereof) may be incorporated directly into either the 4 armed NHS-derivatized polyethylene glycol, the acidic solution or the basic buffer. In another embodiment, the fibrosis-inhibiting drug combination (or individual component(s) thereof) may be incorporated into a secondary carrier that may then be incorporated into the 4 armed NHS-derivatized polyethylene glycol, the acidic solution and/or the basic buffer. Secondary carriers may include microparticles and/or microspheres which are made from degradable polymers. Degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. Secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

A preferred polymeric matrix which can be used to help prevent the formation of fibrous tissue, either alone or in combination with a fibrosis inhibiting drug combination (or individual component(s) thereof)/composition that comprising a fibrosis inhibiting drug combination (or individual component(s) thereof), is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for an anti-scarring drug combination (or individual component(s) thereof) or a stand-alone composition to help prevent the formation of fibrous tissue.

Surgical adhesion barriers, which may be combined with an anti-fibrosis drug combination or individual component(s) thereof according to the present invention, also include commercially available products. Examples of surgical adhesion barrier compositions into which a fibrosis agent can be incorporated include: (a) sprayable collagen-containing formulations such as COSTASIS or CT3 (Angiotech Pharmaceuticals, Inc., Canada); (b) sprayable PEG-containing formulations such as COSEAL or ADHIBIT (Angiotech Pharmaceuticals, Inc.), SPRAYGEL or DURASEAL (both from Confluent Surgical, Inc., Boston, Mass.) or FOCALSEAL (Genzyme Corporation, Cambridge, Mass.); (c) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation), (d) fibrinogen-containing formulations such as FLOSEAL or TISSEAL (both from Baxter Healthcare Corporation, Fremont, Calif.); (e) polymeric gels such as REPEL (Life Medical Sciences, Inc., Princeton, N.J.) or FLOWGEL (Baxter Healthcare Corporation, Deerfield, Ill.), (f) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.); (g) dextran sulfate gels such as the ADCON range of products (available from Wright Medical Technology, Inc. Arlington, Tenn.), (h) lipid based compositions such as ADSURF (Britannia Pharmaceuticals Ltd., United Kingdom) and (j) film compositions such as INTERCEED (Ethicon, Inc., Somerville, N.J.) and HYDROSORB (MacroPore Biosurgery, Inc., San Diego, Calif./Medtronic Sofamor Danek, Memphis, Tenn.).

For greater clarity, several specific applications and treatments will be described in greater detail including:

i) Adhesion Prevention in Spinal and Neurosurgical Procedures

Back pain is the number one cause of healthcare expenditures in the United States and accounts for over $50 billion in costs annually ($100 billion worldwide). Over 12 million people in the U.S. have some form of degenerative disc disease (DDD) and 10% of them (1.2 million) will require surgery to correct their problem.

In healthy individuals, the vertebral column is composed of vertebral bone plates separated by intervertebral discs that form strong joints and absorb spinal compression during movement. The intervertebral disc is comprised of an inner gel-like substance called the nucleus pulposus which is surrounded by a tough fibrocartilagenous capsule called the annulus fibrosis. The nucleus pulposus is composed of a loose framework of collagen fibrils and connective tissue cells (resembling fibroblasts and chondrocytes) embedded in a gelatinous matrix of glycosaminoglycans and water. The annulus fibrosus is composed of numerous concentric rings of fibrocartilage that anchor into the vertebral bodies. The most common cause of DDD occurs when tears in the annulus fibrosis create an area of localized weakness that allow bulging, herniation or sequestration of the nucleus pulposis and annulus fibrosis into the spinal canal and/or spinal foramena. The bulging or herniated disc often compresses nerve tissue such as spinal cord fibers or spinal cord nerve root fibers. Pressure on the spinal cord or nerve roots from the damaged intervertebral disc results in neuronal dysfunction (numbness, weakness, tingling), crippling pain, bowel or bladder disturbances and can frequently cause long-term disability. Although many cases of DDD will spontaneously resolve, a significant number of patients will require surgical intervention in the form of minimally invasive procedures, microdiscectomy, major surgical resection of the disc, spinal fusion (fusion of adjacent vertebral bone plates using various techniques and devices), and/or implantation of an artificial disc. The present invention provides for the application of an anti-adhesion or anti-fibrosis drug combination (or individual component(s) thereof) in the surgical management of DDD.

Spinal disc removal is mandatory and urgent in cauda equine syndrome when there is a significant neurological deficit; particularly bowel or bladder dysfunction. It is also performed electively to relieve pain and eliminate lesser neurological symptoms. The spinal nerve roots exit the spinal canal through bony spinal foramena (a bony opening between the vertebra above and the vertebra below) that is a common site of nerve entrapment. To gain access to the spinal foramen during back surgeries, vertebral bone tissue is often resected; a process known as laminectomy.

In open surgical resection of a ruptured lumbar disc or entrapped spinal nerve root (laminectomy) the patient is placed in a modified kneeling position under general anesthesia. An incision is made in the posterior midline and the tissue is dissected away to expose the appropriate interspace; the ligamentum flavum is dissected and in some cases portions of the bony lamina are removed to allow adequate visualization. The nerve root is carefully retracted away to expose the herniated fragment and the defect in the annulus. Typically, the cavity of the disc is entered from the tear in the annulus and the loose fragments of the nucleus pulposus are removed with pituitary forceps. Any additional fragments of disc sequestered inside or outside of the disc space are also carefully removed and the disc space is forcefully irrigated to remove to remove any residual fragments. If tears are present in the dura, the dura is closed with sutures that are often augmented with fibrin glue. The tissue is then closed with absorbable sutures.

Microlumbar disc excision (microdiscectomy) can be performed as an outpatient procedure and has largely replaced laminectomy as the intervention of choice for herniated discs or root entrapment. A one inch incision is made from the spinous process above the disc affected to the spinous process below. Using an operating microscope, the tissue is dissected down to the ligamentum flavum and bone is removed from the lamina until the nerve root can be clearly identified. The nerve root is carefully retracted and the tears in the annulus are visualized under magnification. Microdisc forceps are used to remove disc fragments through the annular tear and any sequestered disc fragments are also removed. As with laminectomy, the disc space is irrigated to remove any disc fragments, any dural tears are repaired and the tissue is closed with absorbable sutures. It should be noted that anterior (abdominal) approaches can also be used for both open and endoscopic lumbar disc excision. Cervical and thoracic disc excisions are similar to lumbar procedures and can also be performed from a posterior approach (with laminectomy) or as an anterior discectomy with fusion.

Back surgeries, such as laminectomies, discectomies and microdiscectomies, often leave the spinal dura exposed and unprotected. As a result, scar tissue frequently forms between the dura and the surrounding tissue. This scar is formed from the damaged erector spinae muscles that overlay the laminectomy site. The result is adhesion development between the muscle tissue and the fragile dura, thereby, reducing mobility of the spine and the nerve roots that exit from it, leading to pain, persistent. neurological symptoms and slow post-operative recovery. Similarly, adhesions that occur in the epidural and dural tissue cause complications in spinal injury (e.g., compression and crush injuries) cases. In addition, scar and adhesion formation within the dura and around nerve roots has been implicated in rendering subsequent (revision and repeat) spine operations technically more difficult to perform.

To circumvent adhesion development, a scar-reducing barrier that comprises an anti-fibrosis drug combination or individual component(s) thereof may be inserted between the dural sleeve and the paravertebral musculature post-laminectomy. Alternatively (or in addition to this), the adhesion barrier can be coated on (or infiltrated into the tissues around) the spinal nerve as it exits the spinal canal and traverses the space between the bony vertebra (i.e., the laminectomy site). This reduces cellular and vascular invasion into the epidural space from the overlying muscle and exposed cancellous bone and thus, reduces the complications associated with scarring of the canal housing, spinal chord and/or nerve roots. In microdiscectomy procedures it is important that the barrier be deliverable as a spray, gel or fluid material that can be administered via the delivery port of an endoscope. Once again, the adhesion barrier can be sprayed onto the spinal nerve (or infiltrated into the tissues around it) as it exits the spinal canal and traverses the space between the bony vertebra (i.e., the laminectomy site). The present invention discloses barrier compositions that can be delivered during surgical disc resection and microdiscectomy either directly, using specialized delivery catheters, via an endoscope, or through a needle or other applicator. When dural defects are present, the fibrosis-inhibiting drug combination or individual component(s) the barrier compositions will assist in the healing of the dura and prevent complications such as blockage of CSF flow.

In another aspect, adhesion formation may be associated with a neurosurgical (brain) procedure. Neurosurgical procedures are fraught with potentially severe post-operative complications that are often attributed to surgical trauma and unwanted fibrosis or gliosis (gliosis is scar tissue formation in the brain as a result of glial cell activity). Increased intracranial bleeding, infection, cerebrospinal fluid leakage and pain are but some complications resulting from adhesions following neurosurgery. For example, if scar tissue interrupts the normal circulation of cerebrospinal fluid (CSF) following brain or spinal surgery, the fluid can accumulate and exert pressure on surrounding tissues (causing increased intracranial pressure) leading to severe complications (such as uncal herneation, brain damage and/or death). Here the adhesion barrier that comprises an fibrosis-inhibiting drug combination or individual component(s) thereof, can be used to prevent excessive dural scarring and adhesion formation in a variety of neurosurgical procedures.

There are numerous compositions loaded with an anti-scarring drug combination or individual component(s) thereof that may be applied to a spinal or neurosurgical site (or to an implant surface placed in the spine—such as an artificial disc, rods, screws, spinal cages, drug-delivery pumps, neurostimulation devices; or to an implant placed in the brain—such as drains, shunts, drug-delivery pumps, neurostimulation devices) for the prevention of surgical adhesions in neurosurgical procedures. In certain embodiments, certain polymeric compositions can themselves help prevent the formation of fibrous tissue at a spinal or neurosurgical site.

Various polymeric compositions can be infiltrated into the spinal or neurosurgical site (e.g., onto tissue at the surgical site or in the vicinity of the implant-tissue interface) for the prevention of surgical adhesions.

In one embodiment, the polymers that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The anti-fibrosis drug combination or individual component(s) thereof may be incorporated directly into either the 4 armed NHS-derivatized polyethylene glycol, the acidic solution or the basic buffer.

In another embodiment, the fibrosis-inhibiting drug combination or individual component(s) thereof may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

In another embodiment, the tissue reactive polymer may be applied initially, and the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

A preferred polymeric matrix which can be used to help prevent the formation of fibrous tissue that leads to surgical adhesions, either alone or in combination with a fibrosis inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for an anti-scarring drug combination (or individual component(s) thereof) or a stand-alone composition to help prevent the formation of fibrous tissue.

Other examples of polymeric compositions that can be infiltrated into the spinal or neurosurgical site (e.g., onto tissue at the surgical site or in the vicinity of the implant-tissue interface) for the prevention of surgical adhesions, include a variety of commercial products. For example, Confluent Surgical, Inc. makes their DURASEAL which is a synthetic hydrogel designed to augment sutured dura closures following cranial surgical procedures. Products that are being developed by Confluent Surgical, Inc. are described in, for example, U.S. Pat. No. 6,379,373. FzioMed, Inc. (San Luis Obispo, Calif.) makes OXIPLEX/SP Gel which is being sold as an adhesion barrier for spine surgery. OXIPLEX/SP Gel is being used for the reduction of pain and radiculopathy in laminectomy, laminotomy and discectomy surgeries. Products being developed by FzioMed, Inc. are described in, for example, U.S. Pat. Nos. 6,566,345 and 6,017,301. Anika Therapeutics, Inc. (Woburn, Mass.) is developing INCERT-S for the prevention of internal adhesions or scarring following spinal surgery. INCERT-S is part of a potential family of bioabsorbable, chemically modified hyaluronic acid therapies. Products being developed by Anika Therapeutics, Inc. are described in, for example, U.S. Pat. Nos. 6,548,081; 6,537,979; 6,096,727; 6,013,679; 5,502,081 and 5,356,883. Life Medical Sciences, Inc. (Little Silver, N.J.) is developing RELIEVE as a bio-resorbable polymer designed to prevent or reduce the formation of adhesions that can follow spinal surgery. Products being developed by Life Medical Sciences, Inc. are described in, for example, U.S. Pat. Nos. 6,696,499; 6,399,624; 6,211,249; 6,136,333 and 5,711,958. Wright Medical Technology, Inc. is selling the ADCON range of products which are dextran sulfate gels originally developed by Gliatech, Inc. (Beachwood, Ohio.) to inhibit postsurgical peridural fibrosis that occurs in posterior lumbar laminectomy or laminotomy procedures where nerve routes are exposed. ADCON provides a barrier between the spinal cord and nerve roots and the surrounding muscle and bone following lumbar spine surgeries. The ADCON range of products may be described in, for example, U.S. Pat. Nos. 6,417,173; 6,127,348; 6,083,930; 5,994,325 and 5,705,178.

Other commercially available materials that may be loaded with an anti-fibrosis drug combination or individual component(s) thereof and applied to or infiltrated into a spinal or neurosurgical site (or to an implant surface) for the prevention of adhesions include: (a) sprayable collagen-containing formulations such as COSTASIS or CT3; (b) sprayable PEG-containing formulations such as COSEAL, ADHIBIT, FOCALSEAL, or SPRAYGEL; (c) fibrinogen-containing formulations such as FLOSEAL or TISSEAL (both from Baxter Healthcare Corporation, Fremont, Calif.); (d) hyaluronic acid-containing formulations such as RESTYLANE, PERLANE, HYLAFORM, SYNVISC, SEPRAFILM or SEPRACOAT; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (h) lipid based compositions such as ADSURF, and (j) film compositions such as INTERCEED (Ethicon, Inc., Somerville, N.J.) and HYDROSORB (MacroPore Biosurgery, Inc., San Diego, Calif./Medtronic Sofamor Danek, Memphis, Tenn.). It should be obvious to one of skill in the art that commercial compositions not specifically cited above as well as next-generation and/or subsequently-developed commercial products are to be anticipated and are suitable for use under the present invention.

As described above, the compositions for the prevention of surgical adhesions can be applied directly or indirectly to the tissue in a spinal or neurosurgical site.

The compositions can be administered in any manner described herein. Exemplary methods include either direct application at the time of surgery, with endoscopic, ultrasound, CT, MRI, or fluoroscopic guidance, and/or in conjunction with the placement of a device or implant at the surgical site. Representative examples of devices or implants for use in spinal and neurosurgical procedures includes, without limitation, dural patches, spinal prostheses (e.g., artificial discs, injectable filling or bulking agents for discs, spinal grafts, spinal nucleus implants, intervertebral disc spacers), fusion cages, neurostimulation devices, implantable drug-delivery pumps, shunts, drains, electrodes, and bone fixation devices (e.g., anchoring plates and bone screws).

The composition may be applied during open or endoscopic procedures: (a) to the surface of the operative site (e.g., as an injectable, solution, paste, gel, in situ forming gel or mesh) before, during, or after the surgical procedure; (b) to the surface of the tissue surrounding the operative site (e.g., as an injectable, solution, paste, gel, in situ forming gel or mesh) before, during or after the surgical procedure; (c) by topical application of the composition into an anatomical space (such as the subdural space or intrathecally) at the surgical site (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where the device will be inserted); (d) via percutaneous injection into the tissue in and around the operative site as a solution, as an infusate, or as a sustained release preparation; and/or (e) by any combination of the aforementioned methods. Combination therapies (e.g., combinations with antithrombotic, anti-infective, and/or antiplatelet agents) can also be used.

In certain applications involving the placement of a medical device or implant, it may be desirable to apply an anti-fibrosis drug combination (or individual component(s) thereof) or a composition that comprise an anti-fibrosis drug combination (or individual component(s) thereof) at a site that is adjacent to an implant (preferably near the implant-tissue interface). This can be accomplished during open or endoscopic procedures by applying the anti-fibrosis drug combination (or individual component(s) thereof) or the composition that comprise an anti-fibrosis drug combination (or individual component(s) thereof): (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the drug combination (or individual component(s) thereof) or the composition comprising the drug combination (or individual component(s) thereof) into the anatomical space (such as the sudural space or intrathecally) where the implant will be placed (particularly useful for this embodiment is the use of polymeric carriers which release the fibrosis-inhibiting drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where the device will be inserted); (e) via percutaneous injection into the tissue surrounding the implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

In certain embodiments, the anti-fibrosis drug combination (or individual component(s) thereof) or the composition that comprising the anti-fibrosis drug combination (or individual component(s) thereof) may be delivered to the tissue (or device/tissue interface) in the form of a spray or gel during open, endoscopic or catheter-based procedures. The fibrosis-inhibiting drug combination or individual component(s) thereof can be incorporated directly into the surgical adhesion barrier or it can be incorporated into a secondary carrier (polymeric or non-polymeric), as described above, that is then incorporated into the adhesion barrier. Examples of the compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The antifibrosisfibrosis-inhibiting agent(s) may be incorporated directly into either the 4 armed NHS-derivatized polyethylene glycol, the acidic solution or the basic buffer. In another embodiment, the fibrosis-inhibiting drug combination (or individual component(s) thereof) may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination (or individual component(s) thereof) may then be applied to the coated tissue. The fibrosis-inhibiting drug combination (or individual component(s) thereof) may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The fibrosis-inhibiting drug combination (or individual component(s) thereof) may be incorporated directly into either the 4 armed NHS-derivatized polyethylene glycol, or the basic buffer. In another embodiment, the fibrosis-inhibiting drug combination or individual component(s) may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof

ii) Adhesion Prevention in Gynecological Procedures

In certain embodiments, adhesion formation may be associated with a gynecological surgical procedure. The post-operative adhesions occur in 60 to 90% of patients undergoing major gynecologic surgery and represent one of the most common causes of infertility in the industrialized world. Adhesions can form between the ovaries, the fallopian tubes, the bowel or the walls of the pelvis. Fibrous bands can connect to the normally mobile adnexal structures (ovaries and fallopian tubes) to other tissues, causing them to lose mobility, kink or twist. If the adhesions tighten around, constrict or twist the fallopian tubes themselves, they can block the passage of an ovum from the ovaries into and through the fallopian tube leading to infertility. Adhesions around the fallopian tubes can also interfere with sperm transport to the ovum and also cause infertility. Other adhesion-related complications include chronic pelvic pain, dysparunia, urethral obstruction and voiding dysfunction.

Several products are available commercially or under development for the management of gynecological adhesions. Life Medical Sciences, Inc. is producing the products, REPEL, REPEL-CV, RESOLVE and RELIEVE that are in various stages of development and may be used to prevent surgical adhesions in gynecological and other surgeries. Products being developed by Life Medical Sciences, Inc. are described in, for example, U.S. Pat. Nos. 6,696,499; 6,399,624; 6,211,249; 6,136,333 and 5,711,958. Confluent Surgical, Inc. makes their SPRAYGEL which is a unique sprayable adhesion barrier that is being developed for use in pelvic and intrauterine surgical procedures. Products that are being developed by Confluent Surgical, Inc. are described in, for example, U.S. Pat. No. 6,379,373. Closure Medical Corp. (Raleigh, N.C.) is developing a cyanoacrylate-based internal adhesives that may be used to seal internal surgical incisions or grafts which may be compatible in gynecology and general surgical specialties. Products that are being developed by Closure Medical, Corp. are described in, for example, U.S. Pat. Nos. 6,620,846; 6,579,469; 6,565,840; 6,547,467 and 5,981,621.

Other commercially available materials that may be loaded with an anti-fibrosis composition or individual component(s) thereof and applied to or infiltrated into a gynecological surgical site (or to the surface of a device or implant) for the prevention of adhesions in open or endoscopic gynecologic surgery include: (a) sprayable collagen-containing formulations such as COSTASIS or CT3; (b) sprayable PEG-containing formulations such as COSEAL, ADHIBIT, FOCALSEAL or DURASEAL; (c) fibrinogen-containing formulations such as FLOSEAL or TISSEAL; (d) hyaluronic acid-containing formulations such as RESTYLANE or PERLANE, HYLAFORM, SYNVISC, SEPRAFILM or SEPRACOAT; (e) polymeric gels for surgical implantation such as FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. It should be obvious to one of skill in the art that commercial compositions not specifically cited above as well as next-generation and/or subsequently-developed commercial products are to be anticipated and are suitable for use under the present invention.

Gynecological procedures are performed for a variety of medical conditions including hysterectomy (removal of the uterus), myomectomy (removal of uterine fibroids), endometriosis (ablation procedures), infertility (in vitro fertilization, adhesiolysis), birth control (tubal ligation), reversal of sterilization, pain, dysmennorrhea, dysfunctional uterine bleeding, ectopic pregnancy, ovarian cysts, gynecologic malignancies and numerous other conditions. Although many procedures are still performed through open surgical techniques, increasingly, gynecologic surgery is performed via an endoscope inserted through the umbilicus (belly button). Virtually any manipulation of the pelvic organs or pelvic sidewall can trigger a cascade that ultimately results in the formation of pelvic adhesions. In many instances, the adhesions must be broken down during a repeat surgical intervention for the treatment of pain or infertility. An adhesion barrieris best applied directly to the affected areas (as a solid, a film, a paste, a gel, a liquid or another such formulation) during the open or endoscopic procedure. In a preferred embodiment, the barrier is sprayed under direct endoscopic vision during the procedure onto the pelvic organs (and bowel, pelvic and abdominal sidewall) that are operated on, or manipulated, during the intervention. Since adhesions often occur in areas at a distance from the tissues actually instrumented during a surgical intervention, it is recommended that the barrier be applied to a wide area in the pelvis (potentially even the entire adnexa, pelvic sidewall and pelvic surface of the uterus). Preferred barriers include liquids, gels, pastes, sprays or other formulations that can be delivered through an endoscope, adhere to the tissues treated, and remain in place long enough to deliver the anti-fibrosis drug combination or individual component(s) thereof and/or prevent adhesion formation. As an alternative, the anti-fibrosis drug combination or individual component(s) thereof can be delivered directly into the peritoneal cavity as an injectable (either before, during or after the procedure) such that the drug is delivered in doses high enough and long enough (multiple dosing and/or sustained release preparations are preferred) to prevent adhesions and the complications arising from them. An ideal adhesion therapy will reduce the incidence, number and tenacity of adhesions and improve patient outcome by reducing pain, improving fertility and limiting the need for repeat interventions.

As described above, the compositions for the prevention of surgical adhesions can be applied directly or indirectly to the tissue in a gynecological site. The anti-fibrosis compositions can be administered in any manner described herein. Exemplary methods include either direct application at the time of surgery or with endoscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in gynecological procedures includes, without limitation, genital-urinary stents, bulking agents, sterilization devices (e.g., valves, clips and clamps), and tubal occlusion implants and plugs.

The anti-fibrosis drug combination (or individual component(s) thereof) or the composition comprising the anti-fibrosis drug combination (or individual component(s) thereof) may be applied during open or endoscopic gynecological surgery: (a) to the tissue surface of the pelvic side wall, adnexa, uterus and any adjacent affected tissues (e.g., as an injectable, solution, paste, gel, in situ forming gel or mesh) during the surgical procedure; (b) to the surface of an implanted device or implant and/or the tissue surrounding the implant (e.g., as an injectable, solution, paste, gel, in situ forming gel or mesh) before, during, or after the surgical procedure; (c) by intraperitoneal or endoscopic injection of the composition into the anatomical space (i. e., the peritoneal or pelvic cavity) at the surgical site (particularly useful for this embodiment is the use of injectable compositions containing polymeric carriers which release the fibrosis-inhibiting drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where there is a risk of adhesion formation); (d) via percutaneous injection into the tissue as a solution as an infusate or as a sustained release preparation; (e) by guided catheter or hysteroscopic injection of the composition into the lumen of the fallopian tubes (i.e., inserting a catheter or an endoscope via the vagina, cervix and uterus until it can be advanced into the lumen of the fallopian tube) at the desired tubal location (particularly useful for this embodiment is the use of injectable compositions containing polymeric carriers which release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) can be delivered into the areas of the fallopian tube where there is a risk of adhesion formation); and/or (f) by any combination of the aforementioned methods. Combination therapies (e.g., combinations with antithrombotic, anti-infective, and/or antiplatelet agents) can also be used in the manner described above.

In certain applications involving the placement of a gynecological medical device or implant, it may be desirable to apply the anti-fibrosis drug combination (or individual component(s) thereof) or the composition comprising the anti-fibrosis drug combination (or individual component(s) thereof) at a site that is adjacent to an implant (preferably near the implant-tissue interface). This can be accomplished during open or endoscopic procedures by applying the polymeric composition, with or without a fibrosis-inhibiting drug combination (or individual component(s) thereof): (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (such as the lumen of the fallopian tube, the uterine cavity, the peritoneal cavity, or the pelvic cavity) where the implant will be placed; (e) via percutaneous injection into the tissue surrounding the implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

In certain embodiments, the anti-fibrosis drug combination (or individual component(s) thereof) or the composition comprising the anti-fibrosis drug combination (or individual component(s) thereof) may be delivered to the female pelvic tissue (or device/tissue interface) in the form of a spray or gel during open, endoscopic or catheter-based procedures. The fibrosis-inhibiting drug combination or individual components thereof can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting drug combination or individual component(s) thereof may be incorporated directly into either the 4 armed NHS-derivatized polyethylene glycol, the acidic solution or the basic buffer. In another embodiment, the fibrosis-inhibiting drug combination or individual component(s) thereof may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting drug combination or individual component(s) thereof may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

iii) Adhesion Prevention in Abdominal Procedures

In certain embodiments, adhesions may be associated with an abdominal surgical procedure. Following abdominal surgery, the formation of adhesions may cause loops of intestines become entangled or twisted about fibrous bands of tissue that impair the normal fluid movement of the bowel. The entanglements can cause partial or total flow obstruction through the bowel, scar can constrict around the bowel, volvulus (twisting) can occur, or blood flow to and from the bowel can be compromised. With entanglement, volvulus or fibrous banding the result is typically partial or complete bowel obstruction; a condition that requires immediate decompression, may require surgery and can cause death. Infarction (interruption of blood flow to the bowel) from adhesions or volvulus is a medical emergency that usually requires surgical removal of the affected bowel and can also lead to death if not treated aggressively. Peritoneal adhesions (adhesions between the abdominal wall and the underlying organs) represent another major health care problem causing pain, bowel obstruction and other potentially serious post-operative complications and they are associated with all types of abdominal surgery (incidence of 50-90% for laparotomies).

As described previously, adhesion barriers are frequently used in the management of abdominal adhesions following open or endoscopic procedures. A variety of commercially available adhesion barriers are suitable for combining with a fibrosis-inhibiting drug combination or individual component(s) thereof in the management of abdominal adhesions. Confluent Surgical, Inc. makes their SPRAYGEL which is a unique sprayable adhesion barrier that is being developed for use in abdominal and pelvic surgical procedures. Products that are being developed by Confluent Surgical, Inc. are described in, for example, U.S. Pat. No. 6,379,373. Closure Medical Corp. (Raleigh, N.C.) is developing a cyanoacrylate-based internal adhesives that may be used to seal internal surgical incisions or grafts which may be compatible in gastrointestinal, oncology and general surgical specialties. Products that are being developed by Closure Medical, Corp. are described in, for example, U.S. Pat. Nos. 6,620,846; 6,579,469; 6,565,840; 6,547,467 and 5,981,621. Genzyme Corporation has developed hyaluronic acid-containing biomaterials, such as SEPRAFILM and SEPRACOAT, to reduce the incidence of adhesions following abdominal and pelvic surgeries (see, e.g., U.S. Pat. Nos. 6,780,427; 6,531,147; 6,521,223 and 6,010,692.

Other commercially available materials that may be loaded with an anti-fibrosis composition or individual component(s) thereof and applied to or infiltrated into an abdominal site (or to the surface of an implanted device or implant) for the prevention of adhesions during open or endoscopic abdominal procedures include: (a) sprayable collagen-containing formulations such as COSTASIS or CT3; (b) sprayable PEG-containing formulations such as COSEAL, ADHIBIT, FOCALSEAL or DURASEAL; (c) fibrinogen-containing formulations such as FLOSEAL or TISSEAL; (d) hyaluronic acid-containing formulations such as RESTYLANE or PERLANE, HYLAFORM, or SYNVISC; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. It should be obvious to one of skill in the art that commercial compositions not specifically cited above as well as next-generation and/or subsequently-developed commercial products are to be anticipated and are suitable for use under the present invention.

Abdominal surgical procedures are performed for a variety of medical conditions including hernia repair (abdominal, ventral, inguinal, incisional), bowel obstruction, inflammatory bowel disease (ulcerative colitis, Crohn's disease), appendectomy, trauma (penetrating wounds, blunt tauma), tumor resection, infections (abscesses, peritonitis), cholecystectomy, gastroplasty (bariatric surgery), esophageal and pyloric strictures, colostomy, diversion iliostomy, anal-rectal fistulas, hemorrhoidectomies, splenectomy, hepatic tumor resection, pancreatitis, bowel perforation, upper and lower GI bleeding, and ischemic bowel. Although many procedures are still performed through open surgical techniques, increasingly, abdominal surgery is performed via an endoscope inserted through the umbilicus (belly button). Virtually any manipulation of the abdominal viscera or peritoneum can trigger a cascade that ultimately results in the formation of abdominal adhesions. In many instances, the adhesions must be broken down during a repeat surgical intervention for the treatment of pain or bowel obstruction. An adhesion barrier is best applied directly to the affected areas (as a solid, a film, a paste, a gel, a liquid or another such formulation) during the open or endoscopic procedure. In a preferred embodiment, the barrier is sprayed under direct or endoscopic vision during the procedure onto the abdominal organs (such as the large and small bowel, stomach, liver, spleen, gall bladder etc.), visceral peritoneum and abdominal (wall) peritoneum that are operated on, or manipulated, during the intervention. Since adhesions often occur in areas at a distance from the tissues actually instrumented during a surgical intervention, it is recommended that the barrier be applied to a wide area in the abdomen (potentially even the entire viscera and abdominal wall). Preferred barriers include films, liquids, gels, pastes, sprays or other formulations that can be delivered during open procedures or through an endoscope, adhere to the tissues treated, and remain in place long enough to deliver the therapeutic agent and/or prevent adhesion formation. As an alternative, the anti-fibrosis drug combination or individual component(s) thereof can be delivered directly into the peritoneal cavity as an injectable (either before, during or after the procedure) such that the drug is delivered in doses high enough and long enough (multiple dosing and/or sustained release preparations are preferred) to prevent adhesions and the complications arising from them. An ideal adhesion therapy will reduce the incidence, number and tenacity of adhesions and improve patient outcome by reducing pain, preventing bowel obstruction and limiting the need for repeat interventions.

As described above, the compositions for the prevention of surgical adhesions can be applied directly or indirectly to the tissue in an abdominal procedure. The polymeric compositions can be administered in any manner described herein. Exemplary methods include either direct application at the time of surgery or with endoscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in abdominal procedures includes, without limitation, hernia meshes, restriction devices for obesity, peritoneal dialysis catheters, peritoneal drug-delivery catheters, GI tubes for drainage or feeding, portosystemic shunts, shunts for ascites, gastrostomy or percutaneous feeding tubes, jejunostomy endoscopic tubes, colostomy devices, drainage tubes, biliary T-tubes, hemostatic implants, enteral feeding devices, colonic and biliary stents, low profile devices, gastric banding implants, capsule endoscopes, anti-reflux devices, and esophageal stents.

The anti-fibrosis drug combination (or individual component(s) thereof) or the composition that comprises the anti-fibrosis drug combination (or individual component(s) thereof) may be applied during open or endoscopic abdominal surgery: (a) to the tissue surface of the peritoneal cavity, visceral peritneum, abdominal organs, abdominal wall and any adjacent affected tissues (e.g., as an injectable, solution, paste, gel, in situ forming gel or mesh) during the surgical procedure; (b) to the surface of an implanted device or implant and/or the tissue surrounding the implant (e.g., as an injectable, solution, paste, gel, in situ forming gel or mesh) before, during, or after the surgical procedure; (c) by intraperitoneal or endoscopic injection of the composition into the anatomical space (i.e., the peritoneal cavity) at the surgical site (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where there is a risk of adhesion formation); (d) via percutaneous injection into the tissue as a solution as an infusate or as a sustained release preparation; (e) by guided catheter or endoscopic (gastroscope, ERCP, colonoscope) injection of the composition into the lumen of the GI tract at the desired location (particularly useful for this embodiment is the use of injectable compositions containing polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) can be delivered into the areas of the GI tract where there is a risk of adhesion formation); and/or (f) by any combination of the aforementioned methods. Combination therapies (e.g., combinations with antithrombotic, anti-infective, and/or antiplatelet agents) can also be used in the manner described above.

In certain applications involving the placement of an abdominal or gastrointestinal medical device or implant, it may be desirable to apply the anti-fibrosis drug combination or individual component(s) thereof at a site that is adjacent to an implant (preferably near the implant-tissue interface). This can be accomplished during open or endoscopic procedures by applying the polymeric composition, with or without a fibrosis-inhibiting drug combination (or individual component(s) thereof): (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (such as the lumen of the GI tract or the peritoneal cavity) where the implant will be placed (particularly useful for this embodiment is the use of polymeric carriers that release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations that release the drug combination or individual component(s) thereof and can be delivered into the region where the device will be inserted); (e) via percutaneous injection into the tissue surrounding the implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

In certain embodiments, the anti-fibrosis drug combination (or individual component(s) thereof) or the composition comprising the anti-fibrosis drug combination (or individual component(s) thereof) may be delivered to the abdomen (or device/tissue interface) in the form of a spray or gel during open, endoscopic or catheter-based procedures. The fibrosis-inhibiting drug combination or individual component(s) thereof can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting drug combination (or individual component(s) thereof) may be incorporated directly into either the 4 armed NHS-derivatized polyethylene glycol, the acidic solution or the basic buffer. In another embodiment, the fibrosis-inhibiting drug combination or individual component(s) thereof may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially, and the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g. pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting drug combination or individual component(s) thereof may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially, and the fibrosis-inhibiting drug combination or individual components thereof may then be applied to the coated tissue. The fibrosis-inhibiting drug combination or individual components thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

iv) Adhesion Prevention in Cardiac Procedures

In certain embodiments, adhesions may be associated with a cardiac surgical procedure. In the case of cardiac surgery involving transplants, vascular repair, coronary artery bypass grafting (CABG), congenital heart defects, and valve replacements, staged procedures and reoperations (particularly repeat CABG surgery) are very common. As such, cardiac surgeons frequently must operate on tissues that have been surgically traumatized previously and have thick fibrous adhesions present which make dissection difficult. Post-operative pericardial adhesions (adhesions between the two surfaces of the pericardial sac) from initial surgery are common. Pericardial adhesions can cause symptoms by restricting the normal movement and filling of the heart during the cardiac cycle and can subject patients undergoing repeat cardiac surgery to elevated procedural risks. Resternotomy (re-opening the chest wall incision and surgical exposure of the heart) and dissection of the adhesions that accompany it, increases the risk of potential injury to the heart, great vessels and extracardiac grafts, increases operative time (including increasing the time the patient is on heart-lung bypass), and can increase procedural morbidity and mortality. Resternotomy is associated with as much as a 6% incidence of major vascular injury and a greater than 35% mortality has been reported for patients experiencing major hemorrhage during resternotomy. A 50% mortality has been reported for associated injuries to aortocoronary grafts. Staged pediatric open-heart surgery (repeat procedures required as the heart grows) is also associated with a very high incidence of complications due to reoperations.

As described previously, adhesion barriers are frequently used in the management of adhesions following open-heart procedures. A variety of commercially available adhesion barriers are suitable for combining with a fibrosis-inhibitor (and/or an anti-infective agent) in the management of cardiac surgery adhesions. Life Medical Sciences, Inc. is developing the products, REPEL, REPEL-CV, RESOLVE and RELIEVE that are in various stages of development and may be used to prevent surgical adhesions of open heart and other surgeries. Products being developed by Life Medical Sciences, Inc. are described in, for example, U.S. Pat. Nos. 6,696,499; 6,399,624; 6,211,249; 6,136,333 and 5,711,958. Closure Medical Corp. (Raleigh, N.C.) is developing a cyanoacrylate-based internal adhesives that may be used to seal internal surgical incisions or grafts which may be compatible in pulmonary and general surgical specialties. Products that are being developed by Closure Medical, Corp. are described in, for example, U.S. Pat. Nos. 6,620,846; 6,579,469; 6,565,840; 6,547,467 and 5,981,621. Genzyme Corporation has developed hyaluronic acid-containing biomaterials, such as SEPRAFILM and SEPRACOAT, to reduce the incidence of adhesions following cardiothoracic surgeries (see, e.g., U.S. Pat. Nos. 6,780,427; 6,531,147; 6,521,223 and 6,010,692.

Other commercially available materials that may be loaded with an anti-fibrosis composition or individual component(s) thereof and applied to or infiltrated into cardiac surgery site (or to the surface of an implanted device or implant) for the prevention of adhesions during open or endoscopic heart surgery include: (a) sprayable collagen-containing formulations such as COSTASIS or CT3; (b) sprayable PEG-containing formulations such as COSEAL, ADHIBIT, FOCALSEAL or DURASEAL; (c) fibrinogen-containing formulations such as FLOSEAL or TISSEAL; (d) hyaluronic acid-containing formulations such as RESTYLANE or PERLANE, HYLAFORM, or SYNVISC; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. It should be obvious to one of skill in the art that commercial compositions not specifically cited above as well as next-generation and/or subsequently-developed commercial products are to be anticipated and are suitable for use under the present invention.

Virtually any manipulation of the chest wall, pericardium and heart can trigger a cascade that ultimately results in the formation of adhesions. In many instances, the adhesions must be broken down during repeat open-heart interventions. An adhesion barrier is best applied directly to the affected areas (as a solid, a film, a paste, a gel, a liquid or another such formulation) during open or endoscopic cardiac procedures. In a preferred embodiment, the barrier is sprayed under direct or endoscopic vision during the procedure onto the heart, pericardium, pleura and chest wall that are operated on, or manipulated, during the intervention. Since adhesions often occur in areas at a distance from the tissues actually instrumented during a surgical intervention, it is recommended that the barrier be applied to a wide area in the chest (potentially even the entire cardiopulmonary viscera and infiltrated throughout the pericardial sac). Preferred barriers include films, liquids, gels, pastes, sprays or other formulations that can be delivered during open procedures or through an endoscope, adhere to the tissues treated, and remain in place long enough to deliver the anti-fibrosis drug combination or individual component(s) thereof and/or prevent adhesion formation. As an alternative, the anti-fibrosis drug composition or individual component(s) thereof can be delivered directly into the pericardial sac as an injectable (either before, during or after the procedure) such that the drug is delivered in doses high enough and long enough (multiple dosing and/or sustained release preparations are preferred) to prevent adhesions and the complications arising from them. An ideal adhesion therapy will reduce the incidence, number and tenacity of adhesions and improve patient outcome by reducing the complications of repeat interventions.

As described above, the compositions for the prevention of surgical adhesions can be applied directly or indirectly to the tissue in a cardiac surgery procedure. The anti-fibrosis drug combination (or individual component(s) thereof) or the composition that comprises the anti-fibrosis drug combination (or individual component(s) thereof) can be administered in any manner described herein. Exemplary methods include either direct application at the time of surgery or with endoscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in cardiac procedures includes, without limitation, heart valves (porcine, artificial), ventricular assist devices, cardiac pumps, artificial hearts, stents, bypass grafts (artificial and endogenous), patches, cardiac electrical leads, defibrillators and pacemakers.

The anti-fibrosis drug combination (or individual component(s) thereof) or the composition comprising the anti-fibrosis drug combination (or individual component(s) thereof) may be applied during open or endoscopic heart surgery: (a) to the tissue surface of the pericardium (or infiltrated into the pericardial sac), heart, great vessels, pleura, lungs, chest wall and any adjacent affected tissues (e.g., as an injectable, solution, paste, gel, in situ forming gel or mesh) during the surgical procedure; (b) to the surface of an implanted device or implant and/or the tissue surrounding the implant (e.g., as an injectable, solution, paste, gel, in situ forming gel or mesh) before, during, or after the surgical procedure; (c) by intraperitoneal or endoscopic injection of the composition into the anatomical space (i.e., the pericardial sac) at the surgical site (particularly useful for this embodiment is the use of injectable compositions containing polymeric carriers which release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where there is a risk of adhesion formation); (d) via percutaneous injection into the tissue as a solution as an infusate or as a sustained release preparation (intrapericardial injection); (e) by guided catheter or endoscopic injection of the composition into the lumen or the walls of the atria, ventricles, great vessels, coronary arteries or the pericardial sac (particularly useful for this embodiment is the use of injectable compositions containing polymeric carriers which release the fibrosis-inhibiting drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) can be delivered into the areas of the heart where there is a risk of adhesion formation); and/or (f) by any combination of the aforementioned methods. Combination therapies (e.g., combinations with antithrombotic, anti-infective, and/or antiplatelet agents) can also be used in the manner described above.

In certain applications involving the placement of a cardiac medical device or implant, it may be desirable to apply the anti-fibrosis drug combination or individual component(s) thereof at a site that is adjacent to an implant (preferably near the implant-tissue interface). This can be accomplished during open, endoscopic or catheter-based procedures by applying the anti-fibrosis drug combination (or individual component(s) thereof) or the composition that comprises the anti-fibrosis drug combination (or individual component(s) thereof): (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the implant; (c) to the surface of the implant and the tissue surrounding the implant (e.g., as an injectable, solution, paste, gel, in situ forming gel or mesh) before, during, or after implantation of the implant; (d) by topical application of the drug combination (or individual component(s) thereof) or the composition into the anatomical space (pericardial sac, intracardiac, intra-arterial) where the implant will be placed (particularly useful for this embodiment is the use of polymeric carriers which release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) can be delivered into the region where the device will be inserted); (e) via percutaneous injection into the tissue surrounding the implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

In certain embodiments, the polymeric composition may be delivered to the heart (or device/tissue interface) in the form of a spray or gel during open, endoscopic or catheter-based procedures. The fibrosis-inhibiting drug combination or individual component(s) thereof can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting drug combination or individual component(s) thereof may be incorporated directly into either the 4 armed NHS-derivatized polyethylene glycol, the acidic solution or the basic buffer. In another embodiment, the fibrosis-inhibiting drug combination or individual component(s) thereof may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting drug combination or individual component(s) thereof may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially, and the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

v) Adhesion Prevention in Orthopedic Procedures

In certain embodiments, adhesions may be associated with an orthopedic surgical procedure. Many orthopedic surgical interventions are performed as a result of injury or trauma (fractures; torn ligaments, cartilage, tendons or muscles) that cause significant tissue damage that can lead to excessive scarring and adhesion formation. As a result, orthopedic procedures often result in potentially severe post-operative complications which may be attributed to the trauma which caused the injury or to the trauma from the surgery itself. In general, excessive scarring and adhesion formation in orthopedic conditions follows certain patterns: (a) in joint injuries, it can result in a deformity such that the joint cannot fully extend, flex, or rotate (contractures); (b) in tendon injuries, it can prevent normal movement and lead to shortening; (c) in cartilage injuries, it can lead to the conversion of hyaline cartilage to fibrocartilage with a resultant loss of function and joint instability; (d) in muscle injuries, it can cause adhesion to adjacent tissues, loss of strength and loss of function; (e) in nerve injuries, it can result in loss of conduction and function; if the nerve becomes entrapped (encircled and constricted) by scar, it can cause pain, sensory impairment and loss of motor function; and (f) in tendons and ligaments, it can cause shortening, loss of range of motion and impaired function. The complications of adhesions can be wide spread; for example, adhesions formed after spinal surgery may produce low back pain, leg pain and sphincter disturbance (bladder and bowel). For this reason strategies designed to reduce adhesion formation in musculoskeletal surgery is a significant clinical problem. The local administration of anti-adhesive compositions loaded with a fibrosis-inhibiting drug combination or individual component(s) thereof, can be utilized in a wide array of clinical situations and conditions to improve patient outcomes following emergency or elective orthopedic interventions.

As described previously, adhesion barriers are frequently used in the management of adhesions following orthopedic procedures. A variety of commercially available adhesion barriers are suitable for combining with a fibrosis-inhibiting drug combination or individual component(s) thereof in the management of orthopedic surgery adhesions. Closure Medical Corp. (Raleigh, N.C.) is developing a cyanoacrylate-based internal adhesives that may be used to seal internal surgical incisions or grafts which may be compatible in orthopedic and general surgical specialties. Products that are being developed by Closure Medical, Corp. are described in, for example, U.S. Pat. Nos. 6,620,846; 6,579,469; 6,565,840; 6,547,467 and 5,981,621. Life Medical Sciences, Inc. is developing the products, REPEL, REPEL-CV, RESOLVE and RELIEVE that are in various stages of development and may be used to prevent surgical adhesions in orthopedic and spinal surgeries. Products being developed by Life Medical Sciences, Inc. are described in, for example, U.S. Pat. Nos. 6,696,499; 6,399,624; 6,211,249; 6,136,333 and 5,711,958.

Other commercially available materials that may be used alone, or loaded with a therapeutic agent (e.g., a fibrosis-inhibiting agent or an anti-infective agent), applied to or infiltrated into an orthopedic site (or to the surface of an implanted device or implant) for the prevention of adhesions in open or endoscopic orthopedic surgery include: (a) sprayable collagen-containing formulations such as COSTASIS or CT3; (b) sprayable PEG-containing formulations such as COSEAL, ADHIBIT, FOCALSEAL, SPRAYGEL or DURASEAL; (c) fibrinogen-containing formulations such as FLOSEAL or TISSEAL; (d) hyaluronic acid-containing formulations such as RESTYLANE, HYLAFORM, PERLANE, SYNVISC, SEPRAFILM, SEPRACOAT, INTERGEL, or LUBRICOAT; (e) polymeric gels for surgical implantation such as REPEL or FLOWGEL; (f) orthopedic “cements” used to hold prostheses and tissues in place, such as OSTEOBOND (Zimmer), LVC (Wright Medical Technology), SIMPLEX P (Stryker), PALACOS (Smith & Nephew), and ENDURANCE (Johnson & Johnson, Inc.); (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) implants containing hydroxyapatite (or synthetic bone material such as calcium sulfate, VITOSS (Orthovita) and CORTOSS (Orthovita)); (h) other biocompatible tissue fillers, such as those made by BioCure, 3M Company and Neomend; (i) polysacharride gels such as the ADCON series of gels; (j) films, sponges or meshes such as INTERCEED, VICRYL mesh, and GELFOAM; (o) lipid based compositions such as ADSURF; and (p) OSSIGEL, a viscous formulation of hyaluronic acid (HA) and basic fibroblast growth factor (bFGF) designed to accelerate bone fracture healing (Orquest, Inc.). It should be obvious to one of skill in the art that commercial compositions not specifically cited above as well as next-generation and/or subsequently-developed commercial products are to be anticipated and are suitable for use under the present invention.

Orthopedic surgical procedures are performed for a variety of conditions including fractures (open and closed), sprains, joint dislocations, crush injuries, ligament and muscle tears, tendon injuries, nerve injuries, congenital deformities and malformations, total joint or partial joint replacement, and cartilage injuries. Although many procedures are still performed through open surgical techniques, increasingly, numerous orthopedic procedures are being performed via an arthroscope inserted into the joint. Virtually any musculoskeletal (muscle, tendon, joint, bone, cartilage) injury, traumatic injury, or orthopedic surgical intervention can trigger a cascade that ultimately results in the formation of adhesions. In many instances, the adhesions must be broken down during repeat surgical interventions (e.g., capsulotomies, tendon releases, nerve entrapment releases, frozen joints, etc.). An adhesion barrier containing a fibrosis-inhibiting drug combination or individual component(s) thereof is best applied directly to the affected areas (as a solid, a film, a paste, a gel, a liquid or another such formulation) during open or arthroscopic orthopedic procedures. In a preferred embodiment, the barrier is sprayed under direct or arthrocopic vision onto the affected musculoskeletal tissue during the intervention. Since adhesions often occur in areas at a distance from the tissues actually instrumented during a surgical intervention, it is recommended that the barrier be applied to a wide area around the injured or repaired tissues. Preferred barriers include films, liquids, gels, pastes, sprays or other formulations that can be delivered during open procedures or through an endoscope, adhere to the tissues treated, and remain in place long enough to deliver the therapeutic agent and/or prevent adhesion formation. An ideal adhesion therapy will reduce the incidence, number and tenacity of adhesions and improve patient outcome by reducing pain, weakness and sensory abnormalities, preventing contractures, increasing range of motion, improving function, limiting physical deformity and disability, and reducing the need for repeat interventions.

As described above, the compositions for the prevention of surgical adhesions can be applied directly or indirectly to the tissue in an orthopedic surgery procedure. The anti-fibrosis drug combination (or individual component(s) thereof) or the composition that comprises the anti-fibrosis drug combination (or individual component(s) thereof) can be administered in any manner described herein. Exemplary methods include either direct application at the time of surgery or with arthroscopic, ultrasound, CT, MRI, or fluoroscopic guidance. If an implanted device is being placed, the composition for the prevention of adhesions can be applied to the surface of the implant, or to the surrounding tissues, in conjunction with placement of a medical device or implant at the surgical site. Representative examples of implants for use in orthopedic procedures include plates, rods, screws, pins, wires, total and partial joint prostheses (artificial hips, knees, shoulders, phalangeal joints), reinforcement patches, tissue fillers, synthetic bone fillers, bone cement, synthetic graft material, allograft material, autograft material, artificial discs, spinal cages, and intermedulary rods.

The anti-fibrosis drug combination (or individual component(s) thereof) or the composition that comprises the anti-fibrosis drug combination (or individual component(s) thereof) may be applied during open or arthroscopic orthopedic surgery: (a) to the tissue surface of the bone, joint, muscle, tendon, ligament, cartilage and any adjacent affected tissues (e.g., as an injectable, solution, paste, gel, in situ forming gel or mesh) during the surgical procedure; (b) to the surface of an implanted orthopedic device or implant and/or the tissue surrounding the implant (e.g., as an injectable, solution, paste, gel, in situ forming gel or mesh) before, during, or after the surgical procedure; (c) by intra-articular or endoscopic administration of the composition into the anatomical space (e.g., the joint space, tendon sheath, nerve root, spinal canal) at the surgical site (particularly useful for this embodiment is the use of injectable compositions containing polymeric carriers which release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where there is a risk of adhesion formation); (d) via percutaneous injection into the tissue as a solution as an infusate or as a sustained release preparation (intramuscular or intra-articular injection); (e) by guided catheter injection of the composition into the tissues and/or (f) by any combination of the aforementioned methods. Combination therapies (e.g., combinations with antithrombotic, anti-infective, and/or antiplatelet agents) can also be used in the manner described above.

In certain applications involving the placement of an orthopedic medical device or implant, it may be desirable to apply the anti-fibrosis drug combination (or individual component(s) thereof) or the composition comprising the anti-fibrosis drug combination (or individual component(s) thereof) at a site that is adjacent to an implant (preferably near the implant-tissue interface). This can be accomplished during open, endoscopic or catheter-based orthopedic procedures by applying the anti-fibrosis composition: (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the orthopedic implant; (c) to the surface of the implant and the tissue surrounding the implant (e.g., as an injectable, solution, paste, gel, in situ forming gel or mesh) before, during, or after implantation of the implant; (d) by topical application of the composition into the anatomical space (joint capsule, spinal canal, marrow, tendon sheath etc.) where the implant will be placed (particularly useful for this embodiment is the use of polymeric carriers which release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations which release the drug combination or individual component(s) thereof can be delivered into the region where the device will be inserted); (e) via percutaneous injection into the tissue surrounding the orthopedic implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

In certain embodiments, the anti-fibrosis composition may be delivered to the musculoskeletal tissue (or device/tissue interface) in the form of a spray or gel during open, endoscopic or catheter-based procedures. The fibrosis-inhibiting drug combination (or individual component(s) thereof) can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting drug combination or individual component(s) thereof may be incorporated directly into either the 4 armed NHS-derivatized polyethylene glycol, the acidic solution or the basic buffer. In another embodiment, the fibrosis-inhibiting drug combination or individual component(s) thereof may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, 8-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially, and the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The fibrosis-inhibiting drug combination or individual component(s) thereof may be incorporated directly into either the 4 armed NHS-derivatized polyethylene glycol, or the basic buffer. In another embodiment, the fibrosis-inhibiting drug combination or individual component(s) thereof may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially, and the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

vi) Adhesion Prevention in Reconstructive and Cosmetic Procedures

In certain embodiments, adhesions may be associated with a cosmetic or reconstructive surgical procedure. The use of soft tissue implants for cosmetic applications (aesthetic and reconstructive) is common in breast augmentation, breast reconstruction after cancer surgery, craniofacial procedures, reconstruction after trauma, congenital craniofacial reconstruction and oculoplastic surgical procedures to name a few.

The clinical function of a soft tissue implant depends upon the implant being able to effectively maintain its shape over time. In many instances, when these devices are implanted in the body, they are subject to a “foreign body” response from the surrounding host tissues. The body recognizes the implanted device as foreign, which triggers an inflammatory response followed by encapsulation of the implant with fibrous connective tissue (adhesion formation). Encapsulation of surgical implants complicates a variety of reconstructive and cosmetic surgeries, but is particularly problematic in the case of breast reconstruction surgery where the breast implant becomes surrounded by a fibrous capsule that alters anatomy and function. Scar capsules that harden and contract (known as “capsular contractures”) are the most common complication of breast implant or reconstructive surgery. Capsular (fibrous) contractures can result in hardening of the breast, loss of the normal anatomy and contour of the breast, discomfort, weakening and rupture of the implant shell, asymmetry, infection, and patient dissatisfaction. Further, fibrous encapsulation of any soft tissue implant can occur even after a successful implantation if the device is manipulated or irritated by the daily activities of the patient. Bleeding in and around the implant can also trigger a biological cascade that ultimately leads to excess scar tissue formation. Furthermore, certain types of implantable prostheses (such as breast implants) include gel fillers (e.g., silicone) that tend to leak through the membrane envelope of the implant and can potentially cause a chronic inflammatory response in the surrounding tissue (which encourages tissue encapsulation and contracture formation). The effects of unwanted scarring in the vicinity of the implant are the leading cause of additional surgeries to correct defects, break down scar tissue (capsulotomy or capsulaectomy), to replace the implant, or remove the implant. The local administration of anti-adhesive compositions, alone or loaded with a fibrosis-inhibiting agent, can be utilized in a wide array of cosmetic and reconstructive procedures to improve patient outcomes.

Soft tissue implants are used in a variety of cosmetic, plastic, and reconstructive surgical procedures and may be delivered to many different parts of the body, including, without limitation, the face, nose, breast, chin, buttocks, chest, lip and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Of all soft tissue implantation procedures, breast implant placement for augmentation or breast reconstruction after mastectomy is the most frequently performed cosmetic surgery implant procedure. For example, in 2002 alone, over 300,000 women had breast implant surgery. Of these, approximately 80,000 were breast reconstructions following a mastectomy due to cancer.

The process for failure of all soft tissue implants is similar regardless of anatomical placement. However, since breast implants have been the most widely studied soft tissue implant, they will be used to illustrate the present invention. In general, breast augmentation or reconstructive surgery involves the placement of a commercially available breast implant, consisting of a capsule filled with either saline or silicone, into the tissues underneath the mammary gland. Four different incision sites have historically been used for breast implantation: axillary (armpit), periareolar (around the underside of the nipple), inframamary (at the base of the breast where it meets the chest wall) and transumbilical (around the belly button). The tissue is dissected away through the small incision, often with the aid of an endoscope (particularly for axillary and transumbilical procedures where tunneling from the incision site to the breast is required). A pocket for placement of the breast implant is created in either the subglandular or the subpectorial region. For subglandular implants, the tissue is dissected to create a space between the glandular tissue and the pectoralis major muscle that extends down to the inframammary crease. For subpectoral implants, the fibers of the pectoralis major muscle are carefully dissected to create a space beneath the pectoralis major muscle and superficial to the rib cage. Careful hemostasis is essential (since it can contribute to complications such as capsular contractures), so much so that minimally invasive procedures (axillary, transumbilical approaches) must be converted to more open procedures (such as periareolar) if bleeding control is inadequate. Depending upon the type of surgical approach selected, the breast implant is often deflated and rolled up for placement in the patient. After accurate positioning is achieved, the implant can then be filled or expanded to the desired size.

Although many patients are satisfied with the initial procedure, significant percentages suffer from complications that frequently require a repeat intervention to correct. Encapsulation of a breast prosthesis that creates a periprosthetic shell (called capsular contracture) is the most common complication reported after breast enlargement, with up to 50% of patients reporting some dissatisfaction. Calcification can occur within the fibrous capsule adding to its firmness and complicating the interpretation of mammograms. Multiple causes of capsular contracture have identified including: foreign body reaction, migration of silicone gel molecules across the capsule and into the tissue, autoimmune disorders, genetic predisposition, infection, hematoma, and the surface characteristics of the prosthesis. Although no specific etiology has been repeatedly identified, at the cellular level, abnormal fibroblast activity stimulated by a foreign body is a consistent finding. Periprosthetic capsular tissues contain macrophages and occasional T- and B-lymphocytes, suggesting an inflammatory component to the process. Implant surfaces have been made both smooth and textured in an attempt to reduce encapsulation, however, neither has been proven to produce consistently superior results. Animal models suggest that there is an increased tendency for increased capsular thickness and contracture with textured surfaces that encourage fibrous tissue ingrowth on the surface. Placement of the implant in the subpectoral location appears to decrease the rate of encapsulation in both smooth and textured implants.

From a patient's perspective, the biological processes described above lead to a series of commonly described complaints. Implant malposition, hardness and unfavorable shape are the most frequently sited complications and are most often attributed to capsular contracture. When the surrounding scar capsule begins to harden and contract, it results in discomfort, weakening of the shell, asymmetry, skin dimpling and malpositioning. True capsular contractures will occur in approximately 10% of patients after augmentation, and in 25% to 30% of reconstruction cases, with most patients reporting dissatisfaction with the asthetic outcome. Scarring leading to asymmetries occurs in 10% of augmentations and 30% of reconstructions and is the leading cause of revision surgery. Skin wrinkling (due to the contracture pulling the skin in towards the implant) is a complication reported by 10% to 20% of patients. Scarring has even been implicated in implant deflation (1-6% of patients; saline leaking out of the implant and “deflating” it), when fibrous tissue ingrowth into the diaphragmatic valve (the access site used to inflate the implant) causes it to become incontinent and leak. In addition, over 15% of patients undergoing augmentation will suffer from chronic pain and many of these cases are ultimately attributable to scar tissue formation. Other complications of breast augmentation surgery include late leaks, hematoma (approximately 1-6% of patients), seroma (2.5%), hypertrophic scarring (2-5%) and infections (about 1-4% of cases).

Correction can involve several options including removal of the implant, capsulotomy (cutting or surgically releasing the capsule), capsulectomy (surgical removal of the fibrous capsule), or placing the implant in a different location (i.e., from subglandular to subpectoral). Ultimately, additional surgery (revisions, capsulotomy, removal, re-implantation) is required in over 20% of augmentation patients and in over 40% of reconstruction patients, with scar formation and capsular contracture being far and away the most common cause. Procedures to break down the scar may not be sufficient, and approximately 8% of augmentations and 25% of reconstructions ultimately have the implant surgically removed.

A fibrosis-inhibiting drug combination or individual component(s) thereof or composition comprising the drug combination or individual component(s) delivered locally from the soft tissue implant or administered locally into the tissue surrounding the soft tissue implant can minimize fibrous tissue formation, encapsulation and capsular contracture. Application of a fibrosis-inhibiting composition onto the surface of a soft tissue implant or incorporated into a soft tissue implant (e.g., the drug combination or individual component(s) thereof is incorporated into the saline, gel or silicone within the implant and passively diffuses across the capsule into the surrounding tissue) may minimize or prevent fibrous contracture. Infiltration of a fibrosis-inhibiting drug combination, individual component(s) thereof, or composition into the tissue surrounding the soft tissue implant, or into the surgical pocket where the implant will be placed, is another strategy for preventing the formation of scar and capsular contracture in augmentation and reconstructive surgery.

As described previously, adhesions and fibrous encapsulation of cosmetic implants is a common complication of asthetic and reconstructive surgery. A variety of commercially available adhesion barriers are suitable for combining with a fibrosis-inhibiting drug combination or individual component(s) thereof in the management of this complication. Commercially available materials that may be loaded with a fibrosis-inhibiting drug combination or individual component(s) thereof and applied to the surface of a soft tissue implant, contained within the “filler” (typically saline, silicone or gel) of a soft tissue implant, or infiltrated into the tissue surrounding the implantation site for the prevention of adhesions in cosmetic surgery include: (a) sprayable collagen-containing formulations such as COSTASIS or CT3; (b) sprayable PEG-containing formulations such as COSEAL, ADHIBIT, FOCALSEAL, SPRAYGEL or DURASEAL; (c) fibrinogen-containing formulations such as FLOSEAL or TISSEAL; (d) hyaluronic acid-containing formulations such as RESTYLANE or PERLANE, HYLAFORM, SYNVISC, SEPRAFILM or SEPRACOAT; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (g) dextran sulfate gels such as the ADCON series of gels; and (h) lipid based compositions such as ADSURF. Several of the above agents (e.g., formulations containing PEG, collagen, or fibrinogen such as COSEAL, CT3, ADHIBIT, COSTASIS, FOCALSEAL, SPRAYGEL, DURASEAL, TISSEAL AND FLOSEAL) have the added benefit of being hemostats and vascular sealants, which given the suspected role of inadequate hemostasis in the development of capsular contracture, should also be of benefit in the practice of this invention. It should be obvious to one of skill in the art that commercial compositions not specifically cited above as well as next-generation and/or subsequently-developed commercial products are to be anticipated and are suitable for use under the present invention.

As described above, the compositions for the prevention of surgical adhesions can be applied directly or indirectly to the tissue around the cosmetic implant site. The anti-fibrosis drug combination (or individual component(s) thereof) or the composition that comprises the anti-fibrosis drug combination (or individual component(s) thereof) can be administered in any manner described herein. Exemplary methods include either direct application at the time of surgery or with endoscopic, ultrasound, CT, MRI, or fluoroscopic guidance and in conjunction with placement of a cosmetic implant at the surgical site. Representative examples of implants for use in cosmetic procedures include, without limitation, saline breast implants, silicone breast implants, chin and mandibular implants, nasal implants, cheek implants, lip implants, other facial implants, pectoral and chest implants, malar and submalar implants, tissue fillers, and buttocks implants.

The anti-fibrosis drug combination (or individual component(s) thereof) or the composition comprising the anti-fibrosis drug combination (or individual component(s) thereof) may be applied during open or endoscopic cosmetic surgery: (a) to the soft tissue implant surface (e.g., as an injectable, solution, paste, gel, in situ forming gel, or mesh) before, during, or after the implantation procedure; (b) to the surface of the tissue (e.g., as an injectable, solution, paste, gel, in situ forming gel or mesh) of the implantation pocket immediately prior to, or during implantation of the soft tissue implant; (c) to the surface of the soft tissue implant and/or the tissue surrounding the implant (e.g., as an injectable, solution, paste, gel, in situ forming gel or mesh) before, during, or after implantation of the soft tissue implant; (d) by topical application of the anti-fibrosis drug combination (or individual component(s) thereof) into the anatomical space where the soft tissue implant will be placed (particularly useful for this embodiment is the use of polymeric carriers which release the fibrosis-inhibiting drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) and can be delivered into the region where the implant will be inserted); (e) via percutaneous injection into the tissue surrounding the implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

A composition that includes an anti-scarring drug combination or individual component(s) thereof can be infiltrated into the space (surgically created pocket) where the soft tissue implant will be implanted. In certain applications involving the placement of a cosmetic soft tissue implant, it may be desirable to apply the anti-fibrosis composition at a site that is adjacent to an implant (preferably near the implant-tissue interface). This can be accomplished during open, endoscopic or catheter-based cosmetic procedures by applying the anti-fibrosis drug combination (or individual component(s) thereof) or the composition that comprises the anti-fibrosis drug combination (or individual component(s) thereof): (a) to the implant surface (e.g., as an injectable, solution, paste, gel, in situ forming gel, or mesh) before, during, or after the implantation procedure; (b) to the surface of the adjacent tissue (e.g., as an injectable, solution, paste, gel, in situ forming gel, or mesh) immediately prior to, during, or after implantation of the soft tissue implant; (c) to the surface of the soft tissue implant and the tissue surrounding the implant (e.g., as an injectable, solution, paste, gel, in situ forming gel or mesh) before, during, or after implantation of the implant; (d) by topical application of the composition into the anatomical space (surgical pocket; for example, in breast implants this is the subglandular or subpectoral space) where the soft tissue implant will be placed (particularly useful for this embodiment is the use of polymeric carriers which release the fibrosis-inhibiting drug combination or individual component(s) thereof over a period ranging from several hours to several weeks—fluids, suspensions, emulsions, microemulsions, microspheres, pastes, gels, microparticulates, sprays, aerosols, solid implants and other formulations which release the drug combination or individual component(s) thereof can be delivered into the region where the device will be inserted); (e) via percutaneous injection into the tissue surrounding the soft tissue implant as a solution, as an infusate, or as a sustained release preparation; and/or (f) by any combination of the aforementioned methods. Combination therapies (e.g., combinations with antithrombotic, anti-infective, and/or antiplatelet agents) can also be used.

In certain embodiments, the anti-fibrosis drug combination (or individual component(s) thereof) or the composition that comprises drug combination (or individual component(s) thereof) may be delivered to the soft tissue implant (or implant/tissue interface) in the form of a spray or gel during open, endoscopic or catheter-based procedures. The fibrosis-inhibiting drug combination or individual component(s) thereof can be incorporated directly into the surgical adhesion barrier or it can be incorporated into a secondary carrier (polymeric or non-polymeric), as described above, that is then incorporated into the adhesion barrier. Examples of polymer compositions that may be in the form of a spray or gel include poly(ethylene glycol)-based systems, fibrinogen-containing systems, hyaluronic acid and crosslinked hyaluronic acid compositions. These compositions can be applied as the final composition or they can be applied as materials that form a crosslinked gel in situ.

In another aspect, an activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means. Following application of the composition to the surgical site, any excess solution may be removed from the surgical site if deemed necessary. At this point in time, the surgical site can be closed using conventional means (e.g., sutures, staples, or a bioadhesive). In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH>about 8). The fibrosis-inhibiting drug combination or individual component(s) thereof may be incorporated directly into either the 4 armed NHS-derivatized polyethylene glycol, the acidic solution or the basic buffer. In another embodiment, the fibrosis-inhibiting drug combination or individual component(s) thereof may be incorporated into a secondary carrier that may then be incorporated into the 4 armed NHS-derivatized polyethylene glycol, the acidic solution and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In yet another aspect, an activated polymer can be applied to the surgical site in the solid state. The activated polymer can react with the tissue surface to which it was applied as the polymer hydrates. A biologically acceptable buffer, with a pH greater than 7.5 can be applied to the tissue before and/or after the solid activated polymer has been applied. In one embodiment, the activated polymer can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form. The antifibrosisfibrosis-inhibiting agent(s) may be incorporated directly into either the 4 armed NHS-derivatized polyethylene glycol, or the basic buffer. In another embodiment, the fibrosis-inhibiting agent may be incorporated into a secondary carrier that may then be incorporated into the 4 armed NHS-derivatized polyethylene glycol, and/or the basic buffer. The secondary carriers may include microparticles and/or microspheres which are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the tissue reactive polymer may be applied initially and then the fibrosis-inhibiting drug combination or individual component(s) thereof may then be applied to the coated tissue. The fibrosis-inhibiting drug combination or individual component(s) thereof may be applied directly to the tissue or it may be incorporated into a secondary carrier. The secondary carriers may include microspheres (as described above), microparticles (as described above), gels (e.g., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

vii) Agents and Dosages of Fibrosis-Inhibitors

In certain aspects of the invention, compositions are provided that can release a therapeutic agent able to reduce scarring (i.e., a fibrosis-inhibiting drug combination or individual component(s) thereof) at a surgical site. Within one embodiment of the invention, surgical adhesion barriers may include or be adapted to release an agent that inhibits one or more of the five general components of the process of fibrosis (or scarring), including: inflammatory response and inflammation, migration and proliferation of connective tissue cells (such as fibroblasts or smooth muscle cells), formation of new blood vessels (angiogenesis), deposition of extracellular matrix (ECM), and remodeling (maturation and organization of the fibrous tissue). By inhibiting one or more of the components of fibrosis (or scarring), the overgrowth of scar tissue may be inhibited or reduced.

Examples of fibrosis-inhibiting drug combinations for use in surgical adhesion barriers include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for surgical adhesion prevention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring drug combination or individual component(s) thereof is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In certain embodiments, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing drug combinations or individual components thereof should be administered under the following dosing guidelines. The total amount (dose) of anti-scarring agent(s) in the drug combinations or compositions that comprise the drug combinations can be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of surface to which the agent is applied may be in the range of about 0.01 μg/mm²-1 μg/mm², or 1 μg/mm²-1 μg/mm2, or 10 μg/mm²-250 μg/mm², 250 μg/mm²-1000 μg/mm², or 1000 μg/mm²-2500 μg/mm².

Provided below are exemplary dosage ranges for various anti-scarring drug combinations or individual components thereof that can be used for treating or preventing surgical adhesions in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

According to another aspect, any anti-infective agent described above may be used in combination with the present compositions for surgical adhesion prevention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintaine on the tissue surface.

(b) Inflammatory Arthritis

In certain embodiments, the present invention provides compositions for the treatment and prevention of inflammatory arthritis. The compositions of the present invention can comprise anti-fibrosis drug combinations or optionally additional components (e.g., secondary agents, or polymers).

Inflammatory arthritis is a serious health problem in developed countries, particularly given the increasing number of aged individuals and includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behcet's syndrome, sarcoidosis, and osteoarthritis—all of which feature inflamed and/or painful joints as a prominent symptom.

In one aspect, the present compositions may be used to treat or prevent osteoarthritis (OA). Osteoarthritis is a common, debilitating, costly, and currently incurable disease. The disease is characterized by abnormal functioning of chondrocytes and their terminal differentiation, leading ultimately to the initiation of OA and the breakdown of the cartilage matrix in the articular cartilage of affected joints. Age is the most powerful risk factor for OA, but major joint trauma, excessive weight, and repetitive joint use are also important risk factors for OA. The pattern of joint involvement in OA is also influenced by prior vocational or avocational overload.

OA can be of primary (idiopathic) and secondary types. Primary OA is most commonly related to age. Repetitive use of the joints, particularly the weight-bearing joints such as hips, knees, feet and back, irritates and inflames the joints and causes joint pain and swelling. Eventually, cartilage begins to degenerate by flaking or forming tiny crevasses. In advanced cases, there is a total loss of the cartilage cushion between the bones of the joints. Loss of the cartilage cushion causes friction between the bones, leading to pain and limitation of joint mobility. Inflammation of the cartilage can also stimulate new bone outgrowths (spurs) to form around the joints.

Secondary OA is pathologically indistinguishable from idiopathic OA but is attributable to another disease or condition. Conditions that can lead to secondary OA include obesity, repeated trauma (e.g., ligament tears, cartilage tears), surgery to the joint structures (ligament repairs, menisectomy, cartilage removal), abnormal joints at birth (congenital abnormalities), gout, diabetes, and other metabolic disorders.

In one aspect, the present compositions may be used to treat or prevent rheumatoid arthritis (RA). Rheumatoid arthritis is a multisystem chronic, relapsing, inflammatory disease of unknown cause. Although many organs can be affected, RA is basically a severe form of chronic synovitis that sometimes leads to destruction and ankylosis of affected joints (Robbins Pathological Basis of Disease, by R. S. Cotran, V. Kumar, and S. L. Robbins, W. B. Saunders Co., 1989). Pathologically the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and eventually the pannus grows to fill the joint space. The pannus develops an extensive network of new blood vessels through the process of angiogenesis which is essential to the evolution of the synovitis. Digestive enzymes (matrix metalloproteinases such as collagenase and stromelysin) and other mediators of the inflammatory process (e.g., hydrogen peroxide, superoxides, lysosomal enzymes, and products of arachadonic acid metabolism) released from the cells of the pannus tissue break down the cartilage matrix and cause progressive destruction of the cartilage. The pannus invades the articular cartilage leading to erosions and fragmentation of the cartilage tissue. Eventually there is erosion of the subchondral bone with fibrous ankylosis and ultimately bony ankylosis, of the involved joint.

It is generally believed, but not conclusively proven, that RA is an autoimmune disease, and that many different arthrogenic stimuli activate the immune response in the immunogenetically susceptible host. Both exogenous infectious agents (Ebstein-Barr virus, rubella virus, cytomegalovirus, herpes virus, human T-cell lymphotropic virus, mycoplasma, and others) and endogenous proteins (collagen, proteoglycans, altered immunoglobulins) have been implicated as the causative agent which triggers an inappropriate host immune response. Regardless of the inciting agent, autoimmunity plays a role in the progression of the disease. In particular, the relevant antigen is ingested by antigen-presenting cells (macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive, inappropriate immune response directed against the host tissues (e.g., antibodies directed against type II collagen, antibodies directed against the Fc portion of autologous IgG (called “Rheumatoid Factor”)). This further amplifies the immune response and hastens the destruction of the cartilage tissue. Once this cascade is initiated, numerous mediators of cartilage destruction are responsible for the progression of rheumatoid arthritis.

In rheumatoid arthritis, articular cartilage is destroyed when it is invaded by pannus tissue (which is composed of inflammatory cells, blood vessels, and connective tissue). Generally, chronic inflammation in itself is insufficient to result in damage to the joint surface, but a permanent deficit is created once fibrovascular tissue digests the cartilage tissue. The abnormal growth of blood vessels and pannus tissue may be inhibited by treatment with fibrosis-inhibiting compositions. Incorporation of an anti-scarring drug combination or individual component(s) thereof into these compositions or other intra-articular formulations, can provide an approach that can reduce the rate of progression of the disease.

Thus, within one aspect of the present invention, methods are provided for treating or preventing inflammatory arthritis comprising the step of administering to a patient in need thereof a therapeutically effective amount of an anti-scarring drug combination or a composition comprising the anti-scarring drug combination. Inflammatory arthritis includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behcet's syndrome, sarcoidosis, and osteoarthritis—all of which feature inflamed and/or painful joints as a prominent symptom.

An effective anti-scarring therapy for inflammatory arthritis will accomplish one or more of the following: (i) decrease the severity of symptoms (pain, swelling and tenderness of affected joints; morning stiffness, weakness, fatigue, anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/attenuate chronic progression of the disease.

According to the present invention, any anti-scarring drug combination described above could be utilized in the practice of this invention. Within certain embodiments of the invention, the composition may release an agent that inhibits one or more of the general components of the process of fibrosis (or scarring) associated with inflammatory arthritis, including: (a) formation of new blood vessels (angiogenesis), (b) migration and/or proliferation of connective tissue cells (such as fibroblasts or synoviocytes), (c) destruction of the cartilage matrix by metalloproteinase activity, (d) inflammatory response by cytokines (such as IL-1, TNFα, FGF, VEGF). By inhibiting one or more of the components of fibrosis (or scarring), cartilage loss may be inhibited or reduced.

In one aspect, the composition includes an anti-scarring drug combination or individual component(s) thereof and a polymeric carrier suitable for application to treat inflammatory arthritis. Numerous polymeric and non-polymeric delivery systems and compositions containing an anti-scarring drug combination (or individual component(s) thereof) for use in the treatment of inflammatory arthritis have been described above. An anti-scarring drug combination or individual component(s) thereof may be administered systemically (orally, intravenously, or by intramuscular or subcutaneous injection) in the minimum dose to achieve the above mentioned results. For patients with only a small number of joints affected, or with disease more prominent in a limited number of joints, the anti-scarring drug combination or individual component(s) thereof can be directly injected into the affected joint (intra-articular injection) via percutaneous needle insertion into the joint capsule, or as part of an arthroscopic procedure performed on the joint. In a preferred embodiment, the intra-articular formulation containing a fibrosis-inhibiting drug combination or individual component(s) thereof is administered to a joint following an injury with a high probability of inducing subsequent arthritis (e.g., cruciate ligament tears in the knee, meniscal tears in the knee). The drug combination or individual component(s) thereof is administered for a period sufficient (either through sustained release preparations and/or repeated injections) to protect the cartilage from breakdown as a result of the injury (or the surgical procedure used to treat it).

The anti-scarring drug combination or individual component(s) thereof can be administered in any manner described herein. However, preferred methods of administration include intravenous, oral, subcutaneous injection, or intramuscular injection. A particularly preferred embodiment involves the administration of the fibrosis-inhibiting drug combination or individual component(s) thereof as an intra-articular injection (directly, via arthroscopic or radiologic guidance, or irrigated into the joint as part of an open surgical procedure). The anti-scarring drug combination or individual component(s) thereof can be administered as a chronic low dose therapy to prevent disease progression, prolong disease remission, or decrease symptoms in active disease. Alternatively, the therapeutic drug combination or individual component(s) thereof can be administered in higher doses as a “pulse” therapy to induce remission in acutely active disease; such as the acute inflammation that follows a traumatic joint injury (intra-articular fractures, ligament tears, meniscal tears, as described below). The minimum dose capable of achieving these endpoints can be used and can vary according to patient, severity of disease, formulation of the administered agent, potency and/or tolerability of the drug combination or individual component(s) thereof, clearance of the drug combination or individual component(s) thereof from the joint, and route of administration.

In one preferred embodiment, the fibrosis-inhibiting drug combination (or individual component(s) thereof) or the composition comprising the fibrosis-inhibiting drug combination can be an intra-articular injectable hyaluronic acid-based composition. Hyaluronic acid, which is a normal element of joint synovial fluid, lubricates the joint surface during normal activities (resting, walking) and helps prevent mechanical damage and decrease shock on the joint in high impact activities (such as running, jumping). In patients with OA, the elasticity and viscosity of the synovial fluid and the synovial hyaluronic acid concentration are reduced. It is believed that this contributes to the breakdown of the articular cartilage within the joint. Intra-articularly administered HA (typically sodium hyaluronate) penetrates the articular cartilage surface, the synovial tissue, and the capsule of the joint for a period of time after injection. By injecting hyaluronic acid into the joint (known as visco-supplementation), it is possible to partially restore the normal environment of the synovial fluid, reduce pain, and potentially prevent further damage and disability. Representative examples of hyaluronic acid compositions used in visco-supplementation are described in U.S. Pat. Nos. 6,654,120, 6,645,945, and 6,635,287. As such, HA-containing materials are administered as an intra-articular injection (as either a single treatment or a course of repeated treatment cycles) for the treatment of painful osteoarthritis of the knee in patients who have insufficient pain relief from conservative therapies. Occasionally other joints such as hips (injected under fluoroscopy), ankles, shoulders and elbow joints, are also injected with HA to relieve the symptoms of the disease in those particular joints. Depending upon the particular commercial product, the HA material is injected into the joint once a week for 5 to 6 consecutive weeks. When effective, patients may report that they receive symptomatic relief for a period of 6 months or more—at which time the cycle may be repeated to prolong the activity of the therapy. Despite the sustained benefit in some patients, the injected HA is rapidly cleared (removed) from the joint by the body over a period of several days. Prolonging the residence time of the HA in the joint by inhibiting its breakdown may be expected to enhance its efficacy and increase the duration of symptomatic relief. By adding a fibrosis-inhibiting drug combination or individual component(s) thereof to the HA, the intra-articular injection has the added benefit of helping to prevent cartilage breakdown (i.e., it is “chondroprotective”).

A variety of commercially available HA compositions for the treatment of inflammatory arthritis may be combined with one or more agents according to the present invention including: SYNVISC (Biomatrix, Inc., Ridgefield, N.J.)—an elastoviscous fluid containing hylan (a derivative of sodium hyaluronate (hyaluronan)) polymers derived from rooster combs, HYALGAN (Sanofi-Synthelabo Inc. New York, N.Y.), and ORTHOVISC (Ortho Biotech Products, Bridgewater, N.J.)—a highly purified, high molecular weight, high viscosity injectable form of HA intended to relieve pain and to improve joint mobility and range of motion in patients suffering from osteoarthritis (OA) of the knee. ORTHOVISC is injected into the knee to restore the elasticity and viscosity of the synovial fluid. HYVISC is a high molecular weight, injectable HA product developed by Anika Therapeutics (Woburn, Mass.) currently being used to treat osteoarthritis and lameness in racehorses. Other HA-based viscosupplementation products for the treatment of osteoarthritis include SUPARTZ from Seikagaku Corp. (Japan), SUPLASYN from Bioniche Life Sciences, Inc. (Canada), ARTHREASE from DePuy Orthopaedics, Inc. (Warsaw, Ind.), and DUROLANE from Q-Med AB (Sweden).

In one aspect, the compositions of the present invention may be used for the management of osteoarthritis in animals (e.g., horses). It should be noted that some HA products (notably HYVISC by Boehringer Ingelheim Vetmedica, St. Joseph, Mo.) are used in veterinary applications (typically in horses to treat osteoarthritis and lameness).

Other intra-articular compositions used to treat arthritis include corticosteroids. The most common corticosteroids currently used for inflammatory arthritis are methylprednisolone acetate (DEPO-MEDROL, Pharmacia & Upjohn Company, Kalamazoo, Mich.), and triacinolone acetonide (KENALOG, Bristol-Myers Squibb, New York, N.Y.). By adding a fibrosis-inhibiting drug combination or individual component(s) thereof to the intra-articular corticosteroid injection, the intra-articular injection has the added benefit of helping to prevent cartilage breakdown (i.e., it is “chondroprotective”).

Formulations that can be used in these applications include solutions, topical formulations (e.g., solution, cream, ointment, gel) emulsions, micellar solutions, gels (crosslinked and non-crosslinked), suspensions and/or pastes. One form of the formulation is as an injectable composition. For compositions that further contain a polymer to increase the viscosity of the formulation, hyaluronic acid (crosslinked, derivatized and/or non-crosslinked) is an exemplary material. These formulations can further comprise additional polymers (e.g., collagen, poly(ethylene glycol) or dextran) as well as biocompatible solvents (e.g., ethanol, DMSO, or NMP). In one embodiment, the fibrosis-inhibiting therapeutic drug combination or individual component(s) thereof can be incorporated directly into the formulation. In another embodiment, the fibrosis-inhibiting therapeutic drug combination or individual component(s) thereof can be incorporated into a secondary carrier (e.g., micelles, liposomes, emulsions, microspheres, nanospheres etc, as described above). The microsphere and nanospheres may be comprised of degradable polymers. Degradable polymers that can be used include poly(hydroxyl esters) (e.g., PLGA, PLA, PCL, and the like), as well as polyanhydrides, polyorthoesters and polysaccharides (e.g., chitosan and alginates).

In one embodiment, the fibrosis-inhibiting drug combination or individual component(s) thereof further comprises a polymer where the polymer is a degradable polymer. The degradable polymers may include polyesters where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the fibrosis-inhibiting agent/composition may further comprise a solvent, a liquid oligomer or liquid polymer such that the final composition may be passed through a 18G needle. The reagents that may be used include ethanol, NMP, PEG 200, PEG 300 and low molecular weight liquid polymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator.

In another embodiment, the fibrosis-inhibiting drug combination or individual component(s) thereof may be in the form of a solution or suspension in an organic solvent, a liquid oligomer or a liquid polymer. In this embodiment, reagents such as ethanol, NMP, PEG 200, PEG 300 and low molecular weight liquid polymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, may be used.

Examples of fibrosis-inhibiting drug combinations for use in the treatment of inflammatory arthritis include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for the treatment of inflammatory arthritis will depend on a variety of factors, including the type of formulation and treatment site. However, certain principles can be applied in the application of this art. Drug dose can be calculated as a function of dose per unit area (of the treatment site), total drug dose administered can be measured and appropriate surface concentrations of active drug can be determined. For local application, drugs are to be used at concentrations that range from several times more than to 50%, 20%, 10%, 5%, or even less than 1% of the concentration typically used in a single systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing drug combination or individual component(s) thereof should be administered under the following dosing guidelines. The total amount (dose) of anti-scarring agent(s) in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations for the treatment of inflammatory arthritis in accordance with the invention. The following dosages are particularly useful for intra-articular administration:

Exemplary anti-fibrotic drug combinations for dose explanation purposes include, but are not limited to, amoxapine and prednisolon&, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

According to another aspect, any anti-infective agent described above may be used in conjunction with compositions for the treatment of inflammatory arthritis. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

(c) Prevention of Cartilage Loss (“Chondroprotection”)

In another aspect, anti-fibrosis compositions can be used to prevent or reduce the loss of cartilage loss following an injury (e.g., cruciate ligament tear and/or meniscal tear). It has been known for a long time that damage to a joint can predispose a patient to develop osteoarthritis in the joint at a subsequent point in time, but there has been no effective treatment to prevent this occurrence. Instead most of the focus from the medical community and researchers has been on the treatment of the arthritis after it has become established. Treatments for established disease include anti-inflammatory drugs (non-steroidal and steroidal), lubricants or synovial fluid replacements, surgery and joint replacement for severe disease.

Trauma to a joint can take many forms, ranging from a simple sprain which can heal spontaneously to a fracture that creates so many bone fragments that it is almost impossible to reconstruct the joint. The focus for treatment of these injuries revolves around restoring the joint to its normal anatomical state and to resume regular motion. Risk factors for developing arthritis are related to the extent of trauma, the extent of the joint disruption, the degree of the fracture or dislocations, whether or not it is a weight bearing joint, and the characteristic of the joint itself. In general, the greater the trauma to the joint, the greater the risk that the patient will develop osteoarthritis later in life. Surgical correction of a joint to its pre-injury anatomy does not guarantee the prevention of arthritis. In the case of an intra-articular fracture, for example a plateau fracture of the tibia, the treatment is to surgically reconstruct the joint so that it reverts back to a congruent, smooth and intact joint surface with no “step defects” or pieces out of place that would interfere with the gliding of the femur on its surface. Despite improved surgical techniques in repairing these fractures, patients with such fractures have a very high probability of developing degenerative arthritis later on in life.

Anterior cruciate ligament (ACL) injuries in the knee represent a classic example of an injury that predisposes patients to potentially severe degenerative arthritis. The ACL is the ligament that joins the anterior tibial plateau to the posterior femoral intercondylar notch. It is composed of multiple non-parallel fibers with variable fiber lengths that function in bundles to provide tension and mechanical stability to the knee throughout its range of motion. The ACL's stabilizing role has four main functions, including (a) restraining anterior translation of the tibia; (b) preventing hyperextension of the knee; (c) acting as a secondary stabilizer to the valgus stress, reinforcing the medial collateral ligament; and (d) controlling rotation of the tibia on the femur during femoral extensions, and thus, controlling movements such as side-stepping and pivoting. Generally, ACL deficiency results in subluxation of the tibia on the femur causing stretching of the enveloping capsular ligaments and abnormal shear forces on the menisci and on the articular cartilage. Delay in diagnosis and treatment gives rise to increased intra-articular damage as well as stretching of the secondary stabilizing capsular structures.

Despite the known high risk for developing osteoarthritis, patients generally have no associated fractures and have normal x-rays at the time of presentation post-ACL injury. Yet it is well documented that anyone who suffers an ACL injury has a high probability of developing arthritis: 50% by 10 years and 80% by 20 years post-injury. Generally after an ACL rupture patients suffer from instability since the ligament is critical in stabilizing the joint during pivoting and rotation. For example, it is not only required for demanding pivoting sports such as basketball, it is also required for daily activity such as a mother holding her baby as she pivots to get an item from the fridge.

The typical treatment and management of an ACL tear is reconstruction using a graft to replace the torn ACL. The graft may be taken from elsewhere in the patient's extremity (autograft), harvested from a cadaver (allograft) or may be made from a synthetic material. Autograft is the most widely performed orthopedic ACL reconstruction. The technique involves harvesting the patient's own tissue, which may be the mid-third of the patellar tendon with bone attached at both ends, one or two medial hamstrings, or the quadriceps tendon with bone at one end. Synthetic materials have the advantage of being readily available, however, there is a higher failure rate of synthetic grafts compared to autografts and allografts and they have mechanical properties that do not closely resemble the normal ligament. Successful ACL reconstruction is dependent on a number of factors, including surgical technique, post-operative rehabilitation and associated secondary ligament instability. During the surgical procedure, arthroscopy is used to determine whether there are any other associated injuries, which may be treated at the same time, such as meniscal tears or chondral trauma. The surgical procedure is done through a small accessory incision, whereby a tunnel is drilled through the tibia and femur so that the graft may be inserted and fixed.

Surgical reconstruction was initially thought to provide a permanent solution: re-establish a stable knee and prevent degeneration. But other studies demonstrated that after joint injury, there is a cascade of inflammatory activity that once initiated, can be destructive to the joint. This explains why surgical repair itself would have not impact on the prevention of degeneration in traumatized joints; stabilizing a joint or the macro reconstruction of a joint does not address the fundamental underlying biology. Unfortunately, although long-term data has shown that surgery is indeed successful in stabilizing the knee and getting people back to normal activity; it has no impact on the subsequent rate of development of osteoarthritis. As a result, the standard of care to day is to repair the joint acutely and treat the arthritis when it ultimately develops. It should be noted that all joints (in addition to knees) have the potential to become arthritic after trauma, but joints typically involved include; fingers, thumbs, metacarpal (wrist), elbow, shoulder, spine joints (facets, sacro-iliac), temperomandibular, otic bones, hips, ankles, tarsal and toes, especially the hallux.

Fibrosis-inhibiting drug combinations such as amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate may demonstrate in animal experiments an ability to prevent cartilage breakdown following cruciate ligament tears. This effect has been seen with antifibrotic agents both in an inflammatory model and biomechanical model of joint injury. Hartley Guinea pigs subjected to surgical transaction of the anterior cruciate ligament represent a mechanical model for arthritis. Typically after the anterior cruciate is severed, the animals develop arthritis within several weeks. The introduction of the fibrosis-inhibiting drug combinations such as amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate, into the joint may greatly retard the arthritic process and protect not only the cartilage, but also the underlying bone, from breakdown.

The present invention addresses a significant unmet medical need: the prevention of progressive joint degeneration after traumatic injury. Introduction of a composition containing a fibrosis-inhibiting drug combination or individual component(s) thereof into a damaged joint shortly after injury (e.g., through intra-articular injection, peri-articular administration, via arthroscope, as a joint lavage during open surgical procedures) will impact the cascade of events that lead to joint destruction, such as inhibiting inflammation and preventing cartilage matrix destruction. Most ligament injuries are severe enough or painful enough that patients seek immediate medical attention (within the first 24 to 48 hours); long before irreversible changes have occurred in the joint. If at the time of initial presentation to a health care professional, an intra-articular injection of a fibrosis-inhibitor can be administered into the joint to stop or slow down the destructive activity (in the joint and the tissues surrounding the joint), the articular cartilage can be protected from breakdown. Early introduction of the agents of the present invention intervention will slow, decrease or eliminate the cascade of events that lead to osteoarthritis. The invention can be administered immediately after injury, repeated during the period leading up to stabilization surgery, and/or can be administered after surgery is completed.

Thus, within one aspect of the present invention, methods are provided for treating or preventing cartilage loss, comprising the step of administering to a patient in need thereof a therapeutically effective amount of an anti-scarring drug combination (or individual component(s) thereof) or a composition comprising an anti-scarring drug combination (or individual component(s) thereof).

An effective anti-scarring therapy for cartilage loss will accomplish one or more of the following: (i) decrease the severity of symptoms (pain, swelling and tenderness of affected joints; (ii) decrease the severity of clinical signs of the disease (thickening of the joint capsule, synovial hypertrophy, joint effusion, soft tissue contractures, decreased range of motion, ankylosis and fixed joint deformity); (iii) increase the frequency and duration of disease remission/symptom-free periods; (iv) delay or prevent the onset of clinically significant arthritis in a joint that has previously been injured; and/or (v) prevent or reduce fixed impairment and disability.

According to the present invention, any anti-scarring drug combination (or individual component(s) thereof) described above could be utilized in the practice of this invention. Within certain embodiments of the invention, the composition may release an agent that inhibits one or more of the general components of the process of fibrosis (or scarring) associated with joint damage, including: (a) formation of new blood vessels (angiogenesis), (b) migration and/or proliferation of connective tissue cells (such as fibroblasts or synoviocytes), (c) deposition and remodeling of extracellular matrix (ECM) by matrix metalloproteinase activity, (d) inflammatory response by cytokines (such as IL-1, TNFα, FGF, VEGF). By inhibiting one or more of the components of fibrosis (or scarring), joint damage and osteoarthritis development may be reduced or prevented in a previously injured joint.

In one aspect, the composition includes an anti-scarring drug combination (or individual component(s) thereof) and a polymeric carrier suitable for application to treat an injured joint. Numerous polymeric and non-polymeric delivery systems and compositions containing an anti-scarring drug combination (or individual component(s) thereof) for use in the prevention of cartilage loss have been described above. An anti-scarring drug combination or individual component(s) thereof may be administered systemically (orally, intravenously, or by intramuscular or subcutaneous injection) in the minimum dose to achieve the above mentioned results. For patients with only a small number of joints affected, or with disease more prominent in a limited number of joints, the anti-scarring drug combination or individual component(s) thereof can be applied onto tissue within a joint or directly injected into the affected joint (intraarticular injection).

The anti-scarring drug combination or individual component(s) thereof can be administered in any manner described herein. However, preferred methods of administration include intravenous, oral, or subcutaneous, intramuscular or intra-articular injection. The anti-scarring drug combination or individual component(s) thereof can be directly injected into the affected joint (intra-articular injection) via percutaneous needle insertion into the joint capsule, or as part of an arthroscopic procedure performed on the joint. In a preferred embodiment, the intra-articular formulation containing a fibrosis-inhibiting drug combination or individual component(s) thereof is administered to a joint following an injury with a high probability of inducing subsequent arthritis (e.g., cruciate ligament tears in the knee, meniscal tears in the knee). The fibrosis-inhibiting drug combination or individual component(s) thereof is administered for a period sufficient (either through sustained release preparations and/or repeated injections) to protect the cartilage from breakdown as a result of the injury (or the surgical procedure used to treat it). The anti-scarring drug combination or individual component(s) thereof can be administered as a chronic low dose therapy to prevent disease progression, prolong disease remission, or decrease symptoms in active disease. Alternatively, the therapeutic agent can be administered in higher doses as a “pulse” therapy to induce remission in acutely active disease (such as in the period immediately following a joint injury). The minimum dose capable of achieving these endpoints can be used and can vary according to patient, severity of disease, formulation of the administered agent, clearance from the joint, potency and/or tolerability of the agent, and route of administration.

A variety of commercially available HA compositions for intra-articular injection may be combined with one or more drug combinations or individual components thereof according to the present invention including: SYNVISC (Biomatrix, Inc., Ridgefield, N.J.)—an elastoviscous fluid containing hylan (a derivative of sodium hyaluronate (hyaluronan)) polymers derived from rooster combs, HYALGAN (Sanofi-Synthelabo Inc. New York, N.Y.), and ORTHOVISC (Ortho Biotech Products, Bridgewater, N.J.)—a highly purified, high molecular weight, high viscosity injectable form of HA intended to relieve pain and to improve joint mobility and range of motion in patients suffering from osteoarthritis (OA) of the knee. ORTHOVISC is injected into the knee to restore the elasticity and viscosity of the synovial fluid. HYVISC is a high molecular weight, injectable HA product developed by Anika Therapeutics (Woburn, Mass.) currently being used to treat osteoarthritis and lameness in racehorses. Other HA-based viscosupplementation products for intra-articular injection include SUPARTZ from Seikagaku Corp. (Japan), SUPLASYN from Bioniche Life Sciences, Inc. (Canada), ARTHREASE from DePuy Orthopaedics, Inc. (Warsaw, Ind.), and DUROLANE from Q-Med AB (Sweden). By adding a fibrosis-inhibiting agent to the HA, the intra-articular injection has the added benefit of helping to prevent cartilage breakdown (i.e., it is “chondroprotective”).

In one aspect, the compositions of the present invention may be used for the management of osteoarthritis in animals (e.g., horses). It should be noted that some HA products (notably HYVISC by Boehringer Ingelheim Vetmedica, St. Joseph, Mo.) are used in veterinary applications (typically in horses to treat osteoarthritis and lameness).

Fibrosis-inhibiting formulations that can be used for the treatment or prevention of cartilage loss may be in the form of solutions, topical formulations (e.g., solution, cream, ointment, gel) emulsions, micellar solutions, gels (crosslinked and non-crosslinked), suspensions and/or pastes. One form for the formulation is as an injectable composition for intra-articular or arthroscopic delivery. For compositions that further contain a polymer to increase the viscosity of the formulation, hyaluronic acid (crosslinked, derivatized and/or non-crosslinked) is an exemplary material. These formulations can further comprise additional polymers (e.g., collagen, poly(ethylene glycol) or dextran) as well as biocompatible solvents (e.g., ethanol, DMSO, or NMP). In one embodiment, the fibrosis-inhibiting drug combination or individual component(s) thereof can be incorporated directly into the formulation. In another embodiment, the fibrosis-inhibiting drug combination or individual component(s) thereof can be incorporated into a secondary carrier (e.g., micelles, liposomes, emulsions, microspheres, nanospheres etc, as described above). The microsphere and nanospheres may be comprised of degradable polymers. Degradable polymers that can be used include poly(hydroxyl esters) (e.g., PLGA, PLA, PCL, and the like), as well as polyanhydrides, polyorthoesters and polysaccharides (e.g., chitosan and alginates).

In one embodiment, the fibrosis-inhibiting composition further comprises a polymer where the polymer is a degradable polymer. The degradable polymers may include polyesters where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator. In another embodiment, the fibrosis-inhibiting composition may further comprise a solvent, a liquid oligomer or liquid polymer such that the final composition may be passed through a 18G needle. The reagents that may be used include ethanol, NMP, PEG 200, PEG 300 and low molecular weight liquid polymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator.

In another embodiment, the fibrosis-inhibiting drug combination or individual component(s) thereof may be in the form of a solution or suspension in an organic solvent, a liquid oligomer or a liquid polymer. In this embodiment, reagents such as ethanol, NMP, PEG 200, PEG 300 and low molecular weight liquid polymers of the form X-Y, Y-X-Y, R-(Y-X)ₙ, R-(X-Y)ₙ and X-Y-X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, may be used.

Examples of fibrosis-inhibiting drug combinations for use in the treatment of, or prevention of, cartilage loss following traumatic injury include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for the treatment of cartilage loss will depend on a variety of factors, including the type of formulation and treatment site. However, certain principles can be applied in the application of this art. Drug dose can be calculated as a function of dose per unit area (of the treatment site), total drug dose administered can be measured and appropriate surface concentrations of active drug can be determined. For local application (such as intra-articular or endoscopic administration), drugs are to be used at concentrations that range from several times more than to 50%, 20%, 10%, 5%, or even less than 1% of the concentration typically used in a single systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days. It should be understood in certain embodiments that within the drug combination, one drug may be released at a different rate and/or for a different amount of time than the other drug(s).

The exemplary drug combinations or individual components thereof should be administered under the following dosing guidelines. The total amount (dose) of anti-scarring agent(s) in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations that can be used in conjunction with compositions for the treatment of cartilage loss in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500,and 1:1000.

In certain embodiments, any anti-infective agent described above may be used in conjunction with anti-fibrosis drug combinations or compositions for the treatment or prevention of cartilage loss. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

(d) Hypertrophic Scars/Keloids

In another aspect of the invention, anti-fibrosis drug combinations, compositions comprising the drug combinations and methods are provided for treating hypertrophic scars and keloids.

Hypertrophic scars and keloids are an overgrowth of dense fibrous tissue that is the result of an excessive fibroproliferative wound healing process. Hypertrophic scars and keloids usually develop after healing of a skin injury. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months.

If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including bums, open wounds, and infected wounds. With hypertrophic scars, some degree of maturation occurs and gradual improvement occurs. In the case of keloids however, an actual tumor is produced which can become quite large. Spontaneous improvement in such cases rarely occurs.

Keloids and hypertrophic scars located at most sites are primarily of cosmetic concern; however, some keloids or hypertrophic scars can cause contractures, which may result in a loss of function if overlying a joint, or they can cause significant disfigurement if located on the face. Both keloids and hypertrophic scars can be painful or pruritic.

Within one embodiment of the present invention the compositions are directly injected into a hypertrophic scar or keloid, in order to prevent the progression of these lesions. The frequency of injections will depend upon the release kinetics of the polymer used, and the clinical response. This therapy is of particular value in the prophylactic treatment of conditions which are known to result in the development of hypertrophic scars and keloids (e.g., bums, the excision site of a keloid or hypertrophic scar, wounds on the chest and back of predisposed patients, etc.), and is preferably initiated prior to, or during the proliferative phase (from day 1 forward), but before hypertrophic scar or keloid development (i.e., within the first 3 months post-injury).

In one aspect, the present invention provides topical and injectable compositions that include an anti-scarring drug combination or individual component(s) thereof and a polymeric carrier suitable for application on or into hypertrophic scars or keloids. Numerous polymeric and non-polymeric delivery systems for use in treating hypertrophic scars or keloids have been described above.

Incorporation of a fibrosis-inhibiting drug combination or individual component(s) thereof into a topical formulation or an injectable formulation is one approach to treat this condition. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are described above.

In another embodiment, the fibrosis-inhibiting drug combination or individual component(s) thereof can be incorporated into a secondary carrier (e.g., micelles, liposomes, emulsions, microspheres, nanospheres etc, as described above). Microsphere and nanospheres may include degradable polymers. Degradable polymers that can be used include poly(hydroxyl esters) (e.g., PLGA, PLA, PCL, and the like) as well as polyanhydrides, polyorthoesters and polysaccharides (e.g., chitosan and alginates).

In addition, a variety of other compositions and approaches for treating hypertrophic scars and keloids may be used in combination with the compositions of the present invention. For example, treatment may include the administration of an effective amount of angiogenesis inhibitor (e.g., fumagillol, thalidomide) as a systemic or local treatment to decrease excessive scarring. See, e.g., U.S. Pat. No. 6,638,949. The treatment may be a copolymer composed of a hydrophilic polymer, such as polyethylene glycol, that is bound to a polymer that adsorbs readily to the surfaces of body tissues, such as phenylboronic acid. See, e.g., U.S. Pat. No. 6,596,267. The treatment may include a cryoprobe containing cryogen whereby it is positioned within the hypertrophic scar or keloid to freeze the tissue. See, e.g., U.S. Pat. No. 6,503,246. The treatment may be a method of locally administering an amount of botulinum toxin in or in close proximity to the skin wound, such that the healing is enhanced. See, e.g., U.S. Pat. No. 6,447,787. The treatment may be a liquid composition composed of a film-forming carrier such as a collodion which contains one or more active ingredients such as a topical steroid, silicone gel and vitamin E. See, e.g., U.S. Pat. No. 6,337,076. The treatment may be a method of administering an antifibrotic amount of fluoroquinolone to prevent or treat scar tissue formation. See, e.g., U.S. Pat. No. 6,060,474. The treatment may be a composition of an effective amount of calcium antagonist and protein synthesis inhibitor sufficient to cause matrix degradation at a scar site so as to control scar formation. See, e.g., U.S. Pat. No. 5,902,609. The treatment may be a composition of non-biodegradable microspheres with a substantial surface charge in a pharmaceutically acceptable carrier. See, e.g., U.S. Pat. No. 5,861,149. The treatment may be a composition of endothelial cell growth factor and heparin which may be administered topically or by intralesional injection. See, e.g., U.S. Pat. No. 5,500,409.

Treatments and compositions for hypertrophic scars and keloids, which may be combined with one or more fibrosis-inhibiting drug combination or individual component(s) thereof according to the present invention, include commercially available products. Representative products include, for example, PROXIDERM External Tissue Expansion product for wound healing from Progressive Surgical Products (Westbury, N.Y.), CICA-CARE Gel Sheet dressing product from Smith & Nephew Healthcare Ltd (India), and MEPIFORM Self-Adherent Silicone Dressing from Molnlycke Health Care (Eddystone, Pa.).

In one aspect, the present invention provides topical and injectable compositions that include an anti-scarring drug combination or individual component(s) thereof and a polymeric carrier suitable for application on or into hypertrophic scars or keloids or sites that are prone to forming hypertrophic scars or keloids.

Within one embodiment of the present invention, anti-fibrosis drug combinations or individual components thereof are directly injected into a hypertrophic scar or keloid, in order to prevent the progression of these lesions. The frequency of injections will depend upon the release kinetics of the polymer used (if present), and the clinical response. This therapy is of particular value in the prophylactic treatment of conditions which are known to result in the development of hypertrophic scars and keloids (e.g., burns, the excision site of a keloid or hypertrophic scar, wounds on the chest and back of predisposed patients, etc.), and is preferably initiated prior to, or during the proliferative phase (from day 1 forward), but before hypertrophic scar or keloid development (i.e., within the first 3 months post-injury).

According to the present invention, any fibrosis-inhibiting drug combination or individual component(s) thereof described above could be utilized in the practice of this embodiment. Within one embodiment of the invention, compositions for treating hypertrophic scars or keloids may release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Examples of fibrosis-inhibiting drug combinations for use in composition for treating hypertrophic scars and keloids include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for the treatment of hypertrophic scars and keloids will depend on a variety of factors, including the type of formulation and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring drug combination or individual component(s) thereof is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing drug combinations should be administered under the following dosing guidelines. The total amount (dose) of anti-scarring agent(s) in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations that can be used in conjunction with compositions for treating hypertrophic scars and keloids in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

In certain embodiments, any anti-infective agent described above may be used in conjunction with anti-fibrosis drug combinations or compositions for the treatment or prevention of hypertrophic scars and keloids. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

(e) Vascular Diseases

In one aspect, the present invention provides for the use of an anti-fibrosis drug combination or a composition comprising the drug combination for the treatment of vascular disease (e.g., stenosis, restenosis, or atherosclerosis).

Perivascular Delivery

A further aspect of the invention provides therapeutic compositions which may be delivered perivascularly (e.g., to an external portion of a blood vessel or directly into the adventitia of a blood vessel) for the treatment or prevention of a vascular disease (e.g., stenosis, restenosis, or atherosclerosis).

Perivascular drug delivery involves percutaneous administration of localized (often sustained release) therapeutic formulations using a needle or catheter directed via ultrasound, CT, fluoroscopic, MRI or endoscopic guidance to the adventitial surface of a targeted blood vessel (arteries, veins, autologous bypass grafts, synthetic bypass grafts, AV fistulas). Alternatively the procedure can be performed intra-operatively (e.g., during bypass surgery, hemodialysis access surgery) under direct vision or with additional imaging guidance. Such a procedure can also be performed in conjunction with endovascular procedures such as angioplasty, atherectomy, or stenting or in association with an operative arterial procedure such as endarterectomy, vessel or graft repair or graft insertion.

For example, within one embodiment, an anti-fibrosis drug combination or individual component(s) thereof can be injected into the vascular wall or applied to the adventitial surface of a blood vessel allowing drug concentrations to remain highest in regions where biological activity is most needed. This has the potential to reduce local “washout” of the drug that can be accentuated by continuous blood flow over the surface of an endovascular drug delivery device (such as a drug-coated stent). Administration of effective fibrosis-inhibiting drug combination or individual component(s) thereof to the external surface of the vessel can reduce obstruction of the artery, vein or graft and reduce the risk of complications associated with intravascular manipulations (such as restenosis, embolization, thrombosis, plaque rupture, and systemic drug toxicity).

For example, in a patient with narrowing of the superficial femoral artery, balloon angioplasty would be performed in the usual manner (i.e., passing a balloon angioplasty catheter down the artery over a guide wire and inflating the balloon across the lesion). Prior to, at the time of, or after angioplasty, a needle would be inserted through the skin under ultrasound, fluoroscopic, or CT guidance and a fibrosis-inhibiting drug combination or composition would be infiltrated through the needle or catheter in a circumferential manner directly around the area of narrowing in the artery. This could be performed around any artery, vein or graft, but ideal candidates for this intervention include diseases of the carotid, coronary, iliac, common femoral, superficial femoral and popliteal arteries and at the site of graft anastomosis. Logical venous sites include infiltration around veins in which indwelling catheters are inserted. Similarly at the time of endoscopic or open coronary bypass surgery, peripheral bypass surgery or hemodialysis access surgery, a fibrosis-inhibiting drug combination or composition would be infiltrated, sprayed or wrapped in a circumferential manner in the region of the anastomosis where there is an increased incidence of restenosis. This could be performed around any artery, vein or graft, but ideal candidates for this intervention include diseases of the carotid, coronary, iliac, common femoral, superficial femoral and popliteal arteries and at the site of AV graft anastomosis.

According to the present invention, any anti-scarring drug combination or individual component(s) thereof described above can be utilized in the practice of this invention. Within one embodiment, compositions for perivascular drug delivery may be adapted to release an agent that inhibits one or more of the five general components of the process of fibrosis (or scarring), including: inflammatory response and inflammation, migration and proliferation of connective tissue cells (such as fibroblasts or smooth muscle cells), formation of new blood vessels (angiogenesis), deposition of extracellular matrix (ECM), and remodeling (maturation and organization of the fibrous tissue). By inhibiting one or more of the components of fibrosis (or scarring), the overgrowth of neointimal tissue may be inhibited or reduced.

The drug dose of the fibrosis-inhibiting drug combination or individual component(s) thereof administered from the present compositions for perivascular delivery will depend on a variety of factors, including the type of formulation and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring drug combination or individual component(s) thereof is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations for use with compositions for perivascular drug delivery include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The exemplary anti-fibrosing drug combination or individual component(s) thereof should be administered under the following dosing guidelines. The total amount (dose) of anti-scarring drug combination or individual component(s) thereof in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combination that can be used in conjunction with perivascular administration in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

According to another aspect, any anti-infective agent described above may be used alone or in conjunction with an anti-fibrosing agent in the practice of the present embodiment. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁴ M of the agent is maintained on the tissue surface.

(f) Combining With Medical Devices or Implants

The fibrosis-inhibiting drug combinations (or individual components thereof) and compositions comprising the fibrosis-inhibiting drug combinations (or individual components thereof) of the present invention can also be combined with an implant or an implantable medical device, (e.g., artificial joints, retaining pins, cranial plates, and the like, of metal, plastic and/or other materials), breast implants (e.g., silicone gel envelopes, foam forms, and the like), implanted catheters and cannulas intended for long-term use (beyond about three days), artificial organs and vessels (e.g., artificial hearts, pancreases, kidneys, blood vessels, and the like), drug delivery devices (including monolithic implants, pumps and controlled release devices such as ALZET minipumps (DURECT Corporation, Cupertino, Calif.), steroid pellets for anabolic growth or contraception, and the like, sutures for dermal or internal use, periodontal membranes, ophthalmic shields, corneal lenticules, and the like.

A range of polymeric and non-polymeric materials can be used to incorporate the fibrosis-inhibiting drug combination or individual component(s) thereof onto or into a device. The anti-fibrosing drug combination or individual component(s) thereof can be incorporated into or onto the device in a variety of ways. Coating of the device with the fibrosis-inhibiting drug combination or individual component(s) thereof is one process that can be used to incorporate the fibrosis-inhibiting drug combination or individual component(s) thereof into or onto the device. The anti-fibrosing drug combination or individual component(s) thereof may be coated onto the entire device or a portion of the device using a method, such as by dipping, spraying, painting or vacuum deposition, that is appropriate for the particular type of device.

1) Dip Coating

Dip coating is one coating process that can be used. In one embodiment, the fibrosis-inhibiting drug combination or individual component(s) thereof is dissolved in a solvent for the fibrosis-inhibiting drug combination or individual component(s) thereof and is then coated onto the device.

Fibrosis-inhibiting Drug Combination or Individual Component(s) Thereof with an Inert-solvent

In one embodiment, the solvent is an inert solvent for the device such that the solvent does not dissolve the medical device to any great extent and is not absorbed by the device to any great extent. The device can be immersed, either partially or completely, in the resulting solution for a specific period of time. The rate of immersion into the solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting drug combination or individual component(s) thereof being coated on the surface of the device.

Fibrosis-inhibiting Drug Combination or Individual Component(s) Thereof with a Swelling Solvent

In one embodiment, the solvent is one that will not dissolve the device but will be absorbed by the device. These solvents can thus swell the device to some extent. The device can be immersed, either partially or completely, in the resulting solution for a specific period of time (seconds to days). The rate of immersion into the resulting agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting drug combination or individual component(s) thereof being adsorbed into the medical device. The fibrosis-inhibiting drug combination or individual component(s) thereof may also be present on the surface of the device. The amount of surface associated fibrosis-inhibiting drug combination or individual component(s) thereof may be reduced by dipping the coated device into a solvent for the fibrosis-inhibiting drug combination or individual component(s) thereof or by spraying the coated device with a solvent for the fibrosis-inhibiting drug combination or individual component(s) thereof.

Fibrosis-inhibiting Drug Combination or Individual Component(s) Thereof with a Solvent

In one embodiment, the solvent is one that will be absorbed by the device and that will dissolve the device. The device can be immersed, either partially or completely, in the resulting solution for a specific period of time (seconds to hours). The rate of immersion into the solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting drug combination or individual component(s) thereof being adsorbed into the medical device as well as being surface associated. In the preferred embodiment, the exposure time of the device to the solvent can be such that there are no significant permanent dimensional changes to the device. The fibrosis-inhibiting drug combination or individual component(s) thereof may also be present on the surface of the device. The amount of surface associated fibrosis-inhibiting drug combination or individual component(s) thereof may be reduced by dipping the coated device into a solvent for the fibrosis-inhibiting drug combination or individual component(s) thereof or by spraying the coated device with a solvent for the fibrosis-inhibiting drug combination or individual component(s) thereof.

In the above description the device can be a device that has not been modified as well as a device that has been further modified by coating with a polymer, surface treated by plasma treatment, flame treatment, corona treatment, surface oxidation or reduction, surface etching, mechanical smoothing or roughening, or grafting prior to the coating process.

In one embodiment, the fibrosis-inhibiting drug combination or individual component(s) thereof and a polymer are dissolved in a solvent, for both the polymer and the fibrosis-inhibiting drug combination or individual component(s) thereof, and are then coated onto the device.

In any one the above dip coating methods, the surface of the device can be treated with a plasma polymerization method prior to coating of the scarring agent or scarring agent containing composition, such that a thin polymeric layer is deposited onto the device surface. Examples of such methods include parylene coating of devices and the use of various monomers such hydrocyclosiloxane monomers. Parylene coating may be especially advantageous if the device, or portions of the device, is composed of materials (e.g., stainless steel, nitinol) that do not allow incorporation of the therapeutic agent(s) into the surface layer using one of the above methods. A parylene primer layer may be deposited onto the device using a parylene coater (e.g., PDS 2010 LABCOTER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and PARYLENE D, a dichlorinated derivative of PARYLENE N).

Fibrosis-inhibiting Drug Combination (or Individual Component(s) Thereof)Polymer with an Inert-solvent

In one embodiment, the solvent is an inert solvent for the device such that the solvent does not dissolve the medical device to any great extent and is not absorbed by the device to any great extent. The device can be immersed, either partially or completely, in a fibrosis-inhibiting drug combination (or individual component(s) thereof)/polymer/solvent solution for a specific period of time. The rate of immersion into the fibrosis-inhibiting drug combination (or individual component(s) thereof)/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting drug combination (or individual component(s) thereof)/polymer being coated on the surface of the device.

Fibrosis-inhibiting Drug Combination (or Individual Component(s) Thereof)/Polymer with a Swelling Solvent

In one embodiment, the solvent is one that will not dissolve the device but will be absorbed by the device. These solvents can thus swell the device to some extent. The device can be immersed, either partially or completely, in a fibrosis-inhibiting drug combination (or individual component(s) thereof)/polymer/solvent solution for a specific period of time (seconds to days). The rate of immersion into the fibrosis-inhibiting drug combination (or individual component(s) thereof)/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting drug combination (or individual component(s) thereof)/polymer being coated onto the surface of the device as well as the potential for the fibrosis-inhibiting drug combination (or individual component(s) thereof) being adsorbed into the medical device. The fibrosis-inhibiting drug combination (or individual component(s) thereof) may also be present on the surface of the device. The amount of surface associated fibrosis-inhibiting drug combination (or individual component(s) thereof) may be reduced by dipping the coated device into a solvent for the fibrosis-inhibiting drug combination (or individual component(s) thereof) or by spraying the coated device with a solvent for the fibrosis-inhibiting drug combination (or individual component(s) thereof).

Fibrosis-inhibiting Drug Combination (or Individual Component(s) Thereof)/Polymer with a Solvent

In one embodiment, the solvent is one that will be absorbed by the device and that will dissolve the device. The device can be immersed, either partially or completely, in a fibrosis-inhibiting drug combination (or individual component(s) thereof)/polymer/solvent solution for a specific period of time (seconds to hours). The rate of immersion into the fibrosis-inhibiting drug combination (or individual component(s) thereof)/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. In the preferred embodiment, the exposure time of the device to the solvent can be such that there are not significant permanent dimensional changes to the device (other than those associated with the coating itself). The fibrosis-inhibiting drug combination (or individual component(s) thereof) may also be present on the surface of the device. The amount of surface associated fibrosis-inhibiting drug combination (or individual component(s) thereof) may be reduced by dipping the coated device into a solvent for the fibrosis-inhibiting drug combination (or individual component(s) thereof) or by spraying the coated device with a solvent for the fibrosis-inhibiting drug combination (or individual component(s) thereof).

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting drug combination (or individual component(s) thereof) in a polymer solution can be prepared. The suspension can be prepared by choosing a solvent that can dissolve the polymer but not the fibrosis-inhibiting drug combination (or individual component(s) thereof) or a solvent that can dissolve the polymer and in which the fibrosis-inhibiting agent is above its solubility limit. In similar processes described above, a device can be dipped into the suspension of the fibrosis-inhibiting drug combination (or individual component(s) thereof) and polymer solution such that the device is coated with a polymer that has a fibrosis-inhibiting drug combination (or individual component(s) thereof) suspended within it.

2) Spray Coating

Spray coating is another coating process that can be used. In the spray coating process, a solution or suspension of the fibrosis-inhibiting drug combination (or individual component(s) thereof), with or without a polymeric or non-polymeric carrier, is nebulized and directed to the device to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosis-inhibiting drug combination (or individual component(s) thereof) is dissolved in a solvent for the anti-fibrosis drug combination (or individual component(s) thereof) and is then sprayed onto the device.

Fibrosis-inhibiting Drug Combination (or Individual Component(s) Thereof) with an Inert-solvent

In one embodiment, the solvent is an inert solvent for the device such that the solvent does not dissolve the medical device to any great extent and is not absorbed by the device to any great extent. The device can be held in place or the device can be mounted onto a mandrel or rod that has the ability to move in an X, Y or Z plane or a combination of these planes. Using one of the above described spray devices, the device can be spray coated such that the device is either partially or completely coated with the fibrosis-inhibiting drug combination (or individual component(s) thereof)/solvent solution. The rate of spraying of the fibrosis-inhibiting drug combination (or individual component(s) thereof)/solvent solution can be altered (e.g., 0.001 mL per sec to 10 mL per sec) to ensure that a good coating of the fibrosis-inhibiting drug combination (or individual component(s) thereof)is obtained. The coated device can be air-dried. The spray coating process can be repeated one or more times depending on the specific application. The device can be dried under vacuum to reduce residual solvent levels. This process will result in the fibrosis-inhibiting drug combination (or individual component(s) thereof) being coated on the surface of the device.

Fibrosis-inhibiting Drug Combination (or Individual Component(s) Thereof) with a Swelling Solvent

In one embodiment, the solvent is one that will not dissolve the device but will be absorbed by the device. These solvents can thus swell the device to some extent. The device can be spray coated, either partially or completely, in the fibrosis-inhibiting drug combination (or individual component(s) thereof)/solvent solution. The rate of spraying of the fibrosis-inhibiting drug combination (or individual component(s) thereof)/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. This process will result in the fibrosis-inhibiting drug combination (or individual component(s) thereof) being adsorbed into the medical device. The fibrosis-inhibiting drug combination (or individual component(s) thereof) may also be present on the surface of the device. The amount of surface associated fibrosis-inhibiting drug combination (or individual component(s) thereof) may be reduced by dipping the coated device into a solvent for the fibrosis-inhibiting drug combination (or individual component(s) thereof) or by spraying the coated device with a solvent for the fibrosis-inhibiting drug combination (or individual component(s) thereof).

Fibrosis-inhibiting Drug Combination (or Individual Component(s) Thereof) with a Solvent

In one embodiment, the solvent is one that will be absorbed by the device and that will dissolve the device. The device can be spray coated, either partially or completely, in the fibrosis-inhibiting drug combination (or individual component(s) thereof)/solvent solution. The rate of spraying of the fibrosis-inhibiting drug combination (or individual component(s) thereof)/solvent solution can be altered (e.g., 0.001 mL per sec to 10 mL per sec) to ensure that a good coating of the fibrosis-inhibiting drug combination (or individual component(s) thereof) is obtained. The coated device can be air-dried. The spray coating process can be repeated one or more times depending on the specific application. The device can be dried under vacuum to reduce residual solvent levels. This process will result in the fibrosis-inhibiting drug combination (or individual component(s) thereof) being adsorbed into the medical device as well as being surface associated. In the preferred embodiment, the exposure time of the device to the solvent can be such that there are not significant permanent dimensional changes to the device. The fibrosis-inhibiting drug combination (or individual component(s) thereof) may also be present on the surface of the device. The amount of surface associated fibrosis-inhibiting drug combination (or individual component(s) thereof) may be reduced by dipping the coated device into a solvent for the fibrosis-inhibiting drug combination (or individual component(s) thereof) or by spraying the coated device with a solvent for the fibrosis-inhibiting drug combination (or individual component(s) thereof).

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosis-inhibiting drug combination (or individual component(s) thereof) and a polymer are dissolved in a solvent, for both the polymer and the anti-fibrosing drug combination (or individual component(s) thereof), and are then spray coated onto the device.

Fibrosis-inhibiting Drug Combination (or Individual Component(s) Thereof)/Polymer with an Inert-solvent

In one embodiment, the solvent is an inert solvent for the device such that the solvent does not dissolve the medical device to any great extent and is not absorbed by the device to any great extent. The device can be spray coated, either partially or completely, in a fibrosis-inhibiting drug combination (or individual component(s) thereof)/polymer/solvent solution for a specific period of time. The rate of spraying of the fibrosis-inhibiting drug combination (or individual component(s) thereof)/polymer/solvent solution can be altered (e.g., 0.001 mL per sec to 10 mL per sec) to ensure that a good coating of the fibrosis-inhibiting drug combination (or individual component(s) thereof) is obtained. The coated device can be air-dried. The spray coating process can be repeated one or more times depending on the specific application. The device can be dried under vacuum to reduce residual solvent levels. This process will result in the fibrosis-inhibiting drug combination (or individual component(s) thereof)/polymer being coated on the surface of the device.

Fibrosis-inhibiting Drug Combination (or Individual Component(s) Thereof)/Polymer with a Swelling Solvent

In one embodiment, the solvent is one that will not dissolve the device but will be absorbed by the device. These solvents can thus swell the device to some extent. The device can be spray coated, either partially or completely, in a fibrosis-inhibiting drug combination (or individual component(s) thereof)/polymer/solvent solution. The rate of spraying of the fibrosis-inhibiting drug combination (or individual component(s) thereof)/polymer/solvent solution can be altered (e.g., 0.001 mL per sec to 10 mL per sec) to ensure that a good coating of the fibrosis-inhibiting drug combination (or individual component(s) thereof) is obtained. The coated device can be air-dried. The spray coating process can be repeated one or more times depending on the specific application. The device can be dried under vacuum to reduce residual solvent levels. This process will result in the fibrosis-inhibiting drug combination (or individual component(s) thereof)/polymer being coated onto the surface of the device as well as the potential for the fibrosis-inhibiting drug combination (or individual component(s) thereof) being adsorbed into the medical device. The fibrosis-inhibiting drug combination (or individual component(s) thereof) may also be present on the surface of the device. The amount of surface associated fibrosis-inhibiting drug combination (or individual component(s) thereof) may be reduced by dipping the coated device into a solvent for the fibrosis-inhibiting drug combination (or individual component(s) thereof) or by spraying the coated device with a solvent for the fibrosis-inhibiting drug combination (or individual component(s) thereof).

Fibrosis-inhibiting Drug Combination (or Individual Component(s) Thereof)/Polymer with a Solvent

In one embodiment, the solvent is one that will be absorbed by the device and that will dissolve the device. The device can be spray coated, either partially or completely, in a fibrosis-inhibiting drug combination (or individual component(s) thereof)/polymer/solvent solution. The rate of spraying of the fibrosis-inhibiting drug combination (or individual component(s) thereof)/polymer/solvent solution can be altered (e.g., 0.001 mL per sec to 10 mL per sec) to ensure that a good coating of the fibrosis-inhibiting drug combination (or individual component(s) thereof) is obtained. The coated device can be air-dried. The spray coating process can be repeated one or more times depending on the specific application. The device can be dried under vacuum to reduce residual solvent levels. In the preferred embodiment, the exposure time of the device to the solvent can be such that there are not significant permanent dimensional changes to the device (other than those associated with the coating itself). The fibrosis-inhibiting drug combination (or individual component(s) thereof) may also be present on the surface of the device. The amount of surface associated fibrosis-inhibiting drug combination (or individual component(s) thereof) may be reduced by dipping the coated device into a solvent for the fibrosis-inhibiting drug combination (or individual component(s) thereof) or by spraying the coated device with a solvent for the fibrosis-inhibiting agent.

Sequential Coating Process

In other embodiments, one of the drugs of the combination can be applied as described in the dip and/or spray coating methods above and then this can be followed by a second coating process, using one of the methods described above, in which the second drug of the combination is coated onto the device.

Top Coat Process

In other embodiments, once any of the dip coating or spray coating processes described above have been completed, the drug-loaded device can be coated with a top coat of a polymer solution. This top coat can provide a means to modulate the release profiles of the drugs. The top coat can comprise the same polymer as the drug-containing coating polymer, or it can comprise a polymer of a different molecular weight or a different composition than the drug-containing coating.

In other embodiments, the top coat layer can further comprise a biologically active agent. Examples of these agents can include anti-thrombotic agents, anti-platelet agents, anti-inflammatory agents or anti-bacterial agents.

In another embodiment, the top coat can alter the surface properties of the device. For example, the top coat can provide lubricity to the surface, and/or the top coat can either enhance or decrease the surface smoothness and/or porosity.

Drug Combination Ratios

In other embodiments, the ratio of each drug in the drug combination composition that is used to drug load the device can be altered. For example, if one has a drug combination comprising drug A and drug B, then the ratio of A:B can be altered when preparing the reagents for the processes (as described above) for drug loading the devices. For illustrative purposes, one could have a ratio of A:B of 10:90, 20:80, 30:70, 40:60, 50:50, 60:40, 70:30, 80:20 and 90:10 as well an other intermediate ratios not specifically listed.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting drug combination (or individual component(s) thereof) in a polymer solution can be prepared. The suspension can be prepared by choosing a solvent that can dissolve the polymer but not the fibrosis-inhibiting drug combination (or individual component(s) thereof) or a solvent that can dissolve the polymer and in which the fibrosis-inhibiting agent is above its solubility limit. In similar processes described above, the suspension of the fibrosis-inhibiting drug combination (or individual component(s) thereof) and polymer solution can be sprayed onto the device such that the device is coated with a polymer that has a fibrosis-inhibiting drug combination (or individual component(s) thereof) suspended within it.

In a general method for coating a surface of a synthetic implant, the multifunctional compounds are exposed to the modified environment, and a thin layer of the composition is then applied to a surface of the implant before substantial inter-reaction has occurred. In one embodiment, in order to minimize cellular and fibrous reaction to the coated implant, the compounds are selected so as to result in a matrix that has a net neutral charge. Application of the compounds to the implant surface may be by extrusion, brushing, spraying, or by any other convenient means. Following application of the compounds to the implant surface, inter-reaction is allowed to continue until complete and the three-dimensional matrix is formed.

Although this method can be used to coat the surface of any type of synthetic implant, it is particularly useful for implants where reduced thrombogenicity is an important consideration, such as artificial blood vessels and heart valves, vascular grafts, vascular stents, anastomotic connector devices, and stent/graft combinations. The method may also be used to coat implantable surgical membranes (e.g., monofilament polypropylene) or meshes (e.g., for use in hernia repair). Breast implants may also be coated using the above method in order to minimize capsular contracture.

The fibrosis-inhibiting drug combination (or individual component(s) thereof) and compositions can also be coated on a suitable fibrous material, which can then be wrapped around a bone to provide structural integrity to the bone. The term “suitable fibrous material” as used herein, refers to a fibrous material which is substantially insoluble in water, non-immunogenic, biocompatible, and immiscible with the crosslinkable compositions of the invention. The fibrous material may comprise any of a variety of materials having these characteristics and may be combined with crosslinkable compositions herein in order to form and/or provide structural integrity to various implants or devices used in connection with medical and pharmaceutical uses.

The fibrosis-inhibiting drug combination (or individual component(s) thereof) and compositions of the present invention may also be used to coat lenticules, which are made from either naturally occurring or synthetic polymers.

In yet another example, the device can be coated with a polyurethane and then allowed to partially dry such that the surface is still tacky. A particulate form of the fibrosis-inhibiting drug combination (or individual component(s) thereof) or fibrosis-inhibiting drug combination (or individual component(s) thereof)/secondary carrier can then be applied to all or a portion of the tacky coating after which the device is dried.

In yet another example, the device can be coated with one of the coatings described above. A thermal treatment process can then be used to soften the coating, afterwhich the fibrosis-inhibiting drug combination (or individual component(s) thereof) or the fibrosis-inhibiting drug combination (or individual component(s) thereof)/secondary carrier is applied to the entire device or to a portion of the device (e.g., outer surface).

In one embodiment, all or a portion of the device is coated with a primer (bonding) layer and a drug release layer, as described in U.S. Patent application entitled, “Stent with Medicated Multi-Layer Hybrid Polymer Coating,” filed Sep. 16, 2003 (U.S. Ser. No. 10/662,877).

In order to develop a hybrid polymer delivery system for targeted therapy, it is desirable to be able to control and manipulate the properties of the system both in terms of physical and drug release characteristics. The active agents can be imbibed into a surface hybrid polymer layer, or incorporated directly into the hybrid polymer coating solutions. Imbibing drugs into surface polymer layers is an efficient method for evaluating polymer-drug performance in the laboratory, but for commercial production it may be preferred for the polymer and drug to be premixed in the casting mixture. Greater efficacy can be achieved by combining the two elements in the coating mixtures in order to control the ratio of active agent to polymer in the coatings. Such ratios are important parameters to the final properties of the medicated layers, i.e., they allow for better control of active agent concentration and duration of pharmacological activity.

Typical polymers used in the drug-release system can include water-insoluble cellulose esters, various polyurethane polymers including hydrophilic and hydrophobic versions, hydrophilic polymers such as polyethylene glycol (PEG), polyethylene oxide (PEO), polyvinylpyrrolidone (PVP), PVP copolymers such as vinyl acetate, hydroxyethyl methacrylate (HEMA) and copolymers such as methylmethacrylate (PMMA-HEMA), and other hydrophilic and hydrophobic acrylate polymers and copolymers containing functional groups such as carboxyl and/or hydroxyl.

Cellulose esters such as cellulose acetate, cellulose acetate propionate, cellulose acetate butyrate, cellulose acetate phthalate, and cellulose nitrate may be used. In one aspect of the invention, the anti-fibrosis drug combination (or individual component(s) thereof) is formulated with a cellulose ester. Cellulose nitrate is a preferred cellulose ester because of its compatibility with the active agents and its ability to impart non-tackiness and cohesiveness to the coatings. Cellulose nitrate has been shown to stabilize entrapped drugs in ambient and processing conditions. Various grades of cellulose nitrate are available and may be used in a coating on a device, including cellulose nitrate having a nitrogen content=11.8-12.2%. Various viscosity grades, including 3.5, 0.5 or 0.25 seconds, may be used in order to provide proper rheological properties when combined with the coating solids used in these formulations. Higher or lower viscosity grades can be used. However, the higher viscosity grades can be more difficult to use because of their higher viscosities. Thus, the lower viscosity grades, such as 3.5, 0.5 or 0.25 seconds, are generally preferred. Physical properties such as tensile strength, elongation, flexibility, and softening point are related to viscosity (molecular weight) and can decrease with the lower molecular weight species, especially below the 0.25 second grades.

The cellulose derivatives comprise hydroglucose structures. Cellulose nitrate is a hydrophobic, water-insoluble polymer, and has high water resistance properties. This structure leads to high compatibility with many active agents, accounting for the high degree of stabilization provided to drugs entrapped in cellulose nitrate. The structure of nitrocellulose is given below:

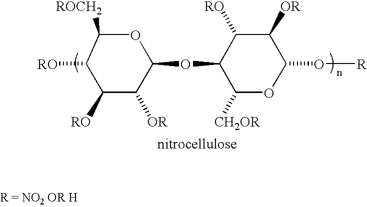

Cellulose nitrate is a hard, relatively inflexible polymer, and has limited adhesion to many polymers that are typically used to make medical devices. Also, control of drug elution dynamics is limited if only one polymer is used in the binding matrix. Accordingly, in one embodiment of the invention, the therapeutic agent is formulated with two or more polymers before being associated with the device. In one aspect, the agent is formulated with both polyurethane ((e.g., CHRONOFLEX AR, CHRONOFLEX AL, and BIONATE, PELLETHANE) and cellulose nitrate to provide a hybrid polymer drug loaded matrix. Polyurethanes provide the hybrid polymer matrix with greater flexibility and adhesion to the device, particularly when the device has been pre-coated with a primer. Polyurethanes can also be used to slow or hasten the drug elution from coatings. Aliphatic, aromatic, polytetramethylene ether glycol, and polycarbonate are among the types of polyurethanes, which can be used in the coatings. In one aspect, an anti-scarring drug combination (or individual component(s) thereof) may be incorporated into a carrier that includes a polyurethane and a cellulose derivative. A heparin complex, such as benzalkonium heparinate or tridodecylammonium heparinate), may optionally be included in the formulation.

From the structure below, it is possible to see how more or less hydrophilic polyurethane polymers may be created based on the number of hydrophilic groups contained in the polymer structures. In one aspect of the invention, the device is associated with a formulation that includes therapeutic agent, cellulose ester, and a polyurethane that is water-insoluble, flexible, and compatible with the cellulose ester.

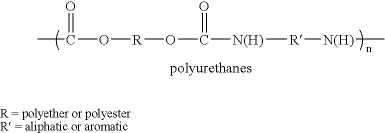

Polyvinylpyrrolidone (PVP) is a polyamide that possesses unusual complexing and colloidal properties and is essentially physiologically inert. PVP and other hydrophilic polymers are typically biocompatible. PVP may be incorporated into drug loaded hybrid polymer compositions in order to increase drug release rates. In one embodiment, the concentration of PVP that is used in drug loaded hybrid polymer compositions can be less than 20%. This concentration can not make the layers bioerodable or lubricious. In general, PVP concentrations from <1% to greater than 80% are deemed workable. In one aspect of the invention, the therapeutic agent that is associated with an device is formulated with a PVP polymer.

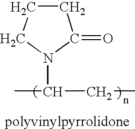

Acrylate polymers and copolymers including polymethylmethacrylate (PMMA) and polymethylmethacrylate hydroxyethyl methacrylate (PMMA/HEMA) are known for their biocompatibility as a result of their widespread use in contact and intraocular lens applications. This class of polymer generally provokes very little smooth muscle and endothelial cell growth, and very low inflammatory response (Bar). These polymers/copolymers are compatible with drugs and the other polymers and layers of the instant invention. Thus, in one aspect, the device is associated with a composition that 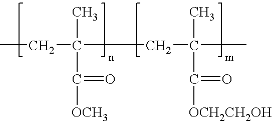
comprises an anti-scarring drug combination (or individual component(s) thereof) as described above, and an acrylate polymer or copolymer.

Methylmethacrylate Hydroxyethylmethacrylate Copolymer

Within another aspect of the invention, the coated device which inhibits or reduces an in vivo fibrotic reaction is further coated with a compound or compositions which delay the release of and/or activity of the fibrosis-inhibiting drug combination (or individual component(s) thereof). Representative examples of such agents include biologically inert materials such as gelatin, PLGA/MePEG film, PLA, polyurethanes, silicone rubbers, surfactants, lipids, or polyethylene glycol, as well as biologically active materials such as heparin (e.g., to induce coagulation).

For example, in one embodiment of the invention, the active agent on the device is top-coated with a physical barrier. Such barriers can include non-degradable materials or biodegradable materials such as gelatin, PLGA/MePEG film, PLA, or polyethylene glycol among others. In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the bloodstream, the MePEG can dissolve out of the PLGA, leaving channels through the PLGA layer to an underlying layer containing the fibrosis-inhibiting drug combination (or individual component(s) thereof), which then can then diffuse into the vessel wall and initiate its biological activity.

In another embodiment of the invention, a particulate form of the active agent may be coated onto any of the devices described below) using a polymer (e.g., PLG, PLA, aor a polyurethane). A second polymer, that dissolves slowly or degrades (e.g., MePEG-PLGA or PLG) and that does not contain the active agent, may be coated over the first layer. Once the top layer dissolves or degrades, it exposes the under coating which allows the active agent to be exposed to the treatment site or to be released from the coating.

Within another aspect of the invention, the outer layer of the coating of a coated device, which inhibits an in vivo fibrotic response, is further treated to crosslink the outer layer of the coating. This can be accomplished by subjecting the coated device to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the device surface with an inert molecule that prevents access to the active site through steric hindrance, or by coating the surface with an inactive form of the fibrosis-inhibiting drug combination (or individual component(s) thereof), which is later activated. For example, the device can be coated with an enzyme, which causes either release of the fibrosis-inhibiting drug combination (or individual component(s) thereof) or activates the fibrosis-inhibiting drug combination (or individual component(s) thereof).

In another embodiment, the device is coated with a fibrosis-inhibiting drug combination (or individual component(s) thereof) and then further coated with a composition that comprises an anticoagulant such as heparin. As the anticoagulant dissolves away, the anticoagulant activity slows or stops, and the newly exposed fibrosis-inhibiting drug combination (or individual component(s) thereof) is available to inhibit or reduce fibrosis from occurring in the adjacent tissue.

The device can be coated with an inactive form of the fibrosis-inhibiting drug combination (or individual component(s) thereof), which is then activated once the device is deployed. Such activation can be achieved by injecting another material into the treatment area after the device (as described below) is deployed or after the fibrosis-inhibiting drug combination (or individual component(s) thereof) has been administered to the treatment area (via, e.g., injections, spray, wash, drug delivery catheters or balloons). For example, the device can be coated with an inactive form of the fibrosis-inhibiting drug combination (or individual component(s) thereof). Once the device is deployed, the activating substance is injected or applied into or onto the treatment site where the inactive form of the fibrosis-inhibiting drug combination (or individual component(s) thereof) has been applied. For example, a device can be coated with a biologically active fibrosis-inhibiting drug combination (or individual component(s) thereof) and a first substance having moieties that capable of forming an ester bond with another material. The coating can be covered with a second substance such as polyethylene glycol. The first and second substances can react to form an ester bond via, e.g., a condensation reaction. Prior to the deployment of the device, an esterase is injected into the treatment site around the outside of the device, which can cleave the bond between the ester and the fibrosis-inhibiting drug combination (or individual component(s) thereof), allowing the drug combination (or individual component(s) thereof) to initiate fibrosis-inhibition.

In another aspect, a medical device may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

As described above, the anti-fibrosing drug combination (or individual component(s) thereof) can be associated with a medical device using the polymeric carriers or coatings described above. In addition to the compositions and methods described above, there are various other compositions and methods that are known in the art. Representative examples of these compositions and methods for applying (e.g., coating) these compositions to devices are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

Representative examples of medical devices which may be coated using the compositions of the invention and are described in more detail below include vascular stents, gastrointestinal stents, tracheal/bronchial stents, genital-urinary stents, ENT stents, intra-articular implants, intraocular lenses, implants for hypertrophic scars and keloids, vascular grafts, anastomotic connector devices, surgical adhesion barriers, glaucoma drainage devices, film or mesh, prosthetic heart valves, tympanostomy tubes, penile implants, endotracheal and tracheostomy tubes, peritoneal dialysis catheters, intracranial pressure monitors, vena cava filters, central venous cathethers (CVC's), ventricular assist devices (e.g., LVAD's), spinal prostheses, urinary (Foley) catheters, prosthetic bladder sphincters, orthopedic implants, and gastrointestinal drainage tubes.

There are numerous medical devices where the occurrence of a fibrotic reaction will adversely affect the functioning of the device or the biological problem for which the device was implanted or used. Representative examples of implants or devices that can be coated with or otherwise constructed to contain and/or release the therapeutic agents provided herein include cardiovascular devices (e.g., implantable venous catheters, venous ports, tunneled venous catheters, chronic infusion lines or ports, including hepatic artery infusion catheters, pacemakers and pacemaker leads, implantable defibrillators; neurologic/neurosurgical devices (e.g., ventricular peritoneal shunts, ventricular atrial shunts, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, and suspensions or solid implants to prevent surgical adhesions); genitourinary devices (e.g., uterine implants, including intrauterine devices (IUDs) and devices to prevent endometrial hyperplasia, fallopian tubal implants, including reversible sterilization devices, fallopian tubal stents, ureteric stents, chronic indwelling catheters, bladder augmentations, or wraps or splints for vasovasostomy, central venous catheters, urinary catheters; prosthetic heart valves, vascular grafts, ophthalmologic implants (e.g., multino implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants); otolaryngology devices (e.g., ossicular implants, Eustachian tube splints or stents for glue ear or chronic otitis as an alternative to transtempanic drains); catheter cuffs and orthopedic implants (e.g., cemented orthopedic prostheses).

Other examples of implants include drainage tubes, biliary T-tubes, clips, sutures, braids, meshes (e.g., hernia meshes, tissue support meshes), barriers (for the prevention of adhesions), anastomotic devices, anastomotic connectors, ventrical assist devices (e.g., LVAD's), artificial hearts, artificial joints, conduits, irrigation fluids, packing agents, stents, staples, inferior vena cava filters, pumps (e.g., for the delivery of therapeutics), hemostatic implants (e.g., sponges), tissue fillers, surgical adhesion barriers (e.g., INTERCEED, degradable polyester films (e.g., PLLA/PDLLA), CMC/PEO association complexes (e.g., OXIPLEX from Fziomed), hyaluronic acid/CMC films (e.g., SEPRAFILM from Genzyme Corporation), bone grafts, skin grafts, tissue sealants, intrauterine devices (IUD), ligatures, titanium implants (particularly for use in dental applications), chest tubes, nasogastric tubes, percutaneous feeding tubes, colostomy devices, bone wax, and Penrose drains, hair plugs, ear rings, nose rings, and other piercing-associated implants, as well as anaesthetic solutions.

The coating of fibrosis-inhibiting drug combination (or individual component(s) thereof) onto or incorporation of a fibrosis-inhibiting drug combination (or individual component(s) thereof) into medical devices provides a solution to the clinical problems that can be encountered with these devices. Alternatively, or additional, compositions that comprise anti-scarring drug combinations (or individual components thereof) can be infiltrated in to the space or onto tissue surrounding the area where medical devices are implanted either before, during or after implantation of the devices.

Described below are examples of medical devices whose functioning can be improved by the use of a fibrosis-inhibiting drug combination (or individual component(s) thereof)as well as methods for incorporating fibrosis-inhibiting the drug combination (or individual component(s) thereof) into or onto these devices and methods for using such devices.

Intravascular Devices

The present invention provides for the combination of an anti-scarring drug combination (or individual component(s) thereof) and an intravascular device. “Intravascular devices” refers to devices that are implanted at least partially within the vasculature (e.g., blood vessels). Examples of intravascular devices that can be used to deliver anti-scarring drug combination (or individual component(s) thereof) to the desired location include, e.g., catheters, balloon catheters, balloons, stents, covered stents, stent grafts, anastomotic connectors, and guidewires.

In one aspect, the present invention provides for the combination of (1) an anti-scarring drug combination (or individual component(s) thereof) or a composition comprising an anti-scarring drug combination (or individual component(s) thereof) and (2) an intravascular stent.

“Stent” refers to devices comprising a cylindrical tube (composed of a metal, textile, non-degradable or degradable polymer, and/or other suitable material (such as biological tissue) which maintains the flow of blood from one portion of a blood vessel to another. In one aspect, a stent is an endovascular scaffolding which maintains the lumen of a body passageway (e.g., an artery) and allows bloodflow. Representative examples of stents that can benefit from being coated with or having incorporated therein, a fibrosis-inhibiting drug combination (or individual component(s) thereof) include vascular stents, such as coronary stents, peripheral stents, and covered stents.

Stents that can be used in the present invention include metallic stents, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials (so called “covered stents”) or “sleeves”, and can be bare metal or drug-eluting.

Stents may be comprise a metal or metal alloy such as stainless steel, spring tempered stainless steel, stainless steel alloys, gold, platinum, super elastic alloys, cobalt-chromium alloys and other cobalt-containing alloys (including ELGILOY (Combined Metals of Chicago, Grove Village, Ill.), PHYNOX (Alloy Wire International, United Kingdom) and CONICHROME (Carpenter Technology Corporation, Wyomissing, Pa.)), titanium-containing alloys, platinum-tungsten alloys, nickel-containing alloys, nickel-titanium alloys (including nitinol), malleable metals (including tantalum); a composite material or a clad composite material and/or other functionally equivalent materials; and/or a polymeric (non-biodegradable or biodegradable) material. Representative examples of polymers that may be included in the stent construction include polyethylene, polypropylene, polyurethanes, polyesters, such as polyethylene terephthalate (e.g., DACRON or MYLAR (E. I. DuPont De Nemours and Company, Wilmington, Del.)), polyamides, polyaramids (e.g., KEVLAR from E.I. DuPont De Nemours and Company), polyfluorocarbons such as poly(tetrafluoroethylene with and without copolymerized hexafluoropropylene) (available, e.g., under the trade name TEFLON (E. I. DuPont De Nemours and Company), silk, as well as the mixtures, blends and copolymers of these polymers. Stents also may be made with engineering plastics, such as thermotropic liquid crystal polymers (LCP), such as those formed from p,p′-dihydroxy-polynuclear-aromatics or dicarboxy-polynuclear-aromatics.

Further types of stents that can be used with the described therapeutic agents are described, e.g., in PCT Publication No. WO 01/01957 and U.S. Pat. Nos. 6,165,210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in Lambert, T. (1993) J. Am. Coll. Cardiol.: 21: 483A. Moreover, the stent may be adapted to release the desired agent at only the distal ends, or along the entire body of the stent.

Balloon over stent devices, such as are described in Wilensky, R. L. (1993) J. Am. Coll. Cardiol.: 21: 185A, also are suitable for local delivery of a fibrosing agent to a treatment site.

In addition to using the more traditional stents, stents that are specifically designed for drug delivery can be used. Examples of these specialized drug delivery stents as well as traditional stents include those from Conor Medsystems (Palo Alto, Calif.) (e.g., U.S. Pat. Nos. 6,527,799; 6,293,967; 6,290,673; 6,241,762; U.S. Patent Application Publication Nos. 2003/0199970 and 2003/0167085; and PCT Publication No. WO 03/015664).

Examples of intravascular stents, which may be combined with one or more therapeutic agents according to the present invention, include commercially available products. The stent may be self-expanding or balloon expandable (e.g., STRECKER stent by Medi-Tech/Boston Scientific Corporation), or implanted by a change in temperature (e.g., nitinol stent). Self-expanding stents that can be used include the coronary WALLSTENT and the SCIMED RADIUS stent from Boston Scientific Corporation (Natick, Mass.) and the GIANTURCO stents from Cook Group, Inc. (Bloomington, Ind.). Examples of balloon expandable stents that can be used include the CROSSFLEX stent, BX-VELOCITY stent and the PALMAZ-SCHATZ crown and spiral stents from Cordis Corporation (Miami Lakes, Fla.), the V-FLEX PLUS stent by Cook Group, Inc., the NIR, EXPRESS and LIBRERTE stents from Boston Scientific Corporation, the ACS MULTILINK, MULTILINK PENTA, SPIRIT, and CHAMPION stents from Guidant Corporation, and the Coronary Stent S670 and S7 by Medtronic, Inc. (Minneapolis, Minn.).

Other examples of stents that can be combined with a fibrosing agent in accordance with the invention include those from Boston Scientific Corporation, (e.g., the drug-eluting TAXUS EXPRESS² drug-Eluting Coronary Stent System; over the wire stent stents such as the Express² Coronary Stent System and NIR Elite OTW Stent System; rapid exchange stents such as the EXPRESS² Coronary Stent System and the NIR ELITE MONORAIL Stent System; and self-expanding stents such as the MAGIC WALLSTENT Stent System and RADIUS Self Expanding Stent); Medtronic, Inc. (Minneapolis, Minn.) (e.g., DRIVER ABT578-eluting stent, DRIVER ZIPPER MX Multi-Exchange Coronary Stent System and the DRIVER Over-the-Wire Coronary Stent System; the S7 ZIPPER MX Multi-Exchange Coronary Stent System; S7, S670, S660, and BESTENT2 with Discrete Technology Over-the-Wire Coronary Stent System); Guidant Corporation (e.g., cobalt chromium stents such as the MULTI-LINK VISION Coronary Stent System; MULTI-LINK ZETA Coronary Stent System; MULTI-LINK PIXEL Coronary Stent System; MULTI-LINK ULTRA Coronary Stent System; and the MULTI-LINK FRONTIER); Johnson & Johnson/Cordis Corporation (e.g., CYPHER sirolimus-eluting Stent; PALMAZ-SCHATZ Balloon Expandable Stent; and S.M.A.R.T. Stents); Abbott Vascular (Redwood City, Calif.) (e.g., MATRIX LO Stent; TRIMAXX Stent; and DEXAMET stent); Conor Medsystems (Menlo Park, Calif.) (e.g., MEDSTENT and COSTAR stent); AMG GmbH (Germany) (e.g. PICO Elite stent); Biosensors International (Singapore) (e.g., MATRIX stent, CHAMPION Stent (formerly the S-STENT), and CHALLENGE Stent); Biotronik (Switzerland) (e.g., MAGIC AMS stent); Clearstream Technologies (Ireland) (e.g., CLEARFLEX stent); Cook Inc. (Bloomington, Ind.) (e.g., V-FLEX PLUS stent, ZILVER PTX self-expanding vascular stent coating, LOGIX PTX stent (in development); Devax (e.g., AXXESS stent) (Irvine, Calif.); DISA Vascular (Pty) Ltd (South Africa) (e.g., CHROMOFLEX Stent, S-FLEX Stent, S-FLEX Micro Stent, and TAXOCHROME DES); Intek Technology (Baar, Switzerland) (e.g., APOLLO stent); Orbus Medical Technologies (Hoevelaken, The Netherlands) (e.g., GENOUS); Sorin Biomedica (Saluggia, Italy) (e.g., JANUS and CARBOSTENT); and stents from Bard/Angiomed GmbH Medizintechnik KG (Murray Hill, N.J.), and Blue Medical Supply & Equipment (Mariettta, Ga.), Aachen Resonance GmbH (Germany); Eucatech AG (Germany), Eurocor GmbH (Bonn, Germany), Prot, Goodman, Terumo (Japan), Translumina GmbH (Germany), MIV Therapeutics (Canada), Occam International B.V. (Eindhoven, The Netherlands), Sahajanand Medical Technologies PVT LTD. (India); AVI Biopharma/Medtronic/ Interventional Technologies (Portland, Oreg.) (e.g., RESTEN NG-coated stent); and Jomed (e.g., FLEXMASTER drug-eluting stent) (Sweden).

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. A guidewire is then advanced through the lesion or proposed site of insertion, and over this is passed a delivery catheter which allows a stent in its collapsed form to be inserted. Intravascular stents may be inserted into an artery such as the femoral artery in the groin and advanced through the circulation under radiological guidance until they reach the anatomical location of the plaque in the coronary or peripheral circulation. Typically, stents are capable of being compressed, so that they can be inserted through tiny cavities via small catheters, and then expanded to a larger diameter once they are at the desired location. The delivery catheter then is removed, leaving the stent standing on its own as a scaffold. Once expanded, the stent physically forces the walls of the passageway apart and holds them open. A post insertion examination, usually an x-ray, is often utilized to confirm appropriate positioning.

Stents are typically maneuvered into place under, radiologic or direct visual control, taking particular care to place the stent precisely within the vessel being treated. In certain aspects, the stent can further include a radio-opaque, echogenic material, or MRI responsive material (e.g., MRI contrast agent) to aid in visualization of the device under ultrasound, fluoroscopy and/or magnetic resonance imaging. The radio-opaque or MRI visible material may be in the form of one or more markers (e.g., bands of material that are disposed on either end of the stent) that may be used to orient and guide the device during the implantation procedure.

In another aspect, the present invention provides for the combination of (1) an anti-scarring drug combination (or individual component(s) thereof) or a composition comprising an anti-scarring drug combination (or individual component(s) thereof) and (2) an intravascular catheter.

“Intravascular Catheter” refers to any intravascular catheter containing one or more lumens suitable for the delivery of aqueous, microparticulate, fluid, or gel formulations into the bloodstream or into the vascular wall. These formulations may contain a biologically active agent (e.g., an anti-scarring drug combination (or individual component(s) thereof)). Numerous intravascular catheters have been described for direct, site-specific drug delivery (e.g., microinjector catheters, catheters placed within or immediately adjacent to the target tissue), regional drug delivery (i.e., catheters placed in an artery that supplies the target organ or tissue), or systemic drug delivery (i.e., intra-arterial and intravenous catheters placed in the peripheral circulation). For example, catheters and balloon catheters can deliver anti-fibrosing drug combinations (or individual components thereof) from an end orifice, through one or more side ports, through a microporous outer structure, or through direct injection into the desired tissue or vascular location.

A variety of catheters are available for regional or localized arterial drug-delivery. Intravascular balloon and non-balloon catheters for delivering drugs are described, for example, in U.S. Pat. Nos. 5,180,366; 5,171,217; 5,049,132; 5,021,044; 6,592,568; 5,304,121; 5,295,962; 5,286,254; 5,254,089; 5,112,305; PCT Publication Nos WO 93/08866, WO 92/11890, and WO 92/11895; and Riessen et al. (1994) JACC 23: 1234-1244, Kandarpa K. (2000) J. Vasc. Interv. Radio. 11 (suppl.): 419-423, and Yang, X. (2003) Imaging of Vascular Gene Therapy 228(1): 36-49.

Representative examples of drug delivery catheters include balloon catheters, such as the CHANNEL and TRANSPORT balloon catheters from Boston Scientific Corporation (Natick, Mass.) and Stack Perfusion Coronary Dilitation catheters from Advanced Cardiovascular Systems, Inc. (Santa Clara, Calif.). Other examples of drug delivery catheters include infusion catheters, such as the CRESCENDO coronary infusion catheter available from Cordis Corporation (Miami Lakes, Fla.), the Cragg-McNamara Valved Infusion Catheter available from Microtherapeutics, Inc. (San Clemente, Calif.), the DISPATCH catheter from Boston Scientific Corporation, the GALILEO Centering Catheter from Guidant Corporation (Houston, Tex.), and infusion sleeve catheters, such as the INFUSASLEEVE catheter from LocalMed, Inc. (Sunnyvale, Calif.). Infusion sleeve catheters are described in, e.g., U.S. Pat. Nos. 5,318,531; 5,336,178; 5,279,565; 5,364,356; 5,772,629; 5,810,767; and 5,941,868. Catheters that mechanically or electrically enhance drug delivery include, for example, pressure driven catheters (e.g., needle injection catheters having injector ports, such as the INFILTRATOR catheter available from InterVentional Technologies, Inc. (San Diego, Calif.)) (see, e.g., U.S. Pat. No. 5,354,279) and ultrasonically assisted (phonophoresis) and iontophoresis catheters (see, e.g., Singh, J., et al. (1989) Drug Des. Deliv.: 4: 1-12 and U.S. Pat. Nos. 5,362,309; 5,318,014; 5,315,998; 5,304,120; 5,282,785; and 5,267,985).

In one aspect, the present invention provides for the combination of (1) an anti-scarring drug combination (or individual component(s) thereof)or a composition comprising an anti-scarring drug combination (or individual component(s) thereof) and (2) a drug delivery balloon.

“Drug-Delivery Balloon” refers to an intra-arterial balloon (typically based upon percutaneous angioplasty balloons) suitable for insertion into a peripheral artery (typically the femoral artery) and manipulated via a catheter to the treatment site (either in the coronary or peripheral circulation). Numerous drug delivery balloons have been developed for local delivery of therapeutic agents to the arterial wall such as “sweaty balloons,” “channel balloons,” “microinjector balloons,” “double balloons,” “spiral balloons” and other specialized drug-delivery balloons. Other examples of balloons include BHP balloons and Transurethral and Radiofrequency Needle Ablation (TUNA or RFNA)) balloons for prostate applications.

In addition, numerous drug delivery balloons have been developed for local delivery of therapeutic agents to the arterial wall. Representative examples of drug delivery balloons include porous (WOLINSKY) balloons, available from Advanced Polymers (Salem, N.H.), described in, e.g., U.S. Pat. No. 5,087,244. Microporous and macroporous balloons (i e., “sweaty balloons”) for use in infusion catheters are described in, e.g., Lambert, C. R. et al. (1992) Circ. Res. 71: 27-33. Other types of specialized drug delivery balloons include hydrogel coated balloons (e.g., ULTRATHIN GLIDES from Boston Scientific Corporation) (see, e.g., Fram, D. B. et al. (1992) Circulation: 86 Suppl. I: 1-380), “channel balloons” (see, e.g., U.S. Pat. Nos. 5,860,954; 5,843,033; and 5,254,089, and Hong, M. K., et al. (1992) Circulation: 86 Suppl. I: 1-380), “microinjector balloons” (see, e.g., U.S. Pat. Nos. 5,681,281 and 5,746,716), “double balloons,” described in, e.g., U.S. Pat. No. 6,544,221, and double-layer channeled perfusion balloons (such as the REMEDY balloon from Boston Scientific Corportion), and “spiral balloons” (see, e.g., U.S. Pat. Nos. 6,527,739 and 6,605,056). Drug delivery catheters that include helical (i.e., spiral) balloons are described in, e.g., U.S. Pat. Nos. 6,190,356; 5,279,546; 5236424, 5,226,888; 5,181,911; 4,824,436; and 4,636,195.

The balloon catheter systems that can be used include systems in which the balloon can be inflated at the desired location the desired fibrosis-inhibiting drug combination (or individual component(s) thereof) can be delivered through holes that are located in the balloon wall. Other balloon catheters that can be used include systems that have a plurality of holes that are located between two balloons. The system can be guided into the desired location such that the inflatable balloon components are located on either side of the specific site that is to be treated. The balloons can then be inflated to isolate the treatment area. The compositions containing the anti-fibrosis drug combination (or individual component(s) thereof) are then injected into the isolated area through the plurality of holes between the two balloons. Representative examples of these types of drug delivery balloons are described in U.S. Pat. Nos. 5,087,244, 6,623,452, 5,397,307, 4,636,195 and 4,994,033.

The compositions of the invention can be delivered using a catheter that has the ability to enhance uptake or efficacy of the compositions of the invention. The stimulus for enhanced uptake can include the use of heat, the use of cooling, the use of electrical fields or the use of radiation (e.g., ultraviolet light, visible light, infrared, microwaves, ultrasound or X-rays). Further Representative examples of catheter systems that can be used are described in U.S. Pat. Nos. 5,362,309 and 6,623,444; U.S. Patent Application Publication Nos. 2002/0138036 and 2002/0068869; and PCT Publication Nos. WO 01/15771; WO 94/05361; WO 96/04955 and WO 96/22111.

In another aspect of the invention, the compositions of the inventions can be delivered into the treatment site and/or into the tissue surrounding the treatment site by using catheter systems that have one or more injectors that can penetrate the surrounding tissue. Following insertion into the appropriate vessel, the catheter can be maneuvered into the desired position such that the injectors are aligned with or adjacent to the tissue. The injector(s) are inserted into the desired location, for example by direct insertion into the tissue, by inflating the balloon or mechanical rotation of the injector, and the composition of the invention is injected into the desired location. Representative examples of catheters that can be used for this application are described in and U.S. Patent Application Publication No. 2002/0082594 and U.S. Pat. Nos. 6,443,949; 6,488,659; 6,569,144; 5,609,151; 5,385,148; 5,551,427; 5,746,716; 5,681,281; and 5,713,863.

In another aspect of the invention, the catheter may be adapted to deliver a thermoreversible polymer composition. For the site-specific delivery of these materials, a catheter delivery system has the ability to either heat the composition to above body temperature or to cool the composition to below body temperature such that the composition remains in a fluent state within the catheter delivery system. The catheter delivery system can be guided to the desired location and the composition of the invention can be delivered to the surface of the surrounding tissue or can be injected directly into the surrounding tissue. A representative example of a catheter delivery system for direct injection of a thermoreversible material is described in U.S. Pat. No. 6,488,659. Representative examples of catheter delivery systems that can deliver the thermoreversible compositions to the surface of the vessel are described in U.S. Pat. Nos. 6,443,941; 6,290,729; 5,947,977; 5,800,538; and 5,749,922.

In another aspect, the present invention provides for the combination of an anti-scarring drug combination (or individual component(s) thereof) or a composition comprising an anti-scarring drug combination (or individual component(s) thereof) and an anastomotic connector device.

“Anasomotic connector device” refers to any vascular device that mechanizes the creation of a vascular anastomosis (i.e., artery-to-artery, vein-to-artery, artery-to-vein, artery-to-synthetic graft, synthetic graft-to-artery, vein-to-synthetic graft or synthetic graft-to-vein anastomosis) without the manual suturing that is typically done in the creation of an anastomosis. The term also refers to anastomotic connector devices (described below), designed to produce a facilitated semiautomatic vascular anastomosis without the use of suture and reduce connection time substantially (often to several seconds), where there are numerous types and designs of such devices. The term also refers to devices which facilitate attachment of a vascular graft to an aperture or orifice (e.g., in the side or at the end of a vessel) in a target vessel. Anastomotic connector devices may be anchored to the outside of a blood vessel, and/or into the wall of a blood vessel (e.g., into the adventitial, intramural, or intimal layer of the tissue), and/or a portion of the device may reside within the lumen of the vessel.

Anastomotic connector devices also may be used to create new flow from one structure to another through a channel or diversionary shunt. Accordingly, such devices (also referred to herein as “bypass devices”) typically include at least one tubular structure, wherein a tubular structure defines a lumen. Anastomotic connector devices may include one tubular structure or a plurality of tubular structures through which blood can flow. At least a portion of the tubular structure resides external to a blood vessel (i.e., extravascular) to provide a diversionary passageway. A portion of the device also may reside within the lumen and/or within the tissue of the blood vessel.

Examples of anastomotic connector devices are described in co-pending application entitled, “Anastomotic Connector Devices”, filed May 23, 2003 (U.S. Ser. No. 60/473,185). Representative examples of anastomotic connector devices include, without limitation, vascular clips, vascular sutures, vascular staples, vascular clamps, suturing devices, anastomotic coupling devices (i.e., anastomotic couplers), including couplers that include tubular segments for carrying blood, anastomotic rings, and percutaneous in situ coronary artery bypass (PISCAB and PICVA) devices. Broadly, anastomotic connector devices may be classified into three categories: (1) automated and modified suturing methods and devices, (2) micromechanical devices, and (3) anastomotic coupling devices.

(1) Automated and Modified Suturing Methods and Devices

Automated sutures and modified suturing methods generally facilitate the rapid deployment of multiple sutures, usually in a single step, and eliminate the need for knot tying or the use of aortic side-biting clamps. Suturing devices include those devices that are adapted to be minimally invasive such that anastomoses are formed between vascular conduits and hollow organ structures by applying sutures or other surgical fasteners through device ports or other small openings. With these devices, sutures and other fasteners are applied in a relatively quick and automated manner within bodily areas that have limited access. By using minimally invasive means for establishing anastomoses, there is less blood loss and there is no need to temporarily stop the flow of blood distal to the operating site. For example, the suturing device may be composed of a shaft-supported vascular conduit that is adapted for anastomosis and a collar that is slideable on the shaft configured to hold a plurality of needles and sutures that passes through the vascular conduit. See, e.g., U.S. Pat. No. 6,709,441. The suturing device may be composed of a carrier portion for inserting graft, arm portions that extend to support the graft into position, and a needle assembly adapted to retain and advance coil fasteners into engagement with the vessel wall and the graft flange to complete the anastomosis. See, e.g., U.S. Pat. No. 6,709,442. The suturing device may include two oblong interlinked members that include a split bush adapted for suturing (e.g., U.S. Pat. No. 4,350,160).

One representative example of a suturing device is the HEARTFLOW device, made by Perclose-Abbott Labs, Redwood City, Calif. (see generally, U.S. Pat. Nos. 6,358,258, 6,355,050, 6,190,396, and 6,036,699, and PCT Publication No. WO 01/19257)

The nitinol U-CLIP suture clip device by Coalescent Surgical (Sunnyvale, Calif.) consists of a self-closing nitinol wire loop attached to a flexible member and a needle with a quick release mechanism. This device facilitates the construction of anastomosis by simplifying suture management and eliminating knot tying (see generally, U.S. Pat. Nos. 6,074,401 and 6,149,658, and PCT Publication Nos. WO 99/62406, WO 99/62409, WO 00/59380, WO 01/17441).

The ENCLOSE Anastomotic Assist Device (Novare Surgical Systems, Cupertino, Calif.) allows a surgeon to create a sutured anastomosis using standard suturing techniques but without the use of a partial occluding side-biting aortic clamp, avoiding aortic wall distortion (see U.S. Pat. Nos. 6,312,445 and 6,165,186).

In one aspect, automated and modified suturing methods and devices can deliver a surgical fastener (e.g., a suture or suture clip) that comprises an anti-scarring drug combination (or individual component(s) thereof). In another aspect, automated and modified suturing methods and devices can deliver a vascular graft that comprises an anti-scarring drug combination (or individual component(s) thereof) to complete an anastomosis.

(2) Micromechanical devices

Micromechanical devices are used to create an anastomosis and/or secure a graft vessel to the site of an anastomosis. Representative examples of micromechanical devices include staples (either penetrating or non-penetrating) and clips.

Anastomotic staple and clip devices may take a variety of forms and may be made from different types of materials. For example, staples and clips may be formed of a metal or metal alloy, such as titanium, nickel-titanium alloy, or stainless steel, or a polymeric material, such as silicone, poly(urethane), rubber, or a thermoplastic elastomer.

The polymeric material may be an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.

A variety of devices for guiding staples and clips into position also have been described.

One manufacturer of non-penetrating staples for use in the creation of anastomosis is United States Surgical Corp. (Norwalk, Conn.). The VCS system (Autosuture) is an automatic stapling device that applies non-penetrating, titanium vascular clips which are usually used in an interrupted fashion to evert tissue edges with high compressive forces. (See, e.g., U.S. Pat. Nos. 6,440,146, 6,391,039, 6,024,748, 5,833,698, 5,799,857, 5,779,718, 5,725,538, 5,725,537, 5,720,756, 5,360,154, 5,193,731, and 5,005,749 for the description of anastomotic connector devices made by U.S. Surgical).

An anastomotic clip may be composed of a shape memory material, such as nitinol, which is self-closing between an open U-shaped configuration and a closed configuration. See, e.g., U.S. Pat. No. 6,641,593. The anastomotic clip may be composed of a wire having a shape memory that defines a closed configuration which may be substantially spiral-shaped and having a needle that may be releasably attached to the clip. See, e.g., U.S. Pat. No. 6,551,332. Other anastomotic clips are described in, e.g., U.S. Pat. Nos. 6,461,365; and 6,514,265.

Automatic stapling devices are also made by Bypass/Ethicon, Inc. (Somerville, N.J.) and are described in, e.g., U.S. Pat. Nos. 6,193,129; 5,632,433; 5,609,285; 5,533,661; 5,439,156; 5,350,104; 5,333,773; 5,312,024; 5,292,053; 5,285,945; 5,275,322; 5,271,544; 5,271,543 and 5,205,459 and WO 03/02016. Resorbable surgical staples that include a polymer blend that is rich in glycolide (i.e., 65 to 85 weight % polymerized glycolide) are described in, e.g., U.S. Pat. No. 4,741,337 and 4,889,119. Surgical staples made from a blend of lactide/glycolide-copolymer and poly(p-dioxanone) are described in U.S. Pat. No. 4,646,741. Other types of stapling devices are described in, e.g., U.S. Pat. Nos. 5,234,447; 5,904,697 and 6,565,582; and U.S. Publication No. 2002/0185517A1.

In another aspect, the micromechanical device may be an anastomotic clip. For example, an anastomotic clip may be composed of a shape memory material, such as nitinol, which is self-closing between an open U-shaped configuration and a closed configuration. See, e.g., U.S. Pat. No. 6,641,593. The anastomotic clip may be composed of a wire having a shape memory that defines a closed configuration which may be substantially spiral-shaped and having a needle that may be releasably attached to the clip. See, e.g., U.S. Pat. No. 6,551,332. Other anastomotic clips are described in, e.g., U.S. Pat. Nos. 6,461,365; 6,187,019; and 6,514,265.

In one aspect, the present invention provides for the combination of a micromechanical anastomotic device (e.g., a staple or a clip) and an anti-scarring drug combination (or individual component(s) thereof).

(3) Anastomotic Coupling Devices

Anastomotic coupling devices may be used to connect a first blood vessel to a second vessel, either with or without a graft vessel, for completion of an anastomosis. In one aspect, anastomotic coupling devices facilitate automated attachment of a graft or vessel to an aperture or orifice (e.g., in the side or at the end of a vessel) in a target vessel without the use of sutures or staples. In another aspect, the anastomotic coupling device comprises a tubular structure defining a lumen through which blood may flow (described below).

Anastomotic coupling devices that facilitate automated attachment of a graft or vessel to an aperture or orifice in a target vessel may take a variety of forms and may be made from a variety of materials. Typically, such devices are made of a biocompatible material, such as a polymer or a metal or metal alloy. For example, the device may be formed from a synthetic material, such as a fluoropolymer, such as expanded poly(tetrafluoroethylene) (ePTFE) (ePTFE) sold under the trade name GORE-TEX available from W.L. Gore & Associates, Inc. or fluorinated ethylene propylene (FEP), a polyurethane, polyethylene, polyamide (nylon), silicone, polypropylene, polysulfone, or a polyester.

Anastomotic coupling devices may include an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.

The device may include a metal or metal alloy (e.g., nitinol, stainless steel, titanium, iron, nickel, nickel-titanium, cobalt, platinum, tungsten, tantalum, silver, gold, molybdenum, chromium, and chrome), or a combination of a metal and a polymer.

The device may be anchored to the outside of a vessel, within the tissue that surrounds the lumen of a blood vessel, and/or a portion of the device may reside within the lumen of the vessel.

In one aspect, the anastomotic coupler may be an artificially formed aperture connector that is placed in the side wall of the target vessel so that the tubular graft conduit may be extended from the target vessel. The connector may include a plurality of tissue-piercing members and retention fingers disposed in a concentric annular array which may be passed through the side wall of the tubular graft conduit for securing and retaining the graft to the connector in a fluid-tight configuration. See, e.g., U.S. Pat. No. 6,702,829 and 6,699,256.

In another aspect, the anastomotic coupler may be in the form of a frame. For example, the frame may be configured to be deformable and scissor-shaped such that spreading members are moveable to secure a graft vessel upon insertion into a target vessel. See, e.g., U.S. Pat. No. 6,179,849.

In another aspect, the anastomotic coupler may be a ring-like device that is used as an anastomotic interface between a lumen of a graft and an opening in a lumen of a target vessel. For example, the anastomotic ring may be composed of stainless steel alloy, titanium alloy, or cobalt alloy and have a flange with an expandable diameter. See, e.g., U.S. Pat. No. 6,699,257. Anastomosis rings are also described in, e.g., U.S. Pat. No. 6,248,117.

In another aspect, the anastomotic coupler is resorbable. Resorbable anastomotic coupling devices may include, for example, a polymeric blend that is rich in glycolide (i.e., 65 to 85 weight % polymerized glycolide) (see, e.g., U.S. Pat. No. 4,741,337 and 4,889,119) or a blend of lactide/glycolide-copolymer and poly(p-dioxanone) (see, e.g., U.S. Pat. No. 4,646,741).

In another aspect, the anastomotic coupler includes a bioabsorbable, elastomeric material. Representative examples of elastomeric materials for use in resorbable devices are described in, e.g., U.S. Pat. No. 5,468,253.

In another aspect, the anastomotic coupler may be used to connect a first blood vessel to a second vessel, either with or without a graft vessel. For example, the anastomotic coupler may be a device that serves to interconnect two vessels in a side-to-side anastomosis, such as when grafting two juxtaposed cardiac vessels. The anastomotic coupler may be configured as two partially opened cylindrical segments that are interconnected along the periphery by a flow opening whereby the device may be inserted in a minimally-invasive manner which then conforms to provide pressure against the interior wall when in the original configuration such that leakage is prevented. See, e.g., U.S. Pat. Nos. 6,464,709; 6,458,140 and 6,251,116 and U.S. Application Publication No. 2003/0100920A1.

In another aspect, the anastomotic coupler may also be incorporated in the design of a vascular graft to eliminate the step of attaching the interface prior to deployment. For example, the anastomotic coupler may have a leading and rear petal for dilating the vessel opening during advancement, and a base which is configured for attachment to a graft while forming a seal with the opening of the vessel. See, e.g., U.S. Pat. No. 6,702,828.

In another aspect, the anastomotic coupler may be in the form of a frame. For example, the anastomotic coupler may be composed of a deformable, scissor-shaped frame with spreading members that is inserted into a target vessel. See, e.g., U.S. Pat. No. 6,179,849.

In another aspect, the anastomotic coupling device may include a graft that incorporates fixation mechanisms (e.g., a collet or a grommet) at its opposite ends and a heating element to create a thermal bond between the graft and a blood vessel (see, e.g., U.S. Pat. Nos. 6,652,544 and 6,293,955).

In another aspect, the anastomotic coupling device includes a compressible, expandable fitting for securing the ends of a bypass graft to two vessels. The fitting may be incorporated in the bypass graft design to eliminate the step of attaching the graft to the fitting prior to deployment (see, e.g., U.S. Pat. No. 6,494,889).

In another aspect, the anastomotic coupling device includes a pair of coupling disc members for joining two vessels in an end-to-end or end-to-side fashion. One of the members includes hook members, while the other member has receptor cavities aligned with the hooks for locking everted tissue of the vessels together (see, e.g., U.S. Pat. No. 4,523,592).

Representative examples of anastomotic connector devices of Bypass/Ethicon, Inc. are described in U.S. Application Publication Nos. US2002/0082625A1 and 2003/0100910A1 and U.S. Pat. Nos. 6,036,703, 6,036,700, 6,015,416, and 5,346,501.

Other anastomotic coupling devices are those described in e.g., U.S. Pat. No. 6,036,702; 6,508,822; 6,599,303; 6,673,084, 5,695,504; 6,569,173; 4,931,057; 5,868,763; 4,624,257; 4,917,090; 4,917,091; 5,697,943; 5,562,690; 5,454,825; 5,447,514; 5,437,684; 5,376,098; 6,652,542; 6,551,334; and 6,726,694 and U.S. Application Publication Nos. 2003/0120293A1 and 2004/0030348A1.

Anastomotic coupling devices may include proximal aortic connectors and distal coronary connectors. For example, aortic anastomotic connectors include devices such as the SYMMETRY Bypass Aortic Connector device made by St. Jude Medical, Inc. (Maple Grove, Minn.), which consists of an aortic cutter or hole punch assembly and a graft delivery system. The aortic hole punch is a cylindrical cutter with a barbed needle that provides an anchor and back pressure for the rotating cutter to core a round hole in the wall of the aorta. The graft delivery system is a radially expandable nitinol device that holds the vein graft with small hooks which pierce through vein graft wall. The graft is fixed to the aorta through use of an inner and outer ring of struts or flanges. This and other anastomotic connector devices by St. Jude are described in U.S. Pat. Nos. 6,309,416, 6,302,905, 6,152,937, and PCT Publication Nos. WO 00/27312 and WO 00/27311.

The CORLINK Automated Anastomotic connector device, which is produced by the CardioVations division of Ethicon, Inc. (Johnson & Johnson, Somerville, N.J.), uses a nitinol metal alloy fastener to connect the grafted vessel to the aorta. It consists of a central cylindrical body made of interconnected elliptical arches and two sets of several pins radiating from each end. The graft is loaded into a CORLINK insertion instrument and deployed to create an anastomosis in one step.

Further examples of anastomotic coupling devices include those made by Cardica (see, U.S. Pat. Nos. 6,719,769; 6,419,681 and 6,537,287), Converge Medical (formerly Advanced Bypass Technologies), Onux Medical (see, e.g., PCT Publication No. WO 01/34037) and Ventrica, Menlo Park, Calif. (VENTRICA Magnetic Vascular Positioner) (see, e.g., U.S. Pat. Nos. 6,719,768; 6,517,558 and 6,352,543).

As described above, an anastomotic coupling device may comprise a tubular structure defining a lumen through which blood may flow. These types of devices (also referred to herein as “bypass devices”) can function as an artificial passageway or conduit for fluid communication between blood vessels and can be used to divert (i.e., shunt) blood from one part of a blood vessel (e.g., an artery) to another part of the same vessel, or to a second vessel (e.g., an artery or a vein) or to multiple vessels (e.g., a vein and an artery). In one aspect of the invention, the anastomotic device is a bypass device.

Bypass devices may be used in a variety of end-to-end and end-to-side anastomotic procedures. The bypass device may be placed into a patient where it is desired to create a pathway between two or more vascular structures, or between two different parts of the same vascular structure. For example, bypass devices may be used to create a passageway which allows blood to flow around a blood vessel, such as an artery (e.g., coronary artery, carotid artery, or artery supplying the lower limb), which has become damaged or completely or partially obstructed. Bypass devices may be used in coronary artery bypass surgery to shunt blood from an artery, such as the aorta, to a portion of a coronary artery downstream from an occlusion in the artery.

Certain types of anastomotic coupling devices are configured to join two abutting vessels. The device can further include a tubular segment to shunt blood to another vessel. These types of connectors are often used for end-to-end anastomosis if a vessel is severed or injured.

Bypass devices include at least one tubular structure having a first end and a second end, which defines a single lumen through which blood can flow, or may include more than one tubular structure, defining multiple lumens through which blood can flow. The tubular structure includes an extravascular portion and may, optionally, include an intravascular portion. The extravascular portion resides external to the adventitial tissue of a blood vessel, whereas the intravascular portion may reside within the vessel lumen or within the intimal, medial, and/or adventitial tissue.

The configuration of the tubular segment may take a variety of forms. For example, the tubular portion may be generally straight, bent or curved (e.g., L-shaped or helical), tapered, branched (e.g., bifurcated or trifurcated), or may include a network of conduits through which blood may flow. Generally, straight or bent devices have a single lumen through which blood may flow, while branched conduits (e.g., generally T-shaped and Y-shaped devices) and conduit networks (described below) have two or more lumens through which blood may flow. A tubular structure may be in the form, for example, of a hollow cylinder and may or may not include a support structure, such as a mesh or porous framework. Depending on the procedure, the device may be biodegradable or non-biodegradable; expandable or rigid; metal and/or polymeric; and/or may include a shape-memory material (e.g., nitinol). In certain embodiments, the device may include a self-expanding stent structure.

Bypass devices typically are made of a biocompatible material. Any of the materials described above for other types of connectors may be used to make a bypass device, such as a synthetic or naturally-derived polymer, or a metal or metal alloy. For example, the device may be formed from a synthetic material, such as a fluoropolymer, such as expanded poly(tetrafluoroethylene) (ePTFE) or fluorinated ethylene propylene (FEP), a polyurethane, polyethylene, polyamide (nylon), silicone, polypropylene, polysulfone, or a polyester and/or a naturally derived material, such as collagen or a polysaccharide. The device may include a metal or metal alloy (e.g., nitinol, stainless steel, titanium, nickel, nickel-titanium, cobalt, platinum, iron, tungsten, tantalum, silver, gold, molybdenum, chromium and chrome), or a combination of a metal and a polymer. Other types of devices include a natural graft material (e.g., autologous vessel, homologous vessel, or xenograft), or a combination of a synthetic and a natural graft material. In another aspect, the bypass device may be formed of an absorbable or biodegradable material designed to dissolve after completion of the anastomosis (e.g., polylactide, polyglycolide, and copolymers of lactide and glycolide). In yet another aspect, demineralized bone may be used to provide a pliable tubular conduit (see, e.g., U.S. Pat. No. 6,290,718).

The tubular structure(s) include a proximal end that may be configured for attachment to a proximal blood vessel and a distal end configured for attachment to a distal blood vessel. As described above, an anastomosis may be described as being either “proximal” or “distal” depending on its location relative to the vascular obstruction. The “proximal” anastomosis may be formed in a proximal blood vessel, and the “distal” anastomosis may be formed in a distal blood vessel, which may the same vessel or a different vessel than the proximal vessel. The terms “distal” and “proximal” may also be used to describe the direction that blood flows through a tubular structure from one vessel into another vessel. For example, blood may flow from a proximal vessel (e.g., the aorta) into a distal vessel, such as a coronary artery to bypass an obstruction in the coronary artery.

The tubular structure may be attached directly to a proximal or distal blood vessel. Alternatively, the bypass device may further include a graft vessel or be configured to receive a graft vessel, which can be connected to the same or a different blood vessel for completion of the anastomosis. Representative examples of graft vessels include, for example, vascular grafts or grafts used in hemodialysis applications (e.g., AV graft, AV shunt, or AV graft).

In one aspect, a tubular anastomotic coupler includes a proximal end that is attached to a proximal vessel and a distal end that is used to attach a bypass graft. The bypass graft can be secured to the distal vessel to complete the anastomosis. The direction of blood flow can be from the proximal blood vessel and into the proximal end of the tubular structure. Blood can exit through the distal end of the tubular structure and into the graft vessel.

In another aspect, the tubular anastomotic coupler includes a proximal end that is attached to a graft vessel, which is secured to the proximal blood vessel, and a distal end that is configured for attachment to a distal blood vessel. The direction of blood flow can be from the proximal vessel into the graft vessel and into the proximal end of the tubular structure. Blood can exit through the distal end of the tubular structure and into the distal vessel.

Anastomotic bypass devices may be anchored to a blood vessel in a variety of ways and may be attached to a blood vessel for the formation of an anastomosis with or without the use of sutures. Bypass devices may be attached to the outside of a blood vessel, and/or a portion of the device may be implanted into a vessel. For example, a portion of the implanted device may reside within the lumen of the vessel (i.e., endoluminally), and/or a portion of the implanted device may reside intravascularly (i.e., within the intimal, intramural, and/or adventitial tissue of the blood vessel). In one aspect, at least one of the tubular structures, or a portion thereof, may be inserted into the end of a vessel or into the side of a blood vessel. The device may be secured directly to the vessel using, for example, a fastener, such as sutures, staples, or clips and/or an adhesive. Bypass devices may include an interface to secure the conduit to a target vessel without the use of sutures. The interface may include means, such as, for example, hooks, barbs, pins, clamps, or a flange or lip for coupling the device to the site of an anastomosis.

Representative examples of anastomotic coupling devices that include at least one tubular portion include, without limitation, devices used for end-to-end anastomosis procedures (e.g., anastomotic stents and anastomotic sleeves) and end-to-side anastomosis procedures (e.g., single-lumen and multi-lumen bypass devices).

In one aspect of the invention, the anastomotic coupling device comprises a single tubular portion that may by used as a shunt to divert blood from a source vessel to a graft vessel (e.g., in an end-to-side anastomosis procedure). In one aspect, an end of the tubular portion may be connected directly or indirectly to a target vessel, as described above. The opposite end of the tubular portion may be attached to a graft vessel, where the graft vessel may be secured to a target vessel to complete the anastomosis.

The tubular portion(s) may be straight or may have a curved or bent shape (e.g., L-shaped or helical) and may be oriented orthogonally or at an angle relative to the vessel to which it is connected. In one aspect, the conduit may be secured into the site by, for example, a fastener, such as staples, clamps, or hooks, or by adhesives, radiofrequency sealing, or by other methods known to those skilled in the art.

In one aspect, the anastomotic coupling device may be, for example, a tubular metal braided graft with suture rings welded at the distal end to provide a means for securing in place to the target vessel. See, e.g., U.S. Pat. No. 6,235,054. Other types of conduits that are secured into the site include, e.g., U.S. Pat. Nos. 4,368,736 and 4,366,819.

In certain types of single-lumen coupling devices, the conduit terminates in a flange that resides within the lumen of the vessel. For example, the conduit may have a tubular body with a connector which has a plurality of extensions and is configured for disposition annularly within the inside of a tubular vessel. See, e.g., U.S. Pat. No. 6,660,015. In other devices, the flange may be attached into or onto the surface of the adventitial tissue of the blood vessel.

Other types of single-lumen bypass devices are described, for example, in U.S. Pat. Nos. 6,241,743; 6,428,550; 6,241,743; 6,428,550; 5,904,697; 5,290,298; 6,007,576; 6,361,559; 6,648,901, 4,931,057 and U.S. Application Publication Nos. 2004/0015180A1, 2003/0065344A1, and 2002/0116018A1.

In one aspect of the invention, the anastomotic coupling device comprises more than one lumen through which blood may travel. Multi-lumen bypass devices may include two or more tubular portions configured to interconnect multiple (two or more) blood vessels. Multi-lumen coupling devices may be used in a variety of anastomosis procedures. For example, such devices may be used in coronary artery bypass graft (CABG) surgery to divert blood from an occluded proximal vessel (e.g., an artery) into one or more target (i.e., distal) vessels (e.g., an artery or vein).

In one aspect, at least one tubular portion may by used as a shunt for diverting blood between a source vessel and a target vessel. In another aspect, the device may be configured as an interface for securing a graft vessel to a target vessel for completion of an anastomosis. Depending on the procedure, the tubular arms may be of equal length and diameter or of unequal length and diameter and may include a tubular portion(s) that is expandable and/or includes a shape-memory material (e.g., nitinol). Furthermore, the tubular portions may be made of the same material or a different material.

In one aspect, one or more ends of a tubular portion may be inserted into the end or into the side of one or more blood vessels. In other embodiments, one or more tubular portions of the device may reside within the lumen of a blood or graft vessel. The device, optionally, may be secured to the blood vessel using a fastener or an adhesive, or another approach known to those skilled in the art.

At least one arm of the multi-lumen connector may be attached to a graft vessel. The graft vessel may be a synthetic graft, such as an ePTFE or polyester graft, or natural graft material (e.g., autologous vessel, homologous vessel, or xenograft), or a combination of a synthetic and a natural graft material. In certain embodiments, a graft vessel may be attached to an end of a tubular portion of the device, and a second graft vessel may be attached to the opposite end of the same tubular portion or to the end of another tubular portion. The graft vessel(s) may be further attached to a target vessel(s) for the completion of the anastomosis.

In one aspect, the device may include three or more tubular arms that extend from a junction site. For example, the multi-lumen device may be generally T-shaped or Y-shaped (i.e., having two or three lumens, respectively). For example, the multi-lumen device may be a T-shaped tubular graft connector having a longitudinal member that extends into the target vessel and a second section that is exterior to the vessel which provides a connection to an alternate tubular structure. See, e.g., U.S. Pat. Nos. 6,152,945 and 5,972,017. Other multi-lumen devices are described in, (see, e.g., U.S. Pat. Nos. 6,152,945; 6,451,033; 5,755,778; 5,922,022; 6,293,965; 6,517,558 and 6,626,914 and U.S. Publication No. 2004/0015180A1).

In another aspect, the device may be a tube for bypassing blood flow directly from a portion of the heart (e.g., left ventricle) to a coronary artery. For example, the device may be a hollow tube that may be partially closable by a one-way valve in response to movement of the cardiac tissue during diastole while permitting blood flow during systole (see, e.g., U.S. Pat. No. 6,641,610). The device may be an elongated rigid shunt body composed of a diversion tube having two apertures in which one may be disposed within the cyocardium of the left ventricle and the other may be disposed within the coronary artery (see, e.g., WO 00/15146 and U.S. Application Publication No. 2003/0055371A1). The device may be a valved, tubular apparatus that is L- or T-shaped which is adapted for insertion into the wall of the heart to provide blood communication from the heart to a coronary vessel (see, e.g., U.S. Pat. No. 6,123,682).

In another aspect, the device may include a network of interconnected tubular conduits. For example, the device may include two tubular portions that may be oriented generally axially or orthogonally relative to each other. See U.S. Pat. No. 6,241,761 and 6,241,764. Communication between the two tubular structures may be achieved through a flow channel which facilitates blood to flow between the bores of each tube.

In another aspect, the anastomotic coupling device is a resorbable device that may be configured with two or three termini which provide a vessel interface without the need for sutures and provides a fluid communication through an intersecting lumen, such as a bypass graft or alternate vessel. See, e.g., U.S. Application Publication Nos. 2002/0052572A1 and PCT Publication No. WO 02/24114A2. An anastomotic connector may also be formed of a resorbable tubular structure configured to include snap-connectors or other components for securing it to the tissue as well as hemostasis inducing sealing rings to prevent blood leakage. See, e.g., U.S. Pat. Nos. 6,056,762. The anastomotic connector may be designed with three legs whereby two legs are adapted to be inserted within the continuous blood vessel in a contracted state and then enlarged to form a tight fit and the third leg is adapted for connecting and sealing with a third conduit. See, e.g., U.S. Pat. No. 6,019,788.

An example of a commercially available multi-lumen anastomotic coupling device is the SOLEM graft connector (made by Jomed, Sweden). This device, which is described in more detail in PCT Publication No. WO 01/13820, and U.S. Pat. Nos. 6,179,848, D438618 and D429334, includes a T-shaped connector composed of nitinol and an ePTFE graft for completion of a distal anastomosis.

Another example of an anastomotic connector is the HOLLY GRAFT System (in development) for use in bypass surgery from CABG Medical, Inc. (Minneapolis, Minn.), which is described, e.g., in U.S. Pat. Nos. 6,241,761 and 6,241,764.

In one aspect, the present invention provides for the combination of an anastomotic coupling device and an anti-scarring drug combination (or individual component(s) thereof)or a composition comprising an anti-scarring drug combination (or individual component(s) thereof). In one aspect, the anastomotic coupling device may be attached to a blood vessel for the formation of an anastomosis without the use of sutures or staples. In certain aspects, the anastomotic coupling device may comprise a tubular structure defining a lumen through which blood may flow, and an anti-scarring drug combination (or individual component(s) thereof). The device may include one, two, three, or more lumens defined by one, two, three, or more tubular structures, depending on the number of vessels to be connected.

Introduction of an anastomotic connector into or onto an intramural, luminal, or adventitial portion of a blood vessel may irritate or damage the endothelial tissue of the blood vessel and/or may alter the natural hemodynamic flow through the vessel. This irritation or damage may stimulate a cascade of biological events resulting in a fibrotic response which can lead to the formation of scar tissue in the vessel. Incorporation of a therapeutic agent in accordance with the invention into or onto a portion of the device that is in direct contact with the blood vessel (e.g., a terminal portion or edge of the device) may inhibit one or more of the scarring processes described above (e.g., smooth muscle cell proliferation, cell migration, inflammation), making the vessel less prone to the formation of intimal hyperplasia and stenosis.

Thus, in one aspect, the therapeutic agent may be associated only with the portion of the device that is in contact with the blood or endothelial tissue. For example, the anti-scarring drug combination (or individual component(s) thereof) may be incorporated into only an intravascular portion (i.e., that portion that resides within the lumen of the vessel or in the vessel tissue) of the device. The anti-scarring drug combination (or individual component(s) thereof) may be incorporated onto all or a portion of the intravascular portion of the device. In other embodiments, the coating may reside on all or a portion of an extravascular portion of the device.

The anti-scarring drug combination (or individual component(s) thereof) or a composition that includes an anti-scarring drug combination (or individual component(s) thereof) may be coated onto a portion of or onto the entire surface of the device or may be incorporated into a portion of, or into the entire the structure of, the device (e.g., either within voids, reservoirs, or divets in the device or within the material used to construct the device). In other aspects, the agent or a composition comprising the agent is impregnated into or affixed onto the device surface.

As described above, the device may include a tubular portion that is disposed within the lumen of a blood vessel. The entire tubular portion may, for example, be coated with an anti-scarring drug combination (or individual component(s) thereof) or a composition comprising an anti-scarring drug combination (or individual component(s) thereof). Alternatively, only a portion of the tubular portion may include the anti-scarring drug combination (or individual component(s) thereof). For example, only an external (abluminal) surface or only the interior (endoluminal) surface of the tubular portion may be coated. In other embodiments, one or both termini of the tubular portion may be coated. For example, the endoluminal and/or abluminal surface of the tubular section through which blood enters into the device (i.e., proximal end) may be coated with the anti-scarring drug combination (or individual component(s) thereof) or composition comprising the anti-scarring drug combination (or individual component(s) thereof). In another aspect, the endoluminal and/or abluminal surface of the tubular section through which blood exits (i.e., distal end) from the device may be coated with the anti-scarring drug combination (or individual component(s) thereof) or composition comprising the anti-scarring drug combination (or individual component(s) thereof).

In another embodiment, the anti-scarring drug combination (or individual component(s) thereof) or composition comprising the anti-scarring drug combination (or individual component(s) thereof) is associated (e.g., coated onto or incorporated into) with an anchoring member (e.g., a fastener, such as a staple or clip) that secures the device to a blood vessel.

As described above, anastomotic connector devices can include a fibrosis-inhibiting drug combination (or individual component(s) thereof) as a means to improve the clinical efficacy of the device. In another approach, the fibrosis-inhibiting drug combination (or individual component(s) thereof) can be incorporated into or onto a film or mesh (described in further detail below) that is applied in a perivascular manner to an anastomotic site (e.g., at the junction of a graft vessel and the blood vessel). These films or wraps can be used with any of the anastomotic connector devices described above and, typically, are placed around the outside of the anastomosis at the time of surgery. In other embodiments, the agent drug combination (or individual component(s) thereof) may be delivered to the anastomotic site in the form of a spray, paste, gel, or the like. In yet another approach, the fibrosis-inhibiting drug combination (or individual component(s) thereof) can be incorporated into or onto the graft vessel that is secured to the blood vessel with the connector device.

In yet another aspect, other specialized intravascular devices, such as coronary drug infusion guidewires, such as those available from TherOx, Inc., grafts and balloon over stent devices, such as are described in Wilensky, R. L. (1993) J. Am. Coll. Cardiol.: 21: 185A can also be utilized for local delivery of an anti-fibrosing drug combination (or individual component(s) thereof).

As described above, the present invention provides intravascular devices (e.g., anastomotic connectors, stents, drug-delivery balloons, intravascular catheters) that include an anti-scarring drug combination (or individual component(s) thereof) or a composition that includes an anti-scarring drug combination (or individual component(s) thereof). Numerous polymeric and non-polymeric delivery systems for use with intravascular devices have been described above. Methods for incorporating coating fibrosis-inhibiting drug combinations (or individual components thereof) and compositions onto or into intravascular devices include: (a) directly affixing to the intravascular device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) by inserting the device into a sleeve or mesh which contains or is coated with a fibrosis-inhibiting drug combination (or individual component(s) thereof) or a composition comprising a fibrosis-inhibiting drug combination (or individual component(s) thereof), (f) constructing the device itself or a portion of the device with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. For these devices, the coating process can be performed in such a manner as to (a) coat the external surface of the stent, (b) coat the internal (luminal) surface of the stent or (c) coat all or parts of both the internal and external surfaces of the stent.

The intravascular device (e.g., a stent) may be adapted to release the desired drug combination (or individual component(s) thereof) at only the distal ends, or along the entire body of the device.

According to the present invention, any fibrosis-inhibiting drug combination (or individual component(s) thereof) described above can be utilized in the practice of this embodiment. Within one embodiment of the invention, intravascular devices may be adapted to release a drug combination (or individual component(s) thereof) that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As intravascular devices are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of drug combinations for use in intravascular devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the intravascular device, the exemplary anti-fibrosing drug combination (or individual component(s) thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with intravascular devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Gastrointestinal Stents

The present invention provides for the combination of an anti-fibrosis drug combination (or individual component(s) thereof) and a gastrointestinal (GI) stent. The term “GI stent” refers to devices that are located in the gastrointestinal tract including the biliary duct, pancreatic duct, colon, and the esophagus. GI stents are or comprise scaffoldings that are used to treat endoluminal body passageways that have become blocked due to disease or damage, including malignancy or benign disease.

In one aspect, the GI stent may be an esophageal stent used to keep the esophagus open whereby food is able to travel from the mouth to the stomach. For example, the esophageal stent may be composed of a cylindrical supporting mesh inner layer, retaining mesh outer layer and a semi-permeable membrane sandwiched between. See, e.g., U.S. Pat. No. 6,146,416. The esophageal stent may be a radially, self-expanding stent of open weave construction with an elastomeric film formed along the stent to prevent tissue ingrowth and distal cuffs that resist stent migration. See, e.g., U.S. Pat. No. 5,876,448. The esophageal stent may be composed of a flexible wire configuration to form a cylindrical tube with a deformed end portion increased to a larger diameter for anchoring pressure. See, e.g., U.S. Pat. No. 5,876,445. The esophageal stent may be a flexible, self-expandable tubular wall incorporating at least one truncated conical segment along the longitudinal axis. See, e.g. U.S. Pat. No. 6,533,810.

In another aspect, the GI stent may be a biliary stent used to keep the biliary duct open whereby bile is able to drain into the small intestines. For example, the biliary stent may be composed of shape memory alloy. See, e.g., U.S. Pat. No. 5,466,242. The biliary stent may be a plurality of radially extending wings with grooves which project from a helical core. See, e.g., U.S. Pat. Nos. 5,776,160 and 5,486,191.

In another aspect, the GI stent may be a colonic stent. For example, the colonic stent may be a hollow tubular body that may expand radially and be secured to the inner wall of the organ in a release fitting. See, e.g., European Patent Application No. EP 1092400A2.

In another aspect, the GI stent may be a pancreatic stent used to keep the pancreatic duct open to facilitate secretion into the small intestines. For example, the pancreatic stent may be composed of a soft biocompatible material which is resiliently compliant which conforms to the duct's curvature and contains perforations that facilitates drainage. See, e.g., U.S. Pat. No. 6,132,471.

GI stents, which may be combined with one or more drugs according to the present invention, include commercially available products, such as the NIR Biliary Stent System and the WALLSTENT Endoprostheses from Boston Scientific Corporation.

In one aspect, the present invention provides GI stents that include an anti-scarring drug combination (or individual component(s) thereof) or a composition that includes an anti-scarring drug combination (or individual component(s) thereof). Numerous polymeric and non-polymeric delivery systems for use in GI stents have been described above.

Methods for incorporating fibrosis-inhibiting drug combination (or individual component(s) thereof) or fibrosis-inhibiting compositions onto or into the GI stents include: (a) directly affixing to the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the stent with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the stent structure, (e) by inserting the stent into a sleeve or mesh which is comprised of or coated with a fibrosis-inhibiting drug combination (or individual component(s) thereof) or a composition comprising a fibrosis-inhibiting drug combination (or individual component(s) thereof), (f) constructing the stent itself or a portion of the stent with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the stent surface or to a linker (small molecule or polymer) that is coated or attached to the stent surface. For these devices, the coating process can be performed in such a manner as to (a) coat the external surface of the stent, (b) coat the internal (luminal) surface of the stent or (c) coat all or parts of both the internal and external surfaces of the stent.

In addition to coating the GI stent with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device. This can include the GI stent structure itself, the outer covering or sleeve, if applicable, or both the stent structure and the outer covering or sleeve.

According to the present invention, any fibrosis-inhibiting drug combination (or individual component(s) thereof) described above can be utilized in the practice of this embodiment. Within one embodiment of the invention, GI stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As GI stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of anti-scarring drug combinations for use in GI stents include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the GI stent, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with GI stent devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred lug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Tracheal and Bronchial Stents

The present invention provides for the combination of an anti-scarring drug combination (or individual component(s) thereof) and a tracheal or bronchial stent device.

Representative examples of tracheal or bronchial stents that can benefit from being coated with or having incorporated therein, a fibrosis-inhibiting drug combination (or individual component(s) thereof) include tracheal stents or bronchial stents, including metallic and polymeric tracheal or bronchial stents and tracheal or bronchial stents that have an external covering (e.g., polyurethane, poly(ethylene terephthalate), PTFE, or silicone rubber).

Tracheal and bronchial stents may be, for example, composed of an elastic plastic shaft with metal clasps that expands to form a lumen along the axis for opening the diseased portion of the trachea and having three sections to emulate the natural shape of the trachea. See, e.g., U.S. Pat. No. 5,480,431. The tracheal/bronchial stent may be a T-shaped tube having a tracheotomy tubular portion that projects outwardly through a tracheotomy orifice which is configured to close and form a fluid seal. See, e.g., U.S. Pat. Nos. 5,184,610 and 3,721,233. The tracheal/bronchial stent may be composed of a flexible, synthetic polymeric resin with a tracheotomy tube mounted on the wall with a bifurcated bronchial end that is configured in a T-Y shape with specific curves at the intersections to minimize tissue damage. See, e.g., U.S. Pat. No. 4,795,465. The tracheal/bronchial stent may be a scaffolding configured to be substantially cylindrical with a shape-memory frame having geometrical patterns and having a coating of sufficient thickness to prevent epithelialization. See, e.g., U.S. Patent Application Publication No. 2003/0024534A1.

Tracheal/bronchial stents, which may be combined with one or more agents according to the present invention, include commercially available products, such as the WALLSTENT Tracheobronchial Endoprostheses and ULTRAFLEX Tracheobronchial Stent Systems from Boston Scientific Corporation and the DUMON Tracheobronchial Silicone Stents from Bryan Corporation (Woburn, Mass.).

In one aspect, the present invention provides tracheal and bronchial stents that include an anti-scarring drug combination (or individual component(s) thereof) or a composition that includes an anti-scarring drug combination (or individual component(s) thereof). Numerous polymeric and non-polymeric delivery systems for use in tracheal and bronchial stents have been described above. Methods for incorporating fibrosis-inhibiting drug combination (or individual component(s) thereof) or fibrosis-inhibiting compositions onto or into the tracheal or bronchial stents include: (a) directly affixing to the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier (c) by coating the stent with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the stent structure, (e) by inserting the stent into a sleeve or mesh which is comprised of or coated with a fibrosis-inhibiting drug combination (or individual component(s) thereof) or a composition comprising a fibrosis-inhibiting drug combination (or individual component(s) thereof), (f) constructing the stent itself or a portion of the stent with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the stent surface or to a linker (small molecule or polymer) that is coated or attached to the stent surface. For these devices, the coating process can be performed in such a manner as to (a) coat the external surface of the stent, (b) coat the internal (luminal) surface of the stent or (c) coat all or parts of both the internal and external surfaces of the stent.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device. This can include the stent structure itself, the outer covering or sleeve, if applicable, or both the stent structure and the outer covering or sleeve.

According to the present invention, any fibrosis-inhibiting drug combination (or individual component(s) thereof) described above can be utilized in the practice of this embodiment. Within one embodiment of the invention, tracheal and bronchial stents may be adapted to release an drug combination (or individual component(s) thereof) that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As tracheal and bronchial stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several fibrosis-inhibiting drug combination (or individual component(s) thereof) for use in tracheal and bronchial stents include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the tracheal or bronchial stent, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with tracheal and bronchial stent devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Genital-Urinary Stents

The present invention provides for the combination of an anti-scarring drug combination (or individual component(s) thereof) and genital-urinary (GU) stent device.

Representative examples genital-urinary (GU) stents that can benefit from being coated with or having incorporated therein, a fibrosis-inhibiting drug combination (or individual component(s) thereof) include ureteric and urethral stents, fallopian tube stents, prostate stents, including metallic and polymeric GU stents and GU stents that have an external covering (e.g., polyurethane, poly(ethylene terephthalate), PTFE or silicone rubber).

In one aspect, genital-urinary stents include ureteric and urethral stents. Ureteral stents are hollow tubes with holes along the sides and coils at either end to prevent migration. Ureteral stents are used to relieve obstructions (caused by stones or malignancy), to facilitate the passage of stones, or to allow healing of ureteral anastomoses or leaks following surgery or trauma. They are placed endoscopically via the bladder or percutaneously via the kidney.

Urethral stents are used for the treatment of recurrent urethral strictures, detruso-external sphincter dyssynergia and bladder outlet obstruction due to benign prostatic hypertrophy. In addition, procedures that are conducted for the prostate, such as external radiation or brachytherapy, may lead to fibrosis due to tissue insult resulting from these procedures. The incidence of urethral stricture in prostate cancer patients treated with external beam radiation is about 2%. Development of urethral stricture may also occur in other conditions such as following urinary catheterization or surgery, which results in damage to the epithelium of the urethra. The clinical manifestation of urinary tract obstruction includes decreased force and caliber of the urinary stream, intermittency, postvoid dribbling, hesitance and nocturia. Complete closure of the urethra can result in numerous problems including eventual kidney failure. To maintain patency in the urethra, urethral stents may be used. The stents are typically self-expanding and composed of metal superalloy, titanium, stainless steel or polyurethane.

For example, the ureteric/urethral stent may be composed of a main catheter body of flexible polymeric material having an enlarged entry end with a hydrophilic tip that dissolves when contacted with body fluids. See, e.g., U.S. Pat. No. 5,401,257. The ureteric/urethral stent may be composed of a multi-sections including a closed section at that the bladder end which does not contain any fluid passageways such that it acts as an anti-reflux device to prevent reflux of urine back into the kidney. See, e.g., U.S. Pat. No. 5,647,843. The ureteric/urethral stent may be composed of a central catheter tube made of shape memory material that forms a stent with a retention coil for anchoring to the ureter. See, e.g., U.S. Pat. No. 5,681,274. The ureteric/urethral stent may be a composed of an elongated flexible tubular stent with preformed set curls at both ends and an elongated tubular rigid extension attached to the distal end which allows the combination function as an externalized ureteral catheter. See, e.g., U.S. Pat. Nos. 5,221,253 and 5,116,309. The ureteric/urethral stent may be composed of an elongated member, a proximal retention structure, and a resilient portion connecting them together, whereby they are all in fluid communication with each other with a slideable portion providing a retracted and expanded position. See, e.g., U.S. Pat. No. 6,685,744. The ureteric/urethral stent may be a hollow cylindrical tube that has a flexible connecting means and locating means that expands and selectively contracts. See, e.g., U.S. Pat. No. 5,322,501. The ureteric/urethral stent may be composed of a stiff polymeric body that affords superior columnar and axial strength for advancement into the ureter, and a softer bladder coil portion for reducing the risk of irritation. See, e.g., U.S. Pat. No. 5,141,502. The ureteric/urethral stent may be composed of an elongated tubular segment that has a pliable wall at the proximal region and a plurality of members that prevent blockage of fluid drainage upon compression. See, e.g., U.S. Pat. No. 6,676,623. The ureteric/urethral stent may be a catheter composed of a conduit which is part of an assembly that allows for non-contaminated insertion into a urinary canal by providing a sealing member that surrounds the catheter during dismantling. See, e.g., U.S. Patent Application Publication No. 2003/0060807A1.

In another aspect, genital-urinary stents include prostatic stents. For example, the prostatic stent may be composed of two polymeric rings constructed of tubing with a plurality of connecting arm members connecting the rings in a parallel manner. See, e.g., U.S. Pat. No. 5,269,802. The prostatic stent may be composed of thermoplastic material and a circumferential reinforcing helical spring, which provides rigid mechanical support while being flexible to accommodate the natural anatomical bend of the prostatic urethra. See, e.g., U.S. Pat. No. 5,069,169.

In another aspect, genital-urinary stents include fallopian stents and other female genital-urinary devices. For example, the genital-urinary device may be a female urinary incontinence device composed of a vaginal-insertable supporting portion that is resilient and flexible, which is capable of self-support by expansion against the vaginal wall and extending about the urethral orifice. See, e.g., U.S. Pat. No. 3,661,155. The genital-urinary device may be a urinary evacuation device composed of a ovular bulbous concave wall having an opening to a body engaging perimetal edge integral with the wall and an attached tubular member with a pleated body. See, e.g., U.S. Pat. No. 6,041,448.

Genital-urinary stents, which may be combined with one or more agents according to the present invention, include commercially available products, such as the UROLUME Endoprosthesis Stents from American Medical Systems, Inc. (Minnetonka, Minn.), the RELIEVE Prostatic/Urethral Endoscopic Device from InjecTx, Inc. (San Jose, Calif.), the PERCUFLEX Ureteral Stents from Boston Scientific Corporation, and the TARKINGTON Urethral Stents and FIRLIT-KLUGE Urethral Stents from Cook Group Inc (Bloomington, Ind.).

In one aspect, the present invention provides GU stents that include an anti-scarring drug combination (or individual component(s) thereof) or a composition that includes an anti-scarring drug combination (or individual component(s) thereof). Numerous polymeric and non-polymeric delivery systems for use in GU stents have been described above. Methods for incorporating fibrosing drug combinations (or individual components thereof) or fibrosis-inhibiting compositions onto or into the GU stents include: (a) directly affixing to the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the stent with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the stent structure, (e) by inserting the stent into a sleeve or mesh which is comprised of or coated with a fibrosis-inhibiting drug combination (or individual component(s) thereof) or a composition comprising a fibrosis-inhibiting drug combination (or individual component(s) thereof), (f) constructing the stent itself or a portion of the stent with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the stent surface or to a linker (small molecule or polymer) that is coated or attached to the stent surface. For these devices, the coating process can be performed in such a manner as to (a) coat the external surface of the stent, (b) coat the internal (luminal) surface of the stent or (c) coat all or parts of both the internal and external surfaces of the stent.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting agent is incorporated into the final device.

According to the present invention, any fibrosis-inhibiting drug combination (or individual component(s) thereof) described above can be utilized in the practice of this embodiment. Within one embodiment of the invention, GU stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As GU stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of scarring agents for use in GU stents include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the GU stent, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combination (or individual component(s) thereof) that can be used in conjunction with GU stent devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15,1:20, 1:30, 1:50, 1:75, 1:100, 1:200, 1:500, and 1:1000.

Ear and Nose Stents

The present invention provides for the combination of an anti-scarring drug combination (or individual component(s) thereof) and an ear-nose-throat (ENT) stent device (e.g., a lacrimal duct stent, Eustachian tube stent, nasal stent, or sinus stent).

The sinuses are four pairs of hollow regions contained in the bones of the skull named after the bones in which they are located (ethmoid, maxillary, frontal and sphenoid). All are lined by respiratory mucosa which is directly attached to the bone. Following an inflammatory insult such as an upper respiratory tract infection or allergic rhinitis, a purulent form of sinusitis can develop. Occasionally secretions can be retained in the sinus due to altered ciliary function or obstruction of the opening (ostea) that drains the sinus. Incomplete drainage makes the sinus prone to infection typically with Haemophilus influenza, Streptococcus pneumoniae, Moraxella catarrhalis, Veillonella, Peptococcus, Corynebacterium acnes and certain species of fungi.

When initial treatment such as antibiotics, intranasal steroid sprays and decongestants are ineffective, it may become necessary to perform surgical drainage of the infected sinus. Surgical therapy often involves debridement of the ostea to remove anatomic obstructions and removal of parts of the mucosa. Occasionally a stent (a cylindrical tube which physically holds the lumen of the ostea open) is left in the osta to ensure drainage is maintained even in the presence of postoperative swelling. ENT stents, typically made of stainless steel or plastic, remain in place for several days or several weeks before being removed.

Representative examples of ENT stents that can benefit from being coated with or having incorporated therein a fibrosis-inhibiting drug combination (or individual component(s) thereof) include lacrimal duct stents, Eustachian tube stents, nasal stents, and sinus stents.

In one aspect, the present invention provides for the combination of a lacrimal duct stent and a fibrosis-inhibiting drug combination (or individual component(s) thereof) or a composition comprising a fibrosis-inhibiting drug combination (or individual component(s) thereof).

In another aspect, the present invention provides for the combination of a Eustachian tube stent and a fibrosis-inhibiting drug combination (or individual component(s) thereof) or a composition comprising a fibrosis-inhibiting drug combination (or individual component(s) thereof).

In yet another aspect, the present invention provides for the combination of a sinus stent and a fibrosis-inhibiting drug combination (or individual component(s) thereof) or a composition comprising a fibrosis-inhibiting drug combination (or individual component(s) thereof).

In yet another aspect, the present invention provides for the combination of a nasal stent and a fibrosis-inhibiting drug combination (or individual component(s) thereof) or a composition comprising a fibrosis-inhibiting drug combination (or individual component(s) thereof).

The ENT stent may be a choanal atresia stent composed of two long hollow tubes that are bridged by a flexible transverse tube. See, e.g., U.S. Pat. No. 6,606,995. The ENT stent may be an expandable nasal stent for postoperative nasal packing composed of a highly porous, pliable and absorbent foam material capable of expanding outwardly, which has a nonadherent surface. See, e.g., U.S. Pat. No. 5,336,163. The ENT stent may be a nasal stent composed of a deformable cylinder with a breathing passageway that has a smooth outer non-absorbent surface used for packing the nasal cavity following surgery. See, e.g., U.S. Pat. No. 5,601,594. The ENT stent may be a ventilation tube composed of a flexible, plastic, tubular vent with a rectangular flexible flange which is used for the nasal sinuses following endoscopic antrostomy. See, e.g., U.S. Pat. No. 5,246,455. The ENT stent may be a ventilating ear tube composed of a shaft and an extended tab which is used for equalizing the pressure between the middle ear and outer ear. See, e.g., U.S. Pat. No. 6,042,574. The ENT stent may be a middle ear vent tube composed of a non-compressible, tubular base and an eccentric flange. See, e.g., U.S. Pat. No. 5,047,053.

ENT stents, which may be combined with one or more agents according to the present invention, include commercially available products such as Genzyme Corporation (Ridgefield, N.J.) SEPRAGEL Sinus Stents and MEROGEL Nasal Dressing and Sinus Stents from Medtronic Xomed Surgical Products, Inc. (Jacksonville, Fla.).

In one aspect, the present invention provides ENT stents that include an anti-scarring drug combination (or individual component(s) thereof) or a composition that includes an anti-scarring drug combination (or individual component(s) thereof). Numerous polymeric and non-polymeric delivery systems for use in ENT stents have been described above. Methods for incorporating fibrosis-inhibiting drug combinations (or individual components thereof) or compositions onto or into the ENT stents include: (a) directly affixing to the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the stent a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the stent with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the stent structure, (e) by inserting the stent into a sleeve or mesh which is comprised of or coated with a fibrosis-inhibiting drug combination (or individual component(s) thereof) or a composition comprising a fibrosis-inhibiting drug combination (or individual component(s) thereof), (f) constructing the stent itself or a portion of the stent with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the stent surface or to a linker (small molecule or polymer) that is coated or attached to the stent surface. For these devices, the coating process can be performed in such a manner as to (a) coat the external surface of the specific stent, (b) coat the internal (luminal) surface of the stent, or (c) coat all or parts of both the internal and external surfaces of the device.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device.

According to the present invention, any fibrosis-inhibiting drug combination (or individual component(s) thereof) described above can be utilized in the practice of this embodiment. Within one embodiment of the invention, ENT stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As ENT stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting agents for use in ENT stents include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the ENT stent, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with ENT stent devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Ear Ventilation Tubes

In another aspect, the present invention provides for the combination of an anti-scarring drug combination (or individual component(s) thereof) and an ear ventilation tube (also referred to as a tympanostomy tube).

Acute otitis media is the most common bacterial infection, the most frequent indication for surgical therapy, the leading cause of hearing loss and a common cause of impaired language development in children. The cost of treating this condition in children under the age of five is estimated at $5 billion annually in the United States alone. In fact, 85% of all children will have at least one episode of otitis media and 600,000 will require surgical therapy annually. The prevalence of otitis media is increasing and for severe cases surgical therapy is more cost effective than conservative management.

Acute otitis media (bacterial infection of the middle ear) is characterized by Eustachian tube dysfunction leading to failure of the middle ear clearance mechanism. The most common causes of otitis media are Streptococcus pneumoniae (30%), Haemophilus influenza (20%), Branhamella catarrhalis (12%), Streptococcus pyogenes (3%), and Staphylococcus aureus (1.5%). The end result is the accumulation of bacteria, white blood cells and fluid which, in the absence of an ability to drain through the Eustachian tube, results in increased pressure in the middle ear. For many cases antibiotic therapy is sufficient treatment and the condition resolves. However, for a significant number of patients the condition becomes frequently recurrent or does not resolve completely. In recurrent otitis media or chronic otitis media with effusion, there is a continuous build-up of fluid and bacteria that creates a pressure gradient across the tympanic membrane causing pain and impaired hearing. Fenestration of the tympanic membrane (typically with placement of a tympanostomy tube) relieves the pressure gradient and facilitates drainage of the middle ear (through the outer ear instead of through the Eustachian tube—a form of “Eustachian tube bypass”).

Recurrent otitis media or otitis media with effusion may be treated with tympanostomy tubes or artificial Eustachian tubes/stents, such as described above. These ventilation tubes are indicated for chronic otitis media with effusion, recurrent acute otitis media, tympanic membrane atelectasis, and complications of acute otitis media in children. The excessive formation of granulation tissue around these devices can result in a decreased functioning of these devices. This can then result in a second procedure to either clear the obstruction or to insert a new device. The incorporation of a fibrosis-inhibiting agent into or onto the ventilation tubes may prevent the overgrowth of this granulation tissue.

Surgical placement of tympanostomy tubes is the most widely used treatment for chronic otitis media because, although not curative, it improves hearing (which in turn improves language development) and reduces the incidence of acute otitis media. Tympanostomy tube placement is one of the most common surgical procedures in the United States with 1.3 million surgical placements per year.

Representative examples of ear ventilation tubes that can benefit from being coated with or having incorporated therein a fibrosis-inhibiting agent include, without limitation, grommet-shaped tubes, T-tubes, tympanostomy tubes, drain tubes, tympanic tubes, otological tubes, myringotomy tubes, artificial Eustachian tubes, Eustachian tube prostheses, and Eustachian stents. Ear ventilation tubes have been made out of, e.g., polytetrafluoroethylene (e.g., TEFLON), silicone, nylon, polyethylene and other polymers, stainless steel, titanium, and gold plated steel.

In one aspect, the ear ventilation tube may be a tympanostomy tube that is used to provide an alternative conduit for ventilation of the middle ear cavity via the external ear canal. Typically, ventilation of the middle ear is performed by conducting a myringotomy, in which a slit or opening in the tympanic membrane is surgically made to alleviate a buildup or reduction of pressure in the middle ear cavity and to drain accumulated fluids. Tympanostomy tubes may be inserted into the surgical slit of the tympanic membrane to serve as a bypass for the normal Eustachian tube, which drains the middle ear cavity under normal conditions. For example, the tympanostomy tube may be an elongated uniform tubular member composed of pure titanium or titanium alloy that has a concavity inwardly spaced from one end that forms a flange. See, e.g., U.S. Pat. No. 5,645,584. The tympanostomy tube may be composed of a micro-pitted titanium exterior flangeless surface used to ventilate the middle ear. See, e.g., U.S. Pat. No. 4,971,076. The tympanostomy tube may be composed of a shaft with a tab that extends outwardly perpendicular from the bottom of the shaft. See, e.g., U.S. Pat. No. 6,042,574. The tympanostomy tube may be a permanent ear ventilation device composed of an elongated tubular base having a flange eccentrically connected made of a non-compressible material. See, e.g., U.S. Pat. No. 5,047,053. The tympanostomy tube may be composed of a cap-plug, central body and end cap, which together form a plurality of lumens within the tube. See, e.g., U.S. Pat. No. 5,851,199. The tympanostomy tube may be composed of a microporous resin cured to form a gas-permeable matrix containing a homogenous dispersion of silver particles capable of migrating to the surface of the tube sidewalls to provide antimicrobial activity. See, e.g., U.S. Pat. No. 6,361,526. The tympanostomy tube may be composed of tubular body and a rib structure that projects outwardly to define a channel spiraling around the tubular body. See, e.g., U.S. Pat. No. 5,775,336. The tympanostomy tube may be composed of an integral cutting tang extending from one of two flanges of a grommet for incising the tympanic membrane. See, e.g., U.S. Pat. Nos. 5,827,295 and 5,643,280. The tympanostomy tube may be composed of a tubular member having two opposed flanges in which the insertion of the tube is facilitated by a cutting edge on the flange which induces an incision of the tympanic membrane. See, e.g., U.S. Pat. Nos. 5,489,286; 5,466,239; 5,254,120 and 5,207,685. Other tympanostomy tubes are described in, e.g., U.S. Pat. Nos. 6,406,453; 5,178,623; 4,808,171 and 4,744,792.

In another aspect, the ear ventilation tube may be used to establish the normal function of the Eustachian tube and thus, attempt to resolve the stenosis that prevents its normal function. Fluid in the middle ear cavity normally secretes away from the tympanic membrane and thus, restoring the normal function of the Eustachian tube may provide optimal ventilation and drainage. For example, the ventilation tube may be an Eustachian stent composed of a hollow tubular body having a compressible core with two connected parallel arms and a radially-oriented flange, which is placed in the Eustachian tube to maintain patency. See, e.g., U.S. Pat. No. 6,589,286. The ventilation tube may be an Eustachian tube prosthesis composed of a flexible tube having a flange that extends radially for positioning within the Eustachian tube passageway. See, e.g., U.S. Pat. No. 4,015,607.

Tympanostomy tubes, which may be combined with drug combination (or individual component(s) thereof) according to the present invention, include commercially available products. For example, Medtronic Xomed, Inc. (Jackonsville, Fla.) sells a variety of ear ventilation tubes, including Long-Term Ventilation Tubes and Grommet Style Ventilation Tubes, including ARMSTRONG Grommets, GOODE T-Grommets, VENTURI Style Ventilation Tubes, SHEEHY Type Collar Buttons, REUTER Bobbins, COHEN T-Grommets, and SOILEAU TYTAN Titanium Tubes. Micromedics, Inc. (Eagan, Minn.) also sells a variety of ear ventilation tubes, including BAXTER Bevel Buttons, TINY TOUMA, SPOONER, TOUMA T-Tubes, SHOEHORN Bobbins, SHAH, and SILVERSTEIN MICROWICK Eustachian Tubes. Gyrus ENT LLC (Bartlett, Tenn.) also sells a variety of ear ventilation tubes, including ULTRASIL Ventilation Tubes, RICHARDS COLLAR Bobbins, BALDWIN BUTTERFLY Ventilation Tubes and PAPARELLA 2000 Tubes.

In one aspect, the present invention provides ear ventilation tube devices that include an anti-scarring drug combination (or individual component(s) thereof) or a composition that includes an anti-scarring drug combination (or individual component(s) thereof). Numerous polymeric and non-polymeric delivery systems for use in ear ventilation tubes have been described above. These compositions can further include a fibrosis-inhibiting drug combination (or individual component(s) thereof) such that the overgrowth of granulation tissue is inhibited or reduced.

Numerous polymeric and non-polymeric delivery systems for use in ear ventilation tubes have been described above. Methods for incorporating the fibrosis-inhibiting drug combination (or individual component(s) thereof) or a composition comprising the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) a coating applied to the external surface of the ear ventilation tube; (b) a coating applied to the internal (luminal) surface of the ear ventilation tube; or (c) a coating applied to all or parts of both surfaces. In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device. In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting drug combination (or individual component(s) thereof) described above can be utilized in the practice of this embodiment. Within one embodiment of the invention, ear ventilation tubes may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As ear ventilation tube devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations for use in ear ventilation tubes include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the ear ventilation tube device, the anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combination (or individual component(s) thereof) that can be used in conjunction with ear ventilation tube devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Intraocular Implants

In another aspect, the present invention provides for the combination of an anti-scarring drug combination (or individual component(s) thereof) and an intraocular implant.

In one embodiment, the intraocular implant is an intraocular lens device for the prevention of lens (e.g., anterior or posterior lens) opacification. Eyesight deficiencies that may be treated with intraocular lenses include, without limitation, cataracts, myopia, hyperopia, astigmatism and other eye diseases. Intraocular lenses are most commonly used to replace the natural crystalline lens which is removed during cataract surgery. A cataract results from a change in the transparency of the normal crystalline lens in the eye. When the lens becomes opaque from calcification (e.g., yellow and/or cloudy), the light cannot enter the eye properly and vision is impaired.

Implantation of intraocular lenses into the eye is a standard technique to restore useful vision in diseased or damaged eyes. The number of intraocular lenses implanted in the United States has grown exponentially over the last decade. Currently, over 1 million intraocular lenses are implanted annually, with the vast majority (90%) being placed in the posterior chamber of the eye. The intent of intraocular lenses is to replace the natural crystalline lens (i.e., aphakic eye) or to supplement and correct refractive errors (i.e., phakic eye, natural crystalline lens is not removed).

Implanted intraocular lenses may develop complications caused by mechanical trauma, inflammation, infection or optical problems. Mechanical and inflammatory injury may lead to reduced vision, chronic pain, secondary cataracts, corneal decompensation, cystoid macular edema, hyphema, uveitis or glaucoma. One common problem that occurs with cataract extraction is opacification which results from the tissue's reaction to the surgical procedure or to the artificial lens. Opacification leads to clouding of the intraocular lens, thus reducing the long-term benefits. Opacification typically results when proliferation and migration of epithelial cells occur along the posterior capsule behind the intraocular lens. Subsequent surgery may be required to correct this reaction; however, it involves a complex technical process and may lead to further serious, sight-threatening complications. Therefore, coating or incorporating the intraocular lens with a fibrosis-inhibiting agent may reduce these complications.

Representative examples of intraocular lenses that can benefit from being coated with or having incorporated therein a fibrosis-inhibiting agent include, without limitation, polymethylmethacrylate (PMMA) intraocular lenses, silicone intraocular lenses, achromatic lenses, pseudophakos, phakic lenses, aphakic lenses, multi-focal intraocular lenses, hydrophilic and hydrophobic acrylic intraocular lenses, intraocular implants, optic lenses and rigid gas permeable (RGP) lenses.

In one aspect, intraocular lenses may be foldable or rigid. The foldable lenses may be inserted in a small incision site using a tiny tube whereas the hard lenses are inserted through a larger incision site. Foldable lenses may be composed of silicone, acrylic or hydrogel whereas rigid lenses may be composed of hard polymeric compositions (PMMA).

In one aspect, the intraocular lens may be used as an implant for the treatment of cataracts, where the natural crystalline lens of the eye has been removed (i.e., aphakic lens). For example, the intraocular lens may be composed of two lenses having distinct refractive indices and distinct optical powers being joined together as an achromatic lens that may be connected within a posterior or anterior chamber of the eye. See, e.g., U.S. Pat. No. 5,201,762. The intraocular lens may be secured in the posterior chamber by a system of posts that protrude through the iris attached to retaining rings. See, e.g., U.S. Pat. No. 4,053,953. The intraocular lens may be hard with a shape memory which is capable of deforming for insertion into the eye but will harden at normal body temperature. See, e.g., U.S. Pat. No. 4,946,470. The intraocular lens may be coated with proteins, polypeptides, polyamino acids, polyamines or carbohydrates bound to the surface of the implant. See, e.g., U.S. Pat. Nos. 6,454,802 and 6,106,554. Other examples of aphakic intraocular lenses are described in, e.g., U.S. Pat. Nos. 6,599,317; 6,585,768; 6,558,419; 6,533,813; 6,210,438; 5,266,074; 4,753,654; 4,718,904 and 4,704,123.

In another aspect, the intraocular lens may be used as a corrective implant for vision impairment, where the natural crystalline lens of the eye has not been removed (i.e., phakic lens). For example, the intraocular lens may be a narrow profile, glare reducing, phakic anterior chamber lens that may be composed of an optic zone and a transition zone that has a curvature shaped to minimize direct glare. See, e.g., U.S. Pat. No. 6,596,025. The intraocular lens may be a self-centering phakic lens inserted in the posterior chamber lens in which arms (i.e., haptic bodies) extend outwardly and protrude into the pupil such that the iris provides centering force to keep lens in place. See, e.g., U.S. Pat. No. 6,015,435. The intraocular lens may be composed of a circumferential edge and two haptics extending from the edge to a transverse member which is substantially straight or bowed inward toward the lens. See, e.g., U.S. Pat. No. 6,241,777. Other examples of phakic intraocular lenses are described in, e.g., U.S. Pat. Nos. 6,228,115; 5,480,428 and 5,222,981.

In another aspect, the intraocular lens may be a multi-focal lens capable of variable accommodation to enable the user to look through different portions of the lens to achieve different levels of focusing power. For example, the intraocular lens may be a variable focus lens composed of two lens portions with an optical zone between the lenses which may contain a fluid reservoir and channel containing charged solution. See, e.g., U.S. Pat. No. 5,443,506.

In another aspect, intraocular lenses may be deformable such that the lens may be folded for insertion through a tunnel incision. For example, the intraocular lens may be composed of a lens with fixation members for retaining the lens in the eye which may be configured for folding or rolling from a normal optical condition into an insertion condition to permit the lens to be passed through an incision into the eye. See, e.g., U.S. Pat. No. 5,476,513. The intraocular lens may be composed of a resilient, deformable silicone based optic with a fixation means coupled to the optic for retaining the optic in the eye. See, e.g., U.S. Pat. No. 5,201,763. The intraocular lens may be composed of a copolymer of three constituents which may be deformable from its original shape. See, e.g., U.S. Pat. No. 5,359,021. The intraocular lens may be composed of a transparent, flexible membrane with an interior sac and an attached bladder, in which optical fluid medium is shunted from the optical element to the bladder to aid in its deformity during insertion. See, e.g., U.S. Pat. No. 6,048,364. The intraocular lens may be a biocomposite composed of an optic portion made of high water content hydrogel capable of being folded and a haptic portion of low water content hydrogel having strength and rigidity. See, e.g., U.S. Pat. No. 5,211,662. Other deformable intraocular lenses are described in, e.g., U.S. Pat. Nos. 6,267,784; 5,507,806 and U.S. Patent Application Publication No. 2003/0114928A1.

Other related devices and/or compositions (e.g., insertion devices) that may be used in conjunction with intraocular lenses are described in, e.g., U.S. Pat. Nos. 6,629,979; 6,187,042; 6,113,633; 4,740,282 and U.S. Patent Application Publication Nos. 2003/0212409A1 and 2003/0187455A1.

Intraocular lenses, which may be combined with drug combinations (or individual components thereof) according to the present invention, include commercially available products. For example, Alcon Laboratories, Inc. (Fort Worth, Tex.) sells the foldable ACRYSOF Intraocular Lens. Bausch & Lomb Surgical, Inc. (San Dimas, Calif.) sells the foldable SOFLEX SE Intraocular Lens. Advanced Medical Optics, Inc (Santa Ana, Calif.) sells the CLARIFLEX Foldable Intraocular Lens, SENSAR Acrylic Intraocular Lens, and PHACOFLEX II SI4ONB and SI3ONB.

The intraocular implant may comprise the fibrosis-inhibiting drug combination (or individual component(s) thereof) or a composition that includes the fibrosis-inhibiting drug combination (or individual component(s) thereof) directly. Alternatively, or in addition, the agent may be coated, absorbed into, or bound onto the lens surface (e.g., to the haptics), or may be released from a hole (pore) or cavity outside the optical part of the lens surface.

The intraocular implants of the invention may be used in various surgical procedures. For example, the intraocular implant may be used in conjunction with a transplant for the cornea. Synthetic corneas can be used in patients loosing vision due to a degenarative cornea. Implanted synthetic corneas can restore patient vision, however, they often induce a fibrous foreign body response that limits their use. The intraocular implant of the present invention can prevent the foreign body response to the synthetic cornea and extend the cornea longevity. In another example, the synthetic cornea itself is coated with the agents of the invention, thus minimizing tissue reaction to corneal implantation.

In another aspect, the intraocular lens may be used in conjunction with treatment of secondary cataract after extracapsular cataract extraction.

As described above, the present invention provides intraocular lenses and other implants that include an anti-scarring drug combination (or individual component(s) thereof) or a composition that includes an anti-scarring drug combination (or individual component(s) thereof). In one aspect, the anti-scarring drug combination may be selected from: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Numerous polymeric and non-polymeric delivery systems for use in intraocular implants have been described above.

Methods for coating fibrosis-inhibiting compositions onto or into the implants include: (a) directly affixing to the implants a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the implant a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier (c) by coating the implant with a substance such as a hydrogel which will in turn absorb a fibrosis-inhibiting drug combination (or individual component(s) thereof) or a composition comprising a fibrosis-inhibiting drug combination (or individual component(s) thereof), (d) constructing the implant itself or a portion of the lens with a fibrosis-inhibiting drug combination (or individual component(s) thereof) or a composition comprising a fibrosis-inhibiting drug combination (or individual component(s) thereof), or (e) by covalently binding a fibrosis-inhibiting drug combination (or individual component(s) thereof) or a composition comprising a fibrosis-inhibiting drug combination (or individual component(s) thereof) directly to the lens surface or to a linker (small molecule or polymer) that is coated or attached to the implant surface. For these devices, the coating process can be performed in such a manner as to (a) coat the posterior surface of the specific implant, (b) coat the anterior surface of the implant or (c) coat all or parts of both the posterior and anterior surfaces of the device. The protruding arms of the implant can also be coated with the fibrosis-inhibiting drug combination (or individual component(s) thereof).

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device.

The process of coating these implants with a fibrosis-inhibiting drug combination (or individual component(s) thereof) or incorporating the fibrosis-inhibiting drug combination (or individual component(s) thereof) into the implant and the materials selected for these processes are such that they do not significantly alter the refractive index of the intraocular implant or the visible light transmission of the implant or lens.

According to the present invention, any scarring drug combination (or individual component(s) thereof) described above can be utilized in the practice of this embodiment. Within one embodiment of the invention, intraocular implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As intraocular implants are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in intraocular implants include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the intraocular implant, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combination (or individual component(s) thereof) that can be used in conjunction with intraocular implants in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Hypertrophic Scars and Keloids

In another aspect, the present invention provides for the combination of an anti-scarring drug combination (or individual component(s) thereof) and a device for use in treating hypertrophic scars and keloids.

Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs.

A variety of devices for treating hypertrophic scars and keloids have been described. For example, the device may be an external tissue expansion device composed of two suture steel plates with adhesive attached foam cushions which apply constant continuous low grade force to skin and tissue to provide removal of hypertrophic scars and keloids. See, e.g., U.S. Pat. No. 6,254,624. The device may be a masking element which is pressed onto the scar tissue with an adjustable force by means of a pressure control unit and is connected with inflatable or suction members in the masking element. See, e.g., U.S. Pat. No. 6,013,094. The treatment may be a device having locking elements and grasping structures such that the dermal and epidermal layers of a skin wound can be pushed together such that the tissue edges are abutting, such that a wound may be closed with minimal scarring. See, e.g., U.S. Pat. No. 5,591,206.

In another aspect, the hypertrophic scar or keloid may be treated by using a device in conjunction with a coating or sheet that may be used to deliver either anti-scarring agents alone, or anti-scarring compositions as described above. For example, the coating or sheet may be a copolymer composed of a hydrophilic polymer, such as polyethylene glycol, that is bound to a polymer that adsorbs readily to the surfaces of body tissues, such as phenylboronic acid. See, e.g., U.S. Pat. No. 6,596,267. The coating or sheet may be a self-adhering silicone sheet which is impregnated with an antioxidant and/or antimicrobial. See, e.g., U.S. Pat. No. 6,572,878. The coating or sheet may be a wound dressing garment composed of an outer pliable layer and a self-adhesive inner gel lining which serves as a dressing for contacting wounds. See, e.g., U.S. Pat. No. 6,548,728. The coating or sheet may be a liquid composition composed of a film-forming carrier such as a collodion which contains one or more active ingredients such as a topical steroid, silicone gel and vitamin E. See, e.g., U.S. Pat. No. 6,337,076. The coating or sheet may be a bandage with a scar treatment pad with a layer of silicone elastomer or silicone gel. See, e.g., U.S. Pat. Nos. 6,284,941 and 5,891,076.

In another aspect, a medical device may be used in conjunction with an injectable composition that may be directly injected into a hypertrophic scar or keloid, in order to prevent the progression of these lesions. The frequency of injections will depend upon the release kinetics of the polymer used (if present), and the clinical response. This therapy is of particular value in the prophylactic treatment of conditions which are known to result in the development of hypertrophic scars and keloids (e.g., bums), and is preferably initiated after the proliferative phase has had time to progress (approximately 14 days after the initial injury), but before hypertrophic scar or keloid development. For example, an injectable treatment for hypertrophic scars and keloids may include the administration of an effective amount of angiogenesis inhibitor (e.g., fumagillol, thalidomide) as a systemic or local treatment to decrease excessive scarring. See, e.g., U.S. Pat. No. 6,638,949. The injectable treatment may be a cryoprobe containing cryogen whereby it is positioned within the hypertrophic scar or keloid to freeze the tissue. See, e.g., U.S. Pat. No. 6,503,246. The injectable treatment may be a method of locally administering an amount of botulinum toxin in or in close proximity to the skin wound, such that the healing is enhanced. See, e.g., U.S. Pat. No. 6,447,787. The injectable treatment may be a method of administering an antifibrotic amount of fluoroquinolone to prevent or treat scar tissue formation. See, e.g., U.S. Pat. No. 6,060,474. The injectable treatment may be a composition of an effective amount of calcium antagonist and protein synthesis inhibitor sufficient to cause matrix degradation at a scar site so as to control scar formation. See, e.g., U.S. Pat. No. 5,902,609. The injectable treatment may be a composition of non-biodegradable microspheres with a substantial surface charge in a pharmaceutically acceptable carrier. See, e.g., U.S. Pat. No. 5,861,149. The injectable treatment may be a composition of endothelial cell growth factor and heparin which may be administered topically or by intralesional injection. See, e.g., U.S. Pat. No. 5,500,409.

Treatments and devices used for hypertrophic scars and keloids, which may be combined with drug combinations (or individual components thereof) according to the present invention, include commercially available products. Representative products include, for example, PROXIDERM External Tissue Expansion product for wound healing from Progressive Surgical Products (Westbury, N.Y.), CICA-CARE Gel Sheet dressing product from Smith & Nephew Healthcare Ltd. (India), and MEPIFORM Self-Adherent Silicone Dressing from Molnlycke Health Care (Eddystone, Pa.).

In one aspect, devices for the treatment of hypertrophic scars and keloids may be combined with a topical or injectable composition that includes an anti-scarring drug combination (or individual component(s) thereof) and a polymeric carrier suitable for application on or into hypertrophic scars or keloids. Incorporation of a fibrosis-inhibiting drug combination (or individual component(s) thereof) into a topical formulation or an injectable formulation is one approach to treat this condition. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are described above.

In another embodiment, the therapeutic agent can be incorporated into a secondary carrier (e.g., micelles, liposomes, emulsions, microspheres, nanospheres etc, as described above). Microsphere and nanospheres may include degradable polymers. Degradable polymers that can be used include poly(hydroxyl esters) (e.g., PLGA, PLA, PCL, and the like) as well as polyanhydrides, polyorthoesters and polysaccharides (e.g., chitosan and alginates).

Within another aspect of the invention, these fibrosis-inhibiting drug combination (or individual component(s) thereof)/secondary carrier compositions can be a) incorporated directly into or onto the device, b) incorporated into a solution, c) incorporated into a gel or viscous solution, d) incorporated into the composition used for coating the device or e) incorporated into or onto the device following coating of the device with a coating composition. For example, fibrosis-inhibiting drug combination (or individual component(s) thereof) loaded PLGA microspheres can be incorporated into a polyurethane coating solution which is then coated onto the device.

According to the present invention, any fibrosis-inhibiting drug combination (or individual component(s) thereof) described above can be utilized in the practice of this embodiment. Within one embodiment of the invention, devices for the treatment of hypertrophic scars and keloids may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As devices for preventing hypertrophic scarring or keloids are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use devices for treating hypertrophic scars and keloids include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the device, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with devices for treating hypertrophic scars and keloids in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Vascular Grafts

In one aspect, the present invention provides for the combination of an anti-scarring drug combination (or individual component(s) thereof) and a vascular graft. Vascular graft devices that include a fibrosis-inhibiting drug combination (or individual component(s) thereof) are capable of inhibiting or reducing the overgrowth of granulation tissue, which can improve the clinical efficacy of these devices.

The vascular graft may be an extravascular graft or an intravascular (i.e., endoluminal) graft. The vascular graft may be, without limitation, in the form of a peripheral bypass application or a coronary bypass application. Vascular grafts may be used to replace or substitute damaged or diseased veins and arteries, including, without limitation, blood vessels damaged by aneurysms, intimal hyperplasia and thrombosis. Vascular grafts may also be used to provide access to blood vessels, for example, for hemodialysis access. Vascular grafts are implanted, for example, to provide an alternative conduit for blood flow through damaged or diseased areas in veins and arteries, including, without limitation, blood vessels damaged by aneurysms, intimal hyperplasia and thrombosis, however, the graft may lead to further complications, including, without limitation, infections, inflammation, thrombosis and intimal hyperplasia. The lack of long-term patency with vascular grafts may be due, for example, to surgical injury and abnormal hemodynamics and material mismatch at the suture line. Typically, further disease (e.g., restenosis) of the vessel occurs along the bed of the artery.

Some forms of improvements to vascular grafts have been made in an attempt to reduce the restenosis that occurs at the anastomosis site. Improvements include: (a) using a Miller cuff, which is a small piece of natural vein to make a short cuff that is joined by stitching it to the artery opening and the prosthetic graft; (b) using a flanged graft whereby the graft has a terminal skirt or cuff that facilitates an end-to-side anastomosis; (c) using a graft with an enlarged chamber having a large diameter for suture at the anastomosis site; and (d) using a graft that dispensing an agent that prevents thrombosis and/or intimal hyperplasia.

Representative examples of vascular grafts include, without limitation, synthetic bypass grafts (e.g., femoral-popliteal, femoral-femoral, axillary-femoral, and the like), vein grafts (e.g., peripheral and coronary), and internal mammary (e.g., coronary) grafts, bifurcated vascular grafts, intraluminal grafts, endovascular grafts and prosthetic grafts. Synthetic grafts can be made from a variety of polymeric materials, such as, for example, polytetrafluoroethylene (e.g., ePTFE), polyesters such as DACRON, polyurethanes, and combinations of polymeric materials.

Endoluminal vascular grafts may be used to treat aneurysms. For example, the vascular graft may be composed of a tubular graft with two tubular self-expanding stents that may be implanted for the treatment of aneurysms by means of minimally invasive procedures. See, e.g., U.S. Pat. No. 6,168,620. The vascular graft may be composed of a flexible tubular body and a compressible frame positioned against the tubular body for support which has pores on the surface to promote ingrowth. See, e.g., U.S. Pat. No. 5,693,088. The vascular graft may be bifurcated endovascular graft having a tubular trunk and two tubular limbs. See, e.g., U.S. Pat. No. 6,454,796. The vascular graft may be a kink-resistant endoluminal bifurcated graft having two separate lumens contacted by a single lumen section. See, e.g., U.S. Pat. No. 6,551,350. The vascular graft may be an intraluminal tube composed of ePTFE that has a seamline formed by overlapping the edges such that the microstructure fibrils are oriented in perpendicular directions. See, e.g., U.S. Pat. No. 5,718,973.

In another aspect, the vascular graft may be used as a conduit to bypass vascular stenosis or other vascular abnormalities. For example, the vascular graft may be composed of a porous material having a layer of porous hollow fibers positioned along the inner surface which allows for tissue growth while inhibiting bleeding during the healing process. See, e.g., U.S. Pat. No. 5,024,671. The vascular graft may be a flexible, monolithic, reinforced polymer tube having a microporous ePTFE tubular member and external ePTFE rib members projecting outwardly from the outer wall. See, e.g., U.S. Pat. No. 5,609,624. The vascular graft may be composed of a tubular wall having longitudinally extending pleats that respond flexurally to changes in blood pressure while maintaining high compliance with reduced kinking. See, e.g., U.S. Pat. No. 5,653,745. The vascular graft may be a radially supported ePTFE tube that is reinforced with greater density ring-shaped regions. See, e.g., U.S. Pat. No. 5,747,128. The vascular graft may be porous PTFE tubing composed of a microstructure of nodes interconnected by fibrils which has a coating of elastomer on the outer wall. See, e.g., U.S. Pat. Nos. 5,152,782 and 4,955,899. The vascular graft may be a plurality of polymeric fibers knitted together composed of at least three different fibers in which two fibers are absorbable and one is non-absorbable. See, e.g., U.S. Pat. Nos. 4,997,440; 4,871,365 and 4,652,264.

In another aspect, the vascular graft may be modified to reduce thrombus formation or intimal hyperplasia at the anastomotic site. For example, the vascular graft may have an enlarged chamber having a first diameter parallel to the axis of the tubular wall and a second diameter transverse to the axis of the tube. See, e.g., U.S. Pat. No. 6,589,278. The vascular graft may have a flanged skirt or cuff section with facilitates an end-to-side anastomosis directly between the artery and the end of the flanged bypass graft. See, e.g., U.S. Pat. No. 6,273,912. The vascular graft may be composed of a tubular wall having a non-thrombogenic agent within the luminal layer and a thrombogenic layer forming the exterior of the vascular graft. See, e.g., U.S. Pat. No. 6,440,166. The vascular graft may be composed of a smooth luminal surface made of ePTFE with a small pore size to reduce adherence of occlusive blood components. See, e.g., U.S. Pat. No. 6,517,571. The vascular graft may be composed of hollow tubing that contains drug that is helically wrapped around the outer wall of a porous ePTFE graft whereby drug is dispensed by infusion through the porous interstices of the graft wall. See, e.g., U.S. Pat. No. 6,355,063.

In another aspect, the vascular graft may be a harvested blood vessel that is used for bypass grafting. For example, vascular grafts may be composed of harvested arterial vessels from a host, such as the internal mammary arteries or inferior epigastric arteries. See, e.g., U.S. Pat. No. 5,797,946. Vascular grafts may also be composed of saphenous veins which may be harvested from the host and used for coronary bypass or peripheral bypass procedures. See, e.g., U.S. Pat. No. 6,558,313.

Other examples of vascular grafts are described in U.S. Pat. Nos. 3,096,560, 3,805,301, 3,945,052, 4,140,126, 4,323,525, 4,355,426, 4,475,972, 4,530,113, 4,550,447, 4,562,596, 4,601,718, 4,647,416, 4,878,908, 5,024,671, 5,104,399, 5,116,360, 5,151,105, 5,197,977, 5,282,824, 5,405,379, 5,609,624, 5,693,088, and 5,910,168.

Vascular grafts, which may be combined with one or more agents according to the present invention, include commercially available products. GORE-TEX Vascular Grafts and GORE-TEX INTERING Vascular Grafts are sold by Gore Medical Division (W. L. Gore & Associates, Inc. Newark, Del.). C.R. Bard, Inc. (Murray Hill, N.J.) sells the DISTAFLO Bypass Grafts and IMPRA CARBOFLO Vascular Grafts.

In one aspect, the anti-scarring drug combination (or individual component(s) thereof) or a composition containing the anti-scarring drug combination (or individual component(s) thereof) is combined with a vascular graft.

Numerous polymeric and non-polymeric delivery systems for use in vascular grafts have been described above. Methods for incorporating fibrosis-inhibiting drug combinations (or individual components thereof) or compositions that comprising fibrosis-inhibiting drug combinations (or individual components thereof) onto or into the graft include: (a) affixing (directly or indirectly) to the graft a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) incorporating or impregnating into the graft a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the graft with a substance such as a hydrogel which will in turn absorb a fibrosis-inhibiting drug combination (or individual component(s) thereof) or a composition comprising a fibrosis-inhibiting drug combination (or individual component(s) thereof), (d) constructing the graft itself or a portion of the graft with a fibrosis-inhibiting drug combination (or individual component(s) thereof) or a composition comprising a fibrosis-inhibiting drug combination (or individual component(s) thereof), or (e) by covalently binding a fibrosis-inhibiting drug combination (or individual component(s) thereof) or a composition comprising a fibrosis-inhibiting drug combination (or individual component(s) thereof) directly to the graft surface or to a linker (small molecule or polymer) that is coated or attached to the graft surface. For these grafts, the coating process can be performed in such a manner as to (a) coat the external surface of the graft, (b) coat the interior (luminal) surface of the graft, or (c) coat all or parts of both the external and internal surfaces of the graft, or (d) coat at least one end of the graft.

The fibrosis-inhibiting drug combination (or individual component(s) thereof) can be incorporated directly into the coating composition or into a secondary carrier (e.g., micelles, liposomes, emulsions, microspheres, nanospheres etc, as described above). Microsphere and nanospheres may include degradable polymers. Degradable polymers that can be used include poly(hydroxyl esters) (e.g., PLGA, PLA, PCL, and the like) as well as polyanhydrides, polyorthoesters and polysaccharides (e.g., chitosan and alginates).

In yet another embodiment, a gel, paste, thermogel or in situ forming gel that includes a fibrosis-inhibiting drug combination (or individual component(s) thereof) can be applied in a perivascular manner to the anastomosis produced during implantation of the graft device. Numerous polymeric and non-polymeric delivery systems for use in paste and gel formulations have been described above. The fibrosis-inhibiting drug combination (or individual component(s) thereof) can be incorporated directly into the gel or paste composition, or the therapeutic agent can be incorporated into a secondary carrier (e.g., micelles, liposomes, emulsions, microspheres, nanospheres etc, as described above).

In another aspect, the fibrosis-inhibiting drug combination (or individual component(s) thereof) can be incorporated into or onto an implant (e.g., a film or mesh material), which can be used in conjunction with a vascular graft to inhibit scarring at an anastomotic site. For example, a film or mesh material may be placed or wrapped in a perivascular (periadventitial) manner around the outside of the anastomosis at the time of surgery. Film and mesh implants may be used with a various types of vascular grafts, including synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts). Representative examples of films and meshes are described in further detail below.

In addition to the fibrosis-inhibiting drug combination (or individual component(s) thereof), the vascular graft devices compositions for use with vascular graft devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin). The combination of agents may be coated onto the entire or portions of the vascular graft such that the thrombogenicity and/or fibrosis is reduced or inhibited. In certain embodiments, these agents may be coated onto the vascular graft using biodegradable polymers. For example, polymeric material that forms a gel in the pores and/or on the surface of the graft may be used, such as alginates, chitosan and chitosan sulfate, hyaluronic acid, dextran sulfate, PLURONIC polymers, chain extended PLURONIC polymers, polyester-polyether block copolymers of the various configurations (e.g., MePEG-PLA, PLA-PEG-PLA, and the like).

In one aspect, synthetic vascular grafts are provided that comprise, in addition to the anti-fibrosing drug combination (or individual component(s) thereof), a composition in the form of a biodegradable gel. The gel composition can have anti-thrombogenic properties or include an agent having anti-thrombogenic properties, which may or may not be released from the gel composition. Gel coated grafts may reduce or prevent early thrombotic events commonly associated with implantation of synthetic grafts.

Polymeric biodegradable gels may comprise, for example, a chain extended PLURONIC polymer. Chain extended polymers may include a PLURONIC polymer (e.g., F127, F87, or the like) that has been reacted with a difunctional molecule such as succinyl chloride to increase the molecular weight of the polymer and thereby increase the viscosity of the PLURONIC polymer. Chain extended polymers can be dissolved in a solvent and then coated onto the synthetic vascular graft.

Gel compositions may be formed from a combination of small and/or polymeric molecules having two or more electrophilic groups and two or more nucleophilic groups. For example, the formulations may include a combination of a multi-armed PEG molecule in which the terminal hydroxyl groups are activated with succinimidyl moieties and a multi-armed PEG molecule having terminal amino and/or sulfhydryl groups. The multi-armed PEG reagents may be dissolved separately in an appropriate solvent (e.g., aqueous buffer, IPA, dichloromethane, or a combination of solvents) and then sprayed sequentially or simultaneously onto the desired surface of the graft, such that the two components react to produce a crosslinked gel. The solvent may then be removed by air or vacuum drying.

In another embodiment, the composition may be formed from a polymer having two or more succinimidyl groups and a small molecule having two or more amino or sulfhydryl groups (e.g., dilysine). Alternatively, the polymer components can include two or more sulfhydryl groups or amino groups, and the small molecule contains two or more succinimidyl groups.

In yet another embodiment, gel coatings may be produced from polyester-polyether block copolymers of various configurations (e.g., X-Y, X-Y-X or Y-X-Y, R-(Y-X)ₙ, R-(X-Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.), R is a multifunctional initiator and copolymers as well as blends and copolymers thereof.) may be used to form the gel coating.

In one embodiment, the synthetic vascular graft is formed of a porous synthetic material such as expanded PTFE (ePTFE). A coating comprising a gel composition, such as described above, may be applied onto the entire graft or a portion of the graft surface (e.g., the interior surface of the graft or the ends of the graft). Further, the pores of the graft may be either partially or fully filled with the coating composition. The extent to which the coating occupies the pores of the device can be altered by changing the solvent used to dissolve the polymer. For example, a surface coating may be achieved by using a hydrophilic solvent such as water which will not wet the hydrophobic surface of an ePTFE graft. Coating from a solvent such as dichloromethane, which wets an ePTFE surface, can be used to coat the polymer composition onto the inner pore structure of the graft.

The gel formulations may have anti-thrombogenic properties due to the hydrophilicity. Hydrophilic coatings may be physically removed from the surface of the graft over time which may reduce the adhesion of platelets to the graft surface. Additionally, an anti-thrombogenic agent (e.g., heparin, fragments of heparin, organic soluble salts of heparin, sulfonated carbohydrates, warfarin, coumadin, coumarin, heparinoid, danaparoid, argatroban chitosan sulfate, or chondroitin sulfate) may be included into the formulation. In one embodiment, the anti-thrombotic agent(s) may be incorporated into microspheres. Other additives which may be added into gel compositions for use with vascular grafts include buffers, osmolality modifiers, viscosity modifiers, and hydrating agents (e.g., PEG, MePEG, and various sugars).

According to the present invention, any anti-scarring drug combination (or individual component(s) thereof) described above can be utilized in the practice of this embodiment. Within one embodiment of the invention, vascular grafts may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As vascular grafts are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use with vascular grafts include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the vascular graft, the exemplary anti-fibrosing drug combinations (or individual components thereof), used alone or in combination, should be administered under the following dosing guidelines. The total amount (dose) of anti-scarring agent(s)in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with vascular graft devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Hemodialysis Access Devices

In one aspect, the present invention provides for the combination of an anti-scarring drug combination (or individual component(s) thereof) and a hemodialysis access device. Hemodialysis dialysis access devices that include a fibrosis-inhibiting drug combination (or individual component(s) thereof) are capable of inhibiting or reducing the overgrowth of granulation tissue, which can improve the clinical efficacy of these devices.

Hemodialysis access devices may be used when blood needs to be removed, cleansed and then returned to the body. Hemodialysis regulates the body's fluid and chemical balances as well as removes waste from the blood stream that cannot be cleansed by a normally functioning kidney due to disease or injury. For hemodialysis to occur, the blood may be obtained through a hemodialysis access or vascular access, in which minor surgery is performed to provide access through an AV fistula or AV access graft. These hemodialysis access devices may develop complications, including infections, inflammation, thrombosis and intimal hyperplasia of the associated blood vessels. The lack of long-term patency with hemodialysis access may be due to surgical injury, abnormal hemodynamics and material mismatch at the suture line. Typically, further disease (e.g., restenosis) of the vessel occurs along the bed of the artery and/or at the site of anastomosis.

In addition to the AV fistulas and AV access grafts described above, implantable subcutaneous hemodialysis access systems such as the commercially available catheters, ports, and shunts, may also be used for hemodialysis patients. These access systems may consist of a small metallic or polymeric device or devices implanted underneath the skin. These devices may be connected to flexible tubes, which are inserted into a vessel to allow for blood access.

Representative examples of hemodialysis access devices include, without limitation, AV access grafts, venous catheters, vascular grafts, implantable ports, and AV shunts. Synthetic hemodialysis access devices can be made from metals or polymers, such as polytetrafluoroethylene (e.g., ePTFE), polyesters such as DACRON, polyurethanes, or combinations of these materials.

In one aspect, the hemodialysis access device may be an AV access graft. For example, the AV access graft may be composed of an implantable self-expanding flexible percutaneous stent-graft of open weave construction with ends being compressible and having an elastic layer arranged along a portion of its length. See, e.g., U.S. Pat. Nos. 5,755,775 and 5,591,226. The AV access graft may be composed of a tubular section with a generally constant diameter which tapers towards the venous end. See, e.g., U.S. Pat. No. 6,585,762. The AV access graft may be composed of a two microporous ePTFE tubes that are circumferentially disposed over each other with a polymeric layer interposed between such that the graft is self-sealing and exhibits superior radial tensile strength and suture hole elongation resistance. See, e.g., U.S. Pat. No. 6,428,571. The AV access graft may be composed of a coaxial double lumen tube with an inner and outer tube having a self-sealing, nonbiodegradable, polymeric adhesive between the tubes. See, e.g., U.S. Pat. No. 4,619,641. The AV access graft may be composed of a synthetic fabric having a high external velour profile which is woven or knitted to form a tubular prosthesis which has elastic fibers that allows self-sealing following a punctured state. See, e.g., U.S. Pat. No. 6,547,820. The AV access graft may be of tubular form having a base tube with the ablumenal surface covered with a deflectable material, such as a porous film, which is arranged adjacently to allow movement. See, e.g., U.S. Pat. No. 5,910,168.

In another aspect, the hemodialysis access device may be a catheter system. For example, the catheter system may be composed of a suction and return line that are adapted for disposition in the vascular system of the body and are connected to a subcutaneous connector port. See, e.g., U.S. Pat. Nos. 6,620,118 and 5,989,206. The catheter system may be an apparatus that is used to arterialize a vein by creating an AV fistula by inserting a catheter into a vein and a catheter into an adjacent artery. See, e.g., U.S. Pat. No. 6,464,665. The catheter system may be composed of a hollow sheath that provides percutaneous introduction of fistula-generating vascular catheters through a perforation in a vessel wall, such that the catheters generate an intervascular fistula on-demand between adjacent vessels. See, e.g., U.S. Pat. Nos. 6,099,542 and 5,830,224.

In another aspect, the hemodialysis access device may be used for an AV fistula. For example, the hemodialysis access device may be an AV fistula assembly composed of a synthetic coiled stent graft with helically-extending turns with gaps used to enhance the function of an AV fistula. See, e.g., U.S. Pat. No. 6,585,760.

In another aspect, the hemodialysis access device may be an implantable access port, shunt or valve. These devices may be implanted subcutaneously with communication to the blood supply and accessed using a percutaneous puncture. For example, the hemodialysis access device may be composed of housing having an entry port and an exit port to a passageway which has an elastomeric sealing valve that provides access into the exit port for a needle. See, e.g., U.S. Pat. No. 5,741,228. The hemodialysis access device may be a shunt composed of a slideable valve and flexible lid that has a fluid communication tube between the arterial and venous ends. See, e.g., U.S. Pat. No. 5,879,320. The hemodialysis access device may be a shunt in the form of a junction that has a connector with two legs that are inserted into the native blood vessel and one leg that is adapted for sealing to another blood vessel without punctures. See, e.g., U.S. Pat. No. 6,019,788. The hemodialysis access device may be a surface access double hemostatic valve that may be mounted on the wall of an AV graft for hemodialysis access. See, e.g., U.S. Pat. Nos. 6,004,301 and 6,090,067.

Hemodialysis access devices, which may be combined with a drug combination (or individual component(s) thereof)according to the present invention, include commercially available products. For example, hemodialysis access devices include products, such as the LIFESITE (Vasca Inc., Tewksbury, Mass.) and the DIALOCK catheters from Biolink Corp. (Middleboro, Mass.), VECTRA Vascular Access Grafts and VENAFLO Vascular Grafts from C.R. Bard, Inc. (Murray Hill, N.J.), and GORE-TEX Vascular Grafts and Stretch Vascular Grafts from Gore Medical Division (W. L. Gore & Associates, Inc. Newark, Del.).

In one aspect, the anti-scarring drug combination (or individual component(s) thereof) or a composition containing the anti-scarring drug combination (or individual component(s) thereof) is combined with a hemodialysis access device. Numerous polymeric and non-polymeric delivery systems for use in hemodialysis access devices have been described above. Methods for incorporating a fibrosis-inhibiting drug combination (or individual component(s) thereof) or a composition comprising a fibrosis-inhibiting drug combination (or individual component(s) thereof) onto or into the hemodialysis access device include: (a) directly affixing to the hemodialysis access device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the hemodialysis access device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the hemodialysis access device with a substance such as a hydrogel which will in turn absorb a fibrosis-inhibiting drug combination (or individual component(s) thereof) or a composition comprising a fibrosis-inhibiting drug combination (or individual component(s) thereof), (d) constructing the hemodialysis access device itself or a portion of the graft with a fibrosis-inhibiting drug combination (or individual component(s) thereof) or a composition comprising a fibrosis-inhibiting drug combination (or individual component(s) thereof), or (e) by covalently binding a fibrosis-inhibiting drug combination (or individual component(s) thereof) or a composition comprising a fibrosis-inhibiting drug combination (or individual component(s) thereof) directly to the hemodialysis access device surface or to a linker (small molecule or polymer) that is coated or attached to the hemodialysis access device surface. For devices that are coated, the coating process can be performed in such a manner as to (a) coat only the external surface of the device; (b) coat the internal (luminal) surface of the device; or (c) coat all or parts of both the external and internal surfaces.

In another aspect, the fibrosis-inhibiting drug combination (or individual component(s) thereof) or a composition containing the fibrosis-inhibiting drug combination (or individual component(s) thereof) can be incorporated into an implant, such as a film or mesh, which can be used in conjunction with a hemodialysis access device to inhibit scarring at the site of an anastomosis or fistula. These films or meshes may be placed or wrapped in a perivascular (periadventitial) manner around the outside of the fistula or anastomosis at the time of surgery. Representative examples of implants (i.e., meshes and films) for use with hemodialysis access devices are described below.

In yet another aspect, a composition in the form of, for example, a gel, paste, thermogel, or in situ forming gel, which includes a fibrosis-inhibiting drug combination (or individual component(s) thereof) can be applied in a perivascular manner to the fistula or anastomosis produced during implantation of the hemodialysis access device.

The fibrosis-inhibiting drug combination (or individual component(s) thereof) can be incorporated directly into the gel or paste composition, or the therapeutic agent can be incorporated into a secondary carrier (e.g., micelles, liposomes, emulsions, microspheres, nanospheres etc, as described above) that is then incorporated into the composition that is to be delivered. Microsphere and nanospheres may include degradable polymers. Degradable polymers that can be used include poly (hydroxyl esters) (e.g., PLGA, PLA, PCL, and the like) as well as polyanhydrides, polyorthoesters and polysaccharides (e.g., chitosan and alginates).

In addition to the fibrosis-inhibiting drug combination (or individual component(s) thereof), hemodialysis access devices and compositions for use with hemodialysis access devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin).

According to the present invention, any anti-scarring drug combination (or individual component(s) thereof) described above can be utilized in the practice of this embodiment. Within one embodiment of the invention, hemodialysis access devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Several examples of fibrosis-inhibiting drug combinations (or individual components thereof) for use with hemodialysis access devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

As hemodialysis access devices are made in a variety of configurations and sizes, the exact dose administered will vary with device size, surface area and design. However, certain principles can be applied in the application of this art. Drug dose can be calculated as a function of dose per unit area (of the portion of the device being coated), and total amount of drug on the device can be measured and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Regardless of the method of application of the drug to the device, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combination (or individual component(s) thereof) that can be used in conjunction with hemodialysis access devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Films and Meshes

In one aspect, the present invention provides for the combination of an anti-scarring drug combination (or individual component(s) thereof) and a film or mesh. Incorporation of a fibrosis-inhibiting drug combination (or individual component(s) thereof) into or onto a film or mesh can minimize fibrosis (or scarring) in the vicinity of the implant and may reduce or prevent the formation of adhesions between the implant and the surrounding tissue. In certain aspects, the film or mesh may be used as a drug-delivery vehicle (e.g., as a perivascular delivery device for the prevention of neointimal hyperplasia at an anastomotic site).

Films or meshes may take a variety of forms including, but not limited to, surgical barriers, surgical adhesion barriers, membranes (e.g., barrier membranes), surgical sheets, surgical patches (e.g., dural patches), surgical wraps (e.g., vascular, perivascular, adventitial, periadventitital wraps, and adventitial sheets), meshes (e.g., perivascular meshes), bandages, liquid bandages, surgical dressings, gauze, fabrics, tapes, surgical membranes, polymer matrices, shells, envelopes, tissue coverings, and other types of surgical matrices, scaffolds, and coatings.

In one aspect, the device comprises or may be in the form of a film. The film may be formed into one of many geometric shapes. Depending on the application, the film may be formed into the shape of a tube or may be a thin, elastic sheet of polymer. Generally, films are less than 5, 4, 3, 2, or 1 mm thick, more preferably less than 0.75 mm, 0.5 mm, 0.25 mm, or, 0.10 mm thick. Films can also be generated of thicknesses less than 50 μm, 25 μm or 10 μm. Films generally are flexible with a good tensile strength (e.g., greater than 50, preferably greater than 100, and more preferably greater than 150 or 200 N/cm²), good adhesive properties (i.e., adheres to moist or wet surfaces), and have controlled permeability. Polymeric films (which may be porous or non-porous) are particularly useful for application to the surface of a device or implant as well as to the surface of tissue, cavity or an organ.

Films may be made by several processes, including for example, by casting, and by spraying, or may be formed at the treatment site in situ. For example, a sprayable formulation may be applied onto the treatment site which then forms into a solid film.

In another aspect, the device may comprise or be in the form of a polymer, wherein at least some of the polymer is in the form of a mesh. A mesh, as used herein, is a material composed of a plurality of fibers or filaments (i.e., a fibrous material), where the fibers or filaments are arranged in such a manner (e.g., interwoven, knotted, braided, overlapping, looped, knitted, interlaced, intertwined, webbed, felted, and the like) so as to form a porous structure. Typically, a mesh is a pliable material, such that it has sufficient flexibility to be wrapped around the external surface of a body passageway or cavity, or a portion thereof. The mesh may be capable of providing support to the structure (e.g., the vessel or cavity wall) and may be adapted to release an amount of the therapeutic agent.

Mesh materials may take a variety of forms. For example, the mesh may be in a woven, knit, or non-woven form and may include fibers or filaments that are randomly oriented relative to each other or that are arranged in an ordered array or pattern. In one embodiment, for example, a mesh may be in the form of a fabric, such as, for example, a knitted, braided, crocheted, woven, non-woven (e.g., a melt-blown or wet-laid) or webbed fabric. In one embodiment, a mesh may include a natural or synthetic biodegradable polymer that may be formed into a knit mesh, a weave mesh, a sprayed mesh, a web mesh, a braided mesh, a looped mesh, and the like. Preferably, a mesh or wrap has intertwined threads that form a porous structure, which may be, for example, knitted, woven, or webbed.

The structure and properties of the mesh used in a device depend on the application and the desired mechanical (i.e., flexibility, tensile strength, and elasticity), degradation properties, and the desired loading and release characteristics for the selected therapeutic agent(s). The mesh should have mechanical properties, such that the device will remain sufficiently strong until the surrounding tissue has healed. Factors that affect the flexibility and mechanical strength of the mesh include, for example, the porosity, fabric thickness, fiber diameter, polymer composition (e.g., type of monomers and initiators), process conditions, and the additives that are used to prepare the material.

Typically, the mesh possesses sufficient porosity to permit the flow of fluids through the pores of the fiber network and to facilitate tissue ingrowth. Generally, the interstices of the mesh should be sufficiently wide apart to allow light visible by eye, or fluids, to pass through the pores. However, materials having a more compact structure also may be used. The flow of fluid through the interstices of the mesh depends on a variety of factors, including, for example, the stitch count or thread density. The porosity of the mesh may be further tailored by, for example, filling the interstices of the mesh with another material (e.g., particles or polymer) or by processing the mesh (e.g., by heating) in order to reduce the pore size and to create non-fibrous areas. Fluid flow through the mesh of the invention will vary depending on the properties of the fluid, such as viscosity, hydrophilicity/hydrophobicity, ionic concentration, temperature, elasticity, pseudoplasticity, particulate content, and the like. Preferably, the interstices do not prevent the release of impregnated or coated therapeutic agent(s) from the mesh, and the interstices preferably do not prevent the exchange of tissue fluid at the application site.

Mesh materials should be sufficiently flexible so as to be capable of being wrapped around all or a portion of the external surface of a body passageway or cavity. Flexible mesh materials are typically in the form of flexible woven or knitted sheets having a thickness ranging from about 25 microns to about 3000 microns; preferably from about 50 to about 1000 microns. Mesh material suitable for wrapping around arteries and veins typically ranges from about 100 to 400 microns in thickness.

The diameter and length of the fibers or filaments may range in size depending on the form of the material (e.g., knit, woven, or non-woven), and the desired elasticity, porosity, surface area, flexibility, and tensile strength. The fibers may be of any length, ranging from short filaments to long threads (i.e., several microns to hundreds of meters in length). Depending on the application, the fibers may have a monofilament or a multifilament construction.

The mesh may include fibers that are of same dimension or of different dimensions, and the fibers may be formed from the same or different types of biodegradable polymers. Woven materials, for example, may include a regular or irregular array of warp and weft strands and may include one type of polymer in the weft direction and another type (having the same or a different degradation profile from the first polymer) in the warp direction. The degradation profile of the weft polymer may be different than or the same as the degradation profile of the warp polymer. Similarly, knit materials may include one or more types (e.g., monofilament, multi-filament) and sizes of fibers and may include fibers made from the same or from different types of biodegradable polymers.

The structure of the mesh (e.g., fiber density and porosity) may impact the amount of therapeutic agent that may be loaded into or onto the device. For example, a fabric having a loose weave characterized by a low fiber density and high porosity will have a lower thread count, resulting in a reduced total fiber volume and surface area. As a result, the amount of agent that may be loaded into or onto, with a fixed carrier: therapeutic agent ratio, the fibers will be lower than for a fabric having a high fiber density and lower porosity. It is preferable that the mesh also should not invoke biologically detrimental inflammatory or toxic response, should be capable of being fully metabolized in the body, have an acceptable shelf life, and be easily sterilized.

The device may include multiple mesh materials in any combination or arrangement. For example, a portion of the device may be a knitted material and another portion may be a woven material. In another embodiment, the device may more than one layer (e.g., a layer of woven material fused to a layer of knitted material or to another layer of the same type or a different type of woven material). In some embodiments, multi-layer constructions (e.g., device having two or more layers of material) may be used, for example, to enhance the performance properties of the device (e.g., for enhancing the rigidity or for altering the porosity, elasticity, or tensile strength of the device) or for increasing the amount of drug loading.

Multi-layer constructions may be useful, for example, in devices containing more than one type of therapeutic agent. For example, a first layer of mesh material may be loaded with one type of agent and a second layer may be loaded with another type of agent. The two layers may be unconnected or connected (e.g., fused together, such as by heat welding or ultrasonic welding) and may be formed of the same type of fabric or from a different type of fabric having a different polymer composition and/or structure.

In certain aspects, a mesh may include portions that are not in the form of a mesh. For example, the device may include the form of a film, sheet, paste, and the like, and combinations thereof. For example, the device may have a multi-layer construction having a film layer that includes the therapeutic agent and one or more layers of mesh material. For example, the film layer may be interposed between two layers of mesh or may be disposed on just one side the mesh material. The film layer may include a first therapeutic agent, whereas one or more of the layers of mesh may include the same or a different agent. In another embodiment, the device includes at least two layers of mesh. In one aspect, at least two of the at least two layers of mesh are fused together.

In one aspect, multilayer devices are provided that may further include a film layer. The film layer may reside between two of the at least two layers of mesh. In yet another embodiment, a delivery device is described that includes a mesh, wherein the mesh includes a biodegradable polymer and a first therapeutic agent. The device may further include a film that includes a second therapeutic agent, which may have the same or a different composition than the first therapeutic agent. For example, in one embodiment, a device suitable for wrapping around a vein or artery includes a layer of mesh and a film layer loaded with a therapeutic agent. The device may be wrapped around a body passageway or cavity, such that the film layer contacts the external surface of the passageway or cavity. Thus, the device may deliver the appropriate dosage of agent and may provide sufficient mechanical strength to improve and maintain the structural integrity of the body passageway or cavity.

In one aspect, the mesh or film includes a polymer. The polymer may be a biodegradable polymer. Biodegradable compositions that may be used to prepare the mesh include polymers that comprise albumin, collagen, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and ε-caprolactone, copolymers of lactide and 1,4-dioxane-2-one, polymers and copolymers that include one or more of the residue units of the monomers D-lactide, L-lactide, D,L-lactide, glycolide, ε-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids). These compositions include copolymers of the above polymers as well as blends and combinations of the above polymers. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

In one aspect, the mesh or film includes a biodegradable or resorbable polymer that is formed from one or more monomers selected from the group consisting of lactide, glycolide, e-caprolactone, trimethylene carbonate, 1,4-dioxan-2-one, 1,5-dioxepan-2-one, 1,4-dioxepan-2-one, hydroxyvalerate, and hydroxybutyrate. In one aspect, the polymer may include, for example, a copolymer of a lactide and a glycolide. In another aspect, the polymer includes a poly(caprolactone). In yet another aspect, the polymer includes a poly(lactic acid), poly(L-lactide)/poly(D,L-Lactide) blends or copolymers of L-lactide and D,L-lactide. In yet another aspect, the polymer includes a copolymer of lactide and e-caprolactone. In yet another aspect, the polymer includes a polyester (e.g., a poly(lactide-co-glycolide). The poly(lactide-co-glycolide) may have a lactide:glycolide ratio ranges from about 20:80 to about 2:98, a lactide:glycolide ratio of about 10:90, or a lactide:glycolide ratio of about 5:95. In one aspect, the poly(lactide-co-glycolide) is poly(L-lactide-co-glycolide). Other examples of biodegradable materials include polyglactin, polyglycolic acid, autogenous, heterogenous, and xenogeneic tissue (e.g., pericardium or small intestine submucosa), and oxidized, regenerated cellulose. These meshes can be knitted, woven or non-woven meshes. Examples of non-woven meshes include electrospun materials.

Meshes and films may be prepared from non-biodegradable polymers.

Representative examples of non-biodegradable compositions include ethylene-co-vinyl acetate copolymers, acrylic-based and methacrylic-based polymers (e.g., poly(acrylic acid), poly(methylacrylic acid), poly(methylmethacrylate), poly(hydroxyethylmethacrylate), poly(alkylcynoacrylate), poly(alkyl acrylates), poly(alkyl methacrylates)), polyolefins such as poly(ethylene) or poly(propylene), polyamides (e.g., nylon 6,6), poly(urethanes) (e.g., poly(ester urethanes), poly(ether urethanes), poly(carbonate urethanes), poly(ester-urea)),polyesters (e.g., PET, polybutyleneterephthalate, and polyhexyleneterephthalate), polyethers (poly(ethylene oxide), poly(propylene oxide), poly(ethylene oxide)-poly(propylene oxide) copolymers, diblock and triblock copolymers, poly(tetramethylene glycol)), silicone containing polymers and vinyl-based polymers (polyvinylpyrrolidone, poly(vinyl alcohol), poly(vinyl acetate phthalate), poly(styrene-co-isobutylene-co-styrene), fluorine containing polymers (fluoropolymers) such as fluorinated ethylene propylene (FEP) or polytetrafluoroethylene (e.g., expanded PTFE).

The mesh or film material may comprise a combination of the above-mentioned biodegradable and non-degradable polymers. Further examples of polymers that may be used are either anionic (e.g., alginate, carrageenin, hyaluronic acid, dextran sulfate, chondroitin sulfate, carboxymethyl dextran, caboxymethyl cellulose and poly(acrylic acid)], or cationic [e.g., chitosan, poly-1-lysine, polyethylenimine, and poly(allyl amine)) (see generally, Dunn et al., J. Applied Polymer Sci. 50:353, 1993; Cascone et al., J. Materials Sci.: Materials in Medicine 5:770, 1994; Shiraishi et al., Biol. Pharm. Bull. 16:1164, 1993; Thacharodi and Rao, Int'l J. Pharm. 120:115, 1995; Miyazaki et al., Int'l J. Pharm. 118:257, 1995). Preferred polymers (including copolymers and blends of these polymers) include poly(ethylene-co-vinyl acetate), poly(carbonate urethanes), poly(hydroxyl acids) (e.g., poly(D,L-lactic acid) oligomers and polymers, poly(L-lactic acid) oligomers and polymers, poly(D-lactic acid) oligomers and polymers, poly(glycolic acid), copolymers of lactic acid and glycolic acid, copolymers of lactide and glycolide, poly(caprolactone), copolymers of lactide or glycolide and ε-caprolactone), poly(valerolactone), poly(anhydrides), copolymers prepared from caprolactone and/or lactide and/or glycolide and/or polyethylene glycol.

A variety of polymeric and non-polymeric films and meshes have been described which may be combined with an anti-scarring drug combination (or individual component(s) thereof). For example, the film or mesh may be a biodegradable polymeric matrix that conforms to the tissue and releases the agent in a controlled release manner. See, e.g., U.S. Pat. No. 6,461,640. The film or mesh may be a self-adhering silicone sheet which is impregnated with an antioxidant and/or antimicrobial. See, e.g., U.S. Pat. No. 6,572,878. The film or mesh may be a pliable shield with attachment ports and fenestrations that is adapted to cover a bony dissection in the spine. See, e.g., U.S. Pat. No. 5,868,745 and U.S. Patent Application No. 2003/0078588. The film or mesh may be a resorbable micro-membrane having a single layer of non-porous polymer base material of poly-lactide. See, e.g., U.S. Pat. No. 6,531,146 and U.S. Application No. 2004/0137033. The film or mesh may be a flexible neuro decompression device that has an outer surface texturized with microstructures to reduce fibroplasia when it is wrapped around a nerve in a canal. See, e.g., U.S. Pat. No. 6,106,558. The film or mesh may be a resorbable collagen membrane that is wrapped around the spinal chord to inhibit cell adhesions. See, e.g., U.S. Pat. No. 6,221,109. The film or mesh may be a wound dressing garment composed of an outer pliable layer and a self-adhesive inner gel lining which serves as a dressing for contacting wounds. See, e.g., U.S. Pat. No. 6,548,728. The film or mesh may be a bandage with a scar treatment pad with a layer of silicone elastomer or silicone gel. See, e.g., U.S. Pat. Nos. 6,284,941 and 5,891,076. The film or mesh may be a crosslinkable system with at least three reactive compounds each having a polymeric molecular core with at least one functional group. See, e.g., U.S. Pat. No. 6,458,889. The film or mesh may be composed of a prosthetic fabric having a 3-dimensional structure separating two surfaces in which one is open to post-surgical cell colonization and one is linked to a film of collagenous material. See, e.g., U.S. Pat. No. 6,451,032. The film or mesh may be composed by crosslinking two synthetic polymers, one having nucleophilic groups and the other having electrophilic groups, such that they form a matrix that may be used to incorporate a biologically active compound. See, e.g., U.S. Pat. Nos. 6,323,278; 6,166,130; 6,051,648 and 5,874,500. The film or mesh may be a film composed of hetero-bifunctional anti-adhesion binding agents that act to covalently link substrate materials, such as collagen, to receptive tissue. See, e.g., U.S. Pat. No. 5,580,923. The film or mesh may be a conformable warp-knit fabric of oxidized regenerated cellulose or other bioresorbable material which acts like a physical barrier to prevent postoperative adhesions. See, e.g., U.S. Pat. No. 5,007,916. Meshes for use in the practice of the invention also are described in U.S. Pat. No. 6,575,887, and co-pending application, entitled “Perivascular Wraps,” filed Sep. 26, 2003 (U.S. Ser. No. (U.S. Ser. No. 10/673,046).

In one aspect, the mesh may be suitable for use in hernia repair surgery or in other types of surgical procedures. Mesh fabrics for use in connection with hernia repairs are disclosed in U.S. Pat. Nos. 6,638,284; 5,292,328; 4,769,038 and 2,671,444. Surgical meshes may be produced by knitting, weaving, braiding, or otherwise forming a plurality of yams (e.g., monofilament or multifilament yams made of polymeric materials such as polypropylene and polyester) into a support trellis. Knitted and woven fabrics constructed from a variety of synthetic fibers and the use of the fabrics, in surgical repair are also discussed in U.S. Pat. Nos. 3,054,406; 3,124,136; 4,193,137; 4,347,847; 4,452,245; 4,520,821; 4,633,873; 4,652,264; 4,655,221; 4,838,884 and 5,002,551 and European Patent Application No. 334,046. Implantable hernia meshes are described in U.S. Pat. Nos. 6,610,006; 6,368,541 and 6,319,264. Hernia meshes for the repair of hiatal hernias are described in, e.g., U.S. Pat. No. 6,436,030. Hernia meshes for the repair of abdominal (e.g., ventral and umbilical) hernias are described in U.S. Pat. No. 6,383,201. Infection-resistant hernia meshes are described in, e.g., U.S. Pat. No. 6,375,662. Hernia meshes such as those described in the patents listed above are suitable for combining with a fibrosis-inducing agent to create a mesh which promotes the growth of fibrous tissue.

In one aspect, the fibrosis-inhibiting drug combination (or individual component(s) thereof) can be incorporated into a biodegradable or dissolvable film or mesh that is then applied to the treatment site prior or post implantation of the prosthesis/implant. Exemplary materials for the manufacture of these films or meshes are hyaluronic acid (crosslinked or non-crosslinked), cellulose derivatives (e.g., hydroxypropyl cellulose), PLGA, collagen and crosslinked poly(ethylene glycol).

The film or mesh may be in the form of a tissue graft, which may be an autograft, allograft, biograft, biogenic graft or xenograft. Tissue grafts may be derived from various tissue types. Representative examples of tissues that may be used to prepare biografts include, but are not limited to, rectus sheaths, peritoneum, bladder, pericardium, veins, arteries, diaphragm and pleura. The biograft may be harvested from a host, loaded with an anti-scarring drug combination (or individual component(s) thereof) and then applied in a perivascular manner at the site where lesions and intimal hyperplasia can develop (e.g., at an anastomotic site). Once implanted, the drug combination (e.g., amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate) is released from the graft and can penetrate the vessel wall to prevent the formation of intimal hyperplasia at the treatment site. In certain embodiments, the biograft may be used as a backing layer to enclose a composition (e.g., a gel or paste loaded with anti-scarring agent).

Films and meshes, which may be combined with drug combinations (or individual components thereof)according to the present invention, include commercially available products. Examples of films and meshes into which an anti-fibrosis drug combination (or individual component(s) thereof) can be incorporated include INTERCEED (Johnson & Johnson, Inc.), PRECLUDE (W.L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson. Another mesh is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the fibrosis-inducing agent may be applied only to the uncoated side of SEPRAMESH and not to the sodium hyaluronate/ carboxymethylcellulose coated side. Other films and meshes include: (a) BARD MARLEX mesh (C.R. Bard, Inc.), which is a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N.H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3, 4 & 5 and HERNIAMESH plugs T1, T2, and T3 from Herniamesh USA, Inc. (Great Neck, N.Y.).

Other examples of commercially available meshes which may be combined with fibrosis-inhibiting drug combinations (or individual components thereof) are described below. One example includes a prosthetic polypropylene mesh with a bioresorbable coating sold under the trade name SEPRAMESH Biosurgical Composite (Genzyme Corporation). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the fibrosis-inducing drug combination (or individual component(s) thereof) may be applied only to the uncoated side of SEPRAMESH and not to the sodium hyaluronate/carboxymethylcellulose coated side. Boston Scientific Corporation sells the TRELEX NATURAL Mesh which is composed of a unique knitted polypropylene material. Ethicon, Inc. makes the absorbable VICRYL (polyglactin 910) meshes (knitted and woven) and MERSILENE Polyester Fiber Mesh. Dow Coming Corporation (Midland, Mich.) sells a mesh material formed from silicone elastomer known as SILASTIC Rx Medical Grade Sheeting (Platinum Cured). United States Surgical/Syneture (Norwalk, Conn.) sells a mesh made from absorbable polyglycolic acid under the trade name DEXON Mesh Products. Membrana Accurel Systems (Obernburg, Germany) sells the CELGARD microporous polypropylene fiber and membrane. Gynecare Worldwide, a division of Ethicon, Inc. sells a mesh material made from oxidized, regenerated cellulose known as INTERCEED TC7. Integra LifeSciences Corporation (Plainsboro, N.J.) makes DURAGEN PLUS Adhesion Barrier Matrix, which can be used as a barrier against adhesions following spinal and cranial surgery and for restoration of the dura mater. HYDROSORB Shield from MacroPore Biosurgery, Inc. (San Diego, Calif.) is a film for temporary wound support to control the formation of adhesions in specific spinal applications.

Numerous polymeric and non-polymeric carrier systems that can be used with films and meshes have been described above. Methods for incorporating a fibrosis-inhibiting drug combination (or individual component(s) thereof) or a composition comprising a fibrosis-inhibiting drug combination (or individual component(s) thereof) onto or into the film or mesh include: (a) affixing (directly or indirectly) to the film or mesh a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) incorporating or impregnating into the film or mesh a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier (c) by coating the film or mesh with a substance such as a hydrogel which will in turn absorb a fibrosis-inhibiting drug combination (or individual component(s) thereof) or a composition comprising a fibrosis-inhibiting drug combination (or individual component(s) thereof), (d) constructing the film or mesh itself or a portion of the film or mesh with a fibrosis-inhibiting drug combination (or individual component(s) thereof) or a composition comprising a fibrosis-inhibiting drug combination (or individual component(s) thereof), or (e) by covalently binding a fibrosis-inhibiting drug combination (or individual component(s) thereof) or a composition comprising a fibrosis-inhibiting drug combination (or individual component(s) thereof) directly to the film or mesh surface or to a linker (small molecule or polymer) that is coated or attached to the film or mesh surface. For devices that are coated, the coating process can be performed in such a manner as to (a) coat only one surface of the film or mesh or (b) coat all or parts of both sides of the film or mesh.

The drug combination (or individual component(s) thereof) may be an integral part of the film or mesh (i.e., may reside within the fibers of the mesh). The fibrosis inhibiting drug combination (or individual component(s) thereof) can be incorporated directly into the film or mesh or it can be incorporated into a secondary carrier (polymeric or non-polymeric), as described above, that is then incorporated into the film or mesh.

The film or mesh may be coated with a fibrosis-inhibiting drug combination (or individual component(s) thereof) or a composition that includes the fibrosis-inhibiting drug combination (or individual component(s) thereof). In some embodiments, the composition is a polymer composition can function as a surgical adhesion barrier. The coating may take the form of a surface-adherent coating, mask, film, gel, foam, or mold.

A variety of polymeric compositions have been described that may be used in conjunction with the films and meshes of the invention. Such compositions may be in the form of, for example, gels, sprays, liquids, and pastes, or may be polymerized from monomeric or prepolymeric constituents in situ. For example, the composition may be a polymeric tissue coating which is formed by applying a polymerization initiator to the tissue and then covering it with a water-soluble macromer that is polymerizable using free radical initiators under the influence of UV light. See, e.g., U.S. Pat. Nos. 6,177,095 and 6,083,524. The composition may be an aqueous composition including a surfactant, pentoxifylline and a polyoxyalkylene polyether. See, e.g., U.S. Pat. No. 6,399,624. The composition may be a hydrogel-forming, self-solvating, absorbable polyester copolymers capable of selective, segmental association into compliant hydrogels mass upon contact with an aqueous environment. See, e.g., U.S. Pat. No. 5,612,052. The composition may be composed of fluent pre-polymeric material that is emitted to the tissue surface and then exposed to activating energy in situ to initiate conversion of the applied material to non-fluent polymeric form. See, e.g., U.S. Pat. Nos. 6,004,547 and 5,612,050. The composition may be composed of a gas mixture of oxygen present in a volume ratio of 1 to 20%. See, e.g., U.S. Pat. No. 6,428,500. The composition may be composed of an anionic polymer having an acid sulfate and sulfur content greater than 5% which acts to inhibit monocyte or macrophage invasion. See, e.g., U.S. Pat. No. 6,417,173. The composition may be composed of a non-gelling polyoxyalkylene composition with or without a therapeutic agent. See, e.g., U.S. Pat. No. 6,436,425. The composition may be coated onto tissue surfaces and may be composed of an aqueous solution of a hydrophilic, polymeric material (e.g., polypeptides or polysaccharide) having greater than 50,000 molecular weight and a concentration range of 0.01% to 15% by weight. See, e.g., U.S. Pat. No. 6,464,970.

Other representative examples of polymeric compositions which may be coated onto the film or mesh include poly(ethylene glycol)-based systems, hyaluronic acid and crosslinked hyaluronic acid compositions. These compositions can be applied as the final composition or they can be applied as materials that form crosslinked gel in situ.

Other compositions that can be used in conjunction with films and meshes, include, but are not limited to: (a) sprayable PEG-containing formulations such as COSEAL, SPRAYGEL, FOCALSEAL or DURASEAL; (b) hyaluronic acid-containing formulations such as RESTYLANE, HYLAFORM, PERLANE, SYNVISC, SEPRAFILM, SEPRACOAT, INTERGEL, (c) polymeric gels such as REPEL or FLOWGEL, (d) dextran sulfate gels such as the ADCON range of products, (e) lipid based compositions such as ADSURF (Brittania Pharmaceuticals).

The film or mesh (or device comprising the film or mesh) may be made sterile either by preparing them under aseptic environment and/or they may be terminally sterilized using methods known in the art, such as gamma radiation or electron beam sterilization methods or a combination of both of these methods.

Films and meshes may be applied to any bodily conduit or any tissue that may be prone to the development of fibrosis or intimal hyperplasia. Prior to implantation, the film or mesh may be trimmed or cut from a sheet of bulk material to match the configuration of the widened foramen, canal, or dissection region, or at a minimum, to overlay the exposed tissue area. The film or mesh may be bent or shaped to match the particular configuration of the placement region. The film or mesh may also be rolled in a cuff shape or cylindrical shape and placed around the exterior periphery of the desired tissue. The film or mesh may be provided in a relatively large bulk sheet and then cut into shapes to mold the particular structure and surface topography of the tissue or device to be wrapped. Alternatively, the film or mesh may be pre-shaped into one or more patterns for subsequent use. The films and meshes may be typically rectangular in shape and be placed at the desired location within the surgical site by direct surgical placement or by endoscopic techniques. The film or mesh may be secured into place by wrapping it onto itself (i.e., self-adhesive), or by securing it with sutures, staples, sealant, and the like. Alternatively, the film or mesh may adhere readily to tissue and therefore, additional securing mechanisms may not be required.

The films or meshes of the invention may be used for a variety of indications, including, without limitation: (a) prevention of surgical adhesions between tissues following surgery (e.g., gyneacologic surgery, vasovasostomy, hernia repair, nerve root decompression surgery and laminectomy); (b) prevention of hypertrophic scars or keloids (e.g., resulting from tissue burns or other wounds); (c) prevention of intimal hyperplasia and/or restenosis (e.g., resulting from insertion of vascular grafts or hemodialysis access devices); or (d) may be used in affiliation with devices and implants that lead to scarring as described herein (e.g., as a sleeve or mesh around a breast implant to reduce or inhibit scarring).

In one embodiment, films or meshes may be used to prevent adhesions that occur between tissues following surgery, injury or disease. Adhesion formation, a complex process in which bodily tissues that are normally separate grow together, occurs most commonly as a result of surgical intervention and/or trauma. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation can be inhibited. Adhesion formation or unwanted scar tissue accumulation and encapsulation complicates a variety of surgical procedures and virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule which compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies, infections (fungal, mycobacterium). Surgical procedures that may lead to surgical adhesions may include cardiac, spinal, neurologic, pleural, thoracic and gynaecologic surgeries. However, adhesions may also develop as a result of other processes, including, but not limited to, non-surgical mechanical injury, ischemia, hemorrhage, radiation treatment, infection-related inflammation, pelvic inflammatory disease and/or foreign body reaction. This abnormal scarring interferes with normal physiological functioning and, in come cases, can force and/or interfere with follow-up, corrective or other surgical operations. For example, these post-operative surgical adhesions occur in 60 to 90% of patients undergoing major gynaecologic surgery and represent one of the most common causes of intestinal obstruction in the industrialized world. These adhesions are a major cause of failed surgical therapy and are the leading cause of bowel obstruction and infertility. Other adhesion-treated complications include chronic pelvic pain, urethral obstruction and voiding dysfunction.

Currently, preventative therapies, administered 4 to 5 days following surgery, are used to inhibit adhesion formation. Various modes of adhesion prevention have been examined, including (1) prevention of fibrin deposition, (2) reduction of local tissue inflammation, and (3) removal of fibrin deposits. Fibrin deposition is prevented through the use of physical adhesion barriers that are either mechanical or comprised of viscous solutions. Although many investigators are utilizing adhesion prevention barriers, a number of technical difficulties exist.

In one aspect, the present invention provides films and meshes that include an anti-scarring drug combination (or individual component(s) thereof) or a composition that includes an anti-scarring drug combination (or individual component(s) thereof) for use as surgical adhesion barriers.

In one aspect, films and meshes may be used to prevent surgical adhesions in the epidural and dural tissue which is a factor contributing to failed back surgeries and complications associated with spinal injuries (e.g., compression and crush injuries). Scar formation within dura and around nerve roots has been implicated in rendering subsequent spine operations technically more difficult. To gain access to the spinal foramen during back surgeries, vertebral bone tissue is often disrupted. Back surgeries, such as laminectomies and diskectomies, often leave the spinal dura exposed and unprotected. As a result, scar tissue frequently forms between the dura and the surrounding tissue. This scar is formed from the damaged erector spinae muscles that overlay the laminectomy site. This results in adhesion development between the muscle tissue and the fragile dura, thereby, reducing mobility of the spine and nerve roots which leads to pain and slow post-operative recovery. To circumvent adhesion development, a scar-reducing barrier may be inserted between the dural sleeve and the paravertebral musculature post-laminotomy. This reduces cellular and vascular invasion into the epidural space from the overlying muscle and exposed cancellous bone and thus, reduces the complications associated with the canal housing the spinal chord and/or nerve roots.

In another aspect, films and meshes comprising an anti-scarring drug combination (or individual component(s) thereof) may be used to prevent the fibrosis from occurring between a hernia repair mesh and the surrounding tissue. Hernias are abnormal protrusions (outpouchings) of an organ or other body structure through a defect or natural opening in a covering membrane, muscle or bone. Hernias themselves are not dangerous, but can become extremely problematic if they become incarcerated. Surgical prostheses used in hernia repair (referred to herein as “hernia meshes”) include prosthetic mesh-or gauze-like materials, which support the repaired hernia or other body structures during the healing process. Hernias are often repaired surgically to prevent complications. Conditions in which a hernia mesh may need to be used include, without limitation, the repair of inguinal (i.e., groin), umbilical, ventral, femoral, abdominal, diaphragmatic, epigastric, gastroesophageal, hiatal, intermuscular, mesenteric, paraperitoneal, rectovaginal, rectocecal, uterine, and vesical hernias. Hernia repair typically involves returning the viscera to its normal location and the defect in the wall is primarily closed with sutures, but for bigger gaps, a mesh is placed over the defect to close the hernia opening. Inclusion of a fibrosis-inhibiting drug combination (or individual component(s) thereof) or a composition comprising a fibrosis-inhibiting drug combination (or individual component(s) thereof) into or onto a hernia repair mesh may reduce or prevent fibrosis proximate to the implanted hernia mesh, thereby minimizing the possibility of adhesions between the abdominal wall or other tissues and the mesh itself, and reducing further complications and abdominal pain.

In yet another aspect, films or meshes may be used to prevent hypertrophic scars or keloids (e.g., resulting from tissue burns or other wounds). Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. A film or mesh that comprises an anti-scarring drug combination (or individual component(s) thereof) or a composition that comprises an anti-scarring drug combination (or individual component(s) thereof) may be placed in contact with a wound or burn site in order to prevent formation of hypertrophic scar or keloids.

In yet another aspect, films and meshes are provided that may be used for delivering an anti-scarring drug combination (or individual component(s) thereof) to an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, eustachian tubes, the external auditory mayal, vas deferens and fallopian tubes. Examples of cavities include the abdominal cavity, the buccal cavity, the peritoneal cavity, the pericardial cavity, the pelvic cavity, perivisceral cavity, pleural cavity and uterine cavity.

Examples of conditions that may be treated or prevented with fibrosis-inhibiting films and meshes include iatrogenic complications of arterial and venous catheterization, complications of vascular dissection, complications of gastrointestinal passageway rupture and dissection, restonotic complications associated with vascular surgery (e.g., bypass surgery), and intimal hyperplasia.

In one aspect, an anti-scarring drug combination (or individual component(s) thereof) may be delivered from a film or mesh to the external walls of body passageways or cavities for the purpose of preventing and/or reducing a proliferative biological response that may obstruct or hinder the optimal functioning of the passageway or cavity, including, for example, iatrogenic complications of arterial and venous catheterization, aortic dissection, cardiac rupture, aneurysm, cardiac valve dehiscence, graft placement (e.g., A-V-bypass, peripheral bypass, CABG), fistula formation, passageway rupture and surgical wound repair.

The films or meshes may be used in the form of a perivascular wrap to prevent restenosis at anastomotic sites resulting from insertion of vascular grafts or hemodialysis access devices. In this case, perivascular wraps may be incorporated with or coated with a fibrosis-inhibiting drug combination (or individual component(s) thereof), which can be used in conjunction with a vascular graft to inhibit scarring at an anastomotic site. These films or meshes may be placed or wrapped in a perivascular (periadventitial) manner around the outside of the anastomosis at the time of surgery. Film and mesh implants comprising an anti-scarring agent may be used with synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts).

In order to further the understanding of such conditions, representative complications leading to compromised body passageway or cavity integrity are discussed in more detail below.

Cardiac Bypass Surgery

Coronary artery bypass graft (“CABG”) surgery was introduced in the 1950s, and still remains a highly invasive, open surgical procedure, although less invasive surgical techniques are being developed. CABG surgery is a surgical procedure that is performed to overcome many types of coronary artery blockages. The purpose of bypass surgery is to increase the circulation and nourishment to the heart muscle that has been reduced due to arterial blockage. This procedure involves the surgeon accessing the heart and the diseased arteries, usually through an incision in the middle of the chest. Often, healthy arteries or veins are “harvested” from the patient to create “bypass grafts” that channel the needed blood flow around the blocked portions of the coronary arteries. The arteries or veins are connected from the aorta to the surface of the heart beyond the blockages thereby forming an autologous graft. This allows the blood to flow through these grafts and “bypass” the narrowed or closed vessel. The use of synthetic graft materials to create the “bypass” has been limited due to the lack of the appropriate biocompatibility of these synthetic grafts. CABG has significant short term limitations, including medical complications, such as stroke, multiple organ dysfunction, inflammatory response, respiratory failure and post-operative bleeding, each of which may result in death. Another problem associated with CABG is restenosis. Restenosis is typically defined as a renarrowing of an arterial blood vessel within six months of the CABG procedure. It typically occurs in approximately 25% to 45% of patients, and is the result of an excessive healing response to arterial injury after a revascularization procedure. Restenosis may occur within a short period following a procedure or may develop over the course of months or years. Longer term or “late” restenosis may result from excessive proliferation of scar tissue at the treatment site, the causes of which are not well understood. Thus any product that may reduce the incidence or magnitude of the restenotic process following CABG surgery can greatly enhance the well being of a patient.

In order to prevent the restenotic complications associated with CABG surgery, such as those discussed above, a wide variety of therapeutic agents (with or without a carrier) may be delivered to the external portion of the blood vessel. The carrier (e.g., polymer) or therapeutic agent/composition can be applied to the external portion of the vessel following the interventional or surgical procedure in order to prevent the restenotic complications.

Peripheral Bypass Surgery

Peripheral arterial disease (PAD) refers to diseases of any of the blood vessels outside of the heart. PAD is a range of disorders that may affect the blood vessels in the hands, arms, legs, or feet. The most common form of PAD is atherosclerosis. Atherosclerosis is a gradual process in which cholesterol and scar tissue build up in the arteries to form plaque. This build-up causes a gradual narrowing of the artery, which leads to a decrease in the amount of blood flow through that artery. When the flow of blood decreases, it results in a decrease of oxygen and nutrient supply to the body's tissues, which in turn may result in pain sensation. When the arteries to the legs are affected, the most common symptom is pain in the calf when walking. This is known as intermittent claudication.

Peripheral bypass surgery is a procedure to bypass an area of stenosed (narrowed) or blocked artery that is a result of atherosclerosis. In this surgical procedure, a synthetic graft (artificial blood vessels) or an autologous graft, vein, will be implanted to provide blood flow around the diseased area. First, the surgeon makes an incision in the leg, thigh, calf or ankle skin. The location of the incision may vary based on which vessels need to be bypassed and where there is healthy artery to connect to maintain the blood flow. The bypass graft is sewn into the artery above the stenosis or blockage, and below the stenosis or blockage. This bypass provides a means whereby blood will reach the tissue that has not been receiving enough blood and oxygen. Synthetic bypass grafts used in the legs are usually made of ePTFE.

Restenosis and occlusion of bypass grafts are one of the most important problems in peripheral bypass surgery. This restenosis is caused by neointimal growth (hyperplasia) and is especially pronounced within artificial graft material. This restenosis is usually at the anastomotic site where the graft and artery are connected via a surgical procedure. The intimal tissue typically grows from the native vessel into the graft. In order to prevent the restenotic complications associated with peripheral bypass surgery, such as those discussed above, a wide variety of therapeutic agents (with or without a carrier)/compositions may be delivered to the external portion of the blood vessel. The polymer or therapeutic agent/composition can be applied to the external portion of the vessel/anastomotic site following the interventional or surgical procedure in order to prevent the restenotic complications.

Arterio- Venous (AV) Fistula

The arterio-venous (AV) fistula is surgically created vascular connection which allows the flow of blood from an artery directly to a vein. The AV fistula was first created by researchers for kidney failure patients who must undergo kidney dialysis.

Hemodialysis requires a viable artery and vein to draw blood from and return it to the body. The repeated puncturing often either causes a vein or artery to fail or causes other complications for the patient. The AV fistula increases the amount of possible puncture sites for hemodialysis and minimizes the damage to the patient's natural blood vessels. The connection that is created between the vein and artery forms a large blood vessel that continuously supplies an increased blood flow for performing hemodialysis.

Restenosis and eventual occlusion are one of the most important problems in the long term patency of the AV fistula. In order to prevent the restenotic complications associated with the surgical formation of an AV fistula, a wide variety of therapeutic agents (with or without a carrier)/compositions may be delivered to the external portion of the blood vessel. The polymer or therapeutic agent/composition can be applied to the external portion of the vessel/anastomotic site following the interventional or surgical procedure in order to prevent the restenotic complications.

Arterio-Venous (AV) Graft Surgery

The AV graft surgical procedure is used for similar application as those for the AV fistula (e.g., hemodialysis patients). For the AV graft surgery, a synthetic graft material is used to connect the artery to the vein rather that the direct connection of the artery to the vein as is the case for the AV fistula. The incidence of intimal hyperplasia, which leads to occlusion of the graft, is one of the main factors that affect the long term patency of these grafts. This intimal hyperplasia may occur at the venous anastomosis and at the floor of the vein. A product that may reduce or prevent this occurrence of intimal hyperplasia will increase the duration of patency of these grafts. In order to reduce the occurrence of intimal hyperplasia at the venous anastomosis of an AV graft, a wide variety of therapeutic agents (with or without a carrier)/compositions may be delivered to the external portion of the blood vessel. The polymer or therapeutic agent/composition can be applied to the external portion of the vessel/anastomotic site following the interventional or surgical procedure in order to prevent the restenotic complications.

Anastomotic Closure Devices

Anastomotic closure devices provide a means for rapidly repairing an anastomosis. The use of some of these devices requires an invasive surgical procedure. In one embodiment of this invention, following the use of an anastomotic closure device, the mesh containing the therapeutic agent may be wrapped around the anastomosis and the anastomotic closure device, if it is left at the surgical site.

In one embodiment, the invention provides a method for treating or preventing intimal hyperplasia that includes delivering to an anastomotic site a delivery device. The device includes a therapeutic agent and a biodegradable polymer, wherein at least some of the biodegradable polymer is in the form of a mesh. Exemplary anastomotic sites include venous anastomosis, arterial anastomosis, arteriovenous fistula, arterial bypass, and arteriovenous graft. Preferably, the device includes a polymer mesh with a therapeutic agent is delivered to an external portion of an anastomotic site.

Transplant Applications

There are many applications in which various organs in the human body fail to function in a manner to sustain the well being of the patient. When an appropriate donor organ is available, an impaired organ may be replaced by a donor organ (e.g., lung, heart, kidney etc). One of the potential complications following these transplant surgeries is the potential for stenosis to occur in the blood vessels at or near the anastomotic site between the donor and recipient vessels. For example, transplant renal artery stenosis is a complication that may occur following a kidney transplant. Transplant renal artery stenosis is when the artery from the abdominal aorta to the kidney narrows, limiting blood flow to the kidney. This may also make it difficult to keep blood pressure under control. Treatment typically involves expanding the narrowed segment using a small balloon.

One method to treat this stenosis is to apply the composition of this invention around the anastomotic site (junction of the donor and recipient vessels) in a perivascular manner. In a similar manner, the composition of this invention may be applied in a peritubular manner to the exterior surfaces of the trachea and or bronchi following a lung transplant procedure.

According to the present invention, any anti-scarring drug combination (or individual component(s) thereof) described above can be utilized in the practice of this embodiment. Films and meshes may be adapted to contain and/or release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

As films and meshes are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in films or meshes include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the film or mesh, the exemplary anti-fibrosing drug combinations (or individual components thereof) should be administered under the following dosing guidelines. The total amount (dose) of anti-scarring agent(s) in or on the film or mesh may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with films or meshes in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Glaucoma Drainage Devices

In one aspect, the present invention provides for the combination of an anti-scarring drug combination (or individual component(s) thereof) and a glaucoma drainage device.

Various types of glaucoma drainage devices have been described. Some glaucoma drainage devices include a plate and a tube. The function of the tube is to deliver aqueous from within the eye onto the upper surface of the episcleral plate. The episcleral plate is firmly sutured to the sclera and covered by a thick flap of Tenon's tissue and conjunctiva. The function of the plate is to initiate the formation of a large circular bleb which develops a specialized fibrovascular bleb lining and becomes distended by aqueous. It is this fibrovascular bleb lining which is responsible for regulating the escape of aqueous from the eye and which determines the final level of intraocular pressure (IOP) that is achieved after insertion of the implant. If the fibrovascular response is too great, the drainage capability of the device is reduced. In an embodiment of the present invention, a fibrosis-inhibiting drug combination (or individual component(s) thereof) is incorporated into or onto all or a portion of the device such that the released fibrosis-inhibiting drug combination (or individual component(s) thereof) modulates the healing response, thereby enabling the device to function correctly.

Glaucoma drainage devices may be, for example, a conduit attached to an episcleral drainage plate having a porous posterior surface for cellular ingrowth and attachment by the sclera. See, e.g., U.S. Pat. No. 5,882,327. The glaucoma drainage device may be composed of a foldable and rollable episcleral plate and a drainage tube whereby the device may be delivered to the implant site through an injection delivery system. See, e.g., U.S. Pat. No. 6,589,203. The glaucoma drainage device may be pressure regulator composed of a base plate formed of a thin, flexible rubber material (e.g., silicone rubber) which has a mounted housing chamber that is attached to a tube. See, e.g., U.S. Pat. No. 5,752,928. The glaucoma drainage device may be composed of an elastomeric plate having a sealing member that conforms to the sclera to restrict fluid and an attached non-valved elastomeric drainage tube. See, e.g., U.S. Pat. No. 5,476,445. The glaucoma drainage device may be composed of ridged plates that extend outwardly that are concave on one side to match the curvature of the sclera and are adapted for side by side attachment to the sclera whereby a tube extends between the ridged plates for communication. See, e.g., U.S. Pat. No. 4,457,757. The glaucoma drainage device may be composed of a thin, elliptical, elastomeric plate having a centrally positioned hole for growth of scar tissue and an elastomeric drainage tube attached to the plate for fluid communication with the eye. See, e.g., U.S. Pat. No. 5,397,300. The glaucoma drainage device may be composed of a tube with a circumferential hole with a connected disk at the outlet end of the tube for placing on a surface of an eyeball. See, e.g., U.S. Pat. No. 5,868,697. The glaucoma drainage device may be a tube with a flow controlling structure that constricts flow passage within the tube and has at least one circumferential hole within the tube that is temporarily occluded with an absorbable material. See, e.g., U.S. Pat. No. 6,203,513. The glaucoma drainage device may be composed of a tube with an engagement means and a porous, liquid-absorbing plug with an attached filamentary extension that substantially restricts fluid flow. See, e.g., U.S. Pat. No. 5,300,020. The glaucoma drainage device may be a resilient polymeric drain implant with a passage extending between the ends and flanges that project radially from the body. See, e.g., U.S. Pat. No. 4,968,296. The glaucoma drainage device may be a shunt to divert aqueous humor in the eye from the anterior chamber into a portion of the device that branches to provide fluid communication in either direction along the Schlemm's canal. See, e.g., U.S. Pat. No. 6,626,858.

Glaucoma drainage devices, which may be combined with anti-scarring drug combinations (or individual components thereof) according to the present invention, include commercially available products. For example, cylindrical tubes, such as the AQUAFLOW Collagen Glaucoma Drainage Device (STAAR Surgical Company, Monrovia, Calif.) may be used in the practice of the present invention. Other examples of glaucoma drainage devices includes the Molteno Glaucoma Implant (Single Plate Molteno Implant, Pressure Ridge Single Plate Molteno Implant (D1), Microphthalmic Plate Molteno Implant (M1), Double Plate Molteno Implant (R2/L2), and Pressure Ridge Double Plate Molteno Implant (DR2/DL2) from Molteno Opthalmic Limited (New Zealand), BAERVELDT Glaucoma Implants (Models BG-101-350, BG-102-350, BG-103-250; Pfizer, New York, N.Y.), and the Ahmed Glaucoma Valve (Models FP7, S2, S3, PS2, PS3, B1 from New World Medical, Inc. (Rancho Cucamonga, Calif.).

In one aspect, the present invention provides a glaucoma drainage device that includes an anti-scarring drug combination (or individual component(s) thereof) or a composition that includes an anti-scarring drug combination (or individual component(s) thereof). Numerous polymeric and non-polymeric delivery systems for use in glaucoma drainage devices have been described above. Methods for incorporating a fibrosis-inhibiting drug combination (or individual component(s) thereof) or a composition comprising a fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) by inserting the device into a sleeve or mesh which is comprised of or coated with a fibrosis-inhibiting drug combination (or individual component(s) thereof) or a composition comprising a fibrosis-inhibiting drug combination (or individual component(s) thereof), (f) constructing the device itself or a portion of the device with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface.

In addition to coating the device with a fibrosis-inhibiting drug combination (or individual component(s) thereof) or a composition comprising a fibrosis-inhibiting drug combination (or individual component(s) thereof), a fibrosis-inhibiting drug combination (or individual component(s) thereof) or a composition comprising a fibrosis-inhibiting drug combination (or individual component(s) thereof) can be mixed with the materials that are used to make the device such that the fibrosis-inhibiting drug combination (or individual component(s) thereof) or the composition comprising a fibrosis-inhibiting drug combination (or individual component(s) thereof) is incorporated into the final device.

In one embodiment, the methods above can be used to incorporate the fibrosis-inhibiting drug combination (or individual component(s) thereof) into or onto all or portions of the plate of the device.

In another embodiment, the methods above can be used to incorporate the fibrosis-inhibiting drug combination (or individual component(s) thereof) into or onto all or portions of the tube of the device.

In yet another embodiment, the methods above can be used to incorporate the fibrosis-inhibiting drug combination (or individual component(s) thereof) into or onto all or potions of both the plate and the tube of the device.

In addition to incorporation of a fibrosis-inhibiting drug combination (or individual component(s) thereof) into or onto the device (e.g., as a coating), another biologically active agent can be incorporated into or onto the device, for example an anti-inflammatory (e.g., dexamethazone or aspirin) or a MMP inhibitor.

According to the present invention, any fibrosis-inhibiting drug combination (or individual component(s) thereof) described above can be utilized in the practice of this embodiment. Within one embodiment of the invention, glaucoma drainage devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As glaucoma drainage devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in glaucoma drainage devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the devices, the exemplary anti-fibrosing agents, used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with glaucoma drainage devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Prosthetic Heart Valves

The present invention provides for the combination of a drug combination (or individual component(s) thereof) and a prosthetic heart valve.

Prosthetic heart valves are devices that are used to replace natural heart valves that are defective, due to congenital malformations, infections, partial occlusion, or wearing. Prosthetic heart valves are typically composed of an occluder(s) attached to the occluder base, which is in turn attached to the suture ring that provides anchorage of the device to the heart tissue. The occluder base is annular and provides a passageway for blood flow. There may be one or more occluders which alternate in an opened and closed position to regulate the flow of blood. To secure the prosthetic heart valve to the heart tissue, a suture ring, typically composed of a knit fabric tube, is rolled into a toroidal form and is secured to the periphery of the occluder base of the prosthesis. Affixing the suture ring to the heart tissue typically occurs using sutures, sealants, adhesives, staples, or clamping with metal or polymer wires.

Although the design of prosthetic heart valves has been gradually refined, complications continue to occur. Since the suture rings are often made out of synthetic material, thrombus, fibrosis and pannus often occur around the prosthetic heart valve. This scar formation often hinders the function of the valve and over time may require a second surgical procedure and replacement. Suture rings are generally composed of synthetic polymer, including, but not limited to, polyester (e.g., DACRON), polytetrafluoroethylene (e.g., TEFLON), silicone, and polypropylene. Suture rings are often made of a filler material with a woven material stitched over the filler. The surface of the suture ring is often course due to the covering cloth material. This predisposes the suture ring to scarring formation early in the post-operative period with severe pannus/fibrosis developing over several months following implantation. The consequences of fibrosis encroachment onto a prosthetic heart valve can be drastic, and potentially catastrophic. For example, fibrosis may inhibit valve occluder function by limiting its ability to open and close properly. The fibrosis may extend from the suture ring to the leaflets. This fibrosis may fuse the leaflets at their commissure, distort individual leaflets, and/or stiffen leaflets such that they do not open or close properly. The end result of this fibrosis typically is a heart valve that is both stenotic and insufficient.

There are two main types of prosthetic heart valves, mechanical and bioprosthetic. Typically, both mechanical and bioprosthetic heart valves utilize a synthetic suture ring. They differ primarily in the type of occluder that is utilized. The occluders of the mechanical heart valve may be composed of a ball and cage assembly, single leaflet disk valves, or bileaflet disk valves. The occluders of the bioprosthetic heart valve are composed of animal or human tissue that mimic the appearance and function of the natural heart valve it is replacing. The bioprosthetic heart valve leaflets are usually composed of chemically treated tissue. The harvested valves are fixed in glutaraldehyde or similar fixatives in order to make them suitable for human implantation.

In one aspect, the prosthetic heart valve may be a mechanical prosthesis which is typically composed of rigid leaflets formed of a biocompatible substance (e.g., pyrolitic carbon, titanium or DACRON). Mechanical prosthetic heart valves may be a ball and cage assembly, bileaflet, trileaflet or tilting disks. The most common is the bileaflet type since the hemodynamics of this valve is better as blood flow is smoother and less turbulent. For example, the mechanical prosthesis may be composed of a base with an external suture ring and an internal rim for blood flow as well as at least two closing leaflets. See, e.g., U.S. Pat. No. 6,068,657. The mechanical prosthesis may be composed of annular valve housing with a center orifice and first and second valve leaflets pivotally mounted to the valve housing. See, e.g., U.S. Pat. Nos. 4,808,180 and 5,026,391. The mechanical prosthesis may be designed with an annular body with at least one leaflet pivotally mounted such that it is movable between an open and closed position by a magnet that exerts a force on the leaflet at a defined pressure. See, e.g., U.S. Pat. No. 6,638,303. The mechanical prosthesis may have an annular body with a plurality of hinges which form an entrance ramp and supports at least one leaflet to the valve body. See, e.g., U.S. Pat. Nos. 6,645,244 and 5,919,226. The mechanical prosthesis may be composed of a supporting flexible, cylindrical frame with a cover that forms a cusp supporting stent for the valve trileaflet apparatus and a sewing ring as an attachment surface. See, e.g., U.S. Pat. No. 5,258,023. The mechanical prosthesis may have an increased valve lumen composed of a single piece valve orifice housing with at least one movable occluder coupled to the housing and a suture cuff for attaching the housing to the heart tissue. See, e.g., U.S. Pat. Nos. 6,007,577 and 6,391,053. The mechanical prosthesis may be composed of a sewing ring and a removable valve assembly which slides in a central core of the sewing ring. See, e.g., U.S. Pat. No. 5,032,128. The mechanical prosthesis may be a highly flexible cylindrical stent composed of a plurality of separate adjacent stent members with alternating cusps and commissures that are able to move radially and support a plurality of flexible leaflets. See, e.g., U.S. Pat. Nos. 6,558,418 and 6,338,740. Other mechanical heart valve prostheses are described in, e.g., U.S. Pat. Nos. 6,395,025; 6,358,278; 6,176,877; 6,139,575 and 5,984,958.

In another aspect, the prosthetic heart valve may be a bioprosthetic device which typically is flexible leaflets formed of a biological material (e.g., porcine valves or bovine pericardial valves). Tissue valves may be supported with a stent frame that provides the leaflets with more structure and durability. Stentless tissue valves may also be implanted by harvesting the porcine valves with the pig's aorta still attached. For example, the bioprosthetic heart valve, which may be obtained from a donor (e.g., porcine), may be treated to reduce antigens to prevent inflammatory response upon transplantation. See, e.g., U.S. Pat. No. 6,592,618. The bioprosthetic heart valve may be composed of a biological tissue material disposed around a mechanical annular support to provide at least part of the sewing ring. See, e.g., U.S. Pat. No. 6,582,464. The bioprosthetic heart valve may be composed of a xenograft mitral valve (e.g., porcine) and a sewing tube and cover of flexible material which is attached to the mitral valve. See, e.g., U.S. Pat. No. 5,662,704. The bioprosthetic heart valve may be composed of a natural tissue heart valve attached to a prosthetic stent frame that may be covered by a fabric cover. See, e.g., U.S. Pat. Nos. 3,983,581; 4,035,849; 5,861,028; 6,350,282 and 6,585,766. The bioprosthetic heart valve may be a self-supporting stentless valve that may be composed of a tubular body of mammalian origin. See, e.g., U.S. Pat. Nos. 5,156,621 and 6,342,070.

In another aspect, the prosthetic heart valve may be inserted into place using minimally-invasive techniques. For example, the prosthetic heart valve may be an expandable device adapted for delivery in a collapsed state to an implantation site and then expanded to a plurality of leaflets attached to a stent system. See, e.g., U.S. Pat. No. 6,454,799.

In another aspect, the device may be a component of the heart valve. For example, the device may be an implantable annular ring for receiving a prosthetic heart valve. See, e.g., U.S. Pat. No. 6,106,550. The device may be a suture ring having an outer peripheral tapered thread for attaching a heart valve prosthesis. See, e.g., U.S. Pat. No. 6,113,632. The device may be a suture ring for a mechanical heart valve composed of a stiffening ring attachment, a knit fabric sewing cuff and a locking ring. See, e.g., U.S. Pat. No. 5,071,431.

Prosthetic heart valves and components thereof (e.g., annular suture rings), which may be combined with one or more drugs according to the present invention, include commercially available products, such as the Carpentier-Edwards PERIMOUNT (CEP) Pericardial Bioprosthesis, Carpentier-Edwards S.A.V. Aortic Bioprosthesis and Edwards PRIMA PLUS STENTLESS BIOPROSTHESIS from Edwards Lifesciences (Irvine, Calif.), the SJM REGENT Valve from St. Jude Medical (St. Paul, Minn.), and the MOSAIC Bioprosthetic Heart Valve from Medtronic (Minneapolis, Minn.).

In one aspect, the present invention provides prosthetic heart valve devices that include a fibrosis-inhibiting drug combination (or individual component(s) thereof) or a composition comprising a fibrosis-inhibiting drug combination (or individual component(s) thereof). Numerous polymeric and non-polymeric delivery systems for use in prosthetic heart valves have been described above. Methods for incorporating a fibrosis-inhibiting drug combination (or individual component(s) thereof) or a composition comprising a fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface, and/or (g) any combination of the aforementioned.

According to the present invention, any fibrosis-inhibiting drug combination (or individual component(s) thereof) described above can be utilized in the practice of this embodiment. Within one embodiment of the invention, prosthetic heart valves may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As prosthetic heart valve devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in prosthetic heart valves include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the prosthetic heart valve, the exemplary anti-fibrosing drug combinations (or individual components thereof), used alone or in combination, should be administered under the following dosing guidelines. The total amount (dose) of anti-scarring agent(s) in or on the prosthetic heart valve may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with prosthetic heart valve devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Penile Implants

In one aspect, the present invention provides for the combination of an anti-scarring drug combination (or individual component(s) thereof) and a penile implant device. In one aspect, penile implants are loaded with an anti-scarring drug combination (or individual component(s) thereof) or a composition that includes an anti-scarring drug combination (or individual component(s) thereof) to prevent fibrous encapsulation.

Penile implants are used to treat erectile dysfunction and are generally flexible rods, hinged rods or inflatable devices with a pump. Penile implants may be composed of rods, coils, inflatable tubes and/or pressure chambers and may be used to provide erectile function, enlargement or provide shape to a misshapen or damaged penis. For example, the penile implant may be an implantable polymeric material which is injected into the lamina propria mucosae of the glans in order to enlarge the glans of the male genital organ. See, e.g., U.S. Pat. No. 6,418,934. The penile implant may be composed of a pair of arced, elongated portions made of silicone rubber that are mirror images of each other, which has a varying circumferential wall thickness. See, e.g., U.S. Pat. No. 6,537,204. The penile implant may be used to increase penile volume by being adapted to cover the outer lateral sides of the corpus cavemosum without covering the upper and lower sides thereof. See, e.g., U.S. Pat. No. 6,015,380. The penile implant may be an inflatable, self-contained implant composed of a cylindrical body having a pump that transfers fluid from a reservoir to a pressure chamber that has a pressure relief valve. See, e.g., U.S. Pat. Nos. 4,898,158 and 4,823,779. The penile implant may be composed of an elongated rod having a relatively short proximal stem portion, which is covered by a layer of hydrophilic material that contains a plurality of openings and swells as it absorbs water. See, e.g., U.S. Pat. No. 4,611,584. The penile implant may be composed of at least one inflatable tube that has fluid interchange with a mounting base which is controlled by a manual pump implanted in the scrotum. See, e.g., U.S. Pat. No. 6,475,137. The penile implant may be a flexible double-walled partial cylindrical sleeve that has bellow-like construction which is suited for penile malformation. See, e.g., U.S. Pat. No. 5,669,870. The penile implant may be used for correcting erectile impotence by being composed of at least one flexible portion with a pressure chamber connected by tubing to an accumulator charged with fluid, such that pressurizing fluid flows when the valve is opened. See, e.g., U.S. Pat. No. 4,917,110. The penile implant may be composed of a stainless steel pad supported by a plurality of strands which is surrounded by a cylinder with a silicone ring that can move longitudinally in response to the expansion or shrinkage of the penis. See, e.g., U.S. Pat. No. 5,433,694. The penile implant may increase girth and length by being composed of a cylindrical sleeve that has an elastic outer sheet and an inner inelastic sheet that forms a closed sack to receive a fluid under pressure from a fluid source. See, e.g., U.S. Pat. No. 5,445,594. The penile implant may be composed of a braided sleeve with an outer elastomeric surface and inner surface having grooves and ribs in a helical arrangement, such that the implant is malleable having both a bendable configuration and an unbent rigid configuration. See, e.g., U.S. Pat. No. 5,512,033. The penile implant may be a polymeric matrix having dissociated cartilage-forming cells deposited on and in said matrix whereby a cartilaginous structure is formed upon implantation having controlled biomechanical properties and tensile strength. See, e.g., U.S. Pat. No. 6,547,719. The penile implant may be composed of an implantable supply pump, deformable reservoir, and conducting/dispensing catheters, such that a vasodilator agent is delivered to the erectile bodies to treat male impotence. See, e.g., U.S. Pat. No. 6,679,832. Other penile implants are described in, e.g., U.S. Pat. Nos. 6,579,230; 5,704,895; 5,250,020; 5,048,510 and 4,875,472.

A fibrosis-inhibiting drug combination (or individual component(s) thereof) may be incorporated into, onto or near the device. Penile implants, which may be combined with drug combinations (or individual components thereof)according to the present invention, include commercially available products, such as, for example, the TITAN Inflatable Penile Prosthesis from Mentor Corporation (Santa Barbara, Calif.) and the AMS penile prosthesis product line including the AMS 700 CX CXM, AMS AMBICOR, and AMS Malleable 600M Penile Prostheses from American Medical Systems, Inc. (Minnetonka, Minn.),

In one aspect, the present invention provides penile implant devices that include an anti-scarring drug combination (or individual component(s) thereof) or a composition that includes an anti-scarring drug combination (or individual component(s) thereof). Numerous polymeric and non-polymeric delivery systems for use in penile implants have been described above. Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) or directly to the device surface or to a linker (small molecule or polymer) that is coated or attached to the device surface. The coatings can be applied to different portions of the device. For example, the coating can be (a) a coating applied to the external surface of the portion of the penile implant that is implanted into the penis; (b) a coating applied to the external surfaces of the portions of the penile implant that are implanted in the scrotum, or (c) a coating applied to all or parts of the surfaces of the entire device.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting agent is incorporated into the final device.

In addition to incorporation of a fibrosis-inhibiting drug combination (or individual component(s) thereof) into or onto the device, another biologically active drug combination (or individual component(s) thereof) can be incorporated into or onto the device, for example an anti-inflammatory (e.g., dexamethazone or aspirin).

In another aspect, the device may further comprise an antibiotic or a combination of an antibiotic and an anti-inflammatory agent in order to combat infection associated with implantation of penile implants.

The placement of penile implants can be complicated by infection (usually in the first 6 months after surgery) with Coagulase Negative Staphylococci (including Staphylococcus epidermidis), Staphylococcus aureus, Pseudomonas aeruginosa, Enterococci, Serratia and Candida. Infection is characterized by fever, erythema, induration and purulent drainage from the operative site. The usual route of infection is through the incision at the time of surgery and up to 3% of penile implants become infected despite the best sterile surgical technique. To help combat this, intraoperative irrigation with antibiotic solutions is often employed.

Drug-coating of, or drug incorporation into, the penile implant can allow bacteriocidal drug levels to be achieved locally, thus reducing the incidence of bacterial colonization (and subsequent development of local infection and device failure), while producing negligible systemic exposure to the drugs.

Representative examples of antibiotics include amoxicillin, trimethoprim-sulfamethoxazole, azithromycin, clarithromycin, amoxicillin-clavulanate, cefprozil, cefuroxime, cefpodoxime, or cefdinir).

Other examples of anti-infective compounds include doxorubicin, mitoxantrone, 5-fluorouracil and etoposide.

Utilizing the fluoropyrimidine, 5-fluorouracil, as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.1 μg-1 mg per mm² of surface area. In a particularly preferred embodiment, 5-fluorouracil should be applied to the implant surface at a dose of 1.0 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

Anti-inflammatory and anti-infective agents may be formulated, for example, into a coating applied to the surface of the penile implant. The drug(s) can be applied in several manners: (a) as a coating applied to the external surface of the penile implant; and/or (b) incorporated into the polymers which comprise the penile implant.

According to the present invention, any fibrosis-inhibiting drug combination (or individual component(s) thereof) described above can be utilized in the practice of this embodiment. Within one embodiment of the invention, penile implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As penile implant devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in penile implants include the following: amoxapine and prednisolone, paroxetine and prednisolone, dipyridamole and prednisolone, dexamethasone and econazole, diflorasone and alprostadil, dipyridamole and amoxapine, dipyridarnole and ibudilast, nortriptyline and loratadine (or desloratadine), albendazole and pentamidine, itraconazole and lovastatin, and terbinafine and manganese sulfate.

Regardless of the method of application of the drug to the penile implant, the exemplary anti-fibrosing drug combination (or individual component(s) thereof) should be administered under the following dosing guidelines. The total amount (dose) of anti-scarring agent(s) in or on the penile implant may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μ/mm².

Provided below are exemplary dosage ranges for various anti-scarring drug combinations (or individual components thereof) that can be used in conjunction with penile implant devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Endotracheal and Tracheostomy Tubes

In one aspect, the present invention provides for the combination of an anti-scarring drug combination (or individual component(s) thereof) and endotracheal and tracheostomy tube devices. Association of an anti-scarring drug combination (or individual component(s) thereof) with an endotracheal or a tracheostomy tube (e.g., chest tube) may be used to prevent stenosis of the artificial airway.

Endotracheal tubes and tracheostomy tubes are used to maintain the airway when ventilatory assistance is required. Endotracheal tubes tend to be used to establish an airway in the acute setting, while tracheostomy tubes are used when prolonged ventilation is required or when there is a fixed obstruction in the upper airway.

In one aspect, endotracheal tubes may be used to provide a mechanical air passageway, which may be required for ventilation of the lungs during injury or surgery. Endotracheal tubes may have a single lumen or double lumen, and may have a flange or balloon for engaging its position within the trachea. For example, the endotracheal tube may be composed of an inner and outer flexible tube having a radially extending flange that prevents advancement beyond the larynx. See, e.g., U.S. Pat. No. 5,259,371. The endotracheal tube may have a double lumen which is removably affixed whereby the first tubular lumen may be removed from the airway while the second tubular lumen remains intact. See, e.g., U.S. Pat. No. 6,443,156. The endotracheal tube may have a tracheal portion and a bronchial portion attached at an angle that forms a single lumen, whereby when a balloon that is positioned within the tube is inflated, it blocks the flow of gas through the bronchial portion. See, e.g., U.S. Pat. No. 6,609,521. The endotracheal tube may be composed of two cylindrical portions of different diameters which are connected by a non-circularly shaped tapered portion to complement the glottis which has a plurality of sealing gills that are thin and pliable that extends from the tapered portion. See, e.g., U.S. Pat. No. 5,429,127. The endotracheal tube may be composed of a tubular portion with a visual indicator to provide guidance of the rotational orientation of the beveled tip at the distal end as it is advanced along the airway. See, e.g., U.S. Pat. No. 6,568,393. The endotracheal tube may be composed of a light reflective coated bore to enhance image transmission and a flexible plurality of passages, one adapted to receive a fiber optic bundle, another connected to an inflatable cuff, and another adapted to receive a malleable stylette to aid in insertion and removal. See, e.g., U.S. Pat. No. 6,629,924. The endotracheal tube may be composed of a hollow, flexible, cylindrical tube having an annular flange at its tip and a connector with an annular internal ridge that is concentrically mounted upon the outer proximal surface of the tube portion. See, e.g., U.S. Pat. No. 5,251,617. The endotracheal tube may be composed of a main tube with an inflatable cuff for sealing, which has a double lumen for irrigation and suction for removal of secretions that may pool in the trachea. See, e.g., U.S. Pat. No. 5,143,062. Other endotracheal tubes are described in, e.g., U.S. Pat. Nos. 6,321,749; 5,765,559; 5,353,787; 5,291,882 and 4,977,894.

Tracheostomy tubes can be used to provide a bypass supply of air when the throat is obstructed. Tracheostomy tubes are used with an obturator for percutaneous insertion into a trachea through a stoma in the neck between adjacent cartilages to assist breathing. For example, the tracheostomy tube may be a tubular cannula formed of soft flexible plastic material which has a tapered distal end that is beveled, narrow, angled and curved downwardly for positioning within the trachea. See, e.g., U.S. Pat. No. 5,058,580. The tracheostomy tube may be composed of a tube with a removable fitting mounted on the exposed end which may be sealed to the tube. See, e.g., U.S. Pat. No. 5,606,966. The tracheostomy tube may be composed of an arcuate cannula with a flange that extends laterally outward and a rotatable tubular elbow that has a fluid connection with the cannula. See, e.g., U.S. Pat. Nos. 5,259,376 and 5,054,482. The tracheostomy tube may be composed of two airways with a pneumatic vibrator that generates sonic vibrations to permit audible speech. See, e.g., U.S. Pat. No. 4,773,412. The tracheostomy tube may be composed of an inner cannula removably received within an outer cannula with a sealing cuff between the outer cannula and the trachea to substantially prevent air from escaping from the trachea and to allow phonation through a secondary passageway formed between the inner and outer cannula. See, e.g., U.S. Pat. No. 4,573,460. The tracheostomy tube may be composed of a first port for orienting outside the neck of the wearer, a second port for orienting within the trachea, and a third connecting port to provide and control gas flow via a valve. See, e.g., U.S. Pat. No. 5,957,978. The tracheostomy tube may be composed of a hollow tube, an inflatable balloon having orthogonal projections, and a flange that provides an anchor external to the throat. See, e.g., U.S. Pat. No. 6,612,305. The tracheostomy tube may be composed of a highly flexible material having wire reinforcement and a neck plate with a collar portion that may slide along the tube. See, e.g., U.S. Pat. No. 5,443,064. Other tracheostomy tubes are described in, e.g., U.S. Pat. Nos. 6,662,804; 6,135,110 and 5,983,895.

Endotracheal tubes, which may be combined with one or more anti-fibrosis drug combinations (or individual components thereof) according to the present invention, include commercially available products, such as the HI-LO Tracheal Tubes, LASER-FLEX Tracheal Tubes, and ENDOTROL Tracheal Tubes from Nellcor Puritan Bennett Inc. (Pleasanton, Calif.), the SHERIDAN Endotracheal Tubes from Hudson RCI (Temecula, Calif.), and the BARD Endotracheal Tube, Cuffed from C.R. Bard, Inc. (Murray Hill, N.J.).

Tracheostomy tubes, which may be combined with one or more anti-fibrosis drug combinations (or individual components thereof) according to the present invention, include commercially available products, such as the SHILEY TRACHEOSOFT XLT Tracheostomy Tubes, PHONATE Speaking Valves, and Reusable Cannula Cuffless Tracheostomy Tubes from Nellcor Puritan Bennett Inc. (Pleasanton, Calif.), the PER-FIT Percutaneous Dilational Tracheostomy Kits, PORTEX BLUE LINE Cuffed Tracheostomy Tubes, and BIVONA Uncuffed Tracheostomy Tubes from Portex, Inc. (Keene, N.H.), and the CRYSTALCLEAR Tracheostomy Tubes from Rusch (Germany).

In one aspect, the present invention provides endotracheal and tracheostomy tube devices that include an anti-scarring drug combination (or individual component(s) thereof) or a composition that includes an anti-scarring drug combination (or individual component(s) thereof). Numerous polymeric and non-polymeric delivery systems for use in endotracheal and tracheostomy devices have been described above. Methods for incorporating the fibrosis-inhibiting agent into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the internal (luminal) surface of the endotracheal tube or tracheostomy tube; (b) as a coating applied to the external surface of the endotracheal tube or tracheostomy tube; or (c) as a coating applied to all or parts of both surfaces.

The fibrosis-inhibiting drug combination (or individual component(s) thereof) can be mixed with the materials that are used to make the device such that the fibrosis-inhibiting drug combination (or individual component(s)) is incorporated into the final device.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting drug combination (or individual component(s) thereof) described above can be utilized in the practice of this embodiment. Within one embodiment of the invention, endotracheal and tracheostomy devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As endotracheal and tracheostomy tube devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in endotracheal and tracheostomy tube devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the device, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components)thereof) that can be used in conjunction with endotracheal and tracheostomy tube devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Peritoneal Dialysis Catheters

In one aspect, the present invention provides for the combination of an anti-scarring drug combination (or individual component(s) thereof) and a peritoneal dialysis catheter or a peritoneal implant for drug delivery.

Peritoneal catheters may be used for peritoneal dialysis. Peritoneal dialysis is a form of dialysis in which the blood is not removed from the body but instead, cleansing fluid is put into the abdominal cavity where the body's peritoneum acts as the dialysis membrane. The dialysate equilibrates with plasma for several hours and then the equilibrated dialysate is drained with the associated toxins. The peritoneal catheter is surgically placed into the peritoneal cavity in order to drain dialysate into and out of the peritoneal cavity.

Peritoneal dialysis catheters are typically double-cuffed and tunnelled catheters that provide access to the peritoneum. The most common peritoneal dialysis catheter designs are the Tenckhoff catheter, the Swan Neck Missouri catheter and the Toronto Western catheter. In peritoneal dialysis, the peritoneum acts as a semipermeable membrane across which solutes can be exchanged down a concentration gradient. Continuous peritoneal access catheters are permanently implanted for those that require repeated access to the peritoneum. Implanted peritoneal catheters may be used for peritoneal dialysis or for a means of delivering drug to the peritoneum. These catheters may be composed of synthetic materials, such as silicone, rubber, polyurethane or other polymers that provide flexibility. They may be designed to be configured as a straight tube or may be bent and molded into a variety of shapes to provide different configurations, including helices and coils. The peritoneal catheters may be composed of one continuous element or may be sectioned into parts to provide flanges, cuffs, beads or discs at one of the ends to fix the catheter in position.

For example, the peritoneal catheter may be a resilient, foldable, T-shaped housing chamber with access ports that have elongated, flexible, fluid channels that gather or distribute a liquid such as dialysis fluid. See, e.g., U.S. Pat. No. 5,322,519. The peritoneal catheter may be composed of two linearly mated inflow and outflow conduits contoured as a circular cross-section, which join fluted fluid transport branches. See, e.g., U.S. Pat. No. 6,659,134. The peritoneal catheter may be composed of a ductwork of multiple tubes with fluid holes enclosed within a fluid permeable envelope structure that has slits to allow fluid flow but not tissue adherence. See, e.g., U.S. Pat. No. 5,254,084.

The peritoneal catheter may have a one-half helical turn to provide a radial flow and be composed of a plurality of ingress and egress ports positioned about its circumference and length, and have a coating of ultra low temperature isotropic carbon on the intra-abdominal section. See, e.g., U.S. Pat. No. 5,098,413. The peritoneal catheter may be an elongated flexible tube with one end connected to a pair of spaced apart sheets that extends exteriorly into the body cavity with at least one cuff for preventing catheter infections. See, e.g., U.S. Pat. No. 4,368,737. The peritoneal catheter may be composed of two sections which includes a retainer section that permanently ingrows into the abdominal wall and an elongated flexible tube section for delivering and withdrawing dialysate. See, e.g., U.S. Pat. No. 4,278,092. The peritoneal catheter may be flexible tube having a natural bent segment between the proximal and distal ends which includes a flange extending circumferentially at a nonperpendicular angle relative to the axis of the catheter tube. See, e.g., U.S. Pat. No. 4,687,471. The peritoneal catheter may be a percutaneous access device composed of a cylindrical neck portion for skin protrusion, an annular skirt portion for anchoring into the dermis/subcutaneous tissue, and a catheter tube that may be threaded through the neck and skirt portions that has flexible bellows which can form a 90 degree angle. See, e.g., U.S. Pat. No. 4,886,502. The peritoneal catheter may be a flexible, elongated tube with perforations in the wall to pass fluid with a means for urging the central portion of the tube into a tightly wound cylindrical helix configuration. See, e.g., U.S. Pat. No. 4,681,570. Other examples of peritoneal catheters used for dialysis are described in, e.g., U.S. Pat. Nos. 6,290,669; 5,752,939 and 5,171,227.

In another aspect, the peritoneal catheter may be used to administer drugs to the peritoneum. For example, the peritoneal catheter may be a subcutaneous injection catheter apparatus having a receiving chamber with a penetrable membrane to accommodate an injection needle, which may be interconnected to the peritoneal cavity by a hollow stem. See, e.g., U.S. Pat. No. 4,400,169. The peritoneal catheter may be composed of a porous outer casing defining an inner space with an inlet and outlet catheter of non-porous material which are in communication with an opening of the outer casing to form two passageways. See, e.g., U.S. Pat. No. 5,100,392.

Long-term use of peritoneal catheters may lead to infections or blockage of the catheter due to fibrin formation. Synthetic peritoneal catheters and delivery devices that include a fibrosis-inhibiting drug combination (or individual component(s) thereof) or a composition comprising a fibrosis-inhibiting drug combination (or individual component(s) thereof) are capable of preventing stenosis.

Peritoneal catheters, which may be combined with one or more agents according to the present invention, include commercially available products. For example, Cook Critical Care (Bloomington, Ind.) sells the Spiral Chronic Peritoneal Dialysis Catheters and Tenckhoff Chronic Peritoneal Dialysis Catheters. Bard Access Systems (Salt Lake City, Utah) sells the Tenckhoff and HEMOSPLIT Peritoneal Dialysis Catheters. CardioMed Supplies, Inc (ON, Canada) sells the Single Cuff and Double Cuff Straight Peritoneal Dialysis Catheters, as well as the Single Cuff and Double Cuff Coiled Peritoneal Dialysis Catheters. Other companies that sell Single and Double Cuff, Straight and Coiled Tenckhoff catheters and other types of peritoneal catheters include Baxter International, Inc. (Deerfield, Ill.), Fresenius Medical Care (Lexington, Mass.) and Gambro AB (Sweden).

In one aspect, the present invention provides peritoneal access catheters that include an anti-scarring drug combination (or individual component(s) thereof) or a composition that includes an anti-scarring drug combination (or individual component(s) thereof). Numerous polymeric and non-polymeric delivery systems for use in peritoneal dialysis implants and catheters have been described above.

Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the external surface of the graft; (b) as a coating applied to the internal (luminal) surface of the graft; (c) as a coating applied to the superficial cuff; (d) as a coating applied to the deep cuff; or (e) as a coating applied to a combination of these surfaces.

The fibrosis-inhibiting drug combination (or individual component(s) thereof) can be mixed with the materials that are used to make the device such that the fibrosis-inhibiting drug combination (or individual component(s) thereof) is incorporated into the final device.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic including sulfonamides, penicillins, cephalosporins, aminoglycosides (e.g., amoxicillin, trimethoprim-sulfamethoxazole, azithromycin, clarithromycin, bacitracin, polymixin, chloramphenicol, erythromycin, clindomycin, amoxicillin-clavulanate, cefprozil, cefuroxime, cefpodoxime, or cefdinir).

According to the present invention, any fibrosis-inhibiting drug combination (or individual component(s) thereof) described above can be utilized in the practice of this embodiment. Within one embodiment of the invention, peritoneal dialysis implants and catheters may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As peritoneal access catheters devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Preferred fibrosis-inhibiting drug combination (or individual component(s) thereof) for use in peritoneal access catheters and implants include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the device, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agents are applied may be in the range of about 0.01 μg/mm²-1 μg/mm², or 1 μg/mm² -10 μg/mm, or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with peritoneal access catheter devices and implants in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Central Nervous System Shunts and Pressure Monitoring Devices

In one aspect, the present invention provides for the combination of an anti-scarring drug combination (or individual component(s) thereof) and a central nervous system (CNS) device, such as a CNS shunt or a pressure monitoring device. CNS devices that comprise an anti-scarring drug combination (or individual component(s) thereof) are capable of preventing stenosis and obstruction of the device leading to hydrocephalus and increased intercranial pressure.

Hydocephalus, or accumulation of cerebrospinal fluid (CSF) in the brain, is a frequently encountered neurosurgical condition arising from congenital malformations, infection, hemmorrhage, or malignancy. The incompressible fluid exerts pressure on the brain leading to brain damage or even death if untreated. CNS shunts are conduits placed in the ventricles of the brain to divert the flow of CSF from the brain to other body compartments and relieve the fluid pressure. Ventricular CSF is diverted via a prosthetic shunt to a number of drainage locations including the pleura (ventriculopleural shunt), jugular vein, vena cava (VA shunt), gallbladder and peritoneum (VP shunt; most common).

Representative examples of CNS devices include, e.g., CNS shunts, such as ventriculopleural shunts, jugular vein and vena cava (VA) shunts, and ventriculoperitoneal shunt (VP shunt), such as gallbladder and peritoneum shunts; External Ventricular Drainage (EVD) devices; and Intracranial Pressure (ICP) Monitoring Devices. Other CNS devices include, e.g., dural patches and implants to prevent epidural fibrosis post-laminectomy; and devices for continuous subarachnoid infusions.

In one aspect, the CNS device may be a drainage shunt used to drain fluids in the brain. For example, the CNS device may be a cerebrospinal shunt composed of two tubes whereby an inner tube supplies the fluid from the brain ventricles to the peritoneum region and an outer tube is arranged to exert pressure on the inner tube as the volume of fluid builds in the outer tube. See, e.g., U.S. Pat. No. 5,405,316. The CNS device may be a ventricular drainage system adapted for connection to a ventricular drainage catheter for receiving cerebrospinal fluid and having a valve for controlling fluid flow therethrough. See, e.g., U.S. Pat. No. 5,772,625. The CNS device may be a brain ventricular shunt system composed of a brain check valve for preventing cerebrospinal fluid backflow and a flow-rate switching mechanism to provide flow of cerebrospinal fluid from the brain ventricle catheter to the peritoneum or auricle catheter. See, e.g., U.S. Pat. No. 4,781,673. The CNS device may be shunt member with a flow restricting passage that is connected to catheters to provide cerebrospinal fluid drainage from the brain ventricle to the sinus sagittalis. See, e.g., U.S. Pat. No. 6,283,934. The CNS device may be a ventricular end of a ventriculo-cardiac shunt that has a closed distal end with lateral passageways adjacent thereto which are porous and expansible for providing an umbrella-like liner to allow passage of fluid while preventing obstruction. See, e.g., U.S. Pat. No. 3,690,323. The CNS device may be a hydrocephalus valve composed of a chamber with an inlet and outlet valve for routing cerebrospinal fluid away from the brain at a controlled pressure. See, e.g., U.S. Pat. No. 5,069,663. The CNS device may be a hydrocephalus device composed of an external, flexible shell forming a fluid reservoir and housing a non-obstructive, self-regulating valve having a folded membrane which forms a slit-like opening, which has inlet and outlet tubes. See, e.g., U.S. Pat. No. 5,728,061. The CNS device may be a cerebral spinal fluid draining shunt composed of an implantable master control unit that interconnects a cerebral spinal space catheter with a catheter that drains the fluid into a body cavity. See, e.g., U.S. Pat. No. 6,585,677. The CNS device may be a cerebrospinal fluid shunt composed of a ventricular catheter connected to a flexible drainage tube which has an exterior flexible tubular cover from which the drainage tube may be drawn. See, e.g., U.S. Pat. No. 4,950,232. The CNS device may be an intracranial shunting tube composed of a thin film that extends radially and outwardly from the open end of a ventricular tube which has a plurality of side holes to bypass ventricular cerebrospinal fluid to the subdural space on the surface of the brain. See, e.g., U.S. Pat. No. 5,000,731. Other CNS shunts are described in, e.g., U.S. Pat. Nos. 6,575,928; 5,437,626 and 4,631,051.

In another aspect, the CNS device may be a pressure monitoring device. For example, the pressure monitoring device may be an intracranial pressure sensor which is mounted within the skull of a body at the situs where the pressure is to be monitored and a means of transmitting the pressure externally from the skull. See, e.g., U.S. Pat. No. 4,003,141. The pressure monitoring device may be a telemetric differential pressure sensitive device composed of a thin, planar, closed, conductive loop which moves with a flexible diaphragm upon changes in the difference of two bodily pressures on its opposite sides. See, e.g., U.S. Pat. No. 4,593,703. The pressure monitoring device may be composed of a radio-opaque liquid contained within a resiliently compressible vessel of a silastic material in which the volume of liquid is variable as a function of the pressure or force applied to the vessel. See, e.g., U.S. Pat. No. 3,877,137. The pressure monitoring device may be a probe composed of a threaded shaft having a lumen and an engaging lock nut, which is inserted through an opening in the scalp and into the subarachnoid space. See, e.g., U.S. Pat. No. 4,600,013. The pressure monitoring device may be composed of an external transceiver unit and an implantable cavity resonator unit having a dielectric-filled cavity with a predetermined resonance frequency for high frequency electromagnetic waves. See, e.g., U.S. Pat. No. 5,873,840. The pressure monitoring device may be an implantable sensor that detects a physiological parameter (e.g., cerebral spinal fluid flow) and then generates, processes, and transmits the signal to an external receiver. See, e.g., U.S. Pat. No. 6,533,733. Other CNS pressure monitoring devices are described in, e.g., U.S. Pat. Nos. 6,248,080 and 6,210,346.

CNS shunts, which may be combined with one or more agents according to the present invention, include commercially available products, such as the Codman HAKIM Programmable Valves from Codman & Shurtleff, Inc. (Raynham, Mass.), a Johnson & Johnson Company. Other examples include the Integra Neuro Sciences (Plainsboro, N.J.) HEYER-SCHULTE Neurosurgical Shunts, HERMETIC CSF Drainage Systems, and OSV II SMART VALVE Systems and the Medtronic, Inc. (Minneapolis, Minn.) Shunt Assemblies, including the STRATA, DELTA, CSF-Snap and CSF-Flow Control Shunt Assemblies.

Pressure Monitoring CNS devices, which may be combined with one or more agents according to the present invention, include commercially available products such as the VENTRIX Pressure Monitoring Kits and CAMINO Micro Ventricular Bolt ICP Monitoring Catheters from Integra Neuro Sciences (Plainsboro, N.J.).

In one aspect, the present invention provides CNS devices that include an anti-scarring drug combination (or individual component(s) thereof) or a composition that includes an anti-scarring drug combination (or individual component(s) thereof). Numerous polymeric and non-polymeric delivery systems for use in CNS devices have been described above. Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the external surface of the shunt; (b) as a coating applied to the internal (luminal) surface of the shunt; or (c) as a coating applied to all or parts of both surfaces.

The fibrosis-inhibiting drug combination (or individual component(s) thereof) can be mixed with the materials that are used to make the device such that the fibrosis-inhibiting drug combination (or individual component(s) thereof) is incorporated into the final device.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting drug combination (or individual component(s) thereof) described above can be utilized in the practice of this embodiment. Within one embodiment of the invention, CNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As CNS devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Several examples of fibrosis-inhibiting drug combinations (or individual components thereof) for use in CNS devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Regardless of the method of application of the drug to the device, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with CNS devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Inferior Vena Cava Filters

In one aspect, the present invention provides for the combination of an anti-scarring drug combination (or individual component(s) thereof) and an inferior vena cava filter device. The term inferior vena cava filters are devices that are intended to capture emboli and prevent them from migrating through the blood stream. Examples of inferior vena cava filters include, without limitation, vascular filters, blood filters, implantable blood filters, caval filters, vena cava filters, vena cava filtering devices, thrombosis filters, thrombus filters, antimigration filters, filtering devices, percutaneous filter systems, intravascular traps, intravascular filters, clot filters, vein filters and body vessel filters.

Inferior vena cava filters catch blood clots to prevent them from traveling to other parts of the body to form an embolus. It may be life threatening if plaques or blood clots migrate through the blood stream and travel to the lungs and cause a pulmonary embolism. To prevent such an occurrence, inferior vena cava filters are placed in the large veins of the body to prevent pulmonary emboli in patients with (or at risk of developing) deep vein thrombosis. Most often these filters are composed of synthetic polymers or metals. These filters may be a variety of configurations, including but not limited to, baskets, cones, umbrellas or loops. The shape of the filter must provide adequate trapping ability while allowing sufficient blood flow. Along with the functional shape, filters may also have other design features including peripheral loops for alignment or anchoring features to prevent migration (e.g., ridges, struts or sharp points). Where the filter comes into contact with the vessel wall for anchoring, a fibrotic response may occur. This fibrotic response can result in difficulties in removal of the filter. This is a particular problem for filters that are to be kept in place for a relatively short period of time. Incorporation of a fibrosis-inhibiting agent into or onto the filter may reduce or prevent stenosis or obstruction of the device via a fibroproliferative response.

In one aspect, inferior vena cava filters may be designed in a variety of configurations. For example, the inferior vena cava filter may be composed of a plurality of intraluminal filter elements held by a retainer in a filter configuration that may be released to an open, stent-like configuration. See, e.g., U.S. Pat. No. 6,267,776. The inferior vena cava filter may be composed of an embolus capturing portion having a plurality of elongated filter wires diverging in a helical arrangement to form a conical surface and an anchoring portion that has a plurality of struts. See, e.g., U.S. Pat. No. 6,391,045. The inferior vena cava filter may be composed of a textured echogenic feature so the filter position may be determined by sonographic visualization. See, e.g., U.S. Pat. No. 6,436,120. The inferior vena cava filter may be composed of a plurality of core wire struts that are anchored to radiate outwardly which are interconnected by compression material to form a filter basket. See, e.g., U.S. Pat. No. 5,370,657. The inferior vena cava filter may be composed of an apical head with a plurality of divergent legs in a conical shaped geometry which have a hook and pad for securing to the vessel. See, e.g., U.S. Pat. No. 5,059,205. The inferior vena cava filter may be composed of a filtering device made of shape memory/superelastic material formed at the distal end of a deployment/retrieval wire section for minimally invasive positioning. See, e.g., U.S. Pat. No. 5,893,869. The inferior vena cava filter may be composed of a plurality of intraluminal elements joined by a retainer, whereby upon release of the retainer, the intraluminal filter elements convert to an open configuration in the blood vessel. See, e.g., U.S. Pat. Nos. 6,517,559 and 6,267,776. The inferior vena cava filter may be composed of an outer catheter and an inner catheter having a collapsible mesh-like filter basket at the distal end made of spring wires or plastic monofilaments. See, e.g., U.S. Pat. No. 5,549,626. The inferior vena cava filter may be composed of a plurality of radiating struts that attach at a body element and has a two layer surface treatment to provide endothelial cell growth and anti-proliferative properties. See, e.g., U.S. Pat. No. 6,273,901. The inferior vena cava filter may be composed of a metal fabric that is configured as a particle-trapping screen that may be slideable along a guidewire. See, e.g., U.S. Pat. No. 6,605,102. The inferior vena cava filter may be non-permanent with a single high memory coiled wire having a cylindrical and a conical segment. See, e.g., U.S. Pat. No. 6,059,825. Other inferior vena cava filters are described in, e.g., U.S. Pat. Nos. 6,623,506; 6,391,044; 6,231,589; 5,984,947; 5,695,518 and 4,817,600.

Vena cava filters, which may be combined with one or more anti-scarring drug combinations (or individual components thereof) according to the present invention, include commercially available products. Examples of vena cava filters that can benefit from the incorporation of a fibrosis-inhibiting agent include, without limitation, the GÜNTHER TULIP Vena Cava FILTER and the GIANTURCO-ROEHM BIRD'S NEST Filter which are sold by Cook, Inc. (Bloomington, Ind.). C.R. Bard (Murray Hill, N.J.) sells the SIMON-NITINOL FILTER and RECOVERY Filter. Cordis Endovascular which is a subsidiary of Cordis Corporation (Miami Lakes, Fla.) sells the TRAPEASE Permanent Vena Cava Filter. B. Braun Medical Inc. (Bethlehem, Pa.) sells the VENA TECH LP Vena Cava Filter and VENA TECH-LGM Vena Cava Filter. Boston Scientific Corporation (Natick, Mass.) sells the Over-the-Wire GREENFIELD Vena Cava Filter.

In one aspect, the present invention provides inferior vena cava filter devices that include an anti-scarring drug combination (or individual component(s) thereof) or a composition that includes an anti-scarring drug combination (or individual component(s) thereof). Numerous polymeric and non-polymeric delivery systems for use in inferior vena cava filters have been described above. These compositions can further comprise a fibrosis-inhibiting drug combination (or individual component(s) thereof) such that the overgrowth of granulation tissue is inhibited or reduced.

Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the entire leg of the filter; (b) as a coating applied to the tips of the filter that come into contact with the blood vessel; and/or (c) as a coating applied to all or parts of the entire filter device.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole), and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting drug combination (or individual component(s) thereof) described above can be utilized in the practice of this embodiment. Within one embodiment of the invention, vena cava filters (e.g., inferior vena cava filters) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Several examples of fibrosis-inhibiting drug combinations (or individual components thereof) for use in vena cava filter devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

As vena cava filter devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Regardless of the method of application of the drug to the device, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof)that can be used in conjunction with vena cava devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Gastrointestinal Devices

In one aspect, the present invention provides for the combination of an anti-scarring drug combination (or individual component(s) thereof) and a gastrointestinal (GI) device. There are many gastrointestinal tube devices that are used for feeding applications and for drainage applications. The functioning of these tubes can be compromised if there is an excessive fibroproliferative response to these devices. The incorporation of a fibrosis-inhibiting drug combination (or individual component(s) thereof) into or onto the device can modulate this fibroproliferative response (e.g., to prevent stenosis and/or obstruction of the device) thereby maintaining performance of the device.

A variety of GI tubes for drainage or feeding can be combined with a fibrosis-inhibiting drug combination (or individual component(s) thereof) to prevent stenosis and/or obstruction of the device. These devices may include, without limitation, GI tubes for drainage or feeding, portosystemic shunts, shunts for ascites, nasogastric or nasoenteral tubes, gastrostomy or percutaneous feeding tubes, jejunostomy endoscopic tubes, colostomy devices, drainage tubes, biliary T-tubes, biopsy forceps, biliary stone removal devices, endoscopic retrograde cholangiopancretography (ERCP) devices, dilation balloons, enteral feeding devices, stents, low profile devices, virtual colonoscopy (VC) devices, capsule endoscopes, and retrieval devices.

GI devices may be composed of synthetic materials, including, without limitation, stainless steel, metals, nitinol, glass, resins or polymers. In one aspect, the GI device may be an instrument used to examine or provide access to the interior of the gastrointestinal tract. This may include optical imaging in the form of still imaging or videoing for diagnosing purposes. Procedures that use these devices include, without limitation, enteroscopy, colonoscopy or esophagogastroduodenoscopy, where an endoscope enters the esophagus or anal canal to assess portions of the GI tract. For example, the GI device may be an endoscope having a tubular shaft for receiving a viewing lens and a treatment instrument. See, e.g., U.S. Pat. No. 5,421,323. The GI device may be a multi-lumen endoscopic catheter that may be inserted through an endoscope for the practice of endoscopic retrograde cholangiopancreatography, whereby the first lumen has a wire threaded through it, the second lumen provides a conduit to infuse a radio-opaque contrast medium to identify obstructions, and the third lumen provides a conduit to dilate a balloon. See, e.g., U.S. Pat. Nos. 5,788,681 and 5,843,028. The GI device may be a video endoscope system composed of a swallowable capsule, a transmitter and a reception system. See, e.g., U.S. Pat. No. 5,604,531. The GI device may be an endoscope composed of an encapsulated ultrasonic transducer capsule having a self-contained electromechanical sector scanner, which may be used for transesophageal echocardiography. See, e.g., U.S. Pat. Nos. 4,977,898 and 4,834,102. The GI device may be a sterilizable endoscope having an image sensor mounted on a cylindrical capsule and a separable disposable channel. See, e.g., U.S. Pat. No. 5,643,175. The GI device may be a body canal intrusion instrument that may be composed of a bi-directional surface friction for engaging tissue during navigation to decrease the risk of puncture and time associated with the insertion of catheters, guidewires and endoscopes through body cavities and canals. See, e.g., U.S. Pat. No. 6,589,213. The GI device may be a colonic access device composed of flexible tubing with a tether for releasing from a colonoscope, which may be placed in the colon for up to several days to monitor and treat colorectal diseases. See, e.g., U.S. Pat. No. 6,149,581. The GI device may be adapted for the bile or pancreatic duct by being composed of a mother endoscope that is inserted into the duodenum and a daughter endoscope that is inserted via papilla through a forceps channel. See, e.g., U.S. Pat. No. 4,979,496.

In another aspect, the GI device may be used as a conduit for long-term tube feeding. These GI devices may include, without limitation, percutaneous feeding tubes, enteral feeding devices/catheters, gastrostomy feeding tubes, low profile devices, and nasogastric tubes. These long-term feeding tubes may be advanced through the GI tract via nasal canal or through the abdominal wall via a gastrostomy. For example, the GI device may be an enteral feeding catheter adapted to serve as a conduit for passage of sustenance through an abdominal wall into the body and having a retainer and retractable locking means. See, e.g., Pat. No. 4,826,481. The. GI device may be an enteral feeding tube having a catheter that allows for easy insertion and removal by having a slim, tapered guide tube and a balloon bolster. See, e.g., U.S. Pat. No. 6,582,395. The GI device may be an enteral feeding device for administering fluids into the stomach, which is composed of a female connector, flexible feeding tube, fluid discharge tube, and probe, which are connected to the male end of the guide wire. See, e.g., U.S. Pat. No. 5,242,429. The GI device may be a hollow, cylindrical elongated body with a spring-biased valve, which is maintained through a surgical opening in the stomach wall by an extended concentric flange that facilitates fixation. See, e.g., U.S. Pat. No. 4,344,435. The GI device may be a nasogastric tube having openings along its distal end with a coupled introducer flexible sheath extending longitudinally along the tube. See, e.g., U.S. Pat. No. 5,334,167. Other GI devices used as feeding tubes or related devices are described in, e.g., U.S. Pat. Nos. 6,582,395; 5,989,225; 5,720,734; 5,716,347; 5,503,629; 5,342,321; 4,861,334; 4,758,219 and 4,057,065.

In another aspect, the GI device may be used for irrigation or aspiration of the GI tract. These GI devices may be used, for example, to remove ingested poisons or blood, to treat absorption-related conditions, to decompress the stomach, pre-operatively to ensure portions of the GI tract is empty, post-operatively to remove gas, and to treat diseases such as bowel obstructions or paralytic ileus. For example, the GI tube may be elongated and configured to be inserted in the GI tract having a slidable treatment device for controlling bleeding and a fluid reservoir coupled to the tube. See, e.g., U.S. Pat. No. 5,947,926. The GI tube may be a nasogastric flexible tube with a curved or bent leading end to anatomically conform and facilitate advancement into the esophagus and stomach. See, e.g., U.S. Pat. No. 5,690,620. The GI tube may be a nasogastric elongated tube fixedly bent to extend from the nostril without affixation to avoid pressure necrosis in the nose due to force exertion. See, e.g., U.S. Pat. No. 4,363,323. The GI device may be composed of aspirating, feeding and inflation lumens, which is surgically inserted through the abdominal and gastric wall. See, e.g., U.S. Pat. No. 4,543,089. The GI device may be composed of drain tube and irrigating tube with a cuffed fluid sealing that is used for unidirectional irrigation of the bowels. See, e.g., U.S. Pat. No. 4,637,814. The GI device may be an open-ended, thin-walled, balloon-like tube shaped to extend through at least part of an alimentary canal for the purpose of passing digested food solids and thereby treating absorption-related diseases. See, e.g., U.S. Pat. Nos. 4,315,509 and 4,134,405.

In another aspect, the GI device may be a colostomy device. For example, the colostomy device may be an artificial anus composed of a hollow tubular support with a cylindrical body having a pair of radially-extending flanges to engage the member See, e.g., U.S. Pat. No. 4,781,176. The colostomy device may be composed of internal and external balloons connected by a tube and an annular supporting plate for attachment to the stoma or rectum. See, e.g., U.S. Pat. No. 5,569,216.

In another aspect, the GI device may be a mechanical hemostatic device used to control GI bleeding. Hemostatic devices, which are used to constrict blood flow, may include, without limitation, clamps, clips, staples and sutures. For example, the hemostatic device may be a compression clip composed of an anchor and stem having a transverse hole and a bolster which may be fixed or movable along the stem. See, e.g., U.S. Pat. No. 6,387,114. The hemostatic device may be an endoscopic clip composed of deformable material and a tissue-penetrating pair of hollow jaws. See, e.g., U.S. Pat. No. 5,989,268.

In another aspect, the GI device may be a means to clear blocked GI tracts. For example, the GI device may be a dilation catheter composed of a shroud tube having a strain relief tube extending from within which is used to alter the configuration of a dilation balloon. See, e.g., U.S. Pat. No. 6,537,247.

In another aspect, the GI device may function to deliver drug to the GI tract. For example, the GI device may be orally administered and composed of a two-chambered water-permeable body, in which one chamber has an orifice for expelling a liquid drug when under pressure, and the second chamber contains an electric circuit that generates a gas which compresses the first chamber to expel the drug. See, e.g., U.S. Pat. No. 5,925,030. The GI device may be a collapsible, ellipsoidal gastric anchor with a tether and a long, narrow intestinal payload module, which contains slow release medicaments, bound enzymes or nonpathogenic microorganisms. See, e.g., U.S. Pat. No. 4,878,905. The GI device may be an ingestible device for delivering a substance to a chosen site within the GI tract, which includes a receiver of electromagnetic radiation for powering an openable part of the device for inserting or dispensing the substance. See, e.g., U.S. Pat. No. 6,632,216.

In another aspect, the GI device may be a shunting device used to provide communication between two bodily systems. Shunting devices may be used to treat abnormal conditions, such as bypassing occlusions in a body passageway or transferring unwanted accumulation of fluids from a body cavity to a site where it can be processed by the body. For example, a shunting device may be used to displace peritoneal cavity fluid into the systemic venous circulation as a treatment for ascites. Shunting devices may include, without limitation, portosystemic shunts and peritoneovenous shunts. For example, the shunt may be an implantable pump composed of a cylindrical chamber and port with pumping means for aspirating fluid and expelling fluids. See, e.g., U.S. Pat. No. 4,725,207. The shunt may be an implantable peritoneovenous shunt system composed of a double-chambered ascites collection device, a pump (e.g., magnetically driven or compression driven), and an anti-reflux catheter, that are all connected by flexible tubing. See, e.g., U.S. Pat. No. 4,657,530 and 4,610,658. The shunt may be composed of a peritoneal tube connected to a hollow plastic implanted valve assembly that passes fluid when under pressure to a venous tube. See, e.g., U.S. Pat. No. 5,520,632. The shunt may be a collapsible, shape-memory metal fabric with a plurality of woven metal strands having a central passageway for fluid and delivered in a collapsed state through a body channel to create a portosystemic shunt. See, e.g., U.S. Pat. No. 6,468,303. The GI device may be a laparoscopic tunneling dissector composed of an inflatable balloon and a hollow blunt tipped obturator which is used to tunnel through tissue to provide an anatomic working space for laparoscopic procedures. See, e.g., U.S. Pat. Nos. 5,836,961 and 5,817,123.

GI devices, which may be combined with one or more agents according to the present invention, include commercially available products.

In one aspect, GI devices that are used for feeding purposes may include a variety of devices. For example, gastrostomy tubes such as the DURA-G Polyurethane Gastrostomy Tubes and MAGNA-PORT Gastrostomy Tubes are sold by Ross Products (Columbus, Ohio.), a division of Abbott Laboratories. Moss Tubes, Inc. (West Sand Lake, N.Y.) sells the MOSS G-Tube Percutaneous Endoscopic Gastrostomy Kits. Other enteral feeding tubes include, for example, EASY-FEED Enteral Feeding Sets which are sold by Ross Products (Columbus, Ohio.), a division of Abbott Laboratories. COMPAT Enteral Delivery Systems are sold by Novartis AG (Basel, Switzerland). CORFLO Feeding Tubes are sold by VIASYS Healthcare Medsystems Division (Wheeling, Ill.). ENDOVIVE Enteral Feeding Systems are sold by Boston Scientific Corporation. Nasogastric tubes, such as the Mark IV Nasal (SIL) Tubes are sold by Moss Tubes, Inc. (West Sand Lake, N.Y.). Bard Medical Division (Covington, Ga.) of C.R. Bard, Inc. and Andersen Products Limited (England, United Kingdom) also sells a variety of Nasogastric Feeding Tubes. Low profile devices, such as the Low-Profile Replacement Gastrostomy Devices and the Bard Button Replacement Gastrostomy Devices are sold by Bard Endoscopic Technologies (Billerica, Mass.), a division of C.R. Bard, Inc.

In another aspect, GI devices may include gastrointestinal tubes for irrigation or aspiration, such as the LAVACUATOR Gastro Intestinal Tubes and VENTROL Levine Tubes, which are sold by Nellcor Puritan Bennett Inc. (Pleasanton, Calif.).

In another aspect, GI devices may include those used as portosystemic shunts or other shunting devices, such as the VIATORR TIPS Endoprostheses that are sold by W.L. Gore & Associates, Inc. (Newark, Del.). Denver Ascites Shunts are sold by Denver Biomedical, Inc. (Golden, CO). LEVEEN Shunts are sold by Becton, Dickinson and Company (Franklin Lakes, N.J.).

In another aspect, GI devices may include colostomy devices, such as ASSURA Pouches and COLOPLAST Pouches, which are sold by Coloplast Corporation (Marietta, Ga.). ESTEEM SYNERGY Standard Closed-End Pouches and SUR-FIT NATURA Closed-End Pouches are sold by ConvaTec (Princeton, N.J.), a Bristol-Myers Squibb Company. Cymed Ostomy Company (Berkeley, Calif.) sells the MICROSKIN Colostomy Pouching Systems. KARAYA 5 One-Piece Pouching Systems, CONTOUR I One-Piece Ostomy Pouching Systems, and CENTERPOINTLOCK (CPL) Two-Piece Pouching Systems are sold by Hollister Inc. (Libertyville, Ill.). Bard Medical Division (Covington, Ga.) of C.R. Bard, Inc. also sells a variety of Colostomy Pouches.

In another aspect, GI devices may include dilatation catheters, such as the ELIMINATOR Multi-Stage Balloon Dilators, which are sold by Bard Endoscopic Technologies (Billerica, Mass.), a division of C.R. Bard, Inc. CRE Fixed Wire and Wireguided Balloon Dilators are sold by Boston Scientific Corporation (Natick, Mass.).

In one aspect, the present invention provides GI devices that include an anti-scarring drug combination (or individual component(s) thereof) or a composition that includes an anti-scarring drug combination (or individual component(s) thereof). Numerous polymeric and non-polymeric delivery systems have been described above. These compositions can further comprise one or more fibrosis-inhibiting drug combinations (or individual components thereof) such that the overgrowth of granulation tissue is inhibited or reduced.

Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the external surface of the tube; (b) as a coating applied to the internal (luminal) surface of the tube; (c) as a coating applied to the ends of the tube; and/or (d) as a coating applied to all or parts of both surfaces of the tube.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any anti-scarring drug combination (or individual component(s) thereof) described above can be utilized in the practice of this embodiment. Within one embodiment of the invention, GI devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations (or individual components thereof) for use with GI devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate. As GI devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Regardless of the method of application of the drug to the device, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.0 1 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with GI devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Central Venous Catheters

In one aspect, the present invention provides for the combination of an anti-scarring drug combination (or individual component(s) thereof) and a central venous catheter (CVC) device. For the purposes of this invention, the term “Central Venous Catheters” should be understood to include any catheter or line that is used to deliver fluids to the large (central) veins of the body (e.g., jugular, pulmonary, femoral, iliac, inferior vena cava, superior vena cava, axillary etc.). CVC devices are generally hollow, tubular cannulae that are inserted into body passageways to permit injection or withdrawal of bodily fluids. CVCs may be inserted into a large vein, such as the superior vena cava, with a portion of the catheter disposed within the body and a connection port which extends out of the body for access to the circulatory system. CVCs may be used to administer drugs (e.g., chemotherapy or antibiotic therapy) or intravenous feeding, pressure monitoring or periodic blood sampling.

CVCs may be designed with or without a cuff or flange. Cuffs are used to prevent the catheter from slipping or becoming infected. CVCs may have one lumen or multiple lumens and range in many sizes to adapt to the required needs. They may be composed of synthetic materials, including, but not limited to, polyurethane, polyethylene, silicone, copolymers and other polymeric compositions.

CVCs are typically left in the body for a long period of time and thus, may develop infection or inflammation in response to the catheter. CVC access lumens may be blocked by clotted blood or thrombus formation. Some CVCs may also be available with coatings and treated surfaces to minimize the risk of infection and/or inflammation. The incorporation of a fibrosis-inhibiting agent into or onto the device can modulate an excessive fibroproliferative response to the device, which may prevent stenosis and/or obstruction of the device.

In one aspect, the CVC may be designed for specialized access to the circulatory system for specific conditions/purposes. For example, the CVC may be especially made for hemodialysis use by being elongated with a needle-like, dual lumen that may be used as a conduit for administering drugs or additives into the body through an AV access fistula or graft. See, e.g., U.S. Pat. No. 5,876,366. The CVC may be composed of an indwelling cannula adapted for placement within the superior vena cava having an exit port at the distal end whereby fluid medicament may be delivered to essentially the area of subcutaneous tissue surrounding the cannula. See, e.g., U.S. Pat. No. 5,817,072.

In another aspect, the CVC may be designed to provide multiple conduits for accessing the circulatory system. For example, the CVC may be an elongated, integral flexible catheter tube with a plurality of independent lumens that may be adapted for attachment to a separate fluid conveying device whereby fluids may be separately infused into the vein without becoming mixed, and blood may be withdrawn and venous pressure monitored simultaneously with fluid infusion. See, e.g., U.S. Pat. No. 4,072,146. The CVC may be a multi-lumen catheter composed of a central flexible lumen with a formed fluid passageway and a plurality of collapsible lumens mounted around the periphery of the central lumen also having formed fluid passageways therein. See, e.g., U.S. Pat. No. 4,406,656.

In another aspect, the CVC may have a means for preventing infection as a result of long-term use. For example, the CVC may be composed of polyurethane with a thin hydrophilic layer on the surface loaded with an antibiotic of the ramoplanin group to inhibit bacterial colonization on the catheter after insertion. See, e.g., U.S. Pat. No. 5,752,941. The CVC may be composed of a polymeric material that has an outer surface embedded by atoms of an antimicrobial metal (e.g., silver) that extend in a subsurface stratum to form a nonleaching surface treatment. See, e.g., U.S. Pat. No. 5,520,664.

In another aspect, the CVC may be used with an apparatus that provides a means of controlling the injection or withdrawal of bodily fluids through the CVC. For example, the CVC apparatus may be composed of a syringe body with two barrels that have two separate fluid conduits with independent plungers and a valve body. See, e.g., U.S. Pat. No. 5,411,485. The CVC apparatus may be composed of an upper and lower molded sheets and a plurality of syringe channels and barrels that are individually operated by syringe plungers. See, e.g., U.S. Pat. No. 5,417,667. The CVC apparatus may be an integrally molded base sheet which forms opposed slide valve walls that have a plurality of syringes mounted for fluid communication with the inlet ports. See, e.g., U.S. Pat. No. 5,454,792. The CVC apparatus may be composed with access apparatus to provide easier accessibility by being composed of a connector that is in bi-directional fluid communication between a manifold and a CVC. See, e.g., U.S. Pat. No. 5,308,322. The CVC apparatus may be a valve assembly that is provided for the distal end of a CVC for controlling fluid passage from the catheter to the blood flow passage in which it is inserted. See, e.g., U.S. Pat. No. 5,030,210.

Other examples of central venous catheters include total parenteral nutrition catheters, peripherally inserted central venous catheters, flow-directed balloon-tipped pulmonary artery catheters, long-term central venous access catheters (such as Hickman lines and Broviac catheters). Representative examples of such catheters are described in U.S. Pat. Nos. 3,995,623, 4,072,146 4,096,860, 4,099,528, 4,134,402, 4,180,068, 4,385,631, 4,406,656, 4,568,329, 4,960,409, 5,176,661, 5,916,208.

CVCs, which may be combined with one or more agents according to the present invention, include commercially available products. For example, Bard Access Systems (Salt Lake City, Utah) which is a division of C.R. Bard sells the HICKMAN, BROVIAC and LEONARD Central Venous Catheters which are available with SureCuff tissue in-growth cuff and the VitaCuff Antimicrobial Cuff. Edward Lifesciences (Irvine, Calif.) sells the VANTEX Catheter as well as the PRESEP CENTRAL VENOUS OXIMETRY Catheter. Cook Critical Care (Bloomington, Ind.) sells the SPECTRUM Antibiotic Impregnated Catheters as well as other CVC sets and trays. Arrow International (Reading, Pa.) sells the ARROWGARD BLUE Catheters that have single or multiple lumens.

A variety of central venous catheters are available for use in hemodialysis including, but not restricted to, catheters which are totally implanted such as the Lifesite (Vasca Inc., Tewksbury, Mass.) and the Dialock (Biolink Corp., Middleboro, Mass.). Central venous catheters are prone to infection and embodiments for that purpose are described above.

In one aspect, the present invention provides CVC devices that include an anti-scarring drug combination (or individual component(s) thereof) or a composition that includes an anti-scarring drug combination (or individual component(s) thereof). Numerous polymeric and non-polymeric delivery systems have been described above. These compositions can further comprise fibrosis-inhibiting drug combinations (or individual components thereof) such that the overgrowth of granulation tissue is inhibited or reduced.

Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the external surface of the tube; (b) as a coating applied to the internal (luminal) surface of the tube; (c) as a coating applied to the ends of the tube; and/or (d) as a coating applied to all or parts of both surfaces of the tube.

In addition to coating the device with the fibrosis-inhibiting drug combination (or individual component(s) thereof) composition, the fibrosis-inhibiting drug combination (or individual component(s) thereof) can be mixed with the materials that are used to make the device such that the fibrosis-inhibiting drug combination (or individual component(s) thereof) is incorporated into the final device.

In addition to incorporation of a fibrosis-inhibiting drug combination (or individual component(s) thereof) into or onto the device, another biologically active drug combination (or individual component(s) thereof) can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any anti-scarring drug combination (or individual component(s) thereof) described above can be utilized in the practice of this embodiment. Within one embodiment of the invention, CVC devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations (or individual components thereof) for use in CVC devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate. As CVC devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Regardless of the method of application of the drug to the device, the exemplary anti-fibrosing drug combinations (or individual components thereof) should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with CVC devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Ventricular Assist Devices

In one aspect, the present invention provides for the combination of an anti-scarring drug combination (or individual component(s) thereof) and a ventricular assist device (VAD).

Ventrical assist devices are intended to assist the native heart in pumping blood throughout the body. Examples of VADs and other related devices include, without limitation, left ventricular assist devices, right ventricular assist devices, biventricular assist devices, cardiac assist devices, mechanical assist devices, artificial cardiac assist devices, implantable heart assist systems, implantable ventricular assist devices, heart assist pumps and intra-ventricular cardiac assist devices.

VADs are used to treat heart failure where the heart is incapable of pumping blood throughout the body at the rate needed to maintain adequate blood flow. Heart failure includes, without limitation, acute myocardial infarction, cardiomyopathy, cardiac valvular dysfunction, extensive cardiac surgery and uncontrolled cardiac arrhythmias. VADs assist the failing heart by increasing its pumping ability and allowing the heart to rest to recover its normal pumping function. In general, VADs are typically composed of a blood pump that is attached between the ventricle and aorta, cannulae that connect the pump to the heart, and a drive console that powers and controls the device. The most common VAD that exists is the left VAD because the left ventricle of the heart becomes diseased more often than the right ventricle; however, VADs may be used to pump blood from the left ventricle, right ventricle or both ventricles. VADs may be categorized by the pumping drives, which may function as either pulsatile (e.g., intra-aortic balloon pumps) or continuous, (e.g., reciprocating piston-type pumps or rotary pumps (centrifugal or axial impellers)).

VADs, however, may have medical complications associated with the implantation or prolonged use, such as, infections, septic emboli, hemorrhaging, inflammation as a reaction to tissue damage, and thrombosis induced by coagulation or blood stasis. These complications may obstruct the utility of the VAD and may lead to life threatening events. Incorporation of an anti-scarring agent into a VAD may prevent stenosis and/or obstruction of the device.

In one aspect, the VAD may be a pulsatile pump. These devices may have flexible sacks or diaphragms which are compressed and released to provide pulsatile pumping action. One type of pulsatile pump is the intra-aortic balloon pumps (IABP) which is a pulsatile sack device that may be implemented using minimally invasive procedures and are most functional when the left ventricle is able to eject blood to maintain a systemic arterial pressure. For example, the VAD may be an IABP that is a temporary, removable support within the aortic arch that descends through the aorta which has both a depressurized and pressurized position which is maintained by a pumping and blocking balloon. See, e.g., U.S. Pat. No. 6,228,018. The VAD may be an IABP catheter and a pumping chamber having both a large and small diameter portions that are separated by a flexible diaphragm/membrane. See, e.g., U.S. Pat. No. 5,928,132. The VAD may be a pulsatile pump composed of a cannula with an outer sheath and lumen, intake and outlet valves, fluid reservoir, and hydraulic pump that produces a pulsatile pumping action of blood through the cannula. See, e.g., U.S. Pat. No. 6,007,479.

In another aspect, the VAD may be a continuous pump providing mostly steady flow of blood which may include an imperceptible pulsatile component. Continuous pumps may include reciprocating piston-type pumps, such as pneumatically powered devices or magnetically operated devices, and rotary pumps, such as centrifugal or axial impellors. For example, the VAD may be an implantable apparatus with a stator member and a magnetically suspended rotor member that act as a centrifugal pump where an impeller draws blood from the left ventricle and delivers it to the aorta thereby reducing the left ventricle pressure. See, e.g., U.S. Pat. No. 5,928,131. The VAD may be composed of an implantable reciprocating piston for driving an implanted blood-pumping mechanism which is controlled by external electromagnets. See, e.g., U.S. Pat. No. 5,089,017.

In another aspect, the VAD may be a device for assisting the pumping capacity of one of either the left or right ventricle. For example, the VAD may be composed of a housing apparatus with a pair of chambers with an inlet and outlet port, at least one ventricular outflow conduit, and an actuator that contracts one of the chambers while expanding the other to provide a positive displacement pump. See, e.g., U.S. Pat. No. 6,264,601. The VAD may be composed of a pump, a chamber above the pump, and a tube that connects the pump and chamber using liquid and gas as a means for communication. See, e.g., U.S. Pat. No. 6,146,325.

In another aspect, the VAD may be a device designed specifically for the left ventricle. For example, the VAD may be a blood pump adapted to be joined in flow communication between the left ventricle and the aorta using an inlet flow pressure sensor and a controller that may adjust speed of pump based on sensor feedback. See, e.g., U.S. Pat. No. 6,623,420. The VAD may be composed of a bag adapted to expand by being filled with blood and able to contract to expel the blood, and the means for varying the resistance of the bag by using gaseous substance through a duct to a containing casing. See, e.g., U.S. Pat. No. 6,569,079. The VAD may be a pump system composed of a deformable sac with inlet and outlet means and a pair of plates on opposite sides of the sac to deform the sac. See, e.g., U.S. Pat. No. 5,599,173.

In another aspect, the VAD may be a device designed as a biventricular assist device. For example, the VAD may be a biventricular assist device composed of a self-supporting cup having an annular diaphragm that forms a fluid chamber around the heart cavity whereby it may have a pressure inlet/port that communicates with the fluid chamber to regulate positive and negative pressures. See, e.g., U.S. Pat. No. 5,908,378; 5,749,839 and 5,738,627.

In another aspect, the VAD may be an implanted system used to supplement the pumping of blood circulation from a location outside the heart. For example, the VAD may be an extracardiac pumping system composed of an inflow and outflow conduit fluidly coupled to the pump (e.g., pulsatile or rotary pump) and a control circuit to synchronously actuate the pump. See, e.g., U.S. Pat. Nos. 6,610,004; 6,428,464 and 6,200,260.

In another aspect, the VAD related devices may be a used in conjunction with VADs or as stand alone to treat congestive heart failure victims. For example, a VAD related device may be a reinforcement device composed of a jacket that is applied to the heart to constrain cardiac expansion to a predetermined limit. See, e.g., U.S. Pat. Nos. 6,582,355; 6,567,699; 6,241,654 and 6,169,922.

Representative examples of VADs, which may be combined with one or more agents according to the present invention, include commercially available products. For example, Thoratec Corporation (Pleasanton, Calif.) sells the HEARTMATE Left Ventricular Assist Systems. WorldHeart Corporation (ON, Canada) sells the WORLDHEART NOVACOR Left Ventricular Assist System. Arrow International (Reading, Pa.) sells the LIONHEART Left Ventricular Assist System.

In one aspect, the present invention provides LVAD devices that include an anti-scarring drug combination (or individual component(s) thereof) or a composition that includes an anti-scarring drug combination (or individual component(s) thereof). Numerous polymeric and non-polymeric delivery systems have been described above. These compositions can further comprise one or more fibrosis-inhibiting drug combinations (or individual components thereof) such that the overgrowth of granulation tissue is inhibited or reduced.

Methods for incorporating the fibrosis-inhibiting drug combination (or individual component(s) thereof) into or onto the device includes: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier), (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) constructing the device itself or a portion of the device with a fibrosis-inhibiting drug combination (or individual component(s) thereof) or a composition comprising a fibrosis-inhibiting drug combination (or individual component(s) thereof), or (f) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. The coatings can be applied to different portions of the device. For example, the coating can be (a) as a coating applied to the external surface of the tube that leads out of the left ventricle; (b) as a coating applied to the internal (luminal) surface of the tube that leads out of the left ventricle; (c) as a coating applied to external surface of the tube that lead to the aorta; (d) as a coating applied to internal (luminal) surface of the tube that lead to the aorta; (e) as a coating that is applied to the ends of the tube where they are in contact with the heart tissue, and/or (f) as a coating applied to all or parts of the entire device.

In addition to coating the device with the fibrosis-inhibiting drug combination (or individual component(s) thereof) or composition, the fibrosis-inhibiting drug combination (or individual component(s) thereof) can be mixed with the materials that are used to make the device such that the fibrosis-inhibiting drug combination (or individual component(s) thereof) is incorporated into the final device.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any anti-scarring drug combination (or individual component(s) thereof) described above can be utilized in the practice of this embodiment. Within one embodiment of the invention, VAD devices (e.g., LVAD's) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations (or individual components thereof) for use in left ventricular assist devices include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

As ventricular assist devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Regardless of the method of application of the drug to the device, the exemplary anti-fibrosing drug combinations (or individual components thereof)should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with ventricular assist devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm². Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

Spinal Implants

In one aspect, the present invention provides for the combination of an anti-scarring drug combination (or individual component(s) thereof) and a spinal implant (e.g., a spinal prosthesis). As used herein, the term “spinal prostheses” refers to devices that are located in, on, or near the spine and which enhance the ability of the spine to perform its function in the host. Spinal prostheses may be used to treat the vertebral column following degeneration or damage to the spine or a component or portion thereof. In healthy hosts, the vertebral column is composed of vertebral bone plates separated by intervertebral discs that form strong joints and absorb spinal compression. The intervertebral disc is comprised of an inner gel-like substance called the nucleus pulposus with surrounding tough fibrocartilagenous fibers called the annulus fibrosis. When damage occurs to the intervertebral disc, the host can develop spinal dysfunction, crippling pain, as well as long-term disability. Typically, damage to an intervertebral disc requires surgery which often results in the fusion of adjacent vertebral bone plates using various techniques and devices. Fusion of vertebral segments alleviates the pain by restricting vertebral motion at the damaged intervertebral disc. When only one vertebral segment is fused, the host will not have any noticeable motion limitations. However, when two or more segments are fused, the normal motion of the back may become limited and thus, pain relief may not resolve due to the additional stress that is induced across the remaining vertebral joints.

In one aspect, the damaged vertebral segment may be treated using a spinal prosthesis that induces fusion between the vertebral plates. This may be conducted when only one vertebral segment is damaged. In another aspect, the damaged vertebral segment may be treated using a spinal prosthesis that maintains vertebral movement within the vertebral joint. This may be conducted when damage to more than one vertebral segment occurs.

Examples of spinal prostheses include, without limitation, spinal discs and related devices including vertebral implants, vertebral disc prostheses, lumbar disc implants, cervical disc implants, intervertebral discs, implantable prostheses, spinal prostheses, artificial discs, prosthetic implants, prosthetic spinal discs, spinal disc endoprostheses, spinal implants, artificial spinal discs, intervertebral implants, implantable spinal grafts, implantable bone grafts, artificial lumbar discs, spinal nucleus implants, and intervertebral disc spacers. Also included within the term spinal prostheses are fusion cages and related devices including fusion baskets, fusion cage apparatus, interbody cages, interbody implants, fusion devices, fusion cage anchoring devices, bone fixation apparatus, bone fixation instrumentation, bone fixation devices, fusion stabilization chamber, fusion cage anchoring plates, anchoring bone plates and bone screws.

A spinal prosthesis according to the present invention may be composed of a single material or a variety of materials including, without limitation, allograft bone material (see, e.g., U.S. Pat. No. 6,143,033), metals (see, e.g., U.S. Pat. No. 4,955,908), and/or synthetic materials (see, e.g., U.S. Pat. Nos. 6,264,695, 6,419,706, 5,824,093 and 4,911,718). The prosthesis must be biocompatible. It may consist of biodegradable or non-biodegradable components depending on the intended function of the device. See, e.g., U.S. Pat. No. 4,772,287. The spinal prosthesis may be biologically inert and serve as a mechanical means of stabilizing the vertebral column (see, e.g., U.S. Pat. Nos. 4,955,908 and 5,716,415) or it may be biologically active and serve to promote fusion with the adjacent vertebral bone plates (see, e.g., U.S. Pat. Nos. 5,489,308 and 6,520,993).

In one aspect, the prosthesis may be a fusion cage designed to promote vertebral fusion in order to limit movement between adjacent vertebrae. Fusion cages may be interbody devices that fit within the intervertebral space or they may encompass both the intervertebral space and the anterior region of the vertebral column. Fusion cages may have various shapes. For example, fusion cages may be have a rectangular shape or may be cylindrical in shape and may have a plurality of openings and helical threading. Fusion cages may have an outer body and a hollow cavity that may or may not be used to insert bone growth-promoting material for stimulating bone fusion. For example, the prosthesis may be an interbody fusion cage that has an externally threaded stem projecting from a domed outer end which is fixed using an assembly of a plate, a fastener and bone screws. See, e.g., U.S. Pat. No. 6,156,037. The prosthesis may be a fusion cage with a threaded outer surface adapted for promoting fusion with bone structures when a bone-growth-inducing substance is packed into the cage body. See, e.g., U.S. Pat. Nos. 4,961,740, 5,015,247, 4,878,915 and 4,501,269. The prosthesis may be a generally tubular shell with a helical thread projecting with a plurality of pillars with holes to facilitate bone ingrowth and mechanical anchoring. See, e.g., U.S. Pat. Nos. 6,071,310 and 5,489,308. Other U.S. patents that describe the threaded spinal implant include U.S. Pat. Nos. 5,263,953, 5,458,638 and 5,026,373.

In another aspect, the prosthesis may be a bone fixation device designed to promote vertebral fusion in order to limit movement between adjacent vertebrae. For example, bone dowels, rods, hooks, wires, wedges, plates, screws and other components may be used to fix the vertebral segments into place. The fixation device may fit within the intervertebral space or it may encompass both the intervertebral space and the anterior region of the vertebral column or it may only encompass the anterior region of the vertebral column. A bone fixation device may be used with a fusion cage to assist in stabilizing the device within the intervertebral area. For example, the prosthesis may be in the form of a solid annular body having a plurality of discrete bone-engaging teeth protruding on the superior and inferior surfaces and having a central opening that may be filled with a bone growth-promoting material. See, e.g., U.S. Pat. No. 6,520,993. The prosthesis may have a disk-like body with weld-like raised parts disposed on opposite surfaces to enhance lateral stability in situ. See, e.g., U.S. Pat. No. 4,917,704. The prosthesis may be composed of opposite end pieces that maintain the height of the intervertebral space with an integral central element that is smaller in diameter wherein osteogenic material is disposed within the annular pocket between the end pieces. See, e.g., U.S. Pat. No. 6,146,420. The prosthesis may be composed of first and second side surfaces extending parallel to each other with upper and lower surfaces that engage the adjacent vertebrae. See, e.g., U.S. Pat. No. 5,716,415. The prosthesis may be a fusion stabilization chamber composed of a hollow intervertebral spacer and an end portion with at least one hole for affixing into the surrounding bone. See, e.g., U.S. Pat. No. 6,066,175. The prosthesis may be composed of a metallic body tapering conically from the ventral to the dorsal end and having a plurality of fishplates extending from opposite sides with openings for bone screws. See, e.g., U.S. Pat. No. 4,955,908. The prosthesis may be composed of a pair of plates which may have protrusions for engaging the adjacent vertebrae and an alignment device disposed between the engaging plates for separating the plates to maintain them in lordotic alignment. See, e.g., U.S. Pat. No. 6,576,016. The prosthesis may be a plurality of implants that are inserted side by side into the disc space that promote bone fusion across an intervertebral space. See, e.g., U.S. Pat. No. 5,522,899. The prosthesis may be an anchoring device composed of an anchoring plate with a central portion configured for attachment to a vertebral implant (e.g., fusion cage) and the end portions adapted to fasten in a fixed manner to a bony segment of the vertebra. See, e.g., U.S. Pat. No. 6,306,170. The prosthesis may be a bone fixation apparatus composed of a bone plate and a fastener apparatus (e.g., bone screws). See, e.g., U.S. Pat. Nos. 6,342,055, 6,454,769, 6,602,257 and 6,620,163.

In another aspect, the prosthesis may be an alternative to spinal fusion. The prosthesis may be a disc designed to provide normal movement between vertebral bone plates. The disc may be intended to mimic the natural shock absorbent function of the natural disc. The disc may be composed of a center core and end elements that support the disc against the adjacent vertebra or it may be intended to replace only a portion of the natural intervertebral disc (e.g., nucleus pulposus). For example, the disc may be in the form of an elastomeric section sandwiched between two rigid plates. See, e.g., U.S. Pat. Nos. 6,162,252; 5,534,030, 5,017,437 and 5,031,437. The disc may be an elongated prosthetic disc nucleus composed of a hydrogel core and a constraining flexible jacket that allows the core to deform and reform. See, e.g., U.S. Pat. No. 5,824,093. The disc may be composed of a rigid superior and inferior concaval-convex elements and a nuclear body which is located between the concave surfaces to permit movement. See, e.g., U.S. Pat. No. 6,156,067. The disc may be a partial spinal prosthesis composed of a core made of an elastic material such as silicone polymer or an elastomer which is covered by a casing made of a rigid material which is in contact with the adjacent vertebrae. See, e.g., U.S. Pat. No. 6,419,706. The disc may replace only the nucleus pulposus tissue by using a spinal nucleus implant comprised of a swellable, biomimetic plastic with a hydrophobic and hydrophilic phase which can be expanded in situ to conform to the natural size and shape. See, e.g., U.S. Pat. No. 6,264,695. The disc may be composed of a central core formed from a biocompatible elastomer wrapped by multi-layered laminae made from elastomer and fibers. See, e.g., U.S. Pat. No. 4,911,718. The disc may be composed of a fluid-filled inner bladder with an outer layer of strong, inert fibers intermingled with a bioresorbable material which promotes tissue ingrowth. See, e.g., U.S. Pat. No. 4,772,287.

In another aspect, the spinal implant may be a device that reduces spine compression or reduces adhesions that may form as a result to spinal surgery and/or trauma. For example, the device may be a protection device composed of a shield to fit onto at least one lamina on the posterior surface to prevent postoperative formation of adhesions to the spinal dura. See, e.g., U.S. Pat. Nos. 5,437,672 and 5,868,745 and U.S. Patent Application No. 2003/0078588. The device may be a prosthesis having a patch flange and a suture flange extending circumferentially around the patch such that the tissue underlying the patch is shielded and effectively nonadhesive to scar growth. See, e.g., U.S. Pat. No. 5,634,944. The device may be a protective intervening barrier composed of a biocompatible shield which is used following intraspinal or vertebral surgery to prevent postoperative adhesions from binding onto the spinal nerves. See, e.g., U.S. Pat. No. 4,013,078. The device may be used for neuro decompression while reducing fibroplasia proximate to the nerve tissue by having a surface topography texturized with outwardly-extending microstructures. See, e.g., U.S. Pat. No. 6,106,558 and U.S. Patent Application No. 2003/0078673.

Spinal prostheses and other spinal implants, which may be combined with one or more drugs according to the present invention, include commercially available products. Medtronic Sofamor Danek (Memphis, Tenn.) sells the fusion cage product INTERFIX Threaded Fusion Device. Centerpulse Spine-Tech (Minneapolis, Minn.) sells the BAK/C Cervical Interbody Fusion System fusion cage product and the CERVI-LOK Cervical Fixation System fixation device. Spinal Concepts (Austin, Tex.) sells the SC-ACUFIX Anterior Cervical Plate System. DePuy Spine, Inc. (Raynham, Mass.) sells the spinal discs, ACROFLEX TDR prostheses and the CHARITÉ Artificial Disc. Synthes-Stratec (Switzerland) sells the PRODISC system, including the PRODISC Cervical-C IDE disc replacement. Raymedica, Inc. (Minneapolis, Minn.) sells the PDN (PROSTHETIC DISC NUCLEUS).

Numerous polymeric and non-polymeric carrier systems that can be used in conjunction with spinal implants have been described above. Incorporation of a fibrosis-inhibiting drug combination (or individual component(s) thereof) into or onto a spinal implant can minimize fibrosis (or scarring) in the vicinity of the implant and may reduce or prevent the formation of adhesions between the implant and the surrounding tissue.

In one aspect, the present invention provides spinal implants that include an anti-scarring drug combination (or individual component(s) thereof) or a composition that includes an anti-scarring drug combination (or individual component(s) thereof) to inhibit scarring and adhesion between the device and the surrounding bone.

Methods for incorporating the anti-fibrosing drug combinations (or individual components thereof) onto or into a spinal implant include: (a) directly affixing to the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (b) directly incorporating into the device a fibrosis-inhibiting drug combination (or individual component(s) thereof) or a composition comprising a fibrosis-inhibiting drug combination (or individual component(s) thereof) (e.g., by either a spraying process or dipping process as described above, with or without a carrier, (c) by coating the device with a substance such as a hydrogel which will in turn absorb a fibrosis-inhibiting drug combination (or individual component(s) thereof) or a composition comprising a fibrosis-inhibiting drug combination (or individual component(s) thereof), (d) by interweaving a fibrosis-inhibiting drug combination (or individual component(s) thereof) or a composition comprising a fibrosis-inhibiting drug combination (or individual component(s) thereof) coated thread (or the polymer itself formed into a thread) into the device structure, (e) by binding film or mesh which is comprised of or coated with a fibrosis-inhibiting drug combination (or individual component(s) thereof) or a composition comprising a fibrosis-inhibiting drug combination (or individual component(s) thereof) to the spinal prosthesis, (f) constructing the device itself or a portion of the device with a fibrosis-inhibiting drug combination (or individual component(s) thereof) or a composition comprising a fibrosis-inhibiting drug combination (or individual component(s) thereof), or (g) by covalently binding a fibrosis-inhibiting drug combination (or individual component(s) thereof) or a composition comprising a fibrosis-inhibiting drug combination (or individual component(s) thereof) directly to the device surface or to a linker (small molecule or polymer) that is coated or attached to the device surface. For these devices, the coating process can be performed in such a manner as to a) coat the exterior surfaces of the device, b) coat the interior surfaces of the device or c) coat all or parts of both external and internal surface of the device.

In one aspect, a spinal implant (e.g., an implantable cages or disc) is coated with an anti-scarring drug combination (or individual component(s) thereof) or a composition that includes the anti-scarring drug combination (or individual component(s) thereof). In certain aspects, the spinal implant may be coated with (or adapted to contain) an anti-scarring drug combination (or individual component(s) thereof) on one part of the device and a fibrosis-inducing agent (e.g., silk or talc) on another part of the device. For example, the outer surface of the implant (e.g., a vertebral implant) may be coated with a fibrosis-inducing agent to improve adhesion between the device and the surrounding tissue, while the interior of the device may be coated with an anti-scarring drug combination (or individual component(s) thereof) to minimize adhesion of tissue to the interior of the implant. Examples of fibrosis-inducing agents and methods of using fibrosis-inducing agents in combination with spinal implants are described in co-pending application entitled, “Medical Implants and Fibrosis-Inducing Agents,” filed November 20, 2003 (U.S. Ser. No. 60/524,023) and Jun. 9, 2004 (U.S. Ser. No. 60/578,471).

In addition to coating the device with the anti-fibrosing drug combination (or individual component(s) thereof) or composition comprising the anti-fibrosis drug combination (or individual component(s) thereof), the anti-fibrosing drug combination (or individual component(s) thereof) can be mixed with the materials that are used to make the device such that the anti-fibrosing drug combination (or individual component(s) thereof) is incorporated into the final device.

In addition to applying the fibrosis agent to the spinal implant, an in situ forming composition, gel or thermogel composition that further comprises a fibrosis-inhibiting agent can be applied to the placement site of the spinal prosthesis, (a) prior to placement of the prosthesis, (b) after placement of the prosthesis and/or (c) both prior and post placement on the prosthesis.

For the in situ forming, thermogel and gel compositions, the fibrosis-inhibiting agents can be incorporated directly into the formulation to produced a suspension or a solution or it can be incorporated into a secondary carrier (e.g., micelles, liposomes, microspheres, microparticles, nanospheres, microparticulates, emulsions and/or microemulations) that is then incorporated into the in situ forming compositions. In another embodiment, the fibrosis-inhibiting drug combination (or individual component(s) thereof) can be electrostatically or covalently bound to one or more of the polymeric components of the in situ forming composition.

In another embodiment, the fibrosis-inhibiting drug combination (or individual component(s) thereof) can be incorporated into a biodegradable or dissolvable film or mesh that is then applied to the treatment site prior or post implantation of the prosthesis/implant. Preferred materials for the manufacture of these films or meshes are hyaluronic acid (crosslinked or non-crosslinked), cellulose derivatives (e.g., hydroxypropyl cellulose), PLGA, POLYACTIVE, collagen and crosslinked poly(ethylene glycol).

In another embodiment, a solution or suspension that further comprises a fibrosis-inhibiting drug combination (or individual component(s) thereof) can be applied to the placement site of the spinal prosthesis, (a) prior to placement of the prosthesis, (b) after placement of the prosthesis and/or (c) both prior and post placement on the prosthesis. The fibrosis-inhibiting drug combinations (or individual components thereof) can be incorporated directly into the formulation to produced a suspension or a solution or it can be incorporated into a secondary carrier (e.g., micelles, liposomes, microspheres, microparticles, nanospheres, microparticulates, emulsions and/or microemulations) that is then incorporated into the in situ forming compositions. This solution or suspension can be applied (sprayed, rubbed, dripped etc) onto the treatment are prior to or post prosthesis placement.

In addition to incorporation of a fibrosis-inhibiting drug combination (or individual component(s) thereof) into or onto the device, another biologically active agent can be incorporated into or onto the device, for example an anti-inflammatory (e.g., dexamethazone or aspirin), antithrombotic agent (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any adhesion or fibrosis-inducing drug combinations (or individual components thereof) described above can be utilized in the practice of this embodiment. Within one embodiment of the invention, spinal implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations (or individual components thereof) for use in spinal implants include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

As spinal implants are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days. It should be understood in certain embodiments that within the drug combination, one drug may be released at a different rate and/or for a different amount of time than the other drug(s).

Regardless of the method of application of the drug to the device, the exemplary anti-fibrosing drug combinations (or individual components thereof), used alone or in combination, should be administered under the following dosing guidelines. The total amount (dose) of anti-scarring agent(s) in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of device surface to which the agent(s) are applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring drug combinations (or individual components thereof) that can be used in conjunction with spinal implants and devices in accordance with the invention.

Exemplary anti-fibrotic drug combinations for dose explanation purposes include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof. Total dose of each drug within the combinations generally do not exceed 500 mg (range of 0.1 ug to 500 mg; preferred 1 ug to 200 mg). Concentration of each drug within the combinations generally does not exceed 500 mg/ml (range of 0.01 ug/ml to 500 mg/ml; preferred 1 ug/ml to 200 mg/ml). Volume administered of formulation is generally between 0.05 ml and 10 ml, preferred 0.1 ml to 5 ml. Dose per unit area is generally between 0.01 ug-200 ug per mm², preferably from 0.1 ug/mm² to 100 ug/mm₂. Minimum concentration of 10⁻⁸ to 10⁻⁴ M of each drug is to be maintained on the implant or barrier surface. Ratio of each drug in the combination generally is within the range of 1:1 to 1:1000. Molar ratios within this range may include, but are not limited to, 1:5, 1:10, 1:15, 1:20, 1:30, 1:50, 1:75, 1:100, 1:200, 1:500, and 1:1000.

(g) Infiltration of Anti-fibrosis Drug Combinations Around Medical Devices or Implants

Another use of the drug combinations (or individual components thereof) and compositions drug combinations (or individual components thereof) described herein may be to infiltrate the drug combinations or compositions into tissue adjacent to a medical device.

The drug combination (or individual component(s) thereof) or the composition comprising the drug combination (or individual component(s) thereof) of the present invention may be infiltrated around implanted medical devices by applying the drug combination (or individual component(s) thereof) or composition directly and/or indirectly into and/or onto (a) tissue adjacent to the medical device; (b) the vicinity of the medical device-tissue interface; (c) the region around the medical device; and (d) tissue surrounding the medical device. Methods for infiltrating the subject drug combinations (or individual components thereof) or compositions into tissue adjacent to a medical device include delivering the drug combinations or compositions: (a) to the medical device surface (e.g., as an injectable, paste, gel or mesh) during the implantation procedure; (b) to the surface of the tissue (e.g., as an injectable, paste, gel, in situ forming gel or mesh) immediately prior to, or during, implantation of the medical device; (c) to the surface of the medical device and/or the tissue surrounding the implanted medical device (e.g., as an injectable, paste, gel, in situ forming gel or mesh) immediately after the implantation of the medical device; (d) by topical application of the drug combination (or individual component(s) thereof) or the composition comprising the drug combination (or individual component(s) thereof) into the anatomical space where the medical device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the medical device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

Representative examples of compositions that may be loaded anti-fibrosis drug combinations (or individual components thereof) and infiltrated into tissue adjacent to a medical device include: (a) sprayable collagen-containing formulations such as COSTASIS (Angiotech Pharmaceuticals, Inc., Canada) and crosslinked poly(ethylene glycol)—methylated collagen compositions (described, e.g., in U.S. Pat. Nos. 5,874,500 and 5,565,519) infiltrated into tissue adjacent to the medical device; (b) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.) infiltrated into tissue adjacent to the medical device; (c) fibrinogen-containing formulations such as FLOSEAL or TISSEAL (both from Baxter Healthcare Corporation, Fremont, Calif.) infiltrated into tissue adjacent to the medical device; (d) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation) infiltrated into tissue adjacent to the medical device; (e) polymeric gels for surgical implantation such as REPEL (Life Medical Sciences, Inc., Princeton, N.J.) or FLOWGEL (Baxter Healthcare Corporation) infiltrated into tissue adjacent to the medical device; (f) orthopedic “cements” used to hold prostheses and tissues in place infiltrated into tissue adjacent to the medical device, such as OSTEOBOND (Zimmer, Inc., Warsaw, Ind.), low viscosity cement (LVC); Wright Medical Technology, Inc., Arlington, Tenn.), SIMPLEX P (Stryker Corporation, Kalamazoo, Mich.), PALACOS (Smith & Nephew Corporation, United Kingdom), and ENDURANCE (Johnson & Johnson, Inc., New Brunswick, N.J.); (g) surgical adhesives containing cyanoacrylates such as DERMABOND (Johnson & Johnson, Inc.), INDERMIL (U.S. Surgical Company, Norwalk, Conn.), GLUSTITCH (Blacklock Medical Products Inc., Canada), TISSUEMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.) infiltrated into tissue adjacent to the medical device; (h) implants containing hydroxyapatite (or synthetic bone material such as calcium sulfate, VITOSS and CORTOSS (both from Orthovita, Inc., Malvern, Pa.) infiltrated into tissue adjacent to the medical device; (i) other biocompatible tissue fillers, such as those made by BioCure, Inc. (Norcross, Ga.), 3M Company (St. Paul, Minn.) and Neomend, Inc. (Sunnyvale, Calif.) infiltrated into tissue adjacent to the medical device; (j) polysacharride gels such as the ADCON series of gels (available from Gliatech, Inc., Cleveland, Ohio.) infiltrated into tissue adjacent to the medical device; and/or (k) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.) infiltrated into tissue adjacent to the medical device.

Other examples of compositions that may be infiltrated into tissue adjacent to a medical device include compositions formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix.

Representative examples of medical devices for use with the subject compositions are described below.

Intravascular Devices

In one aspect, the present invention provides therapeutic agents or pharmaceutical compositions that may be infiltrated into the tissue adjacent to the intravascular devices (e.g., anastomotic connectors, stents, drug-delivery balloons, intravascular catheters), where the polymeric composition may include an anti-fibrosis drug combination (or individual component(s) thereof). Examples of intravascular devices are provided above in conjunction with the coating of medical devices. Numerous agents or compositions for use with intravascular devices have been described above which may be infiltrated into the tissue adjacent to the device (preferably near the device-tissue interface).

Anti-fibrosis drug combinations (or individual components thereof) or compositions that comprise the drug combinations (or individual components thereof) may be infiltrated around implanted intravascular devices by applying the composition directly and/or indirectly into and/or onto (a) tissue adjacent to the intravascular device; (b) the vicinity of the intravascular device-tissue interface; (c) the region around the intravascular device; and (d) tissue surrounding the intravascular device.

Methods for infiltrating a drug combination (or individual component(s) thereof) or a composition comprising a drug combination (or individual component(s) thereof) into tissue adjacent to an intravascular device include delivering the agent or composition: (a) to the intravascular device surface (e.g., as an injectable, paste, gel or mesh) during the implantation procedure; (b) to the surface of the tissue (e.g., as an injectable, paste, gel, in situ forming gel or mesh) immediately prior to, or during, implantation of the intravascular device; (c) to the surface of the intravascular device and/or the tissue surrounding the implanted intravascular device (e.g., as an injectable, paste, gel, in situ forming gel or mesh) immediately after the implantation of the intravascular device; (d) by topical application of the drug combination (or individual component(s) thereof) or the composition comprising the drug combination (or individual component(s) thereof) into the anatomical space where the intravascular device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the intravascular device may be inserted); (e) via percutaneous injection into the tissue surrounding the intravascular device as a solution as an infusate or as a sustained release preparation; (f) by any combination of the aforementioned methods. Combination therapies (e.g., combinations with antithrombotic and/or antiplatelet agents) may also be used. In all cases it is understood that the subject agents or compositions may be infiltrated into tissue adjacent to all or a portion of the device.

According to one aspect, any fibrosis-inhibiting drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to intravascular devices inhibit one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the drug combination (or individual component(s) thereof) or the composition comprising the drug combination (or individual component(s) thereof) for prevention or inhibition of fibrosis in accordance with the present invention will depend on a variety of factors, including the type of formulation, the location of the treatment site, and the type of condition being treated. As intravascular devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing drug combinations (or individual components thereof) or compositions comprising drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

In certain embodiments, any anti-infective agent described above may be used in combination with the anti-fibrosis drug combination (or individual component(s) thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present agents or compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Gastrointestinal Stents

In one aspect, the anti-fibrosis drug combinations (or individual components thereof) or pharmaceutical compositions that comprise the anti-fibrosis drug combinations (or individual components thereof) of the present invention may be infiltrated into tissue adjacent to a gastrointestinal (GI) stent. Examples of gastrointestinal stents are provided above in conjunction with the coating of medical devices. Numerous agents or compositions for use with intravascular devices have been described above which may be infiltrated into the tissue adjacent to the device (preferably near the device-tissue interface).

Anti-fibrosis drug combinations (or individual components thereof) or pharmaceutical compositions that comprise the anti-fibrosis drug combinations (or individual components thereof) may be infiltrated around implanted GI stents by applying the composition directly and/or indirectly into and/or onto (a) tissue adjacent to the GI stent; (b) the vicinity of the GI stent-tissue interface; (c) the region around the GI stent; and (d) tissue surrounding the GI stent.

Methods for infiltrating the anti-fibrosis drug combinations (or individual components thereof) or pharmaceutical compositions that comprise the anti-fibrosis drug combinations (or individual components thereof) into tissue adjacent to a GI stent include delivering the drug combination (or individual component(s) thereof) or the composition comprising the drug combination (or individual component(s) thereof): (a) to the GI stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the GI stent; (c) to the surface of the GI stent and/or the tissue surrounding the implanted GI stent (e.g., as an injectable, paste, gel, in situ forming gel or mesh) immediately after the implantation of the GI stent; (d) by topical application of the drug combination (or individual component(s) thereof) or the composition comprising the drug combination (or individual component(s) thereof) into the anatomical space where the GI stent may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the GI stent as a solution as an infusate or as a sustained release preparation; (f) by any combination of the aforementioned methods. Combination therapies (e.g., combinations with antithrombotic and/or antiplatelet agents) may also be used. In all cases it is understood that the subject agents or compositions may be infiltrated into tissue adjacent to all or a portion of the device.

According to one aspect, any anti-fibrosis drug combinations (or individual components thereof) or combinations comprising anti-fibrosis drug combinations (or individual components thereof) described above may be utilized in the practice of the present invention. In one aspect of the invention, the drug combinations (or individual components thereof) or the compositions comprising the drug combinations (or individual components thereof) infiltrated into tissue adjacent to GI stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present anti-fibrosis drug combinations (or individual components thereof) or pharmaceutical compositions that comprise the anti-fibrosis drug combinations (or individual components thereof) for prevention or inhibition of fibrosis in accordance with the present invention will depend on a variety of factors, including the type of formulation, the location of the treatment site, and the type of condition being treated. As GI stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination of an anti-fibrosis drug combination (or individual component(s) thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present agents or compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Tracheal and Bronchial Stents

The present invention provides for infiltration of an anti-fibrosis drug combinations (or individual components thereof) or pharmaceutical compositions that comprise the anti-fibrosis drug combinations (or individual components thereof) into tissue adjacent to a tracheal or bronchial stent device. Representative examples of tracheal or bronchial stents that may benefit from having the subject compositions infiltrated into adjacent tissue tracheal and bronchial stents are provided above in conjunction with the coating of medical devices. Numerous agents or compositions for use with tracheal and bronchial have been described above which may be infiltrated into the tissue adjacent to the device (preferably near the device-tissue interface).

Anti-fibrosis drug combinations (or individual components thereof) or pharmaceutical compositions that comprise the anti-fibrosis drug combinations (or individual components thereof) may be infiltrated around implanted tracheal and bronchial stents by applying the composition directly and/or indirectly into and/or onto (a) tissue adjacent to the tracheal/bronchial stent; (b) the vicinity of the tracheal/bronchial stent-tissue interface; (c) the region around the tracheal/bronchial stent; and (d) tissue surrounding the tracheal/bronchial stent.

Methods for infiltrating the anti-fibrosis drug combinations (or individual components thereof) or pharmaceutical compositions that comprise the anti-fibrosis drug combinations (or individual components thereof) into tissue adjacent to a tracheal/bronchial stent include delivering the drug combination (or individual component(s) thereof) or the composition comprising the drug combination (or individual component(s) thereof): (a) to the tracheal/bronchial stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the tracheal/bronchial stent; (c) to the surface of the tracheal/bronchial stent and/or the tissue surrounding the implanted tracheal/bronchial stent (e.g., as an injectable, paste, gel, in situ forming gel or mesh) immediately after the implantation of the tracheal/bronchial stent; (d) by topical application of the drug combination (or individual component(s) thereof) or the composition comprising the drug combination (or individual component(s) thereof) into the anatomical space where the tracheal/bronchial stent may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the tracheal/bronchial stent as a solution as an infusate or as a sustained release preparation; (f) by any combination of the aforementioned methods. Combination therapies (e.g., combinations with antithrombotic and/or antiplatelet agents) may also be used. In all cases it is understood that the anti-fibrosis drug combinations (or individual components thereof) or pharmaceutical compositions that comprise the anti-fibrosis drug combinations (or individual components thereof) may be infiltrated into tissue adjacent to all or a portion of the device.

According to one aspect, any anti-fibrosis drug combinations (or individual components thereof)or pharmaceutical compositions that comprise the anti-fibrosis drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to tracheal and bronchial stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations (or individual components thereof) or pharmaceutical compositions that comprise the anti-fibrosis drug combinations (or individual components thereof) for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from anti-fibrosis drug combinations (or individual components thereof) or pharmaceutical compositions that comprise the anti-fibrosis drug combinations (or individual components thereof) for prevention or inhibition of fibrosis in accordance with the present invention will depend on a variety of factors, including the type of formulation, the location of the treatment site, and the type of condition being treated. As tracheal and bronchial stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or pharmaceutical compositions that comprise the anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 1 μg/mm², or about 250 μg/mm²-1000 μg/mm², or about 1000 μg/mm²-2500 μg/mm².

According to another aspect, any anti-infective agent described above may be used in combination with an anti-fibrosis drug combination (or individual component(s) thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm-1000 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M about 10⁻⁶ M to 10⁻⁵ M or about 10⁻⁵ M to 10⁻⁴ M of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Genital-Urinary Stents

In one aspect, the drug combination (or individual component(s) thereof) or the composition comprising the drug combination (or individual component(s) thereof) may be infiltrated into tissue adjacent to a genital-urinary (GU) stent device.

Representative examples genital-urinary (GU) stents that may benefit from having the subject compositions infiltrated into adjacent tissue are provided above in conjunction with the coating of medical devices. Numerous polymeric compositions for use with tracheal and bronchial have been described above which may be infiltrated into the tissue adjacent to the device (preferably near the device-tissue interface).

The drug combination (or individual component(s) thereof) or the composition comprising the drug combination (or individual component(s) thereof) may be infiltrated around implanted GU stents by applying the composition directly and/or indirectly into and/or onto (a) tissue adjacent to the GU stent; (b) the vicinity of the GU stent-tissue interface; (c) the region around the GU stent; and (d) tissue surrounding the GU stent.

Methods for infiltrating the drug combination (or individual component(s) thereof) or the composition comprising the drug combination (or individual component(s) thereof) into tissue adjacent to a GU stent include delivering the drug combination (or individual component(s) thereof) or the composition comprising the drug combination (or individual component(s) thereof): (a) to the GU stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the GU stent; (c) to the surface of the GU stent and/or the tissue surrounding the implanted GU stent (e.g., as an injectable, paste, gel, in situ forming gel or mesh) immediately after the implantation of the GU stent; (d) by topical application of the drug combination (or individual component(s) thereof) or the composition comprising the drug combination (or individual component(s) thereof) into the anatomical space where the GU stent may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the GU stent as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the subject compositions may be infiltrated into tissue adjacent to all or a portion of the device.

According to one aspect, any drug combination (or individual component(s) thereof) or any composition comprising the drug combination (or individual component(s) thereof) described above may be utilized in the practice of the present invention. In one aspect of the invention, the drug combination (or individual component(s) thereof) or the composition comprising the drug combination (or individual component(s) thereof) infiltrated into tissue adjacent to GU stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for prevention or inhibition of fibrosis in accordance with the present invention will depend on a variety of factors, including the type of formulation, the location of the treatment site, and the type of condition being treated. As GU stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary drug combinations (or individual components thereof) or the compositions comprising the drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-10 μg/mm², or about 10 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Ear and Nose and Throat Stents

In one aspect, the present anti-fibrosis drug combinations (or individual components thereof) or pharmaceutical compositions that comprise the anti-fibrosis drug combinations (or individual components thereof) may be infiltrated into tissue adjacent to an ear-nose-throat (ENT) stent device (e.g., a lacrimal duct stent, Eustachian tube stent, nasal stent, or sinus stent).

Representative examples ear and nose stents that may benefit from having the subject compositions infiltrated into adjacent tissue are provided above in conjunction with the coating of medical devices. Numerous polymeric compositions for use with tracheal and bronchial have been described above which may be infiltrated into the tissue adjacent to the device (preferably near the device-tissue interface).

Anti-fibrosis drug combinations (or individual components thereof) or pharmaceutical compositions that comprise the anti-fibrosis drug combinations (or individual components thereof) may be infiltrated around implanted ENT stents by applying the composition directly and/or indirectly into and/or onto (a) tissue adjacent to the ENT stent; (b) the vicinity of the ENT stent-tissue interface; (c) the region around the ENT stent; and (d) tissue surrounding the ENT stent.

Methods for infiltrating the anti-fibrosis drug combinations (or individual components thereof) or pharmaceutical compositions that comprise the anti-fibrosis drug combinations (or individual components thereof) into tissue adjacent to a ENT stent include delivering the drug combination (or individual component(s) thereof) or the composition comprising the drug combination (or individual component(s) thereof): (a) to the ENT stent surface (e.g., as an injectable, paste, gel or mesh) during the implantation procedure; (b) to the surface of the tissue (e.g., as an injectable, paste, gel, in situ forming gel or mesh) immediately prior to, or during, implantation of the ENT stent; (c) to the surface of the ENT stent and/or the tissue surrounding the implanted ENT stent (e.g., as an injectable, paste, gel, in situ forming gel or mesh) immediately after the implantation of the ENT stent; (d) by topical application of the drug combination (or individual component(s) thereof) or the composition comprising the drug combination (or individual component(s) thereof) into the anatomical space where the ENT stent may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the ENT stent as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or pharmaceutical compositions that comprise the anti-fibrosis drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to ENT stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations (or individual components thereof) for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or pharmaceutical compositions that comprise the anti-fibrosis drug combinations (or individual components thereof) for prevention or inhibition of fibrosis in accordance with the present invention will depend on a variety of factors, including the type of formulation, the location of the treatment site, and the type of condition being treated. As ENT stents are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing drug combinations (or individual components thereof) or pharmaceutical compositions that comprise the anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with anti-fibrosis drug combinations (or individual components thereof) or pharmaceutical compositions that comprise the anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Ear Ventilation Tubes

In another aspect, anti-fibrosis drug combinations (or individual components thereof) or pharmaceutical compositions that comprise the anti-fibrosis drug combinations (or individual components thereof) may be infiltrated into tissue adjacent to an ear ventilation tube (also referred to as a tympanostomy tube).

Representative examples ear ventilation tubes that may benefit from having the subject compositions infiltrated into adjacent tissue are provided above in conjunction with the coating of medical devices. Numerous agents or compositions for use with ear ventilation tubes have been described above which may be infiltrated into the tissue adjacent to the device (preferably near the device-tissue interface).

Anti-fibrosis drug combinations (or individual components thereof) or pharmaceutical compositions that comprise the anti-fibrosis drug combinations (or individual components thereof) may be infiltrated around implanted ear ventilation tube devices by applying the composition directly and/or indirectly into and/or onto (a) tissue adjacent to the ear ventilation tube devices; (b) the vicinity of the ear ventilation tube device-tissue interface; (c) the region around the ear ventilation tube device; and (d) tissue surrounding the ear ventilation tube device.

Methods for infiltrating the subject compositions into tissue adjacent to an ear ventilation tube device include delivering the drug combination (or individual component(s) thereof) or the composition comprising the drug combination (or individual component(s) thereof): (a) to the ear ventilation tube device surface (e.g., as an injectable, paste, gel or mesh) during the implantation procedure; (b) to the surface of the tissue (e.g., as an injectable, paste, gel, in situ forming gel or mesh) immediately prior to, or during, implantation of the ear ventilation tube device; (c) to the surface of the ear ventilation tube device and/or the tissue surrounding the implanted ear ventilation tube device (e.g., as an injectable, paste, gel, in situ forming gel or mesh) immediately after the implantation of the ear ventilation tube device; (d) by topical application of the drug combination (or individual component(s) thereof) or the composition comprising the drug combination (or individual component(s) thereof) into the anatomical space where the ear ventilation tube device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the ear ventilation tube device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or pharmaceutical compositions that comprise the anti-fibrosis drug combinations (or individual components thereof) described above can be utilized in the practice of the present invention. In one aspect of the invention, the anti-fibrosis drug combinations (or individual components thereof) or pharmaceutical compositions that comprise the anti-fibrosis drug combinations (or individual components thereof) infiltrated into tissue adjacent to ear ventilation tubes may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting anti-fibrosis drug combinations (or individual components thereof) or pharmaceutical compositions that comprise the anti-fibrosis drug combinations (or individual components thereof) for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or pharmaceutical compositions that comprise the anti-fibrosis drug combinations (or individual components thereof) in accordance with the present invention will depend on a variety of factors, including the type of formulation, the location of the treatment site, and the type of condition being treated. As ear ventilation tubes are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing agents, used alone or in combination, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Intraocular Implants

In another aspect, the anti-fibrosis drug combinations (or individual components thereof) or pharmaceutical compositions that comprise the anti-fibrosis drug combinations (or individual components thereof) may be infiltrated into tissue adjacent to an intraocular implant.

Representative examples of intraocular implants that may benefit from having the subject compositions infiltrated into adjacent tissue are provided above in conjunction with the coating of medical devices. Numerous agents or compositions for use with intraocular implants have been described above which may be infiltrated into the tissue adjacent to the device (preferably near the device-tissue interface).

Anti-fibrosis drug combinations (or individual components thereof) or pharmaceutical compositions that comprise the anti-fibrosis drug combinations (or individual components thereof) may be infiltrated around implanted intraocular implants by applying the composition directly and/or indirectly into and/or onto (a) tissue adjacent to the intraocular implant; (b) the vicinity of the intraocular implant-tissue interface; (c) the region around the intraocular implant; and (d) tissue surrounding the intraocular implant.

Methods for infiltrating the drug combination (or individual component(s) thereof) or the composition comprising the drug combination (or individual component(s) thereof) into tissue adjacent to an intraocular implant include delivering the drug combination (or individual component(s) thereof) or the composition comprising the drug combination (or individual component(s) thereof): (a) to the intraocular implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the intraocular implant; (c) to the surface of the intraocular implant and/or the tissue surrounding the implanted intraocular implant (e.g., as an injectable, paste, gel, in situ forming gel or mesh) immediately after the implantation of the intraocular implant; (d) by topical application of the drug combination (or individual component(s) thereof) or the composition comprising the drug combination (or individual component(s) thereof) into the anatomical space where the intraocular implant may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the intraocular implant as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

The process of infiltrating the subject compositions into tissue adjacent to these implants and the materials selected for these processes are such that they do not significantly alter the refractive index of the intraocular implant or the visible light transmission of the implant or lens.

According to one aspect, any anti-fibrosis drug combinations (or individual components thereof) or pharmaceutical compositions that comprise the anti-fibrosis drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to intraocular implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for prevention or inhibition of fibrosis in accordance with the present invention will depend on a variety of factors, including the type of formulation, the location of the treatment site, and the type of condition being treated. As intraocular implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or pharmaceutical compositions that comprise the anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with anti-fibrosis drug combinations (or individual components thereof) or pharmaceutical compositions that comprise the anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Hypertrophic Scars and Keloids

In another aspect, the anti-fibrosis drug combinations (or individual components thereof) or pharmaceutical compositions that comprise the anti-fibrosis drug combinations (or individual components thereof) may be infiltrated into tissue adjacent to a device for use in treating hypertrophic scars and keloids.

Representative examples of implants for use in treating hypertrophic scars and keloids that may benefit from having the subject compositions infiltrated into adjacent tissue are provided above in conjunction with the coating of medical devices. Numerous agents or compositions for use with hypertrophic scar and keloid implants have been described above which may be infiltrated into the tissue adjacent to the device (preferably near the device-tissue interface).

The compositions may be a topical or injectable composition that includes an anti-scarring drug combinations (or individual components thereof) and a polymeric carrier suitable for application on or into hypertrophic scars or keloids. Incorporation of a fibrosis-inhibiting drug combinations (or individual components thereof) into a topical formulation or an injectable formulation is one approach to treat this condition. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are described above.

Anti-fibrosis drug combinations (or individual components thereof) or compositions that comprise drug combinations (or individual components thereof) may be infiltrated around devices used for hypertrophic scars and keloids by applying the composition directly and/or indirectly into and/or onto (a) tissue adjacent to the device used for hypertrophic scars and keloids; (b) the vicinity of the tissue interface with the device used for hypertrophic scars and keloids; (c) the region around the device used for hypertrophic scars and keloids; and (d) tissue surrounding the device used for hypertrophic scars and keloids.

Methods for infiltrating the subject anti-fibrosis drug combinations (or individual components thereof) or compositions that comprise drug combinations (or individual components thereof) into tissue adjacent to a device used for hypertrophic scars and keloids include delivering the drug combination (or individual component(s) thereof) or the composition comprising the drug combination (or individual component(s) thereof): (a) to the surface of the device used for hypertrophic scars and keloids (e.g., as an injectable, paste, gel or mesh) during the implantation procedure; (b) to the surface of the tissue (e.g., as an injectable, paste, gel, in situ forming gel or mesh) immediately prior to, or during, implantation of the device used for hypertrophic scars and keloids; (c) to the surface of the device used for hypertrophic scars and keloids and/or the tissue surrounding the implanted device used for hypertrophic scars and keloids (e.g., as an injectable, paste, gel, in situ forming gel or mesh) immediately after the implantation of the device used for hypertrophic scars and keloids; (d) by topical application of the drug combination (or individual component(s) thereof) or the composition comprising the drug combination (or individual component(s) thereof) into the anatomical space where the device used for hypertrophic scars and keloids may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the device used for hypertrophic scars and keloids as a solution as an infusate or as a sustained release preparation; (f) by any combination of the aforementioned methods. Combination therapies (e.g., combinations with antithrombotic and/or antiplatelet agents) may also be used. In all cases it is understood that the Anti-fibrosis drug combinations (or individual components thereof) or compositions that comprise drug combinations (or individual components thereof) may be infiltrated into tissue adjacent to all or a portion of the device.

According to one aspect, any anti-fibrosis drug combinations (or individual components thereof) or compositions that comprise drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to devices for the treatment of hypertrophic scars and keloids may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present anti-fibrosis drug combinations (or individual components thereof) or compositions that comprise drug combinations (or individual components thereof) for prevention or inhibition of fibrosis in accordance with the present invention will depend on a variety of factors, including the type of formulation, the location of the treatment site, and the type of condition being treated. As devices for the treatment of hypertrophic scars and keloids are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing drug combinations (or individual components thereof)should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 100 μg/mm²-2500 μg/mm².

According to another aspect, any anti-infective agent described above may be used in combination with anti-fibrosis drug combinations (or individual components thereof) or compositions that comprise drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Vascular Grafts

In one aspect, The present invention provides for infiltration of the anti-fibrosis drug combinations (or individual components thereof) or compositions that comprise drug combinations (or individual components thereof) into tissue adjacent to a vascular graft. Vascular graft devices having anti-fibrosis drug combinations (or individual components thereof) or compositions that comprise drug combinations (or individual components thereof) infiltrated into adjacent tissue are capable of inhibiting or reducing the overgrowth of granulation tissue and/or inhibiting or preventing infection, which can improve the clinical efficacy of these devices.

Representative examples of vascular grafts that may benefit from having the subject compositions infiltrated into adjacent tissue are provided above in conjunction with the coating of medical devices. Numerous agents or compositions for use with vascular grafts have been described above which may be infiltrated into the tissue adjacent to the device (preferably near the device-tissue interface).

Anti-fibrosis drug combinations (or individual components thereof) or compositions that comprise drug combinations (or individual components thereof) maybe infiltrated around implanted vascular grafts by applying the composition directly and/or indirectly into and/or onto (a) tissue adjacent to the vascular graft; (b) the vicinity of the vascular graft-tissue interface; (c) the region around the vascular graft; and (d) tissue surrounding the vascular graft.

Methods for infiltrating the drug combination (or individual component(s) thereof) or the composition comprising the drug combination (or individual component(s) thereof) into tissue adjacent to a vascular graft include delivering the drug combination (or individual component(s) thereof) or the composition comprising the drug combination (or individual component(s) thereof): (a) to the vascular graft surface (e.g., as an injectable, paste, gel or mesh) during the implantation procedure; (b) to the surface of the tissue (e.g., as an injectable, paste, gel, in situ forming gel or mesh) immediately prior to, or during, implantation of the vascular graft; (c) to the surface of the vascular graft and/or the tissue surrounding the implanted vascular graft (e.g., as an injectable, paste, gel, in situ forming gel or mesh) immediately after the implantation of the vascular graft; (d) by topical application of the drug combination (or individual component(s) thereof) or the composition comprising the drug combination (or individual component(s) thereof) into the anatomical space where the vascular graft may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the vascular graft as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

In addition to the fibrosis-inhibiting drug combinations (or individual components thereof), the compositions infiltrated into tissue adjacent to vascular graft devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin). The combination of agents may be contained in the composition infiltrated into tissue adjacent to the vascular graft such that the thrombogenicity and/or fibrosis is reduced or inhibited. In certain embodiments, these agents may be contained in biodegradable polymers. For example, polymeric material that forms a gel in the pores and/or on the surface of the graft may be used, such as alginates, chitosan and chitosan sulfate, hyaluronic acid, dextran sulfate, PLURONIC polymers, chain extended PLURONIC polymers, polyester-polyether block copolymers of the various configurations (e.g., MePEG-PLA, PLA-PEG-PLA, and the like).

According to one aspect, any anti-fibrosis drug combinations (or individual components thereof) or compositions that comprise drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to vascular grafts may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions that comprise drug combinations (or individual components thereof) for prevention or inhibition of fibrosis in accordance with the present invention will depend on a variety of factors, including the type of formulation, the location of the treatment site, and the type of condition being treated. As vascular grafts are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination of the anti-fibrosis drug combinations (or individual components thereof) or compositions that comprise drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01μg/mm²-1 μg/mm², or about 1 μg/mm²-10 μg/mm², or about 10 μg/mm²-100 μg/mm², or about 100 μg/mm 2to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Hemodialysis Access Devices

In one aspect, the anti-fibrosis drug combinations (or individual components thereof) or compositions that comprise drug combinations (or individual components thereof) may be infiltrated into tissue adjacent to a hemodialysis access device. Hemodialysis dialysis access devices that include anti-fibrosis drug combinations (or individual components thereof) or compositions that comprise drug combinations (or individual components thereof) are capable of inhibiting or reducing the overgrowth of granulation tissue and/or inhibiting or preventing infection, which can improve the clinical efficacy of these devices.

Representative examples of hemodialysis access devices that may benefit from having the anti-fibrosis drug combinations (or individual components thereof) or compositions that comprise drug combinations (or individual components thereof) infiltrated into adjacent tissue are provided above in conjunction with the coating of medical devices. Numerous agents or compositions for use with hemodialysis access devices have been described above which may be infiltrated into the tissue adjacent to the device (preferably near the device-tissue interface).

Anti-fibrosis drug combinations (or individual components thereof) or compositions that comprise drug combinations (or individual components thereof) may be infiltrated around implanted hemodialysis access devices by applying the composition directly and/or indirectly into and/or onto (a) tissue adjacent to the hemodialysis access device; (b) the vicinity of the hemodialysis access device -tissue interface; (c) the region around the hemodialysis access device; and (d) tissue surrounding the hemodialysis access device.

Methods for infiltrating the anti-fibrosis drug combinations (or individual components thereof) or compositions that comprise drug combinations (or individual components thereof) into tissue adjacent to a hemodialysis access device include delivering the drug combination (or individual component(s) thereof) or the composition comprising the drug combination (or individual component(s) thereof): (a) to the hemodialysis access device surface (e.g., as an injectable, paste, gel or mesh) during the implantation procedure; (b) to the surface of the tissue (e.g., as an injectable, paste, gel, in situ forming gel or mesh) immediately prior to, or during, implantation of the hemodialysis access device; (c) to the surface of the hemodialysis access device and/or the tissue surrounding the implanted hemodialysis access device (e.g., as an injectable, paste, gel, in situ forming gel or mesh) immediately after the implantation of the hemodialysis access device; (d) by topical application of the drug combination (or individual component(s) thereof) or the composition comprising the drug combination (or individual component(s) thereof) into the anatomical space where the hemodialysis access device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the hemodialysis access device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

In addition to the anti-fibrosis drug combinations (or individual components thereof), subject compositions infiltrated into tissue adjacent to hemodialysis access devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin).

According to the one aspect, any anti-fibrosis drug combinations (or individual components thereof) or compositions that comprise drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to hemodialysis access devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions that comprise drug combinations (or individual components thereof) for prevention or inhibition of fibrosis in accordance with the present invention will depend on a variety of factors, including the type of formulation, the location of the treatment site, and the type of condition being treated. As hemodialysis access devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosing drug combinations (or individual components thereof) or compositions that comprise drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination of the anti-fibrosis drug combinations (or individual components thereof) or compositions that comprise drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Films and Meshes

In one aspect, anti-fibrosis drug combinations (or individual components thereof) or compositions comprising anti-fibrosis drug combinations (or individual components thereof) may be infiltrated into tissue adjacent to a film or mesh. Infiltration of the anti-fibrosis drug combinations (or individual components thereof) or compositions comprising anti-fibrosis drug combinations (or individual components thereof) into tissue adjacent to the film or mesh can minimize fibrosis (or scarring) in the vicinity of the implant and may reduce or prevent the formation of adhesions between the implant and the surrounding tissue and/or may inhibit or prevent infection in the vicinity of the implant site. In certain aspects, the film or mesh may be used as a drug-delivery vehicle (e.g., as a perivascular delivery device for the prevention of neointimal hyperplasia at an anastomotic site).

Representative examples of films and meshes that may benefit from having the subject compositions infiltrated into adjacent tissue are provided above in conjunction with the coating of medical devices. Numerous agents or compositions for use with films and meshes have been described above which may be infiltrated into the tissue adjacent to the device (preferably near the device-tissue interface).

A variety of polymeric compositions have been described that may be used in conjunction with the films and meshes of the invention. Such compositions may be in the form of, for example, gels, sprays, liquids, and pastes, or may be polymerized from monomeric or prepolymeric constituents in situ. For example, the composition may be a polymeric tissue coating which is formed by applying a polymerization initiator to the tissue and then covering it with a water-soluble macromer that is polymerizable using free radical initiators under the influence of UV light. See, e.g., U.S. Pat. Nos. 6,177,095 and 6,083,524. The composition may be an aqueous composition including a surfactant, pentoxifylline and a polyoxyalkylene polyether. See, e.g., U.S. Pat. No. 6,399,624. The composition may be a hydrogel-forming, self-solvating, absorbable polyester copolymers capable of selective, segmental association into compliant hydrogels mass upon contact with an aqueous environment. See, e.g., U.S. Pat. No. 5,612,052. The composition may be composed of fluent pre-polymeric material that is emitted to the tissue surface and then exposed to activating energy in situ to initiate conversion of the applied material to non-fluent polymeric form. See, e.g., U.S. Pat. Nos. 6,004,547 and 5,612,050. The composition may be composed of a gas mixture of oxygen present in a volume ratio of 1 to 20%. See, e.g., U.S. Pat. No. 6,428,500. The composition may be composed of an anionic polymer having an acid sulfate and sulfur content greater than 5% which acts to inhibit monocyte or macrophage invasion. See, e.g., U.S. Pat. No. 6,417,173. The composition may be composed of a non-gelling polyoxyalkylene composition with or without a therapeutic agent. See, e.g., U.S. Pat. No. 6,436,425. The composition may be coated onto tissue surfaces and may be composed of an aqueous solution of a hydrophilic, polymeric material (e.g., polypeptides or polysaccharide) having greater than 50,000 molecular weight and a concentration range of 0.01% to 15% by weight. See, e.g., U.S. Pat. No. 6,464,970.

Other representative examples of polymeric compositions which may be infiltrated into tissue adjacent to the film or mesh include poly(ethylene glycol)-based systems, hyaluronic acid and crosslinked hyaluronic acid compositions. These compositions can be applied as the final composition or they can be applied as materials that form crosslinked gel in situ.

Other compositions that can be used in conjunction with films and meshes, include, but are not limited to: (a) sprayable PEG-containing formulations such as COSEAL, SPRAYGEL, FOCALSEAL or DURASEAL; (b) hyaluronic acid-containing formulations such as RESTYLANE, HYLAFORM, PERLANE, SYNVISC, SEPRAFILM, SEPRACOAT, INTERGEL, (c) polymeric gels such as REPEL or FLOWGEL, (d) dextran sulfate gels such as the ADCON range of products, (e) lipid based compositions such as ADSURF (Brittania Pharmaceuticals).

The film or mesh (or device comprising the film or mesh) may be made sterile either by preparing them under aseptic environment and/or they may be terminally sterilized using methods known in the art, such as gamma radiation or electron beam sterilization methods or a combination of both of these methods.

Films and meshes may be applied to any bodily conduit or any tissue that may be prone to the development of fibrosis or intimal hyperplasia. Prior to implantation, the film or mesh may be trimmed or cut from a sheet of bulk material to match the configuration of the widened foramen, canal, or dissection region, or at a minimum, to overlay the exposed tissue area. The film or mesh may be bent or shaped to match the particular configuration of the placement region. The film or mesh may also be rolled in a cuff shape or cylindrical shape and placed around the exterior periphery of the desired tissue. The film or mesh may be provided in a relatively large bulk sheet and then cut into shapes to mold the particular structure and surface topography of the tissue or device to be wrapped. Alternatively, the film or mesh may be pre-shaped into one or more patterns for subsequent use. The films and meshes may be typically rectangular in shape and be placed at the desired location within the surgical site by direct surgical placement or by endoscopic techniques. The film or mesh may be secured into place by wrapping it onto itself (i.e., self-adhesive), or by securing it with sutures, staples, sealant, and the like. Alternatively, the film or mesh may adhere readily to tissue and therefore, additional securing mechanisms may not be required.

The films or meshes of the invention may be used for a variety of indications, including, without limitation: (a) prevention of surgical adhesions between tissues following surgery (e.g., gynecologic surgery, vasovasostomy, hernia repair, nerve root decompression surgery and laminectomy); (b) prevention of hypertrophic scars or keloids (e.g., resulting from tissue burns or other wounds); (c) prevention of intimal hyperplasia and/or restenosis (e.g., resulting from insertion of vascular grafts or hemodialysis access devices); (d) may be used in affiliation with devices and implants that lead to scarring as described herein (e.g., as a sleeve or mesh around a breast implant to reduce or inhibit scarring); (e) prevention of infection (e.g., resulting from tissue bums, surgery or other wounds); or (f) may be used in affiliate with devices and implants that lead to infection as described herein.

In one embodiment, films or meshes may be used to prevent adhesions that occur between tissues following surgery, injury or disease. Adhesion formation, a complex process in which bodily tissues that are normally separate grow together, occurs most commonly as a result of surgical intervention and/or trauma. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation can be inhibited. Adhesion formation or unwanted scar tissue accumulation and encapsulation complicates a variety of surgical procedures and virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule which compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies, infections (fungal, mycobacterium). Surgical procedures that may lead to surgical adhesions may include cardiac, spinal, neurologic, pleural, thoracic and gynecologic surgeries. However, adhesions may also develop as a result of other processes, including, but not limited to, non-surgical mechanical injury, ischemia, hemorrhage, radiation treatment, infection-related inflammation, pelvic inflammatory disease and/or foreign body reaction. This abnormal scarring interferes with normal physiological functioning and, in come cases, can force and/or interfere with follow-up, corrective or other surgical operations. For example, these post-operative surgical adhesions occur in 60 to 90% of patients undergoing major gynecologic surgery and represent one of the most common causes of intestinal obstruction in the industrialized world. These adhesions are a major cause of failed surgical therapy and are the leading cause of bowel obstruction and infertility. Other adhesion-treated complications include chronic pelvic pain, urethral obstruction and voiding dysfunction.

Currently, preventative therapies, administered 4 to 5 days following surgery, are used to inhibit adhesion formation. Various modes of adhesion prevention have been examined, including (1) prevention of fibrin deposition, (2) reduction of local tissue inflammation, and (3) removal of fibrin deposits. Fibrin deposition is prevented through the use of physical adhesion barriers that are either mechanical or comprised of viscous solutions. Although many investigators are utilizing adhesion prevention barriers, a number of technical difficulties exist.

In one aspect, the present invention provides films and meshes having anti-fibrosis drug combinations (or individual components thereof) or compositions comprising anti-fibrosis drug combinations (or individual components thereof) infiltrated into adjacent tissue for use as surgical adhesion barriers.

In one aspect, films and meshes having anti-fibrosis drug combinations (or individual components thereof) or compositions comprising anti-fibrosis drug combinations (or individual components thereof) infiltrated into adjacent tissue may be used to prevent surgical adhesions in the epidural and dural tissue which is a factor contributing to failed back surgeries and complications associated with spinal injuries (e.g., compression and crush injuries). Scar formation within dura and around nerve roots has been implicated in rendering subsequent spine operations technically more difficult. To gain access to the spinal foramen during back surgeries, vertebral bone tissue is often disrupted. Back surgeries, such as laminectomies and diskectomies, often leave the spinal dura exposed and unprotected. As a result, scar tissue frequently forms between the dura and the surrounding tissue. This scar is formed from the damaged erector spinae muscles that overlay the laminectomy site. This results in adhesion development between the muscle tissue and the fragile dura, thereby, reducing mobility of the spine and nerve roots which leads to pain and slow post-operative recovery. To circumvent adhesion development, a scar-reducing barrier may be inserted between the dural sleeve and the paravertebral musculature post-laminotomy. This reduces cellular and vascular invasion into the epidural space from the overlying muscle and exposed cancellous bone and thus, reduces the complications associated with the canal housing the spinal chord and/or nerve roots.

In another aspect, films and meshes having anti-fibrosis drug combinations (or individual components thereof) or compositions comprising anti-fibrosis drug combinations (or individual components thereof) infiltrated into adjacent tissue may be used to prevent the fibrosis from occurring between a hernia repair mesh and the surrounding tissue. Hernias are abnormal protrusions (outpouchings) of an organ or other body structure through a defect or natural opening in a covering membrane, muscle or bone. Hernias themselves are not dangerous, but can become extremely problematic if they become incarcerated. Surgical prostheses used in hernia repair (referred to herein as “hernia meshes”) include prosthetic mesh-or gauze-like materials, which support the repaired hernia or other body structures during the healing process. Hernias are often repaired surgically to prevent complications. Conditions in which a hernia mesh may need to be used include, without limitation, the repair of inguinal (i.e., groin), umbilical, ventral, femoral, abdominal, diaphragmatic, epigastric, gastroesophageal, hiatal, intermuscular, mesenteric, paraperitoneal, rectovaginal, rectocecal, uterine, and vesical hernias. Hernia repair typically involves returning the viscera to its normal location and the defect in the wall is primarily closed with sutures, but for bigger gaps, a mesh is placed over the defect to close the hernia opening. Infiltration of the subject composition comprising an anti-scarring agent into tissue adjacent to a hernia repair mesh may reduce or prevent fibrosis proximate to the implanted hernia mesh, thereby minimizing the possibility of adhesions between the abdominal wall or other tissues and the mesh itself, and reducing further complications and abdominal pain.

In yet another aspect, films or meshes having anti-fibrosis drug combinations (or individual components thereof) or compositions comprising anti-fibrosis drug combinations (or individual components thereof) infiltrated into adjacent tissue may be used to prevent hypertrophic scars or keloids (e.g., resulting from tissue burns or other wounds). Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including bums, open wounds, and infected wounds. With hypertrophic scars, some degree of maturation occurs and gradual improvement occurs. In the case of keloids however, an actual tumor is produced which can become quite large. Spontaneous improvement in such cases rarely occurs. A film or mesh having the subject composition comprising an anti-scarring agent infiltrated into adjacent tissue may be placed in contact with a wound or burn site in order to prevent formation of hypertrophic scar or keloids.

In yet another aspect, films and meshes having anti-fibrosis drug combinations (or individual components thereof) or compositions comprising anti-fibrosis drug combinations (or individual components thereof) infiltrated into adjacent tissue are provided that may be used for delivering an anti-scarring drug combination (or individual component(s) thereof) to an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, Eustachian tubes, the external auditory mayal, vas deferens and fallopian tubes. Examples of cavities include the abdominal cavity, the buccal cavity, the peritoneal cavity, the pericardial cavity, the pelvic cavity, perivisceral cavity, pleural cavity and uterine cavity.

Examples of conditions that may be treated or prevented with films and meshes having anti-fibrosis drug combinations (or individual components thereof) or compositions comprising anti-fibrosis drug combinations (or individual components thereof) infiltrated into adjacent tissue include iatrogenic complications of arterial and venous catheterization, complications of vascular dissection, complications of gastrointestinal passageway rupture and dissection, restonotic complications associated with vascular surgery (e.g., bypass surgery), and intimal hyperplasia.

In one aspect, anti-fibrosis drug combinations (or individual components thereof) or compositions comprising anti-fibrosis drug combinations (or individual components thereof) may be delivered from the subject composition infiltrated into tissue adjacent to a film or mesh to the external walls of body passageways or cavities for the purpose of preventing and/or reducing a proliferative biological response that may obstruct or hinder the optimal functioning of the passageway or cavity, including, for example, iatrogenic complications of arterial and venous catheterization, aortic dissection, cardiac rupture, aneurysm, cardiac valve dehiscence, graft placement (e.g., A-V-bypass, peripheral bypass, CABG), fistula formation, passageway rupture and surgical wound repair.

The films or meshes may be used in the form of a perivascular wrap to prevent restenosis at anastomotic sites resulting from insertion of vascular grafts or hemodialysis access devices. In this case, perivascular wraps having anti-fibrosis drug combinations (or individual components thereof) or compositions comprising anti-fibrosis drug combinations (or individual components thereof) infiltrated into adjacent tissue may be used in conjunction with a vascular graft to inhibit scarring at an anastomotic site. These films or meshes may be placed or wrapped in a perivascular (periadventitial) manner around the outside of the anastomosis at the time of surgery. Film and mesh implants having anti-fibrosis drug combinations (or individual components thereof) or compositions comprising anti-fibrosis drug combinations (or individual components thereof) infiltrated into adjacent tissue may be used with synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts).

In order to further the understanding of such conditions, representative complications leading to compromised body passageway or cavity integrity are discussed in more detail below.

In one aspect, anti-fibrosis drug combinations (or individual components thereof) or compositions comprising anti-fibrosis drug combinations (or individual components thereof) may be infiltrated into tissue adjacent to a coronary artery bypass graft (“CABG”).

In one aspect, anti-fibrosis drug combinations (or individual components thereof) or compositions comprising anti-fibrosis drug combinations (or individual components thereof) may be infiltrated into tissue adjacent to a peripheral bypass surgery site.

In one aspect, anti-fibrosis drug combinations (or individual components thereof) or compositions comprising anti-fibrosis drug combinations (or individual components thereof) may be infiltrated into tissue adjacent to an arterio-venous fistula.

In one aspect, anti-fibrosis drug combinations (or individual components thereof) or compositions comprising anti-fibrosis drug combinations (or individual components thereof) may be infiltrated into tissue adjacent to a peripheral bypass surgery site.

In one aspect, anti-fibrosis drug combinations (or individual components thereof) or compositions comprising anti-fibrosis drug combinations (or individual components thereof) may be infiltrated into tissue adjacent to an anastomotic closure device.

In one aspect, anti-fibrosis drug combinations (or individual components thereof) or compositions comprising anti-fibrosis drug combinations (or individual components thereof) may be infiltrated into tissue adjacent to a transplant surgery site.

According to the one aspect, any anti-fibrosis drug combinations (or individual components thereof) or compositions comprising anti-fibrosis drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to films and meshes may be adapted to contain and/or release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for prevention or inhibition of fibrosis in accordance with the present invention will depend on a variety of factors, including the type of formulation, the location of the treatment site, and the type of condition being treated. As films and meshes are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Glaucoma Drainage Devices

In one aspect, the anti-fibrosis drug combinations (or individual components thereof) or compositions comprising anti-fibrosis drug combinations (or individual components thereof) may be infiltrated into tissue adjacent to a glaucoma drainage device.

Representative examples glaucoma drainage devides that may benefit from having anti-fibrosis drug combinations (or individual components thereof) or compositions comprising anti-fibrosis drug combinations (or individual components thereof) infiltrated into adjacent tissue are provided above in conjunction with the coating of medical devices. Numerous agents or compositions for use with glaucoma drainage devices have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted glaucoma drainage devices by applying the composition directly and/or indirectly into and/or onto (a) tissue adjacent to the glaucoma drainage device; (b) the vicinity of the glaucoma drainage device-tissue interface; (c) the region around the glaucoma drainage device; and (d) tissue surrounding the glaucoma drainage device.

Methods for infiltrating the drug combination (or individual component(s) thereof) or the composition comprising the drug combination (or individual component(s) thereof) into tissue adjacent to a glaucoma drainage device include delivering the drug combination (or individual component(s) thereof) or the composition comprising the drug combination (or individual component(s) thereof): (a) to the glaucoma drainage device surface (e.g., as an injectable, paste, gel or mesh) during the implantation procedure; (b) to the surface of the tissue (e.g., as an injectable, paste, gel, in situ forming gel or mesh) immediately prior to, or during, implantation of the glaucoma drainage device; (c) to the surface of the glaucoma drainage device and/or the tissue surrounding the implanted glaucoma drainage device (e.g., as an injectable, paste, gel, in situ forming gel or mesh) immediately after the implantation of the glaucoma drainage device; (d) by topical application of the drug combination (or individual component(s) thereof) or the composition comprising the drug combination (or individual component(s) thereof) into the anatomical space where the glaucoma drainage device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the glaucoma drainage device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

In one aspect, the methods above can be used to infiltrate the anti-fibrosis drug combinations (or individual components thereof) or compositions comprising anti-fibrosis drug combinations (or individual components thereof) into tissue adjacent to all or portions of the plate of the device.

In another aspect, the methods above can be used to infiltrate the anti-fibrosis drug combinations (or individual components thereof) or compositions comprising anti-fibrosis drug combinations (or individual components thereof) into tissue adjacent to all or portions of the tube of the device.

In yet another aspect, the methods above can be used to infiltrate anti-fibrosis drug combinations (or individual components thereof) or compositions comprising anti-fibrosis drug combinations (or individual components thereof) into tissue adjacent to all or potions of both the plate and the tube of the device.

According to the present invention, any anti-fibrosis drug combinations (or individual components thereof) or compositions comprising anti-fibrosis drug combinations (or individual components thereof) described above can be utilized in the practice of the present invention. In one aspect of the invention, the anti-fibrosis drug combinations (or individual components thereof) or compositions comprising anti-fibrosis drug combinations (or individual components thereof) infiltrated into tissue adjacent to glaucoma drainage devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for prevention or inhibition of fibrosis in accordance with the present invention will depend on a variety of factors, including the type of formulation, the location of the treatment site, and the type of condition being treated. As glaucoma drainage devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with anti-fibrosis drug combinations (or individual components thereof) or compositions comprising anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti- infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 g-1 mg, or about 1 mg to 10 mg, or about 10 mg -100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 g/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Prosthetic Heart Valves

In one aspect, anti-fibrosis drug combinations (or individual components thereof) or compositions comprising anti-fibrosis drug combinations (or individual components thereof) may be infiltrated into tissue adjacent to a prosthetic heart valve.

Representative examples of prosthetic heart valves that may benefit from having the anti-fibrosis drug combinations (or individual components thereof) or compositions comprising anti-fibrosis drug combinations (or individual components thereof) infiltrated into adjacent tissue are provided above in conjunction with the coating of medical devices. Numerous agents or compositions for use with prosthetic heart valves have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted prosthetic heart valves by applying the composition directly and/or indirectly into and/or onto (a) tissue adjacent to the prosthetic heart valve; (b) the vicinity of the prosthetic heart valve-tissue interface; (c) the region around the prosthetic heart valve; and (d) tissue surrounding the prosthetic heart valve.

Methods for infiltrating the anti-fibrosis drug combinations (or individual components thereof) or compositions comprising anti-fibrosis drug combinations (or individual components thereof) into tissue adjacent to a prosthetic heart valve include delivering the drug combination (or individual component(s) thereof) or the composition comprising the drug combination (or individual component(s) thereof): (a) to the prosthetic heart valve surface (e.g., as an injectable, paste, gel or mesh) during the implantation procedure; (b) to the surface of the tissue (e.g., as an injectable, paste, gel, in situ forming gel or mesh) immediately prior to, or during, implantation of the prosthetic heart valve; (c) to the surface of the prosthetic heart valve and/or the tissue surrounding the implanted prosthetic heart valve (e.g., as an injectable, paste, gel, in situ forming gel or mesh) immediately after the implantation of the prosthetic heart valve; (d) by topical application of the drug combination (or individual component(s) thereof) or the composition comprising the drug combination (or individual component(s) thereof) into the anatomical space where the prosthetic heart valve may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the prosthetic heart valve as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

In some aspects, the anti-fibrosis drug combinations (or individual components thereof) or compositions comprising anti-fibrosis drug combinations (or individual components thereof) may be infiltrated into tissue adjacent to: (a) the surface of the annular ring (particularly mechanical valves); (b) the surface of the valve leaflets (particularly bioprosthetic valves); and/or (c) any combination of the aforementioned.

According to one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to prosthetic heart valves may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As prosthetic heart valves are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Penile Implants

In one aspect, anti-fibrosis drug combinations (or individual components thereof) or compositions comprising anti-fibrosis drug combinations (or individual components thereof) may be infiltrated into tissue adjacent to a penile implant device.

Representative examples of penile implants that may benefit from having the subject compositions infiltrated into adjacent tissue are provided above in conjunction with the coating of medical devices. Numerous agents or compositions for use with penile implants have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted penile implants by applying the composition directly and/or indirectly into and/or onto (a) tissue adjacent to the penile implant; (b) the vicinity of the penile implant-tissue interface; (c) the region around the penile implant; and (d) tissue surrounding the penile implant.

Methods for infiltrating the subject compositions into tissue adjacent to a penile implant include delivering the drug combination (or individual component(s) thereof) or the composition comprising the drug combination (or individual component(s) thereof): (a) to the penile implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the penile implant; (c) to the surface of the penile implant and/or the tissue surrounding the implanted penile implant (e.g., as an injectable, paste, gel, in situ forming gel or mesh) immediately after the implantation of the penile implant; (d) by topical application of the drug combination (or individual component(s) thereof) or the composition comprising the drug combination (or individual component(s) thereof) into the anatomical space where the penile implant may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component9s) thereof) may be delivered into the region where the device may be inserted); (e) via percutaneous injection into the tissue surrounding the penile implant as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

The placement of penile implants can be complicated by infection (usually in the first 6 months after surgery) with Coagulase Negative Staphylococci (including Staphylococcus epidermidis), Staphylococcus aureus, Pseudomonas aeruginosa, Enterococci, Serratia and Candida. Infection is characterized by fever, erythema, induration and purulent drainage from the operative site. The usual route of infection is through the incision at the time of surgery and up to 3% of penile implants become infected despite the best sterile surgical technique. To help combat this, intraoperative irrigation with antibiotic solutions is often employed.

Infiltrating into the tissue adjacent to the penile implant a composition containing an anti-infective agent can allow bacteriocidal drug levels to be achieved locally, thus reducing the incidence of bacterial colonization (and subsequent development of local infection and device failure), while producing negligible systemic exposure to the drugs.

According to the one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to penile implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for prevention or inhibition of fibrosis in accordance with the present invention will depend on a variety of factors, including the type of formulation, the location of the treatment site, and the type of condition being treated. As penile implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 g-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Endotracheal and Tracheostomy Tubes

In one aspect, anti-fibrosis drug combinations (or individual components thereof) or compositions comprising anti-fibrosis drug combinations (or individual components thereof) may be infiltrated into tissue adjacent to endotracheal and tracheostomy tube devices. Association of an anti-scarring agent with an endotracheal or a tracheostomy tube (e.g., chest tube), or adjacent tissue, may be used to prevent stenosis and/or infection of the artificial airway.

Representative examples of endotracheal and tracheostomy tubes that may benefit from having the anti-fibrosis drug combinations (or individual components thereof) or compositions comprising anti-fibrosis drug combinations (or individual components thereof) infiltrated into adjacent tissue are provided above in conjunction with the coating of medical devices. Numerous agents or compositions for use with endotracheal and tracheostomy tubes have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted endotracheal and tracheostomy tube devices by applying the composition directly and/or indirectly into and/or onto (a) tissue adjacent to the endotracheal or tracheostomy tube device; (b) the vicinity of the endotracheal or tracheostomy tube device-tissue interface; (c) the region around the endotracheal or tracheostomy tube device; and (d) tissue surrounding the endotracheal or tracheostomy tube device.

Methods for infiltrating the subject compositions into tissue adjacent to endotracheal or tracheostomy tube devices include delivering the drug combination (or individual component(s) thereof) or the composition comprising the drug combination (or individual component(s) thereof): (a) to the endotracheal or tracheostomy tube device surface (e.g., as an injectable, paste, gel or mesh) during the implantation procedure; (b) to the surface of the tissue (e.g., as an injectable, paste, gel, in situ forming gel or mesh) immediately prior to, or during, implantation of the endotracheal or tracheostomy tube device; (c) to the surface of the endotracheal or tracheostomy tube device and/or the tissue surrounding the implanted endotracheal or tracheostomy tube device (e.g., as an injectable, paste, gel, in situ forming gel or mesh) immediately after the implantation of the endotracheal or tracheostomy tube device; (d) by topical application of the drug combination (or individual component(s) thereof) or the composition comprising the drug combination (or individual component(s) thereof) into the anatomical space where the endotracheal or tracheostomy tube device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the endotracheal or tracheostomy tube device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or compositions comprising anti-fibrosis drug combinations (or individual components thereof) described above may be utilized in the practice of the present invention. In one aspect of the invention, the drug combination (or individual component(s) thereof) or the composition comprising the drug combination (or individual component(s) thereof) infiltrated into tissue adjacent to endotracheal and tracheostomy tube devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As endotracheal and tracheostomy tube devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Peritoneal Dialysis Catheters

In one aspect, anti-fibrosis drug combinations (or individual components thereof) or compositions comprising anti-fibrosis drug combinations (or individual components thereof) may be infiltrated into tissue adjacent to a peritoneal dialysis catheter or a peritoneal implant for drug delivery.

Representative examples of peritoneal dialysis catheters that may benefit from having the anti-fibrosis drug combinations (or individual components thereof) or compositions comprising anti-fibrosis drug combinations (or individual components thereof) infiltrated into adjacent tissue are provided above in conjunction with the coating of medical devices. Numerous agents or compositions for use with peritoneal dialysis catheters have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted peritoneal access catheters and implants by applying the composition directly and/or indirectly into and/or onto (a) tissue adjacent to the peritoneal access catheter or implant; (b) the vicinity of the peritoneal access catheter or implant-tissue interface; (c) the region around the peritoneal access catheter or implant; and (d) tissue surrounding the peritoneal access catheter or implant.

Methods for infiltrating the anti-fibrosis drug combinations (or individual components thereof) or compositions comprising anti-fibrosis drug combinations (or individual components thereof) into tissue adjacent to a peritoneal access catheter or implant include delivering the drug combination (or individual component(s) thereof) or the composition comprising the drug combination (or individual component(s) thereof): (a) to the peritoneal access catheter or implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the peritoneal access catheter or implant; (c) to the surface of the peritoneal access catheter or implant and/or the tissue surrounding the implanted peritoneal access catheter or implant (e.g., as an injectable, paste, gel, in situ forming gel or mesh) immediately after the implantation of the peritoneal access catheter or implant; (d) by topical application of the drug combination (or individual component(s) thereof) or the composition comprising the drug combination (or individual component(s) thereof) into the anatomical space where the peritoneal access catheter or implant may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the peritoneal access catheter or implant as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to peritoneal dialysis implants and catheters may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As peritoneal dialysis implants and catheters are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising the anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm-1 μg/mm², or about 1 μg/mm²-10 μg/mm², or about 10 μg/mm²-100 μg/mm², or about 100 μg/mm^{2 to} 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Central Nervous System Shunts and Pressure Monitoring Devices

In one aspect, anti-fibrosis drug combinations (or individual components thereof) or compositions comprising anti-fibrosis drug combinations (or individual components thereof) may be infiltrated into tissue adjacent to a central nervous system (CNS) device, such as a CNS shunt or a pressure monitoring device. CNS devices having the anti-fibrosis drug combinations (or individual components thereof) or compositions comprising anti-fibrosis drug combinations (or individual components thereof) infiltrated into adjacent tissue are capable of preventing stenosis and obstruction of the device leading to hydrocephalus and increased intercranial pressure.

Representative examples of CNS and pressure monitoring devices that may benefit from having the anti-fibrosis drug combinations (or individual components thereof) or compositions comprising anti-fibrosis drug combinations (or individual components thereof) infiltrated into adjacent tissue are provided above in conjunction with the coating of medical devices. Numerous agents or compositions for use with CNS and pressure monitoring devices have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted CNS devices by applying the composition directly and/or indirectly into and/or onto (a) tissue adjacent to the CNS device; (b) the vicinity of the CNS device-tissue interface; (c) the region around the CNS device; and (d) tissue surrounding the CNS device.

Methods for infiltrating the subject compositions into tissue adjacent to a CNS device include delivering the drug combination (or individual component(s) thereof) or the composition comprising the drug combination (or individual component(s) thereof): (a) to the CNS device surface (e.g., as an injectable, paste, gel or mesh) during the implantation procedure; (b) to the surface of the tissue (e.g., as an injectable, paste, gel, in situ forming gel or mesh) immediately prior to, or during, implantation of the CNS device; (c) to the surface of the CNS device and/or the tissue surrounding the implanted CNS device (e.g., as an injectable, paste, gel, in situ forming gel or mesh) immediately after the implantation of the CNS device; (d) by topical application of the anti-fibrosis drug combination (or individual component(s) thereof) or the composition that comprises the anti-fibrosis drug combination (or individual component(s) thereof) into the anatomical space where the CNS device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the CNS device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to CNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As CNS devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising the anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used in combination with anti-fibrosis drug combinations (or individual components thereof) or compositions comprising anti-fibrosis drug combinations (or individual components there under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Inferior Vena Cava Filters

In one aspect, anti-fibrosis drug combinations (or individual components thereof) or compositions comprising anti-fibrosis drug combinations (or individual components thereof) may be infiltrated into tissue adjacent to an inferior vena cava filter device.

Representative examples of inferior vena cava filters that may benefit from having the subject compositions infiltrated into adjacent tissue are provided above in conjunction with the coating of medical devices. Numerous agents or compositions for use with inferior vena cava filters have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted inferior vena cava filter devices by applying the composition directly and/or indirectly into and/or onto (a) tissue adjacent to the inferior vena cava filter device; (b) the vicinity of the inferior vena cava filter device-tissue interface; (c) the region around the inferior vena cava filter device; and (d) tissue surrounding the inferior vena cava filter device.

Methods for infiltrating the subject compositions into tissue adjacent to an inferior vena cava filter device include delivering the drug combination (or individual component(s) thereof) or the composition comprising the drug combination (or individual component(s) thereof): (a) to the inferior vena cava filter device surface (e.g., as an injectable, paste, gel or mesh) during the implantation procedure; (b) to the surface of the tissue (e.g., as an injectable, paste, gel, in situ forming gel or mesh) immediately prior to, or during, implantation of the inferior vena cava filter device; (c) to the surface of the inferior vena cava filter device and/or the tissue surrounding the implanted inferior vena cava filter device (e.g., as an injectable, paste, gel, in situ forming gel or mesh) immediately after the implantation of the inferior vena cava filter device; (d) by topical application of the anti-fibrosis drug combination (or individual component(s) thereof) or the composition comprising the anti-fibrosis drug combination (or individual component(s) thereof) into the anatomical space where the inferior vena cava filter device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the inferior vena cava filter device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to vena cava filters (e.g., inferior vena cava filters) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As inferior vena cava filter devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with the anti-fibrosis drug combinations (or individual components thereof) or compositions comprising anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Gastrointestinal Devices

In one aspect, anti-fibrosis drug combinations (or individual components thereof) or compositions comprising anti-fibrosis drug combinations (or individual components thereof) may be infiltrated into tissue adjacent to a gastrointestinal (GI) device.

Representative examples of GI devices that may benefit from having the anti-fibrosis drug combinations (or individual components thereof) or compositions comprising anti-fibrosis drug combinations (or individual components thereof) infiltrated into adjacent tissue are provided above in conjunction with the coating of medical devices. Numerous agents or compositions for use with GI devices have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) may be infiltrated around implanted GI devices by applying the composition directly and/or indirectly into and/or onto (a) tissue adjacent to the GI device; (b) the vicinity of the GI device-tissue interface; (c) the region around the GI device; and (d) tissue surrounding the GI device.

Methods for infiltrating the anti-fibrosis drug combination (or individual component(s) thereof) or the composition comprising the anti-fibrosis drug combination (or individual component(s) thereof) into tissue adjacent to a GI device include delivering the drug combination (or individual component(s) thereof) or the composition comprising the drug combination (or individual component(s) thereof): (a) to the GI device surface (e.g., as an injectable, paste, gel or mesh) during the implantation procedure; (b) to the surface of the tissue (e.g., as an injectable, paste, gel, in situ forming gel or mesh) immediately prior to, or during, implantation of the GI device; (c) to the surface of the GI device and/or the tissue surrounding the implanted GI device (e.g., as an injectable, paste, gel, in situ forming gel or mesh) immediately after the implantation of the GI device; (d) by topical application of the anti-fibrosis drug combination (or individual component(s) thereof) or the composition comprising the anti-fibrosis drug combination (or individual component(s) thereof) into the anatomical space where the GI device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the GI device as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

According to one aspect, any anti-fibrosis drug combinations (or individual components thereof) or compositions comprising anti-fibrosis drug combinations (or individual components thereof) described above may be utilized in the practice of the present invention. In one aspect of the invention, the anti-fibrosis drug combination (or individual component(s) thereof) or the composition comprising the anti-fibrosis drug combination (or individual component(s) thereof) infiltrated into tissue adjacent to GI devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As GI devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Central Venous Catheters

In one aspect, anti-fibrosis drug combinations (or individual components thereof) or compositions comprising anti-fibrosis drug combinations (or individual components thereof) may be infiltrated into tissue adjacent to a central venous catheter (CVC) device such that the overgrowth of granulation tissue is inhibited or reduced.

Representative examples genital-urinary (GU) stents that may benefit from having the subject compositions infiltrated into adjacent tissue are provided above in conjunction with the coating of medical devices. Numerous agents or compositions for use with tracheal and bronchial have been described above which may be infiltrated into the tissue adjacent to the device (preferably near the device-tissue interface). Polymeric compositions may be infiltrated around implanted CVC devices by applying the composition directly and/or indirectly into and/or onto (a) tissue adjacent to the CVC device; (b) the vicinity of the CVC device-tissue interface; (c) the region around the CVC device; and (d) tissue surrounding the CVC device.

Methods for infiltrating the anti-fibrosis drug combination (or individual component(s) thereof) or the composition comprising the anti-fibrosis drug combination (or individual component(s) thereof) into tissue adjacent to a CVC device include delivering the anti-fibrosis drug combination (or individual component(s) thereof) or the composition comprising the anti-fibrosis drug combination (or individual component(s) thereof): (a) to the CVC device surface (e.g., as an injectable, paste, gel or mesh) during the implantation procedure; (b) to the surface of the tissue (e.g., as an injectable, paste, gel, in situ forming gel or mesh) immediately prior to, or during, implantation of the CVC device; (c) to the surface of the CVC device and/or the tissue surrounding the implanted CVC device (e.g., as an injectable, paste, gel, in situ forming gel or mesh) immediately after the implantation of the CVC device; (d) by topical application of the anti-fibrosis drug combination (or individual component(s) thereof) or the composition comprising the anti-fibrosis drug combination (or individual component(s) thereof) into the anatomical space where the CVC device may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the CVC device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device.

In some aspects, the anti-fibrosis drug combinations (or individual components thereof) or compositions comprising anti-fibrosis drug combinations (or individual components thereof) may infiltrated into tissue adjacent to: (a) the exterior surface of the intravascular portion of the CVC device and/or the segment of the CVC device that traverses the skin; (b) exterior surface of the intravascular portion of the CVC device and/or the segment of the CVC device that traverses the skin, where the interior and/or exterior of the CVC device is coated with a composition comprising a therapeutic agent (e.g., an anti-infective agent); (c) the surface of, a subcutaneous “cuff” around the CVC device; (d) other surfaces of the CVC device; and (e) any combination of the aforementioned.

According to one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to CVC devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As CVC devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with anti-fibrosis drug combinations (or individual components thereof) or compositions comprising anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used in combination with anti-fibrosis drug combinations (or individual components thereof) or compositions comprising anti-fibrosis drug combinations (or individual components thereof), should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 g/mm², or about 10 g/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Ventricular Assist Devices

In one aspect, anti-fibrosis drug combinations (or individual components thereof) or compositions comprising anti-fibrosis drug combinations (or individual components thereof) may be infiltrated into tissue adjacent to a ventricular assist device (VAD).

Representative examples of VAD's that may benefit from having the subject compositions infiltrated into adjacent tissue are provided above in conjunction with the coating of medical devices. Numerous agents or compositions for use with VAD's have been described above which may be infiltrated into the tissue adjacent to the device (preferably near the device-tissue interface).

Anti-fibrosis drug combinations (or individual components thereof) or compositions comprising anti-fibrosis drug combinations (or individual components thereof) be infiltrated around implanted VADs by applying the composition directly and/or indirectly into and/or onto (a) tissue adjacent to the VAD; (b) the vicinity of the VAD-tissue interface; (c) the region around the VAD; and (d) tissue surrounding the VAD.

Methods for infiltrating the anti-fibrosis drug combination (or individual component(s) thereof) or the composition comprising the anti-fibrosis drug combination (or individual component(s) thereof) into tissue adjacent to a VAD include delivering the anti-fibrosis drug combination (or individual component(s) thereof) or the composition comprising the anti-fibrosis drug combination (or individual component(s) thereof): (a) to the VAD surface (e.g., as an injectable, paste, gel or mesh) during the implantation procedure; (b) to the surface of the tissue (e.g., as an injectable, paste, gel, in situ forming gel or mesh) immediately prior to, or during, implantation of the VAD; (c) to the surface of the VAD and/or the tissue surrounding the implanted VAD (e.g., as an injectable, paste, gel, in situ forming gel or mesh) immediately after the implantation of the VAD; (d) by topical application of the anti-fibrosis drug combination (or individual component(s) thereof) or the composition comprising the anti-fibrosis drug combination (or individual component(s) thereof) into the anatomical space where the VAD may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the VAD as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

According to the one aspect, any anti-fibrosis drug combinations (or individual components thereof) or compositions comprising anti-fibrosis drug combinations (or individual components thereof) described above may be utilized in the practice of the present invention. In one aspect of the invention, the subject compositions infiltrated into tissue adjacent to VADs (e.g., LVAD's) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As VADs are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm ²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with anti-fibrosis drug combinations (or individual components thereof) or compositions comprising anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 10 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Spinal Implants

In one aspect, anti-fibrosis drug combinations (or individual components thereof) or compositions comprising anti-fibrosis drug combinations (or individual components thereof) may be infiltrated into tissue adjacent to a spinal implant (e.g., a spinal prosthesis).

Representative examples of spinal implants that may benefit from having the subject compositions infiltrated into adjacent tissue are provided above in conjunction with the coating of medical devices. Numerous agents or compositions for use with spinal implants have been described above which may be infiltrated into the tissue adjacent to the device (preferably near the device-tissue interface). Infiltration of the subject compositions comprising a fibrosis-inhibiting agent and/or anti-infective agent into tissue adjacent to a spinal implant can minimize fibrosis (or scarring) in the vicinity of the implant and/or may reduce or prevent the formation of adhesions between the implant and the surrounding tissue and/or may inhibit or prevent infection in the vicinity of the implant.

Anti-fibrosis drug combinations (or individual components thereof) or compositions comprising anti-fibrosis drug combinations (or individual components thereof) may be infiltrated around implanted spinal implants by applying the composition directly and/or indirectly into and/or onto (a) tissue adjacent to the spinal implant; (b) the vicinity of the spinal implant-tissue interface; (c) the region around the spinal implant; and (d) tissue surrounding the spinal implant.

Methods for infiltrating the anti-fibrosis drug combination (or individual component(s) thereof) or the composition comprising the anti-fibrosis drug combination (or individual component(s) thereof) into tissue adjacent to a spinal implant include delivering the anti-fibrosis drug combination (or individual component(s) thereof) or the composition comprising the anti-fibrosis drug combination (or individual component(s) thereof): (a) to the spinal implant surface (e.g., as an injectable, paste, gel or mesh) during the implantation procedure; (b) to the surface of the tissue (e.g., as an injectable, paste, gel, in situ forming gel or mesh) immediately prior to, or during, implantation of the spinal implant; (c) to the surface of the spinal implant and/or the tissue surrounding the implanted spinal implant (e.g., as an injectable, paste, gel, in situ forming gel or mesh) immediately after the implantation of the spinal implant; (d) by topical application of the anti-fibrosis drug combination (or individual component(s) thereof) or the composition comprising the anti-fibrosis drug combination (or individual component(s) thereof) into the anatomical space where the spinal implant may be placed (particularly useful for this embodiment is the use of polymeric carriers which release the drug combination (or individual component(s) thereof) over a period ranging from several hours to several weeks—fluids, suspensions, emulsions, microemulsions, microspheres, pastes, gels, microparticulates, sprays, aerosols, solid implants and other formulations which release the drug combination (or individual component(s) thereof) may be delivered into the region where the device may be inserted); (e) via percutaneous injection into the tissue surrounding the spinal implant as a solution as an infusate or as a sustained release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device.

In one aspect, the anti-fibrosis drug combinations (or individual components thereof) or compositions comprising anti-fibrosis drug combinations (or individual components thereof) are infiltrated into the tissue adjacent to a spinal implant (e.g., an implantable cages or disc). In certain aspects, the spinal implant may be coated with (or adapted to contain) a fibrosis-inducing agent (e.g., silk or talc) on one part of the device and the anti-fibrosis drug combinations (or individual components thereof) or compositions comprising anti-fibrosis drug combinations (or individual components thereof) may be infiltrated into tissue adjacent to another part of the device. For example, the outer surface of the implant (e.g., a vertebral implant) may be coated with a fibrosis-inducing agent to improve adhesion between the device and the surrounding tissue, while the subject composition comprising an anti-fibrosis drug combination (or individual component(s) thereof) or a composition comprising an anti-fibrosis drug combination (or individual component(s) thereof) may be infiltrated into tissue adjacent to the interior of the device to minimize adhesion of tissue to the interior of the implant. Examples of fibrosis-inducing agents and methods of using fibrosis-inducing agents in combination with spinal implants are described in co-pending application entitled, “Medical Implants and Fibrosis-Inducing Agents,” filed Nov. 20, 2003 (U.S. Ser. No. 60/524,023) and Jun. 9, 2004 (U.S. Ser. No. 60/578,471). Additional examples of fibrosis-inducing agents include angiolytic agents (i.e., agents which cause leakage of vessels) such as EXHERIN from Adherex Technologies Inc. (Canada).

According to one aspect, any anti-fibrosis drug combinations (or individual components thereof) or compositions comprising anti-fibrosis drug combinations (or individual components thereof) described above can be utilized in the practice of the present invention. In one aspect of the invention, the subject compositions infiltrated into tissue adjacent to spinal implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting drug combinations for use in the present invention include the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis drug combinations (or individual components thereof) or compositions comprising anti-fibrosis drug combinations (or individual components thereof) for prevention or inhibition of fibrosis in accordance with the present invention will depend on a variety of factors, including the type of formulation, the location of the treatment site, and the type of condition being treated. As spinal implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination (or individual component(s) thereof) is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. It should be known that drugs within the combination may be released at different rates for different periods of time.

The exemplary anti-fibrosis drug combinations (or individual components thereof) or compositions comprising the anti-fibrosis drug combinations (or individual components thereof) should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent(s) are applied may be in the range of about 0.01 μg/mm²-1 μg/mm², or about 1 μg/mm²-1 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with anti-fibrosis drug combinations (or individual components thereof) or compositions comprising anti-fibrosis drug combinations (or individual components thereof) in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), (H) quinolones, as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used in combination with anti-fibrosis drug combinations (or individual components thereof) or compositions comprising anti-fibrosis drug combinations (or individual components thereof), should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10-6 about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, (e.g., methotrexate), quinolones, and/or podophylotoxins (e.g., etoposide) may be utilized to enhance the antibacterial activity of the composition.

The following examples are offered by way of illustration, and not by way of limitation.

### EXAMPLES

### Example 1

### Preparation of Drug Combination-Loaded Microspheres by Spray Drying

3.6 grams of methoxy poly(ethylene glycol 5000))-block-(poly (DL-lactide). (65:35 MePEG:PDLLA weight ratio) is dissolved in 200 ml methylene chloride. 100 mg of a drug combination, amoxapine and prednisolone, that is previously sieved through a 100 μm stainless steel sieve, is added and the resulting solution is spray dried. Inlet temperature 50° C., outlet temperature<39° C., aspirator 100%, flow rate 700 l/hr. The collected microspheres are dried under vacuum at room temperature overnight to produce uniform, spherical particles having size ranges of less than about 10 microns (typically about 0.5 to about 2 microns). This process for preparing drug combination-containing microspheres may be used for prepare microspheres containing other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), and albendazole and pentamidine, itraconazole and lovastatin.

### Example 2

### Drug Combination-Loaded Microspheres (<10 Micron) by the w/o/w Emulsion Process

100 ml of freshly prepared 10% polyvinyl alcohol (PVA) solution is added into a 600 ml beaker. The PVA solution is stirred at 2000 rpm for 30 minutes. Meanwhile, 40 mg of a drug combination, amoxapine and prednisolone, is added to a solution of 800 mg MePEG5000-PDLLA (65:35) in 20 ml dichloromethane. This mixture is stirred for 20 min. The resultant mixture is added dropwise to the PVA solution while stirring at 2000 rpm with a Fisher DYNA-MIX stirrer. After addition is complete, the solution is allowed to stir for an additional 45 minutes. The microsphere solution is transferred to several disposable graduated polypropylene conical centrifuge tubes, washed with deionized water, and centrifuged at 2600 rpm for 10 minutes. The aqueous layer is decanted and the washing, centrifuging and decanting is repeated 3 times. The combined, washed microspheres are freeze-dried and vacuum dried to remove any excess water. This process for preparing drug combination-containing microspheres may be used for preparing microspheres containing other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 3

### Drug Combination-Containing Microspheres (50-100 micron) by the w/o/w Emulsion Process

Microspheres having an average size of about 50-100 microns are prepared using a 1% PVA solution and 500 rpm stirring rate using the same procedure described in Example 2.

### Example 4

### Drug Combination Containing Micelles

MePEG2000 (4.1 g) and MePEG2000-PDLLA (60:40) (41 g) are combined in a vessel and heated to 75° C. with stirring. After the polymers are completely melted and mixed, the temperature was decreased to 55° C. A solution of a drug combination, amoxapine and prednisolone, in tetrahydrofuran (4.6 g/20 ml) is prepared and is poured into the polymer solution under constant stirring. Stirring is continued for an additional hour. The drug combination containing micelles are dried at 50° C. under vacuum to remove solvent. The resultant solid material is ground on a 2 mm mesh screen after cooling. This method for preparing drug combination-containing micelles may be used to prepare micelles containing other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 5

### Tetra Functional Poly (Ethylene Glycol) Succinimidyl Glutarate, (PEG-SG4), Non-Gelling Formulation

A 1 ml syringe (syringe 1) equipped with a luer-lock mixing connector is filled with PEG-SG4 (100 mg) (Sunbio, Inc., Orinda, Calif.). A 1 ml capped syringe (syringe 2) is filled with 0.25 ml of 6.3 mM HCl solution (pH 2.1). A 1 ml capped syringe (syringe 3) is filled with 0.25 ml 0.12 M monobasic sodium phosphate and 0.2 M sodium carbonate (pH 9.7) buffer. The solid contents of syringe 1 and the acidic solution of syringe 2 are mixed through a mixing connector by repeatedly transferring the contents from one syringe to the other. After complete mixing, the entire mixture is pushed into one of the syringes. The syringe containing the mixture then is attached to one inlet of an applicator (MICROMEDICS air assisted spray-applicator (Model SA-6105)). Syringe 3 containing the pH 9.7 solution is attached onto another inlet of the applicator. The formulation is applied to a tissue surface as specified by the applicator manufacturer.

### Example 6

### Gelling Formulation (Premix) I

A 1 ml syringe (syringe 1) equipped with a luer-lock mixing connector is filled with a mixture of PEG-SG4 (50mg) and PEG-SH4 (tetra functional poly (ethylene glycol) thiol) (50 mg) (Sunbio, Inc.) (referred to as “premix”). A 1 ml capped syringe (syringe 2) is filled with 0.25 ml of 6.3 mM HCI solution (pH 2. 1). A 1 ml capped syringe (syringe 3) is filled with 0.25 ml 0.12 M monobasic sodium phosphate and 0.2 M sodium carbonate (pH 9.7) buffer. The components are mixed and applied to a tissue surface using the procedure described in Example 5.

### Example 7

### Tetra Functional Poly (Ethylene Glycol) Amine, (PEG-N4) Gelling Formulation

A 1 ml syringe (syringe 1) equipped with a luer-lock mixing connector is filled with PEG-SG4 (50mg) and PEG-SH4 (tetra functional poly (ethylene glycol) thiol) (10, 25 or 40 mg). A 1 ml capped syringe (syringe 2) is filled with 0.25 ml of 6.3 mM HCI solution (pH 2.1). A 1 ml capped syringe (syringe 3) is filled with 0.25 ml 0.12 M monobasic sodium phosphate and 0.2 M sodium carbonate (pH 9.7) buffer. A 1 ml syringe (syringe 4) equipped with luer-lock mixing connector is filled with PEG-N4 (Sunbio, Inc.) (40, 25 or 10 mg) to make a mixture (50 mg total) of PEG-SH4 (in syringe 1) and PEG-N4 (in syringe 4). The contents of syringe 1 and syringe 2 are mixed through the mixing connector by repeatedly transferring the contents from one syringe to the other. After complete mixing, all of the formulation is pushed into one of the syringes which is then attached to one inlet of an applicator (MICROMEDICS air assisted spray-applicator (Model SA-6105)). Syringe 4 is attached to syringe 3 containing the pH 9.7 solution with a mixing connector. After complete mixing of the contents of syringe 3 and 4, the mixture is pushed into one of the syringes, which is then attached onto a second inlet of the applicator. The formulation is applied to a tissue surface as specified by the applicator manufacturer.

### Example 8

### Drug Combinations in PEG-SG4

A 1 ml syringe (syringe 1) equipped with a luer-lock mixing connector is filled with PEG-SG4 (100 mg). A 1 ml capped syringe (syringe 2) is filled with 0.25 ml of 6.3 mM HCl solution (pH 2.1). A 1 ml capped syringe (syringe 3) is filled with 0.25 ml 0.12 M monobasic sodium phosphate and 0.2 M sodium carbonate (pH 9.7) buffer. A 1 ml syringe (syringe 4) equipped with luer-lock mixing connector is filled with 5 mg a drug combination, amoxapine and prednisolone, which is previously sieved through a 100 um stainless steel sieve. The contents of syringe 4 and syringe 2 are mixed through a mixing connector by repeatedly transferring the contents from one syringe to the other. This solution is then used to reconstitute the solids in syringe 1. After complete mixing, all of the formulation is pushed into one of the syringes that is then attached to one inlet of an applicator (MICROMEDICS air assisted spray-applicator (Model SA-6105)). Syringe 3 containing the pH 9.7 solution is attached onto the other inlet of the applicator. The formulation is applied to a tissue surface as specified by the applicator manufacturer.

This process for preparing drug combination-containing in-situ forming hydrogels may be used to prepare in situ forming hydrogels that containing other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 9

### Drug Combinations in Premix

A 1 ml syringe (syringe 1) equipped with a luer-lock mixing connector is filled with a mixture of PEG-SH4 (50 mg), PEG-SG4 (50 mg), and a drug combination, amoxapine and prednisolone (5 mg, sifted to less than 100 micron using a 100 micron stainless steel sieve). A 1 ml capped syringe (syringe 2) is filled with 0.25 ml of 6.3 mM HCI solution (pH 2.1). A 1 ml capped syringe (syringe 3) is filled with 0.35 ml 0.24 M monobasic sodium phosphate and 0.4 M sodium carbonate (pH 10.0) buffer. The components are mixed and applied to a tissue surface using the procedure described in Example 7. This process for preparing drug combination-containing in-situ forming hydrogels may be used to prepare in-situ forming hydrogel containing other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 10

### Two Drugs Combined for Incorporation into an in situ Forming Hydrogel

A 1 ml syringe (syringe 1) equipped with a luer-lock mixing connector is filled with a mixture of PEG-SG4 (50 mg) and PEG-SH4 (50 mg). A 1 ml capped syringe (syringe 2) is filled with 0.25 ml of 6.3 mM HCl solution (pH 2.1). A 1 ml capped syringe (syringe 3) is filled with 0.25 ml 0.12 M monobasic sodium phosphate and 0.2 M sodium carbonate (pH 9.7) buffer. Amoxapine (1 mg) and Prednisolone (1 mg), both sifted to less than 100 micron through a 100 micron stainless steel sieve, are then added to syringe 1. The components are mixed and applied to a tissue surface using the procedure described in Example 7.

This process may be used to incorporate other drug combinations into in situ forming hydrogel, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 11

### Drug Combination Loaded Microspheres in Premix

A 1 ml syringe (syringe 1) equipped with a luer-lock mixing connector is filled with a mixture of PEG-SG4 (50 mg), PEG-SH4 (50 mg), and 2.7% of a drug combination (amoxapine and prednisolone) loaded MePEG5000-PDLLA (65:35) microspheres prepared by spray drying (0.5 or 2 mg) (prepared using the procedure described in Example 1). A 1 ml capped syringe (syringe 2) is filled with 0.25 ml of 6.3 mM HCl solution (pH 2.1). A 1 ml capped syringe (syringe 3) is filled 0.25 ml 0.12 M monobasic sodium phosphate and 0.2 M sodium carbonate (pH 9.7) buffer. The components are mixed and applied to a tissue surface using the procedure described in Example 7. The above process is repeated using the microspheres prepared in Examples 87, 88 and 91.

This process for preparing drug combination-containing in situ forming hydrogel may be used to prepare in situ forming hydrogel containing other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 12

### Incorporation of Drug Combination Loaded Micelles into Premix

A 1 ml syringe (syringe 1) equipped with a luer-lock mixing connector is filled with a mixture of PEG-SG4 (50 mg) and PEG-SH4 (50 mg). A 2 ml serum vial is filled with 1.5 ml of 6.3 mM HCl solution (pH 2.1). A 1 ml capped syringe (syringe 2) is filled with 0.25 ml 0.12 M monobasic sodium phosphate and 0.2 M sodium carbonate (pH 9.7) buffer. A 2 ml serum vial is filled with a drug combination (amoxapine and prednisolone) loaded micelles (2 mg or 8 mg) (prepared as in Example 4) and reconstituted with 1 ml of the pH 2.1 solution. 0.25 ml of the micelle solution is removed with a 1 ml syringe; the syringe is attached to syringe 1 containing the solids PEG-SG4 and PEG-SH4; and the components are mixed through the mixing connector by repeatedly transferring the contents from one syringe to the other. After complete mixing, the entire mixture is pushed into one of the syringes, which is then attached to one inlet of an applicator (MICROMEDICS air assisted spray-applicator (Model SA-6105)). Syringe 3 containing the pH 9.7 solution is attached onto the other inlet of the applicator. The formulation is applied to a tissue surface as specified by the applicator manufacturer.

This process may be used to incorporate micelles loaded with other drug combinations into premix, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 13

### Tetra Functional Poly (Ethylene Glycol) Succinimidyl Glutarate (PEG-SG4), Non Gelling Formulation

A 3 ml syringe (syringe 1) equipped with a luer-lock mixing connector is filled with containing PEG-SG4 (400mg). A 3 ml capped syringe (syringe 2) is filled with 1.0 ml of 6.3 mM HCl solution (pH 2.1). A 3 ml capped syringe (syringe 3) is filled 1 ml 0.12 M monobasic sodium phosphate and 0.2 M sodium carbonate (pH 9.7) buffer. The components are mixed and applied to a tissue surface using the procedure described in Example 7.

### Example 14

### Gelling Formulation (Premix) II

A 3 ml syringe (syringe 1) equipped with a luer-lock mixing connector is filled with a mixture of PEG-SG4 (200mg) and PEG-SH4 (200 mg). A 3 ml capped syringe (syringe 2) is filled with 1.0 ml of 6.3 mM HCl solution (pH 2.1). A 3 ml capped syringe (syringe 3) is filled 1 ml 0.12 M monobasic sodium phosphate and 0.2 M sodium carbonate (pH 9.7) buffer. The components are mixed and applied to a tissue surface using the procedure described in Example 7.

### Example 15

### Drug Combination Loaded Premix

A 3 ml syringe (syringe 1) equipped with a luer-lock mixing connector is filled with a mixture of PEG-SG4 (200mg), PEG-SH4 (200 mg), and a drug combination (amoxapine and prednisolone) (7 mg or 14 mg). A 3 ml capped syringe (syringe 2) is filled with 1 ml of 6.3 mM HCl solution (pH 2.1). A 3 ml capped syringe (syringe 3) is filled 1.5 ml 0.24 M monobasic sodium phosphate and 0.4 M sodium carbonate (pH 10) buffer. The components are mixed and applied to a tissue surface using the procedure described in Example 7.

This process may be used to form premix loaded with other drug combinations, including but not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 16

### Screening Assay for Assessing the Effect of Various Compounds on Nitric Oxide Production by Macrophages

The murine macrophage cell line RAW 264.7 is trypsinized to remove cells from flasks and plated in individual wells of a 6-well plate. Approximately 2×10⁶ cells are plated in 2 ml of media containing 5% heat-inactivated fetal bovine serum (FBS). RAW 264.7 cells are incubated at 37° C. for 1.5 hours to allow adherence to plastic. The agent is prepared in DMSO at a concentration of 10⁻² M and serially diluted 10-fold to give a range of stock concentrations (10⁻⁸ M to 10⁻² M). Media was then removed and cells are incubated in 1 ng/ml of recombinant murine IFNγ and 5 ng/ml of LPS with or without mitoxantrone in fresh media containing 5% FBS. The agent is added to cells by directly adding agent DMSO stock solutions, prepared earlier, at a 1/1000 dilution, to each well. Plates containing IFNγ, LPS plus or minus the agent are incubated at 37° C. for 24 hours (Chem. Ber. (1879) 12: 426; J. AOAC (1977) 60-594; Ann. Rev. Biochem. (1994) 63: 175).

At the end of the 24 hour period, supernatants are collected from the cells and assayed for the production of nitrites. Each sample is tested in triplicate by aliquoting 50 μL of supernatant in a 96-well plate and adding 50 μL of Greiss Reagent A (0.5 g sulfanilamide, 1.5 ml H₃PO₄, 48.5 ml ddH₂O) and 50 μL of Greiss Reagent B (0.05 g N-(1-naphthyl)-ethylenediamine, 1.5 ml H₃PO₄, 48.5 ml ddH₂O). Optical density is read immediately on microplate spectrophotometer at 562 nm absorbance. Absorbance over triplicate wells is averaged after subtracting background and concentration values obtained from the nitrite standard curve (1 μM to 2 mM). Inhibitory concentration of 50% (IC₅₀) is determined by comparing average nitrite concentration to the positive control (cell stimulated with IFNγ and LPS). An average of n=4 replicate experiments is used to determine IC₅₀ values for the agent.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

### Example 17

### Screening Assay for Assessing the Effect of Various Anti-Scarring Drug Combinations on TNF-alpha Production by Macrophages

The human macrophage cell line, THP-1 is plated in a 12 well plate such that each well contains 1 X 106 cells in 2 ml of media containing 10% FCS. Opsonized zymosan is prepared by resuspending 20 mg of zymosan A in 2 ml of ddH₂O and homogenizing until a uniform suspension is obtained. Homogenized zymosan is pelleted at 250 g and resuspended in 4 ml of human serum for a final concentration of 5 mg/ml and incubate in a 37° C. water bath for 20 minutes to enable opsonization. An agent is prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol Meth. (2000) 235 (1-2): 33-40).

THP-1 cells are stimulated to produce TNFα by the addition of 1 mg/ml opsonized zymosan. The agent is added to THP-1 cells by directly adding DMSO stock solutions, prepared earlier, at a 1/1000 dilution, to each well. Each drug concentration is tested in triplicate wells. Plates are incubated at 37° C. for 24 hours.

After 24 hour stimulation, supernatants are collected to quantify TNFα production. TNFα concentrations in the supernatants are determined by ELISA using recombinant human TNFα to obtain a standard curve. A 96-well MaxiSorb plate is coated with 100 μL of anti-human TNFα Capture Antibody diluted in Coating Buffer (0.1M sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/8 and 1/16; (b) recombinant human TNFα is prepared at 500 μg/ml and serially diluted to yield as standard curve of 7.8 pg/ml to 500 μg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human TNFα detection antibody+avidin-HRP) for 1 hour at room temperature. Following this incubation, the plates are washed 7 times and 100 μL of Substrate Solution (tetramethylbenzidine, H₂O₂) is added to plates and incubated for 30 minutes at room temperature. Stop Solution (2 N H₂SO₄) is then added to the wells and a yellow color reaction is read at 450 nm with λ correction at 570 nm. Mean absorbance is determined from triplicate data readings and the mean background is subtracted. TNFα concentration values are obtained from the standard curve. Inhibitory concentration of 50% (IC₅₀) is determined by comparing average TNFα concentration to the positive control (THP-1 cells stimulated with opsonized zymosan). An average of n=4 replicate experiments are used to determine IC₅₀ values.

Exemplary drug combinations or their individual components that may be tested in this model include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 18

### Surgical Adhesions Model to Assess Fibrosis Inhibiting drug Combinations or Individual Components Thereof in Rats

The rat caecal sidewall model is used to as to assess the anti-fibrotic capacity of formulations in vivo. Sprague Dawley rats are anesthetized with halothane. Using aseptic precautions, the abdomen is opened via a midline incision. The caecum is exposed and lifted out of the abdominal cavity. Dorsal and ventral aspects of the caecum are successively scraped a total of 45 times over the terminal 1.5 cm using a #10 scalpel blade. Blade angle and pressure are controlled to produce punctate bleeding while avoiding severe tissue damage. The left side of the abdomen is retracted and everted to expose a section of the peritoneal wall that lies proximal to the caecum. The superficial layer of muscle (transverses abdominis) is excised over an area of 1×2 cm², leaving behind torn fibers from the second layer of muscle (internal oblique muscle). Abraded surfaces are tamponaded until bleeding stops. The abraded caecum is then positioned over the sidewall wound and attached by two sutures. The formulation is applied over both sides of the abraded caecum and over the abraded peritoneal sidewall. A further two sutures are placed to attach the caecum to the injured sidewall by a total of 4 sutures and the abdominal incision is closed in two layers. After 7 days, animals are evaluated post mortem with the extent and severity of adhesions being scored both quantitatively and qualitatively.

Exemplary drug combinations or their individual components that may be tested in this model include, but are not limited to, amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 19

### Surgical Adhesions Model to Assess Fibrosis Inhibiting Drug Combinations or Individual Components Thereof in Rabbits

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 20

### Screening Assay for Assessing the Effect of Various Compounds on Cell Proliferation

Fibroblasts at 70-90% confluency are trypsinized, replated at 600 cells/well in media in 96-well plates and allowed to attach overnight. A drug combination or individual component(s) thereof is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and the agent are incubated at 37° C. for 72 hours (In vitro toxicol. (1990) 3: 219; Biotech. Histochem. (1993) 68: 29; Anal. Biochem. (1993) 213: 426).

To terminate the assay, the media is removed by gentle aspiration. A 1/400 dilution of CYQUANT 400X GR dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Inhibitory concentration of 50% (IC₅₀) is determined by taking the average of triplicate wells and comparing average relative fluorescence units to the DMSO control. An average of n=4 replicate experiments is used to determine IC₅₀ values.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 21

### Evaluation of Drug Combinations Formulations on Intimal Hyperplasia Development in a Rat Balloon Injury Carotid Artery Model as an Example to Evaluate fibrosis Inhibiting Agents

A rat balloon injury carotid artery model is used to demonstrate the efficacy of a paclitaxel containing mesh system on the development of intimal hyperplasia fourteen days following placement.

Control Group

Wistar rats weighing 400-500 g are anesthetized with 1.5% halothane in oxygen and the left external carotid artery is exposed. An A 2 French FOGARTY balloon embolectomy catheter (Baxter, Irvine, Calif.) is advanced through an arteriotomy in the external carotid artery down the left common carotid artery to the aorta. The balloon is inflated with enough saline to generate slight resistance (approximately 0.02 ml) and it is withdrawn with a twisting motion to the carotid bifurcation. The balloon is then deflated and the procedure repeated twice more. This technique produced distension of the arterial wall and denudation of the endothelium. The external carotid artery is ligated after removal of the catheter. The right common carotid artery is not injured and is used as a control.

Local Perivascular Drug Combination Treatment

Immediately after injury of the left common carotid artery, a 1 cm long distal segment of the artery is exposed and treated with the in situ forming hydrogels as described in examples 9,10,11 and 12. The wound is then closed and the animals are kept for 14 days.

Histology and Immunohistochemistry

At the time of sacrifice, the animals are euthanized with carbon dioxide and pressure perfused at 100 mmHg with 10% phosphate buffered formaldehyde for 15 minutes. Both carotid arteries are harvested and left overnight in fixative. The fixed arteries are processed and embedded in paraffin wax. Serial cross-sections are cut at 3 μm thickness every 2 mm within and outside the implant region of the injured left carotid artery and at corresponding levels in the control right carotid artery. Cross-sections are stained with Mayer's hematoxylin-and-eosin for cell count and with Movat's pentachrome stains for morphometry analysis and for extracellular matrix composition assessment.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 22

### Effect of Paclitaxel and Other Anti-Microtubule Agents on Matrix Metalloproteinase Production

Materials and Methods

IL-1 stimulated AP-1 Transcriptional Activity is Inhibited by Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations. Paclitaxel treatment decreased CAT activity in a concentration dependent manner (mean±SD). The data noted with an asterisk (*) have significance compared with IL-1-induced CAT activity according to a t-test, P<0.05. The results shown are representative of three independent experiments.

Effect of Paclitaxel on IL-1 Induced AP-1 DNA Binding Activity, AP-1 DNA

Binding activity was assayed with a radiolabeled human AP-1 sequence probe and gel mobility shift assay. Extracts from chondrocytes untreated or treated with various amounts of paclitaxel (10⁻⁷to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

Effect of Paclitaxel on IL-1 Induced MMP-1 and MMP-3 mRNA Expression

Cells were treated with paclitaxel at various concentrations (10⁻⁷ to 10⁻⁵ M) for 24 hours, then treated with IL-1β (20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels were conducted. The MMP-1 and MMP-3 expression levels were normalized with GAPDH.

Effect of Other Anti-microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1% denatured gel, transferred to a nylon membrane and hydridized with the ³²P-labeled collagenase cDNA probe. ³²P-labeled glyceraldehyde phosphate dehydrase (GAPDH) cDNA as an internal standard to ensure roughly equal loading. The exposed films were scanned and quantitatively analyzed with IMAGEQUANT.

Results

Promoters on the Family of Matrix Metalloproteinases

FIG. 1 shows that all matrix metalloproteinases contained the transcriptional elements AP-1 and PEA-3 with the exception of gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

Effect of Paclitaxel on AP-1 Transcriptional Activity

As demonstrated in FIG. 2, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

Effect of Paclitaxel on AP-1 DNA Binding Activity

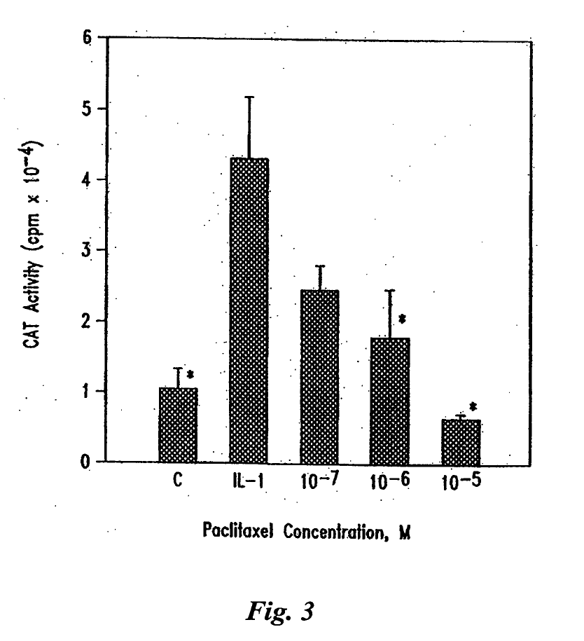

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 3, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 4, IL-1 induction increases collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

Effect of Other Anti-microtubules on Collagenase Expression

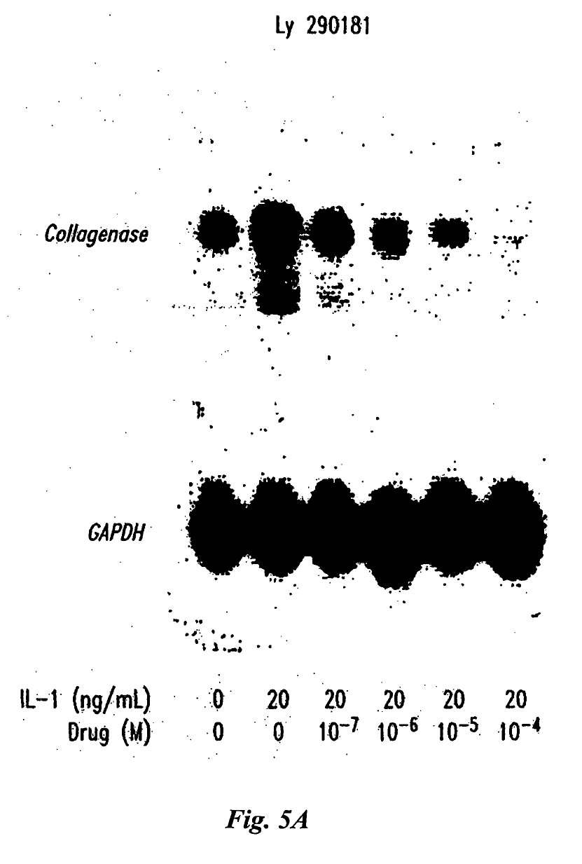

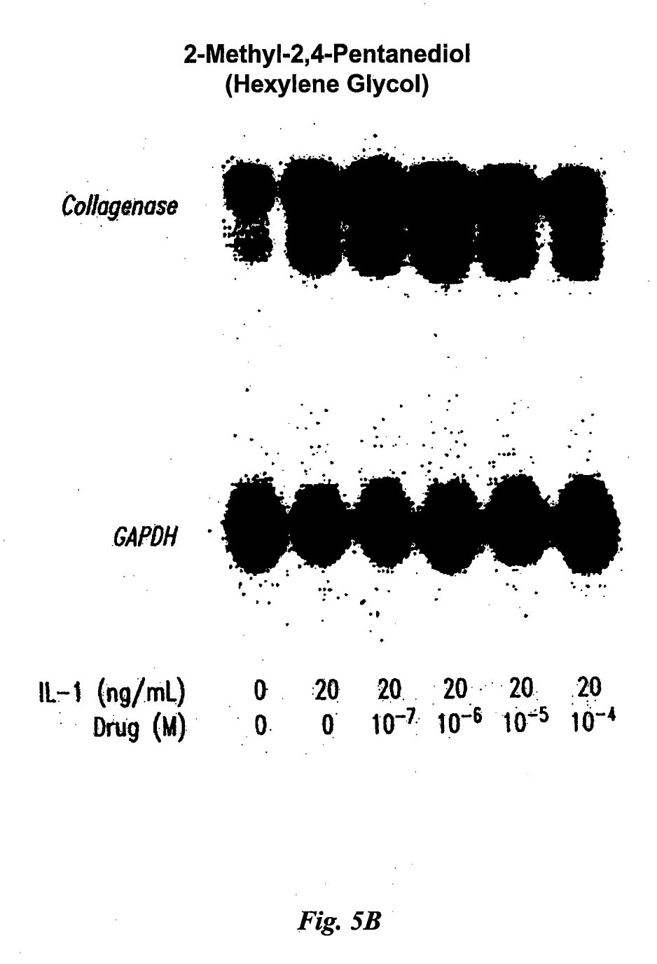

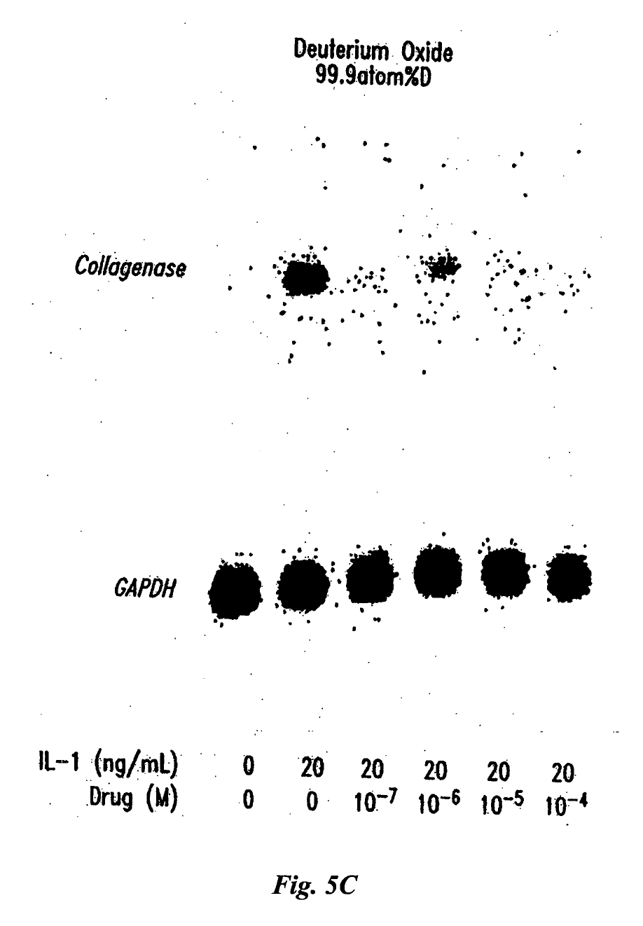

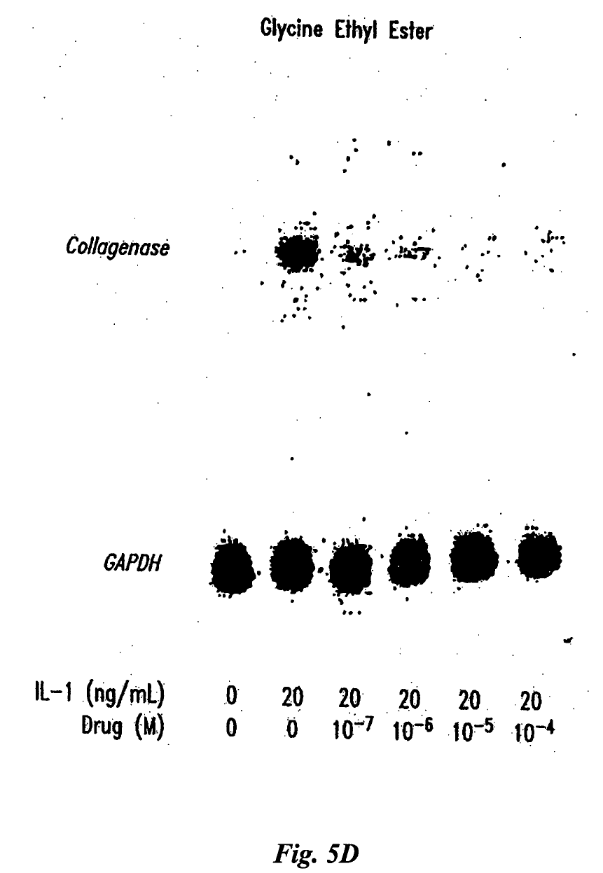

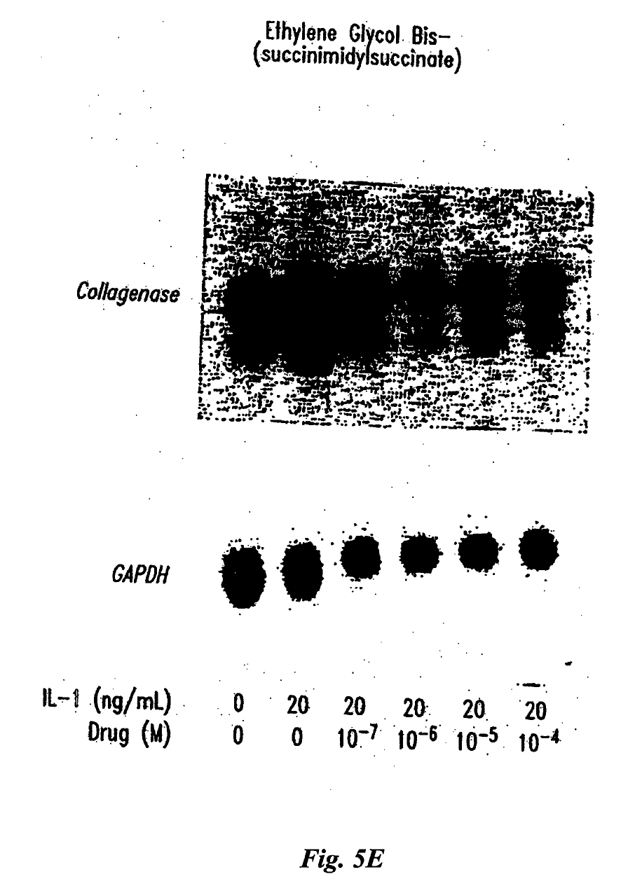

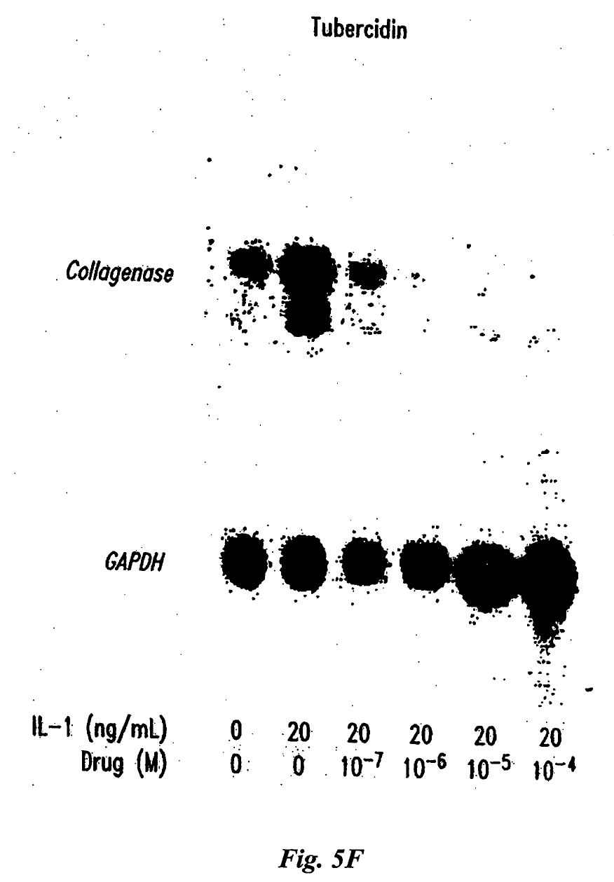

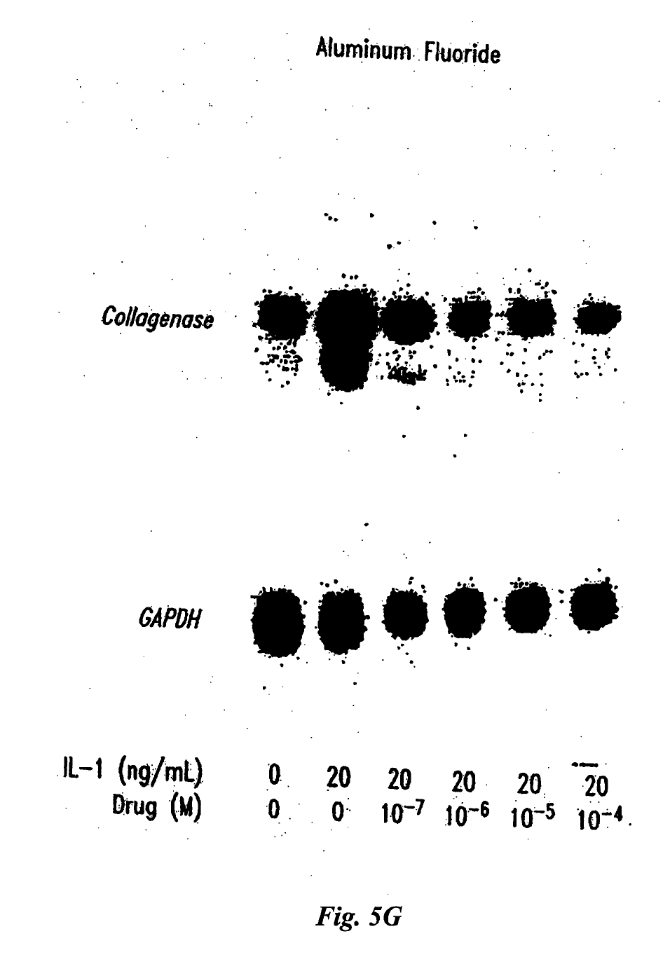

FIGS. 5A-H demonstrate that anti-microtubule agents inhibited collagenase expression. Expression of collagenase was stimulated by the addition of IL-1 which is a proinflammatory cytokine. Pre-incubation of chondrocytes with various anti-microtubule agents, specifically LY290181, hexylene glycol, deuterium oxide, glycine ethyl ester, ethylene glycol bis-(succinimidylsuccinate), tubercidin, AIF₃, and epothilone, all prevented IL-1-induced collagenase expression at concentrations as low as 1×10⁻⁷ M.

Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases which are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin can have a beneficial effect in inflammatory diseases.

In addition to paclitaxel's inhibitory effect on collagenase expression, LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 23

### Inhibition of Angiogenesis by Paclitaxel

Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 ul aliquot of 0.5% aqueous methylcellulose. Since paclitaxel is insoluble in water, glass beads were used to produce fine particles. Ten microliter aliquots of this solution were dried on parafilm for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1 M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (see the table below). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

Avascular Gradient

| | |
|---|---|
| 0 | normal vascularity |
| 1 | lacking some microvascular movement |
| 2* | small avascular zone approximately 2 mm in diameter |
| 3* | avascularity extending beyond the disk (6 mm in diameter) |

*indicates a positive antiangiogenesis response

The dose-dependent, experimental data of the effects of paclitaxel at different concentrations are shown in the Table 11 below.

TABLE 11

| Agent | Delivery Vehicle | Concentration | Inhibition/n |
|---|---|---|---|
| paclitaxel | methylcellulose (10 ul) | 0.25 ug | 2/11 |
| | methylcellulose (10 ul) | 0.5 ug | 6/11 |
| | methylcellulose (10 ul) | 1 ug | 6/15 |
| | methylcellulose (10 ul) | 5 ug | 20/27 |
| | methylcellulose (10 ul) | 10 ug | 16/21 |
| | methylcellulose (10 ul) | 30 ug | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 24

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media and diluted to a concentration of 2-2.5×10⁵ cells/ml in migration media. Migration media consists of phenol red free Dulbecco's Modified Eagle Medium (DMEM) containing 0.35% human serum albumin. A 100 μL volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/ml in a total volume of 150 μL. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

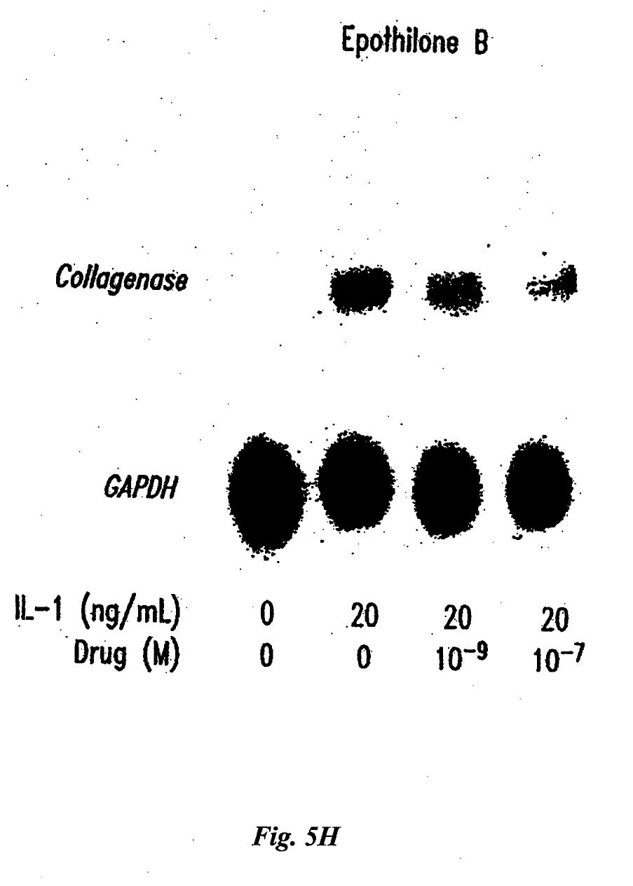

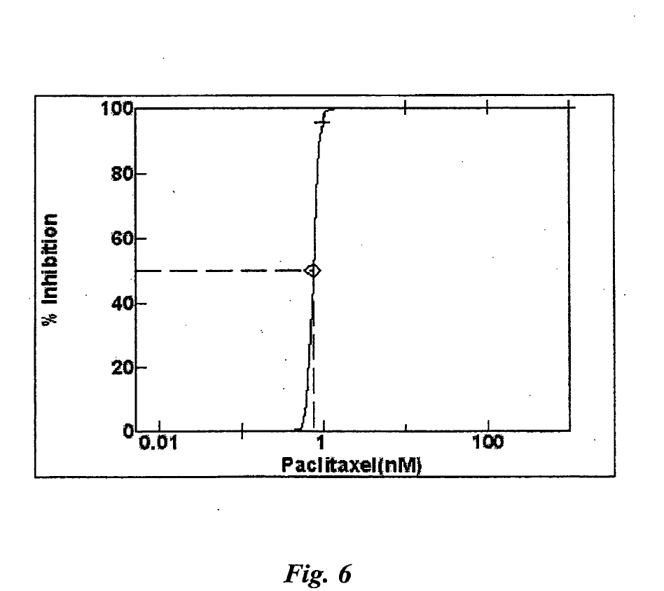

At the end of the 4 hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). See FIG. 6 (IC₅₀=0.76 nM). References: Biotechniques (2000) 29: 81; J. Immunol Methods (2001) 254: 85.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 25

### Screening Assay for Assessing the Effect of Various Compounds on IL-1β Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization.

Geldanamycin was prepared in DMSO at a concentration of 10⁻² M and serially diluted 10-fold to give a range of stock concentrations (10⁻⁸ M to 10⁻² M).

THP-1 cells were stimulated to produce IL-1β by the addition of 1 mg/ml opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

After a 24 hour stimulation, supernatants were collected to quantify IL-1β production. IL-1β concentrations in the supernatants were determined by ELISA using recombinant human IL-1β to obtain a standard curve. A 96-well MaxiSorb plate was coated with 100 μL of anti-human IL-1β Capture Antibody diluted in Coating Buffer (0.1 M Sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/4 and 1/8; (b) recombinant human IL-1β was prepared at 1000 μg/ml and serially diluted to yield as standard curve of 15.6 μg/ml to 1000 μg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human IL-1β detection antibody+avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with λ correction at 570 nm. Mean absorbance was determined from triplicate data readings and the mean background was subtracted. IL-1β concentration values were obtained from the standard curve. Inhibitory concentration of 50% (IC50) was determined by comparing average IL-1β concentration to the positive control (THP-1 cells stimulated with opsonized zymosan).

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

References: J. Immunol. (2000) 165: 411-418; J. Immunol. (2000) 164: 4804-4811; J. Immunol Meth. (2000) 235 (1-2): 33-40.

### Example 26

### Screening Assay for Assessing the effect of Various Compounds on IL-8 Production by Macrophages

The human macrophage cell line, THP-1 is plated in a 12 well plate such that each well contains 1×10⁶ cells in 2 ml of media containing 10% FCS. Opsonized zymosan is prepared by resuspending 20 mg of zymosan A in 2 ml of ddH₂O and homogenizing until a uniform suspension is obtained. Homogenized zymosan is pelleted at 250 g, resuspended in 4 ml of human serum for a final concentration of 5 mg/ml, and incubated in a 37° C. water bath for 20 minutes to enable opsonization. The agent is prepared in DMSO at a concentration of 10⁻² M and serially diluted 10-fold to give a range of stock concentrations (10⁻⁸ M to 10⁻² M).

THP-1 cells are stimulated to produce IL-8 by the addition of 1 mg/ml opsonized zymosan. Geldanamycin is added to THP-1 cells by directly adding DMSO stock solutions, prepared earlier, at a 1/1000 dilution, to each well. Each drug concentration is tested in triplicate wells. Plates are incubated at 37° C. for 24 hours.

After a 24 hour stimulation, supernatants are collected to quantify IL-8 production. IL-8 concentrations in the supernatants are determined by ELISA using recombinant human IL-8 to obtain a standard curve. A 96-well MAXISORB plate is coated with 100 μL of anti-human IL-8 Capture Antibody diluted in Coating Buffer (0.1M sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/100and 1/1000; (b) recombinant human IL-8 is prepared at 200 μg/ml and serially diluted to yield as standard curve of 3.1 pg/ml to 200 μg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human IL-8 detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 N H₂SO₄) is then added to the wells and a yellow color reaction is read at 450 nm with λ correction at 570 nm. Mean absorbance is determined from triplicate data readings and the mean background is subtracted. IL-8 concentration values are obtained from the standard curve. Inhibitory concentration of 50% (IC₅₀) is determined by comparing average IL-8 concentration to the positive control (THP-1 cells stimulated with opsonized zymosan). Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

References: J. Immunol. (2000) 165: 411-418; J. Immunol. (2000) 164: 4804-4811; J. Immunol Meth. (2000) 235 (1-2): 33-40.

### Example 27

### Screening Assay for Assessing the effect of Various Compounds on MCP-1 Production by Macrophages

The human macrophage cell line, THP-1 is plated in a 12 well plate such that each well contains 1×10⁶ cells in 2 ml of media containing 10% FCS. Opsonized zymosan is prepared by resuspending 20 mg of zymosan A in 2 ml of ddH₂O and homogenizing until a uniform suspension is obtained. Homogenized zymosan is pelleted at 250 g and resuspended in 4 ml of human serum for a final concentration of 5 mg/ml and incubated in a 37° C. water bath for 20 minutes to enable opsonization. The agent is prepared in DMSO at a concentration of 10⁻² M and serially diluted 10-fold to give a range of stock concentrations (10⁻⁸ M to 10⁻² M).

THP-1 cells are stimulated to produce MCP-1 by the addition of 1 mg/ml opsonized zymosan. Eldanamycin is added to THP-1 cells by directly adding DMSO stock solutions, prepared earlier, at a 1/1000 dilution, to each well. Each drug concentration is tested in triplicate wells. Plates are incubated at 37° C. for 24 hours.

After 24 hour stimulation, supernatants are collected to quantify MCP-1 production. MCP-1 concentrations in the supernatants are determined by ELISA using recombinant human MCP-1 to obtain a standard curve. A 96-well MaxiSorb plate is coated with 100 μL of anti-human MCP-1 Capture Antibody diluted in Coating Buffer (0.1M sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/100 and 1/1000; (b) recombinant human MCP-1 is prepared at 500 μg/ml and serially diluted to yield as standard curve of 7.8 μg/ml to 500 μg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human MCP-1 detection antibody+avidin-HRP) for 1 hour at room temperature. Following this incubation, the plates are washed 7 times and 100 μL of Substrate Solution (tetramethylbenzidine, H₂O₂) is added to plates and incubated for 30 minutes at room temperature. Stop Solution (2 N H₂SO₄) is then added to the wells and a yellow color reaction was read at 450 nm with λ correction at 570 um. Mean absorbance is determined from triplicate data readings and the mean background is subtracted. MCP-1 concentration values were obtained from the standard curve. Inhibitory concentration of 50% (IC₅₀) is determined by comparing average MCP-1 concentration to the positive control (THP-1 cells stimulated with opsonized zymosan).

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

References: J. Immunol. (2000) 165: 411-418; J. Immunol. (2000) 164: 4804-4811; J. Immunol Meth. (2000) 235 (1-2): 33-40.

### Example 28

### Screening Assay for Assessing the effect of Paclitaxel on Cell Proliferation

Smooth muscle cells at 70-90% confluency were trypsinized, replated at 600 cells/well in media in 96-well plates and allowed to attachment overnight. Paclitaxel was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μL/well. Each drug concentration was tested in triplicate wells. Plates containing cells and paclitaxel were incubated at 37° C. for 72 hours.

To terminate the assay, the media was removed by gentle aspiration. A 1/400 dilution of CYQUANT 400X GR dye indicator (Molecular Probes; Eugene, Oreg.) was added to 1×Cell Lysis buffer, and 200 μL of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=3 replicate experiments was used to determine IC₅₀ values.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

This assay also may be used assess the effect of compounds on proliferation of fibroblasts and murine macrophage cell line RAW 264.7.

Reference: In vitro toxicol. (1990) 3: 219; Biotech. Histochem. (1993) 68: 29; Anal. Biochem. (1993) 213: 426.

### Example 29

### Perivascular Administration of Drug Combination to Assess Inhibition of Fibrosis

WISTAR rats weighing 250-300 g are anesthetized by the intramuscular injection of Innovar (0.33 ml/kg). Once sedated, they are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 French Fogarty balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

Immediately after injury of the left common carotid artery, a 1 cm long distal segment of the artery is exposed and treated with a 0.8×0.8 cm drug-loaded material (as prepared in Examples 85 and 86) is wrapped circumferentially around the exposed artery. Two Prolene 7-0 sutures are used to hold the ends of the materials together. The wound is then closed and the animals are kept for 14 days.

Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above. combinations.

### Example 30

### MIC Determination by Microtitre Broth Dilution Method

A. MIC Assay of Various Gram Negative and Positive Bacteria

MIC assays were conducted essentially as described by Amsterdam, D. 1996, “Susceptibility testing of antimicrobials in liquid media”, p. 52-111, in Loman, V., ed. Antibiotics in laboratory medicine, 4th ed. Williams and Wilkins, Baltimore, Md. Briefly, a variety of compounds were tested for antibacterial activity against isolates of P. aeruginosa, K pneumoniae, E. coli, S. epidermidus and S. aureus in the MIC (minimum inhibitory concentration assay under aerobic conditions using 96 well polystyrene microtitre plates (Falcon 1177), and Mueller Hinton broth at 37° C incubated for 24h. (MHB was used for most testing except C721 (S. pyogenes), which used Todd Hewitt broth, and Haemophilus influenzae, which used Haemophilus test medium (HTM)) Tests were conducted in triplicate. The results are provided below in the Table 12 below.

TABLE 12
MINIMUM INHIBITORY CONCENTRATIONS OF THERAPEUTIC AGENTS
AGAINST VARIOUS GRAM NEGATIVE AND POSITIVE BACTERIA

| | Bactrial Strain | | | | | |
|---|---|---|---|---|---|---|
| | P. aeruginosa | K. pneumoniae | E. coli | S. aureus | | |
| | PAE/K799 | ATCC13883 | UB1005 | ATCC25923 | S. epidermidis | S. pyogenes |
| | H187 | C238 | C498 | C622 | C621 | C721 |
| | Wt | wt | wt | wt | wt | wt |
| Drug | Gram− | Gram− | Gram− | Gram+ | Gram+ | Gram+ |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5-fluorouracil | 10⁻⁵ | 10⁻⁶ | 10⁻⁶ | 10⁻⁷ | 10⁻⁷ | 10⁻⁴ |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2- | N | N | N | N | N | N |
| mercaptopurine | | | | | | |
| 6- | N | N | N | N | N | N |
| mercaptopurine | | | | | | |
| Cytarabine | N | N | N | N | N | N |

Activities are in Molar concentrations
Wt = wild type
N = No activity

MIC of Antibiotic-resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea and cytarabine were tested for antibacterial activity against clinical isolates of a methicillin resistant S. aureus and a vancomycin resistant pediocoocus clinical isolate in an MIC assay as described above. Compounds which showed inhibition of growth (MIC value of <1.0×10-3) included: mitoxantrone (both strains), methotrexate (vancomycin resistant pediococcus), 5-fluorouracil (both strains), etoposide (both strains), and 2-mercaptopurine (vancomycin resistant pediococcus).

### Example 31

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1L HPLC grade water is added and the solution is stirred until all the salts are dissolved. If required, the pH of the solution is adjusted to pH 7.4±0.2 using either 0.1N NaOH or 0.1N phosphoric acid.

### Example 32

### Release study to Determine Release Profile of a Therapeutic Agent From a Polymeric Composition

The release profile of a therapeutic agent from a polymeric composition can be determined according to the following procedure.

Release and Extraction

A sample is placed in a 16×125 mm screw capped culture tube. 16 ml release buffer (Example 31) is added to the tube. The samples are placed on a rotating wheel (30 rpm) in a 37° C. oven. At the various time intervals (2 h, 5 h, 8 h, 24 h and then daily), the sample tubes are taken from the oven, placed in a rack and the caps are removed in a fume hood. As much of the release buffer as possible is removed from the tube and placed in a second culture tube. 16 ml of release media is then added to the sample containing tube using an Oxford pipettor bottle. The samples are capped with a new PTFE lined cap. All samples are returned to the rotating wheel device in the oven.

Using a p1000 pipettor (PIPETMAN) and a clean pipette tip, remove and discard 1 ml of release media from each sample. Add 1 ml of dichloromethane to each sample using an oxford pipettor bottle. Cap each sample tube with the respective PTFE lined screw cap. Hand shake each sample vigorously for 5 seconds. Place samples on the labquake rotator and rotate for 15 min. Centrifuge samples at 1500 rpm for 10 minutes.

Transfer the sample tubes to a fume hood and uncap. Remove most of the supernatant (aqueous phase) using a Pasteur pipette and vacuum system. Remove the final portion of the supernatant with a glass syringe. Transfer sample tubes to the pierce drying system, set the heating block to 1.5 (45° C.) and turn on the system. Dry all samples on the pierce drying system under a stream of nitrogen gas (approximately 45 min.). Re-cap the sample tubes, place in a plastic bag, label bag with date and time of sample, and store at −20° C. (freezer) until analysis.

External Standard Preparation

100 mg of the drug to be analysed is accurately weighed, quantitatively transferred and made up to volume with ACN in a 100 ml volumetric flask (1 mg/ml). 5 ml of this standard solution is transferred, using a volumetric pipette, to a 100 ml volumetric flask and is made up to volume with ACN (50 μg/ml). Serial dilutions (5 ml qs ad 10 ml with ACN) are used to prepare 25, 12.5, 6.25, 3.13, 1.56, 0.781 and 0.391 μg/ml solutions respectively. On the day of HPLC analysis of samples, an aliquot (−100 μl) of each standard is placed into separate autosampler vials using small volume inserts and is transferred to the HPLC.

Sample Reconstitution

Remove samples to be analyzed from the freezer, place in a fume hood, and allow tubes to come to room temperature. Uncap and add 1 ml of water/acetonitrile (50/50) to each tube with an Oxford pipettor. Recap sample tubes and vortex for 60 s.

Centrifuge sample tubes at 1500 rpm for 15 min. In a fume hood, transfer approximately 500 μl of each sample to a separate HPLC autosampler vial with a clean Pasteur pipette. Cap each autosampler vial and transfer to the HPLC. Dispose of the sample tube and Pasteur pipette. The samples are then analysed for drug content using HPLC.

### Example 33

### Formulation of a drug Combination in a Vehicle Comprising a triblock Copolymer

A drug combination (amoxapine and prednisolone) is incorporated into a formulation comprising a triblock copolymer and a diluent (described below) by dissolving the drug combination in the diluent with stirring at ambient temperature for at least two hours, then adding the triblock copolymer, again with stirring for at least 2 hours. Longer periods of time are used to add triblock copolymer at higher concentrations. For example, the addition of 33% triblock copolymer is accomplished by stirring for at least 15 hours (overnight). The diluent is PEG 300 NF or PEG 400 derivatized by end addition of trimethylene carbonate 90%/glycolide 10% in a ratio of 400:100. The triblock copolymer is an ABA copolymer with blocks A containing polymerized trimethylene carbonate (90%) and glycolide (10%), having a total molecular weight of about 900 g/mol and the B block containing PEG 400. The drug combination (amoxapine and prednisolone) is effectively incorporated into this formulation at a concentration of 0.015 to 0.45 mg/ml. The amount of triblock copolymer in the formulation is varied from 2.3 to 50%w/w using PEG 400 as the diluent. The product is sterilized by exposure to about 2.5 kGy of gamma radiation.

This process may be used to prepare formulations of other drug combinations in a vehicle comprising a triblock copolymer. Such other drug combinations include, but are not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 34

### Fromulation of a Drug Combination in a Co-Solvent Vehicle

A drug combination (amoxapine and prednisolone) is incorporated into a formulation comprising water and PEG 300 NF. The drug combination (amoxapine and prednisolone) is first dissolved in a 90:10 mixture of PEG 300 NF:water by stirring at ambient temperature for at least two hours. Once the drug was dissolved, the composition is combined with equal parts of a 50:50 mixture of PEG 300 NF:water. The final composition is the drug combination dissolved in a mixture of 70:30 PEG 300 NF:water. The drug combination (amoxapine and prednisolone) is incorporated at concentrations of 0.45 to 4.5 mg/ml. The composition is passed through a 0.22 μm filter to render it sterile.

This process may be used to prepare formulations of other drug combinations in a co-solvent vehicle. Such other drug combinations include, but are not limited to, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 35

### Determination the Maximum Tolerated Dose (MTD) of a Drug After Intra-Articular Injection

Male Hartley guinea pigs, at least 6 weeks old, were anaesthetized using 5% isoflurane in an enclosed chamber. The animals were weighed and then transferred to the surgical table where anesthesia was maintained by nose cone with 2% isoflurane. The knee area on both legs was shaved and knee width at the head of the femur was measured on both knees. The skin on the right knee was sterilized. A 25G needle was introduced into the synovial cavity using a medial approach and 0.1 mL of the test formulation was injected. Three or seven days after the injection, the animals were sacrificed by cardiac injection of 0.7 mL Euthanyl under deep anesthesia (5% isoflurane). Sample size was N=3 for each formulation.

Knee function was assessed before sacrifice by recording changes in walking behavior and signs of tenderness. The animal was weighed immediately after sacrifice. The width of both knees at the head of the femur was then measured with calipers. The knee joint was dissected open by transecting the quadriceps tendon, cutting through the lateral and medial articular capsule and flipping the patella over the tibia. Knee inflammation was assessed by recording signs of swelling, vascularization, fluid accumulation and change in color in subcutaneous tissue as well as inner joint structures. Photographs were taken to document findings. All data was recorded by observers blinded to the treatment groups.

The MTD of the drug in the test formulation was determined to be that for which knee inflammation was not observed.

The MTD of paclitaxel in the triblock gel formulation according to Example 34 was found to be 0.075 mg/ml, based upon evaluation at 7 days. Evaluation of this formulation after three days showed that doses up to 0.15 mg/ml were tolerated. The 0.015 mg/ml dose showed signs of inflammation only after seven days. The MTD of paclitaxel in the co-Solvent formulation was found to be 1.5 mg/ml, based upon a 3 day evaluation.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 36

### Evaluation of Local Tissue Distribution of a Drug Combination or Individual Component(s) Thereof After Intra-Articular Injection

Animals are injected in the knee joint as described above in 4.2 with the drug MTD dose identified for each formulation. Three or seven days after injection the animals are euthanized with an intracardiac injection of Euthanyl. The knee joint is dissected open and the synovial membrane, the anterior cruciate ligament, the fat pad, the menisci and the cartilage are harvested. Each tissue is briefly rinsed in saline solution, blotted dry and stored individually in a scintillation vial at −20° C. until drug analysis.

The drug is extracted from a weighed pooled sample from three animals by homogenization using a Polytron PT2000 homogenizer. The instrument setting is 3 to 9 and the extraction time is 1 minute. The extraction solution is 1 mL of 50/50 acetonitrile (ACN)/water containing 0.2 μg/mL 10-deacetyl taxol (10-DAT) and 0.1% formic acid. The extract is centrifuged using a Beckman J6-HC centrifuge for 10 minutes at 3000 rpm. The supernatant is filtered through an Acrodisc CR (13 mm, 0.45μ) syringe filter into an HPLC vial for LC/MS/MS analysis. Some fat pad samples that did not produce a clear supernatant are centrifuged again prior to filtration using an IEC Micromax centrifuge for 10 minutes at 10000 rpm.

The drug content in the extract is determined by an LC/MS/MS method using an internal calibration. For example, a calibration curve may range from 0.01 to 1 μg/mL for drug with 0.2 μg/mL 10-DAT. The LC/MS/MS system consists of a Waters 2695 separation module and a Waters Micromass QuattoMicro triple-Quad mass spectrometer.

This process may be used to evaluate local tissue distribution of drug combinations or individual components thereof after intra-articular injection, including but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 37

### Evaluation of Local Tissue Distribution of a Drug Combination or Individual Component(s) Thereof After Intra-Articular Injection

Animals are treated in the manner described in Example 35. Rabbits are evaluated by intra-articular injection of 0.5 ml of formulation. Drug is extracted from individual tissue sample from three animals by homogenization using a Freezer/Mill, SPEX CertiPrep 6850. The ground sample is extracted with 12 mL solution containing acetic acid (3.4 mM) and LiCl (4 to 8 μM) in 50/50 ACN/water. Extraction is performed on a tube rotator (Labquake Shaker) for 30 minutes at room temperature. The extract is filtered through an Acrodisc CR (13 mm, 0.45μ) syringe filter into an HPLC vial for LC/MS/MS analysis.

The drug content in the extract is determined by an LC/MS/MS method using an external calibration. The calibration curve ranges from 0.01 to 1 μg/mL for paclitaxel. The LC/MS/MS system consists of a Waters 2695 separation module and a Waters Micromass QUATTOMICRO triple-Quad mass spectrometer.

This process may be used to evaluate local tissue distribution of a drug combination or individual components thereof after intra-articular injection, including but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 38

### Spinal Surgical Adhesions Model to Assess Fibrosis Inhibiting Drug Combinations or Individual Component(s) Thereof in Rabbits

Extensive scar formation and adhesions often occur after lumbar spine surgery involving the vertebrae. The dense and thick fibrous tissue adherent to the spine and adjacent muscles must be removed by surgery. Unfortunately, fibrous adhesions usually reform after the secondary surgery. Adhesions are formed by proliferation and migration of fibroblasts from the surrounding tissue at the site of surgery. These cells are responsible for the healing response after tissue injury. Once they have migrated to the wound they lay down proteins such as collagen to repair the injured tissue. Overproliferation and secretion by these cells induce local obstruction, compression and contraction of the surrounding tissues with accompanying side effects.

The rabbit laminectomy spinal adhesion model described herein is used to investigate spinal adhesion prevention by local slow release of antifibrotic drugs.

Five to six animals are included in each experimental group to allow for meaningful statistical analysis. Formulations with various concentrations of antifibrotic drugs are tested against control animals to assess inhibition of adhesion formation.

Rabbits are anesthetized with an IM injection of ketamine/zylazine. An endotracheal tube is inserted for maintenance of anesthesia with halothane. The animal is placed prone on the operating table on top of a heating pad and the skin over the lower half of the back is shaved and prepared for sterile surgery. A longitudinal midline skin incision is made from L-1 to L-5 and down the lumbosacral fascia. The fascia is incised to expose the tips of the spinous processes. The paraspinous muscles are dissected and retracted from the spinous process and lamina of L-4. A laminectomy is performed at L-4 by removal of the spinal process with careful bilateral excision of the laminae, thus creating a small 5×10 mm laminectomy defect. Hemostasis is obtained with Gelfoam. The test formulations are applied to the injury site and the wound is closed in layers with Vicryl sutures. The animals are placed in an incubator until recovery from anesthesia and then returned to their cage.

Two weeks after surgery, the animals are anesthetized using procedures similar to those described above. The animals are euthanized with Euthanyl. After a skin incision, the laminectomy site is analyzed by dissection and the amount of adhesion is scored using scoring systems published in the scientific literature for this type of injury.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 39

### Tendon Surgical Adhesions Model to Assess Fibrosis Inhibiting Agents in Rabbits

This model is used to investigate whether adhesion of the tendons can be prevented by local slow release of drugs known to inhibit fibrosis. Polymeric formulations are loaded with drugs and implanted around injured tendons in rabbits. In animals without fibrosis -inhibiting formulations, adhesions develop within 3 weeks of flexor tendon injury if immobilization of the tendon is maintained during that period. An advantage of rabbits is that their tendon anatomy and cellular behaviour during tendon healing are similar to those in man except for the rate of healing that is much faster in rabbits.

Rabbits are anesthetized and the skin over the right hindlimb is shaved and prepared for sterile surgery. Sterile surgery is performed aided by an operating microscope. A longitudinal midline skin incision is made on the volvar aspect of the proximal phalange in digits 2 and 4. The synovial sheath of the tendons is carefully exposed and incised transversally to access the flexor digitorum profundus distal to the flexor digitorum superficialis bifurcation. Tendon injury is performed by gently lifting the flexor digitorum profundus with curved forceps and incising transversally through half of its substance. The formulation containing the test drug is applied around the tendons in the sheath of one of the two digits randomly selected. The other digit is left untreated and is used as a control. The sheath is then repaired with 6-0 nylon suture. An immobilizing 6-0 nylon suture is inserted through the transverse metacarpal ligament into the tendon/sheath complex to immobilize the tendon and the sheath as a single unit to encourage adhesion formation. The wound is closed with 4-0 interrupted sutures. A bandage is applied around the hindpaw to further augment immobilization of the digits and ensure comfort and ambulation of the animals. The animals are recovered and returned to their cage.

Three weeks after surgery, the animals are anesthetized. After a skin incision, the tissue plane around the synovial sheath is dissected and the tendon—sheath complex harvested en block and transferred in 10% phosphate buffered formaldehyde for histopathology analysis. The animals are then euthanized. After paraffin embedding, serial 5-um thin cross-sections are cut every 2 mm through the sheath and tendon complex. Sections are stained with H&E and Movat's stains to evaluate adhesion growth. Each slide is digitized using a computer connected to a digital microscope camera (Nikon Micropublisher cooled camera). Morphometry analysis is then performed using image analysis software (ImagePro). Thickness and area of adhesion defined as the substance obliterating the synovial space are measured and compared between formulation-treated and control animals.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 40

### Assessment of Paclitaxel in the Inhibition of Cartilage Damage in the ACL Injured Hartley Guinea Pig Model of Osteoarthritis

The purpose of this study was to determine whether paclitaxel administered in a hyaluronic acid formulation can delay or prevent the development of osteoarthritis in guinea pig knees.

Surgical Procedures.

Male Hartley guinea pigs, at least 6 weeks old, were anaesthetised using 5% isoflurane in an enclosed chamber. The animals were weighed and then transferred to the surgical table where anaesthesia was maintained by nose cone with 2% isoflurane. The knee area on the both legs was shaved and knee width at the head of the femur was measured on both knees. The skin on the right knee was sterilized. A 20G needle was introduced in the knee joint using a medial approach and the anterior cruciate ligament was cut with the sharp end of the needle. This procedure was practiced in a preliminary experiment that showed that the anterior cruciate ligament could be sectioned reliably using this technique.

Two weeks after the initial procedure, the animals were anesthetized with isoflurane (5% induction −2% maintenance) and weighed. The knee area on both legs was shaved and knee width at the head of the femur was measured on both knees. The skin of the injured knee was sterilised. A 25G needle was introduced into the synovial cavity using a medial approach and 0.1 ml of the test formulation was injected. Injections were repeated weekly for a total of 5 injections. Sample size was N=12 for each formulation. Two doses of paclitaxel and control formulation were tested.

Ten weeks after injury, the animals were sacrificed by cardiac injection of 0.7 ml Euthanyl under deep anaesthesia (5% isoflurane) and weighed. A final knee measurement was taken. The skin over the knee area was removed without damaging subcutaneous tissues. The knee joints were then harvested en bloc and placed into a formaldehyde (37%)/acetic acid solution (5:1 ratio) for fixation. Samples were sent to an independent laboratory for the conduct of histological preparation of joints and assessment by a pathologist for signs of cartilage damage.

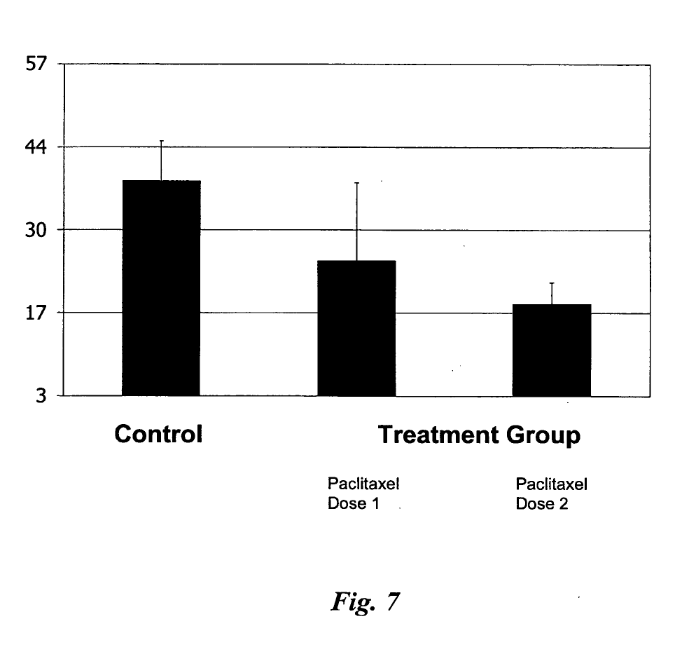

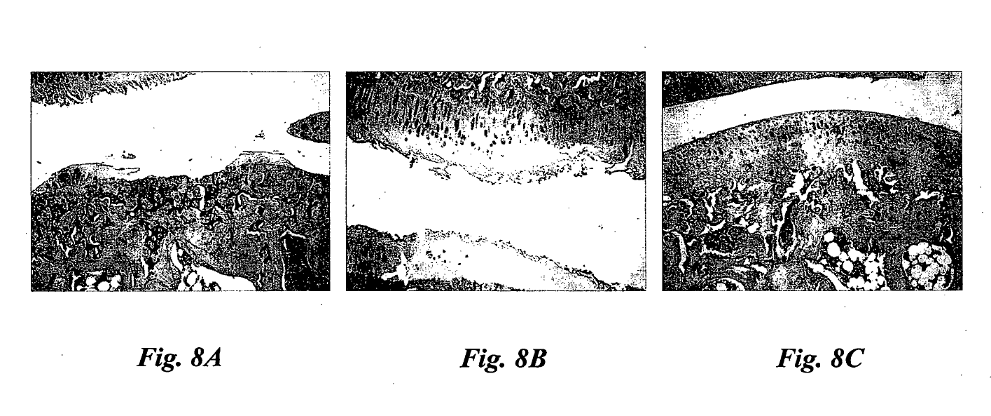

Briefly knee sections were made to examine cartilage and slides were stained with H&E stain. A pathologist scored slides in a blinded fashion from each animal using corresponding knee sections according to the following scale: no damage to cartilage, loss of proteoglycans, fraying of cartilage, loss of cartilage to the tidemark, and loss of cartilage to the bone. Bar graphs were constructed from each group and compared. Paclitaxel treatment at a low dose (dose 1) and medium dose (dose 2) showed a statistical reduction in cartilage damage relative to control. See FIGS. 7 and 8A-C.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 41

### Proteoglycan Loss Index in the Carrageenin-Induced and Antigen-Induced Rabbit Models of Arthritis Following Treatment with Paclitaxel

All microspheres were made using the oil in water solvent evaporation method described by Liggins and Burt (2001). The external phase was 100 ml of 1-5% PVA in water. The internal phase was 10 ml of a dichloromethane solution containing 5% w/v total solids (polymer and drug). The dispersion was stirred for 2 hours at room temperature to form microspheres. By varying the stirring speed between 900 and 2100 rpm and the PVA concentration, various size ranges were produced. The microspheres were separated from the external phase and rinsed with distilled water. Some microspheres were further divided into discrete size ranges by sieving the microspheres suspension through sieves having mesh sizes of 38, 53, 75 and 106 μm. Microsphere size distributions were determined using a Coulter LS130 particle size analyzer. Microspheres were suspended in water with a small amount of Tween 80 to prevent aggregation prior to particles size analysis. Chitosan microparticle size ranges were determined by optical microscopy using a microscope slide marked with 5 μm gradations. Optical microscopy was performed on both dry and wetted samples.

Thermal properties of the microspheres were determined using a Dupont Thermal Analysis DSC. Approximately 5 mg of microspheres were placed in unsealed aluminum pans and thermograms were obtained at a heating rate of 10° C./min. Evidence of crystallinity was obtained by X-ray powder diffraction measurements using a Rigaku X-ray diffractometer. Samples were scanned with a CuKα X-ray source through 5-35°2θ at a rate of 1°2θ/min with a step increment of 0.02°2θ.

The surface morphology of microspheres was determined using a Hitachi scanning electron microscope. Microspheres were coated with a 100 Å gold-palladium coat and visualized at a magnification of 1000×.

The drug content and in vitro release from microspheres were determined using the methods similar to that of Liggins & Burt (2001). For total content analysis, approximately 5 mg (accurately weighed) of microspheres were dissolved in 1 ml of dichloromethane followed by vigorous mixing with 15 ml of 60:40 acetonitrile:water. The solvent mixture was allowed to separate into two approximately equal volumes with a precipitated mass of polymer between the two. The amount of paclitaxel in each of the two fractions was then determined by HPLC using a Waters HPLC system.

Antigen induced arthritis was reproduced in rabbits using a previously described method (Kim et al., J. Rheumatol 1995:22:1714-21.). Briefly, female New Zealand white rabbits weighing 2.5-2.8 kg were used in biocompatibility and efficacy studies. Animals were housed in suspended caging with free access to food and water. Animals were acclimated for seven days prior to all experiments. Arthritis was induced in some animals for use as positive controls in biocompatibility testing and for use in efficacy studies. All knee joint injections were carried out under anaethesia induced by intramuscular injection of ketamine HCl (40 mg/kg) and xylazine (5 mg/kg). At the end of the in-life portion of the study, animals were sacrificed using intravenous T-61. The knee joints were dissected immediately after sacrifice and fixed in 10% formalin prior to histological analysis.

Antigen induced arthritis was established by three injections of bovine serum albumin (BSA) in Freund's complete adjuvant (FCA). The first injection consisted of 5 mg BSA emulsified in 1 ml FCA and diluted in 1 ml PBS. Three weeks later, each rabbit received a subcutaneous booster injection of 2.5 mg of BSA emulsified in 1 ml FCA diluted with 1 ml PBS. After four weeks, each rabbit received a second booster of 0.5 mg BSA in 0.3 mL pyrogen-free PBS injected into the knee joint. Five days after the final booster, the rabbits were treated by intra-articular injection with test articles.

Carrageenan induced arthritis was established in rabbits and the rabbits were treated in the same manner as for the antigen induced arthritis model. All rabbits in the carrageenan groups were injected with 0.3 ml of 1% carrageenan in pyrogen free PBS on days 1, 3, 8, 16 and 21. Half the animals were also injected with 35 mg of 20% drug-loaded microspheres on day 6. All animals were sacrificed on day 29 and the joints were dissected for histological analysis.

Synovial inflammation was assessed after sacrificing the rabbits. The joints were fixed in formalin and decalcified in 10% formic acid with repeated changes. The decalcified joints were embedded in paraffin and sections containing synovium, cartilage and bone were prepared. Sections were stained for cellularity with hematoxylin and eosin (H&E) and for proteoglycan content with safranin O. Synovial inflammation and cartilage degradation were evaluated by blinded histological evaluation of parapatellar synovium and femoral condylar articular cartilage, respectively. Villus hyperplasia, fibroblast proliferation, fibrosis, angiogenesis, mononuclear cell and polymorphonuclear cell infiltrations were graded as indicators of synovial inflammation. For cartilage degradation, surface erosion, proteoglycan content and chondrocyte necrosis were graded. Grading of cellular infiltration and swelling was scored with an integer from 0 to 4 based on increasing erythema, swelling and cellular infiltration (0, normal; 4, maximum). For slight effects, a score of 0.5 was assigned; this was the only non-integer score used. Proteoglycan loss was also scored from 0 (normal) to 4 (almost total loss of stained proteoglycans).

The efficacy of drug-loaded polyester microspheres given by intra-articular injection in treating antigen-induced arthritis was assessed using control and 20% loaded 10-35 and 35-105 μm PLA microspheres. Groups of five rabbits were treated with 40 mg of microspheres or PBS alone in the right joint. The left joint received PBS alone. The animals were sacrificed fourteen days after treatment and examined histologically for synovial inflammation and cartilage degradation as described above.

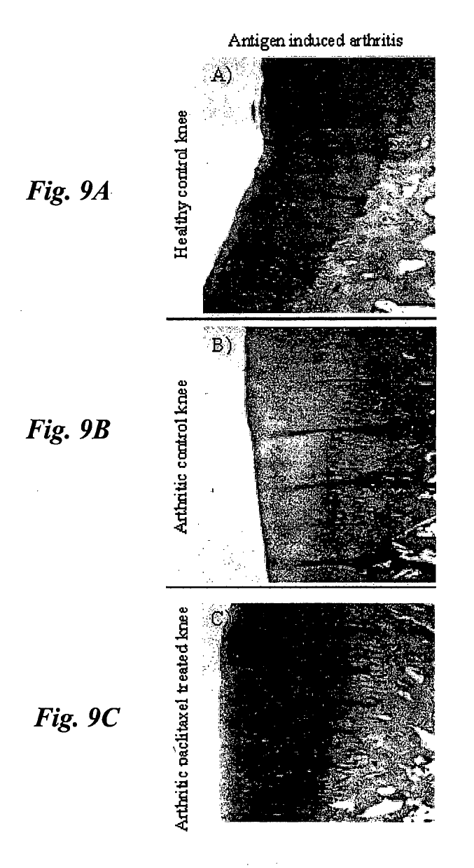

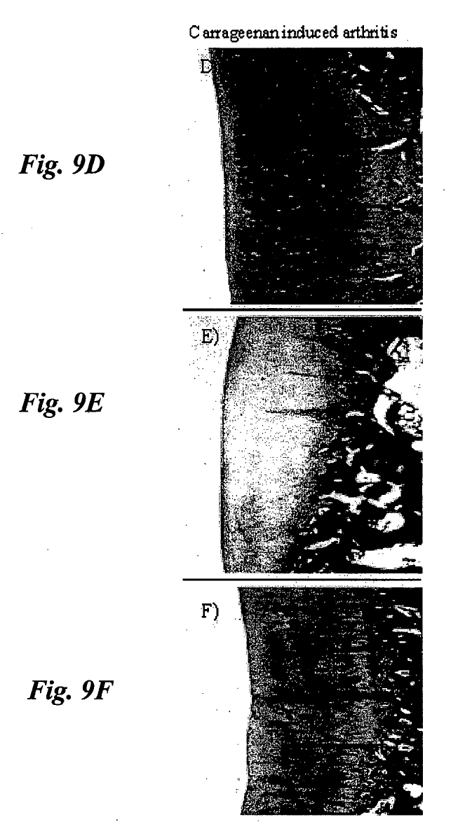

PLA microspheres containing 20% paclitaxel were selected for the efficacy study. The table below shows the results of the injection of 40 mg of control and paclitaxel-loaded PLA microspheres in rabbits with antigen induced arthritis. Untreated arthritic rabbits had a joint swelling score of 3 and 4.9×10⁷ cells in the joint fluid. Paclitaxel-loaded microspheres in the 10-35 um size range did not reduce antigen induced arthritis. In fact, the amount of cellular infiltration was elevated in this group relative to untreated arthritic rabbits (Table 1). However, the injection of 35-105 μm paclitaxel-loaded microspheres significantly reduced both the joint swelling and the number of cells in the joint fluid (about a 50% decrease) relative to control (Table 1). Cartilage degradation expressed as proteoglycan loss and chondrocyte necrosis was also assessed in the control groups and the paclitaxel-loaded 35-105 μm microspheres group. There was no effect on either proteoglycan loss or chondrocyte necrosis by the injection of control PLA microspheres in diseased animals. However, animals treated with paclitaxel-loaded microspheres had significantly less proteoglycan loss than the untreated animals (Table 1 and FIGS. 9A-9C). FIG. 9A illustrates a knee having a normal histological appearance, with a continuous top layer of cartilage and no loss of stain color indicating normal proteoglycan content (score 0). FIG. 9B shows a control microspheres arthritic knee with proteoglycan loss down to the bottom third layer of the section, which is termed heavy loss (score 3). In FIG. 9C, a paclitaxel microspheres treated arthritic knee shows only slight loss of proteoglycan at the surface layer of cartilage, with an intact surface (score 1).

The effect of paclitaxel-loaded microspheres in preventing proteoglycan loss in carrageenan-induced arthritis was not as prominent as in antigen induced arthritis (FIGS. 9D-F). FIG. 9E shows severe loss of proteoglycan throughout all layers of cartilage, but the surface layer remained intact (score 4). Treatment of carrageenan-induced knees with paclitaxel microspheres resulted in less reduction of stain color (FIG. 9F, score 2), but the protective effect was not as pronounced as observed in the antigen induced model (FIG. 9C).

Antigen induced arthritis was used to determine efficacy in these studies. Although this animal model takes some time to develop, it mirrors many aspects of human rheumatoid arthritis such as the production of inflammatory cytokines (such as TNF-α), the loss of proteoglycans and the infiltration of white blood cells into the joint with chronic inflammation. Results from this model are compared to those from carrageenan-induced arthritis which is quick to establish in the rabbits and offers a method of inducing intense and reproducible levels of acute (rather than chronic) forms of arthritis. Because carrageenan-induced arthritis is characterized by severe proteoglycan loss, this model was also used in this study to measure the effect of intraarticular paclitaxel on proteoglycan loss. Efficacy studies that included measurements of joint swelling, cell infiltration, proteoglycan loss and chondrocyte necrosis demonstrated that the single injection of 40 mg of 20% paclitaxel-loaded, 35-105 μm microspheres significantly reduced all aspects of the chronic arthritic condition in rabbits (Table 1 and FIGS. 9A-C). The effect of paclitaxel-loaded microspheres in preventing proteoglycan loss in the carrageenan induced arthritis model was not as pronounced as for the antigen induced arthritis model.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

TABLE 13
EFFICACY OF 40 MG OF CONTROL AND 20%
PACLITAXEL-LOADED PLA MICROSPHERES IN THE
SIZE RANGES OF 10-35 AND 35-105 MM, ASSESSED
IN TERMS OF MEAN SCORES (N = 5) FOR SWELLING,
CELLULAR INFILTRATION, LOSS OF PROTEOGLYCAN AND
CHONDROCYTE NECROSIS

| | Swelling | Number of | | Chondrocyte |
|---|---|---|---|---|
| | score | cell in joint | Proteoglycan | necrosis |
| Treatment | (0-4) | fluid | loss (0-4) | (0-3) |
| healthy, untreated | 0 | 7.0 × 10⁵ | Not tested | Not tested |
| control | | | | |
| 35-105 μm, | 3 | 4.9 × 10⁷ | 2 ± 0.6 | 1 ± 0.3 |
| control | | | | |
| 10-35 μm, 20% | 3 | 8.4 × 10⁷ | Not tested | Not tested |
| paclitaxel | | | | |
| 35-105 μm, 20% | 1 | 2.4 × 10⁷ | 1 ± 0.3 | 0 ± 0.1 |
| paclitaxel | | | | |

### Example 42

### Spinal Surgical Adhesions Model to Assess Fibrosis Inhibiting Drug Combinations or Individual Components Thereof in Rabbits

Extensive scar formation and adhesions often occur after lumbar spine surgery involving the vertebrae. The dense and thick fibrous tissue adherent to the spine and adjacent muscles must be removed by surgery. Unfortunately, fibrous adhesions usually reform after the secondary surgery. Adhesions are formed by proliferation and migration of fibroblasts from the surrounding tissue at the site of surgery. These cells are responsible for the healing response after tissue injury. Once they have migrated to the wound they lay down proteins such as collagen to repair the injured tissue. Overproliferation and secretion by these cells induce local obstruction, compression and contraction of the surrounding tissues with accompanying side effects.

The rabbit laminectomy spinal adhesion model described herein is used to investigate spinal adhesion prevention by local slow release of antifibrotic drugs.

Five to six animals are included in each experimental group to allow for meaningful statistical analysis. Formulations with various concentrations of antifibrotic drugs are tested against control animals to assess inhibition of adhesion formation.

Rabbits are anesthetized with an IM injection of ketamine/zylazine. An endotracheal tube is inserted for maintenance of anesthesia with halothane. The animal is placed prone on the operating table on top of a heating pad and the skin over the lower half of the back is shaved and prepared for sterile surgery. A longitudinal midline skin incision is made from L-1 to L-5 and down the lumbosacral fascia. The fascia is incised to expose the tips of the spinous processes. The paraspinous muscles are dissected and retracted from the spinous process and lamina of L-4. A laminectomy is performed at L-4 by removal of the spinal process with careful bilateral excision of the laminae, thus creating a small 5×10 mm laminectomy defect. Hemostasis is obtained with Gelfoam. The test formulations are applied to the injury site and the wound is closed in layers with Vicryl sutures. The animals are placed in an incubator until recovery from anesthesia and then returned to their cage.

Two weeks after surgery, the animals are anesthetized using procedures similar to those described above. The animals are euthanized with Euthanyl. After a skin incision, the laminectomy site is analyzed by dissection and the amount of adhesion is scored using scoring systems published in the scientific literature for this type of injury.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 43

### Tendon Surgical Adhesions Model to Assess Fibrosis Inhibiting Drug Combinations or Individual Components Thereof in Rabbits

This model is used to investigate whether adhesion of the tendons can be prevented by local slow release of drugs known to inhibit fibrosis. Polymeric formulations are loaded with drugs and implanted around injured tendons in rabbits. In animals without fibrosis -inhibiting formulations, adhesions develop within 3 weeks of flexor tendon injury if immobilization of the tendon is maintained during that period. An advantage of rabbits is that their tendon anatomy and cellular behaviour during tendon healing are similar to those in man except for the rate of healing that is much faster in rabbits.

Rabbits are anesthetized and the skin over the right hindlimb is shaved and prepared for sterile surgery. Sterile surgery is performed aided by an operating microscope. A longitudinal midline skin incision is made on the volvar aspect of the proximal phalange in digits 2 and 4. The synovial sheath of the tendons is carefully exposed and incised transversally to access the flexor digitorum profundus distal to the flexor digitorum superficialis bifurcation. Tendon injury is performed by gently lifting the flexor digitorum profundus with curved forceps and incising transversally through half of its substance. The formulation containing the test drug formulation is applied around the tendons in the sheath of one of the two digits randomly selected. The other digit is left untreated and is used as a control. The sheath is then repaired with 6-0 nylon suture. An immobilizing 6-0 nylon suture is inserted through the transverse metacarpal ligament into the tendon/sheath complex to immobilize the tendon and the sheath as a single unit to encourage adhesion formation. The wound is closed with 4-0 interrupted sutures. A bandage is applied around the hindpaw to further augment immobilization of the digits and ensure comfort and ambulation of the animals. The animals are recovered and returned to their cage.

Three weeks after surgery, the animals are anesthetized. After a skin incision, the tissue plane around the synovial sheath is dissected and the tendon—sheath complex harvested en block and transferred in 10% phosphate buffered formaldehyde for histopathology analysis. The animals are then euthanized. After paraffin embedding, serial 5-um thin cross-sections are cut every 2 mm through the sheath and tendon complex. Sections are stained with H&E and Movat's stains to evaluate adhesion growth. Each slide is digitized using a computer connected to a digital microscope camera (Nikon Micropublisher cooled camera). Morphometry analysis is then performed using image analysis software (ImagePro). Thickness and area of adhesion defined as the substance obliterating the synovial space are measured and compared between formulation-treated and control animals.

Exemplary drug combinations or their individual components that may be tested in this model include but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 44

### Parylene Coating

The metallic portion of a coronary stent is washed by dipping it into HPLC grade isopropanol. The cleaned device is then coated with a parylene coating using a parylene coater and either di-p-xylylene or dichloro-di-p-xylylene as the coating feed material. This procedure may be used to coat other types of medical devices that include a metallic portion (e.g., peripheral stents, covered stents, guidewires, shunts, GI drainage tubes, and anastomotic connectors).

### Example 45

### Drug Combination Coating—End Coating

Drug combination (amoxapine and prednisolone) solutions are prepared by dissolving amoxapine and prednisolone in 5 mL HPLC grade THF. The ends of a parylene coated coronary stent (prepared as in Example 44) are then dipped into the paclitaxel/THF solution. After various incubation times, the devices are removed and dried in a forced air oven (50° C.). The device is then further dried in a vacuum oven overnight. The amount of the drug combination (amoxapine and prednisolone) used in each solution is varied such that the amount of paclitaxel coated onto the ends of the device is in the range of 0.06 mg/mm² to 10 mg/mm².

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of devices that include a metallic portion (e.g., peripheral stents, covered stents, guidewires, GI drainage tubes, shunts, and anastomotic connectors).

### Example 46

### Drug Combination Coating—Complete Coating

Drug combination (amoxapine and prednisolone) solutions are prepared by dissolving paclitaxel in 5 mL HPLC grade THF. A parylene coated coronary stent (as prepared in Example 44) is then dipped entirely into the drug combination/THF solution. After various incubation times, the device is removed and dried in a forced air oven (50° C.). The device is then further dried in a vacuum oven overnight. The amount of the drug combination (amoxapine and prednisolone) used in each solution is varied such that the amount of paclitaxel coated onto the ends of the device is in the range of 0.06 mg/mm² to 10 mg/mm².

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of parylene coated devices that include a metallic portion (e.g., peripheral stents, covered stents, guidewires, GI drainage tubes, shunts, and anastomotic connectors).

### Example 47

### Application of a Parylene Overcoat

A combination of amoxapine and prednisolone coated device is placed in a parylene coater and an additional thin layer of parylene is deposited on the tersirolimus coated device (see Examples 2 or 3). The coating duration is altered such that the parylene top-coat thickness is varied such that different elution profiles of the drug combination of amoxapine and prednisolone may be obtained.

### Example 48

### Application of an Echogenic Coating Layer

DESMODUR (Bayer AG, Germany), an isocyanate pre-polymer, is dissolved in a 50:50 mixture of dimethylsulfoxide and tetrahydrofuran. A drug combination of amoxapine and prednisolone/parylene overcoated coronary stent (prepared as in Example 47) is then dipped into the pre-polymer solution. The device is then removed and the coating is then partially dried at room temperature for 3 to 5 minutes. The device is then immersed in a beaker of water (room temperature) for 3-5 minutes to cause the polymerization reaction to occur rapidly. An echogenic coating is formed. This procedure may be used to coat other types of devices (e.g., peripheral stents, covered stents, guidewires, GI drainage tubes, shunts, and anastomotic connectors).

### Example 49

### Drug Combination/Polymer Coating—End Coating

5% solutions of poly(ethylene-co-vinyl acetate) (EVA) (60% vinyl acetate) are prepared using THF as the solvent. Various amounts of a drug combination of amoxapine and prednisolone are added to each of the EVA solutions. The ends of a corornary stent are dipped into the drug combination of amoxapine and prednisolone /EVA solution. After removing the end-coated device from the solution, the coating is dried by placing the device in a forced air oven (40° C.) for 3 hours. The coated device is then further dried under vacuum for 24 hours. The dip coating process may be repeated to increase the amount of polymer/drug combination coated onto the device.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of devices (e.g., central venous catheters, ventricular assist devices, peripheral stents, and nasal stents).

### Example 50

### Drug Combination-Heparin Coating—End Coating

5% solutions of poly(ethylene-co-vinyl acetate) (EVA) (60% vinyl acetate) are prepared using THF as the solvent. Various amounts of a drug combination of amoxapine and prednisolone and a solution of tridodecyl methyl ammonium chloride-heparin complex (PolySciences) are added to each of the EVA solutions. the ends of an anastomotic connector device are dipped into the drug combination of amoxapine and porednisolone/EVA solution. After removing the end-coated device from the solution, the coating is dried by placing the anastomotic device in a forced air oven (40° C.) for 3 hours. The coated anastomotic device is then further dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of devices including peritoneal dialysis catheters, coronary stents, peripheral stents, hemodialysis access devices, guidewires, shunts, and VAD's.

### Example 51

### Drug Combination—Heparin/Heparin Coating

The uncoated portions of drug combination of amoxapine and prednisolone-heparin coated devices (Example 50) are dipped into a 5% EVA solution containing different amounts of a tridodecyl methyl ammonium chloride-heparin complex solution (PolySciences). After removing the end-coated device from the solution, the coating is dried by placing the anastomotic device in a forced air oven (40° C.) for 3 hours. The coated device is then further dried under vacuum for 24 hours. This provides a device with a drug combination of amoxapine and prednisolone/heparin coating on the ends of the device and a heparin coating on the remaining parts of the device.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coated other types of devices including peritoneal dialysis catheters, coronary stents, peripheral stents, hemodialysis access devices, guidewires, shunts, and VAD's

### Example 52

### Drug Combination/Polymer Coating—End Coating

5% solutions of poly(styrene-co-isobutylene-styrene) (SIBS) are prepared using THF as the solvent. Various amounts of a drug combination of amoxapine and prednisolone are added to each of the SIBS solutions. The ends of a central venous catheter device are dipped into the drug combination of amoxapine and prednisolone/SIBS solution. After removing the end-coated device from the solution, the coating is dried by placing the device in a forced air oven (40° C.) for 3 hours. The coated device is then further dried under vacuum for 24 hours. The dip coating process may be repeated to increase the amount of polymer/drug combination coated onto the device.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of devices including peritoneal dialysis catheters, coronary stents, non-vascular stents, peripheral stents, hemodialysis access devices, guidewires, shunts, and anastomotic connectors, LVAD'S.

### Example 53

### Drug Combination/Polymer Coating—Echogenic Overcoat

A coated CVC device from Example 52 is dipped into a DESMODUR solution (50:50 mixture of dimethylsulfoxide and tetrahydrofuran). The anastomotic device is then removed and the coating is then partially dried at room temperature for 3 to 5 minutes. The device is then immersed in a beaker of water (room temperature) for 3-5 minutes to cause the polymerization reaction to occur rapidly. An echogenic coating is formed.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 54

### Polymer/Echogenic Coating

5% solutions of poly(styrene-co-isobutylene-styrene) (SIBS) are prepared using THF as the solvent. A LVAD device is dipped into the SIBS solution. After removing the device from the solution, the coating is dried by placing the device in a forced air oven (40° C.) for 3 hours. The coated device is then further dried under vacuum for 24 hours.

The coated device is dipped into a DESMODUR solution (50:50 mixture of dimethylsulfoxide and tetrahydrofuran). The device is then removed and the coating is then partially dried at room temperature for 3 to 5 minutes. The device is then immersed in a beaker of water (room temperature) for 3-5 minutes to cause the polymerization reaction to occur rapidly. The device is dried under vacuum for 24 hours at room temperature. The ends of the coated device are immersed into a solution of a drug combination of amoxapine and prednisolone. The device is removed and dried at 40° C. for 1 hour and then under vacuum for 24 hours.

The amount of the drug combination of ampoxapine and prednisolone absorbed by the polymeric coating may be altered by changing the concentration of the drug combination of ampoxapine and prednisolone, the immersion time as well as the solvent composition of the drug combination solution.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of devices including peritoneal dialysis catheters, coronary stents, non-vascular stents, peripheral stents, hemodialysis access devices, guidewires, shunts, anastomotic connectors, CVC's.

### Example 55

### Combination/Siloxane Coating—End Coating

A central venous catheter is coated with a silioxane layer by exposing the device to gaseous tetramethylcyclotetrasiloxane that is then polymerized by low energy plasma polymerization onto the device surface. The thickness of the siloxane layer may be increased by increasing the polymerization time. The ends of the device are then immersed into a drug combination of amoxapine and prednisolone/THF solution. The drug combination of amoxapine and prednisolone is absorbed into the siloxane coating. The device is then removed from the solution and is dried for 2 hours at 40° C. in a forced air oven. The device is then further dried under vacuum at room temperature for 24 hours. The amount of the drug combination of amoxapine and prednisolone coated onto the device ends may be varied by altering the concentration of the drug combination/THF solution as well as altering the immersion time of the device ends in the drug combination/THF solution.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

This procedure may be used to coat other types of devices including peritoneal dialysis catheters, coronary stents, non-vascular stents, peripheral stents, hemodialysis access devices, guidewires, GI drainage tubes, shunts, and anastomotic connectors.

### Example 56

### Heparin Coating

A CNS shunt device is dipped into a solution containing different amounts of a tridodecyl methyl ammonium chloride-heparin complex solution (PolySciences). After various incubation times, the device is removed and dried in a forced air oven (50° C.). The device is then further dried in a vacuum oven overnight. Other types of devices that may be coated with this procedure include coronary stents, peripheral stents, nasal and sinus stents, tracheal stents, peritoneal dialysis catheters, vascular grafts, hemodialysis access devices, guidewires, shunts, and anastomotic connectors.

### Example 57

### Spray-Coated Devices

2% solutions poly(styrene-co-isobutylene-styrene) (SIBS) are prepared using THF as the solvent. Various amounts of a drug combination of amoxapine and prednisolone are added to each solution. A device (e.g., a stent, central venous catheter, LVAD, anastomotic connector, or shunt) is held with a pair of tweezers and is then spray coated with one of the drug combination of amoxapine and prednisolone/polymer solutions using an airbrush. The device is then air-dried. The device is then held in a new location using the tweezers and a second coat of drug combination of amoxapine and prednisolone/polymer is applied. The device is air-dried and is then dried under vacuum overnight. The total amount of the drug combination of amoxapine and prednisolone coated onto the device may be altered by changing the drug combination content in the solution as well as by increasing the number of coatings applied.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 58

### Drug Coated Covered Stent-Non-Degradable

A covered stent (WALLGRAFT, Boston Scientific Corporation) is attached to a rotating mandrel. A solution of a drug combination of amoxapine and prednisolone (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 59

### Drug Combination Coated Covered Stent—Degradable

A WALLGRAFT stent is attached to a rotating mandrel. A drug combination of amoxapine and prednisolone (5% w/w) in a PLGA/ethyl acetate solution (2.5% w/v) is then sprayed onto the outer surface of the covered stent. The solution is sprayed on at a rate that ensures that the graft material is not damaged or saturated with the sprayed solution. The covered stent is allowed to air dry after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 60

### Drug Combination Coated Covered Stent—Degradable Overcoat

A drug combination-coated WALLGRAFT stent from either Example 58 or Example 59 is attached to a rotating mandrel. A PLGA/ethyl acetate solution (2.5% w/v) is then sprayed onto the outer surface of the covered stent such that a coating is formed over the initial drug containing coating. The solution is sprayed on at a rate that ensures that the graft material is not damaged or saturated with the sprayed solution. The covered stent is allowed to air dry after which it is dried under vacuum for 24 hours.

### Example 61

### Drug Combination-Loaded Microsphere Formulation

A drug combination of amoxapine and prednisolone (10% w/w) is added to a solution of PLGA (50/50, Mw≈54,000) in DCM (5% w/v). The solution is vortexed and then poured into a stirred (overhead stirrer with a 3 bladed TEFLON coated stirrer) aqueous PVA (approximately 89% hydrolyzed, Mw≈13,000, 2% w/v). The solution is stirred for 6 hours after which the solution is centrifuged to sediment the microspheres. The microspheres were resuspended in water. The centrifugation—washing process is repeated 4 times. The final microsphere solution is flash frozen in an acetone/dry-ice bath. The frozen solution is then freeze-dried to produce a fine powder. The size of the microspheres formed may be altered by changing the stirring speed and/or the PVA solution concentration. The freeze dried powder may be resuspended in PBS or saline and may be used for direct injection, as an incubation fluid or as an irrigation fluid.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 62

### Drug Combination Coated Stent (Exterior Coating)

A stent is dipped into a polyurethane (CHRONOFLEX 85A)/THF solution (2.5% w/v). The coated stent is allowed to air dry for 10 seconds. The stent is then rolled in powdered drug combination of amoxapine and prednisolone that is spread thinly on a piece of release liner. The rolling process is done in such a manner that the powder of the drug combination of amoxapine and prednisolone predominantly adheres to the exterior side of the coated stent. The stents are air-dried for 1 hour followed by vacuum drying for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 63

### Drug Combination Coated Stent (Exterior Coating) with a Heparin Coating

The drug combination-coated stent from Example 62 is further coated with a heparin coating. The stents that are prepared in Example 62 are dipped into a solution of heparin-benzalkonium chloride complex (1.5% (w/v) in isopropanol, STS Biopolymers). The stents are removed from the solution and are air-dried for 1 hour followed by vacuum drying for 24 hours. This process results in both the interior and exterior surfaces of the covered stent being coated with heparin.

### Example 64

### Partial Drug Combination Coating of a Covered Stent

A WALLGRAFT covered stent is attached to a rotating mandrel. A mask system is set up so that only the middle of the outer surface of the covered stent may be sprayed. A solution of a drug combination of amoxapine and prednisolone (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 65

### Drug—Dexamethasone Coated Covered Stent

A WALLGRAFT covered stent is attached to a rotating mandrel. A mask system is set up so that only the middle of the outer surface of the covered stent may be sprayed. A solution of a drug combination of amoxapine and prednisolone (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry. The mask is then rearranged so that only the ends of the outer surface of the covered stent may be sprayed. The ends of the outer surface of the covered stent are then sprayed with a dexamethasone (10% w/w)/polyurethane (CHRONOFLEX 85A)/THF solution (2.5% w/v). The sample is air dried after which it is dried under vacuum for 24 hours. microspheres in preventing proteoglycan loss in the carrageenan induced arthritis

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 66

### Drug Combination—Heparin Coated Covered Stent

A WALLGRAFT covered stent is attached to a rotating mandrel. A mask system is set up so that only the middle of the outer surface of the covered stent may be sprayed. A solution of a drug combination of amoxapine and prednisolone (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry. The mask is then rearranged so that only the ends of the outer surface of the covered stent may be sprayed. The ends of the outer surface of the covered stent are then sprayed with a heparin-benzalkonium chloride complex (1.5% (w/v) in isopropanol, STS Biopolymers). The sample is air dried after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 67

### Drug Combination—Dexamethaxone Coated Covered Stent

A WALLGRAFT stent is attached to a rotating mandrel. A solution of a drug combination of amoxapine and prednisolone (5% w/w) and dexamethazone (5%w/w) in a PLGA (50/50, Mw≈54,000)/ethyl acetate solution (2.5% w/v) is sprayed onto the outer surface of the covered stent. The solution is sprayed on at a rate that ensures that the graft material is not damaged or saturated with the sprayed solution. The covered stent is allowed to air dry after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 68

### Drug Combination—Dexamethasone Coated Covered Stent (Sequential Coating)

A WALLGRAFT stent is attached to a rotating mandrel. A solution of a drug combination of amoxapine and prednisolone (5% w/w) in a PLGA (50/50, Mw≈54,000)/ethyl acetate solution (2.5% w/v) is sprayed onto the outer surface of the covered stent. The solution is sprayed on at a rate that ensures that the graft material is not damaged or saturated with the sprayed solution. The covered stent is allowed to air dry. A methanol solution of dexamethasone is then sprayed onto the outer surface of the covered stent (at a rate that ensures that the graft material is not damaged or saturated with the sprayed solution). The covered stent is allowed to air dry after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 69

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1L HPLC grade water is added and the solution is stirred until all the salts are dissolved. If required, the pH of the solution is adjusted to pH 7.4±0.2 using either 0.1N NaOH or 0.1N phosphoric acid.

### Example 70

### Release Study to Determine Release Profile of the Drug Combination or Individual Components Thereof From a Coated Device

A sample of the drug combination-loaded catheter is placed in a 15 ml culture tube. 15 ml release buffer (Example 69) is added to the culture tube. The tube is sealed with a TEFLON lined screw cap and is placed on a rotating wheel in a 37° C. oven.

At various time points, the buffer is withdrawn from the culture tube and is replaced with fresh buffer. The withdrawn buffer is then analyzed for the amount of the drug combination or individual components thereof contained in this buffer solution using HPLC.

### Example 71

### Perivascular Administration of Drug Combination of Amoxapine and Prednisolone

WISTAR rats weighing 250-300 g are anesthetized by the intramuscular injection of Innovar (0.33 ml/kg). Once sedated, they are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 FRENCH FOGART balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

A 0.8 cm×0.8 cm piece of a drug combination material (as prepared in Examples 85 and 86) is then injected in a circumferential fashion around the common carotid artery in ten rats. EVA alone is injected around the common carotid artery in ten additional rats. (The drug combination may also be coated onto an EVA film which is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

### Example 72

### Complete Coating—Dip Coating a Vena Cava Filter

Poly(ethylene-co-vinyl acetate) {28% vinyl acetate} [p(EVA)] is dissolved in 10 ml THF to produce a solution that has a polymer concentration of approximately 40 mg/mL. A drug combination of amoxapine and predenisolone is added to the pEVA solution to produce a final drug combination concentration of 3 mg/mL. A vena cava filter is cleaned by immersing the filter into isopropanol for 30 minutes and then rinsing 3 times with isopropanol. The filter is air dried. The filter is dip coated by completely immersing the cleaned filter into the pEVA-drug combination solution. The filter is the removed from the solution and is air dried. This process may be repeated until the desired dose of the drug combination is achieved. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 73

### Partial Coating—Dip Coating a Vena Cava Filter

Polyurethane (CHRONOFLEX AL 85A) is dissolved in 10 ml THF to produce a solution that has a polymer concentration of approximately 400 mg/mL. A drug combination of amoxapine and prednisolone is added to the polyurethane solution to produce a final everolimus concentration of 3 mg/mL. A vena cava filter is cleaned by immersing the filter into isopropanol for 30 minutes and then rinsing 3 times with isopropanol. The filter is air dried. The filter is dip coated by immersing only the portions of the cleaned filter that will come into contact with the body tissue into the polyurethane-drug combination solution. The filter is the removed from the solution and is air dried. This process may be repeated until the desired everolimus dose is achieved. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 74

### Complete Coating—Spray Coating

A 2% solution poly(styrene-co-isobutylene-styrene) (SIBS) is prepared using THF as the solvent. A drug combination of amoxapine and prednisolone is added to the SIBS solution to produce a final drug combination concentration of 3 mg/mL. The SIBS-drug combination solution is then transferred to the reservoir of an artist's air brush tool. A vena cava filter is cleaned by immersing the filter into isopropanol for 30 minutes and then rinsing 3 times with isopropanol. The filter is air dried. Using a crocodile clip, the filter is suspended in the air and is spray coated from several angles to ensure complete coating of the filter. Once the coating is dry to the touch, the filter is removed from the clip and the uncoated portion is spray coated. The filter is then air dried and/or vacuum dried to remove the solvent. This process may be repeated until the desired drug combination dose is achieved. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 75

### Partial Coating—Spray Coating a Vena Cava Filter

A 2% solution poly(styrene-co-isobutylene-styrene) (SIBS) is prepared using THF as the solvent. A drug combination of amoxapine and prednisolone is added to the SIBS solution to produce a final concentration of 3 mg/mL. The SIBS-drug combination solution is then transferred to the reservoir of an artist's air brush tool. A vena cava filter is cleaned by immersing the filter into isopropanol for 30 minutes and then rinsing 3 times with isopropanol. The filter is air dried. Using a crocodile clip that is attached to a portion of the filter that is not to be coated, the filter is suspended in the air and is spray coated through a mask to ensure that only the desired portions of the filter are coated. The filter is then air dried and/or vacuum dried to remove the solvent. This process may be repeated until the desired drug combination dose is achieved. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 76

### Application of a Second Coating to a Vena Cava Filter

Poly(ethylene-co-vinyl acetate) {28% vinyl acetate) [p(EVA)] is dissolved in 10 ml THF to produce a solution that has a polymer concentration of approximately 40 mg/mL. A drug combination of amoxapine and prednisolone is added to the pEVA solution to produce a final drug combination concentration of 3 mg/mL. A vena cava filter is cleaned by immersing the filter into isopropanol for 30 minutes and then rinsing 3 times with isopropanol. The filter is air dried. The filter is dip coated by completely immersing the cleaned filter into the pEVA-drug combination solution. The filter is the removed from the solution and is air dried. This process may be repeated until the desired drug combination dose is achieved. The filter is then dried under vacuum to remove the residual solvent. The filter is then dipped into an aqueous solution of sodium hyaluronate [HA] (mw approximately 1-1.5×10⁶ kDa, 10 mg/mL). The water is removed by air drying at 37° C. The process is repeated until the desired amount of HA is coated onto the filter. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 77

### Coating Containing Two Bioactive Agents for a Vena Cava Filter

Poly(ethylene-co-vinyl acetate) {28% vinyl acetate) [p(EVA)] is dissolved in 10 ml THF to produce a solution that has a polymer concentration of approximately 40 mg/mL. A drug combination of amoxapine and prednisolone is added to the pEVA solution to produce a final drug combination concentration of 3 mg/mL. Heparin-benzalkonium chloride is then added to the pEVA solution to achieve a final concentration of 1 mg/ml. A vena cava filter is cleaned by immersing the filter into isopropanol for 30 minutes and then rinsing 3 times with isopropanol. The filter is air dried. The filter is dip coated by completely immersing the cleaned filter into the pEVA-drug combination solution. The filter is the removed from the solution and is air dried. This process may be repeated until the desired drug combination dose is achieved. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 78

### Two Coating Layers Containing Two Different Bioactive Agents for a Vena Cava Filter

Poly(ethylene-co-vinyl acetate) {28% vinyl acetate) [p(EVA)] is dissolved in 10 ml THF to produce a solution that has a polymer concentration of approximately 40 mg/mL. A drug combination of amoxapine and prednisone is added to the pEVA solution to produce a final drug combination concentration of 3 mg/mL. A vena cava filter is cleaned by immersing the filter into isopropanol for 30 minutes and then rinsing 3 times with isopropanol. The filter is air dried. The filter is dip coated by completely immersing the cleaned filter into the pEVA-drug combination solution. ** The filter is the removed from the solution and is air dried. This process may be repeated until the desired drug combination dose is achieved. The filter is then dried under vacuum to remove the residual solvent. The filter is then dipped into an aqueous solution of sodium hyaluronate [HA] (mw approximately 1-1.5×10⁶ kDa, 10 mg/mL) that contains 1 mg/ml heparin. The water is removed by air drying at 37° C. The process is repeated until the desired amount of HA is coated onto the filter. The filter is then dried under vacuum.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 79

### Drug Combination Incorporation into a Vascular Graft

A solution of a drug combination of amoxapine and prednisolone is prepared by dissolving 70 mg of the drug combination in 10 mL water/ethanol (1:1) in a 20 mL glass scintillation vial. A 5 cm piece of an ePTFE vascular graft (IMPRA, 6 mm) is immersed in the solution. The solution is placed in an ultrasonic bath (Fisher) for 1 min. The graft is removed using a pair of tweezers. The graft is air dried for 3 hours after which it is dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 80

### Drug Combination Incorporation into a Tympanostomy Tube

Five 15 mL solutions of a drug combination of amoxapine and prednisolone at 5 mg/ml are prepared in methanol in a 20 mL scintillation vial. A soft silicone T-tube ((Medco Catalogue Number T5030) is then immersed in each of the drug combination solutions. The tubes are removed from the drug combination solutions at 30 min, 1 hour, 2 hours, 6 hours and 24 hours. The tubes are air dried and then dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 81

### Drug Combination Incorporation into a Tympanostomy Tube

Five 15 mL solution of a drug combination of famoxapine and prednisolone (5 mg/mL) and 5-fluorouracil (4 mg/mL) are prepared in methanol in a 20 mL scintillation vial. A soft silicone T-tube ((Medco Catalogue Number T5030) is then immersed in each of the drug combination solutions. The tubes are removed from the drug combination solutions at 30 min, 1 hour, 2 hours, 6 hours and 24 hours. The tubes are air dried and then dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 82

### Drug Combination Incorporation into an Intraocular Lens

Five 15 mL solution of a drug combination of amoxapine and prednisolone (1 mg/mL) are prepared in methanol in a 20 mL scintillation vial. An intra-ocular lens (STAAR) then immersed in each of the drug combination solutions. The lenses are removed from the drug combination solutions at 30 min, 1 hour, 2 hours, 6 hours and 24 hours. The lenses are air dried and then dried under vacuum for 24 hours.

In addition to the drug combination of amoxapine and prednisolone, exemplary drug combinations that may be used to coat the device include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 83

### Synthesis of Polymer MePEG750-PDLLA-2080 Polymer

To synthesize the MePEG750-PDLLA-2080 polymer, 40 g of MePEG (molecular weight=750; Sigma-Aldrich, St. Louis, Mo.) was weighed in a 500 RB flask and 160 g of D,L-lactide (PURASORB®, PURAC, Lincolnshire, Ill.) was weighed in a weigh boat. Both reagents were dried under a vacuum overnight at room temperature. Then 600 mg stannous 2-ethyl-hexanoate catalyst (Sigma) was added into the round bottom flask containing the MePEG and a magnetic stir bar. The flask was purged with N₂ (oxygen free) for 5 minutes, capped with a glass stopper, placed into an oil-bath (maintained at 135° C.), and a magnetic stirrer was gradually turned onto setting 6 (Corning). After 30 minutes, the flask was removed from the oil-bath and was cooled to room temperature in a water bath. The D,L-lactide was added into the flask, which was then purged with oxygen free N₂ for 15 minutes, the flask was capped and again placed in the oil-bath (135° C.). The magnetic stirrer was turned on to a setting of 3 and the polymerization reaction was allowed to continue for at least five (5) hours. The flask was removed from the oil bath and the molten polymer poured into a glass container and allowed to cool to room temperature.

### Example 84

### Purification of MePEG750-PDLLA-2080

The MePEG750-PDLLA-2080 was prepared as outlined in Example 83, then 75 g MePEG750-PDLLA-2080 was dissolved in 100 ml of ethyl acetate (Fisher, HPLC grade) in a 250 ml conical flask. The polymer was precipitated by slowly adding the solution into 900 ml isopropanol (Caledon, HPLC grade) in a 2 L conical flask while stirring. The solution was stirred for 30 minutes and the suspension cooled to 5° C. using a cooling system. The supernatant was separated and the precipitant transferred to a 400 ml beaker. The polymer was first pre-dried in a forced-air oven at 50° C. for 24 hours to remove the bulk of the solvent. The pre-dried polymer was then transferred to a vacuum oven (50° C.) and further dried for 24 hours until the residual solvent was below an acceptable level. The purified polymer was stored at 2-8° C.

### Example 85

### Coating of MePEG750-PDLLA-2080 on a PLGA (10:90) Mesh with Amoxapine and Prednisolone

A PLGA (10/90) mesh of dimension 3×6 cm² is washed with isopropanol (Caledon, HPLC) and dried in a forced-air oven at 50° C. Then 3 g MePEG750-PDLLA-2080 is dissolved in 15 ml ethyl acetate (20% solution; Fisher HPLC grade) in a 20 mL glass scintillation vial. A drug combination of amoxapine (10 mg) and prednisolone (10 mg) is added to the polymer solution and the paclitaxel is completely dissolved by using a vortex mixer. A mesh is coated with the polymer/amoxapine and prednisolone solution by dipping into such a solution. The excess solution is then removed and the coated mesh is dried using an electric fan for 2-3 minutes. The coated mesh is placed in a PTFE petri-dish and is further dried for 60 minutes using the electric fan in a fume-hood. The coated mesh is then transferred into a vacuum oven and dried under vacuum overnight at room temperature. The dried coated mesh is packed between two pieces of release-liners (Rexam A10) and sealed in a pouch bag.

In addition to the drug combination of amoxapine and prednisolone, exemplary drug combinations that may be used to coat a mesh include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 86

### Electrospinning of Drug Combination-Loaded Material

10% solutions of PLGA (50:50, Mw≈54,000) are prepared by dissolving 1 g PLGA into 10 mL DCM. Various amounts of a drug combination of amoxapine and prednisolone are added to each solution such that the total drug percentage relative to the polymer ranges from 0.5% to 20%. Each solution is then loaded into a 10 ml syringe fitted with a 20 gauge needle. The syringe is then loaded into a syringe pump and 20 kV positive high voltage (by Glassman High Voltage, Inc., High Bridge, N.J.) is applied on the syringe needle. The grounded target drum is a rotating drum that has a diameter of about 12 cm. The syringe pump is set to pump at 25 ul per minute and the drum is rotated at approximately 250 rpm. The distance from the tip of the needle to the outside of the drum surface is about 14 cm. The rotating drum is moved from side to side during the spinning process such that the drum is virtually completely covered in the spun material. After the spinning process is completed, a razor blade is used to make a cut through the entire length of the spun material. The material is removed from the drum and is further dried in a vacuum oven for 24 hours.

In addition to the drug combination of amoxapine and prednisolone, exemplary drug combinations that may be used to form materials suitable for electrospinning include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 87

### Amoxapine and Prednisolone-Loaded PLG Microspheres (<10 Micron)

800 mg PLG (85:15, Absorbable Polymers International) is dissolved in 20 ml dichloromethane. A total of 160 mg of amoxapine (80 mg) and prednisolone (80 mg) is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

In addition to the drug combination of amoxapine and prednisolone, exemplary drug combinations that may be used to prepare drug combination-loaded PLG microspheres include but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 88

### Amoxapine and Prednisolone Containing Microspheres (50-100 Micron)

Microspheres having an average size of about 50-100 microns are prepared using a 1% PVA solution and 500 rpm stirring rate using the same procedure described in Example 87.

In addition to the drug combination of amoxapine and prednisolone, the above process may be used in preparing microspheres (50-100 Micron) that contain one of the following exemplary drug combinations, including but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, and itraconazole and lovastatin.

### Example 89

### Amoxapine-Loaded PLG Microspheres (<10 Micron)

800 mg PLG (85:15, Absorbable Polymers International) is dissolved in 20 ml dichloromethane. 125 mg amoxapine (Sigma, CAT# A129) is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg amoxapine.

### Example 90

### Prednisolone-Loaded PLG Microspheres (<10 Micron)

800 mg PLG (85:15, Absorbable Polymers International) is dissolved in 20 ml dichloromethane. 125 mg prednisolone (Sigma, Cat# P6004) is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg prednisolone.

### Example 91

### Mixtures of Amoxapine-Loaded PLG Microspheres and Prednisolone-Loaded PLG Microspheres (<10 Micron)

Mixtures of the amoxapine-loaded PLG microspheres and prednisolone-loaded PLG microspheres are prepared by weighing out specific amounts of the amoxapine-loaded PLG microspheres (as prepared in Example 89) and specific amounts of the prednisolone-loaded PLG microspheres (as prepared in Example 90) into a 6 ml glass scintillation vial (Sigma, Cat #M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added amoxapine-loaded PLG microspheres and added prednisolone-loaded PLG micro spheres is 200 mg. The ratio of amoxapine-loaded PLG micro spheres to prednisolone-loaded PLG microspheres is adjusted to give a range of amoxapine and prednisolone ratios. Specific ratios that are prepared include 75:25 (wt/wt), 60:40, 50:50, 40:60, 25:75 amoxapine:prednisolone.

### Example 92

### Paroxetine-Loaded PLG Microspheres (<10 Micron)

800 mg PLG (85:15, Absorbable Polymers International) is dissolved in 20 ml dichloromethane. 125 mg paroxetine is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg paroxetine.

### Example 93

### Prednisolone-Loaded PLG Microspheres (<10 Micron)

800 mg PLG (85:15, Absorbable Polymers International) is dissolved in 20 ml dichloromethane. 125 mg prednisolone is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg prednisolone.

### Example 94

### Mixtures of Paroxetine-Loaded PLG Microspheres and Prednisolone-Loaded PLG Microspheres (<10 Micron)

Mixtures of the paroxetine-loaded PLG microspheres and prednisolone-loaded PLG microspheres are prepared by weighing out specific amounts of the paroxetine-loaded PLG microspheres (as prepared in Example 92) and specific amounts of the prednisolone-loaded PLG microspheres (as prepared in Example 93) into a 6 ml glass scintillation vial (Sigma, Cat #M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added paroxetine-loaded PLG microspheres and added prednisolone-loaded PLG microspheres is 200 mg. The ratio of paroxetine-loaded PLG microspheres to prednisolone-loaded PLG microspheres is adjusted to give a range of paroxetine and prednisolone ratios. Specific ratios that are prepared include 75:25 (wt/wt), 60:40, 50:50, 40:60, 25:75 paroxetine:prednisolone.

### Example 95

### Dipyridamole-Loaded PLG Microspheres (<10 Micron)

800 mg PLG (85:15, Absorbable Polymers International) is dissolved in 20 ml dichloromethane. 125 mg dipyridamole is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg dipyridamole.

### Example 96

### Prednisolone-Loaded PLG Microspheres (<10 Micron)

800 mg PLG (85:15, Absorbable Polymers International) is dissolved in 20 ml dichloromethane. 125 mg prednisolone is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg prednisolone.

### Example 97

### Mixtures of Dipyridamole-Loaded PLG Microspheres and Prednisolone-Loaded PLG Microspheres (<10 Micron)

Mixtures of the dipyridamole-loaded PLG microspheres and prednisolone-loaded PLG microspheres are prepared by weighing out specific amounts of the dipyridamole-loaded PLG microspheres (as prepared in Example 95) and specific amounts of the prednisolone-loaded PLG microspheres (as prepared in Example 96) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added dipyridamole-loaded PLG microspheres and added prednisolone-loaded PLG microspheres is 200 mg. The ratio of dipyridamole-loaded PLG microspheres to prednisolone-loaded PLG microspheres is adjusted to give a range of dipyridamole and prednisolone ratios. Specific ratios that are prepared include 75:25 (wt/wt), 60:40, 50:50, 40:60, 25:75 dipyridamole:prednisolone.

### Example 98

### Dexamethasone-Loaded PLG Microspheres (<10 Micron)

800 mg PLG (85:15, Absorbable Polymers International) is dissolved in 20 ml dichloromethane. 125 mg dexamethasone is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg dexamethasone.

### Example 99

### Econazole-Loaded PLG Microspheres (<10 Micron)

800 mg PLG (85:15, Absorbable Polymers International) is dissolved in 20 ml dichloromethane. 125 mg econazole is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg econazole.

### Example 100

### Mixtures of Dexamethasone-Loaded PLG Microspheres and Econazole-Loaded PLG Microspheres (<10 Micron)

Mixtures of the dexamethasone-loaded PLG microspheres and econazole-loaded PLG microspheres are prepared by weighing out specific amounts of the dexamethasone-loaded PLG microspheres (as prepared in Example 98) and specific amounts of the econazole-loaded PLG microspheres (as prepared in Example 99) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added dexamethasone-loaded PLG microspheres and added econazole-loaded PLG microspheres is 200 mg. The ratio of dexamethasone-loaded PLG microspheres to econazole-loaded PLG microspheres is adjusted to give a range of dexamethasone and econazole ratios. Specific ratios that are prepared include 75:25 (wt/wt), 60:40, 50:50, 40:60, 25:75 dexamethasone:econazole.

### Example 101

### Diflorasone-Loaded PLG Microspheres (<10 Micron)

800 mg PLG (85:15, Absorbable Polymers International) is dissolved in 20 ml dichloromethane. 125 mg diflorasone is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg diflorasone.

### Example 102

### Alprostadil-Loaded PLG Microspheres (<10 Micron)

800 mg PLG (85:15, Absorbable Polymers International) is dissolved in 20 ml dichloromethane. 125 mg alprostadil is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg alprostadil.

### Example 103

### Mixtures of Diflorasone-Loaded PLG Microspheres and Alprostadil-Loaded PLG Microspheres (<10 Micron)

Mixtures of the diflorasone-loaded PLG microspheres and alprostadil-loaded PLG microspheres are prepared by weighing out specific amounts of the diflorasone-loaded PLG microspheres (as prepared in Example 101) and specific amounts of the alprostadil-loaded PLG microspheres (as prepared in Example 102) into a 6 ml glass scintillation vial (Sigma, Cat # Ml 152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added diflorasone-loaded PLG microspheres and added alprostadil-loaded PLG micro spheres is 200 mg. The ratio of diflorasone-loaded PLG micro spheres to alprostadil-loaded PLG microspheres is adjusted to give a range of diflorasone and alprostadil ratios. Specific ratios that are prepared include 75:25 (wt/wt), 60:40, 50:50, 40:60, 25:75 diflorasone:alprostadil.

### Example 104

### Dipyridamole-Loaded PLG Microspheres (<10 Micron)

800 mg PLG (85:15, Absorbable Polymers International) is dissolved in 20 ml dichloromethane. 125 mg dipyridamole is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

The process of preparing the microspheres is repeated using 160 mg, 100 mg, 75 mg and 40 mg dipyridamole.

### Example 105

### Amoxapine-Loaded PLG Microspheres (<10 Micron)

800 mg PLG (85:15, Absorbable Polymers International) is dissolved in 20 ml dichloromethane. 125 mg amoxapine is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg amoxapine.

### Example 106

### Mixtures of Dipyridamole-Loaded PLG Microspheres and Amoxapine-Loaded PLG Microspheres (<10 Micron)

Mixtures of the dipyridamole-loaded PLG microspheres and amoxapine-loaded PLG microspheres are prepared by weighing out specific amounts of the dipyridamole-loaded PLG microspheres (as prepared in Example 104) and specific amounts of the amoxapine-loaded PLG microspheres (as prepared in Example 105) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added dipyridamole-loaded PLG microspheres and added amoxapine-loaded PLG micro spheres is 200 mg. The ratio of dipyridamole-loaded PLG microspheres to amoxapine-loaded PLG microspheres is adjusted to give a range of dipyridamole and amoxapine ratios. Specific ratios that are prepared include 75:25 (wt/wt), 60:40, 50:50, 40:60, 25:75 dipyridamole:amoxapine.

### Example 107

### Dipyridamole-Loaded PLG Microspheres (<10 Micron)

800 mg PLG (85:15, Absorbable Polymers International) is dissolved in 20 ml dichloromethane. 125 mg dipyridamole is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg dipyridamole.

### Example 108

### Ibudilast-Loaded PLG Microspheres (<10 Micron)

800 mg PLG (85:15, Absorbable Polymers International) is dissolved in 20 ml dichloromethane. 125 mg ibudilast is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg ibudilast.

### Example 109

### Mixtures of Dipyridamole-Loaded PLG Microspheres and Ibudilast-Loaded PLG Microspheres (<10 Micron)

Mixtures of the dipyridamole-loaded PLG microspheres and ibudilast-loaded PLG microspheres are prepared by weighing out specific amounts of the dipyridamole-loaded PLG microspheres (as prepared in Example 107) and specific amounts of the ibudilast-loaded PLG microspheres (as prepared in Example 108) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added dipyridamole-loaded PLG microspheres and added ibudilast-loaded PLG microspheres is 200 mg. The ratio of dipyridamole-loaded PLG microspheres to ibudilast-loaded PLG microspheres is adjusted to give a range of dipyridamole and ibudilast ratios. Specific ratios that are prepared include 75:25 (wt/wt), 60:40, 50:50, 40:60, 25:75 dipyridamole:ibudilast.

### Example 110

### Nortriptyline-Loaded PLG Microspheres (<10 Micron)

800 mg PLG (85:15, Absorbable Polymers International) is dissolved in 20 ml dichloromethane. 125 mg nortriptyline is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg nortriptyline.

### Example 111

### Loratadine (or Desloratadine)-Loaded PLG Microspheres (<10 Micron)

800 mg PLG (85:15, Absorbable Polymers International) is dissolved in 20 ml dichloromethane. 125 mg loratadine (or desloratadine) is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours.

The process of preparing the microspheres is repeated using 160 mg, 100 mg, 75 mg and 40 mg loratadine (or desloratadine).

### Example 112

### Mixtures of Nortriptyline-Loaded PLG Microspheres and Loratadine (or Desloratadine)-Loaded PLG Microspheres (<10 Micron)

Mixtures of the nortriptyline-loaded PLG microspheres and loratadine (or desloratadine)-loaded PLG microspheres are prepared by weighing out specific amounts of the nortriptyline-loaded PLG microspheres (as prepared in Example 110) and specific amounts of the loratadine (or desloratadine)-loaded PLG Microspheres (as prepared in Example 111) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added nortriptyline-loaded PLG microspheres and added loratadine (or desloratadine)-loaded PLG microspheres is 200 mg. The ratio of nortriptyline-loaded PLG microspheres to loratadine (or desloratadine)-loaded PLG microspheres is adjusted to give a range of nortriptyline and loratadine (or desloratadine) ratios. Specific ratios that are prepared include 75:25 (wt/wt), 60:40, 50:50, 40:60, 25:75 nortriptyline:loratadine (or desloratadine).

### Example 113

### Albendazole-Loaded PLG Microspheres (<10 Micron)

800 mg PLG (85:15, Absorbable Polymers International) is dissolved in 20 ml dichloromethane. 125 mg albendazole is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg albendazole.

### Example 114

### Pentamidine-Loaded PLG Microspheres (<10 Micron)

800 mg PLG (85:15, Absorbable Polymers International) is dissolved in 20 ml dichloromethane. 125 mg pentamidine is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg pentamidine.

### Example 115

### Mixtures of Albendazole-Loaded PLG Microspheres and Pentamidine-Loaded PLG Microspheres (<10 Micron)

Mixtures of the albendazole-loaded PLG microspheres and pentamidine-loaded PLG microspheres are prepared by weighing out specific amounts of the albendazole-loaded PLG microspheres (as prepared in Example 113) and specific amounts of the pentamidine-loaded PLG Microspheres (as prepared in Example 114) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added albendazole-loaded PLG microspheres and added pentamidine-loaded PLG micro spheres is 200 mg. The ratio of albendazole-loaded PLG microspheres to pentamidine-loaded PLG microspheres is adjusted to give a range of albendazole and pentamidine ratios. Specific ratios that are prepared include 75:25 (wt/wt), 60:40, 50:50, 40:60, 25:75 albendazole:pentamidine.

### Example 116

### Itraconazole-Loaded PLG Microspheres (<10 Micron)

800 mg PLG (85:15, Absorbable Polymers International) is dissolved in 20 ml dichloromethane. 125 mg itraconazole is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg itraconazole.

### Example 117

### Lovastatin-Loaded PLG Microspheres (<10 Micron)

800 mg PLG (85:15, Absorbable Polymers International) is dissolved in 20 ml dichloromethane. 125 mg lovastatin is added to the dissolved polymer solution. 100 ml of freshly prepared 10% polyvinyl alcohol (PVA) solution is added into a 600 ml beaker. The PVA solution is stirred at 2000 rpm for 30 minutes. The polymer/dichloromethane solution is added dropwise to the PVA solution while stirring at 2000 rpm with a Fisher DYNA-MIX stirrer. After addition is complete, the solution is allowed to stir for an additional 3 hours. The microsphere solution is transferred to several disposable 50 ml graduated polypropylene conical centrifuge tubes and is centrifuged at 2600 rpm for 10 minutes. The aqueous layer is decanted and the microspheres are resuspended with deionized water. The centrifugation, decanting and resuspending steps are repeated 3 times. The combined, washed microspheres are transferred to a single centrifuge tube, frozen in an acetone/dry-ice bath and then freeze-dried. Following the freeze drying process, the microspheres are further dried under vacuum for about 24 hours. The process of preparing the microspheres is repeated using 160 mg, 100 mg, 75 mg and 40 mg lovastatin.

### Example 118

### Mixtures of Itraconazole-Loaded PLG Microspheres and Lovastatin-Loaded PLG Microspheres (<10 Micron)

Mixtures of the itraconazole-loaded PLG microspheres and lovastatin-loaded PLG microspheres are prepared by weighing out specific amounts of the itraconazole-loaded PLG microspheres (as prepared in Example 116) and specific amounts of the lovastatin-loaded PLG microspheres (as prepared in Example 117) into a 6 ml glass scintillation vial (Sigma, Cat # M1152). The cap is placed on the vial and the microspheres are vortexed for 1 min. The vial is then inverted and is tapped a few times to ensure than most of the microspheres fell to the lid end of the vial. The vial is inverted. The vortex/inversion process is repeated 4 times. The specific masses used are chosen such that a sum of the added itraconazole-loaded PLG microspheres and added lovastatin-loaded PLG micro spheres is 200 mg. The ratio of itraconazole-loaded PLG microspheres to lovastatin-loaded PLG microspheres is adjusted to give a range of itraconazole and lovastatin ratios. Specific ratios that are prepared include 75:25 (wt/wt), 60:40, 50:50, 40:60, 25:75 itraconazole:lovastatin.

### Example 119

### Effects of the Combination of Methyl Prednisolone Acetate and Amoxapine in a Rat Carrageenan-Induced Paw Edema Model

A dose-range finding study was performed to determine the anti-inflammatory activity of various ratios of methyl prednisolone acetate and amoxapine in a rat carrageenan-induced paw edema model. The end points of assessment included inhibition of paw swelling at the time of maximum swelling (Tₘₐₓ=6 hours) and down regulation of the pro-inflammatory cytokine TNF-α in the paw tissue. The molar ratio of methyl prednisolone acetate to amoxapine ranged from 1:1 to 1:300, using total doses of methyl prednisolone acetate of 0.01, 0.03 or 0.1 mg/kg.

The test agent (a combination of methyl prednisolone acetate and amoxapine), vehicle control, or reference agents (methyl prednisolone acetate, amoxapine, or Depo-Medrol®) were administered in the left hind foot pad of rats. After 60 minutes, paw edema was induced by injection of 100 μl of 1% carrageenan in the same foot pad. The paw volume was measured with a water displacement plethysmometer immediately prior to test agent injection (T₋₁ₕ), at the time of carrageenan injection (T₀ₕ), and at T₆ₕ. Animals were euthanized by carbon dioxide inhalation. Paw tissue samples were collected and flash frozen in liquid nitrogen. Samples were assayed for TNF-α by enzyme-linked immunoassay (ELISA). The data are shown in the Table 14 below.

TABLE 14
Results of Carrageenan-Induced Paw Edema Study

| | Edema^{b} ± SEM | | TNF-α^{d} ± SEM | |
|---|---|---|---|---|
| Groups^{a} | (%) | p-value^{c} | (pg/g) | p-value^{e} |
| Vehicle (diluent, negative | 49.6 ± 4.4 | — | 59.9 ± 13.1 | — |
| control) | | | | |
| Depo-Medrol (positive control) 1 mg/kg | 15.3 ± 3.0 | <0.001 | 21.9 ± 6.3 | 0.01 |
| Amoxapine 2.26 mg/kg | 38.1 ± 3.3 | NS | 32.6 ± 10.1 | 0.05 |
| MePredAc 0.01 mg/kg | 32.6 ± 5.3 | 0.03 | 35.9 ± 11.3 | 0.001 |
| MePredAc 0.03 mg/kg | 26.2 ± 7.0 | 0.02 | 19.2 ± 3.1 | 0.01 |
| MePredAc 0.1 mg/kg | 12.2 ± 1.8 | <0.001 | 28.0 ± 6.0 | 0.06 |
| MePredAc 0.01 mg/kg + Amox | 48.4 ± 3.8 | NS | 47.5 ± 8.8 | NS |
| 2.26 mg/kg | | | | |
| MePredAc 0.03 mg/kg + Amox | 24.3 ± 4.5 | 0.001 | 27.8 ± 3.7 | 0.04 |
| 0.753 mg/kg | | | | |
| MePredAc 0.03 mg/kg + Amox | 13.6 ± 1.7 | <0.001 | 14.6 ± 4.1 | 0.01 |
| 2.26 mg/kg | | | | |
| MePredAc 0.1 mg/kg + Amox | 22.2 ± 6.6 | 0.01 | 22.5 ± 5.7 | 0.01 |
| 0.753 mg/kg | | | | |
| MePredAc 0.1 mg/kg + Amox | 12.5 ± 2.2 | <0.001 | 9.4 ± 2.6 | 0.01 |
| 2.26 mg/kg | | | | |

^{a}All animals pre-treated with drugs at T-1 hr, at T0 hrs animals were injected with 1% Carrageenan (100 μl) by local injection into the paws. Vehicle Group n = 11 rats/group, other groups at n = 8 rats/group.
^{b}% Edema following carrageenan induction at Tmax = 6 hrs, SEM = standard error of the mean
^{c}p-value for edema vs. vehicle control, NS = not significant
^{d}TNF-α measured by ELISA in the paw tissues of carrageenan injected paws
^{e}p-value for TNF-α vs. vehicle control, NS = not significant

Carrageenan-injected paws treated with the vehicle (control) exhibited a ˜50% increase in paw volume. Administration of the clinical agent Depo-Medrol (1 mg/kg) significantly inhibited paw edema (p<0.001) reducing it to the background level of ˜15%. Treatment with amoxapine (Amox) alone at 2.26 mg/kg was not significantly different from the vehicle treatment. Groups treated with methyl prednisolone acetate (MePredAc) alone showed a dose-dependent reduction in paw edema following treatment.

A combination containing 0.03 mg/kg MePredAc with an amoxapine dose of 2.26 mg/kg significantly enhanced MePredAc effects, bringing down the edema levels to 13.6%±1.7. This is equivalent to the effect observed using Depo-Medrol®, but with a much lower steroid dose.

### Example 120

### Effect of Amoxapine and Prednisolone in the Rat SCW-Induced Model of Polyarthritis

Cromatie et al. (Cromatie W J, Craddock J G, Schwab J H, Anderlie S K, and Yang C. Arthritis in rats after systemic injection of streptococcal cells or cell walls. J Exp Med 146, 1585-1602, 1977) described streptococcal cell wall (“SCW”) arthritis in the 1970s when they noted that a single intraperitoneal injection of the SCW component peptidoglycan-polysaccharide (PG-PS), suspended in an aqueous phase will induce a chronic, severe, erosive arthritis in female Lewis rats. Typically, this model exhibits a peripheral and symmetrical, biphasic polyarthritis with cycles of exacerbated recurrence and remission and is clinically and histologically similar to rheumatoid arthritis. The incidence of acute and chronic arthritis is approximately 95-100% with a proper preparation of SCW 10S PG-PS fragments (15 μg rhamnose/g body wt) and healthy female Lewis rats. During the first 24 h, a T cell independent, possibly complement-dependent acute phase develops followed by visible joint swelling by day 3. The joint inflammation progresses from an initial acute phase (1-5 days) through a remission phase (typically around day 10), followed by a spontaneous reactivation phase. The reactivation phase typically starts around day 14 and is characterized by a significant increase in both arthritis index and paw volume. By day 28, joint inflammation in both hindlimbs and forelimbs is well established and becomes chronic by 4 weeks and persists for months (Kannan K, Ortmann R A, Kimpel D. Animal models of rheumatoid arthritis and their relevance to human disease. Pathophysiology 12, 167-181, 2005). Some pathological changes observed in SCW-induced arthritis that are of relevance to human RA include infiltration of polymorphonuclear cells, CD4+ T cells and macrophages, hyperplasia of the synovial lining layer, pannus formation and moderate erosion of cartilage and bone. Previous reports have shown the dependency of this model on tumour necrosis factor (TNF)-α, IL-4, P-selectin, IL-1β, IL-6, macrophage inflammatory protein (MIP)-2, MIP-1α and monocyte chemoattractant protein (MCP)-1 and other key pro-inflammatory cytokines and chemokines (Rioja I, Clayton C L, Graham S J, Life P F and Dickson M C. Gene expression profiles in the rat streptococcal cell wall-induced arthritis model identified using microarray analysis. Arthritis Res Ther 7:R101-R117, 2005).

Using this arthritis model the efficacy of the combination of methyl prednisolone acetate and amoxapine on the development of arthritis in the SCW model of arthritis in female Lewis rats is demonstrated. The combination is injected intra-articularly into the ankle joint at the onset of the spontaneous reactivation phase, typically around Day 10 to 14 post arthritis induction, and animals are monitored for inflammatory score, paw volume, ankle joint swelling and body weight until sacrifice, typically 7 days later. At sacrifice measurements of serum TNF-α levels and histology inflammation scores are obtained.

Induction of Arthritis

Rats in the assay and positive control groups are each injected with typically 15 μg of rhamnose/gm average body weight. The injection is prepared aseptically to avoid contamination. Prior to injection anaesthesia is induced by exposure to 5% isoflurane and maintained at 1-2% isoflurane by use of a nose cone. Each rat is injected intraperitoneally in the lower left of the abdomen, taking care to in the placement of the needle to avoid injecting the PG-PS into either the stomach or the caecum. A 1 mL syringe with a 23 gauge needle is used. The negative control rats are injected in the same manner, using the same volume of sterile 0.9% saline instead of the PG-PS. The rats are observed on a daily basis, and monitored three times a week. Monitoring consists of in life observations, body weights, plethysmographic measurement of paw volume, determination of peri-articular swelling, and visual scoring, for example using the arthritic index (Al) score. The Al is based on the degree of swelling, erythema, and deformity of the joint and is similar to the scoring system used in the clinic. Each limb is assessed on a scale of 0-4 and the scores are added for all four limbs resulting in a maximum score of 16 for each animal. If the severity of the arthritis indicates the need for greater pain management, a subcutaneous injection of buprenorphine (0.01 mg) is given whenever required during the study.

Test Article Administration

Control groups are defined as those rats receiving saline (negative control) and those rats receiving PG-PS (positive control) with no intervention or treatment. Assay groups are defined as rats receiving PG-PS with intervention. The test article, a combination of amoxapine (Medichem S. A., Girona, Spain) and methylprednisolone acetate (Aventis Pharma, Vertolaye, France), is administered by direct intra-articular injection in the ankle at the onset of the spontaneous reactivation phase, typically around Day 10 to 14 post arthritis induction. The rats are randomized into treatment groups, depending on ankle diameter, so that the average ankle diameter in each group is approximately the same. Before each use, the vial for each test article is inverted gently and repeatedly for approximately 10 seconds to achieve uniform suspension. A 25-27 gauge needle is used to withdraw the suspension from the vial for administration. A new 25-27 gauge needle is used for the injection. After induction and maintenance of anaesthesia, a 27 gauge 0.5 inch needle is inserted though the Achilles tendon above the calcaneous into the vicinity of the tibiotalar joint. The test article or vehicle is injected in a volume of approximately 0.05 mL into the most swollen ankle of each rat. Typical doses are 0.01 to 0.3 mg/kg methylprednisolone acetate in combination with 0.2 to 2.0 mg/kg amoxapine, evaluated in comparison with doses of 0.01 to 3.0 mg/kg methylprednisolone acetate alone and 0.2 to 2.0 mg/kg amoxapine alone. After intra-articular injection, the animals continue to be monitored, as described above, three times a week until sacrifice.

Data Collection Methods

Arthritis Monitoring

Rats are monitored for severity of arthritis three times a week following arthritis induction. Monitoring consists of measurement of paw swelling using a plethysmograph (Model # 7140, UGO BASILE Biological Research Apparatus) and measurement of ankle diameter using digital calipers (Pro-max cal electronic caliper, Fred Fowler Co, Inc). In addition a visual assessment is made of all four limbs.

TNF-α Levels

Animals injected on Day 0 with 0.9% saline IP instead of PG-PS are sacrificed on the day of treatment (around Day 10 to 14) to obtain normal histological specimens and baseline TNF-α levels. For all other groups, all animals are sacrificed seven days following the day of treatment. Blood is obtained by cardiac puncture at time of sacrifice, spun down and the plasma removed. TNF-α levels in plasma are determined by ELISA (R&D, Minneapolis Minn.).

Results obtained for the efficacy of test article in this model are analysed using standard statistical methods.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A composition for local subcutaneous administration to a patient for treatment of edema, comprising a corticosteroid in an amount effective to inhibit edema, and a tricyclic antidepressant, wherein the tricyclic antidepressant is present in an amount effective to enhance the inhibitory effect of the corticosteroid.

2. The composition of claim 1, wherein the corticosteroid comprises methylprednisolone acetate in the amount of at least 0.03 mg/kg, and the tricyclic antidepressant comprises amoxapine in the amount of up to about 2.26 mg/kg.

3. A composition for local subcutaneous administration to a patient for treatment of inflammation, comprising a corticosteroid and a tricyclic antidepressant, wherein the tricyclic antidepressant is present in an amount effective to promote an anti-inflammatory effect of the corticosteroid.

4. The composition of claim 3, wherein the corticosteroid comprises methylprednisolone acetate in the amount of at least 0.03 mg/kg, and the tricyclic antidepressant comprises amoxapine in the amount of up to about 2.26 mg/kg.

5. A composition for local subcutaneous administration to a patient for treatment of local inflammation, comprising a corticosteroid in an amount effective to inhibit inflammation and a tricyclic antidepressant, wherein the tricyclic antidepressant is present in an amount effective to enhance the inhibitory effect of the corticosteroid.

6. The composition of claim 5, wherein the corticosteroid comprises methylprednisolone acetate in the amount of up to about 0.3 mg/kg, and the tricyclic antidepressant comprises amoxapine in the amount of up to about 2.26 mg/kg.

7. A composition for intraarticular administration to a patient for treatment of inflammation, comprising a corticosteroid in an amount effective to inhibit edema, and a tricyclic antidepressant, wherein the tricyclic antidepressant is present in an amount effective to enhance the inhibitory effect of the corticosteroid.

8. The composition of claim 7, wherein the corticosteroid comprises methylprednisolone acetate in the amount of up to about 0.3 mg/kg, and the tricyclic antidepressant comprises amoxapine in the amount of up to about 2.0 mg/kg.

9. A composition for intraarticular administration to a patient for treatment of inflammation, comprising a corticosteroid and a tricyclic antidepressant, wherein the tricyclic antidepressant is present in an amount effective to promote an anti-inflammatory effect of the corticosteroid.

10. The composition of claim 9, wherein the corticosteroid comprises methylprednisolone acetate in the amount of up to about 0.3 mg/kg, and the tricyclic antidepressant comprises amoxapine in the amount of up to about 2.0 mg/kg.

11. The composition of claim 10, wherein the amount of methylprednisolone acetate is in the range of 0.01 mg/kg to 0.3 mg/kg and the amount of amoxapine is in the range of 0.2 to 2.0 mg/kg.

12. A composition for intraarticular administration to a patient for treatment of inflammation, comprising a corticosteroid in an amount effective to inhibit inflammation and a tricyclic antidepressant, wherein the tricyclic antidepressant is present in an amount effective to enhance the inhibitory effect of the corticosteroid.

13. The composition of claim 12, wherein the corticosteroid comprises methylprednisolone acetate in the amount of up to about 0.03 mg/kg, and the tricyclic antidepressant comprises amoxapine in the amount of up to about 2.0 mg/kg.

14. The composition of claim 13, wherein the amount of methylprednisolone acetate is in the range of 0.01 mg/kg to 0.3 mg/kg and the amount of amoxapine is in the range of 0.2 to 2.0 mg/kg.
